(19) United States
(12) Patent Application Publication
(10) Pub. No.: US 2026/0356841 A1
(43) Pub. Date: Nov. 12, 2026

(54) HETEROCYCLIC PYRIDINONE COMPOUNDS AS TRIGGERING RECEPTOR EXPRESSED ON MYELOID CELLS 2 AGONISTS AND METHODS OF USE

(71) Applicant: Vigil Neuroscience, Inc., Morristown, NJ (US)

(72) Inventors: Jonathan B. Houze, Morristown, NJ (US); Bhaumik Pandya, Watertown, NJ (US)

(21) Appl. No.: 19/706,505

(22) Filed: Jun. 12, 2026

Related U.S. Application Data
(63) Continuation of application No. PCT/US24/59856, filed on Dec. 12, 2024.
(60) Provisional application No. 63/609,320, filed on Dec. 12, 2023.
(60) Provisional application No. 63/609,327, filed on Dec. 12, 2023.

(51) Int. Cl. C07D 487/04; A61K 31/444; A61K 31/4985; A61K 31/52; A61K 31/5377; C07B 59/00; C07D 471/04; C07D 473/00; C07D 513/04

(52) U.S. Cl. CPC C07D 487/04; A61K 31/444; A61K 31/4985; A61K 31/52; A61K 31/5377; C07B 59/002; C07D 471/04; C07D 473/00; C07D 513/04; C07B 2200/05

(57) ABSTRACT

The present disclosure provides compounds of Formula (I), useful for the activation of Triggering Receptor Expressed on Myeloid Cells 2 (“TREM2”).

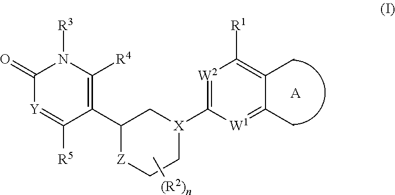

This disclosure also provides pharmaceutical compositions comprising the compounds, uses of the compounds, and compositions for treatment of, for example, a neurodegenerative disorder. Further, the disclosure provides intermediates useful in the synthesis of compounds of Formula (I).

### CROSS REFERENCE TO RELATED APPLICATION

This application is a continuation of International Patent Application No. PCT/US2024/059856 filed on Dec. 12, 2024, which claims the benefit under 35 U.S.C. § 119 (e) to U.S. Provisional Application No. 63/609,327, filed Dec. 12, 2023, and to U.S. Provisional Application No. 63/609,320, filed Dec. 12, 2023, the entirety of each of which is incorporated herein by reference.

### FIELD

The present disclosure provides compounds useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula (I).

### BACKGROUND

Microglia are resident innate immune cells in the brain and are important for the maintenance of homeostatic conditions in the central nervous system (Hickman et al. Nat Neurosci 2018, Li and Barres, Nat Rev Immunol., 2018). These resident macrophages express a variety of receptors that allow them to sense changes in their microenvironment and alter their phenotypes to mediate responses to invading pathogens, proteotoxic stress, cellular injury, and other infarcts that can occur in health and disease. Id. Microglia reside in the parenchyma of the brain and spinal cord where they interact with neuronal cell bodies (Cserep et al. Science, 2019), neuronal processes (Paolicelli et al. Science, 2011, Ikegami et al. Neruopathology, 2019) in addition to other types of glial cells (Domingues et al. Front Cell Dev Biol, 2016; Liddelow et al. Nature, 2017, Shinozaki et al. Cell Rep., 2017), playing roles in a multitude of physiological processes. With the ability to rapidly proliferate in response to stimuli, microglia characteristically exhibit myeloid cell functions such as phagocytosis, cytokine/chemokine release, antigen presentation, and migration (Colonna and Butovsky, Annu Rev Immunol, 2017). More specialized functions of microglia include the ability to prune synapses from neurons and directly communicate with their highly arborized cellular processes that survey the area surrounding the neuronal cell bodies (Hong et al. Curr Opin Neurobiol, 2016; Sellgren et al. Nat Neurosci, 2019).

The plasticity of microglia and their diverse states as described through single-cells RNASeq profiling are thought to arise through the integration of signaling from a diverse array of cell surface receptors (Hickman et al. Nat Neurosci 2013). Collectively known as the microglial “sensome,” these receptors are responsible for transducing activating or activation-suppressing intracellular signaling and include protein families such as Sialic acid-binding immunoglobulin-type lectins (“SIGLEC”), Toll-like receptors (“TLR”), Fc receptors, nucleotide-binding oligomerization domain (“NOD”) and purinergic G protein-coupled receptors. Doens and Fernandez 2014, Madry and Attwell 2015, Hickman and E1 Khoury 2019. Similar to other cells of the myeloid lineage, the composition of microglial sensomes is dynamically regulated and acts to recognize molecular pattern that direct phenotypic responses to homeostatic changes in the central nervous system (“CNS”). Id. One of the receptors selectively expressed by brain microglia is TREM2, composed of a single-pass transmembrane domain, an extracellular stalk region, and extracellular immunoglobulin variable (“IgV”)-like domain responsible for ligand interaction (Kleinberger et al. Sci Transl Med, 2014). As TREM2 does not possess intracellular signal transduction-mediating domains, biochemical analysis has illustrated that interaction with adaptor proteins DAP10 and DAP12 mediate downstream signal transduction following ligand recognition (Peng et al. Sci Signal 2010; Jay et al. Mol Neurodegener, 2017). TREM2/DAP12 complexes in particular act as a signaling unit that can be characterized as pro-activation on microglial phenotypes in addition to peripheral macrophages and osteoclasts (Otero et al. J Immunol, 2012; Kobayashi et al. J Neurosci, 2016; Jaitin et al., Cell, 2019. In the CNS, signaling through TREM2 has been studied in the context of ligands such as phospholipids, cellular debris, apolipoproteins, and myelin (Wang et al. Cell, 2015; Kober and Brett, J Mol Biol, 2017; Shirotani et al., Sci Rep, 2019). In mice lacking functional TREM2 expression or expressing a mutated form of the receptor, a core observation is blunted microglial responses to insults such as oligodendrocyte demyelination, stroke-induced tissue damage in the brain, and proteotoxic inclusions in vivo (Cantoni et al., Acta Neuropathol, 2015, Wu et al., Mol Brain, 2017).

Coding variants in the TREM2 locus has been associated with late onset Alzheimer's disease (“LOAD”) in human genome-wide association studies, linking a loss-of-receptor function to a gain in disease risk (Jonsson et al. N Engl J Med 2013, Sims et al. Nat Genet 2017). Genetic variation of other genes selectively expressed by microglia in the CNS, for example, CD33, PLCg2 and MS4A4A/6A have reached genome-wide significance for their association with LOAD risk (Hollingworth et al. Nat Genet 2011, Sims et al. Nat Genet 2017, Deming et al. Sci Transl Med 2019). Together, these genetic findings link together in a putative biochemical circuit that highlights the importance of microglial innate immune function in LOAD. Additionally, increase or elevation in the soluble form of TREM2 (“sTREM2”) in the cerebrospinal fluid (CSF) of human subjects is associated with disease progression and emergence of pathological hallmarks of LOAD including phosphorylated Tau (Suarez-Calvet et al. Mol Neurodegener 2019). Furthermore, natural history and human biology studies indicate that baseline sTREM2 levels in the CSF can stratify the rate of temporal lobe volume loss and episodic memory decline in longitudinally monitored cohorts (Ewers et al. Sci Transl Med 2019).

In addition to human genetic evidence supporting a role of TREM2 in LOAD, homozygous loss-of-function mutations in TREM2 are causal for an early onset dementia syndrome known as Polycystic lipomembranous osteodysplasia with sclerosing leukoencephalopathy (“PLOSL”) or Nasu-Hakola disease (“NHD”)(Golde et al. Alzheimers Res Ther 2013, Dardiotis et al. Neurobiol Aging 2017). This progressive neurodegenerative disease typically manifests in the 3^{rd} decade of life and is pathologically characterized by loss of myelin in the brain concomitant with gliosis, unresolved neuroinflammation, and cerebral atrophy. Typical neuropsychiatric presentations are often preceded by osseous abnormalities, such as bone cysts and loss of peripheral bone density (Bianchin et al. Cell Mol Neurobiol 2004; Madry et al. Clin Orthop Relat Res 2007, Bianchin et al. Nat Rev Neurol 2010). Given that osteoclasts of the myeloid lineage are also known to express TREM2, the PLOSL-related symptoms of wrist and ankle pain, swelling, and fractures indicate that TREM2 may act to regulate bone homeostasis through defined signaling pathways that parallel the microglia in the CNS (Paloneva et al. J Exp Med 2003, Otero et al. J Immunol 2012). The link between TREM2 function and PLOSL has illustrated the importance of the receptor in sustaining key physiological aspects of myeloid cell function in the human body.

Efforts have been made to model the biology of TREM2 in mice prompting the creation of TREM2 knock out (“KO”) mice in addition to the LOAD-relevant TREM2 R47H loss-of-function mutant transgenic mice (Ulland et al. Cell, 2017, Kang et al. Hum Mol Genet 2018). Although unable to recapitulate the neurological manifestations of PLOSL, TREM2 KO mice show abnormalities in bone ultrastructure (Otero et al. J Immunol 2012). When the TREM2 KO or mutant mice have been crossed onto familial Alzheimer's disease transgenic mouse background such as the 5×FAD amyloidogenic mutation lines, marked phenotypes have been observed (Ulrich et al. Neuron, 2017). These in vivo phenotypes of TREM2 loss-of-function in the CNS include elevated the plaque burden and lower levels of secreted microglial factors SPP1 and Osteopontin that are characteristic of the microglial response to amyloid pathology (Ulland et al. Cell, 2017). Other rodent studies have demonstrated that loss of TREM2 leads to decreased microglial clustering around plaques and emergence of less compact plaque morphology in familial AD amyloid models (Parhizkar et al. Nat Neurosci 2019). With regards to the Tau protein pathology that is observed in LOAD, familial tauopathy models in mice demonstrated an enhanced spreading of pathological human Tau aggregates from point of injection into mouse brain in TREM2 KO mice (Leyns et al. Nat Neurosci 2019). Furthermore, single-cell RNASeq studies with the TREM2 KO mice in aged scenarios, 5×FAD familial Alzheimer's disease model mice, and Amyotrophic Lateral Sclerosis SODI mutant mouse backgrounds indicate that TREM2 receptor function is critical for a conserved set of phenotypic transformations within microglial populations in response to CNS pathology (Keren-Shaul et al. Cell 2017).

In rodent models where TREM2 expression levels are elevated, brain amyloid pathology in the 5×FAD transgenic mice displayed reduced plaque volume and altered morphology (Lee et al. Neuron, 2018). The changes in immunohistological markers relating to brain amyloid pathology were also accompanied by an attenuated presence of dystrophic neurites when TREM2 was overexpressed. Id. Therefore, the pharmacological activation of TREM2 is a target of interest for treating or preventing neurological, neurodegenerative and other diseases. Despite many attempts to alter disease progression by targeting the pathological hallmarks of LOAD through anti-amyloid and anti-Tau therapeutics, there is a need for activators of TREM2 to address the genetics-implicated neuroimmune aspects of, for example, LOAD. Such TREM2 activators may be suitable for use as therapeutic agents and remain in view of the significant continuing societal burden that remains unmitigated for diseases.

### SUMMARY

First, provided herein is a compound of Formula (I)

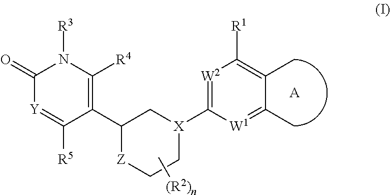

or a pharmaceutically acceptable salt, solvate, stereoisomer, or tautomer thereof, or a pharmaceutically acceptable salt and/or solvate of said compound, stereoisomer, or tautomer, wherein:
Ring A together with the 6-membered ring system to which it is fused forms a bicyclic ring system selected from one of:

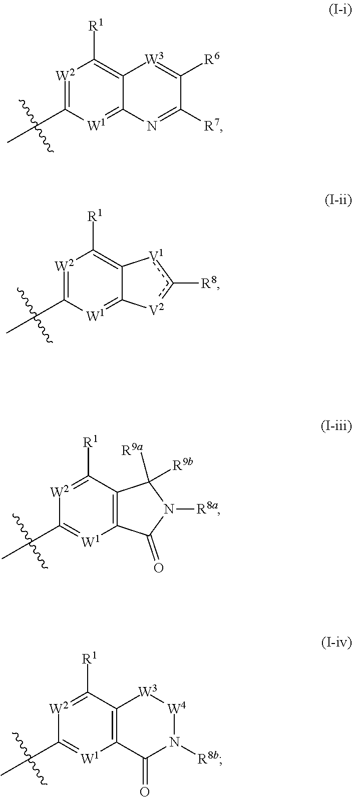

V¹ and V² are each independently N, NR^{8b}, or S, wherein both V¹ and V² are not S, and wherein the bonds in the ring comprising V¹ and V² are either single or double bonds, depending on the valency of V¹ and V²;
W¹, W², W³, and W⁴ are each independently C(R¹⁰), N, or NR⁸¹, wherein the bonds connecting W³ to W⁴ and W⁴ to NR^{8b} are either single or double bonds, depending on the valency of W³ and W⁴;
X is C(R¹¹) or N;
Y is C(R¹²) or N;
Z is C(R¹⁸)₂ or O;

R¹ is cycloalkyl, heterocyclyl, aryl, or heteroaryl, each of which is optionally substituted with one or more R¹³;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R¹⁴; or
two R², taken together with the carbon atom to which they are attached, form an oxo group;
R³ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁵;
R⁴ and R⁵ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁴;
R⁶ and R⁷ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁶;
R⁸ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, or N(R^{B})(R^{C}), wherein alkyl, alkenyl, alkynyl, heteroalkyl, haloalkyl, cycloalkyl, and heterocyclyl is optionally substituted with one or more R¹⁶;
R^{8a} is hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶;
R^{8b} is absent, hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶;
R^{9a}, R^{9b}, and R^{9c} are independently in each instance hydrogen, deuterium, C₁₋₆ alkyl, C₂₋₆ alkenyl, C₂₋₆ alkynyl, C₁₋₆ heteroalkyl, C₁₋₆ haloalkyl or halogen;
R¹⁰, R¹¹, and R¹² are each independently in each instance hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, or heteroaryl, halogen, cyano, —O(R^{A}), or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁶,
each R¹³ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —C(O)N(R^{B})(R^{C}), or —N(R^{B})(R^{C}), wherein each alkyl, alkenyl, alkynyl, heteroalkyl, haloalkyl, cycloalkyl, and heterocyclyl is optionally substituted with one or more R¹⁷;
each R¹⁴, R¹⁵, and R¹⁶ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁷; or
two R¹⁴, two R¹⁵, or two R¹⁶, together with the carbon atom to which they are attached, form a cycloalkyl, heterocyclyl, aryl, or heteroaryl, wherein each cycloalkyl, heterocyclyl, aryl, and heteroaryl is optionally substituted with one or more R¹⁷;
each R^{A} is independently hydrogen, deuterium, C₁₋₆ alkyl, C₁₋₆ heteroalkyl, C₁₋₆ haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₂₋₆ alkenyl, C₁₋₆ heteroalkyl, C₁₋₆ haloalkyl, cycloalkyl, heterocyclyl, or —C(O)-alkyl, wherein each alkyl, alkenyl, heteroalkyl, haloalkyl, cycloalkyl, and heterocyclyl is optionally substituted with one or more R¹⁷; or
R^{B} and R^{C}, together with the nitrogen atom to which they are attached, form —N═C(R^{D})(R^{E}) or a heterocyclyl, wherein the heterocyclyl is optionally substituted with one or more R¹⁷;
each R^{D} and R^{E} is independently hydrogen, C₁₋₆ alkyl, C₂₋₆ alkenyl, C₁₋₆ heteroalkyl, C₁₋₆ haloalkyl, cycloalkyl, or heterocyclyl, wherein each alkyl, alkenyl, heteroalkyl, haloalkyl, cycloalkyl, and heterocyclyl is optionally substituted with one or more R¹⁷; or
each R¹⁷ is deuterium, C₁₋₆ alkyl, C₁₋₆ heteroalkyl, C₁₋₆ haloalkyl, cycloalkyl, halogen, or cyano; or
two R¹⁷ form an oxo;
each R¹⁸ is independently H, C₁₋₆ alkyl, or halo; and
n is 0, 1, 2, 3, 4, 5, or 6.

Second, provided herein is a pharmaceutical composition comprising a compound of Formula (I), a pharmaceutically acceptable salt, solvate, stereoisomer, or tautomer thereof, or a pharmaceutically acceptable salt and/or solvate of said compound, stereoisomer, or tautomer, and a pharmaceutically acceptable excipient.

Third, provided herein is a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition as described hereinabove, for use in treating or preventing a condition associated with a loss of function of human TREM2.

Fourth, provided herein is a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition described hereinabove, for use in treating or preventing Parkinson's disease, rheumatoid arthritis, Alzheimer's disease, Nasu-Hakola disease, frontotemporal dementia, multiple sclerosis, prion disease, or stroke.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein is a compound of Formula (I):

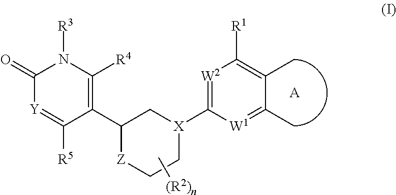

or a pharmaceutically acceptable salt, solvate, stereoisomer, or tautomer thereof, or a pharmaceutically acceptable salt and/or solvate of said compound, stereoisomer, or tautomer, wherein:
Ring A together with the 6-membered ring system to which it is fused forms a bicyclic ring system selected from one of:

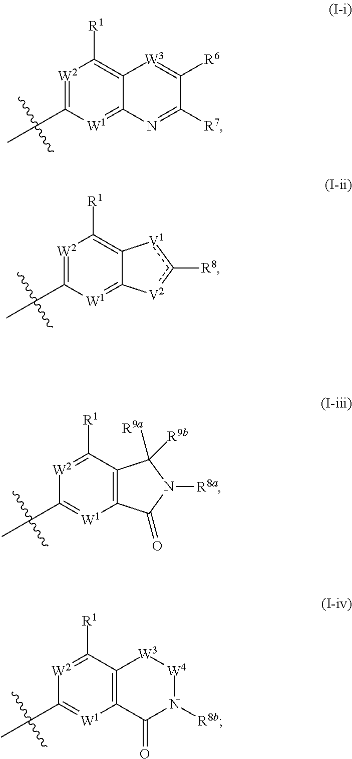

V¹ and V² are each independently N, NR^{8b}, or S, wherein both V¹ and V² are not S, and wherein the bonds in the ring comprising V¹ and V² are either single or double bonds, depending on the valency of V¹ and V²;
W¹, W², W³, and W⁴ are each independently C(R¹⁰), N, or NR^{8b}, wherein the bonds connecting W³ to W⁴ and W⁴ to NR^{8b} are either single or double bonds, depending on the valency of W³ and W⁴;
X is C(R¹¹) or N;
Y is C(R¹²) or N;
Z is C(R¹⁸)₂ or O;
R¹ is cycloalkyl, heterocyclyl, aryl, or heteroaryl, each of which is optionally substituted with one or more R¹³;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R¹⁴; or
two R², taken together with the carbon atom to which they are attached, form an oxo group;
R³ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁵;
R⁴ and R⁵ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁴;
R⁶ and R⁷ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁶;
R⁸ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, or N(R^{B})(R^{C}), wherein alkyl, alkenyl, alkynyl, heteroalkyl, haloalkyl, cycloalkyl, and heterocyclyl is optionally substituted with one or more R¹⁶;
R^{8a} is hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶;

R^{8b} is absent, hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶;
R^{9a}, R^{9b}, and R^{9c} are independently in each instance hydrogen, deuterium, C₁₋₆ alkyl, C₂₋₆ alkenyl, C₂₋₆ alkynyl, C₁₋₆ heteroalkyl, C₁₋₆ haloalkyl or halogen;
R¹⁰, R¹¹, and R¹² are each independently in each instance hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, or heteroaryl, halogen, cyano, —O(R^{A}), or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁶,
each R¹³ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —C(O)N(R^{B})(R^{C}), or —N(R^{B})(R^{C}), wherein each alkyl, alkenyl, alkynyl, heteroalkyl, haloalkyl, cycloalkyl, and heterocyclyl is optionally substituted with one or more R¹⁷;
each R¹⁴, R¹⁵, and R¹⁶ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁷; or
two R¹⁴, two R¹⁵, or two R¹⁶, together with the carbon atom to which they are attached, form a cycloalkyl, heterocyclyl, aryl, or heteroaryl, wherein each cycloalkyl, heterocyclyl, aryl, and heteroaryl is optionally substituted with one or more R¹⁷;
each R^{A} is independently hydrogen, deuterium, C₁₋₆ alkyl, C₁₋₆ heteroalkyl, C₁₋₆ haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₂₋₆ alkenyl, C₁₋₆ heteroalkyl, C₁₋₆ haloalkyl, cycloalkyl, heterocyclyl, or —C(O)-alkyl, wherein each alkyl, alkenyl, heteroalkyl, haloalkyl, cycloalkyl, and heterocyclyl is optionally substituted with one or more R¹⁷; or
R^{B} and R^{C}, together with the nitrogen atom to which they are attached, form —N═C(R^{D})(R^{E}) or a heterocyclyl, wherein the heterocyclyl is optionally substituted with one or more R¹⁷;
each R^{D} and R^{E} is independently hydrogen, C₁₋₆ alkyl, C₂₋₆ alkenyl, C₁₋₆ heteroalkyl, C₁₋₆ haloalkyl, cycloalkyl, or heterocyclyl, wherein each alkyl, alkenyl, heteroalkyl, haloalkyl, cycloalkyl, and heterocyclyl is optionally substituted with one or more R¹⁷; or
each R¹⁷ is deuterium, C₁₋₆ alkyl, C₁₋₆ heteroalkyl, C₁₋₆ haloalkyl, cycloalkyl, halogen, or cyano; or
two R¹⁷ form an oxo;
each R¹⁸ is independently H, C₁₋₆ alkyl, or halo; and
n is 0, 1, 2, 3, 4, 5, or 6.

In some embodiments, the compound of Formula (I) is a compound of Formula (I′):

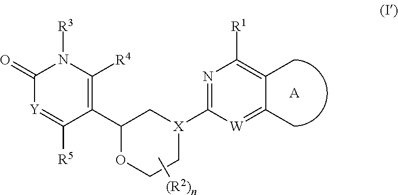

or a pharmaceutically acceptable salt, solvate, stereoisomer, or tautomer thereof, or a pharmaceutically acceptable salt and/or solvate of said compound, stereoisomer, or tautomer, wherein

Ring A, together with the 6-membered ring system to which it is fused, forms a bicyclic ring system selected from one of

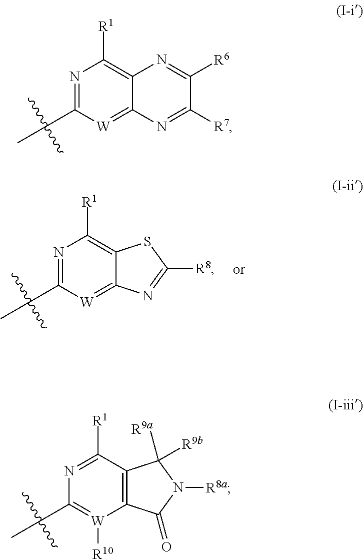

W is C(R¹⁰) or N;
X is C(R¹¹) or N;
Y is C(R¹²) or N;
R¹ is cycloalkyl, heterocyclyl, aryl, or heteroaryl, each of which is optionally substituted with one or more R¹³;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁴; or
two R², taken together with the carbon atom to which they are attached, form an oxo group;
R³ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁵;
R⁴ and R⁵ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁴;
R⁶ and R⁷ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁶;
R⁸ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, or N(R^{B})(R^{C}), wherein said alkyl, alkenyl, alkynyl, heteroalkyl, haloalkyl, cycloalkyl, and heterocyclyl is optionally substituted with one or more R¹⁶;
R^{8a} is hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein said alkyl, alkenyl, alkynyl, heteroalkyl, haloalkyl, cycloalkyl, and heterocyclyl is optionally substituted with one or more R¹⁶;
R^{9a} and R^{9b} are each independently C₁₋₆ alkyl, C₂₋₆ alkenyl, C₂₋₆ alkynyl, C₁₋₆ heteroalkyl, C₁₋₆ haloalkyl or halogen;
R¹⁰, R¹¹, and R¹² are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁶;
each R¹³ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁷;
each R¹⁴, R¹⁵, and R¹⁶ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁷;
each R^{A} is independently hydrogen, deuterium, C₁₋₆ alkyl, C₁₋₆ heteroalkyl, C₁₋₆ haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₁₋₆ heteroalkyl, C₁₋₆ haloalkyl, cycloalkyl, heterocyclyl, or —C(O)-alkyl, wherein each alkyl, heteroalkyl, haloalkyl, cycloalkyl, and heterocyclyl is optionally substituted with one or more R¹⁷;
each R¹⁷ is deuterium, C₁₋₆ alkyl, C₁₋₆ heteroalkyl, C₁₋₆ haloalkyl, cycloalkyl, halogen, or cyano; and
n is 0, 1, 2, 3, 4, 5, or 6.

In some embodiments, Ring A is

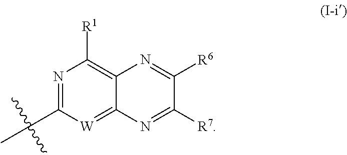

In some embodiments, W is C(R¹⁰)(e.g., CH).

In some embodiments, Ring A is

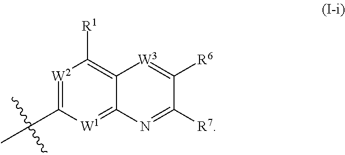

In some embodiments, W¹ is C(R¹⁰)(e.g., CH). In some embodiments, W² is C(R¹⁰)(e.g., CH). In some embodiments, W³ is C(R¹⁰) (e.g., CH). In some embodiments, W¹, W², and W³ are each N.

In some embodiments, R¹ is a 4-membered, 5-membered, or 6-membered cycloalkyl, each of which is optionally substituted with one or more R¹³. In some embodiments, R¹ is selected from cyclopropyl, cyclopentyl, or cyclohexyl, each of which is optionally substituted with one or more R¹³. In other embodiments, R¹ is selected from cyclohexyl, 4,4-difluoro-cyclohexyl, 4-fluoro-cyclohexyl, 4-fluoromethyl-cyclohexyl, (spiro)-cyclopropyl-cyclopropyl-F₂, bicyclo[1.1.1]-trifluoromethyl, bicyclo[1.1.1]-difluoromethyl, bicyclo[1.1.1]-CF—(CH₃)₂, bicyclo[1.1.1]-CH₂—CF₃, bicyclo[1.1.1]-CH₂—CHF₂, bicyclo[1.1.1]-CF₂—CH₃, bicyclo[1.1.1]-chloro, or bicyclo[2.2.1]-CF₂—CH₃. In some embodiments, R¹ is aryl or heteroaryl, each of which is optionally substituted with one or more R¹³. In some embodiments, R¹ is phenyl or thiazolyl, each of which is optionally substituted with one or more R¹³. In some embodiments, R¹³ is deuterium, C₁₋₆ alkyl, C₁₋₆ heteroalkyl, C₁₋₆ haloalkyl, or halogen.

In some embodiments, R¹ is selected from those shown below:

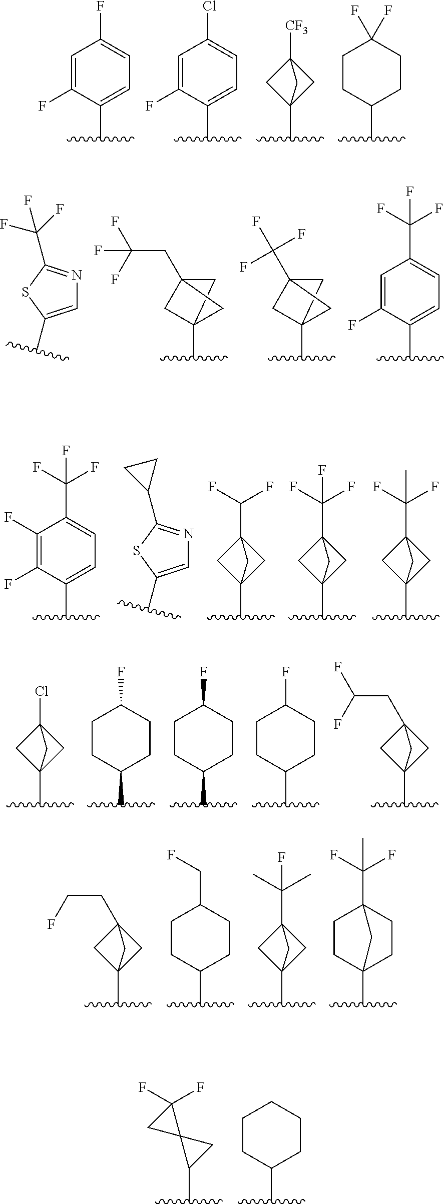

In some embodiments, R¹ is —CH₂CH₂CF₃,

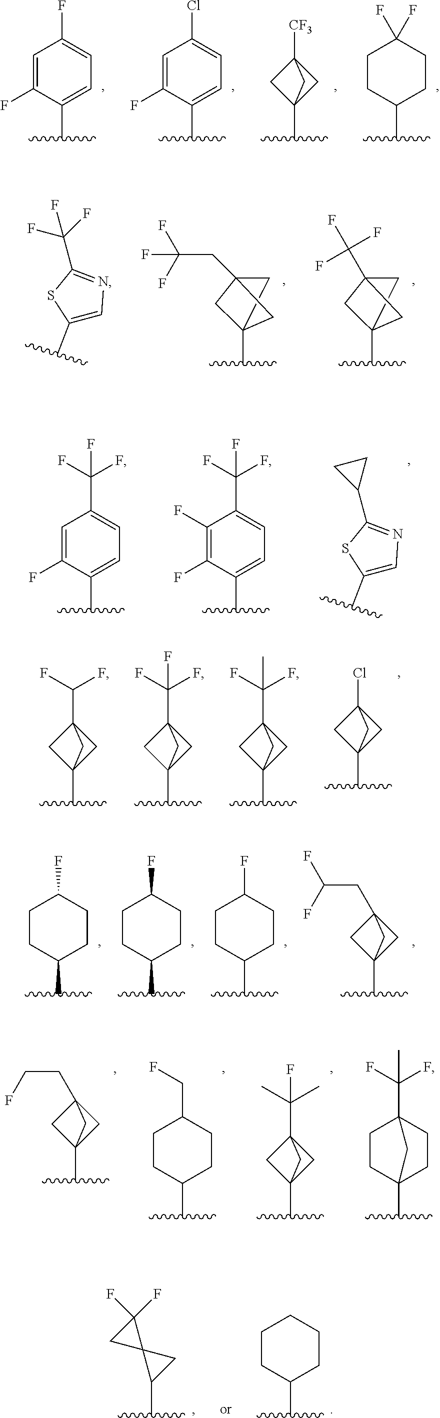

In some embodiments, R¹ is

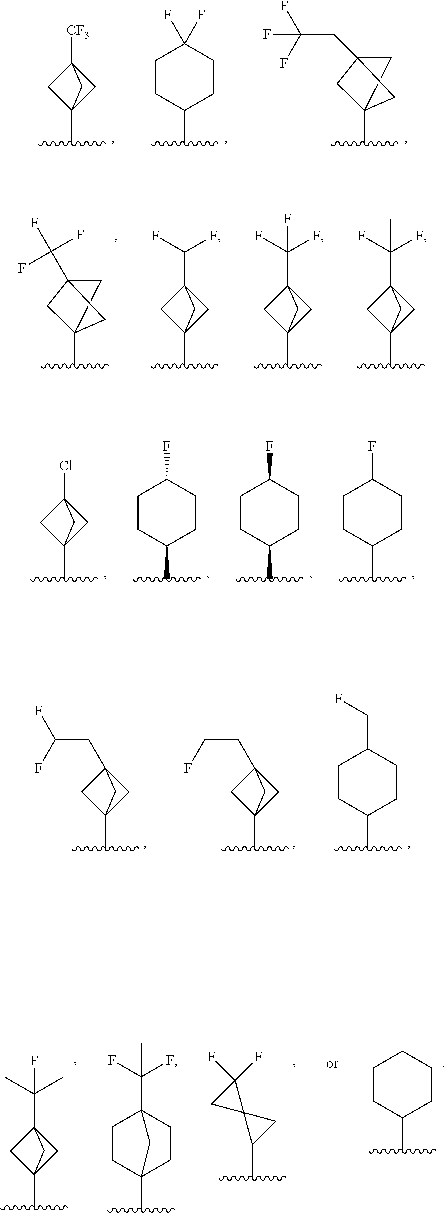

In some embodiments, R¹ is

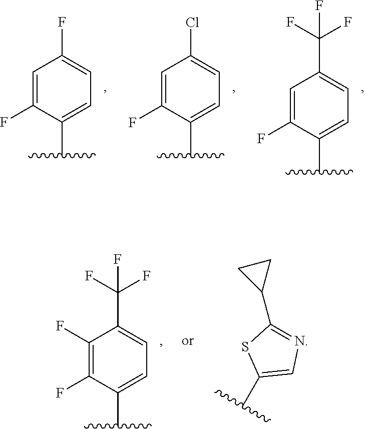

In some embodiments, R¹ is selected from cycloalkyl or heterocyclyl, each of which is optionally substituted with one or more R¹³. In some embodiments, R¹ is a 3-membered, 4-membered, 5-membered, 6-membered, 7-membered, or 8-membered cycloalkyl or heterocyclyl, each of which is optionally substituted with optionally substituted with one or more R¹³. In some embodiments, R¹ is a 3-membered, 4-membered, 5-membered, 6-membered, 7-membered, or 8-membered cycloalkyl, each of which is optionally substituted with one or more R¹³. In some embodiments, R¹ is selected from cyclopropyl, cyclobutyl, cyclopentyl, or cyclohexyl, each of which is optionally substituted with optionally substituted with one or more R¹³. In some embodiments, R¹ is selected from

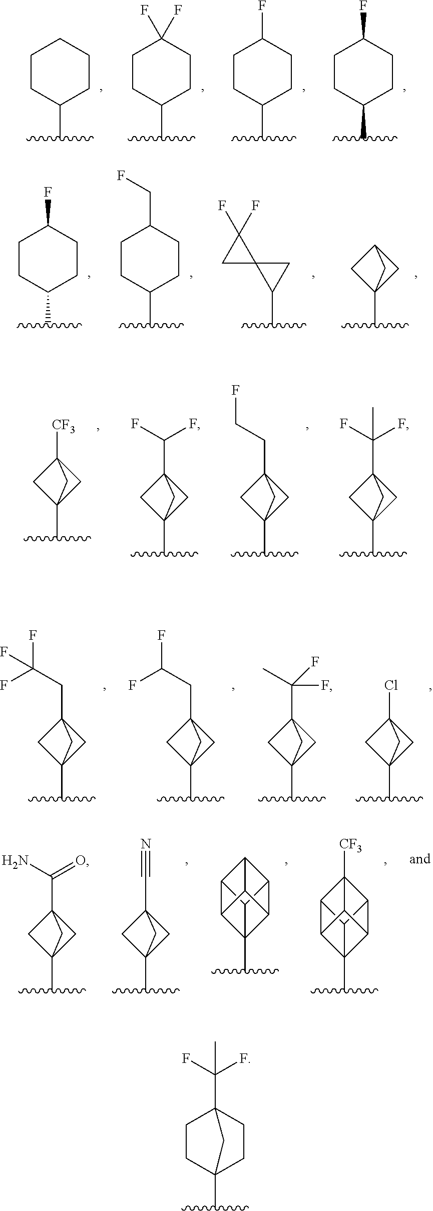

In some embodiments, R¹ is —O(R^{A}), aryl, or heteroaryl, wherein aryl and heteroaryl are each optionally substituted with one or more R¹³. In some embodiments, R¹ is selected from phenyl, pyridyl, pyrimidyl, imidazolyl, oxadiaxolyl, isoxazolyl, oxazoleyl, isothiazolyl, thiazolyl, pyrazolyl, or pyrazyl, each of which is optionally substituted with one or more R¹³. In some embodiments, R¹ is phenyl or thiazolyl, each of which is optionally substituted with one or more R¹³. In some embodiments, R¹ is selected from

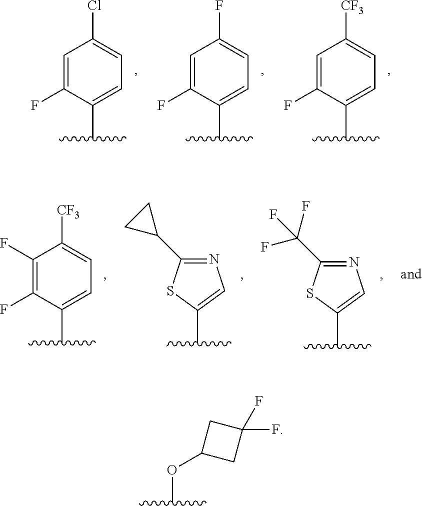

In some embodiments, R¹³ is deuterium, C₁₋₆ alkyl, C₁₋₆ heteroalkyl, cycloalkyl, C₁₋₆ haloalkyl, halogen, cyano, or —C(O)N(R^{B})(R^{C}).

In some embodiments, R¹ is selected from those depicted in Table A below.

In some embodiments, R⁶ and R⁷ are each independently hydrogen, C₁₋₆ alkyl, C₁₋₆ heteroalkyl, C₁₋₆ haloalkyl, cycloalkyl, heterocyclyl, halogen, cyano, or —O(R^{A}), wherein each alkyl, heteroalkyl, haloalkyl, cycloalkyl, and heterocyclyl is optionally substituted with one or more R¹⁶. In some embodiments, R⁶ and R⁷ are each independently C₁₋₆ alkyl (e.g., CH₃). In some embodiments, one of R⁶ and R⁷ is independently C₁₋₆ alkyl (e.g., CH₃) and the other of R⁶ and R⁷ is independently-O(R^{A}) (e.g., CH₃). In some embodiments, one of R⁶ and R⁷ is selected from those depicted in Table A below.

In some embodiments, R⁶ and R⁷ are each independently hydrogen, C₁₋₆ alkyl, C₁₋₆ heteroalkyl, C₁₋₆ haloalkyl, cycloalkyl, heterocyclyl, aryl, heteroaryl, halogen, cyano, or —O(R^{A}), wherein each alkyl, heteroalkyl, haloalkyl, cycloalkyl, heterocyclyl, aryl, and heteroaryl is optionally substituted with one or more R¹⁶. In some embodiments, one of R⁶ and R⁷ is independently C₁₋₆ alkyl (e.g., CH₃) and the other of R⁶ and R⁷ is independently C₁₋₆ alkyl or —O(R^{A})(e.g., OCH₃). In some embodiments, one of R⁶ and R⁷ is CH₃, and the other of R⁶ and R⁷ is OCH₃. In some embodiments, R⁶ and R⁷ are each independently selected from

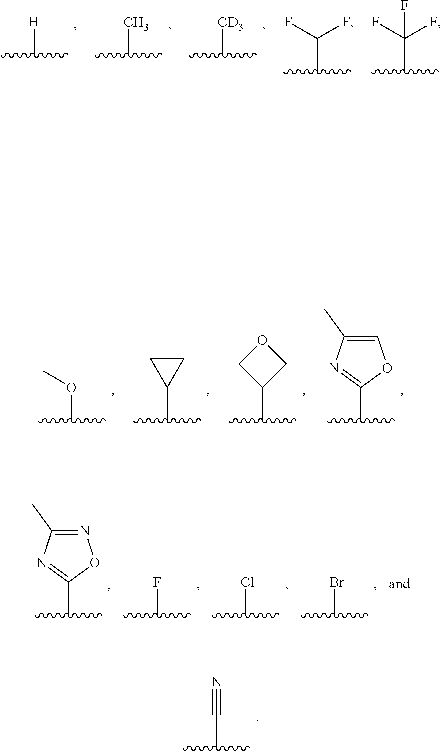

In some embodiments, X is C(R¹¹)(e.g., CH₃). In some embodiments, X is N. In some embodiments, X is selected from those depicted in Table A below.

In some embodiments, X is C(R¹¹)(e.g., CH).

In some embodiments, Y is C(R¹²). In some embodiments, Y is N. In some embodiments, Y is selected from those depicted in Table A below.

In some embodiments, R¹² is hydrogen, C₁₋₆ alkyl, C₁₋₆ heteroalkyl, C₁₋₆ haloalkyl, cycloalkyl, heterocyclyl, heteroaryl, halogen, cyano, or —N(R^{B})(R^{C}), wherein each alkyl, heteroalkyl, haloalkyl, cycloalkyl, and heterocyclyl is optionally substituted with one or more R¹⁶. In some embodiments, R¹² is C₁₋₆ alkyl optionally substituted with one or more R¹⁶ (e.g., CH₃, CD₃). In some embodiments, R¹² is C₁₋₆ haloalkyl (e.g., CHF₂, CF₃). In some embodiments, R¹² is cycloalkyl, heterocyclyl, or heteroaryl optionally substituted with one or more R¹⁶. In some embodiments, R¹² is selected from those depicted in Table A below.

In some embodiments, Z is C(R¹⁸)₂. In some embodiments, R¹⁸ is hydrogen or halogen. In some embodiments, R¹⁸ is F. In some embodiments, Z is O.

In some embodiments, R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁴; or two R², taken together with the carbon atom to which they are attached, form an oxo group. In some embodiments, R² is selected from those depicted in Table A below.

In some embodiments, R³ is selected from those shown below:

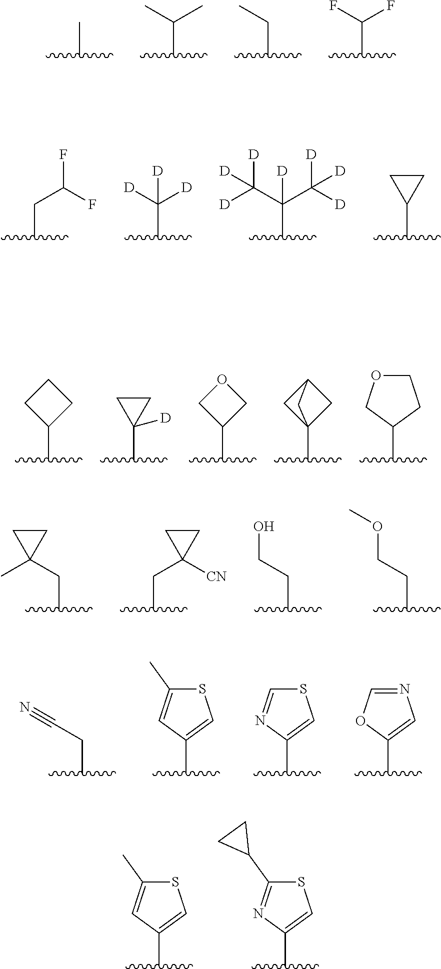

In some embodiments, R³ is substituted with C₁₋₃alkyl comprising one or more deuteriums. In some embodiments, R³ is substituted with 1 to 3 substitutents selected from -CD₃, -CHD₂, and —CH₂D.

In some embodiments, R³ is hydrogen, C₁₋₆ alkyl, C₁₋₆ heteroalkyl, C₁₋₆ haloalkyl, cycloalkyl, heterocyclyl, aryl, heteroaryl, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁵. In some embodiments, R³ is selected from hydrogen, Cl,

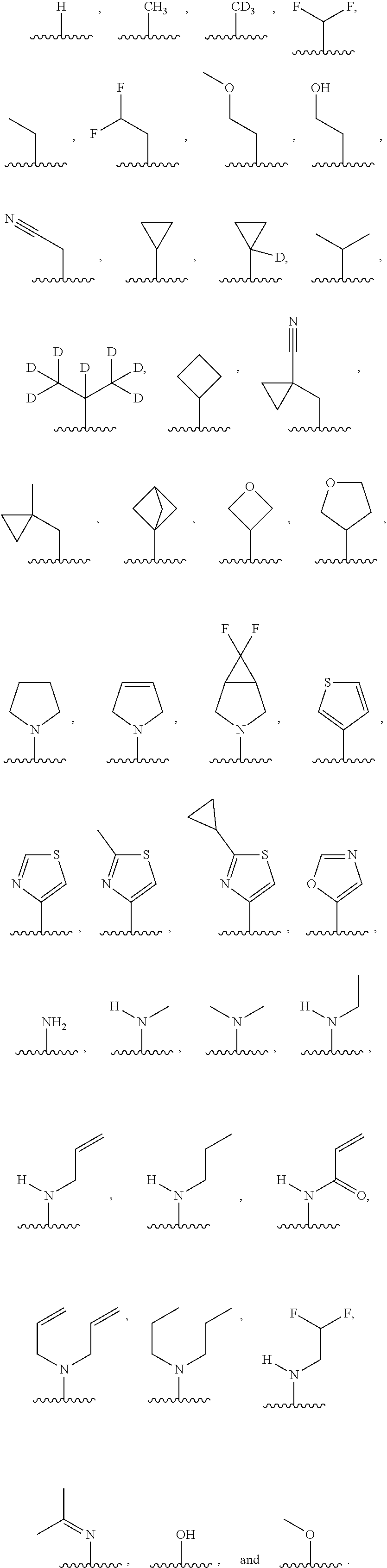

In some embodiments, R⁴ and R⁵ are each independently hydrogen or C₁₋₆ alkyl.

In some embodiments, R⁴ is selected from those depicted in Table A below.

In some embodiments, R⁴ and R⁵ are each independently hydrogen or C₁₋₆ alkyl. In some embodiments, one of R⁴ and R⁵ is selected from those depicted in Table A below.

In some embodiments, n is 0 or 1.

In some embodiments, n is 0. In some embodiments, n is 1.

In some embodiments, Ring A is

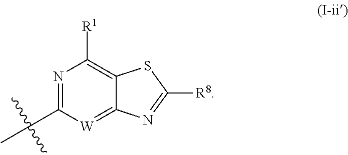

In some embodiments, W is C(R¹⁰) or N, R⁸ is C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein said alkyl, alkenyl, alkynyl, heteroalkyl, haloalkyl, cycloalkyl, or heterocyclyl is optionally substituted with one or more R¹⁶.

In some embodiments, Ring A is

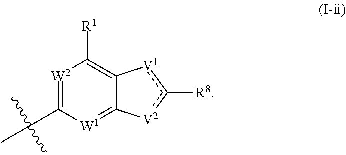

In some embodiments, V¹ is S and V² is N. In some embodiments, V¹ is NR^{8b} and V² is N. In some embodiments, V¹ is N and V² is NR⁸b. In some embodiments, W¹ and W² are each independently N. In some embodiments, R⁸ is C₁₋₆ alkyl, C₂₋₆ alkenyl, C₂₋₆ alkynyl, C₁₋₆ heteroalkyl, C₁₋₆ haloalkyl, cycloalkyl, heterocyclyl, or —N(R^{B})(R^{C}), wherein alkyl, alkenyl, alkynyl, heteroalkyl, haloalkyl, cycloalkyl, and heterocyclyl is optionally substituted with one or more R¹⁶. In some embodiments, R⁸ is —N(R^{B})(R^{C}). In some embodiments, R^{B} and R^{C} are each independently C₁₋₆ alkyl. In some embodiments, R^{B} and R^{C} are each independently CH₃. In some embodiments, R^{8b} is C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶. In some embodiments, R^{8b} is C₁₋₆ alkyl. In some embodiments, R^{8b} is CH₃.

In some embodiments, Ring A is

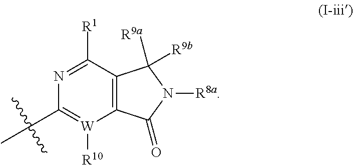

In some embodiments, W is C(R¹⁰) or N, R^{8a} is hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein said alkyl, alkenyl, alkynyl, heteroalkyl, haloalkyl, cycloalkyl, or heterocyclyl is optionally substituted with one or more R¹⁶. In some embodiments, each R⁹ª and Rob is hydrogen.

In some embodiments, Ring A together with the 6-membered ring system to which it is fused forms

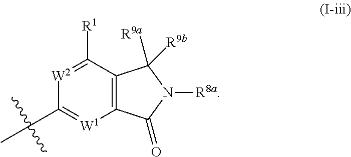

In some embodiments, R^{8a} is hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶. In some embodiments, R^{8a} is C₁₋₆ alkyl. In some embodiments, R^{8a} is CH₃. In some embodiments, each of R^{9a} and R^{9b} is hydrogen.

In some embodiments, Ring A together with the 6-membered ring system to which it is fused is selected from

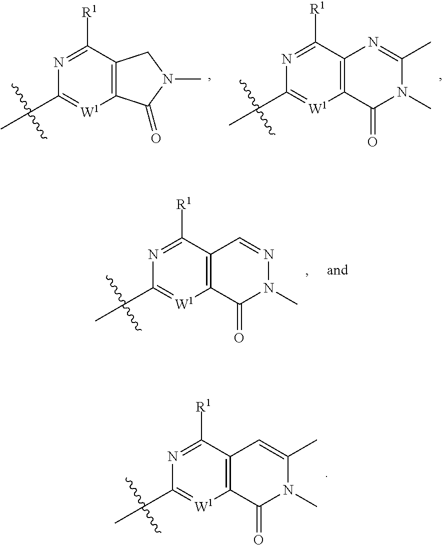

In some embodiments, the compound of Formula (I) is a compound of Formula (I-a)

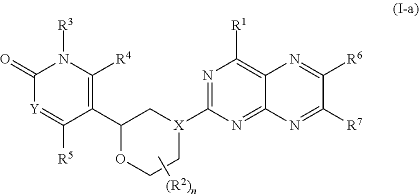

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴. R⁵, R⁶, R⁷, n and subvariables thereof are defined as for Formula (I).

In some embodiments, the compound of Formula (I) is a compound of Formula (I-b)

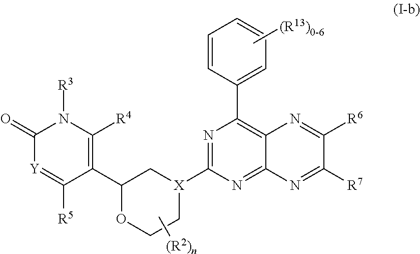

or a pharmaceutically acceptable salt, solvate, stereoisomer, or tautomer thereof, or a pharmaceutically acceptable salt and/or solvate of said compound, stereoisomer, or tautomer, wherein each of X, R², R³, R⁴. R⁵, R⁶, R⁷, R¹³, n and subvariables thereof are defined as for Formula (I).

In some embodiments, the compound of Formula (I) is a compound of Formula (I-c)

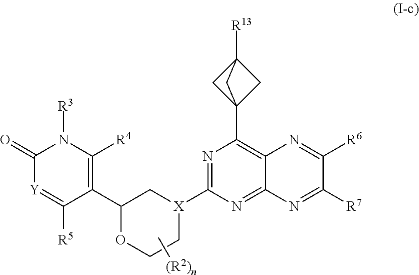

or a pharmaceutically acceptable salt, solvate, stereoisomer, or tautomer thereof, or a pharmaceutically acceptable salt and/or solvate of said compound, stereoisomer, or tautomer, wherein each of X, R², R³, R⁴. R⁵, R⁶, R⁷, R¹³, n and subvariables thereof are defined as for Formula (I).

In some embodiments, the compound of Formula (I) is a compound of Formula (I-d)

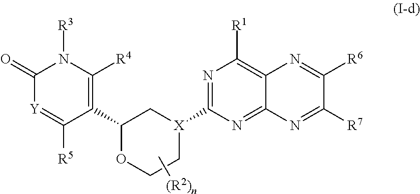

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴. R⁵, R⁶, R⁷, n and subvariables thereof are defined as for Formula (I).

In some embodiments, the compound of Formula (I) is a compound of Formula (I-e)

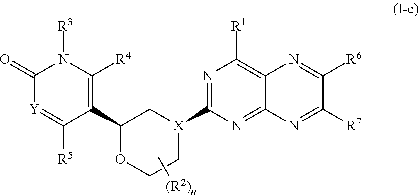

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴. R⁵, R⁶, R⁷, n and subvariables thereof are defined as for Formula (I).

In some embodiments, the compound of Formula (I) is a compound of Formula (I-f)

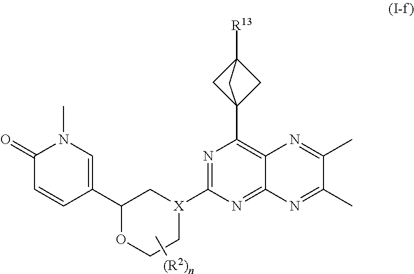

or a pharmaceutically acceptable salt, solvate, stereoisomer, or tautomer thereof, or a pharmaceutically acceptable salt and/or solvate of said compound, stereoisomer, or tautomer, wherein each of X, R², R¹³, n and subvariables thereof are defined as for Formula (I).

In some embodiments, the compound of Formula (I) is a compound of Formula (I-g)

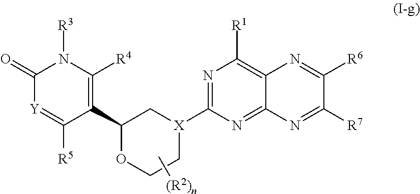

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴, R⁵, R⁶, R⁷, n and subvariables thereof are defined as for Formula (I).

In some embodiments, the compound of Formula (I) is a compound of Formula (I-h)

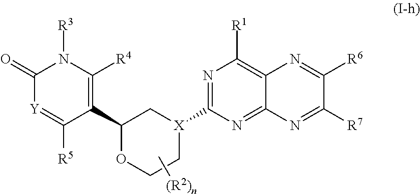

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴, R⁵, R⁶, R⁷, n and subvariables thereof are defined as for Formula (I).

In some embodiments, the compound of Formula (I) is a compound of Formula (I-j)

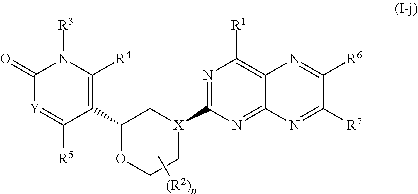

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴, R⁵, R⁶, R⁷, n and subvariables thereof are defined as for Formula (I).

In some embodiments, the compound of Formula (I) is a compound of Formula (I-k)

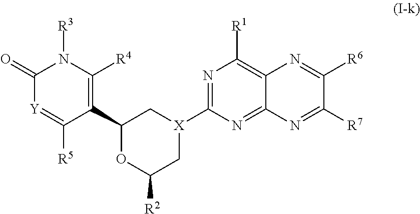

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴, R⁵, R⁶, R⁷, and subvariables thereof are defined as for Formula (I).

In some embodiments, the compound of Formula (I) is a compound of Formula (I-1)

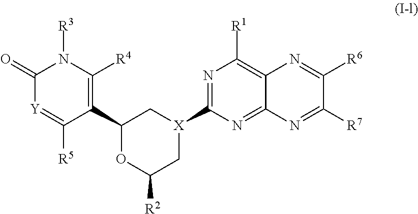

or a pharmaceutically acceptable salt, solvate, stereoisomer, or tautomer thereof, or a pharmaceutically acceptable salt and/or solvate of said compound, stereoisomer, or tautomer, wherein each of X, R¹, R², R³, R⁴, R⁵, R⁶, R⁷, and subvariables thereof are defined as for Formula (I).

In some embodiments, the compound of Formula (I) is a compound of Formula (I-m)

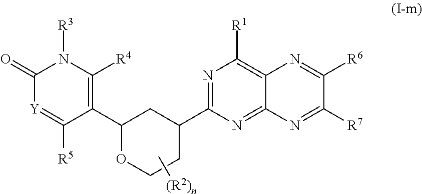

r a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴. R⁵, R⁶, R⁷, n and subvariables thereof are defined as for Formula (I).

In some embodiments, the compound of Formula (I) is a compound of Formula (I-n)

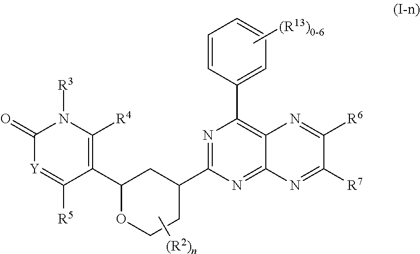

or a pharmaceutically acceptable salt, solvate, stereoisomer, or tautomer thereof, or a pharmaceutically acceptable salt and/or solvate of said compound, stereoisomer, or tautomer, wherein each of R², R³, R⁴. R⁵, R⁶, R⁷, R¹³, n and subvariables thereof are defined as for Formula (I).

In some embodiments, the compound of Formula (I) is a compound of Formula (I-o)

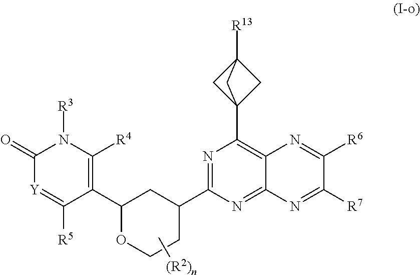

or a pharmaceutically acceptable salt, solvate, stereoisomer, or tautomer thereof, or a pharmaceutically acceptable salt and/or solvate of said compound, stereoisomer, or tautomer, wherein each of R², R³, R⁴. R⁵, R⁶, R⁷, R¹³, n and subvariables thereof are defined as for Formula (I).

In some embodiments, the compound of Formula (I) is a compound of Formula (I-p)

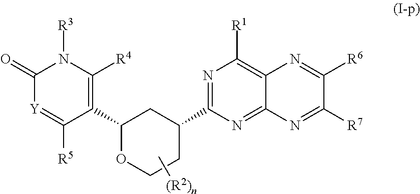

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴. R⁵, R⁶, R⁷, n and subvariables thereof are defined as for Formula (I).

In some embodiments, the compound of Formula (I) is a compound of Formula (I-q)

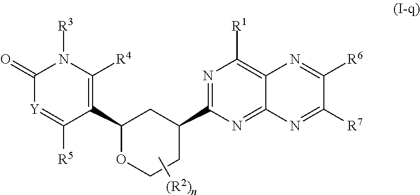

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴. R⁵, R⁶, R⁷, n and subvariables thereof are defined as for Formula (I).

In some embodiments, the compound of Formula (I) is a compound of Formula (I-r)

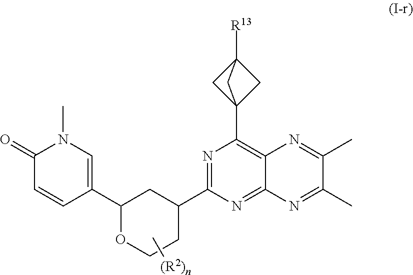

or a pharmaceutically acceptable salt, solvate, stereoisomer, or tautomer thereof, or a pharmaceutically acceptable salt and/or solvate of said compound, stereoisomer, or tautomer, wherein each of R², R¹³, n and subvariables thereof are defined as for Formula (I).

In some embodiments, the compound of Formula (I) is a compound of Formula (I-s)

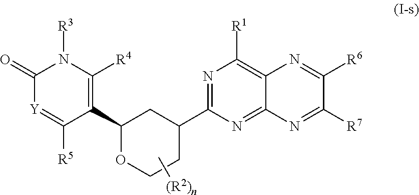

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴, R⁵, R⁶, R⁷, n and subvariables thereof are defined as for Formula (I).

In some embodiments, the compound of Formula (I) is a compound of Formula (I-t)

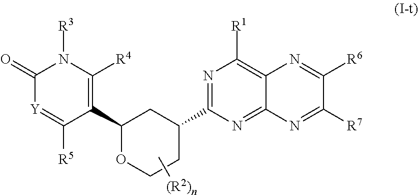

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴, R⁵, R⁶, R⁷, n and subvariables thereof are defined as for Formula (I).

In some embodiments, the compound of Formula (I) is a compound of Formula (I-u)

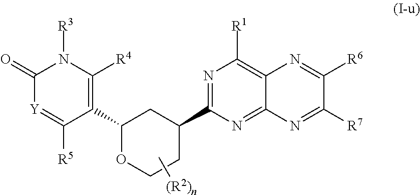

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴, R⁵, R⁶, R⁷, n and subvariables thereof are defined as for Formula (I).

In some embodiments, the compound of Formula (I) is a compound of Formula (I-v)

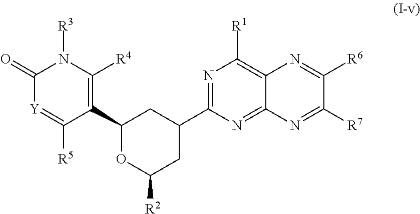

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴, R⁵, R⁶, R⁷, n and subvariables thereof are defined as for Formula (I).

In some embodiments, the compound of Formula (I) is a compound of Formula (I-w)

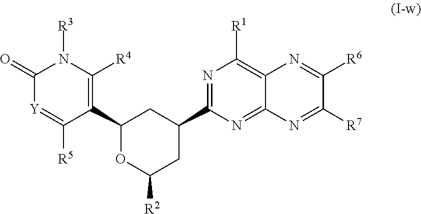

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R⁴, R⁵, R⁶, R⁷, n and subvariables thereof are defined as for Formula (I).

In some embodiments, the compound of Formula (I) is a compound of Formula (I-x):

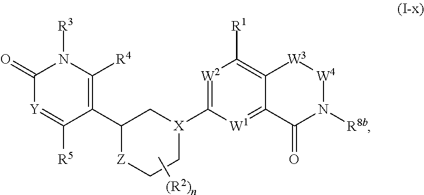

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R⁴, R⁵, R^{8b}, W¹, W², W³, W⁴, n and subvariables thereof are defined as for Formula (I).

In some embodiments, the compound of Formula (I) is a compound of Formula (I-y):

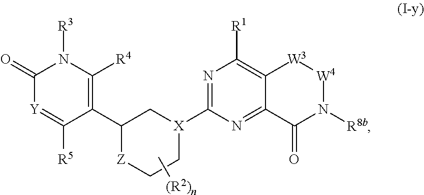

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R⁴, R⁵, R^{8b}, W¹, W², W³, W⁴, n and subvariables thereof are defined as for Formula (I).

In some embodiments, the compound of Formula (I) is a compound of Formula (I-z):

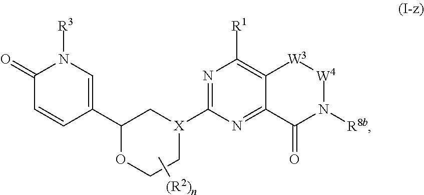

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R⁴, R⁵, R^{8b}, W³, W⁴, n and subvariables thereof are defined as for Formula (I).

In some embodiments, the compound of Formula (I) is a compound of Formula (I-aa):

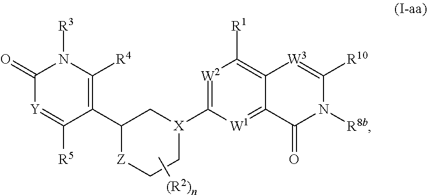

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R⁴, R⁵, R^{8b}, W¹, W², W³, n and subvariables thereof are defined as for Formula (I).

In some embodiments, the compound of Formula (I) is a compound of Formula (I-bb):

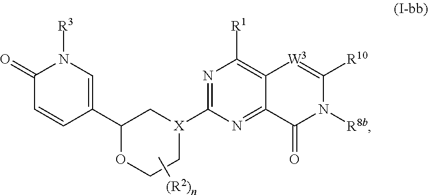

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R⁴, R⁵, R^{8b}, W³, n and subvariables thereof are defined as for Formula (I).

In some embodiments, the compound of Formula (I) is a compound of Formula (I-cc):

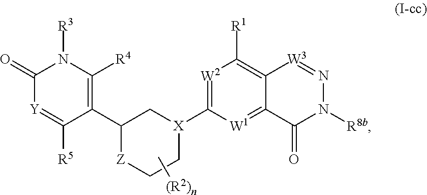

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R⁴, R⁵, R^{8b}, W¹, W², W³, n and subvariables thereof are defined as for Formula (I).

In some embodiments, the compound of Formula (I) is a compound of Formula (I-dd):

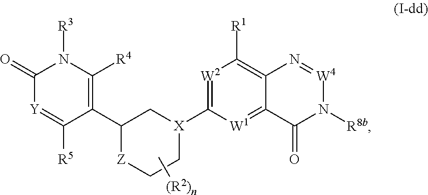

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R⁴, R⁵, R^{8b}, W¹, W², W⁴, n and subvariables thereof are defined as for Formula (I).

In some embodiments, the compound of Formula (I) is a compound of Formula (I-ee):

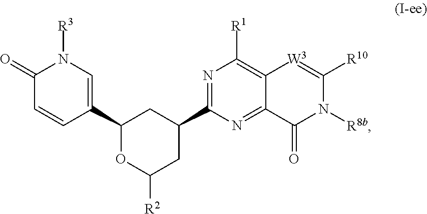

or a pharmaceutically acceptable salt, solvate, stereoisomer, or tautomer thereof, or a pharmaceutically acceptable salt and/or solvate of said compound, stereoisomer, or tautomer, wherein each of R¹, R², R³, R^{8b}, R¹⁰, W³, n and subvariables thereof are defined as for Formula (I).

In some embodiments, the compound of Formula (I) is a compound of Formula (I-ff):

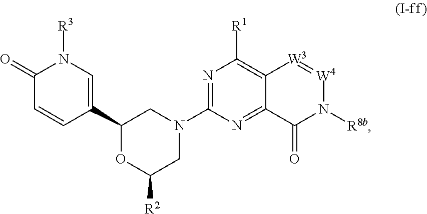

or a pharmaceutically acceptable salt, solvate, stereoisomer, or tautomer thereof, or a pharmaceutically acceptable salt and/or solvate of said compound, stereoisomer, or tautomer, wherein each of X, Y, Z, R¹, R², R³, R^{8b}, W³, W⁴, n and subvariables thereof are defined as for Formula (I).

In some embodiments, the compound of Formula (I) is a compound of Formula (I-gg):

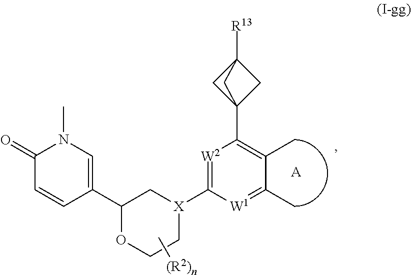

or a pharmaceutically acceptable salt, solvate, stereoisomer, or tautomer thereof, or a pharmaceutically acceptable salt and/or solvate of said compound, stereoisomer, or tautomer, wherein each of Ring A, W¹, W², R², R¹³, n and subvariables thereof are defined as for Formula (I).

In some embodiments, the compound of Formula (I) is a compound provided in Table A.

Further provided herein is a pharmaceutical composition comprising one or more of the compounds of Formula (I), a pharmaceutically acceptable salt, solvate, stereoisomer, or tautomer thereof, or a pharmaceutically acceptable salt and/or solvate of said compound, stereoisomer, or tautomer disclosed herein and a pharmaceutically acceptable excipient.

In some embodiments, the compound is a compound of any of the previous embodiments, or a pharmaceutical composition of the previous embodiments, for use in treating or preventing a condition associated with a loss of function of human TREM2.

Further provided herein is a method of treating or preventing a condition associated with a loss of function of human TREM2 in a subject in need thereof, the method comprising administering to the subject a therapeutically effective amount of the compound according to any one of the previous embodiments, or a pharmaceutical composition of the previous embodiments.

In some embodiments, at least one hydrogen atom of the compound is a deuterium atom. In some embodiments, at least one C₁-C₆alkyl group of the compound is substituted with at least one deuterium atom. In some embodiments, R⁶ is -CD₃. In some embodiments, R⁷ is -CD₃. In some embodiments, R⁶ and R⁷ are both -CD₃. In some embodiments, R⁶ and R⁷ are each independently selected from H, D, —CH₃, -CD₃, -CHD₂, and -CH₂D. In some embodiments, R⁶ and R⁷ are each independently selected from —CH₃, -CD₃, -CHD₂, and -CH₂D. In some embodiments, R² is deuterium.

In some embodiments, the hydrogen atom attached to the same carbon as R² is deuterium. In some embodiments, R³ is substituted with C₁₋₃alkyl, comprising one or more deuteriums. In some embodiments, R³ is substituted with 1 to 3 substitutents selected from -CD₃, -CHD₂, and -CH₂D.

Exemplary compounds of the disclosure are set forth in Table A, below. In some embodiments, the compound is a compound set forth in Table A, or a pharmaceutically acceptable salt, solvate, stereoisomer, or tautomer thereof, or a pharmaceutically acceptable salt and/or solvate of said compound, stereoisomer, or tautomer thereof.

TABLE A
Exemplary Compounds

| Ex | |
|---|---|
| # | Structure |
| 1 | 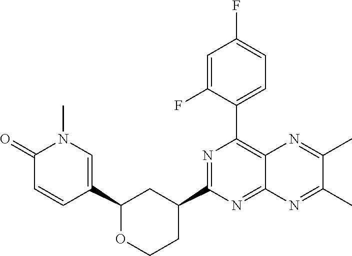 [SMILES: Cc1nc2nc([C@H]3CCO[C@@H](c4ccc(=O)n(C)c4)C3)nc(-c3ccc(F)cc3F)c2nc1C] |
| 2 | 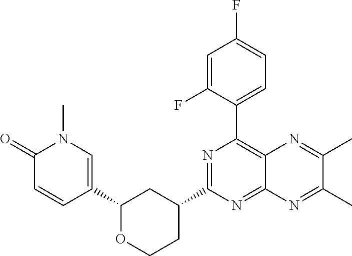 [SMILES: Cc1nc2nc([C@@H]3CCO[C@H](c4ccc(=O)n(C)c4)C3)nc(-c3ccc(F)cc3F)c2nc1C] |
| 3 | 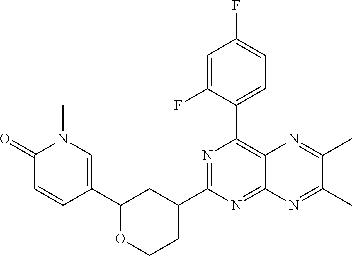 [SMILES: Cc1nc2nc(C3CCOC(c4ccc(=O)n(C)c4)C3)nc(-c3ccc(F)cc3F)c2nc1C] |
| 4 | 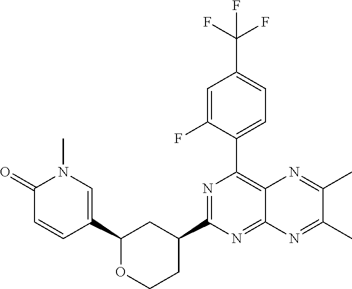 [SMILES: Cc1nc2nc([C@H]3CCO[C@@H](c4ccc(=O)n(C)c4)C3)nc(-c3ccc(C(F)(F)F)cc3F)c2nc1C] |
| 5 | 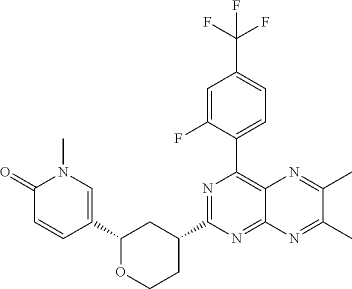 [SMILES: Cc1nc2nc([C@@H]3CCO[C@H](c4ccc(=O)n(C)c4)C3)nc(-c3ccc(C(F)(F)F)cc3F)c2nc1C] |
| 6 | 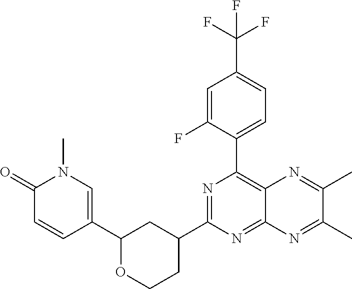 [SMILES: Cc1nc2nc(C3CCOC(c4ccc(=O)n(C)c4)C3)nc(-c3ccc(C(F)(F)F)cc3F)c2nc1C] |
| 7 | 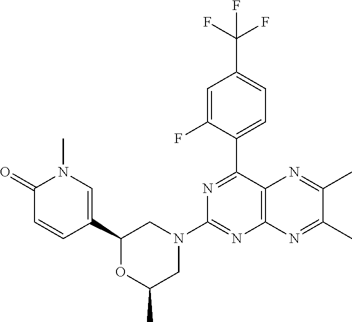 [SMILES: Cc1nc2nc(N3C[C@@H](C)O[C@@H](c4ccc(=O)n(C)c4)C3)nc(-c3ccc(C(F)(F)F)cc3F)c2nc1C] |
| 8 | 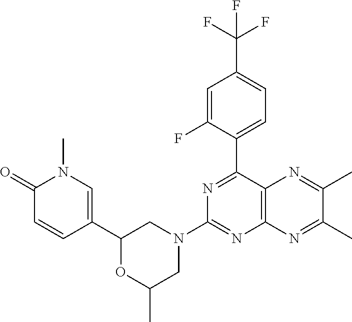 [SMILES: Cc1nc2nc(N3CC(C)OC(c4ccc(=O)n(C)c4)C3)nc(-c3ccc(C(F)(F)F)cc3F)c2nc1C] |
| 9 | 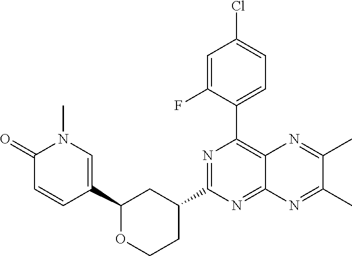 [SMILES: Cc1nc2nc([C@@H]3CCO[C@@H](c4ccc(=O)n(C)c4)C3)nc(-c3ccc(Cl)cc3F)c2nc1C] |
| 10 | 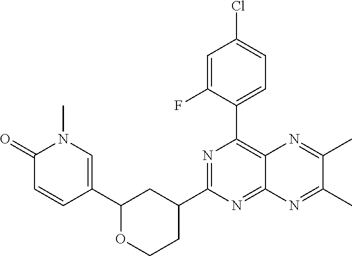 [SMILES: Cc1nc2nc(C3CCOC(c4ccc(=O)n(C)c4)C3)nc(-c3ccc(Cl)cc3F)c2nc1C] |
| 11 | 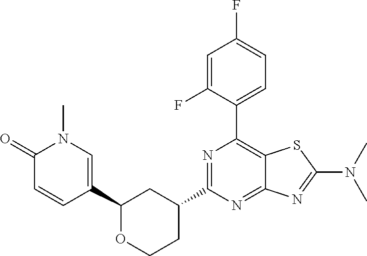 [SMILES: CN(C)c1nc2nc([C@@H]3CCO[C@@H](c4ccc(=O)n(C)c4)C3)nc(-c3ccc(F)cc3F)c2s1] |
| 12 | 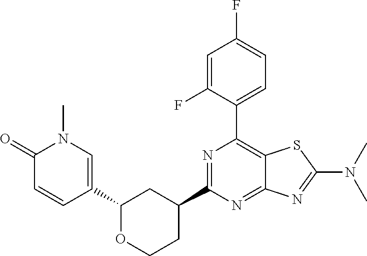 [SMILES: CN(C)c1nc2nc([C@H]3CCO[C@H](c4ccc(=O)n(C)c4)C3)nc(-c3ccc(F)cc3F)c2s1] |
| 13 | 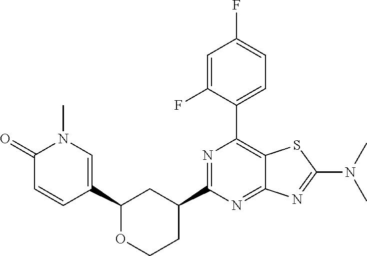 [SMILES: CN(C)c1nc2nc([C@H]3CCO[C@@H](c4ccc(=O)n(C)c4)C3)nc(-c3ccc(F)cc3F)c2s1] |
| 14 | 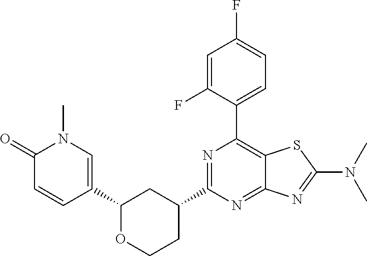 [SMILES: CN(C)c1nc2nc([C@@H]3CCO[C@H](c4ccc(=O)n(C)c4)C3)nc(-c3ccc(F)cc3F)c2s1] |
| 15 | 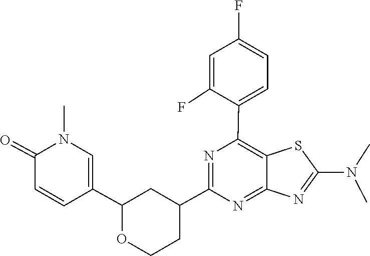 [SMILES: CN(C)c1nc2nc(C3CCOC(c4ccc(=O)n(C)c4)C3)nc(-c3ccc(F)cc3F)c2s1] |
| 16 | 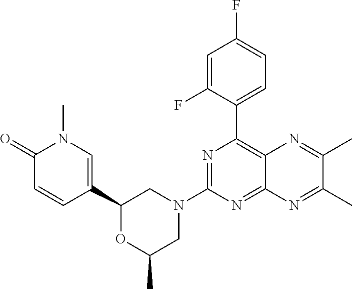 [SMILES: Cc1nc2nc(N3C[C@@H](C)O[C@@H](c4ccc(=O)n(C)c4)C3)nc(-c3ccc(F)cc3F)c2nc1C] |
| 17 | 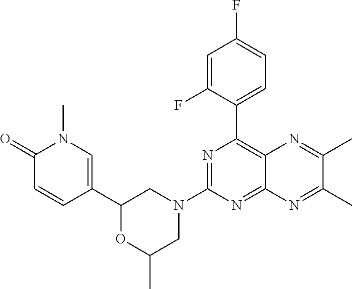 [SMILES: Cc1nc2nc(N3CC(C)OC(c4ccc(=O)n(C)c4)C3)nc(-c3ccc(F)cc3F)c2nc1C] |
| 18 | 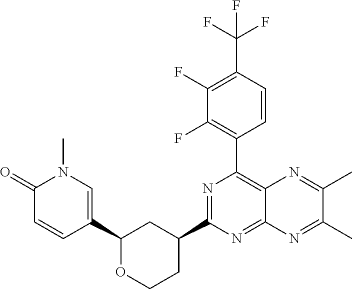 [SMILES: Cc1nc2nc([C@H]3CCO[C@@H](c4ccc(=O)n(C)c4)C3)nc(-c3ccc(C(F)(F)F)c(F)c3F)c2nc1C] |
| 19 | 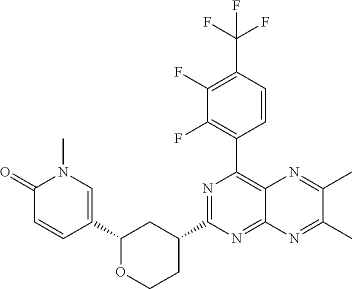 [SMILES: Cc1nc2nc([C@@H]3CCO[C@H](c4ccc(=O)n(C)c4)C3)nc(-c3ccc(C(F)(F)F)c(F)c3F)c2nc1C] |
| 20 | 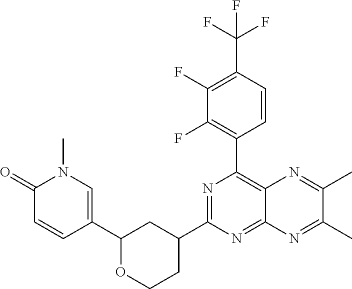 [SMILES: Cc1nc2nc(C3CCOC(c4ccc(=O)n(C)c4)C3)nc(-c3ccc(C(F)(F)F)c(F)c3F)c2nc1C] |
| 21 | 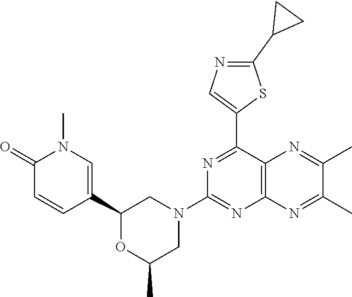 [SMILES: Cc1nc2nc(N3C[C@@H](C)O[C@@H](c4ccc(=O)n(C)c4)C3)nc(-c3cnc(C4CC4)s3)c2nc1C] |
| 22 | 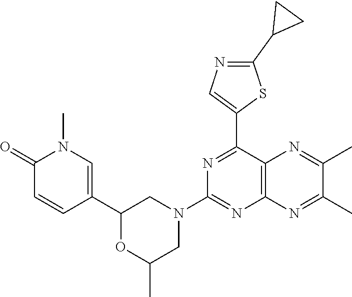 [SMILES: Cc1nc2nc(N3CC(C)OC(c4ccc(=O)n(C)c4)C3)nc(-c3cnc(C4CC4)s3)c2nc1C] |
| 23 | 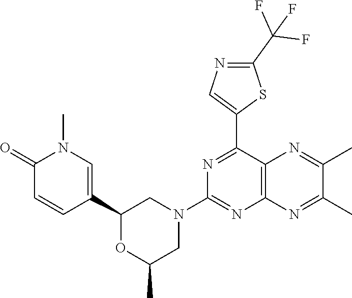 [SMILES: Cc1nc2nc(N3C[C@@H](C)O[C@@H](c4ccc(=O)n(C)c4)C3)nc(-c3cnc(C(F)(F)F)s3)c2nc1C] |
| 24 | 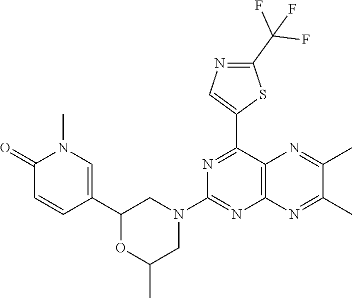 [SMILES: Cc1nc2nc(N3CC(C)OC(c4ccc(=O)n(C)c4)C3)nc(-c3cnc(C(F)(F)F)s3)c2nc1C] |
| 25 | 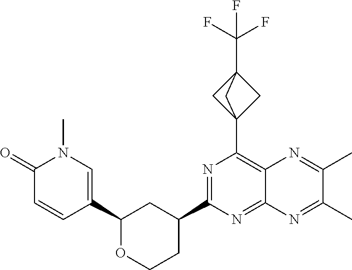 [SMILES: Cc1nc2nc([C@H]3CCO[C@@H](c4ccc(=O)n(C)c4)C3)nc(C34CC(C(F)(F)F)(C3)C4)c2nc1C] |
| 26 | 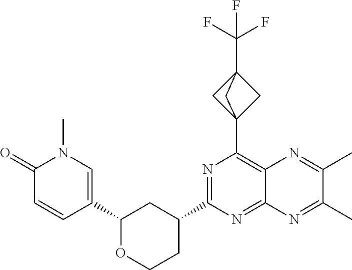 [SMILES: Cc1nc2nc([C@@H]3CCO[C@H](c4ccc(=O)n(C)c4)C3)nc(C34CC(C(F)(F)F)(C3)C4)c2nc1C] |
| 27 | 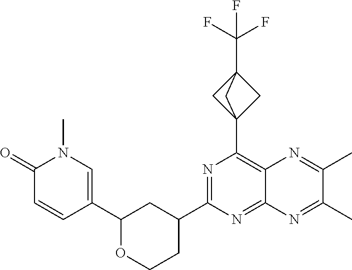 [SMILES: Cc1nc2nc(C3CCOC(c4ccc(=O)n(C)c4)C3)nc(C34CC(C(F)(F)F)(C3)C4)c2nc1C] |
| 28 | 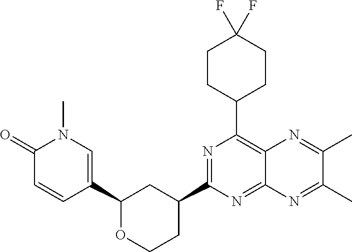 [SMILES: Cc1nc2nc([C@H]3CCO[C@@H](c4ccc(=O)n(C)c4)C3)nc(C3CCC(F)(F)CC3)c2nc1C] |
| 29 | 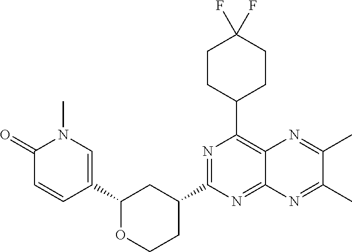 [SMILES: Cc1nc2nc([C@@H]3CCO[C@H](c4ccc(=O)n(C)c4)C3)nc(C3CCC(F)(F)CC3)c2nc1C] |
| 30 | 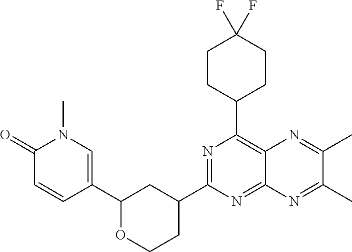 [SMILES: Cc1nc2nc(C3CCOC(c4ccc(=O)n(C)c4)C3)nc(C3CCC(F)(F)CC3)c2nc1C] |
| 31 | 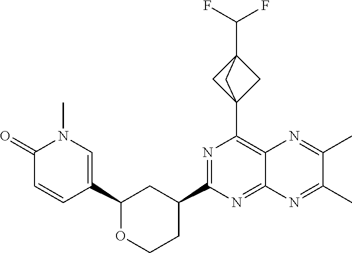 [SMILES: Cc1nc2nc([C@H]3CCO[C@@H](c4ccc(=O)n(C)c4)C3)nc(C34CC(C(F)F)(C3)C4)c2nc1C] |
| 32 | 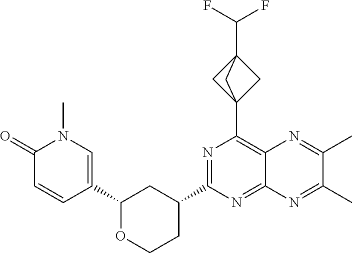 [SMILES: Cc1nc2nc([C@@H]3CCO[C@H](c4ccc(=O)n(C)c4)C3)nc(C34CC(C(F)F)(C3)C4)c2nc1C] |
| 33 | 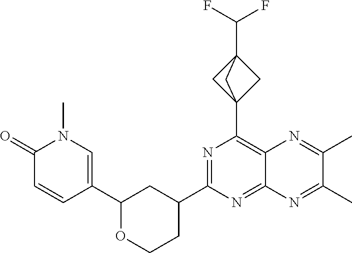 [SMILES: Cc1nc2nc(C3CCOC(c4ccc(=O)n(C)c4)C3)nc(C34CC(C(F)F)(C3)C4)c2nc1C] |
| 34 | 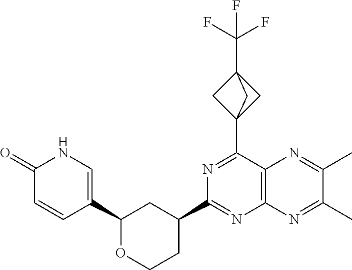 [SMILES: Cc1nc2nc([C@H]3CCO[C@@H](c4ccc(=O)[nH]c4)C3)nc(C34CC(C(F)(F)F)(C3)C4)c2nc1C] |
| 35 | 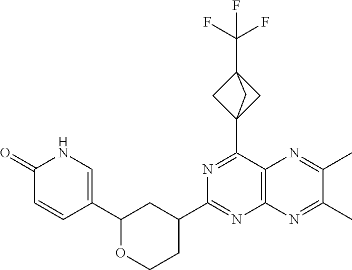 [SMILES: Cc1nc2nc(C3CCOC(c4ccc(=O)[nH]c4)C3)nc(C34CC(C(F)(F)F)(C3)C4)c2nc1C] |
| 36 | 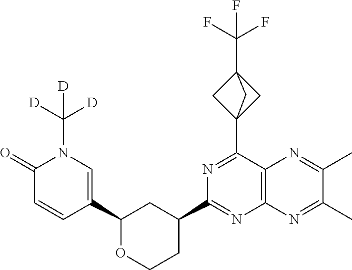 [SMILES: [2H]C([2H])([2H])n1cc([C@H]2C[C@@H](c3nc(C45CC(C(F)(F)F)(C4)C5)c4nc(C)c(C)nc4n3)CCO2)ccc1=O] |
| 37 | 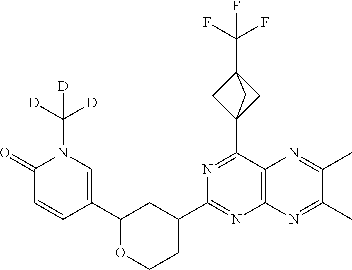 [SMILES: [2H]C([2H])([2H])n1cc(C2CC(c3nc(C45CC(C(F)(F)F)(C4)C5)c4nc(C)c(C)nc4n3)CCO2)ccc1=O] |
| 38 | 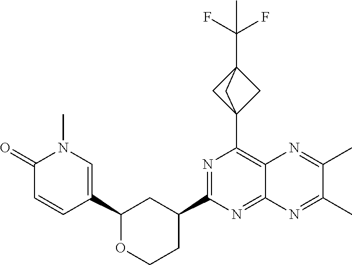 [SMILES: Cc1nc2nc([C@H]3CCO[C@@H](c4ccc(=O)n(C)c4)C3)nc(C34CC(C(C)(F)F)(C3)C4)c2nc1C] |
| 39 | 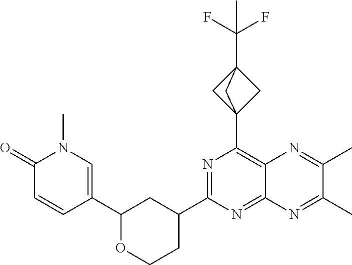 [SMILES: Cc1nc2nc(C3CCOC(c4ccc(=O)n(C)c4)C3)nc(C34CC(C(C)(F)F)(C3)C4)c2nc1C] |
| 40 | 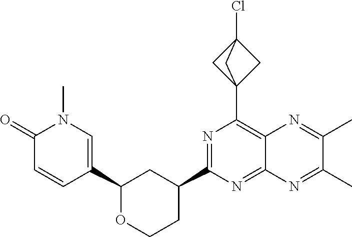 [SMILES: Cc1nc2nc([C@H]3CCO[C@@H](c4ccc(=O)n(C)c4)C3)nc(C34CC(Cl)(C3)C4)c2nc1C] |
| 41 | 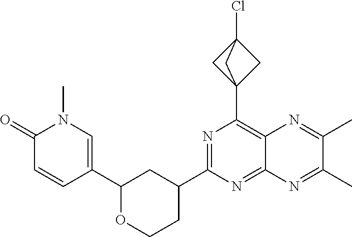 [SMILES: Cc1nc2nc(C3CCOC(c4ccc(=O)n(C)c4)C3)nc(C34CC(Cl)(C3)C4)c2nc1C] |
| 42 | 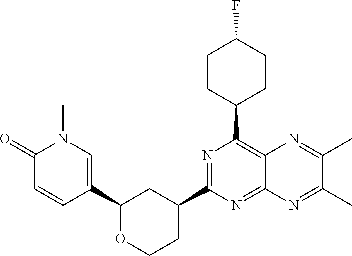 [SMILES: Cc1nc2nc([C@H]3CCO[C@@H](c4ccc(=O)n(C)c4)C3)nc([C@H]3CC[C@H](F)CC3)c2nc1C] |
| 43 | 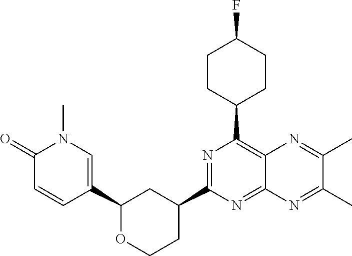 [SMILES: Cc1nc2nc([C@H]3CCO[C@@H](c4ccc(=O)n(C)c4)C3)nc([C@H]3CC[C@@H](F)CC3)c2nc1C] |
| 44 | 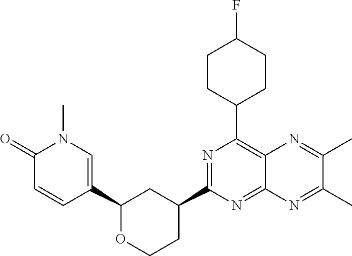 [SMILES: Cc1nc2nc([C@H]3CCO[C@@H](c4ccc(=O)n(C)c4)C3)nc(C3CCC(F)CC3)c2nc1C] |
| 45 | 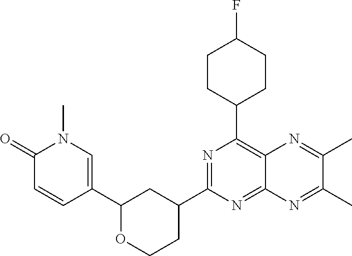 [SMILES: Cc1nc2nc(C3CCOC(c4ccc(=O)n(C)c4)C3)nc(C3CCC(F)CC3)c2nc1C] |
| 46 | 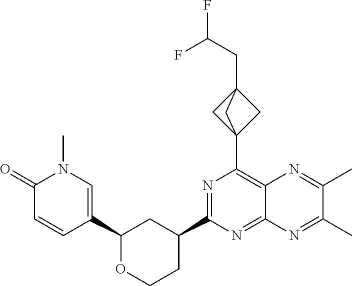 [SMILES: Cc1nc2nc([C@H]3CCO[C@@H](c4ccc(=O)n(C)c4)C3)nc(C34CC(CC(F)F)(C3)C4)c2nc1C] |
| 47 | 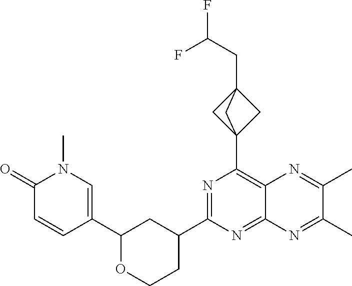 [SMILES: Cc1nc2nc(C3CCOC(c4ccc(=O)n(C)c4)C3)nc(C34CC(CC(F)F)(C3)C4)c2nc1C] |
| 48 | 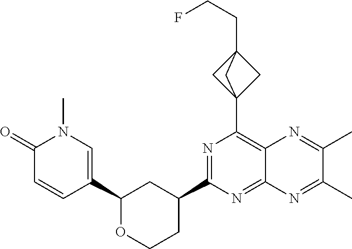 [SMILES: Cc1nc2nc([C@H]3CCO[C@@H](c4ccc(=O)n(C)c4)C3)nc(C34CC(CCF)(C3)C4)c2nc1C] |
| 49 | 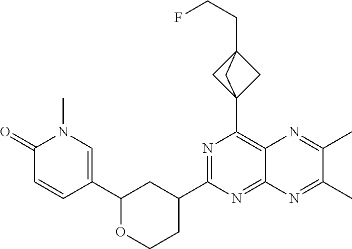 [SMILES: Cc1nc2nc(C3CCOC(c4ccc(=O)n(C)c4)C3)nc(C34CC(CCF)(C3)C4)c2nc1C] |
| 50 | 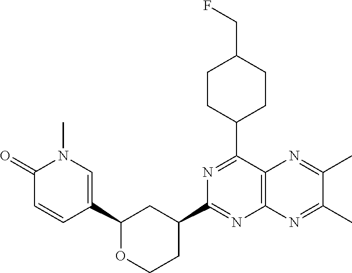 [SMILES: Cc1nc2nc([C@H]3CCO[C@@H](c4ccc(=O)n(C)c4)C3)nc(C3CCC(CF)CC3)c2nc1C] |
| 51 | 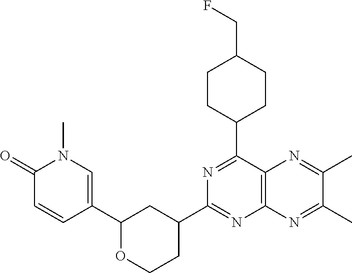 [SMILES: Cc1nc2nc(C3CCOC(c4ccc(=O)n(C)c4)C3)nc(C3CCC(CF)CC3)c2nc1C] |
| 52 | 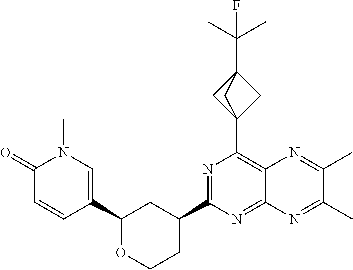 [SMILES: Cc1nc2nc([C@H]3CCO[C@@H](c4ccc(=O)n(C)c4)C3)nc(C34CC(C(C)(C)F)(C3)C4)c2nc1C] |
| 53 | 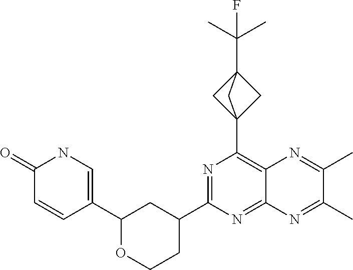 [SMILES: Cc1nc2nc(C3CCOC(c4ccc(=O)[nH]c4)C3)nc(C34CC(C(C)(C)F)(C3)C4)c2nc1C] |
| 54 | 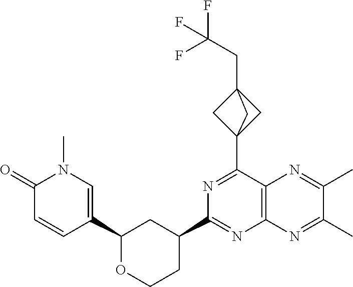 [SMILES: Cc1nc2nc([C@H]3CCO[C@@H](c4ccc(=O)n(C)c4)C3)nc(C34CC(CC(F)(F)F)(C3)C4)c2nc1C] |
| 55 | 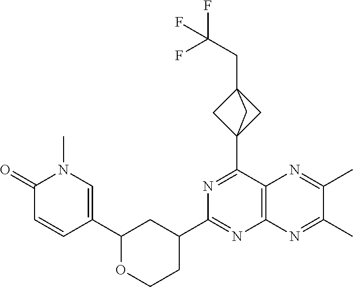 [SMILES: Cc1nc2nc(C3CCOC(c4ccc(=O)n(C)c4)C3)nc(C34CC(CC(F)(F)F)(C3)C4)c2nc1C] |
| 56 | 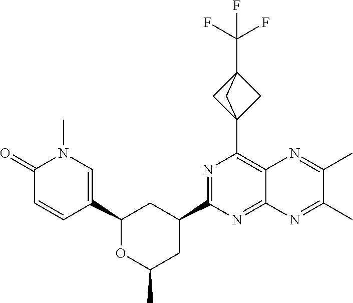 [SMILES: Cc1nc2nc([C@H]3C[C@@H](C)O[C@@H](c4ccc(=O)n(C)c4)C3)nc(C34CC(C(F)(F)F)(C3)C4)c2nc1C] |
| 57 | 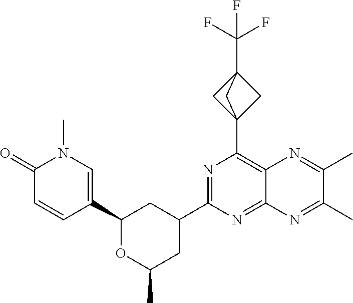 [SMILES: Cc1nc2nc(C3C[C@@H](C)O[C@@H](c4ccc(=O)n(C)c4)C3)nc(C34CC(C(F)(F)F)(C3)C4)c2nc1C] |
| 58 | 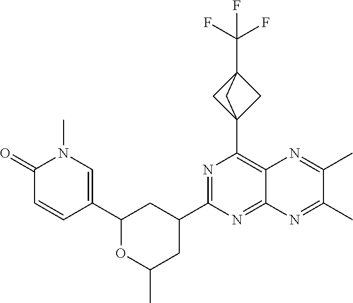 [SMILES: Cc1nc2nc(C3CC(C)OC(c4ccc(=O)n(C)c4)C3)nc(C34CC(C(F)(F)F)(C3)C4)c2nc1C] |
| 59 | 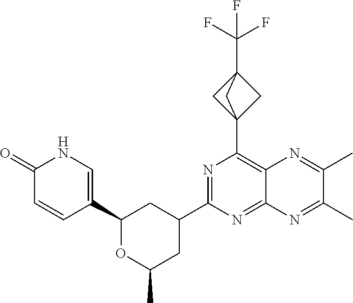 [SMILES: Cc1nc2nc(C3C[C@@H](C)O[C@@H](c4ccc(=O)[nH]c4)C3)nc(C34CC(C(F)(F)F)(C3)C4)c2nc1C] |
| 60 | 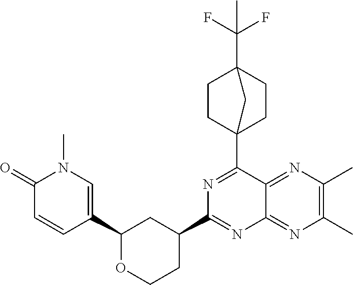 [SMILES: Cc1nc2nc([C@H]3CCO[C@@H](c4ccc(=O)n(C)c4)C3)nc(C34CCC(C(C)(F)F)(CC3)C4)c2nc1C] |
| 61 | 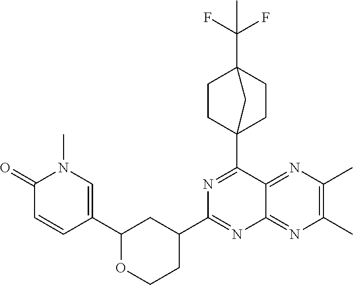 [SMILES: Cc1nc2nc(C3CCOC(c4ccc(=O)n(C)c4)C3)nc(C34CCC(C(C)(F)F)(CC3)C4)c2nc1C] |
| 62 | 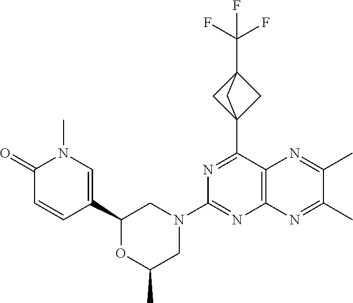 [SMILES: Cc1nc2nc(N3C[C@@H](C)O[C@@H](c4ccc(=O)n(C)c4)C3)nc(C34CC(C(F)(F)F)(C3)C4)c2nc1C] |
| 63 | 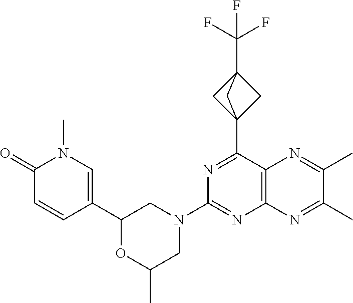 [SMILES: Cc1nc2nc(N3CC(C)OC(c4ccc(=O)n(C)c4)C3)nc(C34CC(C(F)(F)F)(C3)C4)c2nc1C] |
| 64 | 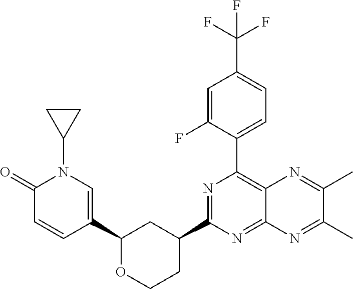 [SMILES: Cc1nc2nc([C@H]3CCO[C@@H](c4ccc(=O)n(C5CC5)c4)C3)nc(-c3ccc(C(F)(F)F)cc3F)c2nc1C] |
| 65 | 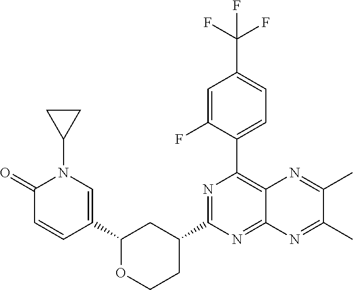 [SMILES: Cc1nc2nc([C@@H]3CCO[C@H](c4ccc(=O)n(C5CC5)c4)C3)nc(-c3ccc(C(F)(F)F)cc3F)c2nc1C] |
| 66 | 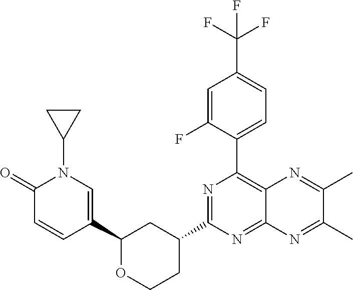 [SMILES: Cc1nc2nc([C@@H]3CCO[C@@H](c4ccc(=O)n(C5CC5)c4)C3)nc(-c3ccc(C(F)(F)F)cc3F)c2nc1C] |
| 67 | 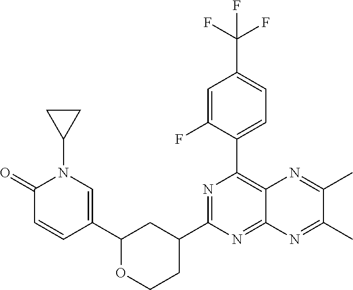 [SMILES: Cc1nc2nc(C3CCOC(c4ccc(=O)n(C5CC5)c4)C3)nc(-c3ccc(C(F)(F)F)cc3F)c2nc1C] |
| 68 | 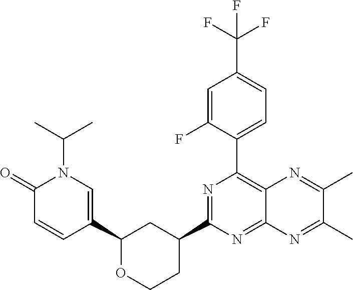 [SMILES: Cc1nc2nc([C@H]3CCO[C@@H](c4ccc(=O)n(C(C)C)c4)C3)nc(-c3ccc(C(F)(F)F)cc3F)c2nc1C] |
| 69 | 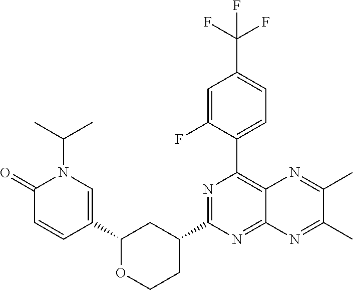 [SMILES: Cc1nc2nc([C@@H]3CCO[C@H](c4ccc(=O)n(C(C)C)c4)C3)nc(-c3ccc(C(F)(F)F)cc3F)c2nc1C] |
| 70 | 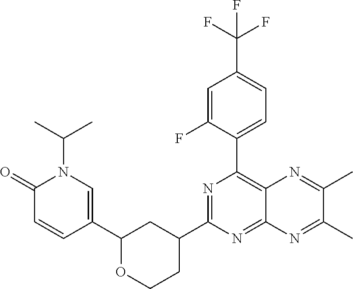 [SMILES: Cc1nc2nc(C3CCOC(c4ccc(=O)n(C(C)C)c4)C3)nc(-c3ccc(C(F)(F)F)cc3F)c2nc1C] |
| 71 | 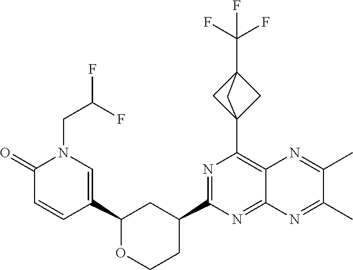 [SMILES: Cc1nc2nc([C@H]3CCO[C@@H](c4ccc(=O)n(CC(F)F)c4)C3)nc(C34CC(C(F)(F)F)(C3)C4)c2nc1C] |
| 72 | 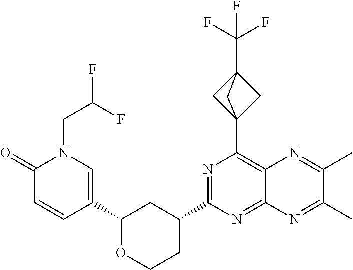 [SMILES: Cc1nc2nc([C@@H]3CCO[C@H](c4ccc(=O)n(CC(F)F)c4)C3)nc(C34CC(C(F)(F)F)(C3)C4)c2nc1C] |
| 73 | 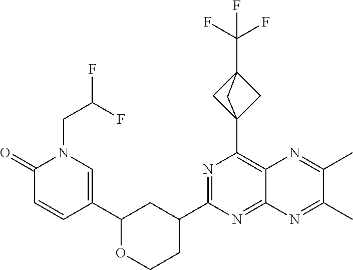 [SMILES: Cc1nc2nc(C3CCOC(c4ccc(=O)n(CC(F)F)c4)C3)nc(C34CC(C(F)(F)F)(C3)C4)c2nc1C] |
| 74 | 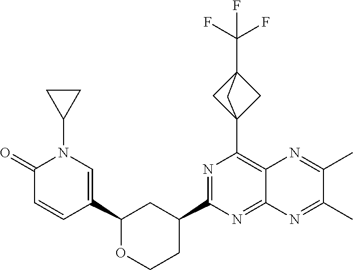 [SMILES: Cc1nc2nc([C@H]3CCO[C@@H](c4ccc(=O)n(C5CC5)c4)C3)nc(C34CC(C(F)(F)F)(C3)C4)c2nc1C] |
| 75 | 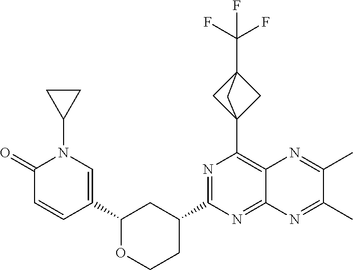 [SMILES: Cc1nc2nc([C@@H]3CCO[C@H](c4ccc(=O)n(C5CC5)c4)C3)nc(C34CC(C(F)(F)F)(C3)C4)c2nc1C] |
| 76 | 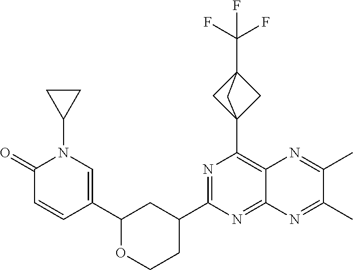 [SMILES: Cc1nc2nc(C3CCOC(c4ccc(=O)n(C5CC5)c4)C3)nc(C34CC(C(F)(F)F)(C3)C4)c2nc1C] |
| 77 | 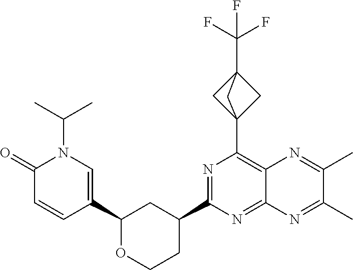 [SMILES: Cc1nc2nc([C@H]3CCO[C@@H](c4ccc(=O)n(C(C)C)c4)C3)nc(C34CC(C(F)(F)F)(C3)C4)c2nc1C] |
| 78 | 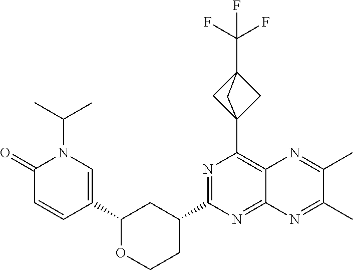 [SMILES: Cc1nc2nc([C@@H]3CCO[C@H](c4ccc(=O)n(C(C)C)c4)C3)nc(C34CC(C(F)(F)F)(C3)C4)c2nc1C] |
| 79 | 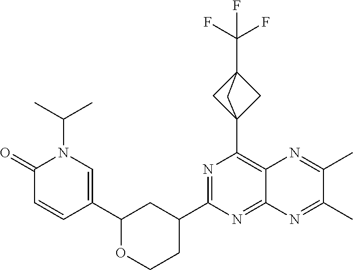 [SMILES: Cc1nc2nc(C3CCOC(c4ccc(=O)n(C(C)C)c4)C3)nc(C34CC(C(F)(F)F)(C3)C4)c2nc1C] |
| 80 | 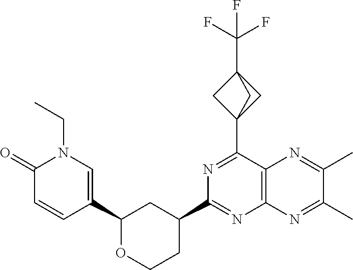 [SMILES: CCn1cc([C@H]2C[C@@H](c3nc(C45CC(C(F)(F)F)(C4)C5)c4nc(C)c(C)nc4n3)CCO2)ccc1=O] |
| 81 | 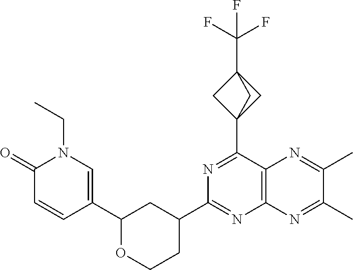 [SMILES: CCn1cc(C2CC(c3nc(C45CC(C(F)(F)F)(C4)C5)c4nc(C)c(C)nc4n3)CCO2)ccc1=O] |
| 82 | 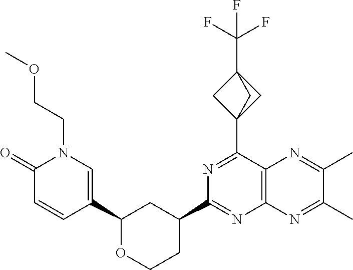 [SMILES: COCCn1cc([C@H]2C[C@@H](c3nc(C45CC(C(F)(F)F)(C4)C5)c4nc(C)c(C)nc4n3)CCO2)ccc1=O] |
| 83 | 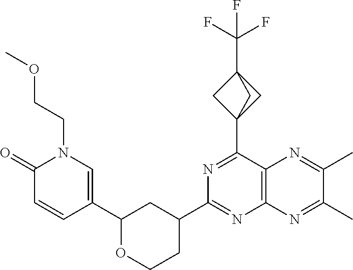 [SMILES: COCCn1cc(C2CC(c3nc(C45CC(C(F)(F)F)(C4)C5)c4nc(C)c(C)nc4n3)CCO2)ccc1=O] |
| 84 | 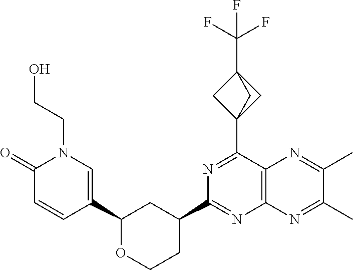 [SMILES: Cc1nc2nc([C@H]3CCO[C@@H](c4ccc(=O)n(CCO)c4)C3)nc(C34CC(C(F)(F)F)(C3)C4)c2nc1C] |
| 85 | 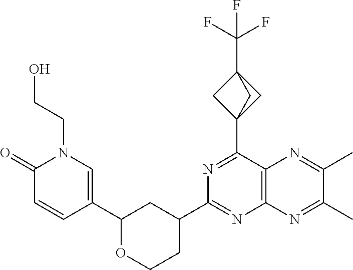 [SMILES: Cc1nc2nc(C3CCOC(c4ccc(=O)n(CCO)c4)C3)nc(C34CC(C(F)(F)F)(C3)C4)c2nc1C] |
| 86 | 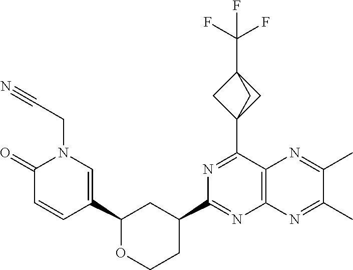 [SMILES: Cc1nc2nc([C@H]3CCO[C@@H](c4ccc(=O)n(CC#N)c4)C3)nc(C34CC(C(F)(F)F)(C3)C4)c2nc1C] |
| 87 | 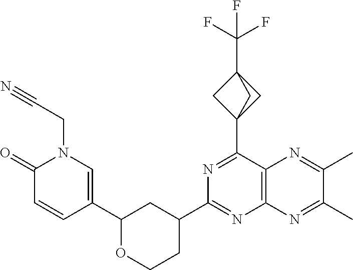 [SMILES: Cc1nc2nc(C3CCOC(c4ccc(=O)n(CC#N)c4)C3)nc(C34CC(C(F)(F)F)(C3)C4)c2nc1C] |
| 88 | 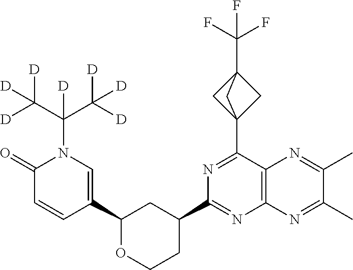 [SMILES: [2H]C([2H])([2H])C([2H])(n1cc([C@H]2C[C@@H](c3nc(C45CC(C(F)(F)F)(C4)C5)c4nc(C)c(C)nc4n3)CCO2)ccc1=O)C([2H])([2H])[2H]] |
| 89 | 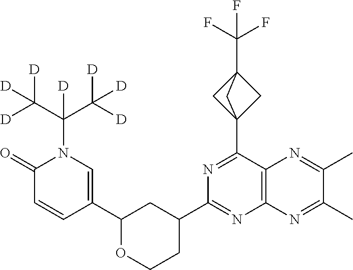 [SMILES: [2H]C([2H])([2H])C([2H])(n1cc(C2CC(c3nc(C45CC(C(F)(F)F)(C4)C5)c4nc(C)c(C)nc4n3)CCO2)ccc1=O)C([2H])([2H])[2H]] |
| 90 | 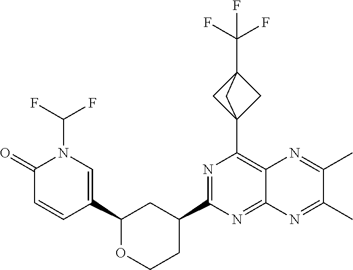 [SMILES: Cc1nc2nc([C@H]3CCO[C@@H](c4ccc(=O)n(C(F)F)c4)C3)nc(C34CC(C(F)(F)F)(C3)C4)c2nc1C] |
| 91 | 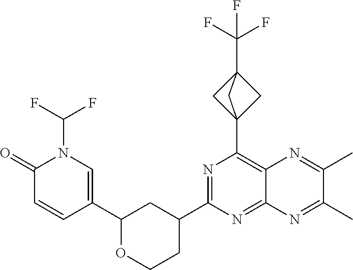 [SMILES: Cc1nc2nc(C3CCOC(c4ccc(=O)n(C(F)F)c4)C3)nc(C34CC(C(F)(F)F)(C3)C4)c2nc1C] |
| 92 | 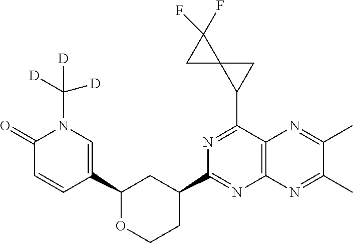 [SMILES: [2H]C([2H])([2H])n1cc([C@H]2C[C@@H](c3nc(C4CC45CC5(F)F)c4nc(C)c(C)nc4n3)CCO2)ccc1=O] |
| 93 | 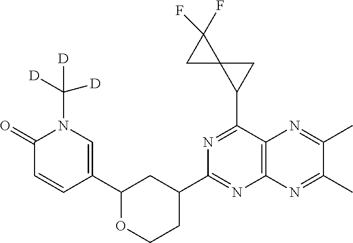 [SMILES: [2H]C([2H])([2H])n1cc(C2CC(c3nc(C4CC45CC5(F)F)c4nc(C)c(C)nc4n3)CCO2)ccc1=O] |
| 94 | 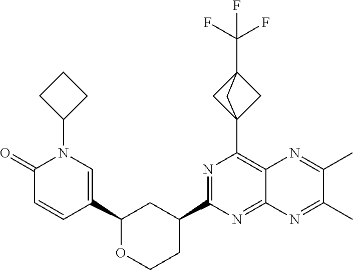 [SMILES: Cc1nc2nc([C@H]3CCO[C@@H](c4ccc(=O)n(C5CCC5)c4)C3)nc(C34CC(C(F)(F)F)(C3)C4)c2nc1C] |
| 95 | 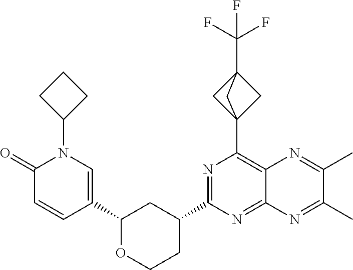 [SMILES: Cc1nc2nc([C@@H]3CCO[C@H](c4ccc(=O)n(C5CCC5)c4)C3)nc(C34CC(C(F)(F)F)(C3)C4)c2nc1C] |
| 96 | 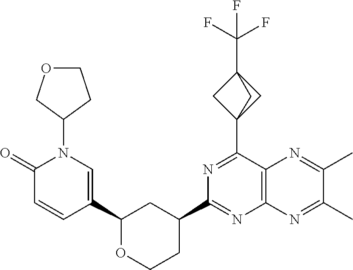 [SMILES: Cc1nc2nc([C@H]3CCO[C@@H](c4ccc(=O)n(C5CCOC5)c4)C3)nc(C34CC(C(F)(F)F)(C3)C4)c2nc1C] |
| 97 | 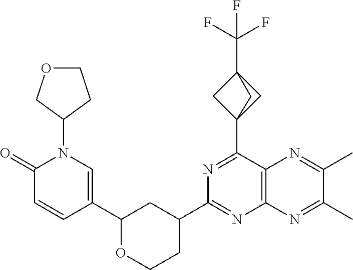 [SMILES: Cc1nc2nc(C3CCOC(c4ccc(=O)n(C5CCOC5)c4)C3)nc(C34CC(C(F)(F)F)(C3)C4)c2nc1C] |
| 98 | 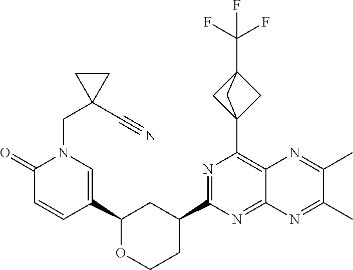 [SMILES: Cc1nc2nc([C@H]3CCO[C@@H](c4ccc(=O)n(CC5(C#N)CC5)c4)C3)nc(C34CC(C(F)(F)F)(C3)C4)c2nc1C] |
| 99 | 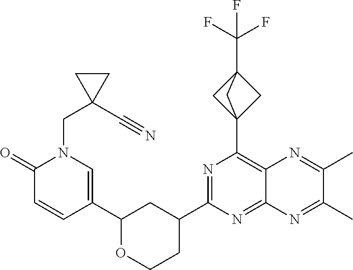 [SMILES: Cc1nc2nc(C3CCOC(c4ccc(=O)n(CC5(C#N)CC5)c4)C3)nc(C34CC(C(F)(F)F)(C3)C4)c2nc1C] |
| 100 | 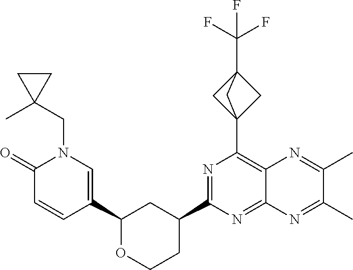 [SMILES: Cc1nc2nc([C@H]3CCO[C@@H](c4ccc(=O)n(CC5(C)CC5)c4)C3)nc(C34CC(C(F)(F)F)(C3)C4)c2nc1C] |
| 101 | 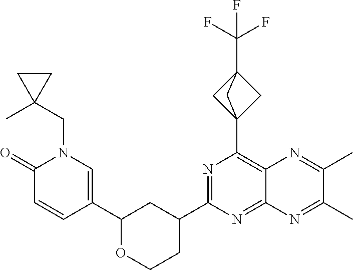 [SMILES: Cc1nc2nc(C3CCOC(c4ccc(=O)n(CC5(C)CC5)c4)C3)nc(C34CC(C(F)(F)F)(C3)C4)c2nc1C] |
| 102 | 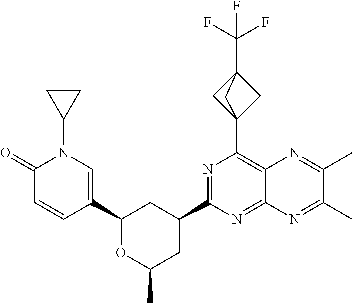 [SMILES: Cc1nc2nc([C@H]3C[C@@H](C)O[C@@H](c4ccc(=O)n(C5CC5)c4)C3)nc(C34CC(C(F)(F)F)(C3)C4)c2nc1C] |
| 103 | 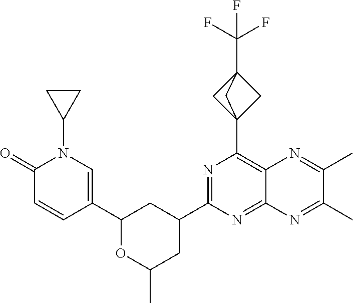 [SMILES: Cc1nc2nc(C3CC(C)OC(c4ccc(=O)n(C5CC5)c4)C3)nc(C34CC(C(F)(F)F)(C3)C4)c2nc1C] |
| 104 | 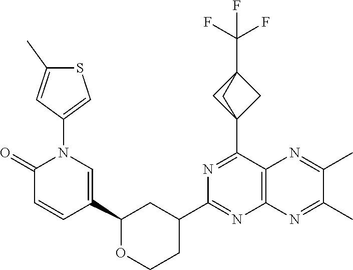 [SMILES: Cc1cc(-n2cc([C@H]3CC(c4nc(C56CC(C(F)(F)F)(C5)C6)c5nc(C)c(C)nc5n4)CCO3)ccc2=O)cs1] |
| 105 | 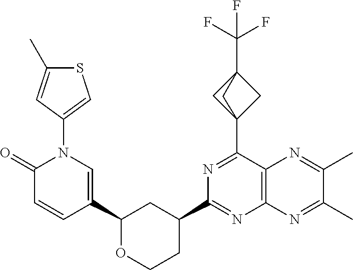 [SMILES: Cc1cc(-n2cc([C@H]3C[C@@H](c4nc(C56CC(C(F)(F)F)(C5)C6)c5nc(C)c(C)nc5n4)CCO3)ccc2=O)cs1] |
| 106 | 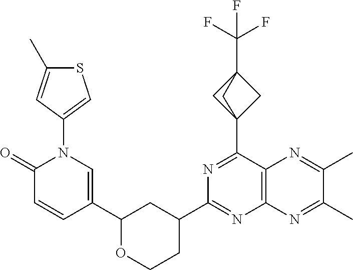 [SMILES: Cc1cc(-n2cc(C3CC(c4nc(C56CC(C(F)(F)F)(C5)C6)c5nc(C)c(C)nc5n4)CCO3)ccc2=O)cs1] |
| 107 | 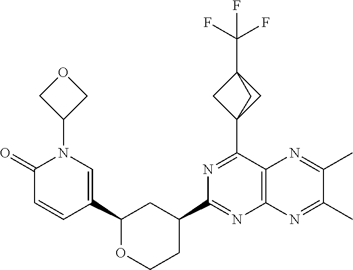 [SMILES: Cc1nc2nc([C@H]3CCO[C@@H](c4ccc(=O)n(C5COC5)c4)C3)nc(C34CC(C(F)(F)F)(C3)C4)c2nc1C] |
| 108 | 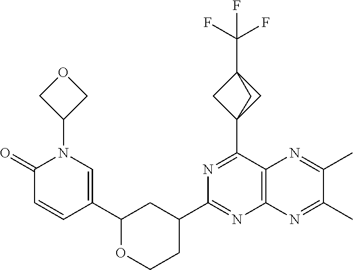 [SMILES: Cc1nc2nc(C3CCOC(c4ccc(=O)n(C5COC5)c4)C3)nc(C34CC(C(F)(F)F)(C3)C4)c2nc1C] |
| 109 | 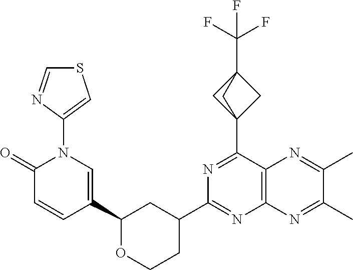 [SMILES: Cc1nc2nc(C3CCO[C@@H](c4ccc(=O)n(-c5cscn5)c4)C3)nc(C34CC(C(F)(F)F)(C3)C4)c2nc1C] |
| 110 | 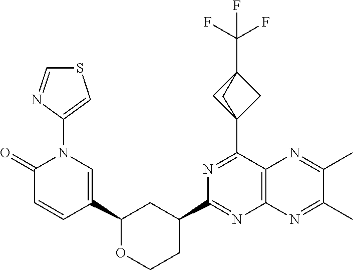 [SMILES: Cc1nc2nc([C@H]3CCO[C@@H](c4ccc(=O)n(-c5cscn5)c4)C3)nc(C34CC(C(F)(F)F)(C3)C4)c2nc1C] |
| 111 | 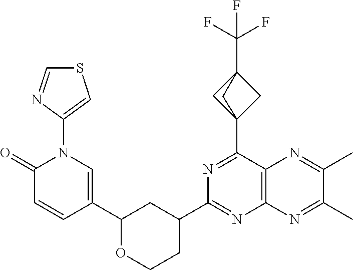 [SMILES: Cc1nc2nc(C3CCOC(c4ccc(=O)n(-c5cscn5)c4)C3)nc(C34CC(C(F)(F)F)(C3)C4)c2nc1C] |
| 112 | 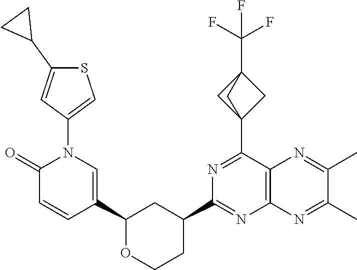 [SMILES: Cc1nc2nc([C@H]3CCO[C@@H](c4ccc(=O)n(-c5csc(C6CC6)c5)c4)C3)nc(C34CC(C(F)(F)F)(C3)C4)c2nc1C] |
| 113 | 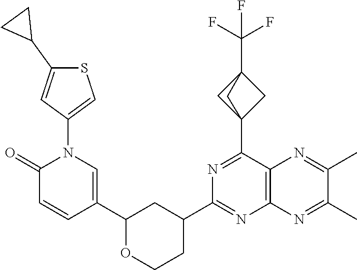 [SMILES: Cc1nc2nc(C3CCOC(c4ccc(=O)n(-c5csc(C6CC6)c5)c4)C3)nc(C34CC(C(F)(F)F)(C3)C4)c2nc1C] |
| 114 | 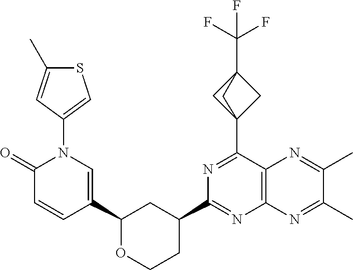 [SMILES: Cc1cc(-n2cc([C@H]3C[C@@H](c4nc(C56CC(C(F)(F)F)(C5)C6)c5nc(C)c(C)nc5n4)CCO3)ccc2=O)cs1] |
| 115 | 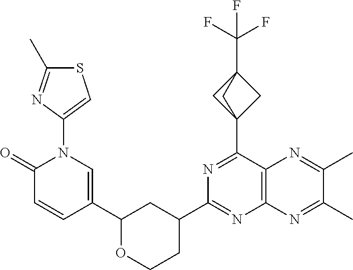 [SMILES: Cc1nc(-n2cc(C3CC(c4nc(C56CC(C(F)(F)F)(C5)C6)c5nc(C)c(C)nc5n4)CCO3)ccc2=O)cs1] |
| 116 | 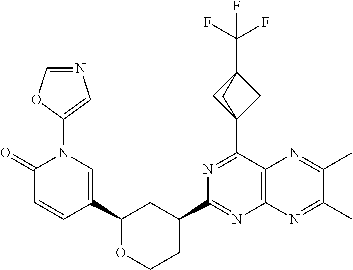 [SMILES: Cc1nc2nc([C@H]3CCO[C@@H](c4ccc(=O)n(-c5cnco5)c4)C3)nc(C34CC(C(F)(F)F)(C3)C4)c2nc1C] |
| 117 | 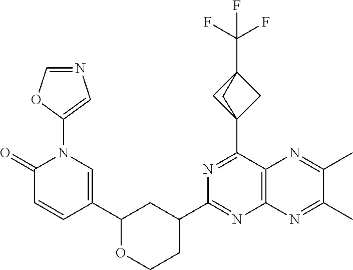 [SMILES: Cc1nc2nc(C3CCOC(c4ccc(=O)n(-c5cnco5)c4)C3)nc(C34CC(C(F)(F)F)(C3)C4)c2nc1C] |
| 118 | 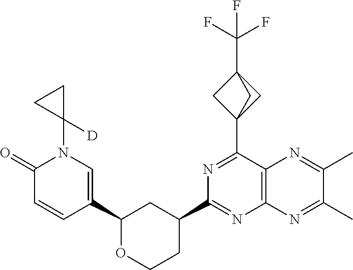 [SMILES: [2H]C1(n2cc([C@H]3C[C@@H](c4nc(C56CC(C(F)(F)F)(C5)C6)c5nc(C)c(C)nc5n4)CCO3)ccc2=O)CC1] |
| 119 | 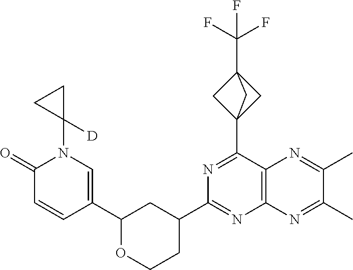 [SMILES: [2H]C1(n2cc(C3CC(c4nc(C56CC(C(F)(F)F)(C5)C6)c5nc(C)c(C)nc5n4)CCO3)ccc2=O)CC1] |
| 120 | 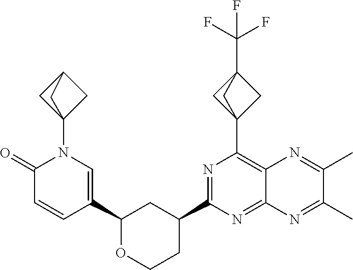 [SMILES: Cc1nc2nc([C@H]3CCO[C@@H](c4ccc(=O)n(C56CC(C5)C6)c4)C3)nc(C34CC(C(F)(F)F)(C3)C4)c2nc1C] |
| 121 | 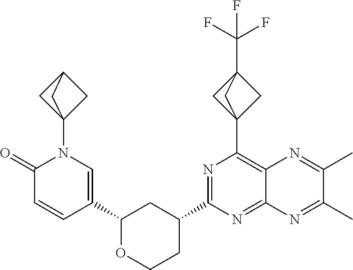 [SMILES: Cc1nc2nc([C@@H]3CCO[C@H](c4ccc(=O)n(C56CC(C5)C6)c4)C3)nc(C34CC(C(F)(F)F)(C3)C4)c2nc1C] |
| 122 | 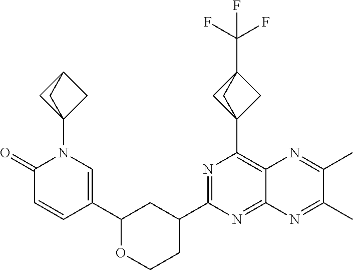 [SMILES: Cc1nc2nc(C3CCOC(c4ccc(=O)n(C56CC(C5)C6)c4)C3)nc(C34CC(C(F)(F)F)(C3)C4)c2nc1C] |
| 123 | 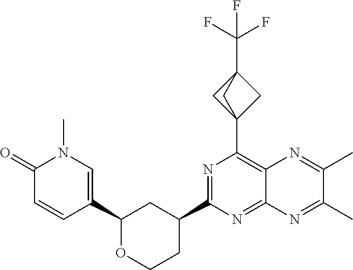 [SMILES: Cc1nc2nc([C@H]3CCO[C@@H](c4ccc(=O)n(C)c4)C3)nc(C34CC(C(F)(F)F)(C3)C4)c2nc1C] |
| 124 | 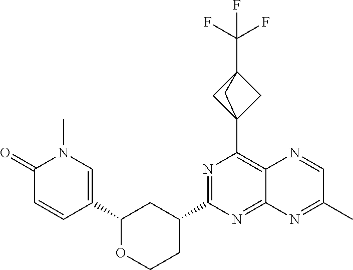 [SMILES: Cc1cnc2c(C34CC(C(F)(F)F)(C3)C4)nc([C@@H]3CCO[C@H](c4ccc(=O)n(C)c4)C3)nc2n1] |
| 125 | 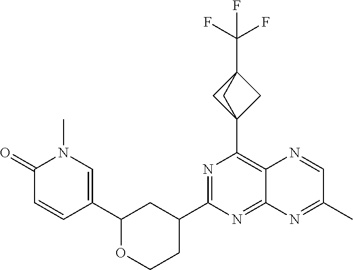 [SMILES: Cc1cnc2c(C34CC(C(F)(F)F)(C3)C4)nc(C3CCOC(c4ccc(=O)n(C)c4)C3)nc2n1] |
| 126 | 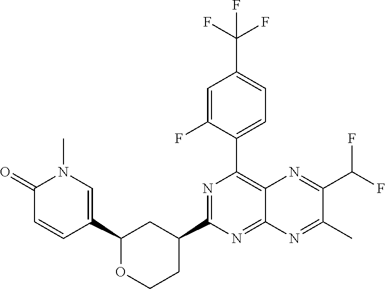 [SMILES: Cc1nc2nc([C@H]3CCO[C@@H](c4ccc(=O)n(C)c4)C3)nc(-c3ccc(C(F)(F)F)cc3F)c2nc1C(F)F] |
| 127 | 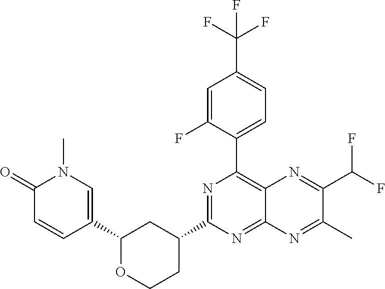 [SMILES: Cc1nc2nc([C@@H]3CCO[C@H](c4ccc(=O)n(C)c4)C3)nc(-c3ccc(C(F)(F)F)cc3F)c2nc1C(F)F] |
| 128 | 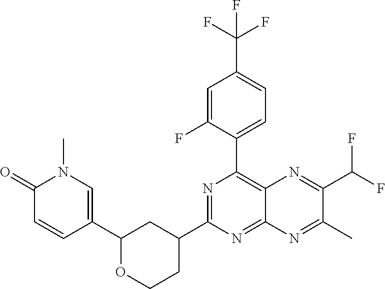 [SMILES: Cc1nc2nc(C3CCOC(c4ccc(=O)n(C)c4)C3)nc(-c3ccc(C(F)(F)F)cc3F)c2nc1C(F)F] |
| 129 | 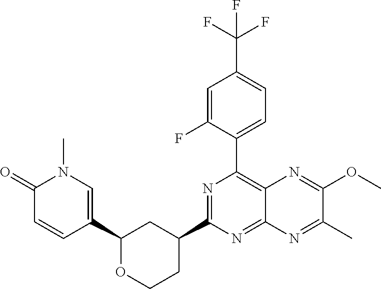 [SMILES: COc1nc2c(-c3ccc(C(F)(F)F)cc3F)nc([C@H]3CCO[C@@H](c4ccc(=O)n(C)c4)C3)nc2nc1C] |
| 130 | 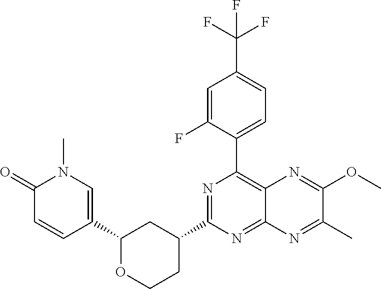 [SMILES: COc1nc2c(-c3ccc(C(F)(F)F)cc3F)nc([C@@H]3CCO[C@H](c4ccc(=O)n(C)c4)C3)nc2nc1C] |
| 131 | 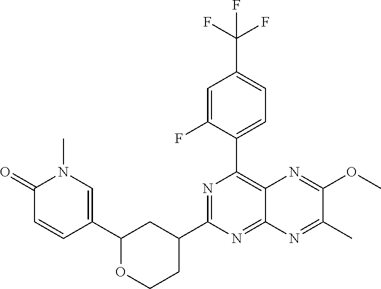 [SMILES: COc1nc2c(-c3ccc(C(F)(F)F)cc3F)nc(C3CCOC(c4ccc(=O)n(C)c4)C3)nc2nc1C] |
| 132 | 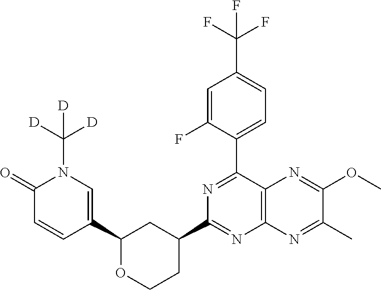 [SMILES: [2H]C([2H])([2H])n1cc([C@H]2C[C@@H](c3nc(-c4ccc(C(F)(F)F)cc4F)c4nc(OC)c(C)nc4n3)CCO2)ccc1=O] |
| 133 | 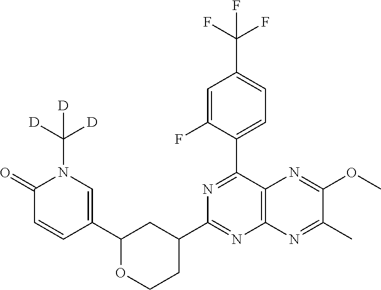 [SMILES: [2H]C([2H])([2H])n1cc(C2CC(c3nc(-c4ccc(C(F)(F)F)cc4F)c4nc(OC)c(C)nc4n3)CCO2)ccc1=O] |
| 134 | 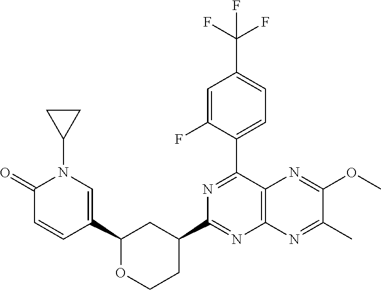 [SMILES: COc1nc2c(-c3ccc(C(F)(F)F)cc3F)nc([C@H]3CCO[C@@H](c4ccc(=O)n(C5CC5)c4)C3)nc2nc1C] |
| 135 | 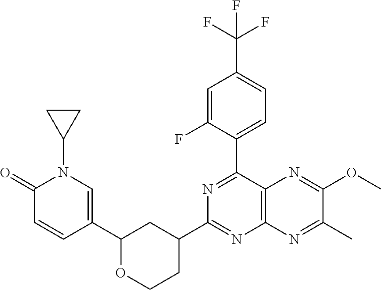 [SMILES: COc1nc2c(-c3ccc(C(F)(F)F)cc3F)nc(C3CCOC(c4ccc(=O)n(C5CC5)c4)C3)nc2nc1C] |
| 136 | 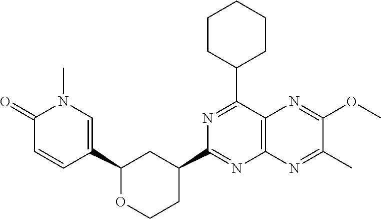 [SMILES: COc1nc2c(C3CCCCC3)nc([C@H]3CCO[C@@H](c4ccc(=O)n(C)c4)C3)nc2nc1C] |
| 137 | 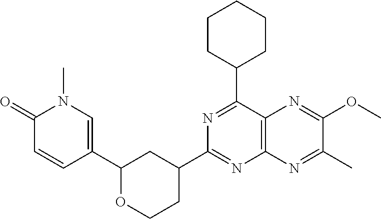 [SMILES: COc1nc2c(C3CCCCC3)nc(C3CCOC(c4ccc(=O)n(C)c4)C3)nc2nc1C] |
| 138 | 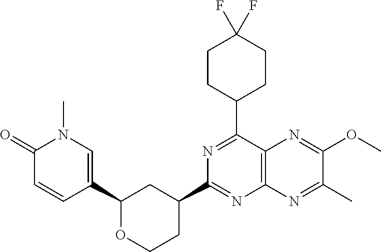 [SMILES: COc1nc2c(C3CCC(F)(F)CC3)nc([C@H]3CCO[C@@H](c4ccc(=O)n(C)c4)C3)nc2nc1C] |
| 139 | 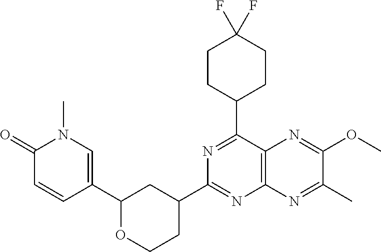 [SMILES: COc1nc2c(C3CCC(F)(F)CC3)nc(C3CCOC(c4ccc(=O)n(C)c4)C3)nc2nc1C] |
| 140 | 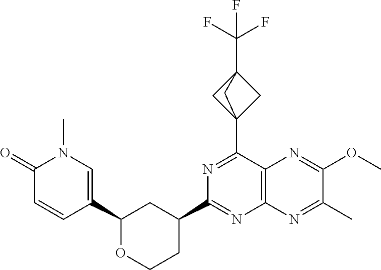 [SMILES: COc1nc2c(C34CC(C(F)(F)F)(C3)C4)nc([C@H]3CCO[C@@H](c4ccc(=O)n(C)c4)C3)nc2nc1C] |
| 141 | 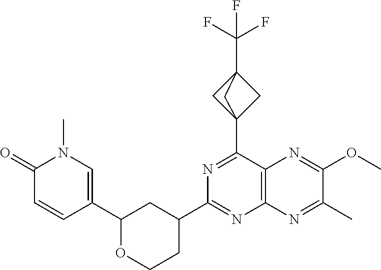 [SMILES: COc1nc2c(C34CC(C(F)(F)F)(C3)C4)nc(C3CCOC(c4ccc(=O)n(C)c4)C3)nc2nc1C] |
| 142 | 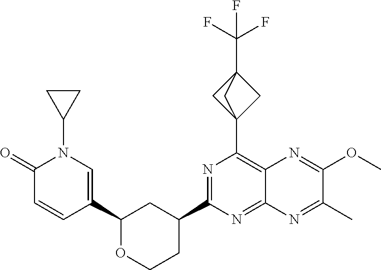 [SMILES: COc1nc2c(C34CC(C(F)(F)F)(C3)C4)nc([C@H]3CCO[C@@H](c4ccc(=O)n(C5CC5)c4)C3)nc2nc1C] |
| 143 | 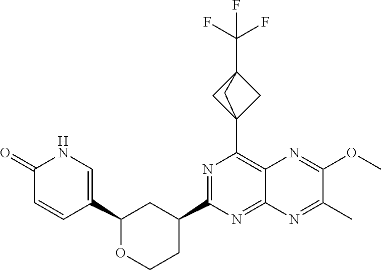 [SMILES: COc1nc2c(C34CC(C(F)(F)F)(C3)C4)nc([C@H]3CCO[C@@H](c4ccc(=O)[nH]c4)C3)nc2nc1C] |
| 144 | 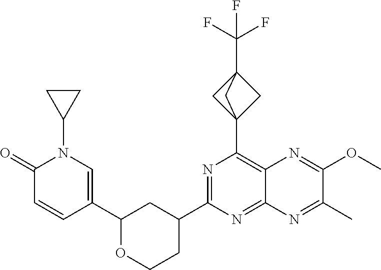 [SMILES: COc1nc2c(C34CC(C(F)(F)F)(C3)C4)nc(C3CCOC(c4ccc(=O)n(C5CC5)c4)C3)nc2nc1C] |
| 145 | 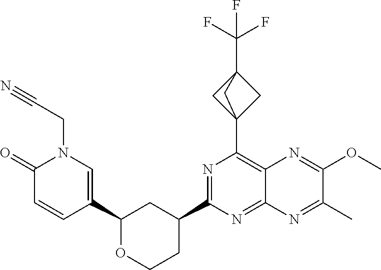 [SMILES: COc1nc2c(C34CC(C(F)(F)F)(C3)C4)nc([C@H]3CCO[C@@H](c4ccc(=O)n(CC#N)c4)C3)nc2nc1C] |
| 146 | 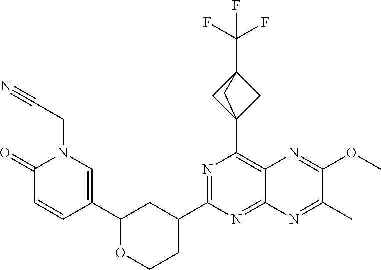 [SMILES: COc1nc2c(C34CC(C(F)(F)F)(C3)C4)nc(C3CCOC(c4ccc(=O)n(CC#N)c4)C3)nc2nc1C] |
| 147 | 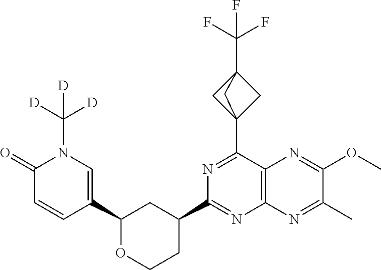 [SMILES: [2H]C([2H])([2H])n1cc([C@H]2C[C@@H](c3nc(C45CC(C(F)(F)F)(C4)C5)c4nc(OC)c(C)nc4n3)CCO2)ccc1=O] |
| 148 | 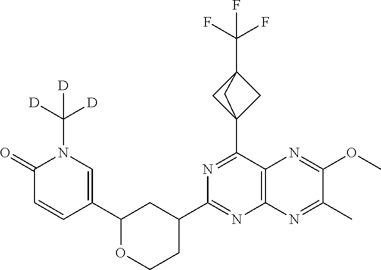 [SMILES: [2H]C([2H])([2H])n1cc(C2CC(c3nc(C45CC(C(F)(F)F)(C4)C5)c4nc(OC)c(C)nc4n3)CCO2)ccc1=O] |
| 149 | 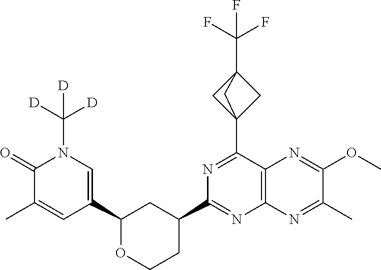 [SMILES: [2H]C([2H])([2H])n1cc([C@H]2C[C@@H](c3nc(C45CC(C(F)(F)F)(C4)C5)c4nc(OC)c(C)nc4n3)CCO2)cc(C)c1=O] |
| 150 | 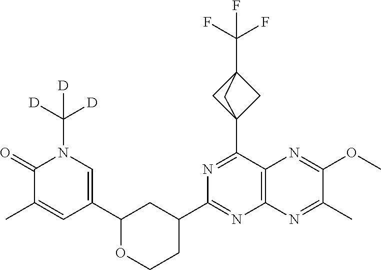 |
| 151 | 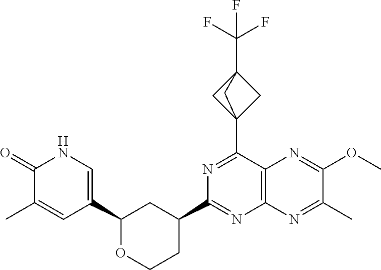 [SMILES: COc1nc2c(C34CC(C(F)(F)F)(C3)C4)nc([C@H]3CCO[C@@H](c4c[nH]c(=O)c(C)c4)C3)nc2nc1C] |
| 152 | 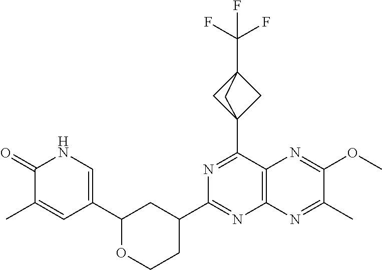 |
| 153 | 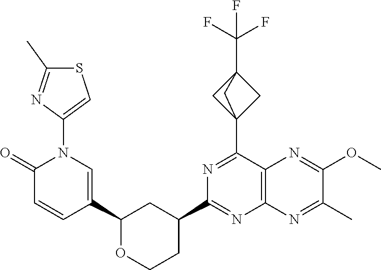 [SMILES: COc1nc2c(C34CC(C(F)(F)F)(C3)C4)nc([C@H]3CCO[C@@H](c4ccc(=O)n(-c5csc(C)n5)c4)C3)nc2nc1C] |
| 154 | 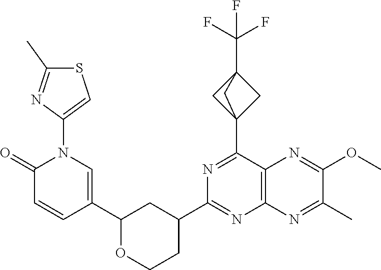 [SMILES: COc1nc2c(C34CC(C(F)(F)F)(C3)C4)nc(C3CCOC(c4ccc(=O)n(-c5csc(C)n5)c4)C3)nc2nc1C] |
| 155 | 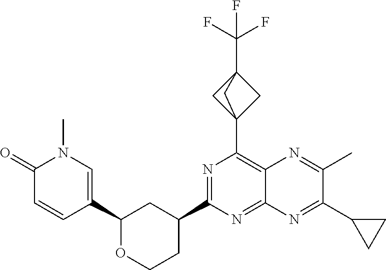 [SMILES: Cc1nc2c(C34CC(C(F)(F)F)(C3)C4)nc([C@H]3CCO[C@@H](c4ccc(=O)n(C)c4)C3)nc2nc1C1CC1] |
| 156 | 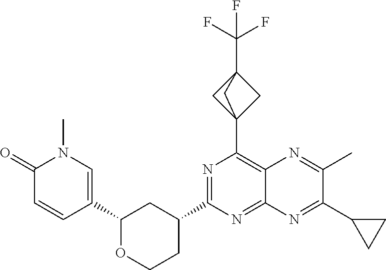 [SMILES: Cc1nc2c(C34CC(C(F)(F)F)(C3)C4)nc([C@@H]3CCO[C@H](c4ccc(=O)n(C)c4)C3)nc2nc1C1CC1] |
| 157 | 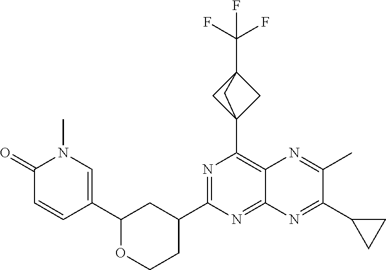 [SMILES: Cc1nc2c(C34CC(C(F)(F)F)(C3)C4)nc(C3CCOC(c4ccc(=O)n(C)c4)C3)nc2nc1C1CC1] |
| 158 | 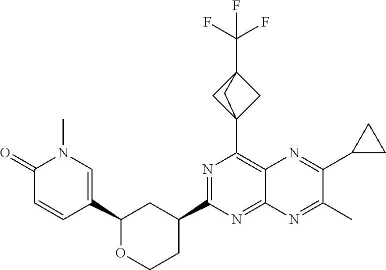 [SMILES: Cc1nc2nc([C@H]3CCO[C@@H](c4ccc(=O)n(C)c4)C3)nc(C34CC(C(F)(F)F)(C3)C4)c2nc1C1CC1] |
| 159 | 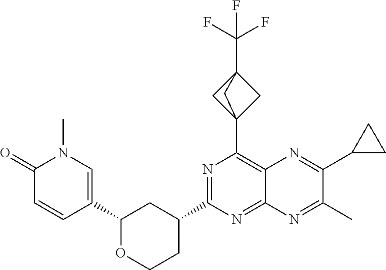 [SMILES: Cc1nc2nc([C@@H]3CCO[C@H](c4ccc(=O)n(C)c4)C3)nc(C34CC(C(F)(F)F)(C3)C4)c2nc1C1CC1] |
| 160 | 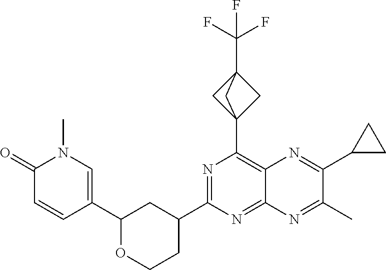 [SMILES: Cc1nc2nc(C3CCOC(c4ccc(=O)n(C)c4)C3)nc(C34CC(C(F)(F)F)(C3)C4)c2nc1C1CC1] |
| 161 | 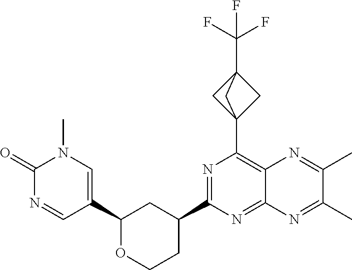 [SMILES: Cc1nc2nc([C@H]3CCO[C@@H](c4cnc(=O)n(C)c4)C3)nc(C34CC(C(F)(F)F)(C3)C4)c2nc1C] |
| 162 | 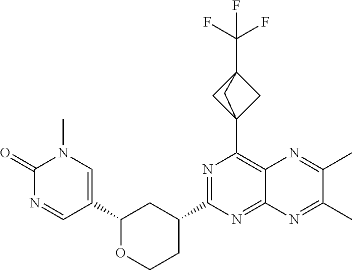 [SMILES: Cc1nc2nc([C@@H]3CCO[C@H](c4cnc(=O)n(C)c4)C3)nc(C34CC(C(F)(F)F)(C3)C4)c2nc1C] |
| 163 | 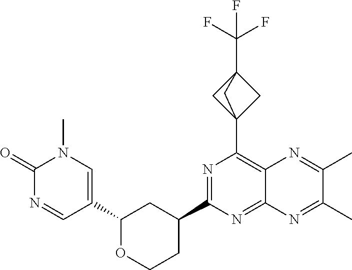 [SMILES: Cc1nc2nc([C@H]3CCO[C@H](c4cnc(=O)n(C)c4)C3)nc(C34CC(C(F)(F)F)(C3)C4)c2nc1C] |
| 164 | 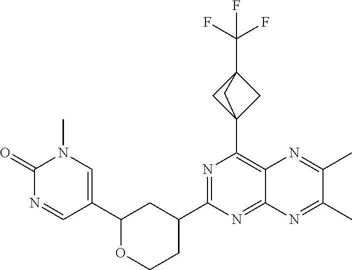 [SMILES: Cc1nc2nc(C3CCOC(c4cnc(=O)n(C)c4)C3)nc(C34CC(C(F)(F)F)(C3)C4)c2nc1C] |
| 165 | 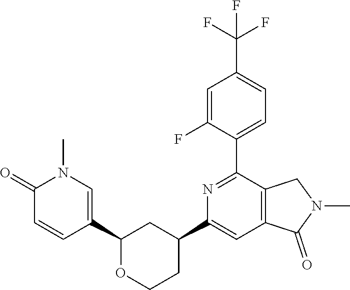 [SMILES: CN1Cc2c(cc([C@H]3CCO[C@@H](c4ccc(=O)n(C)c4)C3)nc2-c2ccc(C(F)(F)F)cc2F)C1=O] |
| 166 | 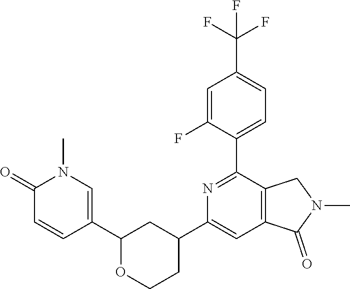 [SMILES: CN1Cc2c(cc(C3CCOC(c4ccc(=O)n(C)c4)C3)nc2-c2ccc(C(F)(F)F)cc2F)C1=O] |
| 167 | 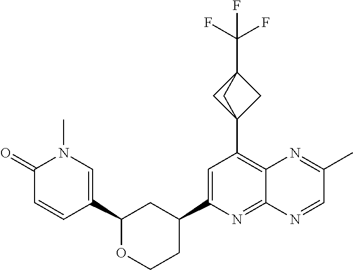 [SMILES: Cc1cnc2nc([C@H]3CCO[C@@H](c4ccc(=O)n(C)c4)C3)cc(C34CC(C(F)(F)F)(C3)C4)c2n1] |
| 168 | 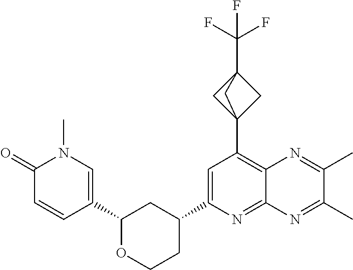 [SMILES: Cc1nc2nc([C@@H]3CCO[C@H](c4ccc(=O)n(C)c4)C3)cc(C34CC(C(F)(F)F)(C3)C4)c2nc1C] |
| 169 | 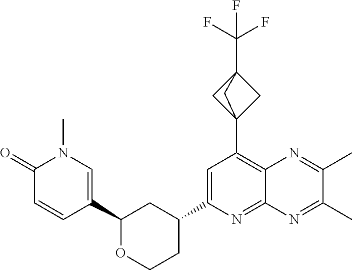 [SMILES: Cc1nc2nc([C@@H]3CCO[C@@H](c4ccc(=O)n(C)c4)C3)cc(C34CC(C(F)(F)F)(C3)C4)c2nc1C] |
| 170 | 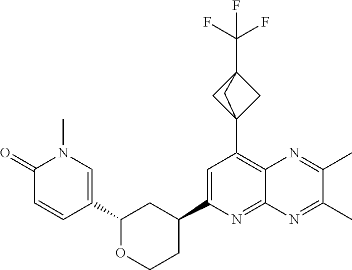 [SMILES: Cc1nc2nc([C@H]3CCO[C@H](c4ccc(=O)n(C)c4)C3)cc(C34CC(C(F)(F)F)(C3)C4)c2nc1C] |
| 171 | 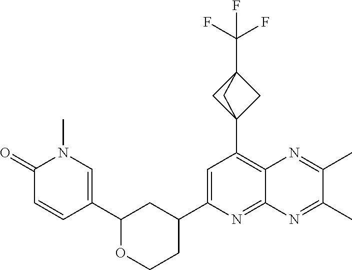 [SMILES: Cc1nc2nc(C3CCOC(c4ccc(=O)n(C)c4)C3)cc(C34CC(C(F)(F)F)(C3)C4)c2nc1C] |
| 173 | 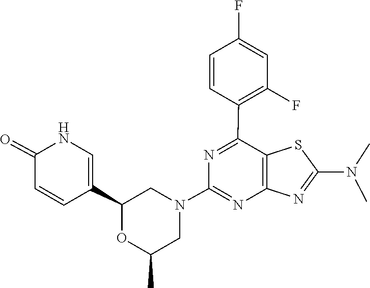 [SMILES: C[C@@H]1CN(c2nc(-c3ccc(F)cc3F)c3sc(N(C)C)nc3n2)C[C@H](c2ccc(=O)[nH]c2)O1] |
| 174 | 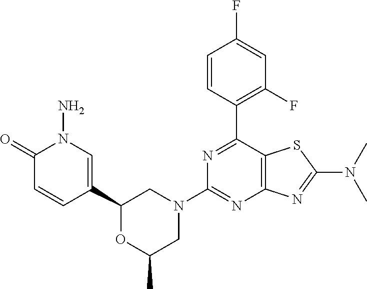 [SMILES: C[C@@H]1CN(c2nc(-c3ccc(F)cc3F)c3sc(N(C)C)nc3n2)C[C@H](c2ccc(=O)n(N)c2)O1] |
| 175 | 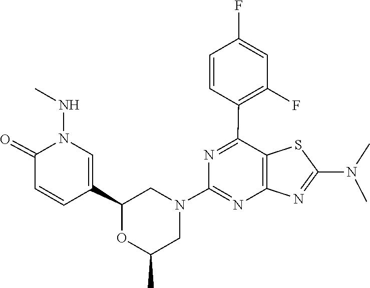 [SMILES: CNn1cc([C@H]2CN(c3nc(-c4ccc(F)cc4F)c4sc(N(C)C)nc4n3)C[C@@H](C)O2)ccc1=O] |
| 176 | 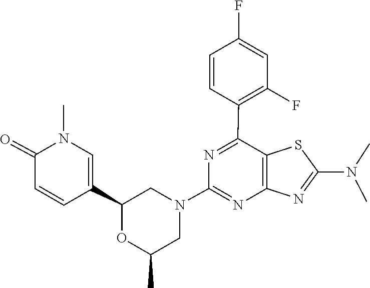 [SMILES: C[C@@H]1CN(c2nc(-c3ccc(F)cc3F)c3sc(N(C)C)nc3n2)C[C@H](c2ccc(=O)n(C)c2)O1] |
| 177 | 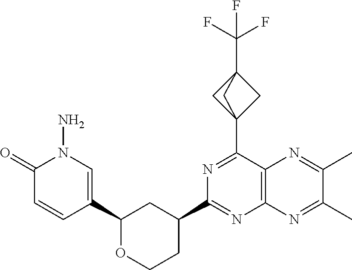 [SMILES: Cc1nc2nc([C@H]3CCO[C@@H](c4ccc(=O)n(N)c4)C3)nc(C34CC(C(F)(F)F)(C3)C4)c2nc1C] |
| 178 | 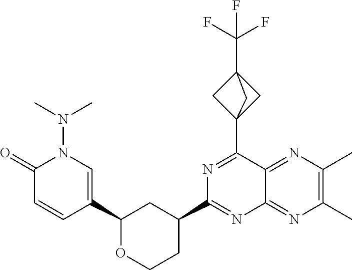 [SMILES: Cc1nc2nc([C@H]3CCO[C@@H](c4ccc(=O)n(N(C)C)c4)C3)nc(C34CC(C(F)(F)F)(C3)C4)c2nc1C] |
| 179 | 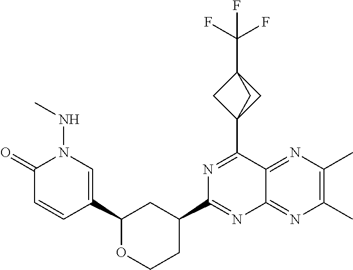 [SMILES: CNn1cc([C@H]2C[C@@H](c3nc(C45CC(C(F)(F)F)(C4)C5)c4nc(C)c(C)nc4n3)CCO2)ccc1=O] |
| 180 | 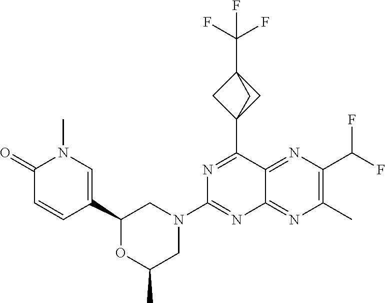 [SMILES: Cc1nc2nc(N3C[C@@H](C)O[C@@H](c4ccc(=O)n(C)c4)C3)nc(C34CC(C(F)(F)F)(C3)C4)c2nc1C(F)F] |
| 181 | 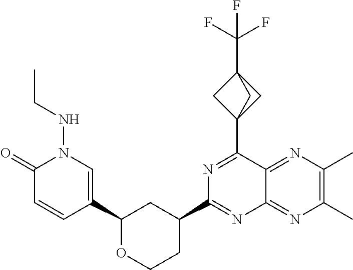 [SMILES: CCNn1cc([C@H]2C[C@@H](c3nc(C45CC(C(F)(F)F)(C4)C5)c4nc(C)c(C)nc4n3)CCO2)ccc1=O] |
| 182 | 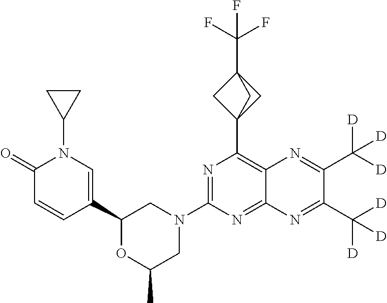 [SMILES: [2H]C([2H])([2H])c1nc2nc(N3C[C@@H](C)O[C@@H](c4ccc(=O)n(C5CC5)c4)C3)nc(C34CC(C(F)(F)F)(C3)C4)c2nc1C([2H])([2H])[2H]] |
| 183 | 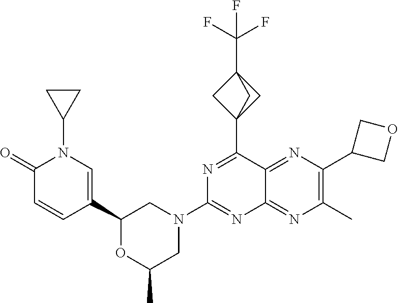 [SMILES: Cc1nc2nc(N3C[C@@H](C)O[C@@H](c4ccc(=O)n(C5CC5)c4)C3)nc(C34CC(C(F)(F)F)(C3)C4)c2nc1C1COC1] |
| 184 | 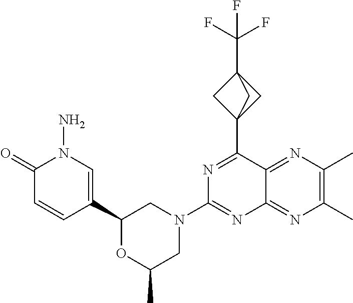 [SMILES: Cc1nc2nc(N3C[C@@H](C)O[C@@H](c4ccc(=O)n(N)c4)C3)nc(C34CC(C(F)(F)F)(C3)C4)c2nc1C] |
| 185 | 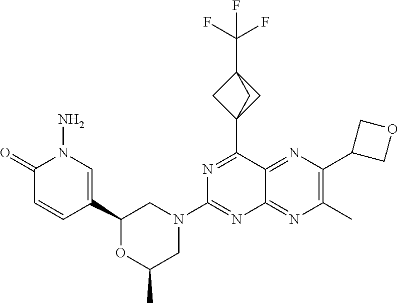 [SMILES: Cc1nc2nc(N3C[C@@H](C)O[C@@H](c4ccc(=O)n(N)c4)C3)nc(C34CC(C(F)(F)F)(C3)C4)c2nc1C1COC1] |
| 186 | 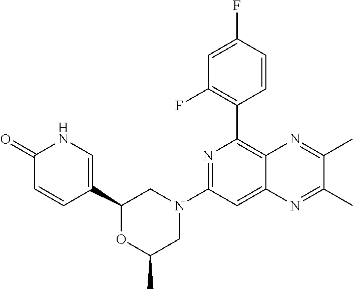 [SMILES: Cc1nc2cc(N3C[C@@H](C)O[C@@H](c4ccc(=O)[nH]c4)C3)nc(-c3ccc(F)cc3F)c2nc1C] |
| 187 | 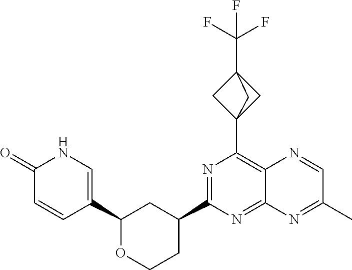 [SMILES: Cc1cnc2c(C34CC(C(F)(F)F)(C3)C4)nc([C@H]3CCO[C@@H](c4ccc(=O)[nH]c4)C3)nc2n1] |
| 188 | 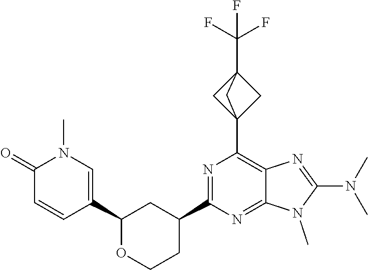 [SMILES: CN(C)c1nc2c(C34CC(C(F)(F)F)(C3)C4)nc([C@H]3CCO[C@@H](c4ccc(=O)n(C)c4)C3)nc2n1C] |
| 189 | 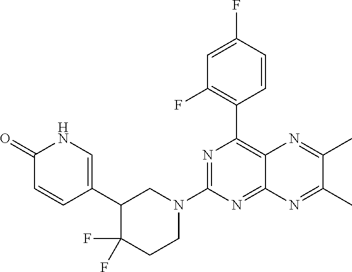 [SMILES: Cc1nc2nc(N3CCC(F)(F)C(c4ccc(=O)[nH]c4)C3)nc(-c3ccc(F)cc3F)c2nc1C] |
| 190 | 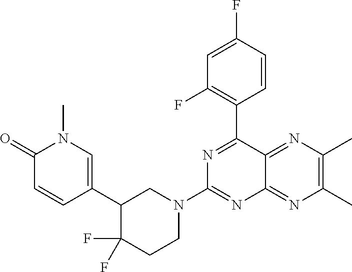 [SMILES: Cc1nc2nc(N3CCC(F)(F)C(c4ccc(=O)n(C)c4)C3)nc(-c3ccc(F)cc3F)c2nc1C] |
| 191 | 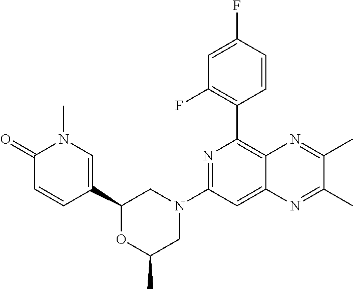 [SMILES: Cc1nc2cc(N3C[C@@H](C)O[C@@H](c4ccc(=O)n(C)c4)C3)nc(-c3ccc(F)cc3F)c2nc1C] |
| 192 | 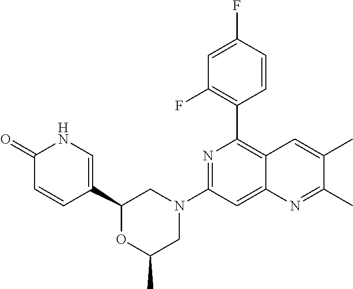 [SMILES: Cc1cc2c(-c3ccc(F)cc3F)nc(N3C[C@@H](C)O[C@@H](c4ccc(=O)[nH]c4)C3)cc2nc1C] |
| 193 | 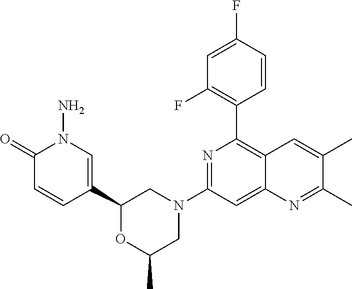 [SMILES: Cc1cc2c(-c3ccc(F)cc3F)nc(N3C[C@@H](C)O[C@@H](c4ccc(=O)n(N)c4)C3)cc2nc1C] |
| 194 | 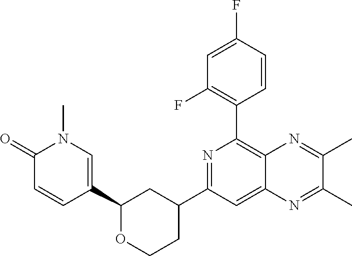 [SMILES: Cc1nc2cc(C3CCO[C@@H](c4ccc(=O)n(C)c4)C3)nc(-c3ccc(F)cc3F)c2nc1C] |
| 195 | 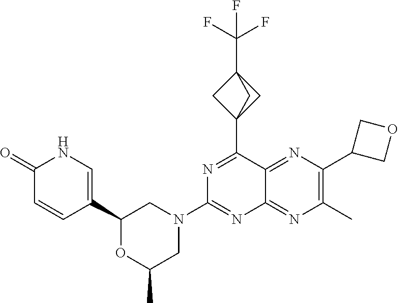 [SMILES: Cc1nc2nc(N3C[C@@H](C)O[C@@H](c4ccc(=O)[nH]c4)C3)nc(C34CC(C(F)(F)F)(C3)C4)c2nc1C1COC1] |
| 196 | 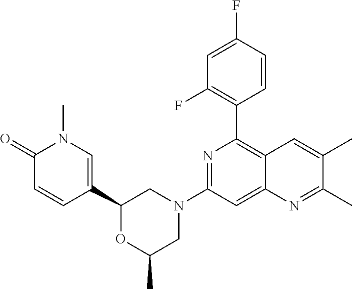 [SMILES: Cc1cc2c(-c3ccc(F)cc3F)nc(N3C[C@@H](C)O[C@@H](c4ccc(=O)n(C)c4)C3)cc2nc1C] |
| 197 | 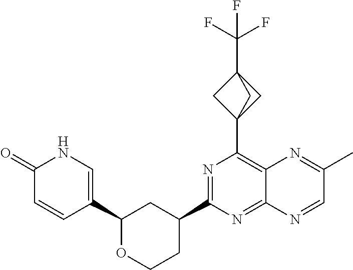 [SMILES: Cc1cnc2nc([C@H]3CCO[C@@H](c4ccc(=O)[nH]c4)C3)nc(C34CC(C(F)(F)F)(C3)C4)c2n1] |
| 198 | 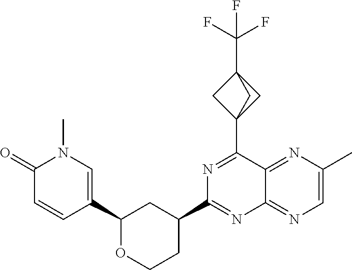 [SMILES: Cc1cnc2nc([C@H]3CCO[C@@H](c4ccc(=O)n(C)c4)C3)nc(C34CC(C(F)(F)F)(C3)C4)c2n1] |
| 201 | 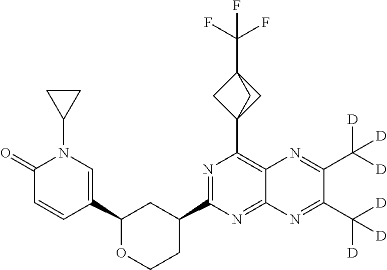 [SMILES: [2H]C([2H])([2H])c1nc2nc([C@H]3CCO[C@@H](c4ccc(=O)n(C5CC5)c4)C3)nc(C34CC(C(F)(F)F)(C3)C4)c2nc1C([2H])([2H])[2H]] |
| 202 | 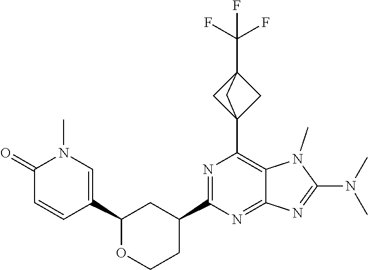 [SMILES: CN(C)c1nc2nc([C@H]3CCO[C@@H](c4ccc(=O)n(C)c4)C3)nc(C34CC(C(F)(F)F)(C3)C4)c2n1C] |
| 203 | 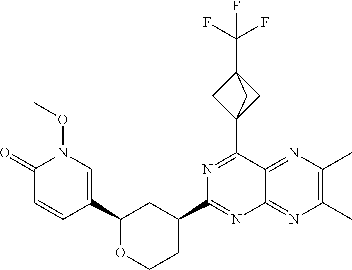 [SMILES: COn1cc([C@H]2C[C@@H](c3nc(C45CC(C(F)(F)F)(C4)C5)c4nc(C)c(C)nc4n3)CCO2)ccc1=O] |
| 204 | 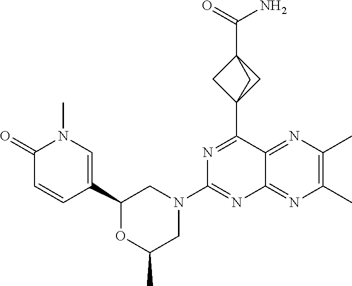 [SMILES: Cc1nc2nc(N3C[C@@H](C)O[C@@H](c4ccc(=O)n(C)c4)C3)nc(C34CC(C(N)=O)(C3)C4)c2nc1C] |
| 205 | 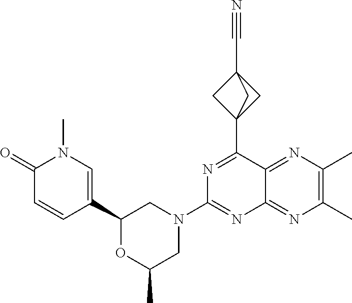 [SMILES: Cc1nc2nc(N3C[C@@H](C)O[C@@H](c4ccc(=O)n(C)c4)C3)nc(C34CC(C#N)(C3)C4)c2nc1C] |
| 206 | 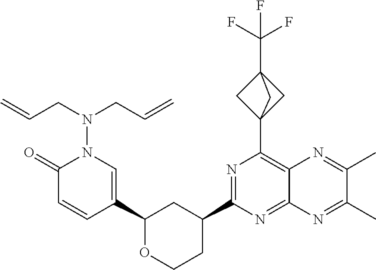 [SMILES: C=CCN(CC=C)n1cc([C@H]2C[C@@H](c3nc(C45CC(C(F)(F)F)(C4)C5)c4nc(C)c(C)nc4n3)CCO2)ccc1=O] |
| 207 | 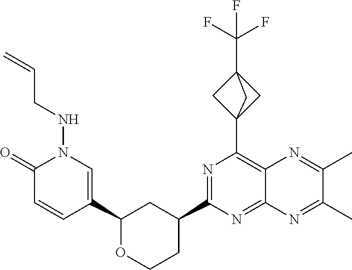 [SMILES: C=CCNn1cc([C@H]2C[C@@H](c3nc(C45CC(C(F)(F)F)(C4)C5)c4nc(C)c(C)nc4n3)CCO2)ccc1=O] |
| 208 | 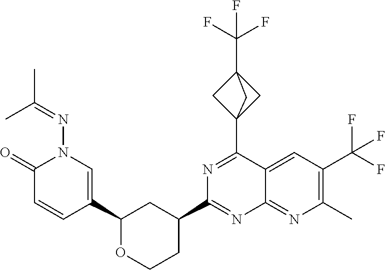 [SMILES: CC(C)=Nn1cc([C@H]2C[C@@H](c3nc(C45CC(C(F)(F)F)(C4)C5)c4cc(C(F)(F)F)c(C)nc4n3)CCO2)ccc1=O] |
| 209 | 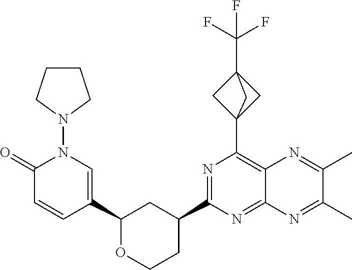 [SMILES: Cc1nc2nc([C@H]3CCO[C@@H](c4ccc(=O)n(N5CCCC5)c4)C3)nc(C34CC(C(F)(F)F)(C3)C4)c2nc1C] |
| 210 | 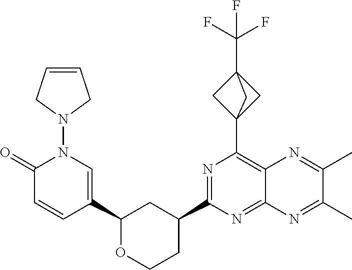 [SMILES: Cc1nc2nc([C@H]3CCO[C@@H](c4ccc(=O)n(N5CC=CC5)c4)C3)nc(C34CC(C(F)(F)F)(C3)C4)c2nc1C] |
| 211 | 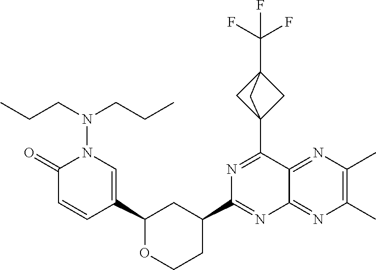 [SMILES: CCCN(CCC)n1cc([C@H]2C[C@@H](c3nc(C45CC(C(F)(F)F)(C4)C5)c4nc(C)c(C)nc4n3)CCO2)ccc1=O] |
| 212 | 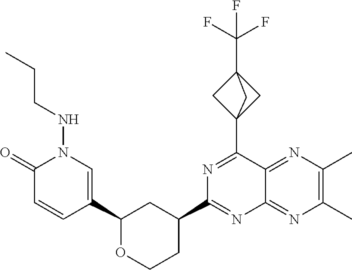 [SMILES: CCCNn1cc([C@H]2C[C@@H](c3nc(C45CC(C(F)(F)F)(C4)C5)c4nc(C)c(C)nc4n3)CCO2)ccc1=O] |
| 213 | 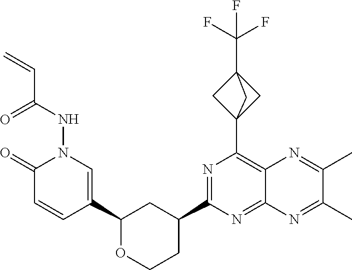 [SMILES: C=CC(=O)Nn1cc([C@H]2C[C@@H](c3nc(C45CC(C(F)(F)F)(C4)C5)c4nc(C)c(C)nc4n3)CCO2)ccc1=O] |
| 214 | 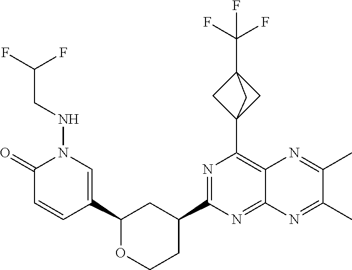 [SMILES: Cc1nc2nc([C@H]3CCO[C@@H](c4ccc(=O)n(NCC(F)F)c4)C3)nc(C34CC(C(F)(F)F)(C3)C4)c2nc1C] |
| 215 | 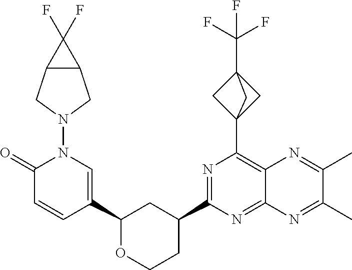 [SMILES: Cc1nc2nc([C@H]3CCO[C@@H](c4ccc(=O)n(N5CC6C(C5)C6(F)F)c4)C3)nc(C34CC(C(F)(F)F)(C3)C4)c2nc1C] |
| 216 | 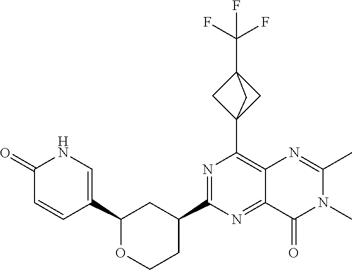 [SMILES: Cc1nc2c(C34CC(C(F)(F)F)(C3)C4)nc([C@H]3CCO[C@@H](c4ccc(=O)[nH]c4)C3)nc2c(=O)n1C] |
| 217 | 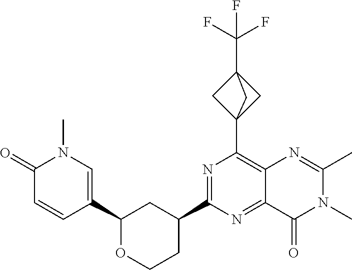 [SMILES: Cc1nc2c(C34CC(C(F)(F)F)(C3)C4)nc([C@H]3CCO[C@@H](c4ccc(=O)n(C)c4)C3)nc2c(=O)n1C] |
| 218 | 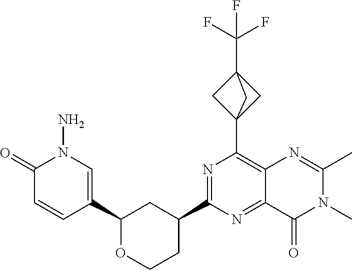 [SMILES: Cc1nc2c(C34CC(C(F)(F)F)(C3)C4)nc([C@H]3CCO[C@@H](c4ccc(=O)n(N)c4)C3)nc2c(=O)n1C] |
| 219 | 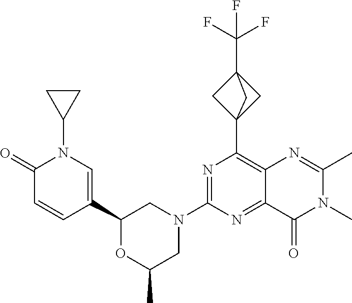 [SMILES: Cc1nc2c(C34CC(C(F)(F)F)(C3)C4)nc(N3C[C@@H](C)O[C@@H](c4ccc(=O)n(C5CC5)c4)C3)nc2c(=O)n1C] |
| 220 | 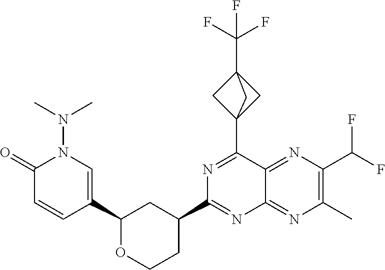 [SMILES: Cc1nc2nc([C@H]3CCO[C@@H](c4ccc(=O)n(N(C)C)c4)C3)nc(C34CC(C(F)(F)F)(C3)C4)c2nc1C(F)F] |
| 221 | 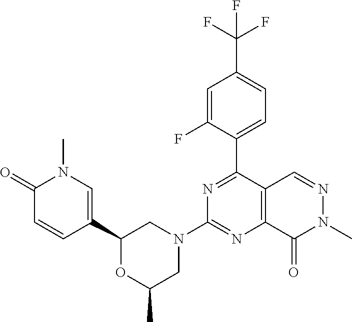 [SMILES: C[C@@H]1CN(c2nc(-c3ccc(C(F)(F)F)cc3F)c3cnn(C)c(=O)c3n2)C[C@H](c2ccc(=O)n(C)c2)O1] |
| 222 | 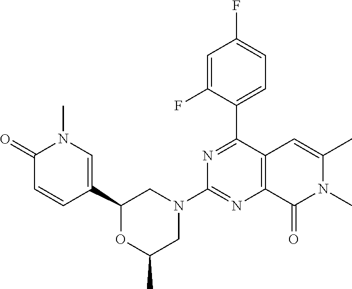 [SMILES: Cc1cc2c(-c3ccc(F)cc3F)nc(N3C[C@@H](C)O[C@@H](c4ccc(=O)n(C)c4)C3)nc2c(=O)n1C] |
| 223 | 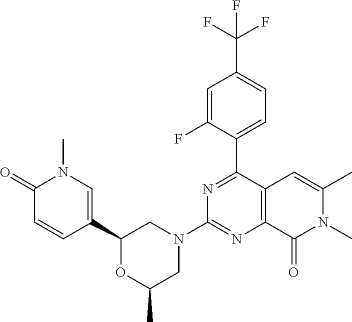 [SMILES: Cc1cc2c(-c3ccc(C(F)(F)F)cc3F)nc(N3C[C@@H](C)O[C@@H](c4ccc(=O)n(C)c4)C3)nc2c(=O)n1C] |
| 224 | 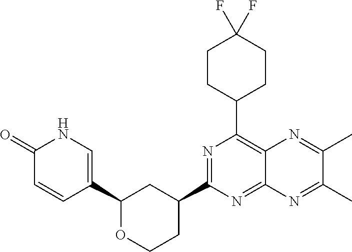 [SMILES: Cc1nc2nc([C@H]3CCO[C@@H](c4ccc(=O)[nH]c4)C3)nc(C3CCC(F)(F)CC3)c2nc1C] |
| 225 | 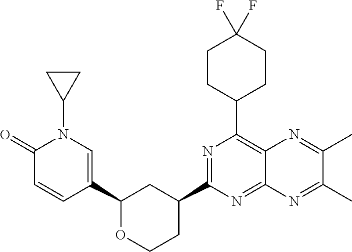 [SMILES: Cc1nc2nc([C@H]3CCO[C@@H](c4ccc(=O)n(C5CC5)c4)C3)nc(C3CCC(F)(F)CC3)c2nc1C] |
| 226 | 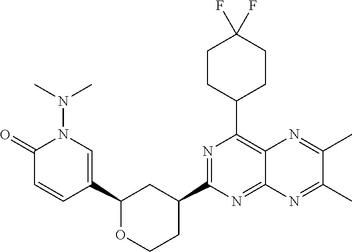 [SMILES: Cc1nc2nc([C@H]3CCO[C@@H](c4ccc(=O)n(N(C)C)c4)C3)nc(C3CCC(F)(F)CC3)c2nc1C] |
| 227 | 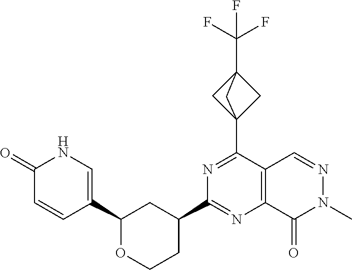 [SMILES: Cn1ncc2c(C34CC(C(F)(F)F)(C3)C4)nc([C@H]3CCO[C@@H](c4ccc(=O)[nH]c4)C3)nc2c1=O] |
| 228 | 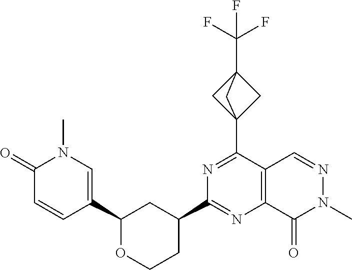 [SMILES: Cn1cc([C@H]2C[C@@H](c3nc(C45CC(C(F)(F)F)(C4)C5)c4cnn(C)c(=O)c4n3)CCO2)ccc1=O] |
| 229 | 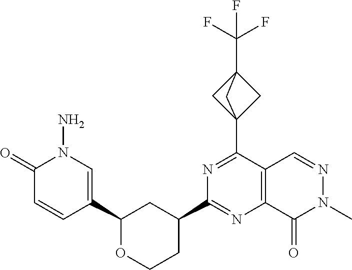 [SMILES: Cn1ncc2c(C34CC(C(F)(F)F)(C3)C4)nc([C@H]3CCO[C@@H](c4ccc(=O)n(N)c4)C3)nc2c1=O] |
| 230 | 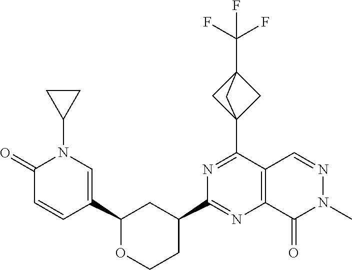 [SMILES: Cn1ncc2c(C34CC(C(F)(F)F)(C3)C4)nc([C@H]3CCO[C@@H](c4ccc(=O)n(C5CC5)c4)C3)nc2c1=O] |
| 231 | 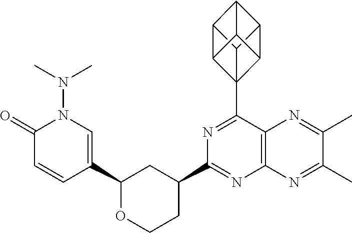 [SMILES: Cc1nc2nc([C@H]3CCO[C@@H](c4ccc(=O)n(N(C)C)c4)C3)nc(C34C5C6C7C5C3C7C64)c2nc1C] |
| 232 | 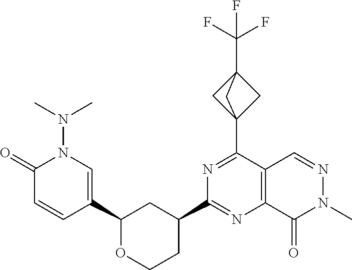 [SMILES: CN(C)n1cc([C@H]2C[C@@H](c3nc(C45CC(C(F)(F)F)(C4)C5)c4cnn(C)c(=O)c4n3)CCO2)ccc1=O] |
| 233 | 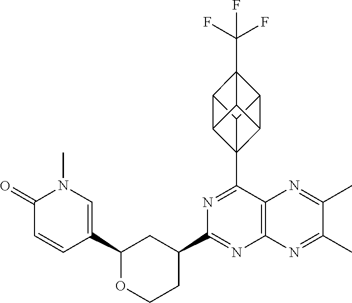 [SMILES: Cc1nc2nc([C@H]3CCO[C@@H](c4ccc(=O)n(C)c4)C3)nc(C34C5C6C3C3C4C5C63C(F)(F)F)c2nc1C] |
| 234 | 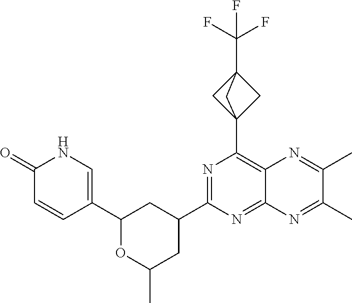 [SMILES: Cc1nc2nc(C3CC(C)OC(c4ccc(=O)[nH]c4)C3)nc(C34CC(C(F)(F)F)(C3)C4)c2nc1C] |
| 235 | 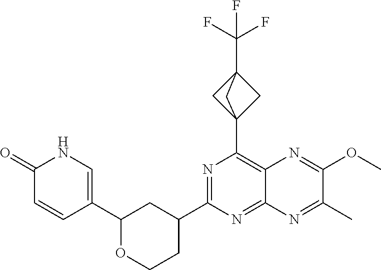 [SMILES: COc1nc2c(C34CC(C(F)(F)F)(C3)C4)nc(C3CCOC(c4ccc(=O)[nH]c4)C3)nc2nc1C] |
| 236 | 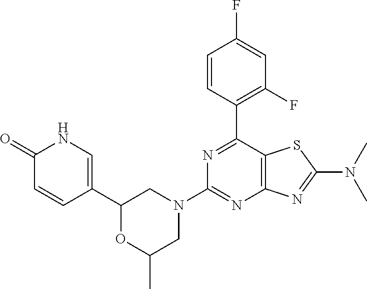 [SMILES: CC1CN(c2nc(-c3ccc(F)cc3F)c3sc(N(C)C)nc3n2)CC(c2ccc(=O)[nH]c2)O1] |
| 237 | 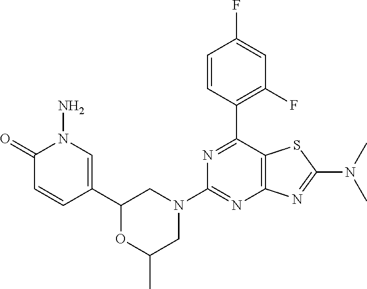 [SMILES: CC1CN(c2nc(-c3ccc(F)cc3F)c3sc(N(C)C)nc3n2)CC(c2ccc(=O)n(N)c2)O1] |
| 238 | 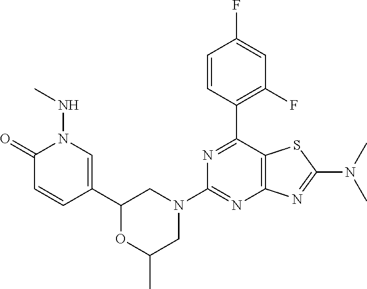 [SMILES: CNn1cc(C2CN(c3nc(-c4ccc(F)cc4F)c4sc(N(C)C)nc4n3)CC(C)O2)ccc1=O] |
| 239 | 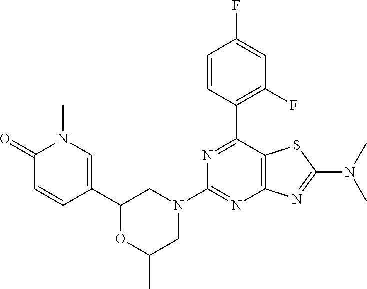 [SMILES: CC1CN(c2nc(-c3ccc(F)cc3F)c3sc(N(C)C)nc3n2)CC(c2ccc(=O)n(C)c2)O1] |
| 240 | 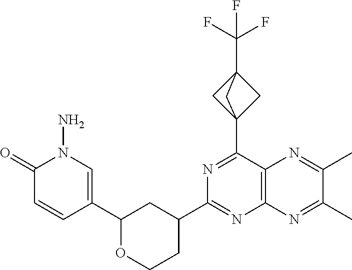 [SMILES: Cc1nc2nc(C3CCOC(c4ccc(=O)n(N)c4)C3)nc(C34CC(C(F)(F)F)(C3)C4)c2nc1C] |
| 241 | 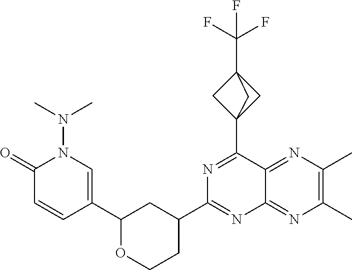 [SMILES: Cc1nc2nc(C3CCOC(c4ccc(=O)n(N(C)C)c4)C3)nc(C34CC(C(F)(F)F)(C3)C4)c2nc1C] |
| 242 | 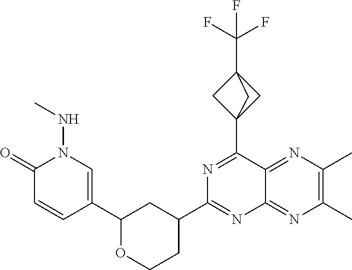 [SMILES: CNn1cc(C2CC(c3nc(C45CC(C(F)(F)F)(C4)C5)c4nc(C)c(C)nc4n3)CCO2)ccc1=O] |
| 243 | 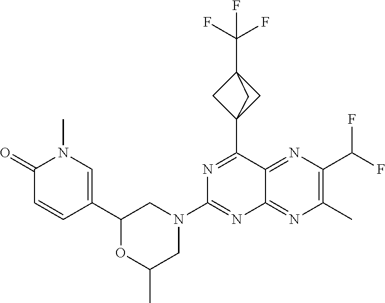 [SMILES: Cc1nc2nc(N3CC(C)OC(c4ccc(=O)n(C)c4)C3)nc(C34CC(C(F)(F)F)(C3)C4)c2nc1C(F)F] |
| 244 | 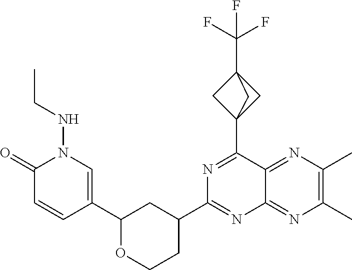 [SMILES: CCNn1cc(C2CC(c3nc(C45CC(C(F)(F)F)(C4)C5)c4nc(C)c(C)nc4n3)CCO2)ccc1=O] |
| 245 | 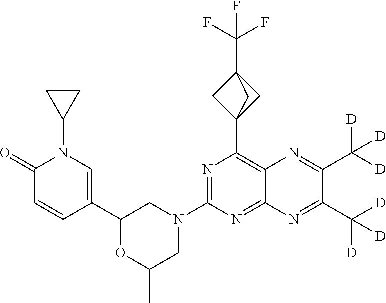 [SMILES: [2H]C([2H])([2H])c1nc2nc(N3CC(C)OC(c4ccc(=O)n(C5CC5)c4)C3)nc(C34CC(C(F)(F)F)(C3)C4)c2nc1C([2H])([2H])[2H]] |
| 246 | 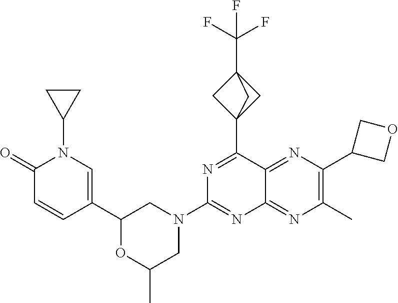 [SMILES: Cc1nc2nc(N3CC(C)OC(c4ccc(=O)n(C5CC5)c4)C3)nc(C34CC(C(F)(F)F)(C3)C4)c2nc1C1COC1] |
| 247 | 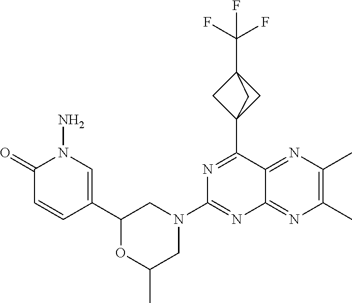 [SMILES: Cc1nc2nc(N3CC(C)OC(c4ccc(=O)n(N)c4)C3)nc(C34CC(C(F)(F)F)(C3)C4)c2nc1C] |
| 248 | 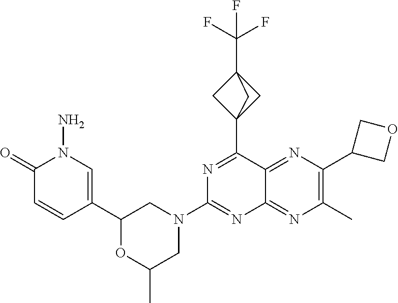 [SMILES: Cc1nc2nc(N3CC(C)OC(c4ccc(=O)n(N)c4)C3)nc(C34CC(C(F)(F)F)(C3)C4)c2nc1C1COC1] |
| 249 | 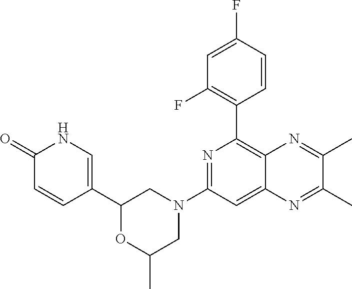 [SMILES: Cc1nc2cc(N3CC(C)OC(c4ccc(=O)[nH]c4)C3)nc(-c3ccc(F)cc3F)c2nc1C] |
| 250 | 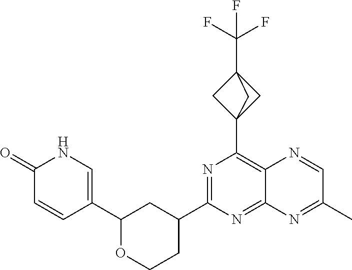 [SMILES: Cc1cnc2c(C34CC(C(F)(F)F)(C3)C4)nc(C3CCOC(c4ccc(=O)[nH]c4)C3)nc2n1] |
| 251 | 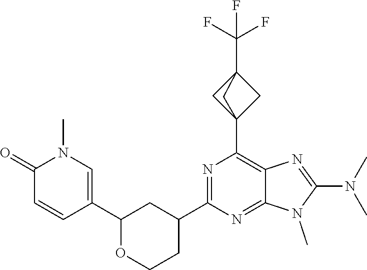 [SMILES: CN(C)c1nc2c(C34CC(C(F)(F)F)(C3)C4)nc(C3CCOC(c4ccc(=O)n(C)c4)C3)nc2n1C] |
| 252 | 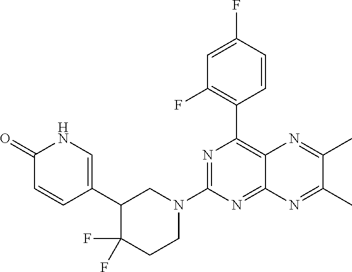 [SMILES: Cc1nc2nc(N3CCC(F)(F)C(c4ccc(=O)[nH]c4)C3)nc(-c3ccc(F)cc3F)c2nc1C] |
| 253 | 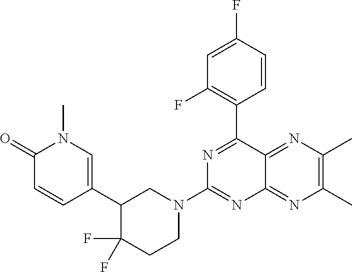 [SMILES: Cc1nc2nc(N3CCC(F)(F)C(c4ccc(=O)n(C)c4)C3)nc(-c3ccc(F)cc3F)c2nc1C] |
| 254 | 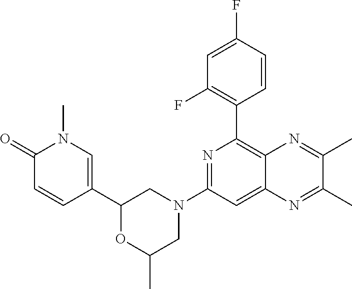 [SMILES: Cc1nc2cc(N3CC(C)OC(c4ccc(=O)n(C)c4)C3)nc(-c3ccc(F)cc3F)c2nc1C] |
| 255 | 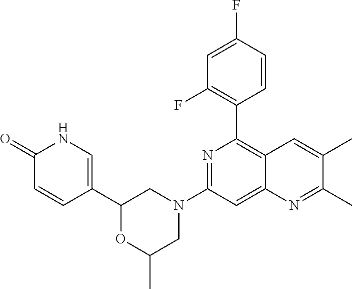 [SMILES: Cc1cc2c(-c3ccc(F)cc3F)nc(N3CC(C)OC(c4ccc(=O)[nH]c4)C3)cc2nc1C] |
| 256 | 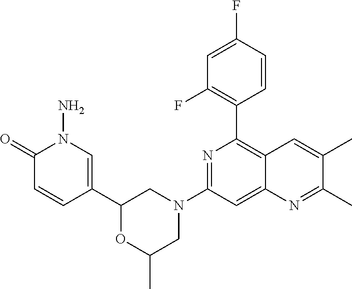 [SMILES: Cc1cc2c(-c3ccc(F)cc3F)nc(N3CC(C)OC(c4ccc(=O)n(N)c4)C3)cc2nc1C] |
| 257 | 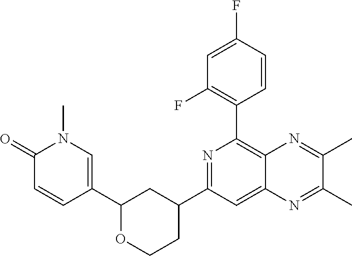 [SMILES: Cc1nc2cc(C3CCOC(c4ccc(=O)n(C)c4)C3)nc(-c3ccc(F)cc3F)c2nc1C] |
| 258 | 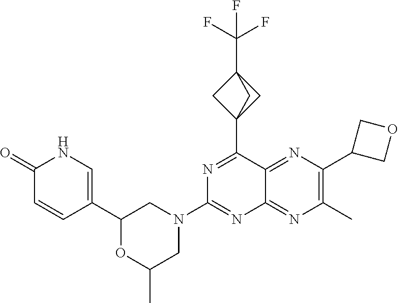 [SMILES: Cc1nc2nc(N3CC(C)OC(c4ccc(=O)[nH]c4)C3)nc(C34CC(C(F)(F)F)(C3)C4)c2nc1C1COC1] |
| 259 | 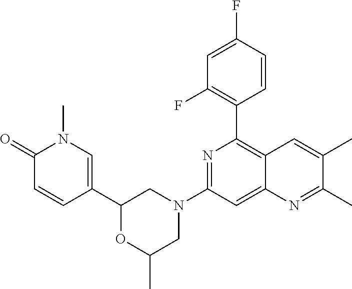 [SMILES: Cc1cc2c(-c3ccc(F)cc3F)nc(N3CC(C)OC(c4ccc(=O)n(C)c4)C3)cc2nc1C] |
| 260 | 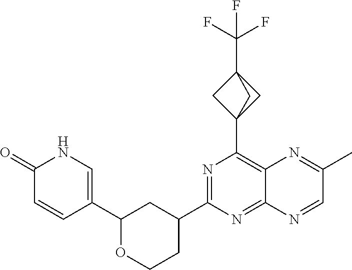 [SMILES: Cc1cnc2nc(C3CCOC(c4ccc(=O)[nH]c4)C3)nc(C34CC(C(F)(F)F)(C3)C4)c2n1] |
| 261 | 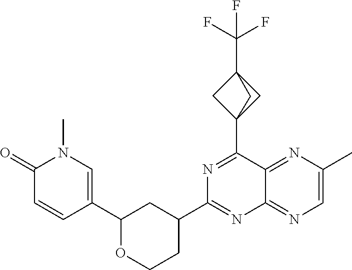 [SMILES: Cc1cnc2nc(C3CCOC(c4ccc(=O)n(C)c4)C3)nc(C34CC(C(F)(F)F)(C3)C4)c2n1] |
| 262 | 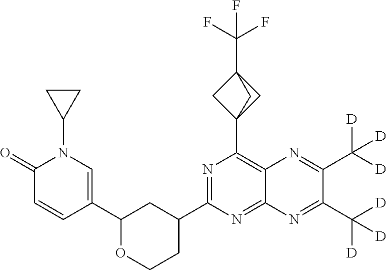 [SMILES: [2H]C([2H])([2H])c1nc2nc(C3CCOC(c4ccc(=O)n(C5CC5)c4)C3)nc(C34CC(C(F)(F)F)(C3)C4)c2nc1C([2H])([2H])[2H]] |
| 263 | 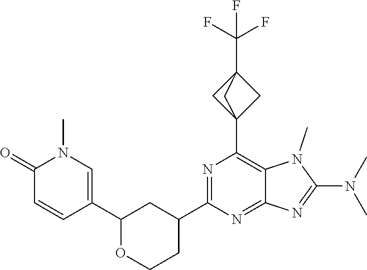 [SMILES: CN(C)c1nc2nc(C3CCOC(c4ccc(=O)n(C)c4)C3)nc(C34CC(C(F)(F)F)(C3)C4)c2n1C] |
| 264 | 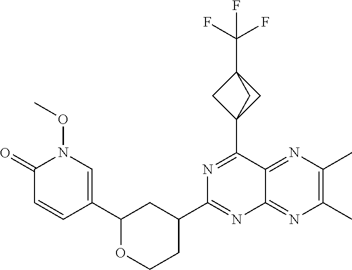 [SMILES: COn1cc(C2CC(c3nc(C45CC(C(F)(F)F)(C4)C5)c4nc(C)c(C)nc4n3)CCO2)ccc1=O] |
| 265 | 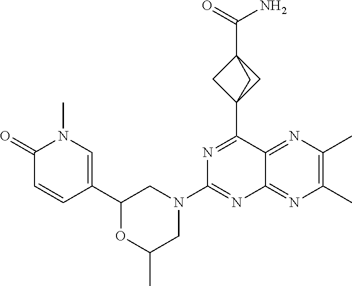 [SMILES: Cc1nc2nc(N3CC(C)OC(c4ccc(=O)n(C)c4)C3)nc(C34CC(C(N)=O)(C3)C4)c2nc1C] |
| 266 | 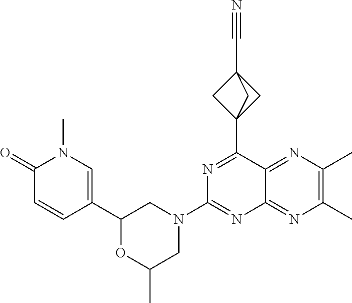 [SMILES: Cc1nc2nc(N3CC(C)OC(c4ccc(=O)n(C)c4)C3)nc(C34CC(C#N)(C3)C4)c2nc1C] |
| 267 | 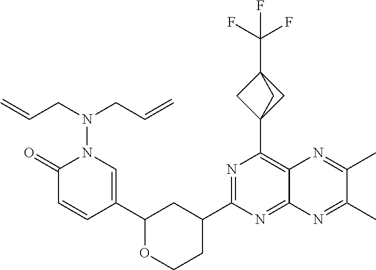 [SMILES: C=CCN(CC=C)n1cc(C2CC(c3nc(C45CC(C(F)(F)F)(C4)C5)c4nc(C)c(C)nc4n3)CCO2)ccc1=O] |
| 268 | 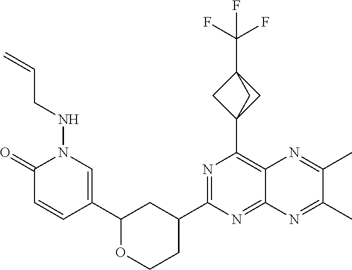 [SMILES: C=CCNn1cc(C2CC(c3nc(C45CC(C(F)(F)F)(C4)C5)c4nc(C)c(C)nc4n3)CCO2)ccc1=O] |
| 269 | 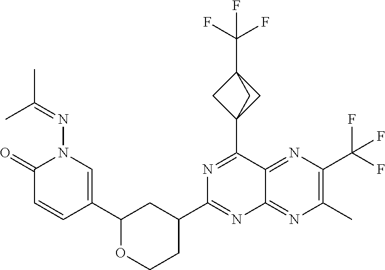 [SMILES: CC(C)=Nn1cc(C2CC(c3nc(C45CC(C(F)(F)F)(C4)C5)c4nc(C(F)(F)F)c(C)nc4n3)CCO2)ccc1=O] |
| 270 | 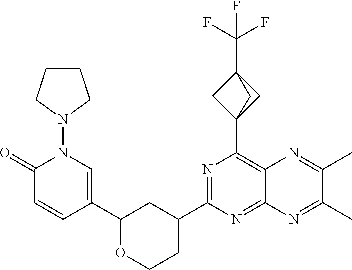 [SMILES: Cc1nc2nc(C3CCOC(c4ccc(=O)n(N5CCCC5)c4)C3)nc(C34CC(C(F)(F)F)(C3)C4)c2nc1C] |
| 271 | 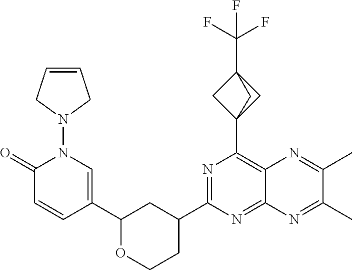 [SMILES: Cc1nc2nc(C3CCOC(c4ccc(=O)n(N5CC=CC5)c4)C3)nc(C34CC(C(F)(F)F)(C3)C4)c2nc1C] |
| 272 | 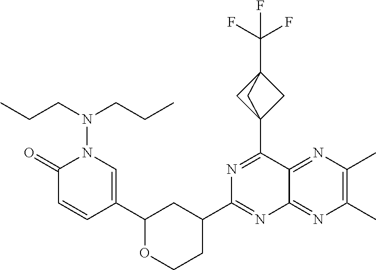 [SMILES: CCCN(CCC)n1cc(C2CC(c3nc(C45CC(C(F)(F)F)(C4)C5)c4nc(C)c(C)nc4n3)CCO2)ccc1=O] |
| 273 | 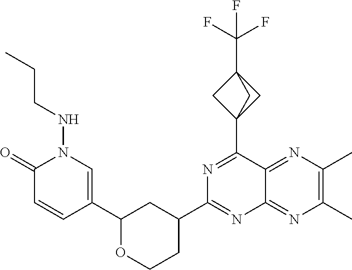 [SMILES: CCCNn1cc(C2CC(c3nc(C45CC(C(F)(F)F)(C4)C5)c4nc(C)c(C)nc4n3)CCO2)ccc1=O] |
| 274 | 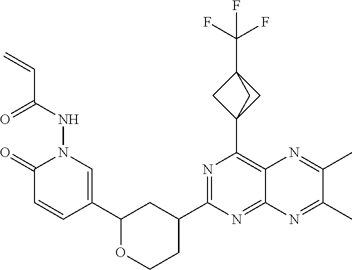 [SMILES: C=CC(=O)Nn1cc(C2CC(c3nc(C45CC(C(F)(F)F)(C4)C5)c4nc(C)c(C)nc4n3)CCO2)ccc1=O] |
| 275 | 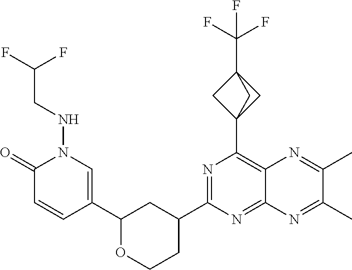 [SMILES: Cc1nc2nc(C3CCOC(c4ccc(=O)n(NCC(F)F)c4)C3)nc(C34CC(C(F)(F)F)(C3)C4)c2nc1C] |
| 276 | 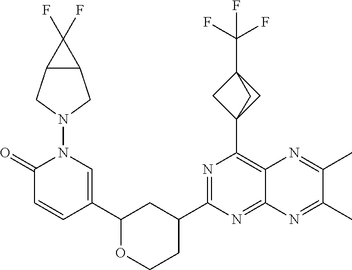 [SMILES: Cc1nc2nc(C3CCOC(c4ccc(=O)n(N5CC6C(C5)C6(F)F)c4)C3)nc(C34CC(C(F)(F)F)(C3)C4)c2nc1C] |
| 277 | 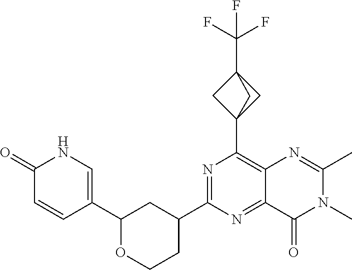 [SMILES: Cc1nc2c(C34CC(C(F)(F)F)(C3)C4)nc(C3CCOC(c4ccc(=O)[nH]c4)C3)nc2c(=O)n1C] |
| 278 | 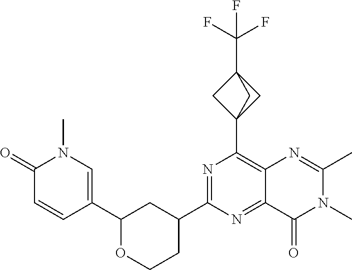 [SMILES: Cc1nc2c(C34CC(C(F)(F)F)(C3)C4)nc(C3CCOC(c4ccc(=O)n(C)c4)C3)nc2c(=O)n1C] |
| 279 | 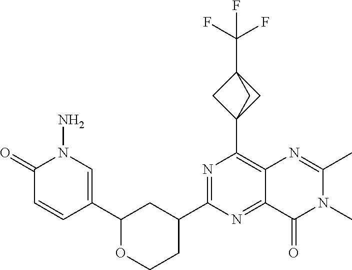 [SMILES: Cc1nc2c(C34CC(C(F)(F)F)(C3)C4)nc(C3CCOC(c4ccc(=O)n(N)c4)C3)nc2c(=O)n1C] |
| 280 | 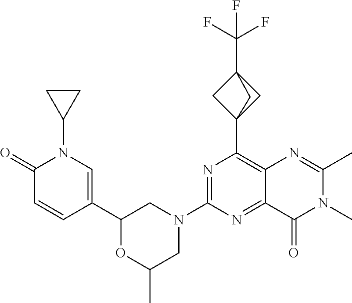 [SMILES: Cc1nc2c(C34CC(C(F)(F)F)(C3)C4)nc(N3CC(C)OC(c4ccc(=O)n(C5CC5)c4)C3)nc2c(=O)n1C] |
| 281 | 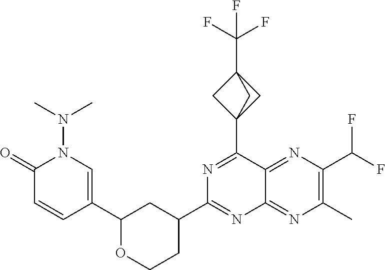 [SMILES: Cc1nc2nc(C3CCOC(c4ccc(=O)n(N(C)C)c4)C3)nc(C34CC(C(F)(F)F)(C3)C4)c2nc1C(F)F] |
| 282 | 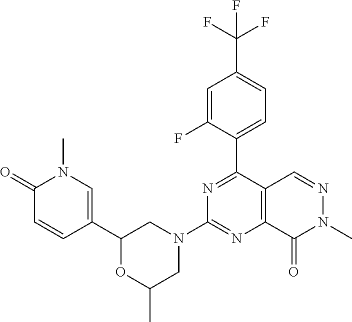 [SMILES: CC1CN(c2nc(-c3ccc(C(F)(F)F)cc3F)c3cnn(C)c(=O)c3n2)CC(c2ccc(=O)n(C)c2)O1] |
| 283 | 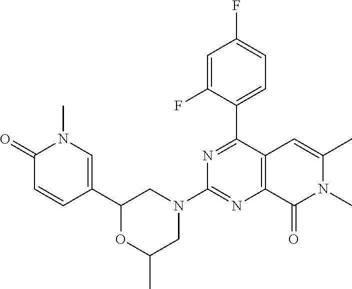 [SMILES: Cc1cc2c(-c3ccc(F)cc3F)nc(N3CC(C)OC(c4ccc(=O)n(C)c4)C3)nc2c(=O)n1C] |
| 284 | 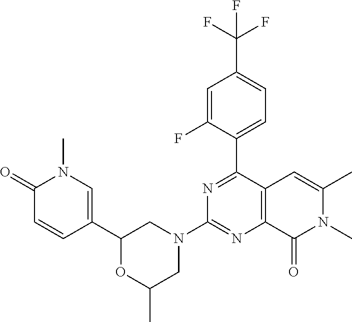 [SMILES: Cc1cc2c(-c3ccc(C(F)(F)F)cc3F)nc(N3CC(C)OC(c4ccc(=O)n(C)c4)C3)nc2c(=O)n1C] |
| 285 | 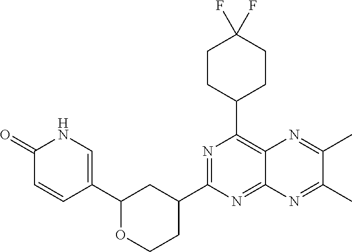 [SMILES: Cc1nc2nc(C3CCOC(c4ccc(=O)[nH]c4)C3)nc(C3CCC(F)(F)CC3)c2nc1C] |
| 286 | 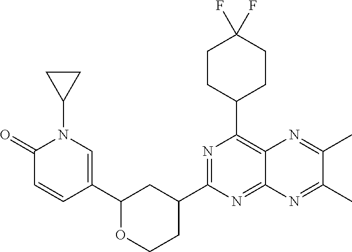 [SMILES: Cc1nc2nc(C3CCOC(c4ccc(=O)n(C5CC5)c4)C3)nc(C3CCC(F)(F)CC3)c2nc1C] |
| 287 | 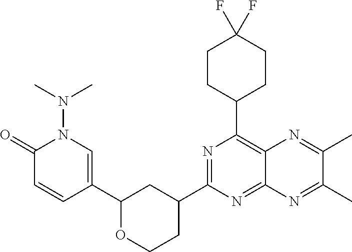 [SMILES: Cc1nc2nc(C3CCOC(c4ccc(=O)n(N(C)C)c4)C3)nc(C3CCC(F)(F)CC3)c2nc1C] |
| 288 | 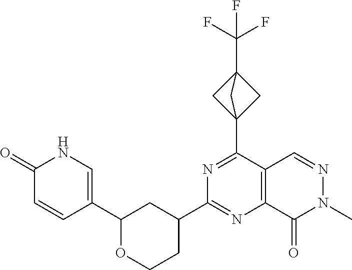 [SMILES: Cn1ncc2c(C34CC(C(F)(F)F)(C3)C4)nc(C3CCOC(c4ccc(=O)[nH]c4)C3)nc2c1=O] |
| 289 | 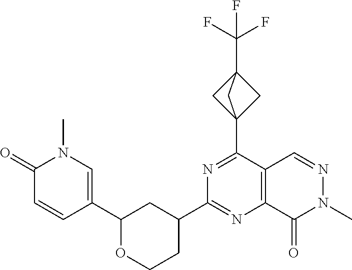 [SMILES: Cn1cc(C2CC(c3nc(C45CC(C(F)(F)F)(C4)C5)c4cnn(C)c(=O)c4n3)CCO2)ccc1=O] |
| 290 | 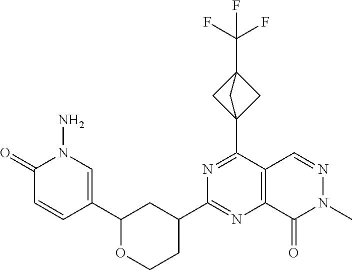 [SMILES: Cn1ncc2c(C34CC(C(F)(F)F)(C3)C4)nc(C3CCOC(c4ccc(=O)n(N)c4)C3)nc2c1=O] |
| 291 | 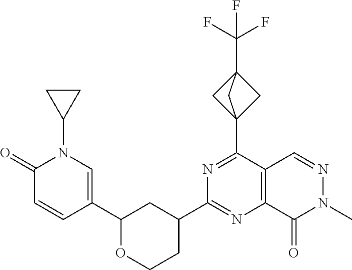 [SMILES: Cn1ncc2c(C34CC(C(F)(F)F)(C3)C4)nc(C3CCOC(c4ccc(=O)n(C5CC5)c4)C3)nc2c1=O] |
| 292 | 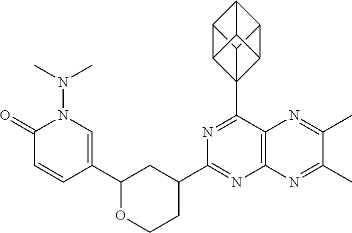 [SMILES: Cc1nc2nc(C3CCOC(c4ccc(=O)n(N(C)C)c4)C3)nc(C34C5C6C7C5C3C7C64)c2nc1C] |
| 293 | 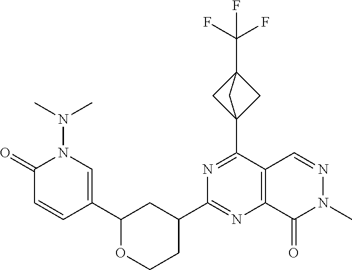 [SMILES: CN(C)n1cc(C2CC(c3nc(C45CC(C(F)(F)F)(C4)C5)c4cnn(C)c(=O)c4n3)CCO2)ccc1=O] |
| 294 | 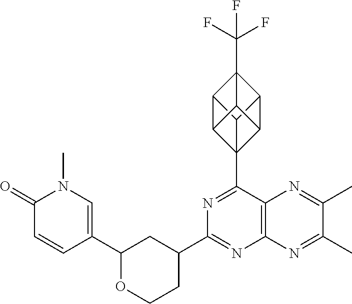 [SMILES: Cc1nc2nc(C3CCOC(c4ccc(=O)n(C)c4)C3)nc(C34C5C6C3C3C4C5C63C(F)(F)F)c2nc1C] |
| 295 | 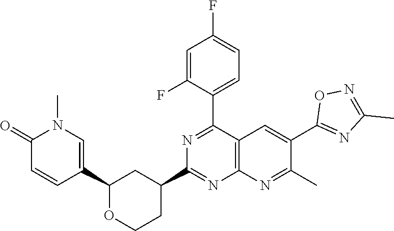 [SMILES: Cc1noc(-c2cc3c(-c4ccc(F)cc4F)nc([C@H]4CCO[C@@H](c5ccc(=O)n(C)c5)C4)nc3nc2C)n1] |
| 296 | 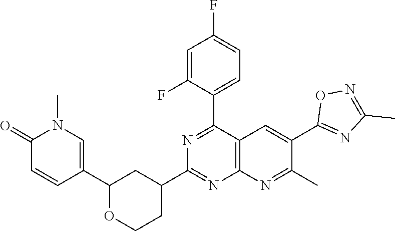 [SMILES: Cc1noc(-c2cc3c(-c4ccc(F)cc4F)nc(C4CCOC(c5ccc(=O)n(C)c5)C4)nc3nc2C)n1] |
| 297 | 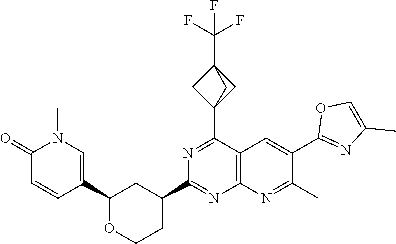 [SMILES: Cc1coc(-c2cc3c(C45CC(C(F)(F)F)(C4)C5)nc([C@H]4CCO[C@@H](c5ccc(=O)n(C)c5)C4)nc3nc2C)n1] |
| 298 | 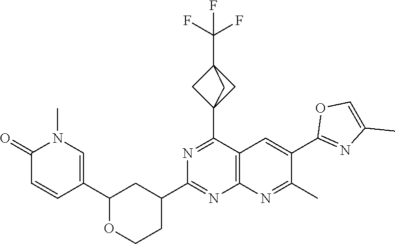 [SMILES: Cc1coc(-c2cc3c(C45CC(C(F)(F)F)(C4)C5)nc(C4CCOC(c5ccc(=O)n(C)c5)C4)nc3nc2C)n1] |
| 299 | 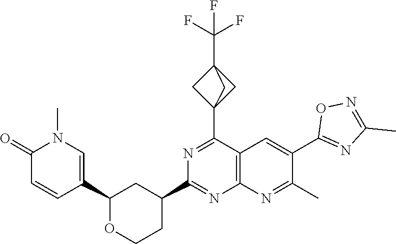 [SMILES: Cc1noc(-c2cc3c(C45CC(C(F)(F)F)(C4)C5)nc([C@H]4CCO[C@@H](c5ccc(=O)n(C)c5)C4)nc3nc2C)n1] |
| 300 | 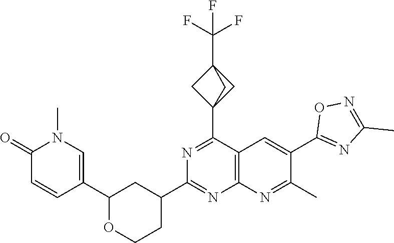 [SMILES: Cc1noc(-c2cc3c(C45CC(C(F)(F)F)(C4)C5)nc(C4CCOC(c5ccc(=O)n(C)c5)C4)nc3nc2C)n1] |
| 301 | 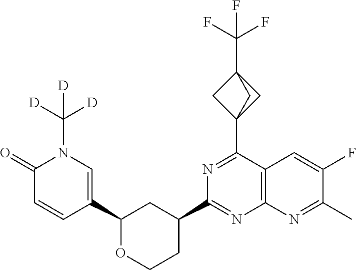 [SMILES: [2H]C([2H])([2H])n1cc([C@H]2C[C@@H](c3nc(C45CC(C(F)(F)F)(C4)C5)c4cc(F)c(C)nc4n3)CCO2)ccc1=O] |
| 302 | 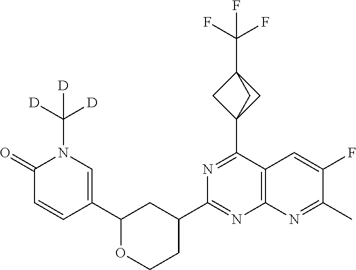 [SMILES: [2H]C([2H])([2H])n1cc(C2CC(c3nc(C45CC(C(F)(F)F)(C4)C5)c4cc(F)c(C)nc4n3)CCO2)ccc1=O] |
| 303 | 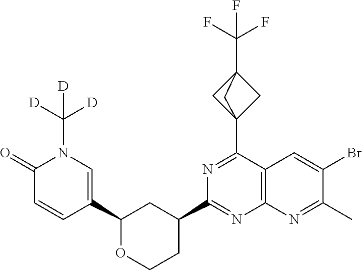 [SMILES: [2H]C([2H])([2H])n1cc([C@H]2C[C@@H](c3nc(C45CC(C(F)(F)F)(C4)C5)c4cc(Br)c(C)nc4n3)CCO2)ccc1=O] |
| 304 | 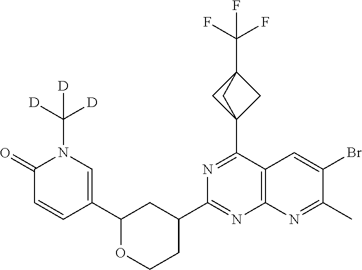 [SMILES: [2H]C([2H])([2H])n1cc(C2CC(c3nc(C45CC(C(F)(F)F)(C4)C5)c4cc(Br)c(C)nc4n3)CCO2)ccc1=O] |
| 305 | 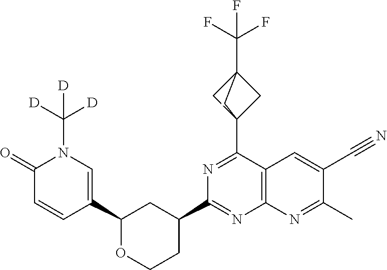 [SMILES: [2H]C([2H])([2H])n1cc([C@H]2C[C@@H](c3nc(C45CC(C(F)(F)F)(C4)C5)c4cc(C#N)c(C)nc4n3)CCO2)ccc1=O] |
| 306 | 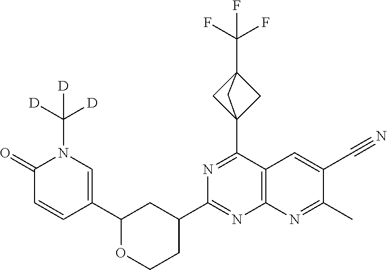 [SMILES: [2H]C([2H])([2H])n1cc(C2CC(c3nc(C45CC(C(F)(F)F)(C4)C5)c4cc(C#N)c(C)nc4n3)CCO2)ccc1=O] |
| 307 | 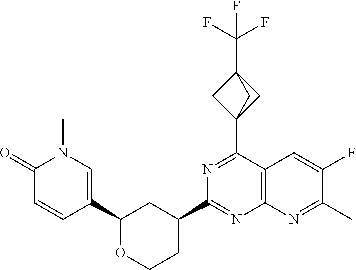 [SMILES: Cc1nc2nc([C@H]3CCO[C@@H](c4ccc(=O)n(C)c4)C3)nc(C34CC(C(F)(F)F)(C3)C4)c2cc1F] |
| 308 | 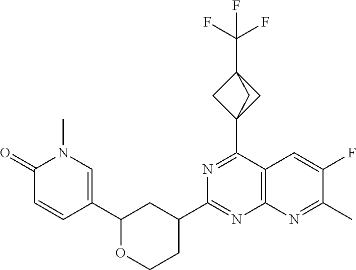 [SMILES: Cc1nc2nc(C3CCOC(c4ccc(=O)n(C)c4)C3)nc(C34CC(C(F)(F)F)(C3)C4)c2cc1F] |
| 309 | 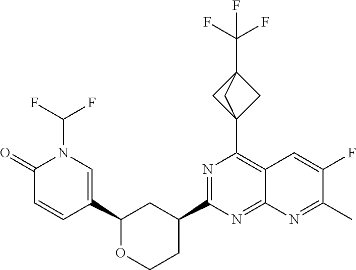 [SMILES: Cc1nc2nc([C@H]3CCO[C@@H](c4ccc(=O)n(C(F)F)c4)C3)nc(C34CC(C(F)(F)F)(C3)C4)c2cc1F] |
| 310 | 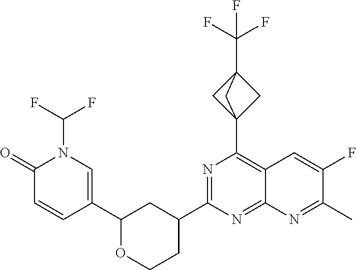 [SMILES: Cc1nc2nc(C3CCOC(c4ccc(=O)n(C(F)F)c4)C3)nc(C34CC(C(F)(F)F)(C3)C4)c2cc1F] |
| 311 | 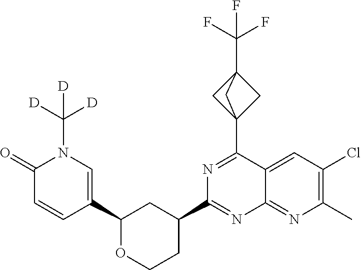 [SMILES: [2H]C([2H])([2H])n1cc([C@H]2C[C@@H](c3nc(C45CC(C(F)(F)F)(C4)C5)c4cc(Cl)c(C)nc4n3)CCO2)ccc1=O] |
| 312 | 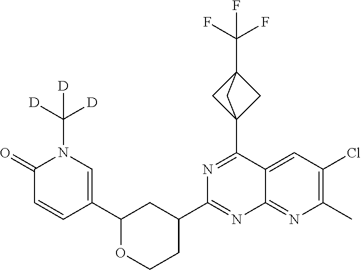 [SMILES: [2H]C([2H])([2H])n1cc(C2CC(c3nc(C45CC(C(F)(F)F)(C4)C5)c4cc(Cl)c(C)nc4n3)CCO2)ccc1=O] |
| 313 | 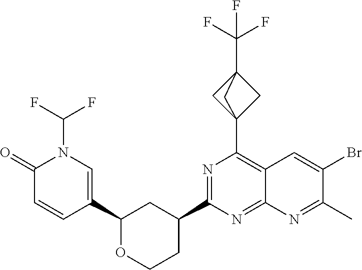 [SMILES: Cc1nc2nc([C@H]3CCO[C@@H](c4ccc(=O)n(C(F)F)c4)C3)nc(C34CC(C(F)(F)F)(C3)C4)c2cc1Br] |
| 314 | 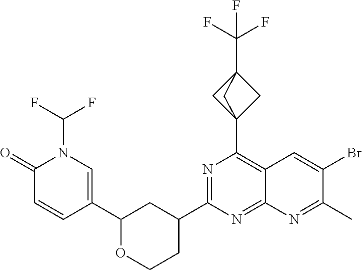 [SMILES: Cc1nc2nc(C3CCOC(c4ccc(=O)n(C(F)F)c4)C3)nc(C34CC(C(F)(F)F)(C3)C4)c2cc1Br] |
| 315 | 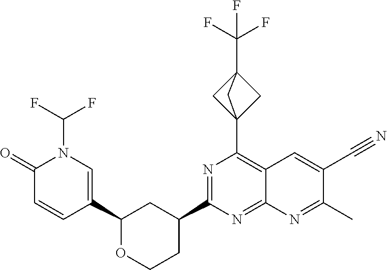 [SMILES: Cc1nc2nc([C@H]3CCO[C@@H](c4ccc(=O)n(C(F)F)c4)C3)nc(C34CC(C(F)(F)F)(C3)C4)c2cc1C#N] |
| 316 | 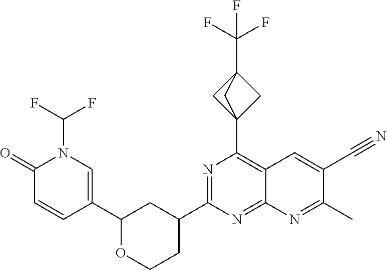 [SMILES: Cc1nc2nc(C3CCOC(c4ccc(=O)n(C(F)F)c4)C3)nc(C34CC(C(F)(F)F)(C3)C4)c2cc1C#N] |
| 317 | 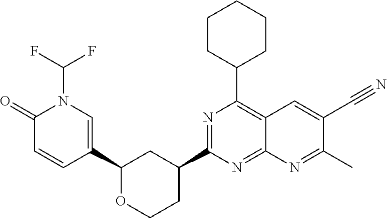 [SMILES: Cc1nc2nc([C@H]3CCO[C@@H](c4ccc(=O)n(C(F)F)c4)C3)nc(C3CCCCC3)c2cc1C#N] |
| 318 | 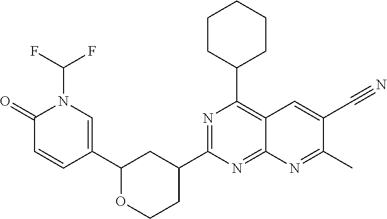 [SMILES: Cc1nc2nc(C3CCOC(c4ccc(=O)n(C(F)F)c4)C3)nc(C3CCCCC3)c2cc1C#N] |
| 319 | 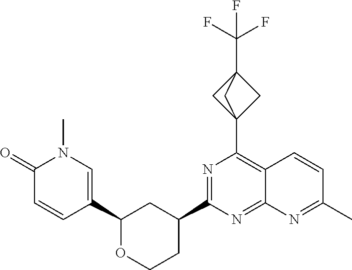 [SMILES: Cc1ccc2c(C34CC(C(F)(F)F)(C3)C4)nc([C@H]3CCO[C@@H](c4ccc(=O)n(C)c4)C3)nc2n1] |
| 320 | 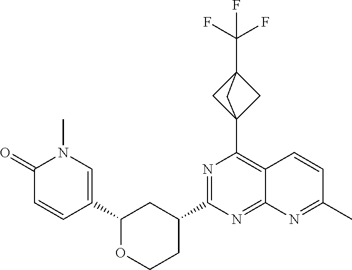 [SMILES: Cc1ccc2c(C34CC(C(F)(F)F)(C3)C4)nc([C@@H]3CCO[C@H](c4ccc(=O)n(C)c4)C3)nc2n1] |
| 321 | 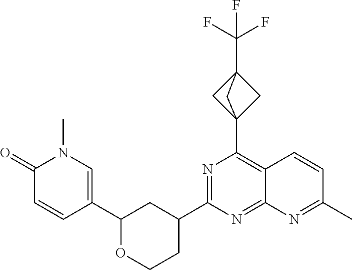 [SMILES: Cc1ccc2c(C34CC(C(F)(F)F)(C3)C4)nc(C3CCOC(c4ccc(=O)n(C)c4)C3)nc2n1] |
| 322 | 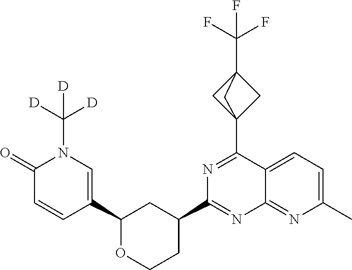 [SMILES: [2H]C([2H])([2H])n1cc([C@H]2C[C@@H](c3nc(C45CC(C(F)(F)F)(C4)C5)c4ccc(C)nc4n3)CCO2)ccc1=O] |
| 323 | 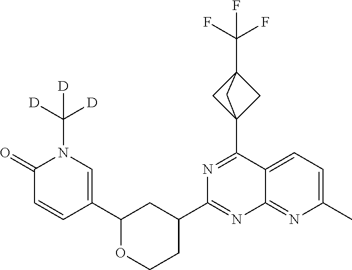 [SMILES: [2H]C([2H])([2H])n1cc(C2CC(c3nc(C45CC(C(F)(F)F)(C4)C5)c4ccc(C)nc4n3)CCO2)ccc1=O] |
| 324 | 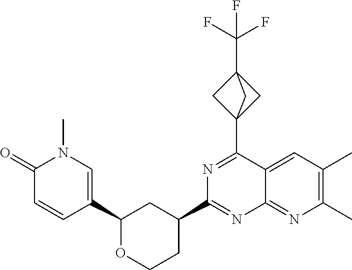 [SMILES: Cc1cc2c(C34CC(C(F)(F)F)(C3)C4)nc([C@H]3CCO[C@@H](c4ccc(=O)n(C)c4)C3)nc2nc1C] |
| 325 | 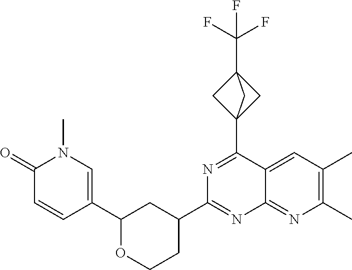 [SMILES: Cc1cc2c(C34CC(C(F)(F)F)(C3)C4)nc(C3CCOC(c4ccc(=O)n(C)c4)C3)nc2nc1C] |
| 326 | 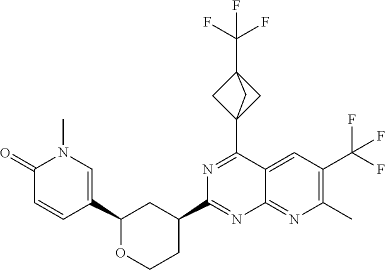 [SMILES: Cc1nc2nc([C@H]3CCO[C@@H](c4ccc(=O)n(C)c4)C3)nc(C34CC(C(F)(F)F)(C3)C4)c2cc1C(F)(F)F] |
| 327 | 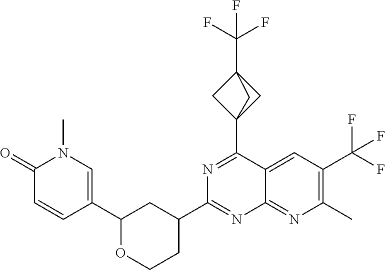 [SMILES: Cc1nc2nc(C3CCOC(c4ccc(=O)n(C)c4)C3)nc(C34CC(C(F)(F)F)(C3)C4)c2cc1C(F)(F)F] |
| 328 | 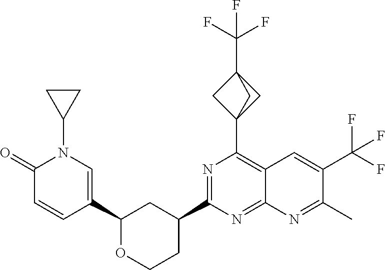 [SMILES: Cc1nc2nc([C@H]3CCO[C@@H](c4ccc(=O)n(C5CC5)c4)C3)nc(C34CC(C(F)(F)F)(C3)C4)c2cc1C(F)(F)F] |
| 329 | 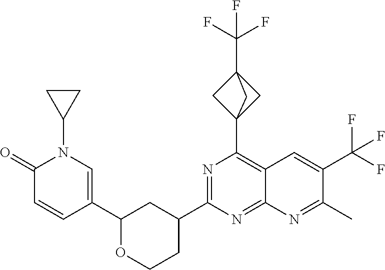 [SMILES: Cc1nc2nc(C3CCOC(c4ccc(=O)n(C5CC5)c4)C3)nc(C34CC(C(F)(F)F)(C3)C4)c2cc1C(F)(F)F] |
| 330 | 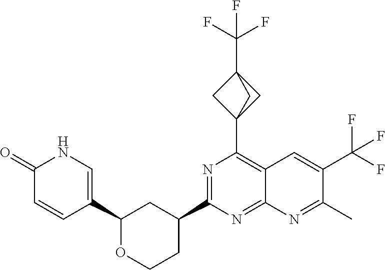 [SMILES: Cc1nc2nc([C@H]3CCO[C@@H](c4ccc(=O)[nH]c4)C3)nc(C34CC(C(F)(F)F)(C3)C4)c2cc1C(F)(F)F] |
| 331 | 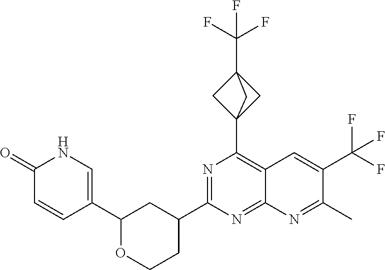 [SMILES: Cc1nc2nc(C3CCOC(c4ccc(=O)[nH]c4)C3)nc(C34CC(C(F)(F)F)(C3)C4)c2cc1C(F)(F)F] |
| 332 | 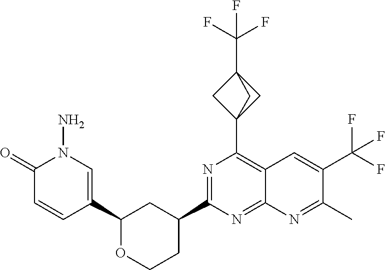 [SMILES: Cc1nc2nc([C@H]3CCO[C@@H](c4ccc(=O)n(N)c4)C3)nc(C34CC(C(F)(F)F)(C3)C4)c2cc1C(F)(F)F] |
| 333 | 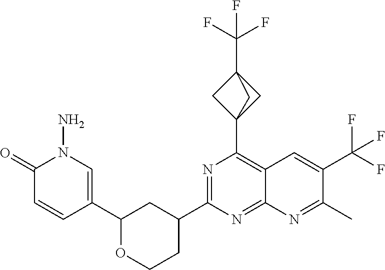 [SMILES: Cc1nc2nc(C3CCOC(c4ccc(=O)n(N)c4)C3)nc(C34CC(C(F)(F)F)(C3)C4)c2cc1C(F)(F)F] |
| 334 | 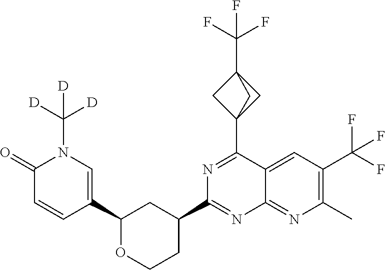 [SMILES: [2H]C([2H])([2H])n1cc([C@H]2C[C@@H](c3nc(C45CC(C(F)(F)F)(C4)C5)c4cc(C(F)(F)F)c(C)nc4n3)CCO2)ccc1=O] |
| 335 | 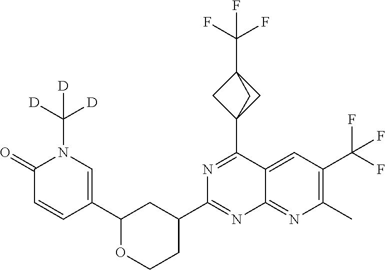 [SMILES: [2H]C([2H])([2H])n1cc(C2CC(c3nc(C45CC(C(F)(F)F)(C4)C5)c4cc(C(F)(F)F)c(C)nc4n3)CCO2)ccc1=O] |
| 336 | 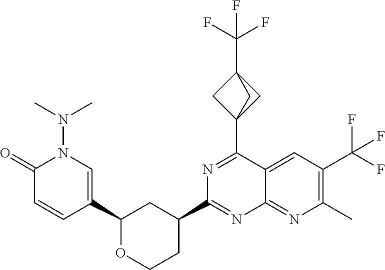 [SMILES: Cc1nc2nc([C@H]3CCO[C@@H](c4ccc(=O)n(N(C)C)c4)C3)nc(C34CC(C(F)(F)F)(C3)C4)c2cc1C(F)(F)F] |
| 337 | 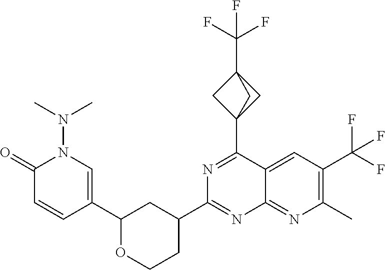 [SMILES: Cc1nc2nc(C3CCOC(c4ccc(=O)n(N(C)C)c4)C3)nc(C34CC(C(F)(F)F)(C3)C4)c2cc1C(F)(F)F] |
| 338 | 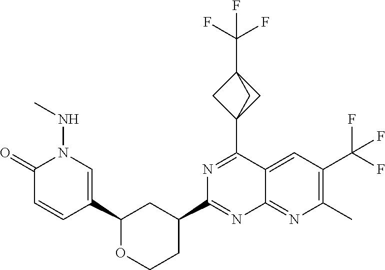 [SMILES: CNn1cc([C@H]2C[C@@H](c3nc(C45CC(C(F)(F)F)(C4)C5)c4cc(C(F)(F)F)c(C)nc4n3)CCO2)ccc1=O] |
| 339 | 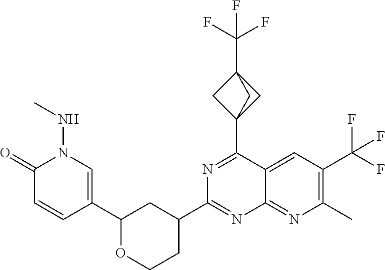 [SMILES: CNn1cc(C2CC(c3nc(C45CC(C(F)(F)F)(C4)C5)c4cc(C(F)(F)F)c(C)nc4n3)CCO2)ccc1=O] |
| 340 | 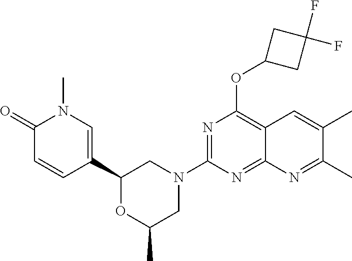 [SMILES: Cc1cc2c(OC3CC(F)(F)C3)nc(N3C[C@@H](C)O[C@@H](c4ccc(=O)n(C)c4)C3)nc2nc1C] |
| 341 | 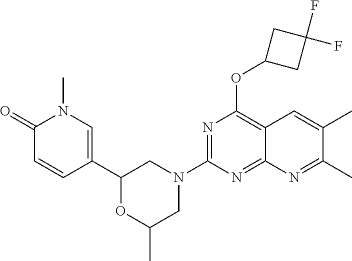 [SMILES: Cc1cc2c(OC3CC(F)(F)C3)nc(N3CC(C)OC(c4ccc(=O)n(C)c4)C3)nc2nc1C] |
| 342 | 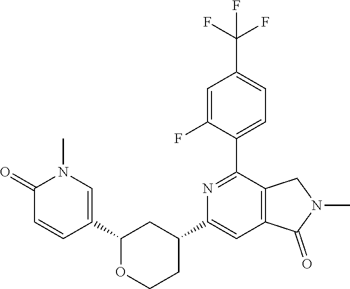 [SMILES: CN1Cc2c(cc([C@@H]3CCO[C@H](c4ccc(=O)n(C)c4)C3)nc2-c2ccc(C(F)(F)F)cc2F)C1=O] |
| 343 | 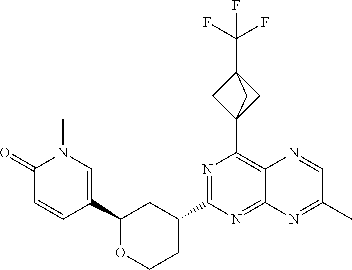 [SMILES: Cc1cnc2c(C34CC(C(F)(F)F)(C3)C4)nc([C@@H]3CCO[C@@H](c4ccc(=O)n(C)c4)C3)nc2n1] |
| 344 | 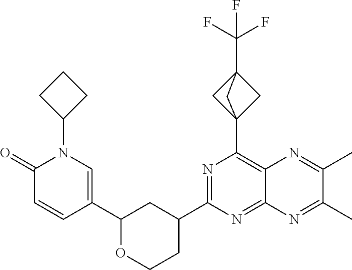 [SMILES: Cc1nc2nc(C3CCOC(c4ccc(=O)n(C5CCC5)c4)C3)nc(C34CC(C(F)(F)F)(C3)C4)c2nc1C] |
| 345 | 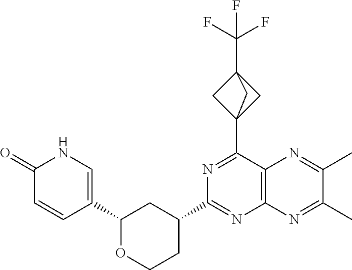 [SMILES: Cc1nc2nc([C@@H]3CCO[C@H](c4ccc(=O)[nH]c4)C3)nc(C34CC(C(F)(F)F)(C3)C4)c2nc1C] |
| 346 | 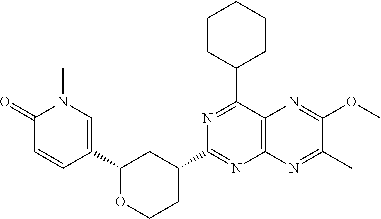 [SMILES: COc1nc2c(C3CCCCC3)nc([C@@H]3CCO[C@H](c4ccc(=O)n(C)c4)C3)nc2nc1C] |
| 347 | 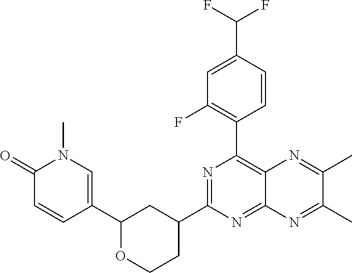 [SMILES: Cc1nc2nc(C3CCOC(c4ccc(=O)n(C)c4)C3)nc(-c3ccc(C(F)F)cc3F)c2nc1C] |
| 348 | 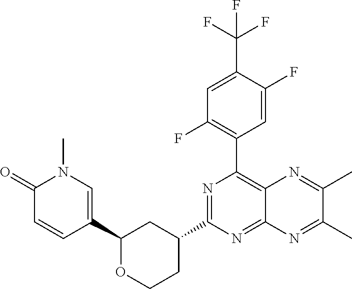 [SMILES: Cc1nc2nc([C@@H]3CCO[C@@H](c4ccc(=O)n(C)c4)C3)nc(-c3cc(F)c(C(F)(F)F)cc3F)c2nc1C] |
| 349 | 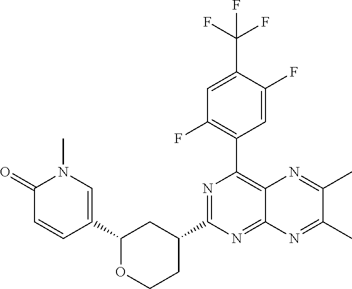 [SMILES: Cc1nc2nc([C@@H]3CCO[C@H](c4ccc(=O)n(C)c4)C3)nc(-c3cc(F)c(C(F)(F)F)cc3F)c2nc1C] |
| 350 | 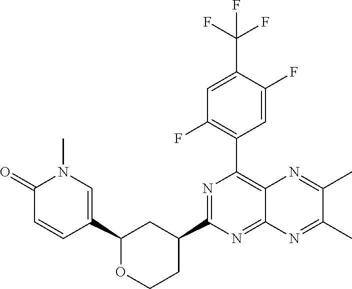 [SMILES: Cc1nc2nc([C@H]3CCO[C@@H](c4ccc(=O)n(C)c4)C3)nc(-c3cc(F)c(C(F)(F)F)cc3F)c2nc1C] |
| 351 | 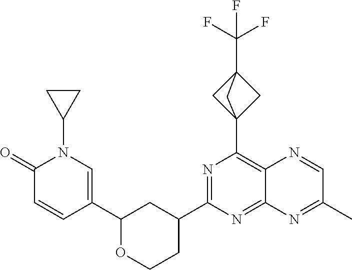 [SMILES: Cc1cnc2c(C34CC(C(F)(F)F)(C3)C4)nc(C3CCOC(c4ccc(=O)n(C5CC5)c4)C3)nc2n1] |
| 352 | 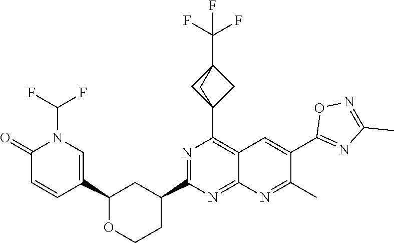 [SMILES: Cc1noc(-c2cc3c(C45CC(C(F)(F)F)(C4)C5)nc([C@H]4CCO[C@@H](c5ccc(=O)n(C(F)F)c5)C4)nc3nc2C)n1] |
| 353 | 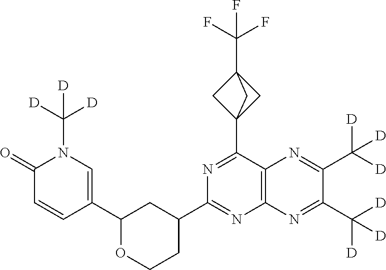 [SMILES: [2H]C([2H])([2H])c1nc2nc(C3CCOC(c4ccc(=O)n(C([2H])([2H])[2H])c4)C3)nc(C34CC(C(F)(F)F)(C3)C4)c2nc1C([2H])([2H])[2H]] |
| 354 | 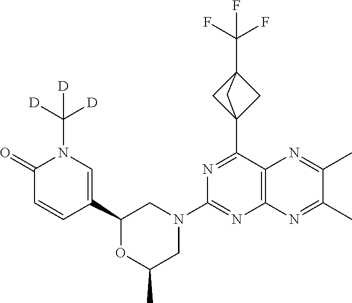 [SMILES: [2H]C([2H])([2H])n1cc([C@H]2CN(c3nc(C45CC(C(F)(F)F)(C4)C5)c4nc(C)c(C)nc4n3)C[C@@H](C)O2)ccc1=O] |
| 355 | 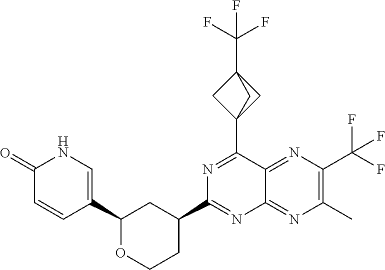 [SMILES: Cc1nc2nc([C@H]3CCO[C@@H](c4ccc(=O)[nH]c4)C3)nc(C34CC(C(F)(F)F)(C3)C4)c2nc1C(F)(F)F] |

The foregoing merely summarizes certain aspects of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

In one aspect, the disclosure provides a pharmaceutical composition comprising a compound of Formula (I), a pharmaceutically acceptable salt, solvate, stereoisomer, or tautomer thereof, or a pharmaceutically acceptable salt and/or solvate of said compound, stereoisomer, or tautomer, and a pharmaceutically acceptable excipient.

In another aspect, the disclosure provides a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition comprising said compound, salt, solvate, stereoisomer, or tautomer therefore, for use as a medicament.

### Pharmaceutically Acceptable Compositions

According to some embodiments, the present disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably activate a TREM2 protein, or a mutant thereof, in a biological sample or in a patient. In certain embodiments, the amount of compound in compositions of this disclosure is such that it is effective to measurably activate a TREM2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

Without wishing to be bound by any particular theory, the following is noted: TREM2 has been implicated in several myeloid cell processes, including phagocytosis, proliferation, survival, and regulation of inflammatory cytokine production. Ulrich and Holtzman 2016. In the last few years, TREM2 has been linked to several diseases. For instance, mutations in both TREM2 and DAP12 have been linked to the autosomal recessive disorder Nasu-Hakola Disease, which is characterized by bone cysts, muscle wasting and demyelination phenotypes. Guerreiro et al. 2013. More recently, variants in the TREM2 gene have been linked to increased risk for Alzheimer's disease (AD) and other forms of dementia including frontotemporal dementia. Jonsson et al. 2013, Guerreiro, Lohmann et al. 2013, and Jay, Miller et al. 2015. In particular, the R47H variant has been identified in genome-wide studies as being associated with increased risk for late-onset AD with an overall adjusted odds ratio (for populations of all ages) of 2.3, second only to the strong genetic association of ApoE to Alzheimer's. The R⁴⁷H mutation resides on the extracellular lg V-set domain of the TREM2 protein and has been shown to impact lipid binding and uptake of apoptotic cells and Abeta (Wang et al. 2015; Yeh et al. 2016), suggestive of a loss-of-function linked to disease. Further, postmortem comparison of AD patients' brains with and without the R⁴⁷H mutation are supportive of a novel loss-of-microglial barrier function for the carriers of the mutation, with the R⁴⁷H carrier microglia putatively demonstrating a reduced ability to compact plaques and limit their spread. Yuan et al. 2016. Impairment in microgliosis has been reported in animal models of prion disease, multiple sclerosis, and stroke, suggesting that TREM2 may play an important role in supporting microgliosis in response to pathology or damage in the central nervous system. Ulrich and Holtzman 2016. In addition, knockdown of TREM2 has been shown to aggravate a-syn-induced inflammatory responses in vitro and exacerbate dopaminergic neuron loss in response to AAV-SYN in vivo (a model of Parkinson's disease), suggesting that impaired microglial TREM2 signaling exacerbates neurodegeneration by modulating microglial activation states. Guo et. al. 2019. A variety of animal models also suggest that Toll-Like Receptor (TLR) signaling is important in the pathogenesis of Rheumatoid Arthritis (RA) via persistent expression of pro-inflammatory cytokines by macrophages. Signaling through TREM2/DAP12 inhibits TLR responses by reducing MAPK (Erk1/2) activation, suggesting that TREM2 activation may act as a negative regulator of TLR driven R^{A} pathogenesis. Huang and Pope 2009.

In view of the data indicating that deficits in TREM2 activity affect macrophage and microglia function, the compounds disclosed herein are of particular use in disorders, such as those described above and in the embodiments that follow and in neurodegenerative disorders more generally.

In one aspect, the disclosure provides a compound of Formula (I), or a tautomer thereof, or a pharmaceutically acceptable salt of said compound or said tautomer, or a pharmaceutical composition thereof for use in treating or preventing a condition associated with a loss of function of human TREM2.

In another aspect, the present disclosure features a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in treating a disease, disorder, or condition. Exemplary diseases, disorders, or conditions include proliferative diseases, cardiovascular diseases, metabolic diseases, inflammatory diseases, autoimmune disorders, neurodegenerative disorders, infectious diseases, and tissues injuries.

In some embodiments, the proliferative disease is a benign condition, e.g., a benign neoplasm. In some embodiments, the proliferative disease is a cancer. The cancer may be a cancer of any cell or tissue in the body, for example, a cancer of the brain, eye, thyroid, breast, lung, stomach, kidney, pancreas, bladder, colon, rectum, uterus, ovaries, prostate, skin, fibrous tissues, lympathic system, bone marrow, blood, or immune system. The cancer may comprise a tumor or solid cancer (e.g., a carcinoma) or a non-mass cancer. Exemplary cancers include glioblastoma, retinoblastoma, skin cancer, ocular cancer, gastrointestinal cancer, breast cancer, lung cancer, ductal carcinoma, lungadenocarcinoma, lymphoma, endometrial cancer, liver cancer, pancreatic cancer, renal cell cancer, ovarian cancer, fibrosarcoma, leukemia, myeloma, and polycythemia vera.

In some embodiments, the disease, disorder, or condition is cardiovascular. Exemplary cardiovascular diseases, disorders, and conditions include stroke, coronary heart disease, cardiomyopathy, arrhythmia (e.g. atrial fibrillation), aortic aneurysms, and venous thrombosis.

In some embodiments, the disease, disorder, or condition is metabolic disease. Exemplary metabolic diseases include diabetes (e.g., Type 1 diabetes or Type 2 diabetes), metabolic dysfunction-associated steatohepatitis (MASH), non-alcoholic steatohepatitis (NASH), and Gaucher's disease.

In some embodiments, the disease, disorder, or condition is an inflammatory disease. Exemplary inflammatory diseases include arthritis, acute and chronic colitis, ulcerative colitis, inflammatory bowel disease, Behcet's disease, and granulomatous disorders.

In some embodiments, the disease, disorder, or condition is an autoimmune disease. Exemplary autoimmune diseases include diabetes (e.g., Type 1 diabetes), lupus, sarcoidosis, and multiple sclerosis.

In some embodiments, the disease, disorder, or condition is a neurological disease. In some embodiments, the neurological disease is a neurodegenerative disease. Exemplary neurodegernative diseases include dementia, Alzheimer's disease, Creutzfeldt-Jakob disease, Parkinson's disease, demential with Lewy bodies, amyotrophic lateral sclerosis (ALS), Huntington's disease, taupathy disease, Nasu-Hakola disease, dry age-related macular degeneration (dry AMD), multiple system atrophy (MSA), Shy-Drager syndrome, progressive supranuclear palsy, cortical basal ganglionic degeneration, glaucoma, retinitis pigmentosa, and retinal degeneration, In other embodiments, the neurological condition is acute trauma, chronic trauma, acute disseminated encephalomyelitis, cognitive deficit and memory loss, essential tremor, central nervous system (CNS) lupus, normal pressure hydrocephalus, and seizures.

In some embodiments, the disease, disorder, or condition is an infectious disease. An infectious disease may be local or systemic. Exemplary infectious diseases include eye infections, malaria, respiratory tract infections, sepsis, herpes (e.g., CNS herpes), parasitic infections, Trypanosome infection, Cruzi infection, Pseudomonas aeruginosa infection, Leishmania donovani infection, group B Streptococcus infection, Campylobacter jejuni infection, Neisseria meningitidis infection, type I HIV, and Haemophilus influenza.

In some embodiments, the disease, disorder, or condition is a tissue injury. Any tissues of the body may be injured. Exemplary injuries include ocular (e.g. hyphoma), spinal cord injury, traumatic brain injury, hepatocellular, and wound repair in diabetes.

In one aspect, the disclosure provides a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for treating or preventing a disease, disorder or condition. In one aspect, the disclosure provides a compound of Formula (I), or a tautomer thereof, or a pharmaceutically acceptable salt of said compound or said tautomer, or a pharmaceutical composition thereof for use in treating or preventing Parkinson's disease, rheumatoid arthritis, Alzheimer's disease, Nasu-Hakola disease, frontotemporal dementia, multiple sclerosis, prion disease, or stroke.

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing a condition associated with a loss of function of human TREM2.

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing Parkinson's disease, rheumatoid arthritis, Alzheimer's disease, Nasu-Hakola disease, frontotemporal dementia, multiple sclerosis, prion disease, or stroke.

In another aspect, the disclosure provides a method of treating or preventing a condition associated with a loss of function of human TREM2 in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

In another aspect, the disclosure provides a method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

### CSF1R

CSF1R is a cell-surface receptor primarily for the cytokine colony stimulating factor 1 (CSF-1), also known until recently as macrophage colony-stimulating factor (M-CSF), which regulates the survival, proliferation, differentiation and function of mononuclear phagocytic cells, including microglia of the central nervous system. CSF1R is composed of a highly glycosylated extracellular ligand-binding domain, a trans-membrane domain and an intracellular tyrosine-kinase domain. Binding of CSF-1 to CSF1R results in the formation of receptor homodimers and subsequent auto-phosphorylation of several tyrosine residues in the cytoplasmic domain, notably Syk. In the brain, CSF1R is predominantly expressed in microglial cells. It has been found that microglia in CSF1R+/− patients are depleted and show increased apoptosis (Oosterhof et al., 2018).

The present disclosure relates to the unexpected discovery that administration of a TREM2 agonist can rescue the loss of microglia in cells having mutations in CSF1R. It has been previously shown that TREM2 agonist antibody 4D9 increases ATP luminescence (a measure of cell number and activity) in a dose dependent manner when the levels of M-CSF in media are reduced to 5 ng/ml (Schlepckow et al, EMBO Mol Med., 2020) and that TREM2 agonist AL002c increases ATP luminescence when M-CSF is completely removed from the media (Wang et al, J. Exp. Med.; 2020, 217 (9): e20200785). This finding suggests that TREM2 agonism can compensate for deficiency in CSF1R signaling caused by a decrease in the concentration of its ligand. In a 5×FAD murine Alzheimer's disease model of amyloid pathology, doses of a CSF1R inhibitor that almost completely eliminate microglia in the brains of wild-type animals show surviving microglia clustered around the amyloid plaques (Spangenberg et al, Nature Communications 2019). Plaque amyloid has been demonstrated in the past to be a ligand for TREM2, and it has been shown that microglial engagement with amyloid is dependent on TREM2 (Condello et al, Nat Comm., 2015). The present disclosure relates to the unexpected discovery that it is activation of TREM2 that rescued the microglia in the presence of the CSF1R inhibitor, and that this effect is also observed in patients suffering from loss of microglia due to CSF1R mutation. This discovery has not been previously taught or suggested in the available art.

To date, no prior study has shown that TREM2 agonism can rescue the loss of microglia in cells where mutations in the CSF1R kinase domain reduce CSF1R activity, rather than the presence of a CSF1R inhibitor or a deficiency in CSF1R ligand. Furthermore, no prior study has taught or suggested that reversal of the loss of microglia due to a CSF1R mutation through TREM2 agonism can be used to treat a disease or disorder caused by and/or associated with a CSF1R mutation.

Adult-onset leukoencephalopathy with axonal spheroids and pigmented glia (ALSP), previously recognized as hereditary diffuse leukoencephalopathy with axonal spheroids (HDLS) or pigmentary orthochromatic leukodystrophy (POLD), is an autosomal-dominant central nervous system disease that manifests in the form of variable behavioral, cognitive and motor function changes in patients suffering from the disease. ALSP is characterized by patchy cerebral white matter abnormalities visible by magnetic resonance imaging. However, the clinical symptoms and MRI changes are not specific to ALSP and are common for other neurological conditions, including Nasu-Hakola disease (NHD) and AD, making diagnosis and treatment of ALSP very difficult.

Recent studies have discovered that ALSP is a Mendelian disorder in which patients carry a heterozygous loss of function mutation in the kinase domain of CSF1R, suggesting a reduced level of signaling on the macrophage colony-stimulating factor (M-CSF)/CSF1R axis (Rademakers et al, Nat Genet 2012; Konno et al, Neurology 2018). In one aspect, the present disclosure relates to the surprising discovery that activation of the TREM2 pathway can rescue the loss of microglia in CSF1R+/−ALSP patients, preventing microglia apoptosis, thereby treating the ALSP condition.

In one aspect, the disclosure provides a compound of Formula (I), or a tautomer thereof, or a pharmaceutically acceptable salt of said compound or said tautomer, or a pharmaceutical composition thereof for use in treating or preventing a condition associated with dysfunction of Colony stimulating factor 1 receptor (CSF1R, also known as macrophage colony-stimulating factor receptor/M-CSFR, or cluster of differentiation 115/CD₁₁₅).

In one aspect, the disclosure provides a compound of Formula (I), or a tautomer thereof, or a pharmaceutically acceptable salt of said compound or said tautomer, or a pharmaceutical composition thereof for use in treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing a condition associated with dysfunction of CSF1R.

In one aspect, the disclosure provides a compound of Formula (I), or a tautomer thereof, or a pharmaceutically acceptable salt of said compound or said tautomer, or a pharmaceutical composition thereof for use in the preparation of a medicament for treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In another aspect, the disclosure provides a method of treating or preventing a disease or disorder associated with dysfunction of CSF1R in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the subject is selected for treatment based on a diagnosis that includes the presence of a mutation in a CSF1R gene affecting the function of CSF1R. In some embodiments, the mutation in the CSF1R gene is a mutation that causes a decrease in CSF1R activity or a cessation of CSF1R activity. In some embodiments, the disease or disorder is caused by a heterozygous CSF1R mutation. In some embodiments, the disease or disorder is caused by a homozygous CSF1R mutation. In some embodiments, the disease or disorder is caused by a splice mutation in the csf1r gene. In some embodiments, the disease or disorder is caused by a missense mutation in the csf1r gene. In some embodiments, the disease or disorder is caused by a mutation in the catalytic kinase domain of CSF1R. In some embodiments, the disease or disorder is caused by a mutation in an immunoglobulin domain of CSF1R. In some embodiments, the disease or disorder is caused by a mutation in the ectodomain of CSF1R. In some embodiments, the disease or disorder is a disease or disorder resulting from a change (e.g. increase, decrease or cessation) in the activity of CSF1R. In some embodiments, the disease or disorder is a disease or disorder resulting from a decrease or cessation in the activity of CSF1R. CSF1R related activities that are changed in the disease or disorder include, but are not limited to: decrease or loss of microglia function; increased microglia apoptosis; decrease in Src signaling; decrease in Syk signaling; decreased microglial proliferation; decreased microglial response to cellular debris; decreased phagocytosis; and decreased release of cytokines in response to stimuli. In some embodiments, the disease or disorder is caused by a loss-of-function mutation in CSF1R. In some embodiments, the loss-of-function mutation results in a complete cessation of CSF1R function. In some embodiments, the loss-of-function mutation results in a partial loss of CSF1R function, or a decrease in CSF1R activity.

In another aspect, the disclosure provides a method of treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats or prevents ALSP, which is an encompassing and superseding name for both HDLS and POLD. In some embodiments, the disease or disorder is a homozygous mutation in CSF1R. In some embodiments, the method treats or prevents pediatric-onset leukoencephalopathy. In some embodiments, the method treats or prevents congenital absence of microglia. In some embodiments, the method treats or prevents brain abnormalities neurodegeneration and dysosteosclerosis (BANDDOS).

In yet another aspect, the disclosure provides a method of treating or preventing Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting CSF1R dysfunction, or having a mutation in a gene affecting the function of CSF1R, the method comprising administering to the subject a therapeutically effective amount of a compound of the present disclosure, or a tautomer thereof, or a pharmaceutically acceptable salt of said compound or said tautomer, or a pharmaceutical composition thereof.

### ABCD1

The ABCD1 gene provides instructions for producing the adrenoleukodystrophy protein (ALDP). ABCD1 (ALDP) maps to Xq28. ABCD1 is a member of the ATP-binding cassette (ABC) transporter superfamily. The superfamily contains membrane proteins that translocate a wide variety of substrates across extra- and intracellular membranes, including metabolic products, lipids and sterols, and drugs. ALDP is located in the membranes of cell structures called peroxisomes. Peroxisomes are small sacs within cells that process many types of molecules. ALDP brings a group of fats called very long-chain fatty acids (VLCFAs) into peroxisomes, where they are broken down. As ABCD1 is highly expressed in microglia, it is possible that microglial dysfunction and their close interaction with other cell types actively participates in neurodegenerative processes (Gong et al., Annals of Neurology. 2017; 82 (5): 813-827.). It has been shown that severe microglia loss and damage is an early feature in patients with cerebral form of x-linked ALD (cALD) carrying ABCD1 mutations (Bergner et al., Glia. 2019; 67:1196-1209). It has also been shown that ABCD1-deficiency leads to an impaired plasticity of myeloid lineage cells that is reflected in incomplete establishment of anti-inflammatory responses, thus possibly contributing to the devastating rapidly progressive demyelination in cerebral adrenoleukodystrophy (Weinhor et al., BRAIN 2018:141; 2329-2342). These findings emphasize microglia/monocytes/macrophages as crucial therapeutic targets for preventing or stopping myelin destruction in patients with X-linked adrenoleukodystrophy.

The present disclosure relates to the unexpected discovery that administration of a TREM2 agonist can rescue the loss of microglia in cells having mutations in the ABCD1 gene. It has been previously shown that TREM2 agonist antibody 4D9 increases ATP luminescence (a measure of cell number and activity) in a dose dependent manner when the levels of M-CSF in media are reduced to 5 ng/ml (Schlepckow et al, EMBO Mol Med., 2020) and that TREM2 agonist AL002c increases ATP luminescence when M-CSF is completely removed from the media (Wang et al, J. Exp. Med.; 2020, 217 (9): e20200785). This finding suggests that TREM2 agonism can compensate for deficiency in ABCD1 function leading to sustained activation, proliferation, chemotaxis of microglia, maintenance of anti-inflammatory environment and reduced astrocytosis caused by a decrease in ABCD1 and accumulation of VLCFAs. The present disclosure relates to the unexpected discovery that activation of TREM2 can rescue the microglia in the presence of the ABCD1 mutation and an increase in VLCFA, and that this effect may be also observed in patients suffering from loss of microglia due to ABCD1 mutation. This discovery has not been previously taught or suggested in the available art.

To date, no prior study has shown that TREM2 agonism can rescue the loss of microglia in cells where mutations in the ABCD1 and a VLCFA increase is present. No prior study has taught or suggested that reversal of the loss of microglia due to an ABCD1 mutation through TREM2 agonism can be used to treat a disease or disorder caused by and/or associated with an ABCD1 mutation.

In one aspect, the disclosure provides a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in treating or preventing a condition associated with dysfunction of ATP-binding cassette transporter 1 (ABCD1).

In one aspect, the disclosure provides a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In one aspect, the disclosure provides a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in the preparation of a medicament for treating or preventing a condition associated with dysfunction of ABCD1.

In one aspect, the invention provides a compound of Formula (I), a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in the preparation of a medicament for treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In yet another aspect, the disclosure provides a method of treating or preventing a disease or disorder associated with dysfunction of ABCD1 in a subject in need thereof, the method comprising administering to the subject a therapeutically effective amount of a compound of the present disclosure, a pharmaceutically acceptable salt, solvate, stereoisomer, or tautomer thereof, or a pharmaceutically acceptable salt and/or solvate of said compound, stereoisomer, or tautomer, or a pharmaceutical composition thereof. In some embodiments, the patient is selected for treatment based on a diagnosis that includes the presence of a mutation in an ABCD1 gene affecting the function of ABCD1. In some embodiments, the mutation in the ABCD1 gene is a mutation that causes a decrease in ABCD1 activity or a cessation of ABCD1 activity. In some embodiments, the disease or disorder is caused by a heterozygous ABCD1 mutation. In some embodiments, the disease or disorder is caused by a homozygous ABCD1 mutation. In some embodiments, the disease or disorder is caused by a splice mutation in the ABCD1 gene. In some embodiments, the disease or disorder is caused by a missense mutation in the ABCD1 gene. In some embodiments, the disease or disorder is a disease or disorder resulting from a change (e.g. increase, decrease or cessation) in the activity of ABCD1. In some embodiments, the disease or disorder is a disease or disorder resulting from a decrease or cessation in the activity of ABCD1. ABCD1 related activities that are changed in the disease or disorder include, but are not limited to peroxisomal import of fatty acids and/or fatty acyl-CoAs and production of adrenoleukodystrophy protein (ALDP). In some embodiments, the disease or disorder is caused by a loss-of-function mutation in ABCD1. In some embodiments, the loss-of-function mutation results in a complete cessation of ABCD1 function. In some embodiments, the loss-of-function mutation results in a partial loss of ABCD1 function, or a decrease in ABCD1 activity. In some embodiments, the disease or disorder is caused by a homozygous mutation in ABCD1. In some embodiments, the disease or disorder is a neurodegenerative disorder. In some embodiments, the disease or disorder is a neurodegenerative disorder caused by and/or associated with an ABCD1 dysfunction. In some embodiments, the disease or disorder is an immunological disorder. In some embodiments, the disease or disorder is an immunological disorder caused by and/or associated with an ABCD1 dysfunction.

In yet another aspect, the disclosure provides a method of treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX) in a subject in need thereof, the method comprising administering to the subject a therapeutically effective amount of a compound of the present disclosure, a pharmaceutically acceptable salt, solvate, stereoisomer, or tautomer thereof, or a pharmaceutically acceptable salt and/or solvate of said compound, stereoisomer, or tautomer, or a pharmaceutical composition thereof. In some embodiments, any of the aforementioned diseases are present in a patient exhibiting ABCD1 dysfunction or having a mutation in a gene affecting the function of ABCD1. In some embodiments, the method treats or prevents X-linked adrenoleukodystrophy (x-ALD). In some embodiments, the x-ALD is a cerebral form of x-linked ALD (cALD). In some embodiments, the method treats or prevents Addison disease wherein the patient has been found to have a mutation in one or more ABCD1 genes affecting ABCD1 function. In some embodiments, the method treats or prevents Addison disease, wherein the patient has a loss-of-function mutation in ABCD1.

In yet another aspect, the disclosure provides a method of treating or preventing Nasu-Hakola disease, Alzheimer's disease, frontotemporal dementia, multiple sclerosis, Guillain-Barre syndrome, amyotrophic lateral sclerosis (ALS), or Parkinson's disease, wherein any of the aforementioned diseases or disorders are present in a patient exhibiting ABCD1 dysfunction, or having a mutation in a gene affecting the function of ABCD1, the method comprising administering to the subject a therapeutically effective amount of a compound of the present disclosure, a pharmaceutically acceptable salt, solvate, stereoisomer, or tautomer thereof, or a pharmaceutically acceptable salt and/or solvate of said compound, stereoisomer, or tautomer, or a pharmaceutical composition thereof.

### Autism Spectrum Disorders

It has been found that TREM2 deficient mice exhibit symptoms reminiscent of autism spectrum disorders (ASDs)(Filipello et al., Immunity, 2018, 48, 979-991). It has also been found that microglia depletion of the autophagy Aatg7 gene results in defective synaptic pruning and results in increased dendritic spine density, and abnormal social interaction and repetitive behaviors indicative of ASDs (Kim, et al., Molecular Psychiatry, 2017, 22, 1576-1584.). Further studies have shown that increased dendritic spin density detected in post-mortem ASD brains, likely caused by defective synaptic pruning, results in circuit hypoconnectivity and behavioral defects and are a potential origin of a number of neurodevelopmental diseases (Tang, et al., Neuron, 2014, 83, 1131-1143). Without intending to be limited to any particular theory, these findings suggest that TREM2 activation can reverse microglia depletion, and therefore correct the defective synaptic pruning that is central to neurodevelopmental diseases such as ASDs. The present disclosure relates to the unexpected discovery that activation of TREM2, using a compound of the present disclosure, can rescue microglia in subjects suffering from an ASD. This discovery has not been previously taught or suggested in the available art.

In another aspect, the present invention provides a compound of the present disclosure, a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in treating autism or autism spectrum disorders.

In yet another aspect, the present invention provides a compound of the present disclosure, a pharmaceutically acceptable salt, solvate, stereoisomer, or tautomer thereof, or a pharmaceutically acceptable salt and/or solvate of said compound, stereoisomer, or tautomer, or a pharmaceutical composition thereof for use in the preparation of a medicament for treating autism or autism spectrum disorders.

In yet another aspect, the present invention provides a method of treating autism or autism spectrum disorders in a subject in need thereof, the method comprising administering to the subject a therapeutically effective amount of a compound of the present disclosure, a pharmaceutically acceptable salt, solvate, stereoisomer, or tautomer thereof, or a pharmaceutically acceptable salt and/or solvate of said compound, stereoisomer, or tautomer, or a pharmaceutical composition thereof. In some embodiments, the method treats autism. In some embodiments, the method treats Asperger syndrome.

In some embodiments, the disclosure provides a method of increasing the activity of TREM2, the method comprising contacting a compound of the present disclosure, a pharmaceutically acceptable salt, solvate, stereoisomer, or tautomer thereof, or a pharmaceutically acceptable salt and/or solvate of said compound, stereoisomer, or tautomer with the TREM2. In some embodiments, the contacting takes place in vitro. In some embodiments, the contacting takes place in vivo. In some embodiments, the TREM2 is human TREM2.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein, a pharmaceutically acceptable salt, solvate, stereoisomer, or tautomer thereof, or a pharmaceutically acceptable salt and/or solvate of said compound, stereoisomer, or tautomer and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this disclosure may also be combined with include, without limitation: treatments for Parkinson's disease, rheumatoid arthritis, Alzheimer's disease, Nasu-Hakola disease, frontotemporal dementia, multiple sclerosis, prion disease, or stroke.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the present disclosure are administered as a multiple dosage regimen within greater than 24 hours a parts.

In one embodiment, the present disclosure provides a composition comprising a provided compound, a pharmaceutically acceptable salt, solvate, stereoisomer, or tautomer thereof, or a pharmaceutically acceptable salt and/or solvate of said compound, stereoisomer, or tautomer and one or more additional therapeutic agents. The therapeutic agent may be administered together with a provided compound a pharmaceutically acceptable salt, solvate, stereoisomer, or tautomer thereof, or a pharmaceutically acceptable salt and/or solvate of said compound, stereoisomer, or tautomer, or may be administered prior to or following administration of a provided compound, a pharmaceutically acceptable salt, solvate, stereoisomer, or tautomer thereof, or a pharmaceutically acceptable salt and/or solvate of said compound, stereoisomer, or tautomer. Suitable therapeutic agents are described in further detail below. In certain embodiments, a provided compound, a pharmaceutically acceptable salt, solvate, stereoisomer, or tautomer thereof, or a pharmaceutically acceptable salt and/or solvate of said compound, stereoisomer, or tautomer may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound, a pharmaceutically acceptable salt, solvate, stereoisomer, or tautomer thereof, or a pharmaceutically acceptable salt and/or solvate of said compound, stereoisomer, or tautomer may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### Definitions

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

As used herein, the following definitions shall apply unless otherwise indicated. For purposes of this disclosure, the chemical elements are identified in accordance with the Periodic Table of the Elements, CAS version, Handbook of Chemistry and Physics, 101st Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8th Ed., Ed.: Smith, M. B., John Wiley & Sons, New York: 2019, the entire contents of which are hereby incorporated by reference.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. For example, (1R)-1-methyl-2-(trifluoromethyl)cyclohexane is meant to encompass (1R,2R)-1-methyl-2-(trifluoromethyl)cyclohexane and (1R,2S)-1-methyl-2-(trifluoromethyl)cyclohexane. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. For example, the following is illustrative of tautomers of the compounds of Formula (I), wherein Ring A, together with the 6-membered ring system to which it is fused, forms a bicyclic ring system of

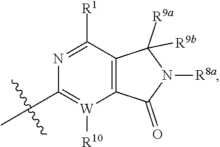

and wherein R^{8a} is H:

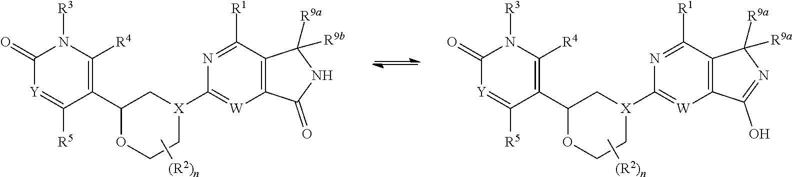

Additionally, a second tautomer is possible for compounds of Formula (I) wherein R³ is H:

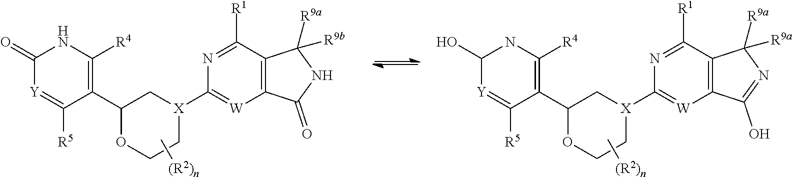

Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula (I), wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula (I), for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

The term “alkyl”, as used herein, means a straight-chain (i.e., unbranched) or branched, substituted or unsubstituted hydrocarbon chain that is completely saturated or that contains one or more units of unsaturation, that has a single point of attachment to the rest of the molecule. Unless otherwise specified, alkyl groups contain 1 to 6 alkyl carbon atoms. In some embodiments, alkyl groups contain 1 to 5 alkylcarbon atoms. In other embodiments, alkylgroups contain 1 to 4 alkylcarbon atoms. In still other embodiments, alkylgroups contain 1 to 3 alkylcarbon atoms, and in yet other embodiments, alkylgroups contain 1 to 2 alkylcarbon atoms.

The term “cycloalkyl” or “carbocyclic” as used herein, means a hydrocarbon ring, substituted or unsubstituted that is completely saturated or that contains one or more units of unsaturation but which is not aromatic, that has a single point of attachment to the rest of the molecule. In some embodiments “cycloalkyl,” refers to a monocyclic or bicyclic, bridged bicyclic, or spirocyclic ring, C₃₋₁₂ hydrocarbon that is completely saturated or that contains one or more units of unsaturation, but which is not aromatic, that has a single point of attachment to the rest of the molecule. Suitable cycloalkyl groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for alkyl groups. Additionally or alternatively, any substitutable nitrogen of a bridged bicyclic group is optionally substituted. Exemplary bicyclic rings include:

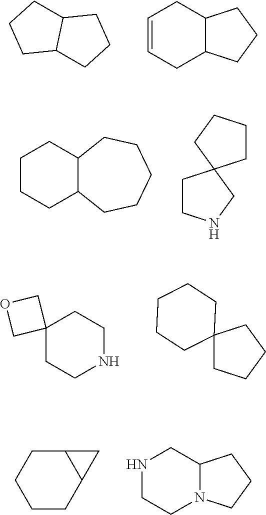

Exemplary bridged bicyclics include:

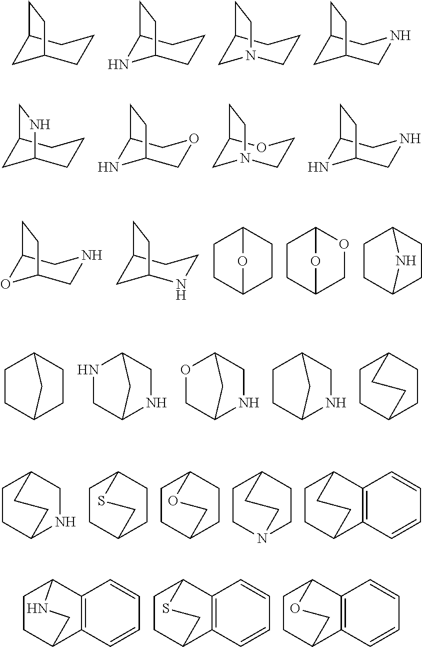

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “C₁₋₆ haloalkyl” refers to a C₁₋₆ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted alkyl group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted alkyl group.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloalkyl rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The terms “C₁₋₃alkyl,” “C₁₋₅alkyl,” and “C₁₋₆alkyl” as used herein refer to a straight or branched chain hydrocarbon containing from 1 to 3, 1 to 5, and 1 to 6 carbon atoms, respectively. Representative examples of C₁₋₃alkyl, C₁₋₅alky, or C₁₋₆alkyl include, but are not limited to, methyl, ethyl, n-propyl, iso-propyl, n-butyl, sec-butyl, iso-butyl, tert-butyl, pentyl and hexyl.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₃₋₆cycloalkyl” as used herein refers to a saturated carbocyclic molecule wherein the cyclic framework has 3 to 6 carbon atoms. Representative examples of C₃₋₅cycloalkyl include, but are not limited to, cyclopropyl, cyclobutyl, cyclopentyl, and cyclohexyl.

The terms “diC₁₋₃alkylamino” as used herein refer to-NR*R**, wherein R* and R** independently represent a C₁₋₃alkyl as defined herein. Representative examples of diC₁₋₃alkylamino include, but are not limited to, —N(CH₃)₂, —N(CH₂CH₃)₂, —N(CH₃)(CH₂CH₃), —N(CH₂CH₂CH₃)₂, and —N(CH(CH₃)₂)₂.

The term “C₁₋₃alkoxy” and “C₁₋₆alkoxy” as used herein refer to —OR^{#}, wherein R^{#} represents a C₁₋₃alkyl and C₁₋₆alkyl group, respectively, as defined herein. Representative examples of C₁₋₃alkoxy or C₁₋₆alkoxy include, but are not limited to, methoxy, ethoxy, propoxy, iso-propoxy, and butoxy. The term “halogen” as used herein refers to —F, —Cl, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₆haloalkyl” refers to a C₁₋₆alkyl as defined herein, wherein one or more hydrogen atoms are substituted with a halogen. Representative examples of C₁₋₆haloalkyl include, but are not limited to, —CH₂F, —CHF₂, —CF₃, —CHFCl, —CH₂CF₃, —CFHCF₃, —CF₂CF₃, —CH (CF₃)₂, —CF (CHF₂)₂, and —CH(CH₂F)(CF₃). Further, the term “C₁₋₆haloalkoxy” for example refers to a C₁₋₆alkoxy as defined herein, wherein one or more hydrogen atoms are substituted with a halogen. Representative examples of C₁₋₆haloalkoxy include, but are not limited to, —OCH₂F, —OCHF₂, —OCF₃, —OCHFCl, —OCH₂CF₃, —OCFHCF₃, —OCF₂CF₃, —OCH(CF₃)₂, —OCF (CHF₂)₂, and —OCH(CH₂F)(CF₃).

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆heterocycloalkyl” as used herein refers to a saturated carbocyclic molecule wherein the cyclic framework has 3 to 6 carbons and wherein one carbon atom is substituted with a heteroatom selected from N, O, and S. If the C₃₋₆heterocycloalkyl group is a C₆heterocycloalkyl, one or two carbon atoms are substituted with a heteroatom independently selected from N, O, and S. Representative examples of C₃₋₆heterocycloalkyl include, but are not limited to, aziridinyl, azetidinyl, oxetanyl, pyrrolidinyl, piperazinyl, morpholinyl, and thiomorpholinyl.

The term “C₅₋₈spiroalkyl” as used herein refers a bicyclic ring system, wherein the two rings are connected through a single common carbon atom. Representative examples of C₅₋₈spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈tricycloalkyl” as used herein refers a tricyclic ring system, wherein all three cycloalkyl rings share the same two ring atoms. Representative examples of C₅₋₈tricycloalkyl include, but are not limited to, tricyclo[1.1.1.0^{1,3}]pentanyl,

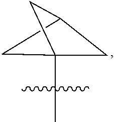 [SMILES: *C12CC(C1)C2]

tricyclo[2.1.1.0^{1,4}]hexanyl, tricyclo[3.1.1.0^{1,5}]hexanyl, and tricyclo[3.2.1.0^{1,5}]octanyl.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cyclo alkyl, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66 (1): 1-19 (1977). See also Stahl et al., Pharmaceutical Salts: Properties, Selection, and Use, 2nd Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner. As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid and gas phase), extraction, distillation, trituration, and reverse phase HPLC.

The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

### EXAMPLES

This section provides specific examples of compounds of Formula (I) and methods of making the same.

### List of Abbreviations

| | |
|---|---|
| AcOH | acetic acid |
| Amphos | di-tert-butyl(4-dimethylaminophenyl)phosphine |
| aq or aq. | aqueous |
| bipy | 2,2′-bipyridine |
| Bn | benzyl |
| CAN | ceric ammonium nitrate |
| CDI | 1,1′-carbonyldiimidazole |
| COD | 1,5-cyclooctadiene |
| C-Phos or CPhos | 2-dicyclohexylphosphino-2′,6′-bis(N,N- |
| | dimethylamino)biphenyl |
| CuTc | copper(I) thiophene-2-carboxylate |
| DAST | (diethylamino)sulfur trifluoride |
| dba | dibenzylidineacetone |
| DCE | 1,2-dichloroethane |
| DCM | dichloromethane |
| dF(CF₃)ppy | 3,5-difluoro-2-[5-(trifluoromethyl)-2-pyridinyl-N]phenyl-C |
| DIBAL-H | diisobutylaluminum hydride |
| DMAP | 4-dimethylaminopyridine |
| DMEDA | N,N′-dimethylethylenediamine |
| DMF | N,N-dimethylformamide |
| DMSO | dimethyl sulfoxide |
| dppb | 1,4-bis(diphenylphosphino)butane |
| Dppf, DPPF or dppf | 1,l′-bis(diphenylphosphino)ferrocene |
| dtbpf | 1,1′-bis(di-tert-butylphosphino)ferrocene |
| dtbbpy | 4,4′-bis(tert-butyl)-2,2′-bipyridine |
| eq or eq. or equiv. | equivalent |
| ESI or ES | electrospray ionization |
| Et | ethyl |
| EtOAc or EA | ethyl acetate |
| g | gram(s) |
| Grubbs II | (1,3-bis(2,4,6-trimethylphenyl)-2- |
| | imidazolidinylidene)dichloro(phenylmethylene)(tricyclohexyl |
| | phosphine)ruthenium |
| h or hr | hour(s) |
| HATU | hexafluorophosphate azabenzotriazole tetramethyl uronium |
| HFIP | 1,1,1,3,3,3-hexafluoropropan-2-ol |
| HMDS | hexamethyldisilazide |
| Hoveyda-Grubbs II | (1,3-bis-(2,4,6-trimethylphenyl)-2- |
| | imidazolidinylidene)dichloro(o- |
| | isopropoxyphenylmethylene)ruthenium |
| HPLC | high pressure liquid chromatography |
| iPr | isopropyl |
| iPr₂NEt, DIEA, or DIPEA | N-ethyl diisopropylamine (Hunig′s base) |
| LC MS, LCMS, LC-MS or | liquid chromatography mass spectroscopy |
| LC/MS | |
| m/z | mass divided by charge |
| m-CPBA | 3-chloroperbenzoic acid |
| Me | methyl |
| MeCN, ACN, or CH₃CN | acetonitrile |
| MeOH | methanol |
| mg | milligrams |
| min | minutes |
| mL | milliliters |
| MS | mass spectrometry |
| MsOH | methanesulfonic acid |
| MTBE | methyl tert-butyl ether |
| n-BuLi | n-butyllithium |
| NMP | N-methylpyrrolidone |
| NMR | nuclear magnetic resonance |
| Ns | (4-nitrophenyl)sulfonyl |
| OTf or TfO | triflate |
| PE | petroleum ether |
| Ph | phenyl |
| phen | 1,10-phenanthroline |
| Piv or PivO | pivaloyl or pivalate |
| PyBroP | bromotripyrrolidinophosphonium hexafluorophosphate |
| RT or rt or r.t. | room temperature |
| RuPhos Pd G3 | (2-dicyclohexylphosphino-2′,6′-diisopropoxy-1,1′- |
| | biphenyl)[2-(2′-amino-1,1′-biphenyl)]palladium(II) |
| | methanesulfonate |
| sat. | saturated |
| SFC | supercritical fluid chromatography |
| tBu or ^{t}Bu | tert-butyl |
| TEA or Et₃N | triethylamine |
| TES | N-[tris(hydroxymethyl)methyl]-2-aminoethanesulfonic acid |
| TFA | trifluoroacetic acid |
| TFAA | trifluoroacetic anhydride |
| THF | tetrahydrofuran |
| TMP | 2,2,6,6-tetramethylpiperidide |
| TMS | trimethylsilyl |
| TsO or TsOH | tosylate or tosic acid |
| Xantphos Pd G3 | [(4,5-bis(diphenylphosphino)-9,9-dimethylxanthene)-2-(2′- |
| | amino-1,1′-biphenyl)]palladium(II) methanesulfonate |
| XPhos | 2-dicyclohexylphosphino-2′,4′,6′-triisopropylbiphenyl |
| PE | petroleum ether |

### General Analytical and Purification Methods

Provided in this section are descriptions of the general analytical and purification methods used to prepare the specific compounds provided herein.

### Chromatography:

Unless otherwise indicated, crude product-containing residues were purified by passing the crude material or concentrate through either a Biotage brand silica gel column pre-packed with flash silica (SiO2) or reverse phase flash silica (C₁₈) and eluting the product off the column with a solvent gradient as indicated. For example, a description of silica gel (0-40% EtOAc/hexane) means the product was obtained by elution from the column packed with silica using a solvent gradient of 0% to 40% EtOAc in hexanes.

### Preparative HPLC or Reverse Phase Flash Chromatography Purification:

Where so indicated, the compounds described herein were purified via reverse phase HPLC using Waters Fractionlynx semi-preparative HPLC-MS system utilizing one of the following two HPLC columns: (a) Phenominex Gemini column (5 micron, C18, 150×30 mm) or (b) Waters X-select CSH column (5 micron, C18, 100×30 mm).

A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

prep-HPLC or reverse phase flash purification was performed using one of the following conditions:
Condition 1: Column: Phenomenex luna C18 250×50 mm×10 μm; Mobile Phase A: [H₂O] (conditions: [water (0.225% NH₃·H₂O (FA))]); Mobile Phase B: acetonitrile (ACN); Gradient a: 65% B to 90% B;
Condition 2: Column: Phenomenex luna C18 250×50 mm×10 μm; Mobile Phase A: [H₂O] (conditions: [water (0.225% FA)]); Mobile Phase B: ACN; Gradient a: 65% B to 90% B;
Condition 3: Column: Phenomenex luna C18 150×25 mm×10 μm; Mobile Phase A: [H₂O] (conditions: [water (0.225% FA]); Mobile Phase B: ACN; Gradient a: 55% B to 85% B; Gradient b: 48% B to 78% B; Gradient c: 50% to 80%;
Condition 4: Column: YMC-Actus Triart C18 150×30 mm×7 μm; Mobile Phase A: [H₂O] (conditions: [water (0.05% FA)]); Mobile Phase B: ACN;
Condition 5: Column: Welch Xtimate C18 150×25 mm×5 μm; Mobile Phase A: [H₂O] (conditions: [water (0.225% FA)]); Mobile Phase B: ACN; Gradient a: 30% B to 60% B in 40 min; Gradient b: 55% B to 74% B in 40 min; Gradient c: 50% B to 80% B in 40 min; Gradient d: 35% B to 65% B in 40 min; Gradient e: 40% B to 70% B in 35 min; Gradient f: 35% B to 75% B in 40 min; Gradient g: 45% B to 75% B in 12 min, Mobile Phase A: [H₂O (0.1% FA)];
Condition 6: Column: DAICEL CHIRALCEL OJ (250 mm×50 mm, 10 μm); Mobile Phase A: 0.1% NH₃·H₂O in ethanol (EtOH); Flow Rate (150 mL/min); Gradient a: 0% B in 8.5 min;
Condition 7: Column: Phenomenex luna C18 150×25 mm×10 μm; Mobile Phase A: [H₂O] (conditions: [water (0.1% FA)]); Mobile Phase B: ACN; Flow Rate: 25 mL/min; Gradient a: 38% B to 68% B in 10 min; Gradient b: 35% B to 65% B in 10 min; Gradient c: 48% B to 78% B in 7 min; Gradient d: 50% B to 80% B in 7 min;
Condition 8: Column: Phenomenex luna C18 150×40 mm×15 μm; Mobile Phase A: [H₂O] (conditions: [water (0.1% FA)]); Mobile Phase B: ACN; Flow Rate: 60 mL/min; Gradient a: 25% B to 55% B;
Condition 9: Column: 12 g C18 column; Mobile Phase A: [H₂O] (conditions: [water (0.1% FA)]); Mobile Phase B: ACN; Gradient a: 10% B to 90% B; Gradient b: 30% B to 95% B; Gradient c: 15% B to 100% B; Gradient d: 30% B to 80% B;
Condition 10: Column: Gemini 5 μm NX—C18 110 Å, 100×30 mm column; Mobile Phase A: [H₂O] (conditions: [water (10 mM NH₄HCO₂)]); Mobile Phase B: ACN; Gradient a: 40% B to 100% B; Gradient b: 60% B to 100% B; Gradient c: 35% B to 50% B; Gradient d: 45% B to 65% B; Gradient e: 30% B to 100% B; Gradient f: 55% B to 100% B;
Condition 11: Column: 12 g C18 column; Mobile Phase A: [H₂O] (conditions: [water (10 mM ammonium bicarbonate)]); Mobile Phase B: ACN; Gradient a: 15% B to 80% B;
Condition 12: Column: 6 g Biotage Reverse Phase; Mobile Phase A: [H₂O] (conditions: [water (0.1% FA)]); Mobile Phase B: ACN; Gradient a: 5% B to 95% B;
Condition 13: Column: 18 g Biotage Reverse Phase; Mobile Phase A: [H₂O] (conditions: [water (0.1% FA)]); Mobile Phase B: ACN; Gradient a: 15% B to 85% B;
Condition 14: Column: Gemini 5 μm NX—C18 110 Å, 100×30 mm column; Mobile Phase A: [H₂O] (conditions: [water (0.1% FA)]); Mobile Phase B: MeOH; Gradient a: 25% B to 45% B; Gradient b: 60% B to 80% B; Gradient c: 50% B to 70% B;
Condition 15: Column: Gemini 5 μm NX—C18 110 Å, 100×30 mm column; Mobile Phase A: [H₂O] (conditions: [water (10 mM ammonium bicarbonate)]); Mobile Phase B: MeOH; Gradient a: 45% B to 55% B;
Condition 16: Column: Gemini 5 μm NX—C18 110 Å, 100×30 mm column; Mobile Phase A: [H₂O] (conditions: [water (10 mM NH₄HCO2)]); Mobile Phase B: MeOH; Gradient a: 50% B to 70% B; Gradient b: 45% B to 100% B; Gradient c: 55% B to 75% B;
Condition 17: Column: Gemini 5 μm NX—C18 110 Å, 100×30 mm column; Mobile Phase A: [H₂O] (conditions: [water (0.1% FA)]); Mobile Phase B: MeCN; Gradient a: 10% B to 95% B;
Condition 18: Column: Boston Green ODS, 150×30 mm×5 μm; Mobile Phase A: [H₂O] (conditions: [water (0.225% FA)]); Mobile Phase B: MeCN; Gradient a: 45% B to 75% B; Gradient b: 38% B to 68% B. Gradient c: 48% B to 78% B;
Condition 19: Column: Phenomenex luna C18 250×70 mm×10 μm; Mobile Phase A: [H₂O] (conditions: [water (0.225% FA)]); Mobile Phase B: ACN; Gradient a: 30% B to 60% B;

### Preparative Normal Phase Liquid Chromatography (Prep-NPLC):

Condition 1: Column: Welch Ultimate XB—NH₂ 250×50×10 μm, Hexane-EtOH;

### Analytical HPLC Method:

Where so indicated, the compounds described herein were analyzed using an Aglilent 1100 series instrument with DAD detector.

### Flash Chromatography Method:

Where so indicated, flash chromatography was performed on Teledyne Isco instruments using pre-packaged disposable SiO₂ stationary phase columns with eluent flow rate range of 15 to 200 mL/min, UV detection (254 and 220 nm).

### Preparative Chiral Supercritical Fluid Chromatography (SFC) Method:

Where so indicated, the compounds described herein were purified via chiral SFC using one of the two following chiral SFC columns: (a) Chiralpak IG 2×25 cm, 5 μm or (b) Chiralpak AD-H 2×15 cm, 5 μm.

Some CP Analytical-SFC experiments were run on SFC Method Station (Thar, Waters) with the following conditions: Column temperature: 40° C., Mobile phase: CO₂/Methanol (0.2% Methanol Ammonia)=Flow: 4.0 mL/min, Back Pressure: 120 Bar, Detection wavelength: 214 nm.

Some CP Analytical-SFC experiments were run on SFC-80 (Thar, Waters) with the following conditions: Column temperature: 35° C., Mobile phase (example): CO₂/Methanol (0.2% Methanol Ammonia)=Flow rate: 80 g/min, Back pressure: 100 bar, Detection wavelength: 214 nm.

Preparative CP Method: Acidic reversed phase MPLC: Instrument type: Reveleris™ prep MPLC; Column: Phenomenex LUNA C18 (3)(150×25 mm, 10μ); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.
Condition 1: Column: Chiralcel OJ-3, 50×4.6 mm, 3 μm; Mobile Phase A: CO₂; Mobile Phase B: methanol (MeOH)(0.05% diethanolamine (DEA)); Flow Rate: 3 mL/min; Column Temp: 35° C.; Back Pressure: 100 Bar; Gradient a: 5% B to 40% B; Gradient a-1: 5% B to 40% B, Mobile Phase B: EtOH (0.05% DEA); Gradient a-2: 5% B to 40% B, Mobile Phase B: isopropyl alcohol (iPOH)(0.05% DEA);
Condition 2: Column: Chiralpak AD-3, 50×4.6 mm, 3 μm; Mobile Phase A: CO₂; Mobile Phase B: MeOH (0.05% DEA); Flow Rate: 3 mL/min; Column Temp: 35° C.; Back Pressure: 100 Bar; Gradient a: 40% B;
Condition 3: Column: DAICEL Chiralpak IG, 250 mm×50 mm, 10 μm; Mobile Phase A: CO₂; Mobile Phase B: EtOH/ACN; Flow Rate: 100 mL/min; Column Temp: 35° C.; Back Pressure: 100 Bar; Gradient a: 75% B in 8.1 min; Gradient b: 75% B in 7.5 min;
Condition 4: Column: Chiralpak AD-3, 50×4.6 mm, 3 μm; Mobile Phase A: CO₂; Mobile Phase B: EtOH (0.05% DEA); Flow Rate: 3 mL/min; Column Temp: 35° C.; Back Pressure: 100 Bar; Gradient a: 5% B to 40% B; b: 40% B;
Condition 5: Column: Chiralpak IG-3, 50×4.6 mm, 3 μm; Mobile Phase A: CO₂; Mobile Phase B: EtOH (0.05% DEA); Flow Rate: 3 mL/min; Column Temp: 35° C.; Back Pressure: 100 Bar; Gradient a: 40% B; Gradient a-1: 40% B, Mobile Phase B: MeOH (0.05% DEA); Gradient a-2: 40% B, Mobile Phase B: iPOH+ACN (0.05% DEA); Gradient b: 60% B, Mobile Phase B: iPOH+ACN (0.05% DEA); Gradient c: 50% B;
Condition 6: Column: DAICEL Chiralpak AD, 250 mm×30 mm, 10 μm; Mobile Phase A: CO₂; Mobile Phase B: EtOH (0.1% NH₃·H₂O); Flow Rate: 150 mL/min; Column Temp: 35° C.; Back Pressure: 100 Bar; Gradient a: 23% B in 5.3 min; Gradient b: 25% B in 2.5 min; Gradient c: 3.9 min; Gradient d: 5.0 min; Gradient e: 34% B in 6.5 min; Gradient f: 4.4 min, Mobile Phase B: ACN/(+) i-PrOH (0.1% NH₃·H₂O), Flow Rate: 120 mL/min;
Condition 7: Column: DAICEL Chiralpak AD, 250 mm×50 mm, 10 μm; Mobile Phase A: CO₂; Mobile Phase B: i-PrOH (0.1% NH₃·H₂O); Flow Rate: 150 mL/min; Column Temp: 35° C.; Back Pressure: 100 Bar; Gradient a: 35% B in 5 min;
Condition 8: Column: DAICEL Chiralpak IH, 250 mm×50 mm, 10 μm; Mobile Phase A: CO₂; Mobile Phase B: EtOH (0.1% NH₃·H₂O); Flow Rate: 150 mL/min; Column Temp: 35° C.; Back Pressure: 100 Bar; Gradient a: 27% B in 5.5 min;
Condition 9: Column: DAICEL Chiralpak OD-3, 50 mm×4.6 mm, 3 μm; Mobile Phase A: CO₂; Mobile Phase B: MeOH (0.5% DEA); Flow Rate: 3 mL/min; Column Temp: 35° C.; Back Pressure: 100 Bar; Gradient a: 5% B to 40% B;
Condition 10: Column: DAICEL Chiralpak AD (250 mm×30 mm, 10 μm); Mobile Phase A: MeOH (0.1% NH₃·H₂O); Flow Rate (100 mL/min); Gradient a: 0% B in 8.2 min;
Condition 11: Column: Chiralpak IG-3, 250 mm×30 mm, 10 μm; Mobile Phase A: CO₂; Mobile Phase B: MeOH (0.1% NH₃·H₂O); Flow Rate: 150 mL/min; Column Temp: 35° C.; Back Pressure: 100 Bar; Gradient a: 35% B; Gradient b: 56% B, Mobile Phase B: EtOH; Gradient c: 40% B, Flow Rate: 120 mL/min; Gradient d: 40% B, Mobile Phase B: MeOH; Gradient e: 35% B, Mobile Phase B: iPrOH (0.1% NH₃. H₂O)
Condition 12: Column: Chiralcel OJ, 250 mm×30 mm, 10 μm; Mobile Phase A: CO₂; Mobile Phase B: MeOH (0.1% NH₃. H₂O); Flow Rate: 120 mL/min; Column Temp: 35° C.; Back Pressure: 100 Bar; Gradient a: 30% B;
Condition 13: Column: Chiralpak AD-3, 50×4.6 mm, 3 μm; Mobile Phase A: CO₂; Mobile Phase B: IPA+ACN (0.05% DEA); Flow Rate: 3 mL/min; Column Temp: 35° C.; Back Pressure: 100 Bar; Gradient a: 40% B;
Condition 14: Column: DAICEL Chiralpak IE-3, 50 mm×4.6 mm, 3 μm; Mobile Phase A: CO₂; Mobile Phase B: IPA+ACN (0.05% DEA); Flow Rate: 3 mL/min; Column Temp: 35° C.; Back Pressure: 100 Bar; Gradient a: 40% B;
Condition 15: Column: DAICEL CHIRALPAK IF, 50 mm×4.6 mm, 3 μm; Mobile Phase A: CO₂; Mobile Phase B: EtOH (0.05% DEA); Flow Rate: 3 mL/min; Column Temp: 35° C.; Back Pressure: 100 Bar; Gradient a: 40% B;
Condition 16: Column: DAICEL Chiralpak IG-3, 50 mm×4.6 mm, 3 μm; Mobile Phase A: CO₂; Mobile Phase B: IPA+ACN (0.05% DEA); Flow Rate: 3 mL/min; Column Temp: 35° C.; Back Pressure: 100 Bar; Gradient a: 60% B;
Condition 17: Column: Chiralcel OD-3, 50 mm×4.6 mm, 3 μm; Mobile Phase A: CO₂; Mobile Phase B: IPA (0.05% DEA); Flow Rate: 3 mL/min; Column Temp: 35° C.; Back Pressure: 100 Bar; Gradient a: 5% B to 40% B;
Condition 18: Column: DAICEL Chiralpak IG-3, 50 mm×4.6 mm, 3 μm; Mobile Phase A: CO₂; Mobile Phase B: IPA (0.05% DEA); Flow Rate: 3 mL/min; Column Temp: 35° C.; Back Pressure: 100 Bar; Gradient a: 60% B;
Condition 19: Column: Chiralpak AD-3, 50×4.6 mm, 3 μm; Mobile Phase A: CO₂; Mobile Phase B: IPA (0.05% DEA); Flow Rate: 3 mL/min; Column Temp: 35° C.; Back Pressure: 100 Bar; Gradient a: 5% B to 40% B;
Condition 20: Column: DAICEL Chiralcel OD, 250×30 mm, 10 μm; Mobile Phase A: CO₂; Mobile Phase B: EtOH (0.1% NH₃H₂O); Flow Rate: 150 mL/min; Column Temp: 35° C.; Back Pressure: 100 Bar; Gradient a: 30% B;
Condition 21: Column: Phenomenex-Cellulose-2, 250×30 mm, 10 μm; Mobile Phase A: CO₂; Mobile Phase B: EtOH (0.1% NH₃H₂O); Flow Rate: 150 mL/min; Column Temp: 35° C.; Back Pressure: 100 Bar; Gradient a: 45% B;

### Proton NMR Spectra:

Unless otherwise indicated, all ¹H NMR spectra were collected on a Bruker NMR Instrument at 300, 400 or 500 Mhz or a Varian NMR Instrument at 400 Mhz. Where so characterized, all observed protons are reported as parts-per-million (ppm) downfield from tetramethylsilane (TMS) using the internal solvent peak as reference. All NMR were collected at about 25° C.

### Mass Spectra (MS)

Unless otherwise indicated, all mass spectral data for starting materials, intermediates and/or exemplary compounds are reported as mass/charge (m/z), having an [M+H]⁺ molecular ion. The molecular ion reported was obtained by electrospray detection method (commonly referred to as an ESI MS) utilizing a Waters Acquity UPLC/MS system or a Gemini-NX UPLC/MS system. Compounds having an isotopic atom, such as bromine and the like, are generally reported according to the detected isotopic pattern, as appreciated by those skilled in the art.

### Compound Names

The compounds disclosed and described herein have been named using the IUPAC naming function of ChemDraw Professional 17.0.

### SPECIFIC EXAMPLES

Provided in this section are the procedures to synthesize specific examples of the compounds provided herein. All starting materials are either commercially available from Sigma-Aldrich Inc., unless otherwise noted, or known in the art and may be synthesized by employing known procedures using ordinary skill.

### Example A1: Synthesis of Intermediates

### Method Int 1. Intermediate Int-A1 5-acetyl-1-methyl-pyridin-2-one

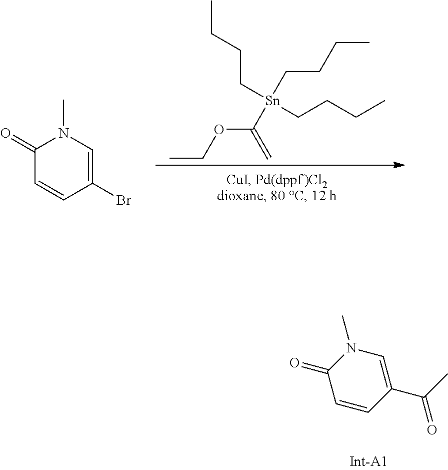

To a solution of 5-bromo-1-methyl-pyridin-2-one (1 equiv., 6.9 g, 36.7 mmol) in 1,4-dioxane (130 mL) was added Pd(dppf)Cl₂ (0.1 equiv., 2.7 g, 3.67 mmol), CuI (0.2 equiv., 1.4 g, 7.34 mmol) and tributyl(1-ethoxyvinyl) stannane (1.1 equiv., 14 mL, 40.4 mmol), and the reaction mixture was allowed to stir at 80° C. for 12 hours under nitrogen. LCMS showed 32% of desired mass. Aqueous (aq.) saturated KF (20 mL) was added and the mixture was stirred at 25° C. for 1 hour. The resulting mixture was extracted with ethyl acetate (EtOAc)(3×100 mL), and the combined organic layers were washed with aq saturated NaCl (50 mL), dried over anhydrous Na₂SO₄, filtered, and concentrated under reduced pressure to give a crude residue. The residue was purified by prep-HPLC (Condition 1, Gradient a) and lyophilized to afford 5-acetyl-1-methyl-pyridin-2-one (Int-A1, 3 g, 18.9 mmol, 51% yield) as a solid. LCMS: [M+H]⁺=152.0; purity=95% (220 nm); Ret. time=0.179 min.

### Method Int 2. Intermediate Int-A2:5-(2-bromoacetyl)-1-methyl-pyridin-2-one

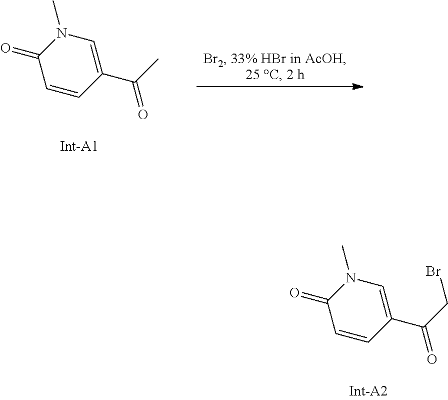

To a solution of 5-acetyl-1-methyl-pyridin-2-one (Int-A1, 1 equiv., 700 mg, 4.6 mmol) in 33% HBr in acetic acid (AcOH)(29 equiv., 10.9 g, 136 mmol), was added Br₂ (1 equiv., 0.24 mL, 4.6 mmol)/33% HBr in AcOH (7.4 equiv., 2.7 g, 34 mmol) dropwise at 0° C. slowly, then stirred at 25° C. for 2 hours. LCMS showed 51% of desired product. The reaction mixture was extracted with EtOAc (3×100 mL). The combined organic layers were dried over Na₂SO₄, filtered, and concentrated under reduced pressure to give a crude residue which was purified by silica gel column chromatography (100% EtOAc) (TLC, 100% EtOAc, R_{f}=0.50) to afford 5-(2-bromoacetyl)-1-methyl-pyridin-2-one (Int-A2, 1.7 g, 6.9 mmol, 99% yield) as a solid. ¹H NMR (400 MHz, CDCl₃) δ:8.25 (d, J=2.6 Hz, 1H), 7.87 (dd, J=2.6, 9.6 Hz, 1H), 6.59 (d, J=9.6 Hz, 1H), 4.20 (s, 2H), 3.65 (s, 3H).

### Method Int 3. Intermediate Int-A3: N-[(2R)-2-hydroxypropyl]-N-[2-(1-methyl-6-oxo-3-pyridyl)-2-oxo-ethyl]-4-nitro-benzenesulfonamide

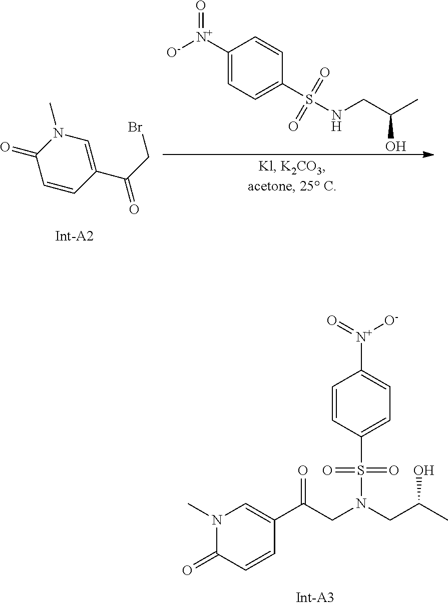 [SMILES: Cn1cc(C(=O)CBr)ccc1=O ; C[C@@H](O)CNS(=O)(=O)c1ccc([N+](=O)[O-])cc1 ; C[C@@H](O)CN(CC(=O)c1ccc(=O)n(C)c1)S(=O)(=O)c1ccc([N+](=O)[O-])cc1]

To a solution of 5-(2-bromoacetyl)-1-methyl-pyridin-2-one (Int-A2, 1 equiv., 700 mg, 3 mmol) and N-[(2R)-2-hydroxypropyl]-4-nitro-benzenesulfonamide (1 equiv., 792 mg, 3 mmol) in Acetone (15 mL) was added K₂CO₃ (3 equiv., 1262 mg, 9 mmol) and KI (1.1 equiv., 556 mg, 3.3 mmol), and the reaction was stirred at 25° C. for 12 hours. LCMS showed 73% desired product. The reaction mixture was filtered through a pad of Celite. The filter cake was washed with EtOAc (3×5 mL) and then extracted with EtOAc (3×50 mL). The combined organic layers were dried over Na₂SO₄, filtered, and concentrated under reduced pressure to give a crude residue. The crude residue was purified by silica gel column chromatography (10% dichloromethane (DCM) in MeOH) to afford N-[(2R)-2-hydroxypropyl]-N-[2-(1-methyl-6-oxo-3-pyridyl)-2-oxo-ethyl]-4-nitro-benzenesulfonamide (Int-A3, 400 mg, 0.87 mmol, 29% yield) as a solid. LCMS: [M+H]⁺=410.0; purity=89% (220 nm); Ret. time=0.485 min.

### Method Int 4. Intermediate Int-A4:1-methyl-5-[(2S,6R)-6-methyl-4-(4-nitrophenyl) sulfonyl-morpholin-2-yl]pyridin-2-one

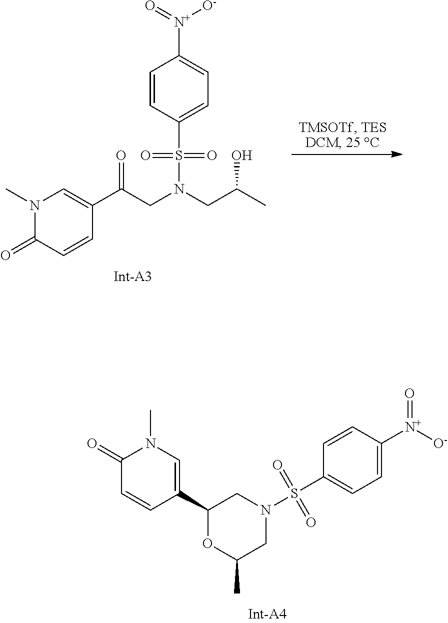 [SMILES: C[C@@H](O)CN(CC(=O)c1ccc(=O)n(C)c1)S(=O)(=O)c1ccc([N+](=O)[O-])cc1 ; "TMSOTf, TESDCM, 25 °C" ; C[C@@H]1CN(S(=O)(=O)c2ccc([N+](=O)[O-])cc2)C[C@H](c2ccc(=O)n(C)c2)O1]

To a solution of N-[(2R)-2-hydroxypropyl]-N-[2-(1-methyl-6-oxo-3-pyridyl)-2-oxo-ethyl]-4-nitro-benzenesulfonamide (Int-A3, 1 equiv., 400 mg, 0.98 mmol) in DCM (4 mL) was added TES (10 equiv., 1.4 mL, 9.8 mmol), then TMSOTf (10 equiv., 1.8 mL, 9.8 mmol) was added slowly at 0° C., and the reaction was stirred at 25° C. for 12 hours. LCMS showed 61% desired product. The reaction mixture was poured into saturated 50% NaOH aqueous solution (10 mL) until pH=14, then stirred at 60° C. for 2 hours. The mixture was dissolved totally, extracted with DCM (3×20 mL), and the combined organic layers were dried over Na₂SO₄, filtered, and concentrated under reduced pressure to give 1-methyl-5-[(2S,6R)-6-methyl-4-(4-nitrophenyl) sulfonyl-morpholin-2-yl]pyridin-2-one (Int-A4, 400 mg, 0.81 mmol, 83% yield) as a solid. LCMS: [M+H]⁺=393.9; purity=82% (220 nm); Ret. time=0.847 min.

### Method Int 5. Intermediate Int-A5:1-methyl-5-[(2S,6R)-6-methylmorpholin-2-yl]pyridin-2-one

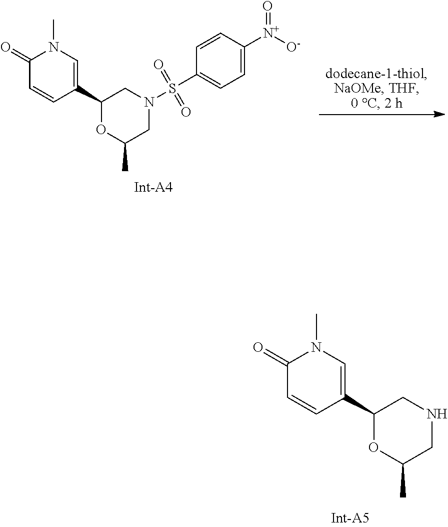 [SMILES: C[C@@H]1CN(S(=O)(=O)c2ccc([N+](=O)[O-])cc2)C[C@H](c2ccc(=O)n(C)c2)O1 ; "dodecane-1-thiol,NaOMe, THF,0 °C, 2 h" ; C[C@@H]1CNC[C@H](c2ccc(=O)n(C)c2)O1]

Under N₂ atmosphere, to a solution of 1-methyl-5-[(2S,6R)-6-methyl-4-(4-nitrophenyl) sulfonyl-morpholin-2-yl]pyridin-2-one (Int-A4, 1 equiv., 350 mg, 0.71 mmol) in THF (15 mL) cooled to 0~5° C., was charged dodecane-1-thiol (2.5 equiv., 13 mg, 0.063 mmol) dropwise at 0~5° C. The reaction mixture was agitated at 0~5° C. for 0.5 h under N₂ atmosphere, then sodium methanolate (3 equiv., 115 mg, 2.14 mmol) was added into the solution at 0~5° C. The resulting mixture was agitated for 1 hour at 0~5° C. under N₂ protection. LCMS showed 21% desired product. The reaction mixture was poured into saturated 50% NaOH aqueous solution (10 mL) until pH=14, then stirred at 25° C. for 1 hour. The mixture was dissolved totally, extracted with DCM (3×20 mL), then the combined organic layers were dried over Na₂SO₄, filtered, and concentrated under reduced pressure to give 1-methyl-5-[(2S,6R)-6-methylmorpholin-2-yl]pyridin-2-one (Int-A5, 70 mg, 0.27 mmol, 38% yield) as a solid. LCMS: [M+H]⁺=209.5; purity=100% (220 nm); Ret. time=0.123 min.

### Method Int 6. Intermediate Int-A6:2-chloro-6,7-dimethyl-4-methylsulfanyl-pteridine

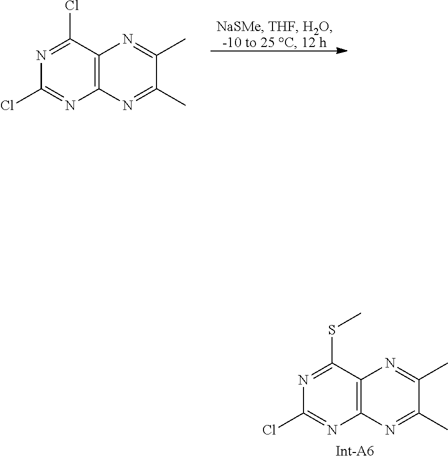

To a solution of 2,4-dichloro-6,7-dimethyl-pteridine (1 equiv., 1.5 g, 6.6 mmol) in THF (15 mL), was added NaSMe (1.2 equiv., 551 mg, 7.9 mmol)(dissolved in water (5 mL)) dropwise at −10° C., and the reaction mixture was stirred at 25° C. for 14 hours. LCMS showed 76% of desired product. The reaction mixture was poured into H₂O (50 mL) and then extracted with EtOAc (3×80 mL). The combined organic layers were dried over Na₂SO₄, filtered, and concentrated under reduced pressure to give a residue. The residue was purified by silica gel column chromatography (25% EtOAc in petroleum ether (PE), R_{f}=0.3) to afford 2-chloro-6,7-dimethyl-4-methylsulfanyl-pteridine (Int-A6, 800 mg, 3.3 mmol, 51% yield) as a solid. LCMS: Ret. Time=0.504 min, [M+H]⁺=240.8 ESI+.

### Method Int 7. Intermediate Int-A7:5-[(2S,6R)-4-(6,7-dimethyl-4-methylsulfanyl-pteridin-2-yl)-6-methyl-morpholin-2-yl]-1-methyl-pyridin-2-one

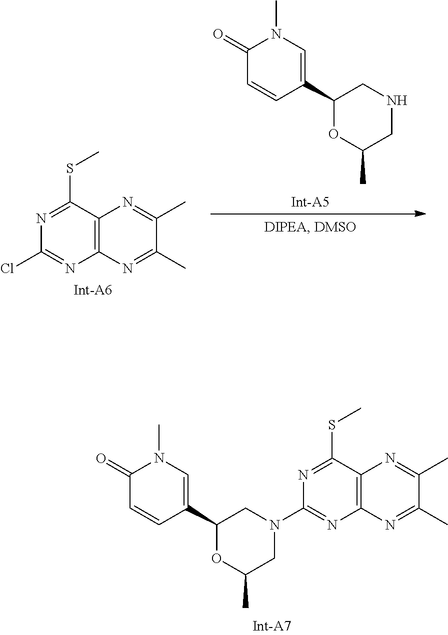

To a solution of 2-chloro-6,7-dimethyl-4-methylsulfanyl-pteridine (Int-A6, 1 equiv., 35 mg, 0.15 mmol) and 1-methyl-5-[(2S, 6R)-6-methylmorpholin-2-yl]pyridin-2-one (1.2 equiv., 36 mg, 0.17 mmol) in DMSO (1 mL), was added DIPEA (5 equiv., 94 mg, 0.73 mmol) at 100° C. for 2 h. LCMS showed 88% of desired product. The reaction mixture was combined with another batch for further purification. The mixture was diluted with water (50 mL) and extracted with EtOAc (3×50 mL). The combined organic layers were washed with brine, dried over Na₂SO₄, filtered, and concentrated under reduced pressure to give a residue. The residue was purified by reversed-phase HPLC (50% [water (0.1% FA)-ACN], in 5 min) to afford 5-[(2S,6R)-4-(6,7-dimethyl-4-methylsulfanyl-pteridin-2-yl)-6-methyl-morpholin-2-yl]-1-methyl-pyridin-2-one (Int-A7, 86 mg, 0.21 mmol, 141% yield) as an oil. LCMS: Ret. Time=0.487 min, [M+H]⁺=413.0, ESI+, 98% purity (220 nm).

### Method Int 8. Intermediate Int-A8: bromo-[2-(1-methyl-6-oxo-3-pyridyl)tetrahydropyran-4-yl]zinc

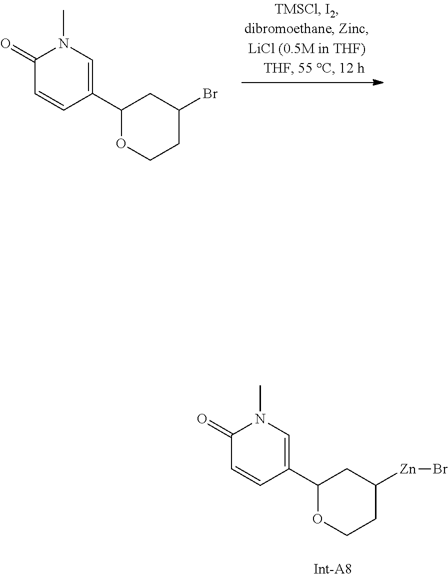

The synthesis was performed at a 2.4 g scale but 2 batches of 1.2 g was performed in parallel. The 1.2 g procedure was shown as below:

To a solution of zinc (3 equiv., 865 mg, 13 mmol) suspended in LiCl (0.5 M in THF)(1 equiv., 9.0 mL, 4.4 mmol), was added 1,2-dibromoethane (0.05 equiv., 0.019 mL, 0.22 mmol) and the suspension was stirred at 55° C. for 20 min. The resulting mixture was then cooled, TMSCl (0.05 equiv., 0.028 mL, 0.22 mmol) was introduced and the mixture was stirred for another 20 mins. The resulting mixture was cooled, then iodine (0.02 equiv., 22 mg, 0.09 mmol) in THF (1 mL) was introduced and the reaction was stirred at 55° C. for another 20 mins. 5-(4-bromotetrahydropyran-2-yl)-1-methyl-pyridin-2-one (1 equiv., 1.2 g, 4.4 mmol) in THF (9 mL) was then added to the warm suspension of activated zinc, and the reaction solution was stirred at 55° C. for 12 hours. The reaction mixture was used directly for the next step via syringe transfer.

### Method Int 9. Intermediate Int-A10: chloro-[2,3-difluoro-4-(trifluoromethyl)phenyl]zinc

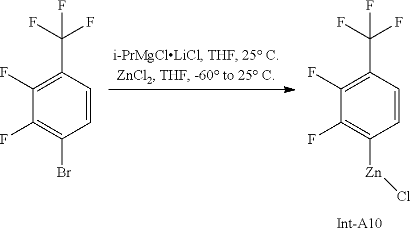

A solution of 1-bromo-2,3-difluoro-4-(trifluoromethyl)benzene (1 equiv., 350 mg, 1.34 mmol) in THF (4 mL) was added i-PrMgCl·LiCl (1.1 equiv., 1.1 mL, 1.48 mmol) slowly at 25° C. under N₂ and stirred for 1 h at 25° C. The solution changed color. The mixture was cooled to-60° C. and then ZnCl₂ (1.1 equiv., 3.0 mL, 1.48 mmol) was added to the mixture and stirred for 1 h at 25° C. The solution changed color, and the mixture (Int-A10) was used directly for next step.

### Method Int 10. Intermediate Int-A11:2-chloro-4-[2,3-difluoro-4-(trifluoromethyl)phenyl]-6,7-dimethyl-pteridine

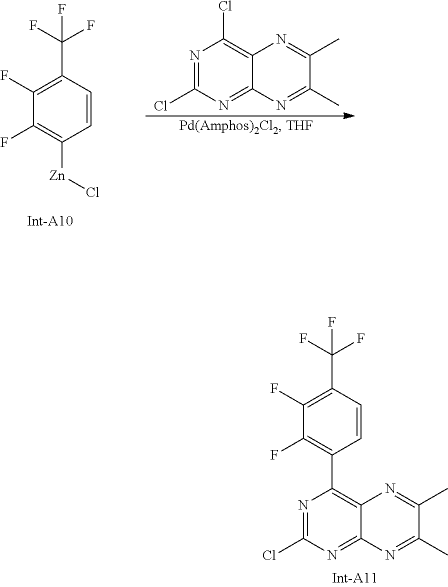

To a solution of 2,4-dichloro-6,7-dimethyl-pteridine (0.814 equiv., 250 mg, 1.09 mmol) and Pd(Amphos)₂Cl₂ (0.0326 equiv., 31 mg, 0.0437 mmol) in THF (3 mL) was added chloro-[2,3-difluoro-4-(trifluoromethyl)phenyl]zinc (Int-A10, 1 equiv., 378 mg, 1.34 mmol), and the reaction mixture was stirred for 2 h at 25° C. LCMS showed the raw materials were consumed and the major peak showed the desired mass. The reaction mixture was added water (10 mL) and extracted with EtOAc (3×10 mL), the combined organic layer was washed with 10 mL saturated brine solution, separated, dried over Na₂SO₄, filtered, and concentrated under reduced pressure to give a crude which was then purified by prep-HPLC (FA) to give 2-chloro-4-[2,3-difluoro-4-(trifluoromethyl)phenyl]-6,7-dimethyl-pteridine (Int-A11, 180 mg, 0.480 mmol, 36% yield) as a solid. LCMS: [M+H]⁺=375.2, purity=77%.

An analogous method was followed to obtain the following intermediate.

| Structure | Starting Materials | Characterization |
|---|---|---|
| 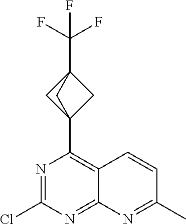 [SMILES: Cc1ccc2c(C34CC(C(F)(F)F)(C3)C4)nc(Cl)nc2n1] Int-B18 | 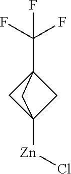 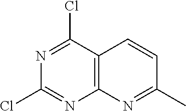 | The crude product was purified by silica gel column chromatography (0-33% EtOAc in PE) to give 2- chloro-7-methyl-4-[3-(trifluoromethyl)-1- bicyclo[1.1.1]pentanyl]pyrido[2,3-d]pyrimidine (Int- B18, 250 mg, 0.797 mmol, 56% yield) as a solid. LCMS: [M + H]⁺ = 314.2; purity = 91% (220 nm); Ret. time = 0.542 min. ¹H NMR (400 MHz, CDCl₃) δ 8.46 (d, J = 8.5 Hz, 1H), 7.47 (d, J = 8.5 Hz, 1H), 2.83 (s, 3H), 2.66 (s, 6H). |

### Method Int 11. Intermediate Int-A12:2-bromo-5-nitro-6-(3-(trifluoromethyl) bicyclo[1.1.1]pentan-1-yl)pyrimidin-4-amine

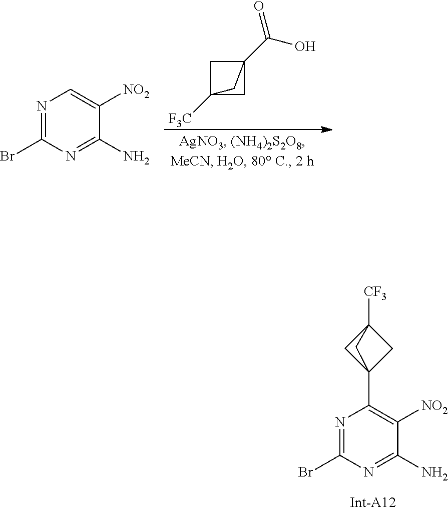 [SMILES: Nc1nc(Br)ncc1[N+](=O)[O-] ; O=C(O)C12CC(C(F)(F)F)(C1)C2 ; Nc1nc(Br)nc(C23CC(C(F)(F)F)(C2)C3)c1[N+](=O)[O-]]

To a solution of 2-bromo-5-nitropyrimidin-4-amine (1 equiv., 500 mg, 2.28 mmol) in MeCN (1 mL) and water (0.5 mL) was added (NH₄)₂S₂O₈ (3 equiv., 1.56 g, 6.85 mmol), AgNO₃ (1.3 equiv., 501 mg, 2.97 mmol) and 3-(trifluoromethyl) bicyclo[1.1.1]pentane-1-carboxylic acid (1.2 equiv., 493 mg, 2.74 mmol), and the reaction mixture was stirred at 80° C. for 2 h. LCMS showed the starting materials were consumed completely, and the desired mass was detected (63%). The mixture was diluted with water (20 mL) and extracted with EtOAc (2×30 mL). The combined organic layers were washed with aqueous brine (3× 40 mL), dried over Na₂SO₄, filtered, and concentrated under reduced pressure to give a residue. The residue was purified by silica gel column chromatography (0% to 25% EtOAc in PE) (TLC, 25% EtOAC in PE, desired product Rr-0.5) to give 2-bromo-5-nitro-6-(3-(trifluoromethyl) bicyclo[1.1.1]pentan-1-yl)pyrimidin-4-amine (Int-A12, 500 mg, 1.42 mmol, 62% yield) as a solid. LCMS: [M+H]⁺=353.0; purity=98% (220 nm); Ret. time=0.586 min. ¹H NMR (400 MHz, CDCl₃) δ 7.01-6.14 (m, 2H), 2.44 (s, 6H).

An analogous procedure was followed to obtain the following intermediates.

| Structure | Starting Material | Characterization |
|---|---|---|
| 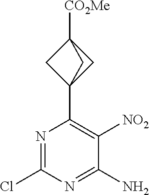 [SMILES: COC(=O)C12CC(c3nc(Cl)nc(N)c3[N+](=O)[O-])(C1)C2] | 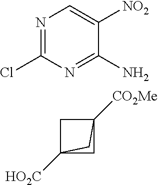 | Modifications: The ammonium persulfate (3.3 equiv.) was added after 5 min of vigorous stirring at 80 °C. The reaction mixture was extracted three times with DCM, and the crude product was purified by normal phase chromatography (0-4% MeOH in DCM) to afford methyl 3-(6-amino-2-chloro-5-nitro-pyrimidin-4- yl)bicyclo[1.1.1]pentane-1-carboxylate (Int-C46, 1.06 g, 3.55 mmol, 41% yield) as a solid. ESI-MS: [M + H]⁺ = 299.1. ¹H NMR (CD₃OD, 400 MHz) δ 3.66 (s, 3H), 2.37 (s, 6H). |
| Int-C46 | | |

### Method Int 12. Intermediate Int-A13:2-bromo-6-(3-(trifluoromethyl) bicyclo[1.1.1]pentan-1-yl)pyrimidine-4,5-diamine

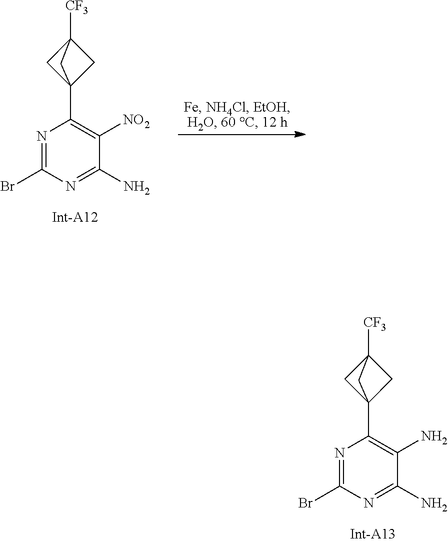 [SMILES: Nc1nc(Br)nc(C23CC(C(F)(F)F)(C2)C3)c1[N+](=O)[O-] ; "Fe, NH4Cl, EtOH,H2O, 60 °C, 12 h" ; Nc1nc(Br)nc(C23CC(C(F)(F)F)(C2)C3)c1N]

To a solution of 2-bromo-5-nitro-6-(3-(trifluoromethyl) bicyclo[1.1.1]pentan-1-yl)pyrimidin-4-amine (Int-A12, 1 equiv., 540 mg, 1.53 mmol) in EtOH (10 mL) and water (2 mL), was added Fe (powder, 5 equiv., 427 mg, 7.65 mmol) and NH₄Cl (6 equiv., 491 mg, 9.18 mmol), and the reaction mixture was stirred at 60° C. for 12 hours. LCMS showed that desired product (85%). The reaction mixture was filtered and concentrated under reduced pressure to give 2-bromo-6-(3-(trifluoromethyl) bicyclo[1.1.1]pentan-1-yl)pyrimidine-4,5-diamine (Int-A13, 495 mg, 1.53 mmol, 100% yield) as a solid. LCMS: [M+H]⁺=325.1; purity=90% (220 nm); Ret. time=0.484 min.

An analogous procedure was followed to obtain the following intermediates.

| Structure | Starting Material | Characterization |
|---|---|---|
| 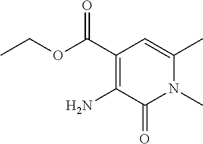 | 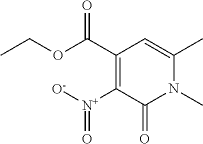 | Modifications: The reaction was stirred at 80 °C for 3 h to obtain ethyl 3-amino-1,6-dimethyl-2-oxo-pyridine-4- carboxylate (Int-D29, 9.00 g, 42.8 mmol, 93% yield) as a solid. LCMS: [M + H]⁺ = 211.1. ¹H NMR (400 MHz, DMSO-d₆) δ 6.66 (br s, 2H), 6.31 (d, J = 0.9 Hz, 1H), 4.25 (q, J = 7.1 Hz, 2H), 3.46 (s, 3H), 2.25 (d, J = 0.7 Hz, 3H), 1.29 (t, J = 7.1 Hz, 3H). |
| Int-D29 | Int-D28 | |

### Method Int 13. Intermediate Int-A14:2-bromo-6,7-dimethyl-4-(3-(trifluoromethyl) bicyclo[1.1.1]pentan-1-yl) pteridine

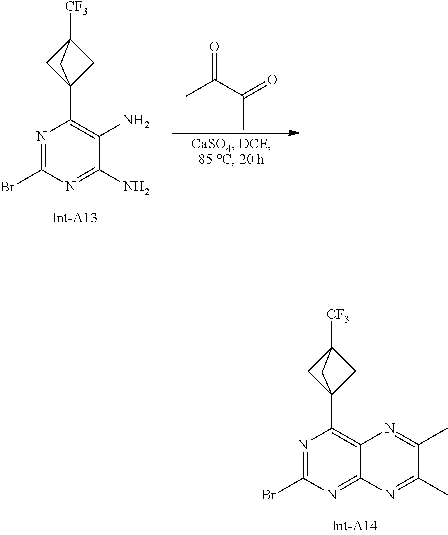

To a mixture of 2-bromo-6-(3-(trifluoromethyl) bicyclo[1.1.1]pentan-1-yl)pyrimidine-4,5-diamine (Int-A13, 1 equiv., 495 mg, 1.53 mmol) suspended in DCE (10 mL), was added CaSO₄ (5.2 equiv., 1085 mg, 7.97 mmol), followed by biacetyl (1.05 equiv., 0.14 mL, 1.61 mmol). The mixture was stirred at 85° C. for 20 h. LCMS showed that desired product was formed (82%). The reaction mixture was combined with a second batch and filtered. The filtrate was concentrated and the filter cake was washed by DCM (20 mL). The resulting crude was purified by flash chromatography (0-67% EtOAc in PE) to give 2-bromo-6,7-dimethyl-4-(3-(trifluoromethyl) bicyclo[1.1.1]pentan-1-yl) pteridine (Int-A14, 520 mg, 1.39 mmol, 91% yield) as a solid. LCMS: [M+H]⁺=373.2; purity=82% (220 nm); Ret. time=0.612 min. ¹H NMR (400 MHz, DMSO-d₆) δ 2.76 (d, J=2.1 Hz, 6H), 2.64 (s, 6H).

An analogous procedure was followed to obtain the following intermediates.

| Structure | Starting Material | Characterization |
|---|---|---|
| 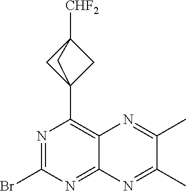 [SMILES: Cc1nc2nc(Br)nc(C34CC(C(F)F)(C3)C4)c2nc1C] | 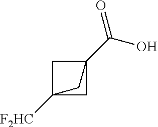 [SMILES: O=C(O)C12CC(C(F)F)(C1)C2] | The residue was purified by silica gel column chromatography (0-80% EtOAc in PE) (TLC: EtOAc, R_{f} = 0.6) to give 2-bromo-4-[3-(difluoromethyl)-1- bicyclo[1.1.1]pentanyl]-6, 7-dimethyl-pteridine (Int- A15, 500 mg, 1.41 mmol, 72% yield) as a solid. LCMS: Ret. Time = 0.570 min, [M + H]⁺ = 357.0 , ESI⁺. ¹H NMR (400 MHz, CDCl₃) δ 2.58 (s, 6H), 2.80 (d, J = 15.0 Hz, 6H), 5.67-6.09 (m, 1H). |
| Int-A15 | | |
| 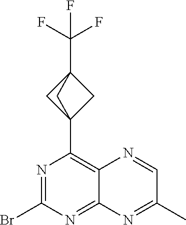 [SMILES: Cc1cnc2c(C34CC(C(F)(F)F)(C3)C4)nc(Br)nc2n1] | 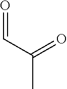 | Modifications: The last step was ran at 25° C. for 2 hours. The crude product was purified by silica gel column chromatography (0-50% EtOAc in PE) (TLC, 50% EtOAc in petroleum ether, desired product R_{f} = 0.5) to give 2-bromo-7-methyl-4-(3- (trifluoromethyl)bicyclo[1.1.1]pentan-1-yl) pteridine (Int-A50, 600 mg, 1.67 mmol, 60% yield) as a solid. LCMS: [M + H]⁺ = 359.0; purity = 97% (220 nm); Ret. time = 0.584 min. ¹H NMR (400 MHz, CDCl₃) δ 8.86 (s, 1H), 2.89 (s, 3H), 2.68 (s, 6H). |
| Int-A50 | | |
| 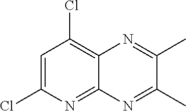 | 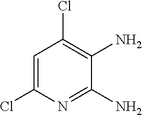 | Modification: The reaction was ran at 20° C. for 12 hours. The residue was purified by silica gel column chromatography (0-100% EtOAc in PE) (TLC, 25% EtOAc in PE, new product R_{f} = 0.6) to give 6,8-dichloro- 2,3-dimethyl-pyrido[2,3-b]pyrazine (Int-B21, 2.2 g, 9.65 mmol, 101% yield) as a solid. LCMS: Ret. time = 0.496 min; [M + H]⁺ = 227.9. ¹H NMR (400 MHz, CDCl₃) δ |
| | | 7.73 (s, 1H), 2.83 (s, 6H). |
| Int-B21 | | |
| 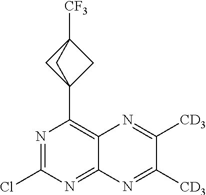 [SMILES: [2H]C([2H])([2H])c1nc2nc(Cl)nc(C34CC(C(F)(F)F)(C3)C4)c2nc1C([2H])([2H])[2H]] | 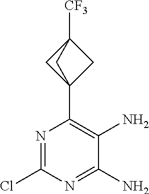 [SMILES: Nc1nc(Cl)nc(C23CC(C(F)(F)F)(C2)C3)c1N] | Modification: The reaction was ran at 80° C. for 16 hours, then cooled, filtered through Celite, and concentrated under reduced pressure to yield 2-chloro- 6,7-bis(methyl-d₃)-4-(3- (trifluoromethyl)bicyclo[1.1.1]pentan-1-yl)pteridine (Int-C13, 298 mg, 0.89 μmol, 99%) as a solid. ESI-MS: [M + H]⁺ = 279.1. ¹H NMR (CDCl₃, 400 MHz): δ 2.67 (s, 6H). ¹⁹F NMR (CDCl₃, 376 MHz): δ −73.1 (s, 3F). |
| Int-C13 | | |
| | 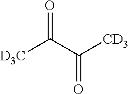 [SMILES: [2H]C([2H])([2H])C(=O)C(=O)C([2H])([2H])[2H]] | |

### Method Int 14. Intermediate Int-A16:2-benzyloxy-5-[(2R,4S,6R)-4-bromo-6-methyl-tetrahydropyran-2-yl]pyridine

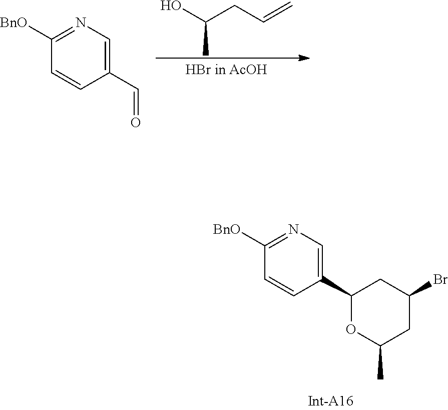

A mixture of 6-benzyloxypyridine-3-carbaldehyde (1 equiv., 10 g, 46.9 mmol) and (2R)-pent-4-en-2-ol (1 equiv., 4.8 mL, 46.9 mmol) in DCM (250 mL) was added dropwise HBr (in AcOH)(3 equiv., 25 mL, 141 mmol) at −20° C. under N₂ atmosphere. Then, the mixture was stirred at −20° C. for 4 h under N₂ atmosphere. LCMS showed desired product (48%). The reaction mixture was poured into NaHCO₃ (aq.) slowly to adjust the pH ≥7, extracted with EtOAc (300 mL×2), the organics were washed with 200 mL saturated brine solution. The combined organic layers were dried over Na₂SO₄, filtered, and concentrated under reduced pressure to give a crude residue. The crude was purified by Prep-HPLC (0.5% FA condition) and lyophilized to give 2-benzyloxy-5-[(2R,4S,6R)-4-bromo-6-methyl-tetrahydropyran-2-yl]pyridine (Int-A16, 5.4 g, 14.9 mmol, 32% yield) as an oil. LCMS: [M+H+2]⁺=364.0, purity=100% (220 nm), Ret. time=0.656 min. ¹H NMR (400 MHz, CDCl₃) δ 1.29 (dd, J=6.17, 2.6 Hz, 3H), 1.76-1.90 (m, 1H), 1.95-2.11 (m, 2H), 2.12-2.22 (m, 1H), 2.27-2.47 (m, 1H), 3.59-3.71 (m, 1H), 4.17-4.41 (m, 2H), 4.77-4.99 (m, 1H), 5.38 (s, 2H), 6.81 (dd, J=8.5, 3.6 Hz, 1H), 7.28-7.42 (m, 3H), 7.43-7.51 (m, 2H), 7.63 (dd, J=8.6, 1.9 Hz, 1H), 8.14 (dd, J=13.75, 1.9 Hz, 1H).

### Method Int 15. Intermediate Int-A17: bromo-[(2R,4S,6R)-2-methyl-6-(6-phenoxy-3-pyridyl)tetrahydropyran-4-yl]zinc

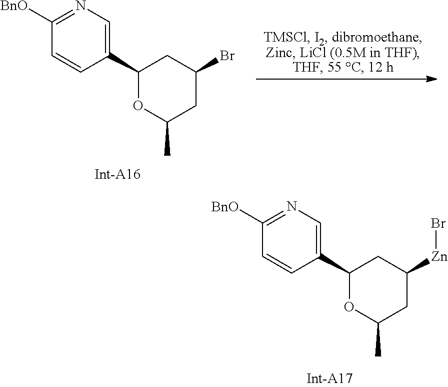

The synthesis was performed at a 2 g scale but 5 batches of 400 mg were performed in parallel.

Zinc (5.54 equiv., 2.0 g, 30.6 mmol) was suspended in LiCl (0.5 M in THF)(1 equiv., 20 mL, 5.52 mmol), 1,2-dibromoethane (0.05 equiv., 0.024 mL, 0.28 mmol) was added and the suspension was stirred at 55° C. for 20 min, cooled down, then TMSCl (0.05 equiv., mg, 0.276 mmol) was introduced and the mixture was stirred at 55° C. for additional 20 min. The mixture was cooled down, then iodine (0.02 equiv., 28 mg, 0.11 mmol) in THF (1 mL) was introduced and the reaction was stirred at 55° C. for another 20 min, 2-benzyloxy-5-[(2R,4S,6R)-4-bromo-6-methyl-tetrahydropyran-2-yl]pyridine (Int-A16, 1 equiv., 200 mg, 0.55 mmol) in THF (20 mL) was added to the warm suspension of activated zinc. The reaction mixture was stirred at 55° C. for 12 h, and the mixture was used in the next step directly.

An analogous procedure was followed to obtain the following intermediates.

| Structure | Starting Material | Characterization |
|---|---|---|
| 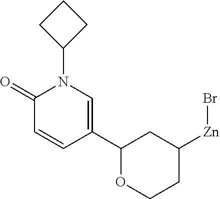 | 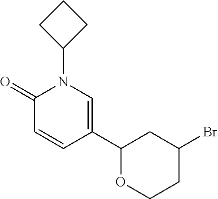 [SMILES: O=c1ccc(C2CC(Br)CCO2)cn1C1CCC1] | The procedure afforded a mixture of bromo-[2-(1- cyclobutyl-6-oxo-3-pyridyl)tetrahydropyran-4- yl]zinc (Int-F15) that was used directly in the next synthetic step. |
| Int-F15 | Int-F14 | |

### Method Int 16. Intermediate Int-A18:2-[(2R,6R)-2-(6-benzyloxy-3-pyridyl)-6-methyl-tetrahydropyran-4-yl]-6,7-dimethyl-4-[3-(trifluoromethyl)-1-bicyclo[1.1.1]-pentanyl]pteridine

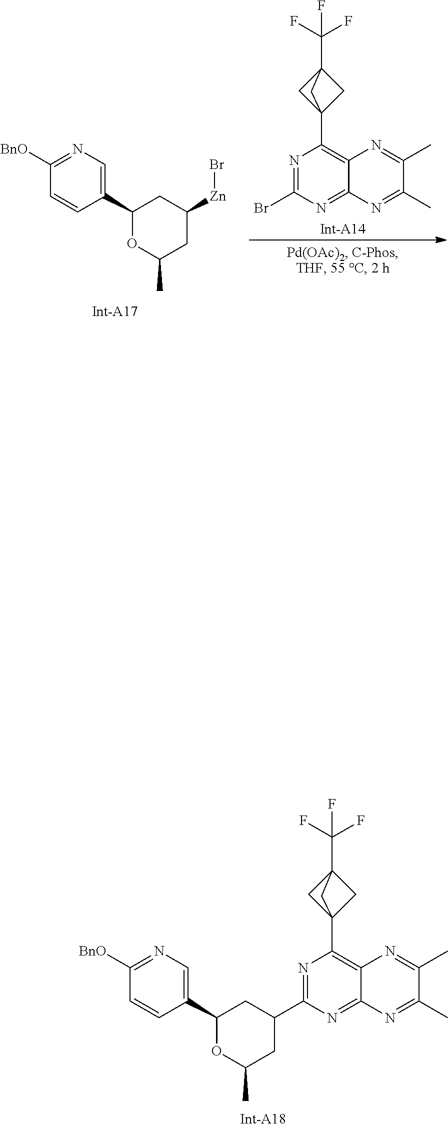

To a mixture of 2-bromo-6,7-dimethyl-4-[3-(trifluoromethyl)-1-bicyclo[1.1.1]-pentanyl]pteridine (Int-A14, 1 equiv., 100 mg, 0.268 mmol), C-Phos (0.2 equiv., 23 mg, 0.0536 mmol) and Pd(OAc)₂ (0.1 equiv., 6.0 mg, 0.0268 mmol), purged with N₂ for 3 times, THF (0.3 mL) was added as a solvent. Then, bromo-[(2R,4S,6R)-2-methyl-6-(6-phenoxy-3-pyridyl)tetrahydropyran-4-yl]zinc (Int-A17, 1.2 equiv., 5.0 mL, 0.322 mmol) was added and the mixture was stirred at 55° C. for 2 h. LCMS showed the trace amount of desired product. The reaction mixture (combined with other 5 batches) was quenched by 100 mL H₂O, extracted with EtOAc (100 mL×3), the organics were washed with 50 mL saturated brine solution. The combined organic layers were dried over Na₂SO₄, filtered, and concentrated under reduced pressure to give a crude residue. The crude was purified by silica gel column chromatography (0-50% PE in EtOAc)(TLC, 33% EtOAc in PE, desired product Rr-0.6) to give 2-[(2R,6R)-2-(6-benzyloxy-3-pyridyl)-6-methyl-tetrahydropyran-4-yl]-6,7-dimethyl-4-[3-(trifluoromethyl)-1-bicyclo[1.1.1]-pentanyl]pteridine (Int-A18, 700 mg, 1.11 mmol, 413% yield) as an oil. LCMS: [M+H]⁺=576.3, purity=91% (220 nm), Ret. time=0.729 min.

### Method Int 17. Intermediate Int-A20:2-chloro-4-(4,4-difluorocyclohex-1-en-1-yl)-6,7-dimethylpteridine

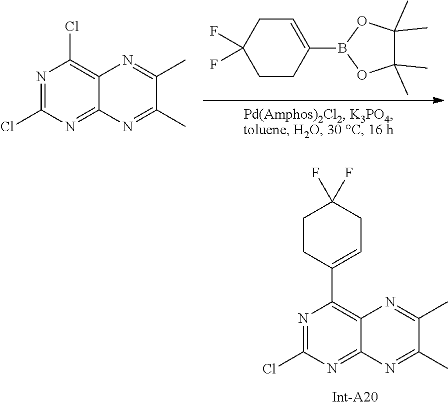

A mixture of 2,4-dichloro-6,7-dimethyl-pteridine (1 equiv., 1600 mg, 6.99 mmol), 2-(4,4-difluorocyclohexen-1-yl)-4,4,5,5-tetramethyl-1,3,2-dioxaborolane (1 equiv., 1705 mg, 6.99 mmol), K₃PO₄ (3 equiv., 4448 mg, 21.0 mmol) and Pd(Amphos)₂Cl₂ (0.1 equiv., 495 mg, 0.699 mmol) in toluene (50 mL) and water (5 mL) was degassed and purged with N₂ for 3 times, and then the mixture was stirred at 30° C. for 1 h under N₂ atmosphere. The reaction mixture was partitioned between EtOAc (2×100 mL) and water (80 mL) and extracted. The combined organic layers were dried over Na₂SO₄, filtered, and concentrated under reduced pressure to give a residue. The crude product was purified by silica gel column chromatography (0-100% EtOAc in PE)(TLC, 25% EtOAc in PE, desired product R=0.5) to give 2-chloro-4-(4,4-difluorocyclohex-1-en-1-yl)-6,7-dimethylpteridine (Int-A20, 1050 mg, 3.38 mmol, 48% yield) as an oil. LCMS: [M+H]⁺=311.1; purity=89% (220 nm); Ret. time=0.549 min. ¹H NMR (400 MHz, CDCl₃) δ 7.44-7.39 (m, 1H), 3.15-3.08 (m, 2H), 3.00-2.89 (m, 2H), 2.84-2.81 (m, 3H), 2.79 (s, 3H), 2.31-2.19 (m, 2H).

### Method Int 18. Intermediate Int-A21:2-chloro-4-(4,4-difluorocyclohexyl)-6,7-dimethyl-5,6,7,8-tetrahydropteridine

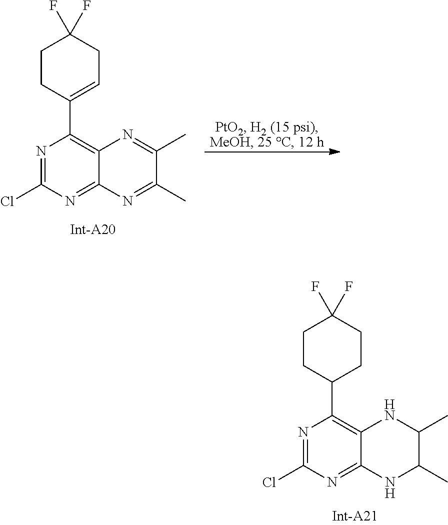 [SMILES: Cc1nc2nc(Cl)nc(C3=CCC(F)(F)CC3)c2nc1C ; "PtO2, H2 (15 psi),MeOH, 25 °C, 12 h" ; CC1Nc2nc(Cl)nc(C3CCC(F)(F)CC3)c2NC1C]

To a solution of 2-chloro-4-(4,4-difluorocyclohexen-1-yl)-6,7-dimethyl-pteridine (Int-A20, 1 equiv., 1050 mg, 3.38 mmol) in MeOH (20 mL) was added PtO₂ (1 equiv., 767 mg, 3.38 mmol) under N₂ atmosphere. The suspension was degassed and purged with H₂ for 3 times. The mixture was stirred under H₂ (15 psi) at 30° C. for 12 h. LCMS showed 39% of desired product. The reaction mixture was filtered and concentrated under reduced pressure to give a residue. The residue in MeOH (20 mL) was added PtO₂ (1 equiv., 767 mg, 3.38 mmol) under N₂ atmosphere. The suspension was degassed and purged with H₂ for 3 times. The mixture was stirred under H₂ (15 psi) at 25° C. for 12 h. LCMS showed 59% of desired product. The reaction mixture was filtered and concentrated under reduced pressure to give 1.0 g crude product (Int-A21).

### Method Int 19. Intermediate Int-A22:2-chloro-4-(4,4-difluorocyclohexyl)-6,7-dimethylpteridine

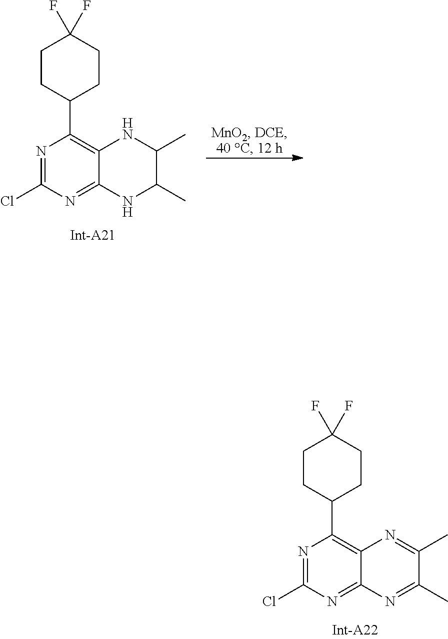 [SMILES: CC1Nc2nc(Cl)nc(C3CCC(F)(F)CC3)c2NC1C ; "MnO2, DCE,40 °C, 12 h" ; Cc1nc2nc(Cl)nc(C3CCC(F)(F)CC3)c2nc1C]

A mixture of 2-chloro-4-(4,4-difluorocyclohexyl)-6,7-dimethyl-5,6,7,8-tetrahydropteridine (Int-A21, 1 equiv., 1.0 g, 3.16 mmol) in DCE (50 mL) was added MnO₂ (10 equiv., 2.74 g, 31.6 mmol), then stirred at 40° C. for 12 h. LCMS showed a peak with desired mass was found. The reaction mixture was filtered and concentrated under reduced pressure to give a residue (about 900 mg). The residue in DCE (50 mL) was added MnO₂ (10 equiv., 2.74 g, 31.6 mmol), then stirred at 40° C. for 12 h. LCMS showed 73% of desired product. The reaction mixture was filtered and concentrated under reduced pressure to give a residue (about 660 mg). The crude product was purified by silica gel column chromatography (0-100% EtOAc in PE)(TLC, 25% EtOAc in PE, desired product R_{f}=0.4) to give 300 mg (54% purity) and 2-chloro-4-(4,4-difluorocyclohexyl)-6,7-dimethylpteridine (Int-A22, 250 mg, 0.799 mmol, 25% yield) as a solid. LCMS: [M+H]⁺=313.2; purity=96% (220 nm); Ret. time=0.553 min. ¹H NMR (400 MHz, CDCl₃) δ 4.20-4.09 (m, 1H), 2.84 (s, 3H), 2.80 (s, 3H), 2.37-2.26 (m, 2H), 2.25-2.12 (m, 2H), 2.09-1.93 (m, 4H).

### Method Int 20. Intermediate Int-A24:3-(1-hydroxy-1-methyl-ethyl) bicyclo[1.1.1]pentane-1-carboxylic acid

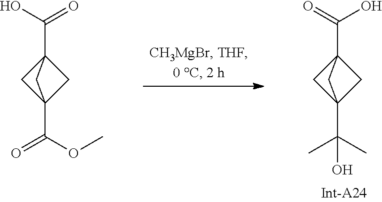

To a solution of 3-methoxycarbonylbicyclo[1.1.1]pentane-1-carboxylic acid (1 equiv., 5.0 g, 29.4 mmol) in THF (100 mL) was added methylmagnesium bromide (4 equiv., 39 mL, 118 mmol) at 0° C., the solution was stirred at 0° C. for 2 h. TLC showed starting material was consumed completely and two new spots found. The solution was added water (10 mL) and extracted with EtOAc (10 mL×3) and the organic phase was concentrated under reduced pressure to give a crude. The crude was purified by flash column (100% EtOAc in PE, R_{f}=0.3) and concentrated under reduced pressure to give two batches of product. Batch 1:3-(1-hydroxy-1-methyl-ethyl) bicyclo[1.1.1]pentane-1-carboxylic acid (Int-A24, 2.0 g, 12.3 mmol, 42% yield) as a solid and batch 2:3-(1-hydroxy-1-methyl-ethyl) bicyclo[1.1.1]pentane-1-carboxylic acid (Int-A24, 2.1 g, 12.3 mmol, 40% yield) as a solid.

(Batch 1): ¹H NMR (400 MHz, CDCl₃) δ 1.99 (s, 6H), 1.19-1.17 (m, 6H).

(Batch 2): ¹H NMR (400 MHz, CDCl₃) δ 1.99-1.97 (m, 3H), 1.94-1.89 (m, 3H), 1.22-1.15 (m, 6H).

### Method Int 21. Intermediate Int-A25:3-(1-hydroxy-1-methyl-ethyl)-N-methoxy-N-methyl-bicyclo[1.1.1]pentane-1-carboxamide

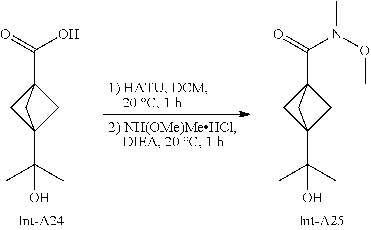

A solution of 3-(1-hydroxy-1-methyl-ethyl) bicyclo[1.1.1]pentane-1-carboxylic acid (Int-A24, 1 equiv., 2.0 g, 11.8 mmol) in DCM (40 mL) was purged under N₂ and stirred at 20° C. The solution was added HATU (1.5 equiv., 6702 mg, 17.6 mmol) and stirred for 1 h under N₂ at 20° C. N,N-diisopropylethylamine (2 equiv., 4.1 mL, 23.5 mmol) and N,O-dimethylhydroxylamine hydrochloride (1.2 equiv., 1375 mg, 14.1 mmol) were added and the reaction mixture was continued to stirred at 20° C. for 12 h. LCMS showed starting material was consumed completely and a peak with desired mass was detected. The reaction was combined with a second batch. The final mixture was concentrated under reduced pressure to give a crude. The crude was purified by flash column (67% EtOAc in PE, R_{f}=0.3) to give 3-(1-hydroxy-1-methyl-ethyl)-N-methoxy-N-methyl-bicyclo[1.1.1]pentane-1-carboxamide (Int-A25, 2.10 g, 7.68 mmol, 65% yield) as a solid. LCMS: [M+H]⁺=214.0; purity=79% (UV 220 nm); Ret. time=0.384 min. ¹H NMR (400 MHz, CDCl₃) δ 3.67 (s, 3H), 3.18 (s, 3H), 2.80-2.79 (m, 1H), 2.03-1.98 (m, 6H), 1.18 (s, 6H).

### Method Int 22. Intermediate Int-A26: methyl-3-(1-fluoro-1-methyl-ethyl) bicyclo[1.1.1]pentane-1-carboxylate

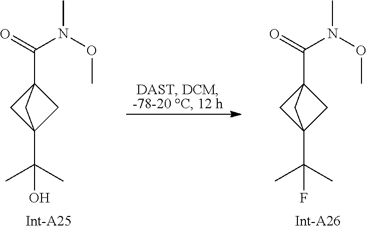

A mixture of methyl 3-(1-hydroxy-1-methyl-ethyl) bicyclo[1.1.1]pentane-1-carboxylate (Int-A25, 1 equiv., 170 mg, 0.923 mmol) in DCM (4 mL) was cooled to-78° C. Then DAST (3 equiv., 0.26 mL, 2.77 mmol) was added slowly at −78° C. The mixture was warmed to 20° C. and stirred for 12 h. LCMS showed starting material consumed completely and a peak with desired mass was detected. The mixture was cooled to-78° C. and added water slowly. Then the solution was added 20 mL water and extracted with EtOAc (20 mL×3). The combined organic layer was added silica gel and concentrated under reduced process to give the crude. The crude was purified by flash column (17% EtOAc in PE, R_{f}=0.3) to afford methyl-3-(1-fluoro-1-methyl-ethyl) bicyclo[1.1.1]pentane-1-carboxylate (Int-A26, 50 mg, 0.268 mmol, 29% yield) as a liquid. LCMS: [M+H]⁺=216.2; purity=86% (UV 220 nm); Ret. time =0.453 min. ¹H NMR (400 MHz, CDCl₃) δ 3.67 (s, 3H), 3.19 (s, 3H), 2.06 (s, 6H), 1.36-1.32 (m, 3H), 1.31-1.26 (m, 3H).

### Method Int 23. Intermediate Int-A27: (3-chloro-5,6-dimethyl-pyrazin-2-yl)-[3-(1-fluoro-1-methyl-ethyl)-1-bicyclo[1.1.1]pentanyl]methanone

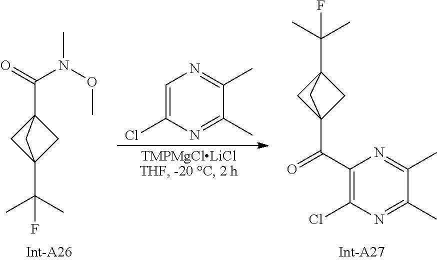

To a solution of 3-(1-fluoro-1-methyl-ethyl)-N-methoxy-N-methyl-bicyclo[1.1.1]pentane-1-carboxamide (Int-A26, 1 equiv., 200 mg, 0.93 mmol) in THF (6 mL) was added 5-chloro-2,3-dimethylpyrazine (1.5 equiv., 199 mg, 1.39 mmol). The flask was capped and purged under N₂. The solution was cooled to-20° C. TMPMgCl·LiCl (1.5 equiv., 1.4 mL, 1.39 mmol) was added dropwise over 2 min, and the reaction mixture was stirred at −20° C. for 1 h. LCMS showed 58% starting material (SM₁) and 14% SM₂ remained and a peak with desired MS was detected. TMPMgCl·LiCl (0.75 equiv., 0.70 mL, 0.697 mmol) was added at 0° C. and the mixture was stirred at 0° C. for 1 h. LCMS showed 56% SM₁ remained and a peak with desired mass was detected. The reaction mixture was diluted with water 5 mL and extracted with solvent EtOAc (10 mL×3). The combined organic layers were dried over Na₂SO₄, filtered, and concentrated under reduced pressure to give a residue. The residue was purified by reversed-phase chromatography (40% [water (FA)-ACN]) to give (3-chloro-5,6-dimethyl-pyrazin-2-yl)-[3-(1-fluoro-1-methyl-ethyl)-1-bicyclo[1.1.1]pentanyl]methanone (Int-A27, 140 mg, 0.406 mmol, 44% yield) as a solid. LCMS: [M+H]⁺=279.2; purity=86% (220 nm); Ret. time=0.590 min. ¹H NMR (400 MHz, CDCl₃) δ 2.64-2.50 (m, 6H), 2.22-2.13 (m, 6H), 1.37-1.32 (m, 3H), 1.31-1.28 (m, 3H).

### Method Int 24. Intermediate Int-A28: methyl-3-(hydroxymethyl) bicyclo[1.1.1]pentane-1-carboxylate

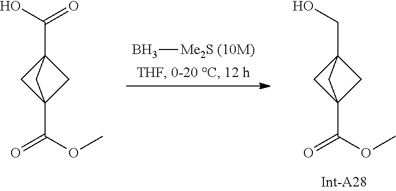

A mixture of 3-methoxycarbonylbicyclo[1.1.1]pentane-1-carboxylic acid (1 equiv., 20 g, 118 mmol) in THF (800 mL) was cooled to 0° C. BH₃-Me₂S (1.2 equiv., 14 mL, 141 mmol) was added slowly at 0° C. The mixture was then stirred at 20° C. for 12 h. TLC showed a new spot found. MeOH was added slowly until gas was produced. The mixture was concentrated under reduced pressure to give a crude residue, which was purified by flash column (50% EtOAc in PE; phosphomolybdic acid; R_{f}=0.3) to give methyl-3-(hydroxymethyl) bicyclo[1.1.1]pentane-1-carboxylate (Int-A28, 18.3 g, 117 mmol, 99.7% yield) as an oil. ¹H NMR (400 MHz, CDCl₃) δ 3.68 (s, 3H), 3.64 (s, 2H), 2.00 (s, 6H).

### Method Int 25. Intermediate Int-A29: methyl 3-formylbicyclo[1.1.1]pentane-1-carboxylate

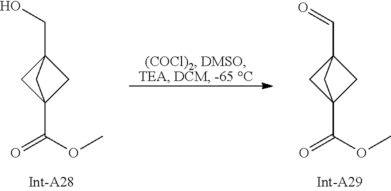

A mixture of oxalyl chloride (1.3 equiv., 7.1 mL, 83.2 mmol) in DCM (160 mL) was cooled to-65° C., then DMSO (2.6 equiv., 13.01 g, 166 mmol) in DCM (26 mL) was added slowly below −60° C. The mixture was stirred at −65° C. for 0.5 h. Then methyl 3-(hydroxymethyl) bicyclo[1.1.1]pentane-1-carboxylate (Int-A28, 1 equiv., 10 g, 64.0 mmol) in DCM (180 mL) was added slowly. The mixture was stirred at −65° C. for 0.5 h. The mixture was added 50 mL TEA at −65° C. The mixture was then warmed to 20° C. and filtered. The filter cake was washed with 200 mL EtOAc and the filtrate was concentrated under reduced pressure to give a crude. The crude was purified by flash column chromatography (25% EtOAc in PE; (2,4-dinitrophenyl) hydrazine; R_{f}=0.2) to give methyl 3-formylbicyclo[1.1.1]pentane-1-carboxylate (Int-A29, 8.6 g, 55.8 mmol, 87% yield) as an oil. ¹H NMR (400 MHz, CDCl₃) δ 9.60 (s, 1H), 3.70 (s, 3H), 2.32 (s, 6H).

### Method Int 26. Intermediate Int-A30: methyl-3-(2,2-difluorovinyl) bicyclo-[1.1.1]pentane-1-carboxylate

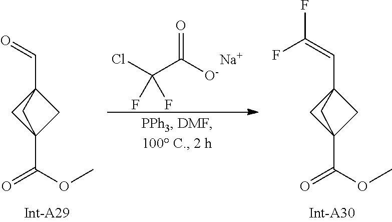

To a mixture of methyl 3-formylbicyclo[1.1.1]pentane-1-carboxylate (Int-A29, 1 equiv., 5 g, 32.4 mmol) in DMF (100 mL) was added PPh₃ (3 equiv., 26 g, 97.3 mmol) and sodium chlorodifluoroacetate (3 equiv., 14.8 g, 97.3 mmol) at 20° C., and the mixture was degassed with N₂ for 3 times. The mixture was stirred at 100° C. for 1 h. TLC showed the starting material was consumed completely. TLC showed a new spot was detected. The mixture was added to water (100 mL) and extracted with EtOAc (100 mL×3). To the combined organic layers were added silica gel, and the mixture was concentrated under reduced pressure to give the crude which was purified by silica gel column chromatography (25% EtOAc in PE, R_{f}=0.2) to afford methyl-3-(2,2-difluorovinyl) bicyclo-[1.1.1]pentane-1-carboxylate (Int-A30, 1.4 g, 7.44 mmol, 23% yield) as an oil. ¹H NMR (400 MHz, CDCl₃) δ 4.34-4.22 (m, 1H), 3.67 (s, 3H), 2.17 (s, 6H).

### Method Int 27. Intermediate Int-A31: methyl-3-(2,2-difluoroethyl) bicyclo[1.1.1]pentane-1-carboxylate

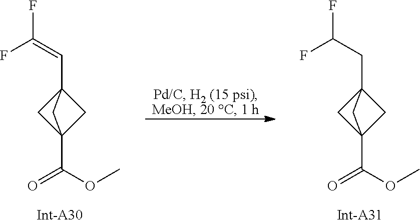

To a mixture of methyl 3-(2,2-difluorovinyl) bicyclo[1.1.1]pentane-1-carboxylate (Int-A30, 1 equiv., 700 mg, 3.72 mmol) in MeOH (16 mL) was added Palladium on carbon (1 equiv., 396 mg, 3.72 mmol) at 20° C. under N₂. The mixture was degassed with H₂ for 3 times and stirred at 20° C. for 2 h under H₂ (15 psi) atmosphere. TLC showed the starting material was consumed completely and a new spot was detected. The mixture degassed with N₂ for three times. The mixture was filtered across Celite and the filtrate was concentrated under reduced pressure to give methyl-3-(2,2-difluoroethyl) bicyclo[1.1.1]pentane-1-carboxylate (Int-A31, 560 mg, 2.94 mmol, 79% yield) as an oil. 1H NMR (400 MHz, CDCl₃) δ 5.80 (tt, J=4.5, 56.2 Hz, 1H), 3.67 (s, 3H), 2.11-1.99 (m, 8H).

### Method Int 28. Intermediate Int-A32:3-(2,2-difluoroethyl) bicyclo[1.1.1]pentane-1-carboxylic acid

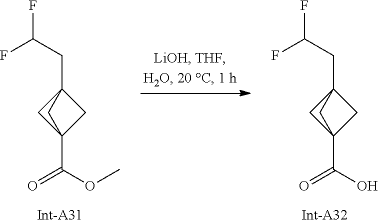

To a mixture of methyl 3-(2,2-difluoroethyl) bicyclo[1.1.1]pentane-1-carboxylate (Int-A31, 1 equiv., 560 mg, 2.94 mmol) in THF (8.4 mL) and water (2.8 mL) was added LiOH (2 equiv., 141 mg, 5.89 mmol) at 25° C. The mixture was stirred at 25° C. for 1 h. TLC showed the starting material was consumed completely and a new spot was detected. The mixture was added water (20 mL) and extracted with EtOAc (20 mL×3). The combined organic layers were dried over Na₂SO₄, filtered, and concentrated under reduced pressure to give 3-(2,2-difluoroethyl) bicyclo[1.1.1]pentane-1-carboxylic acid (Int-A32, 600 mg, 3.41 mmol, 116% yield). ¹H NMR (400 MHz, CDCl₃) δ 5.91-5.56 (m, 1H), 2.02 (s, 6H), 2.00-1.93 (m, 2H).

### Method Int 29. Intermediate Int-A33:3-(2,2-difluoroethyl)-N-methoxy-N-methyl-bicyclo[1.1.1]pentane-1-carboxamide

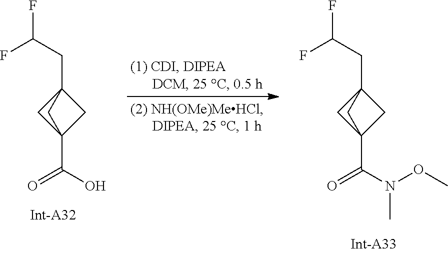

To a solution of 3-(2,2-difluoroethyl) bicyclo[1.1.1]pentane-1-carboxylic acid (Int-A32, 1 equiv., 660 mg, 3.75 mmol) in DCM (30 mL) was added CDI (1.3 equiv., 790 mg, 4.87 mmol) and N,N-diisopropylethylamine (1.3 equiv., 0.85 mL, 4.87 mmol). The mixture was stirred at 25° C. for 0.5 h. The mixture was added N,N-diisopropylethylamine (1.5 equiv., 0.98 mL, 5.62 mmol) and N,O-dimethylhydroxylamine hydrochloride (1.3 equiv., 475 mg, 4.87 mmol) at 25° C. The mixture was stirred at 25° C. for 12 h. LCMS showed the starting material was consumed completely and a major peak with desired mass was detected. The mixture was added water (50 mL) and extracted with EtOAc (50 mL×3). The combined organic layers were concentrated under reduced pressure to give a crude. The crude was purified by reversed-phase chromatography (60% [water (FA)-ACN]), the solution was removed ACN and the remainder was extracted with EtOAc (3×50 mL). The combined organic phase was concentrated under reduced pressure to give 3-(2,2-difluoroethyl)-N-methoxy-N-methyl-bicyclo[1.1.1]pentane-1-carboxamide (Int-A33, 600 mg, 2.74 mmol, 73% yield) as an oil. LCMS: [M+H]⁺=220.2; purity=82% (UV 220 nm); Ret. time=0.456 min. ¹H NMR (400 MHz, CDCl₃) δ 5.81 (tt, J=4.6, 56.3 Hz, 1H), 3.67 (s, 3H), 3.18 (s, 3H), 2.13-2.10 (m, 6H), 2.10-2.00 (m, 2H).

An analogous method was followed to obtain the following intermediates.

| Structure | Starting Material | Characterization |
|---|---|---|
| 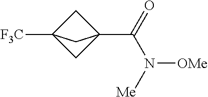 [SMILES: CON(C)C(=O)C12CC(C(F)(F)F)(C1)C2] | 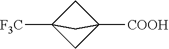 [SMILES: O=C(O)C12CC(C(F)(F)F)(C1)C2] | Modification: The reaction was started from 3- (trifluoromethyl)bicyclo[1.1.1]pentane-1- carboxylic acid to obtain N-methoxy-N-methyl-3- (trifluoromethyl)bicyclo[1.1.1]pentane-1- carboxamide (Int-E4, 3.09 g, 13.8 mmol, 92% yield) as an oil, which slowly crystallizes into a |
| | | solid. ¹H NMR (CDCl₃, 400 MHz): δ 3.68 (3H, s), |
| Int-E4 | | 3.19 (3H, s), 2.29 (6H, s). ¹⁹F NMR (CDCl₃, 376 |
| | | MHz): δ −73.2 (3F, s). LCMS: [M + H]⁺ = 224.2. |

### Method Int 30. Intermediate Int-A34: (3-chloro-5,6-dimethyl-pyrazin-2-yl)-[3-(2,2-difluoroethyl)-1-bicyclo[1.1.1]pentanyl]methanone

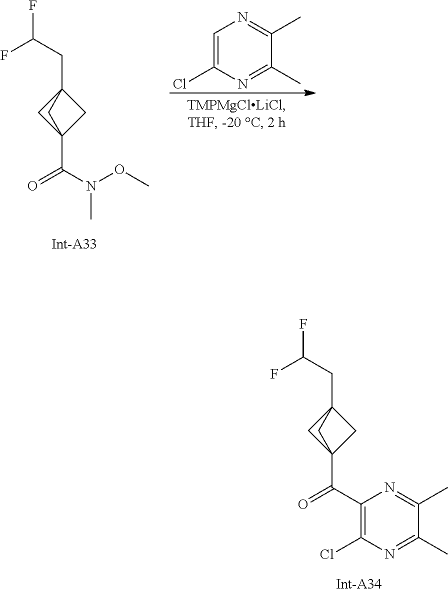

To a mixture of 3-(2,2-difluoroethyl)-N-methoxy-N-methyl-bicyclo[1.1.1]pentane-1-carboxamide (Int-A33, 1 equiv., 300 mg, 1.4 mmol) and 5-chloro-2,3-dimethyl-pyrazine (1.5 equiv., 0.25 mL, 2.1 mmol) in THF (4.5 mL) was degassed with N₂ for 3 times. The mixture was cooled to-20° C. and added TMPMgCl·LiCl (2.2 equiv., 3.0 mL, 3 mmol) dropwise at −20° C. The mixture was stirred at-20° C. for 2 h. LCMS showed 38% starting material remained and a peak with desired mass was detected. The mixture was stirred at −20° C. for another 1 h. LCMS showed starting material consumed completely and a peak with desired mass was detected. The mixture was added into water (10 mL) and extracted with EtOAc (10 mL×3) and concentrated under reduce pressure to give a crude. The crude product was purified by reversed-phase flash chromatography (0.1% FA condition). The desired fractions were concentrated to remove CH₃CN, and adjusted to pH about 9 ~10 using Na₂CO₃ solid, followed by extracted with EtOAc (3×10 mL). The combined organic layers were dried over anhydrous Na₂SO₄, filtered, and concentrated under reduced pressure to give (3-chloro-5,6-dimethyl-pyrazin-2-yl)-[3-(2,2-difluoroethyl)-1-bicyclo[1.1.1]pentanyl]methanone (Int-A34, 190 mg, 0.632 mmol, 46% yield) as a solid. LCMS: [M+H]⁺=301.1; purity=55% (UV 220 nm); Ret. time=0.581 min. ¹H NMR (400 MHz, CDCl₃) δ 6.00-5.64 (m, 1H), 2.58 (s, 3H), 2.56 (s, 3H), 2.23 (s, 6H), 2.08 (dt, J=4.6, 17.6 Hz, 2H).

### Method Int 31. Intermediate Int-A35: prop-1-ynylcyclopropane

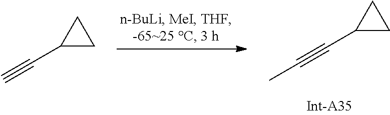

To a dried three-neck reaction flask was added THF (400 mL) and ethynylcyclopropane (1 equiv., 20 g, 303 mmol). The mixture was degassed with N₂ for 3 times and cooled to-65° C. n-BuLi (1.6 equiv., 194 mL, 484 mmol) was added slowly to the mixture below −60° C., after, the mixture was stirred at −78° C. for 1 hour. Then MeI (3 equiv., 57 mL, 908 mmol) was added slowly below −60° C., the reaction was stirred at −60° C. for 1 h and warmed to 20° C. for 1 h. The reaction mixture was poured into saturated NH₄Cl aqueous solution (200 mL) and then extracted with DCM (3×200 mL). The combined organic layers were dried over Na₂SO₄, filtered. Then most of solvent was evaporated (<25° C., 25 mmHg) to give a crude product prop-1-ynylcyclopropane (Int-A35, 53 g, 185 mmol, 61% yield) as a liquid. The crude product was used in the next step without further purification.

### Method Int 32. Intermediate Int-A36:1-cyclopropylpropane-1,2-dione

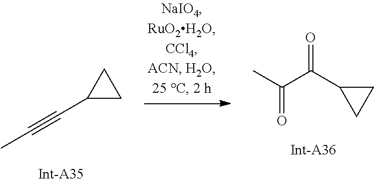

To a solution of prop-1-ynylcyclopropane (Int-A35, 1 equiv., 20.0 g, 62.4 mmol) in carbon tetrachloride (100 mL) and MeCN (100 mL) was added NaIO₄ (2.2 equiv., 29362 mg, 137 mmol)(dissolved in water (100 mL)). Then RuO₂·H₂O (0.022 equiv., 207 mg, 1.37 mmol) was added (NOTE: After adding RuO₂·H₂O, a lot of green precipitate formed). Then the reaction mixture was stirred vigorously under air for 2 h. TLC showed one new spot formed, which had fluorescence under 254 nm. The reaction mixture was filtered through a pad of Celite. The filter cake was washed with DCM (100 mL). The filtrates was diluted with water (100 mL) and then extracted with DCM (3×50 mL). The combined organic layers were washed with brine, dried over Na₂SO₄. Then the organic layers was combined with another batch and purified by distillation. Then crude product (9 g) was given (760 mmHg, b.p. >90° C.) as an oil. Then it was further purified by distillation under reduced pressure. Pure 1-cyclopropylpropane-1,2-dione (Int-A36, 1650 mg, 14.7 mmol, 24% yield)(20 mmHg, b.p. =56 ~60° C.) was given as an oil. ¹H NMR: (400 MHz, CDCl₃, 299 K) δ 2.70 (tt, J=4.9, 7.6 Hz, 1H), 2.29 (s, 3H), 1.10-1.00 (m, 4H).

### Method Int 33. Intermediate Int-A37:2-bromo-6-cyclopropyl-7-methyl-4-[3-(trifluoromethyl)-1-bicyclo[1.1.1]pentanyl]pteridine, Intermediate Int-A38:2-bromo-7-cyclopropyl-6-methyl-4-[3-(trifluoromethyl)-1-bicyclo[1.1.1]pentanyl]pteridine

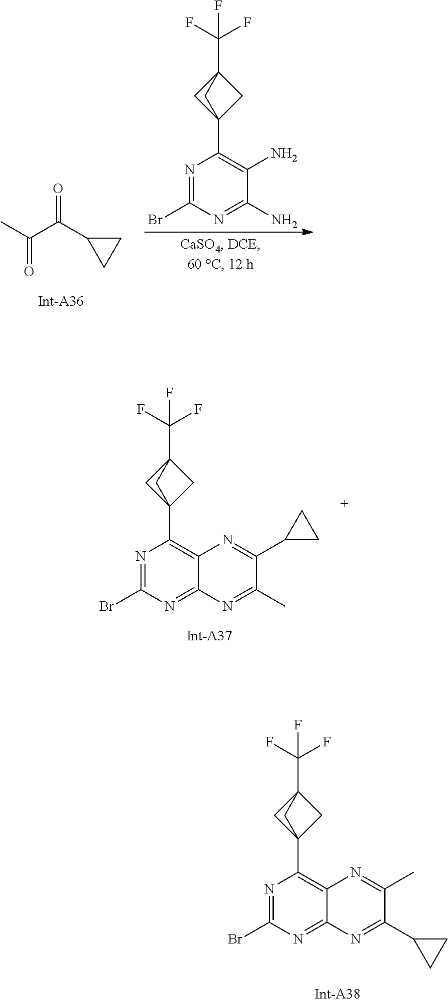

The mixture of 2-bromo-6-[3-(trifluoromethyl)-1-bicyclo[1.1.1]pentanyl]pyrimidine-4,5-diamine (1 equiv., 500 mg, 1.32 mmol), 1-cyclopropylpropane-1,2-dione (Int-A36, 1.6 equiv., 295 mg, 2.10 mmol) and CaSO₄ (6 equiv., 1074 mg, 7.89 mmol) in DCE (10 mL) was stirred at 60° C. for 12 h. LCMS showed 39% & 49% of products. The reaction mixture was combined with another batch to work up. The combined reaction mixtures were filtered through a pad of Celite. The filter cake was washed with EtOAc (20 mL). The filtrate was evaporated under reduced pressure to afford a residue (700 mg). The residue was purified by silica gel column chromatography (12% EtOAc in Petroleum ether, TLC, 25% EtOAc in PE, R_{f}=0.7)(50 mL/min) to give 2-bromo-6-cyclopropyl-7-methyl-4-[3-(trifluoromethyl)-1-bicyclo[1.1.1]pentanyl]pteridine (Int-A37, 325 mg, 0.81 mmol, 62% yield) as crystals and 2-bromo-7-cyclopropyl-6-methyl-4-[3-(trifluoromethyl)-1-bicyclo[1.1.1]pentanyl]pteridine (Int-A38, 280 mg, 0.70 mmol, 53% yield)(17% EtOAc in Petroleum ether, TLC, 25% EtOAc in PE, R_{f}=0.65) as a solid.

Int-A37: LCMS: [M+H]⁺=401.0; purity=99.5% (220 nm); Ret. time=0.644 min. 1H NMR (400 MHz, CDCl₃, 301 K) δ 2.91 (s, 3H), 2.66 (s, 6H), 2.36 (tt, J=4.6, 7.9 Hz, 1H), 1.56-1.50 (m, 2H), 1.35-1.29 (m, 2H).

Int-A38: LCMS: [M+H]⁺=401.0; purity=100% (220 nm); Ret. time=0.625 min. ¹H NMR (400 MHz, CDCl₃, 301 K) δ 2.96 (s, 3H), 2.62 (s, 6H), 2.44-2.35 (m, 1H), 1.36-1.24 (m, 4H).

### Method Int 34. Intermediate Int-A39: methyl-(Z)-3-(2-fluorovinyl) bicyclo[1.1.1]pentane-1-carboxylate

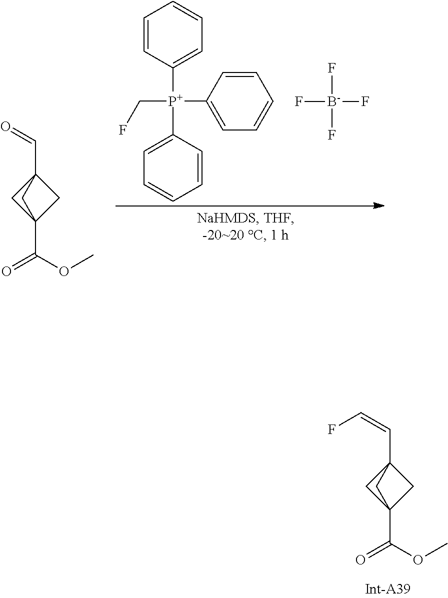 [SMILES: COC(=O)C12CC(C=O)(C1)C2 ; FC[P+](c1ccccc1)(c1ccccc1)c1ccccc1 ; F[B-](F)(F)F ; COC(=O)C12CC(/C=C\F)(C1)C2]

A mixture of fluoromethyl (triphenyl)phosphonium; tetrafluoroborate (1.5 equiv., 7.44 g, 19.5 mmol) in THF (75 mL) was degassed with N₂ for 3 times and cooled to-20° C. Then NaHMDS (1.5 equiv., 19 mL, 19.5 mmol) was added dropwise. The mixture was stirred at −20° C. for 15 min. Methyl 3-formylbicyclo[1.1.1]pentane-1-carboxylate (1 equiv., 2.0 g, 13.0 mmol) in THF (5 mL) was added at −20° C. The mixture was then stirred at 20° C. for 1 h. LCMS showed the reactant was consumed but no desired mass was detected and major of triphenylphosphine oxide. TLC showed one new point was detected. The mixture was diluted with 100 mL water and extracted with EtOAc (3×100 mL) and concentrated under reduced pressure to give a crude, which was purified by silica gel column chromatography (9% EtOAc in PE, KMnO₄, R_{f}=0.6) to afford methyl-(Z)-3-(2-fluorovinyl) bicyclo[1.1.1]pentane-1-carboxylate (Int-A39, 500 mg, 2.23 mmol, 17% yield) as a liquid. ¹H NMR (400 MHz, CDCl₃) δ 6.61-6.30 (m, 1H), 5.52-4.70 (m, 1H), 3.68 (s, 3H), 2.28-2.21 (m, 2H), 2.12-2.05 (m, 4H).

### Method Int 35. Intermediate Int-A40: methyl-3-(2-fluoroethyl) bicyclo[1.1.1]pentane-1-carboxylate

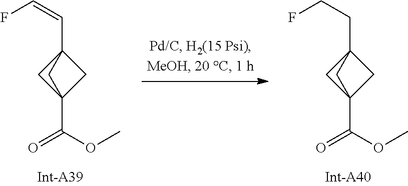

To a mixture of methyl 3-[(Z)-2-fluorovinyl]bicyclo[1.1.1]pentane-1-carboxylate (Int-A39, 1 equiv., 500 mg, 2.94 mmol) in MeOH (10 mL) was added palladium on carbon (1 equiv., 313 mg, 2.94 mmol) at 20° C. under N₂. The mixture was degassed with H₂ for 3 times and stirred at 20° C. for 2 h under H₂ (15 psi) atmosphere. TLC showed the starting material was consumed completely. The mixture degassed with N₂ for three times. The mixture was filtered through Celite and the filtrate was concentrated under reduced pressure to give methyl-3-(2-fluoroethyl) bicyclo[1.1.1]pentane-1-carboxylate (Int-A40, 350 mg, 2.03 mmol, 69% yield) as a liquid. ¹H NMR (400 MHz, CDCl₃) δ 4.59-4.37 (m, 2H), 3.67 (s, 3H), 2.05-2.00 (m, 6H), 1.90-1.85 (m, 2H).

### Method Int 36. Intermediate Int-A41:3-(2-fluoroethyl) bicyclo[1.1.1]pentane-1-carboxylic acid

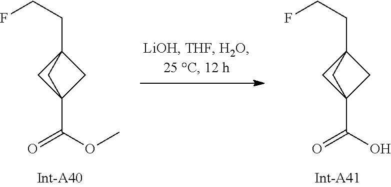

A mixture of methyl 3-(2-fluoroethyl) bicyclo[1.1.1]pentane-1-carboxylate (Int-A40, 1 equiv., 350 mg, 2.03 mmol) in THF (6 mL) and water (2 mL) was added LiOH (2 equiv., 98 mg, 4.07 mmol) at 20° C. The mixture was stirred at 20° C. for 12 h. TLC showed the starting material was consumed completed and one new spot found. The reaction mixture was partitioned between EtOAc (30 mL) and water (30 mL). The combined aqueous layers was added 1M HCl (1 mL) to adjust pH<7, partitioned between EtOAc (2×40 mL) and water (30 mL) and extracted. The combined organic layers were dried over Na₂SO₄, filtered, and concentrated under reduced pressure to give 3-(2-fluoroethyl) bicyclo[1.1.1]pentane-1-carboxylic acid (Int-A41, 270 mg, 1.71 mmol, 84% yield) as an oil. ¹H NMR (400 MHz, CDCl₃) δ 4.58-4.38 (m, 2H), 2.04 (s, 6H), 1.93-1.90 (m, 2H).

An analogous method was followed to obtain the following intermediates.

| Structure | Starting Material | Characterization |
|---|---|---|
| 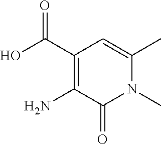 | 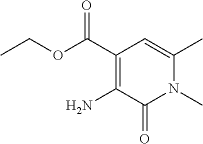 | Modification: The reaction was run THF/MeOH/H₂O solvent mixture (1:1:1 volumetric ratio) to obtain 3-amino-1,6-dimethyl-2-oxo- pyridine-4-carboxylic acid (7.50 g, 41.2 mmol, 96% yield) as a solid. LCMS: [M + H]⁺ = 183.0. ¹H NMR (400 MHz, DMSO-d₆) δ 6.30 (br s, 1H), 3.45 (br s, 3H), 2.23 (br s, 3H). |
| Int-C30 | Int-D29 | |

### Method Int 37. Intermediate Int-A42:3-(2-fluoroethyl)-N-methoxy-N-methylbicyclo[1.1.1]pentane-1-carboxamide

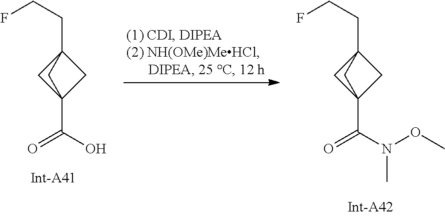

To a solution of 3-(2-fluoroethyl) bicyclo[1.1.1]pentane-1-carboxylic acid (Int-A41, 1 equiv., 240 mg, 1.52 mmol) in DCM (5 mL) was added CDI (1.3 equiv., 320 mg, 1.97 mmol) and N,N-diisopropylethylamine (1.3 equiv., 0.34 mL, 1.97 mmol), and the mixture was stirred at 25° C. for 0.5 h. The mixture was added N,N-diisopropylethylamine (1.5 equiv., 0.40 mL, 2.28 mmol) and N,O-dimethylhydroxylamine hydrochloride (1.3 equiv., 192 mg, 1.97 mmol) at 25° C. The mixture was stirred at 25° C. for 12 h. LCMS showed 57% of desired product. The reaction mixture was partitioned between EtOAc (2× 50 mL) and water (80 mL) and extracted. The combined organic layers were dried over Na₂SO₄, filtered, and concentrated under reduced pressure to give a residue. The crude residue was purified by reversed-phase HPLC (0.1% FA condition) to give 3-(2-fluoroethyl)-N-methoxy-N-methylbicyclo[1.1.1]pentane-1-carboxamide (Int-A42, 150 mg, 0.745 mmol, 49% yield) as an oil. LCMS: [M+H]⁺=202.2; purity=100% (220 nm); Ret. time=0.443 min. ¹H NMR (400 MHz, CDCl₃) δ 4.59-4.39 (m, 2H), 3.67 (s, 3H), 3.22-3.15 (m, 3H), 2.08-2.04 (m, 6H), 1.95-1.83 (m, 2H).

An analogous method was followed to obtain the following intermediates.

| | Starting | |
|---|---|---|
| Structure | Material | Characterization |
| 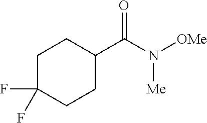 [SMILES: CON(C)C(=O)C1CCC(F)(F)CC1] | 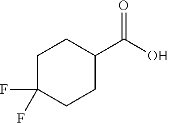 [SMILES: O=C(O)C1CCC(F)(F)CC1] | Modification: The reaction was run with 4,4- difluorocyanohexanecarboxylic acid, 1.05 equiv. of N,O-dimethylhydroxylamine hydrochloride, and 1.2 equiv. each of CDI nd DIPEA. The crude was purified by silica gel column (PE/EtOAc = 3:1, (PMA, 300° C., 10 s, R_{f} = 0.6)) to afford 4,4-difluoro- N-methoxy-N-methyl-cyclohexanecarboxamide (Int-D39, 4.00 g, 19.3 mmol, 79% yield) as an oil. ¹H |
| | | NMR (400 MHz, CDCl₃) δ 1.69-1.90 (m, 6H) 2.12- |
| Int-D39 | | 2.26 (m, 2H) 2.66-2.81 (m, 1H) 3.19 (s, 3H) 3.72 |
| | | (s, 3H). |

### Method Int 38. Intermediate Int-A43: (3-chloro-5,6-dimethylpyrazin-2-yl)(3-(2-fluoroethyl) bicyclo[1.1.1]pentan-1-yl) methanone

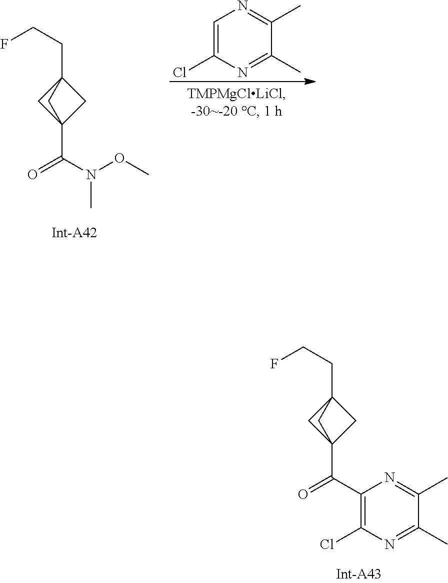

A mixture of 3-(2-fluoroethyl)-N-methoxy-N-methyl-bicyclo[1.1.1]pentane-1-carboxamide (Int-A42, 1 equiv., 120 mg, 0.596 mmol) and 5-chloro-2,3-dimethyl-pyrazine (1.5 equiv., 0.11 mL, 0.894 mmol) in THF (3 mL) was degassed with N₂ (×3). The mixture was cooled to-20° C., TMPMgCl·LiCl (1.3 equiv., 0.78 mL, 0.775 mmol) was added dropwise at −20° C., and the mixture was stirred at −20° C. for 1 h. LCMS showed most of starting material remained. TMPMgCl·LiCl (1.2 equiv., 0.72 mL, 0.716 mmol) was added at −20° C. and stirred at −20° C. for 1 h. LCMS showed 29% of desired product. TLC showed the starting material was consumed completed and one new spot found. The reaction mixture was added to NH₄Cl (aq, 10 mL), partitioned between EtOAc (2×40 mL) and water (40 mL) and extracted. The combined organic layers were dried over Na₂SO₄, filtered, and concentrated under reduced pressure to give a residue. The crude product was purified by silica gel column chromatography (0-30% EtOAc in PE)(TLC, 25% EtOAc in PE, desired product R_{f}=0.6) to give (3-chloro-5,6-dimethylpyrazin-2-yl)(3-(2-fluoroethyl) bicyclo[1.1.1]pentan-1-yl) methanone (Int-A43, 90 mg, 0.318 mmol, 53% yield) as a solid. LCMS: [M+H]⁺=285.0; purity=86% (220 nm); Ret. time=0.573 min. ¹H NMR (400 MHz, CDCl₃) δ 4.59-4.43 (m, 2H), 2.57 (d, J=5.9 Hz, 6H), 2.20-2.16 (m, 6H), 1.97-1.86 (m, 2H).

An analogous method was followed to obtain the following intermediates.

| Structure | Starting Material | Characterization |
|---|---|---|
| 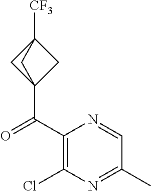 [SMILES: Cc1cnc(C(=O)C23CC(C(F)(F)F)(C2)C3)c(Cl)n1] | 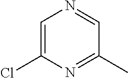 | Modification: The reaction was run with N- methoxy-N-methyl-3- (trifluoromethyl)bicyclo[1.1.1]pentane-1- carboxamide in anhydrous DME at −15° C. for 2 h. The crude mixture was purified by silica gel flash- column chromatography using an elution gradient of 2%-10% EtOAc in hexanes to yield (3-chloro- 5-methylpyrazin-2-yl)(3- (trifluoromethyl)bicyclo[1.1.1]pentan-1- yl)methanone (Int-C20, 40% yield) as a solid. ESI- MS: [M + H]⁺ = 289.0; [M + Na]⁺ = 316.1. ¹H NMR (CDCl₃, 400 MHz): δ 8.41 (1H, s), 2.64 (3H, s), |
| | | 2.44 (6H, s). ¹⁹F NMR (CDCl₃, 376 MHz): δ −73.4 |
| Int-C20 | 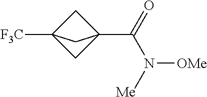 [SMILES: CON(C)C(=O)C12CC(C(F)(F)F)(C1)C2] | (3F, s). |
| 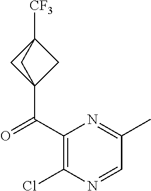 [SMILES: Cc1cnc(Cl)c(C(=O)C23CC(C(F)(F)F)(C2)C3)n1] | 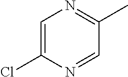 | Modification: The reaction was run with N- methoxy-N-methyl-3- (trifluoromethyl)bicyclo[1.1.1]pentane-1- carboxamide in anhydrous DME at −15° C. for 2 h. The crude mixture was purified by silica gel flash- column chromatography using an elution gradient of 2%-10% EtOAc in hexanes to yield (3-chloro- 6-methyl-pyrazin-2-yl)-[3-(trifluoromethyl)-1- bicyclo[1.1.1]pentanyl]methanone (Int-C30, 2.57 g, 7.79 mmol, 78% yield) as a crystalline solid. ESI-MS: [M + H]⁺ = 291.2. ¹H NMR: (CDCl₃, 400 MHz): δ 8.37 (1H, s), 2.61 (3H, s), 2.44 (6H, s). |
| Int-C30 | 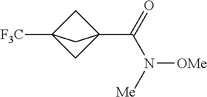 [SMILES: CON(C)C(=O)C12CC(C(F)(F)F)(C1)C2] | |
| 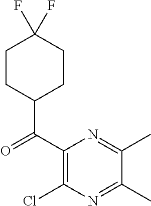 [SMILES: Cc1nc(Cl)c(C(=O)C2CCC(F)(F)CC2)nc1C] | 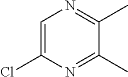 | Modification: The reaction was run in with Int- D39 and 1.5 equiv. each of 5-chloro-2,3-dimethyl- pyrazine, and TMPMgCl•LiCl. The crude was then purified by reversed-phase HPLC (FA condition) to give (3-chloro-5,6-dimethyl-pyrazin-2-yl)-(4,4- difluorocyclohexyl)methanone (Int-D40, 4000 mg, 13.9 mmol, 72% yield) as a solid. LCMS: [M + H]⁺ = 289.0. ¹H NMR (400 MHz, CDCl₃) δ 1.80-2.02 (m, 6H) 2.12-2.25 (m, 2H) 2.59 (d, J = 7.3 Hz, 6H) 3.63 (br dd, J = 5.9, 3.4 Hz, 1H). |
| Int-D40 | 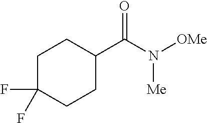 [SMILES: CON(C)C(=O)C1CCC(F)(F)CC1] | |
| | Int-D39 | |
| 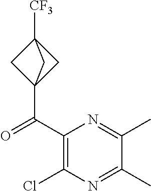 [SMILES: Cc1nc(Cl)c(C(=O)C23CC(C(F)(F)F)(C2)C3)nc1C] | 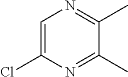 | Modifications: The reaction was run with 2,3- dimethyl-5-chloropyrazine and N-methoxy-N- methyl-3-(trifluoromethyl)bicyclo[1.1.1]pentane- 1-carboxamide, at −45° C. (dry ice/acetonitrile bath) for 1 h. The crude residue was purified by flash chromatography (220 g SiO₂ column, 2-15% EtOAc in hexanes elution gradient) to afford (3- chloro-5,6-dimethyl-pyrazin-2-yl)-[3- (trifluoromethyl)-1- bicyclo[1.1.1]pentanyl]methanone (Int-F1, 9.6 g, 18.3 mmol, 94% yield) as a solid. ¹H NMR (CDCl₃, 400 MHz): δ 2.59 (3H, s), 2.58 (3H, s), |
| | | 2.43 (6H, s). ¹⁹F NMR (CDCl₃, 376 MHz): δ −73 3 |
| Int-F1 | 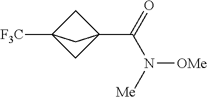 [SMILES: CON(C)C(=O)C12CC(C(F)(F)F)(C1)C2] | (3F, s). |

### Method Int 39. Intermediate Int-A44:1-cyclopropyl-6-oxo-1,6-dihydropyridine-3-carbaldehyde

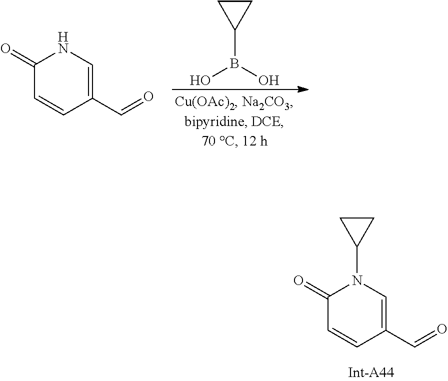 [SMILES: O=Cc1ccc(=O)[nH]c1 ; OB(O)C1CC1 ; O=Cc1ccc(=O)n(C2CC2)c1]

A mixture of 6-oxo-1H-pyridine-3-carbaldehyde (1 equiv., 2.0 g, 16.2 mmol) in DCE (200 mL) was added cyclopropylboronic acid (1.5 equiv., 2.09 g, 24.4 mmol), Cu(OAc)₂ (1.5 equiv., 4.43 g, 24.4 mmol), Na₂CO₃ (3 equiv., 5.17 g, 48.7 mmol) and bipyridine (1 equiv., 2.54 g, 16.2 mmol), then stirred at 70° C. for 12 h under O₂ atmosphere. LCMS showed 62% of desired product. The reaction mixture was adjusted to pH=7 by 1 M HCl (200 mL) and partitioned between DCM (2×150 mL). The combined organic layers were dried over Na₂SO₄, filtered, and concentrated under reduced pressure to give a residue. The crude residue was purified by silica gel column chromatography (0-100% petroleum ether in EtOAc)(TLC, 50% petroleum ether in EtOAc, desired product R_{f}=0.3) to give 1-cyclopropyl-6-oxo-1,6-dihydropyridine-3-carbaldehyde (Int-A44, 1.37 g, 8.4 mmol, 52% yield) as a solid. LCMS: [M+H]⁺=164.1; purity=95% (220 nm); Ret. time=0.315 min. ¹H NMR (400 MHz, CDCl₃) δ 9.61 (s, 1H), 7.93 (d, J=2.1 Hz, 1H), 7.78 (dd, J=2.3, 9.4 Hz, 1H), 6.58 (d, J=9.5 Hz, 1H), 3.48-3.36 (m, 1H), 1.30-1.16 (m, 2H), 1.00-0.88 (m, 2H).

### Method Int 40. Intermediate Int-A45:5-(4-bromotetrahydro-2H-pyran-2-yl)-1-cyclopropylpyridin-2 (1H)-one

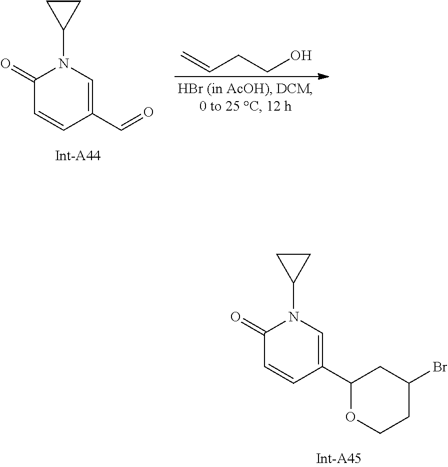

A mixture of 1-cyclopropyl-6-oxo-pyridine-3-carbaldehyde (Int-A44, 1 equiv., 1.5 g, 9.19 mmol) and 3-Buten-1-ol (1 equiv., 0.79 mL, 9.19 mmol) in DCM (20 mL) was added dropwise HBr in AcOH (3 equiv., 4.9 mL, 27.6 mmol) at 0° C. under N₂ atmosphere, then stirred at 25° C. for 12 h under N₂ atmosphere. LCMS showed 68% of desired product. The reaction mixture was adjusted to pH=7 by NaHCO₃ (200 mL) and partitioned between EtOAc (2×200 mL). The combined organic layers were dried over Na₂SO₄, filtered, and concentrated under reduced pressure to give a residue. The crude residue was purified by silica gel column chromatography (0-100% petroleum ether in EtOAc)(TLC, 100% EtOAc, desired product R_{f}=0.2) to give 5-(4-bromotetrahydro-2H-pyran-2-yl)-1-cyclopropylpyridin-2 (1H)-one (Int-A45, 2.0 g, 6.71 mmol, 73% yield) as an oil. LCMS: [M+H]⁺=300.1; purity=89% (220 nm); Ret. time=0.457 min. ¹H NMR (400 MHz, CDCl₃) δ 7.33-7.27 (m, 2H), 6.55 (d, J=9.6 Hz, 1H), 4.82-4.59 (m, 0.5H), 4.20-4.03 (m, 2H), 3.60-3.49 (m, 0.5H), 3.36-3.25 (m, 1H), 2.17 (br dd, J=4.8, 6.9 Hz, 2H), 2.05 (s, 3H), 1.14 (br d, J=6.6 Hz, 2H), 0.91-0.84 (m, 2H).

### Method Int 41. Intermediate Int-A46:1-isopropyl-6-oxo-pyridine-3-carbaldehyde

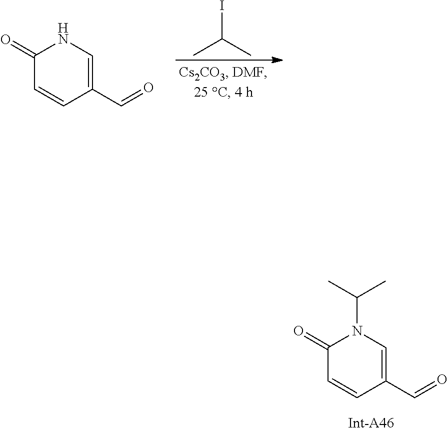

To a solution of 6-oxo-1H-pyridine-3-carbaldehyde (1 equiv., 8.0 g, 65.0 mmol) and Cs₂CO₃ (2 equiv., 42.3 g, 130 mmol) in DMF (300 mL) was added 2-iodopropane (1.1 equiv., 7.1 mL, 71.5 mmol) slowly. The mixture was stirred at 25° C. for 4 hours. TLC showed raw material was consumed completely, and two spots were formed. The reaction mixture was filtered and the filter cake was washed with EtOAc (2×50 mL). The filtrate was concentrated and extracted with EtOAc (3×500 mL), and the combined organic layers were dried over Na₂SO₄, filtered, and concentrated under reduced pressure to give a residue. The residue was purified by silica gel column chromatography (0-100% EtOAc in PE) (TLC, 100% EtOAc, desired product R_{f}=0.40) to afford the product 1-isopropyl-6-oxo-pyridine-3-carbaldehyde (Int-A46, 3.6 g, 20.5 mmol, 32% yield) as a solid. ¹H NMR (400 MHz, CDCl₃) δ 9.65 (d, J=0.6 Hz, 1H), 7.96 (d, J=2.4 Hz, 1H), 7.78 (dd, J=2.4, 9.4 Hz, 1H), 6.61 (d, J=9.4 Hz, 1H), 5.26 (td, J=6.8, 13.7 Hz, 1H), 1.44 (d, J=6.9 Hz, 6H).

An analogous method was followed to obtain the following intermediates.

| | Starting | |
|---|---|---|
| Structure | Material | Characterization |
| 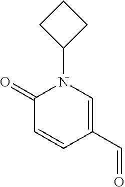 [SMILES: O=Cc1ccc(=O)n(C2CCC2)c1] | 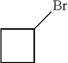 | Modifications: The reaction was run with two equiv. each of bromocyclobutane and Cs2CO3 at 90° C. for 12 h. The crude residue was purified by flash chromatography (330 g SiO₂ column, 0-58% ethyl acetate in petroleum ether gradient) to afford 1- cyclobutyl-6-oxo-pyridine-3-carbaldehyde (5.70 g, 32.2 mmol, 40% yield) as a solid. LCMS: [M + H]⁺ = 178.2. ¹H NMR (400 MHz, CDCl₃) δ 1.88-1.98 (m, 2 H) 2.26 (qd, J = 9.68, 2.69 Hz, 2 H) 2.53-2.62 (m, 2 H) 5.02-5.12 (m, 1 H) 6.57 (d, J = 9.38 Hz, 1 H) 7.79 (dd, J = 9.44, 2.31 Hz, 1 H) 8.03 (d, J = 2.25 Hz, 1 H) |
| | | 9.66 (s, 1 H). |
| Int-F13 | | |

### Method Int 42. Intermediate Int-A47:5-(4-bromotetrahydropyran-2-yl)-1-isopropyl-pyridin-2-one

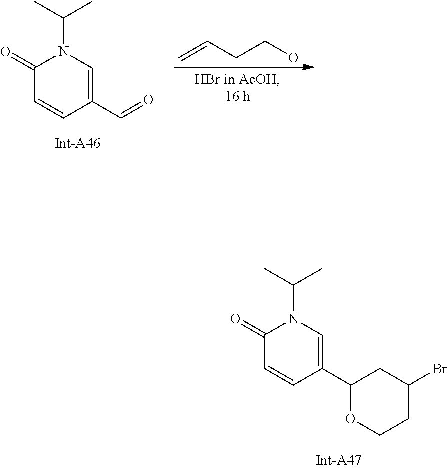

To a solution of 1-isopropyl-6-oxo-pyridine-3-carbaldehyde (Int-A46, 1 equiv., 3.5 g, 21.2 mmol) and 3-buten-1-ol (5 equiv., 9.1 mL, 106 mmol) in DCM (120 mL) was added HBr in AcOH (5 equiv., 6.3 mL, 106 mmol) dropwise at 0° C. under nitrogen atmosphere, and the mixture was stirred at 25° C. for 16 hours. LCMS showed desired product (44%). The reaction mixture was adjusted to pH=7 with NaHCO₃ aqueous solution. The reaction mixture was extracted with DCM (3×200 mL), the combined organic layers were dried over Na₂SO₄, filtered, and concentrated under reduced pressure to give a residue. The reaction was purified by reversed-phase chromatography (65-90% [water (FA)-MeCN]) and concentrated to give the 5-(4-bromotetrahydropyran-2-yl)-1-isopropyl-pyridin-2-one (Int-A47, 2.8 g, 8.4 mmol, 40% yield) as an oil. LCMS: [M+H]⁺=302.1; purity=90% (220 nm); Ret. time=0.596 min. 1H NMR (400 MHz, CDCl₃) δ7.36-7.31 (m, 1H), 7.31-7.27 (m, 1H), 6.60 (d, J=9.3 Hz, 1H), 5.27 (spt, J=6.8 Hz, 1H), 4.35-4.04 (m, 3H), 3.65-3.46 (m, 1H), 2.43 (td, J=2.1, 12.9 Hz, 1H), 2.30-1.93 (m, 4H), 1.36 (dd, J=2.6, 6.9 Hz, 6H).

An analogous method was followed to obtain the following intermediates.

| | Starting | |
|---|---|---|
| Structure | Material | Characterization |
| 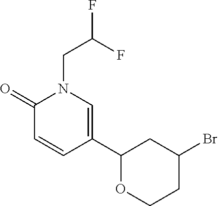 [SMILES: O=c1ccc(C2CC(Br)CCO2)cn1CC(F)F] | 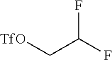 [SMILES: O=S(=O)(OCC(F)F)C(F)(F)F] | Modification: The first reaction was ran using the intermediate to the left, and ran in ACN. The residue was purified by silica gel column chromatography (0-50% EtOAc in Petroleum ether) (TLC, 50% EtOAc in PE, desired product R_{f} = 0.4) to give 1.3 g crude product, the crude product was purified by Prep-HPLC (Condition 8, Gradient a) and lyophilized to afford the 5-(4-bromotetrahydropyran- 2-y1)-1-(2,2-difluoroethyl)pyridin-2-one (Int-A48, 1.0 g, 2.79 mmol, 62% yield) as an oil. LCMS: [M + H]⁺ = 323.9; purity = 97% (220 nm); Ret. time = 0.475 min. ¹H NMR (400 MHz, CDCl₃) δ 7.38 (dd, J = 2.3, 9.5 Hz, 1H), 7.28 (s, 1H), 6.64 (d, J = 9.4 Hz, 1H), |
| | | 6.30-5.93 (m, 1H), 4.24 (br d, J = 4.4 Hz, 3H), 4.15- |
| Int-A48 | | 4.05 (m, 2H), 3.55 (dt, J = 1.8, 12.1 Hz, 1H), 2.43 (td, |
| | | J = 1.9, 12.9 Hz, 1H), 2.29-2.21 (m, 1H), 2.18-2.07 |
| | | (m, 1H), 2.05-1.91 (m, 1H). |
| 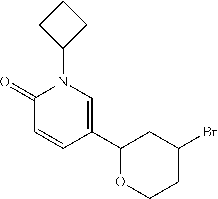 [SMILES: O=c1ccc(C2CC(Br)CCO2)cn1C1CCC1] | 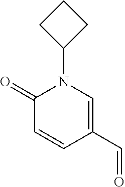 [SMILES: O=Cc1ccc(=O)n(C2CCC2)c1] | Modifications: The reaction was run with 1.2 equiv. 3-buten-1-ol and 3 equiv. HBr (in AcOH) for 12 h. The crude residue was purified by flash chromatography (120 g SiO₂ column, 0-65% EtOAc in PE) and then purified by prep-HPLC (Condition 19, Gradient a) to afford 5-(4-bromotetrahydropyran-2- yl)-1-cyclobutyl-pyridin-2-one (Int-F14, 5.70 g, 18.3 mmol, 69% yield) as a gum. LCMS: [M + H]⁺ = 313.9. ¹H NMR (400 MHz, CDCl₃) δ 1.81-1.91 (m, 2 H) 1.93-2.32 (m, 7 H) 2.39-2.55 (m, 3 H) 3.56 (td, J = 12.13, 2.13 Hz, 1 H) 4.00 (br dd, J = 11.94, 4.82 Hz, 1 H) 4.08-4.17 (m, 2 H) 4.23 (tt, J = 11.87, 4.46 Hz, 1 |
| | | H) 4.65-4.84 (m, 1 H) 5.06-5.18 (m, 1 H) 6.53 (d, |
| Int-F14 | Int-F13 | J = 9.38 Hz, 1 H) 7.27-7.31 (m, 1 H) 7.41-7.46 (m, 1 |
| | | H). |

### Method Int 43. Intermediate Int-A23: (2R,4S)—N-[3-[3-(1,1-difluoroethyl) bicyclo[1.1.1]pentane-1-carbonyl]-5,6-dimethyl-pyrazin-2-yl]-2-(1-methyl-6-oxo-3-pyridyl)tetrahydropyran-4-carboxamide

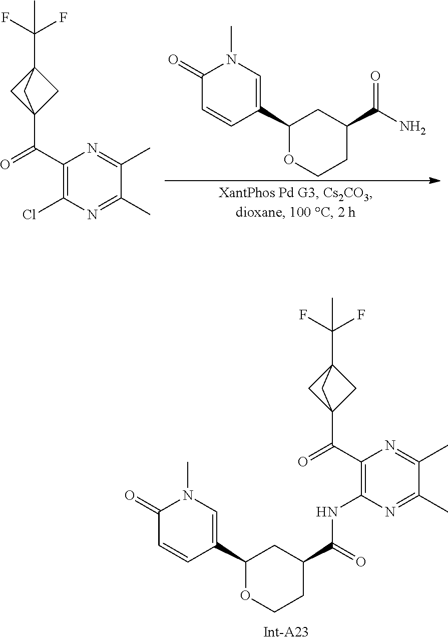 [SMILES: Cc1nc(Cl)c(C(=O)C23CC(C(C)(F)F)(C2)C3)nc1C ; Cn1cc([C@H]2C[C@@H](C(N)=O)CCO2)ccc1=O ; Cc1nc(NC(=O)[C@H]2CCO[C@@H](c3ccc(=O)n(C)c3)C2)c(C(=O)C23CC(C(C)(F)F)(C2)C3)nc1C]

To a glass vial equipped with a teflon-coated magnetic stirring bar was added (3-chloro-5,6-dimethyl-pyrazin-2-yl)-[3-(1,1-difluoroethyl)-1-bicyclo[1.1.1]pentanyl]methanone (1.2 equiv., 305 mg, 1.02 mmol), (2R,4S)-2-(1-methyl-6-oxo-3-pyridyl)tetrahydropyran-4-carboxamide (1 equiv., 200 mg, 0.846 mmol), Cs₂CO₃ (1 equiv., 276 mg, 0.846 mmol) and anhydrous 1,4-dioxane (10 mL). The vial was capped and degassed with Ar for 5 min. XantPhos Pd G₃ (0.1 equiv., 80 mg, 0.085 mmol) was added, the reaction was stirred at 100° C. for 2 hours under N₂. LCMS showed starting material was consumed completely and desired mass was detected. The reaction mixture was filtered and concentrated under reduced pressure to give a residue. The residue was purified by silica gel column chromatography (25% EtOAc in PE, then 17% MeOH in EtOAc)(TLC, 5% MeOH in DCM, R_{f}=0.4) to give (2R,4S)—N-[3-[3-(1,1-difluoroethyl) bicyclo[1.1.1]pentane-1-carbonyl]-5,6-dimethyl-pyrazin-2-yl]-2-(1-methyl-6-oxo-3-pyridyl)tetrahydropyran-4-carboxamide (Int-A23, 350 mg, 0.699 mmol, 83% yield) as an oil. LCMS: [M+H]⁺=501.2; purity=98% (220 nm); Ret. time=0.520 min. ¹HNMR (400 MHz, CDCl₃) δ 1.61 (t, J=18.2 Hz, 3H) 1.76-1.86 (m, 1H) 1.93-2.03 (m, 2H) 2.11-2.19 (m, 1H) 2.40 (s, 6H) 2.54-2.64 (m, 6H) 2.86-2.99 (m, 1H) 3.55 (s, 3H) 3.60-3.71 (m, 1H) 4.14-4.29 (m, 2H) 6.58 (d, J=10.1 Hz, 1H) 7.32-7.38 (m, 2H) 11.17 (s, 1H).

### Method Int 44. Intermediate Int-A49: (1-methylcyclopropyl) methyl methanesulfonate

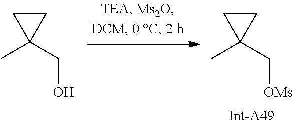

To a solution of (1-methylcyclopropyl) methanol (1 equiv., 0.14 mL, 2.32 mmol) and TEA (3 equiv., 0.97 mL, 6.97 mmol) in DCM (6 mL) was added methanesulfonic anhydride (1.5 equiv., 607 mg, 3.48 mmol) at 0° C. and then the mixture was stirred for 2 h at 0° C. The reaction solution was extracted with EtOAc (2×10 mL), then the organics were washed with 10 mL saturated brine solution. The combined organic layers were dried over Na₂SO₄, filtered, and concentrated under reduced pressure to give (1-methylcyclopropyl) methyl methanesulfonate (Int-A49, 150 mg, 0.913 mmol, 39% yield) as a liquid, which was used directly.

### Method Int 45. Intermediate Int-B1:5-[4-[4-[2-fluoro-4-(trifluoromethyl)phenyl]-7-methyl-pteridin-2-yl]tetrahydropyran-2-yl]-1-methyl-pyridin-2-one

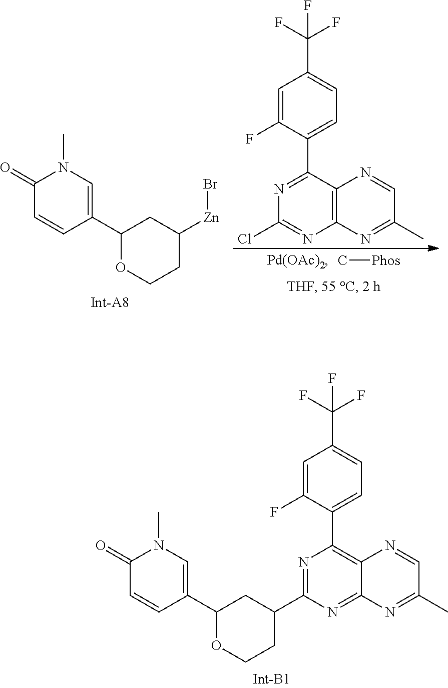

To a solution of 2-chloro-4-[2-fluoro-4-(trifluoromethyl)phenyl]-7-methyl-pteridine (1 equiv., 800 mg, 2.33 mmol), Pd(OAc)₂ (0.1 equiv., 53 mg, 0.233 mmol) and C-Phos (0.2 equiv., 204 mg, 0.467 mmol) in THF (3 mL) was purged with N₂ for 3 times, then bromo-[2-(1-methyl-6-oxo-3-pyridyl)tetrahydropyran-4-yl]zinc (Int-A8, 1.2 equiv., 946 mg, 2.80 mmol) was added, the reaction solution was stirred at 55° C. for 2 hours. LCMS showed ~40% of desired product was detected. The mixture was quenched by H₂O (200 mL), extracted with DCM (3× 150 mL), the combined organic layers was dried over anhydrous Na₂SO₄, filtered, and concentrated under reduced pressure to give the residue. The residue was purified by silica gel column chromatography (0-100% EtOAc in PE, then 0-50% MeOH in EtOAc)(TLC, 100% EtOAc, desired product R_{f}-0.1) to give the 5-[4-[4-[2-fluoro-4-(trifluoromethyl)phenyl]-7-methyl-pteridin-2-yl]tetrahydropyran-2-yl]-1-methyl-pyridin-2-one (Int-B1, 500 mg, 1 mmol, 43% yield) as a solid. LCMS: [M+H]⁺=500.1; purity=92% (UV 220 nm); Ret. time =0.526 min. ¹H NMR (400 MHz, CDCl₃) δ 8.88-8.83 (m, 1H), 7.92-7.81 (m, 1H), 7.65 (br d, J=7.9 Hz, 1H), 7.59-7.53 (m, 1H), 7.43-7.39 (m, 2H), 7.36-7.29 (m, 1H), 6.63-6.54 (m, 1H), 4.39-4.28 (m, 2H), 3.82 (dt, J=2.8, 11.7 Hz, 1H), 3.65-3.59 (m, 1H), 3.58-3.52 (m, 4H), 2.96-2.90 (m, 3H), 2.28-2.12 (m, 3H).

### Method Int 46. Intermediate Int-B2: methyl-3-[[(2R,4S)-2-(6-benzyloxy-3-pyridyl)tetrahydropyran-4-carbonyl]amino]-6-methoxy-5-methyl-pyrazine-2-carboxylate

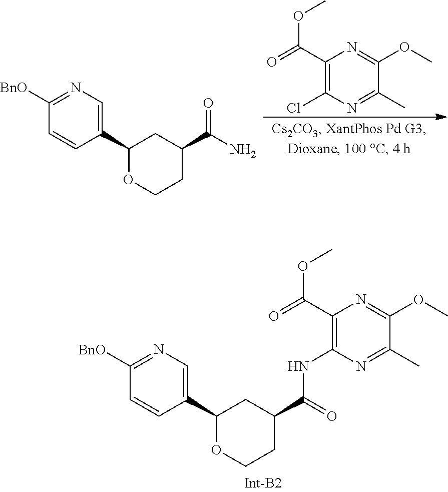 [SMILES: NC(=O)[C@H]1CCO[C@@H](c2ccc(OCc3ccccc3)nc2)C1 ; COC(=O)c1nc(OC)c(C)nc1Cl ; COC(=O)c1nc(OC)c(C)nc1NC(=O)[C@H]1CCO[C@@H](c2ccc(OCc3ccccc3)nc2)C1]

To a mixture of (2R,4S)-2-(6-benzyloxy-3-pyridyl)tetrahydropyran-4-carboxamide (1 equiv., 2.8 g, 8.96 mmol) in 1,4-dioxane (45 mL) was added methyl 3-chloro-6-methoxy-5-methyl-pyrazine-2-carboxylate (1.2 equiv., 2.33 g, 10.8 mmol), Cs₂CO₃ (2 equiv., 5.84 g, 17.9 mmol) and XantPhos Pd G3 (0.1 equiv., 0.85 g, 0.896 mmol) at 20° C. The mixture was degassed with N₂ for 3 times. The mixture was stirred at 105° C. for 4 h. LCMS showed starting material was consumed completely and desired mass was detected. The solution (combined with another batch) was extracted with EtOAc (3×50 mL), and the organics were washed with 100 mL saturated brine solution. The combined organic layers were dried over anhydrous Na₂SO₄, filtered, and concentrated under reduced pressure to give a crude residue. The crude was purified by silica gel column chromatography (67% EtOAc in PE, R_{f}=0.5) to give methyl-3-[[(2R,4S)-2-(6-benzyloxy-3-pyridyl)tetrahydropyran-4-carbonyl]amino]-6-methoxy-5-methyl-pyrazine-2-carboxylate (Int-B2, 3.3 g, 5.76 mmol, 64% yield) as a solid. LCMS: [M+H]⁺=493.2; Purity =87% (220 nm); Ret. time=0.551 min. ¹H NMR (400 MHz, CDCl₃) δ 1.86-2.06 (m, 3H), 2.12-2.22 (m, 1H), 2.57 (s, 3H), 2.83-3.00 (m, 1H), 3.64-3.78 (m, 1H), 4.00 (d, J=9.4 Hz, 6H), 4.23-4.31 (m, 1H), 4.40 (dd, J=11.4, 2.0 Hz, 1H), 5.39 (s, 2H), 6.82 (d, J=8.5 Hz, 1H), 7.29-7.41 (m, 3H), 7.42-7.50 (m, 2H), 7.66 (dd, J=8.57, 2.4 Hz, 1H), 8.16 (d, J=2.4 Hz, 1H), 10.35 (s, 1H).

### Method Int 47. Intermediate Int-B3:2-[(2R,4S)-2-(6-benzyloxy-3-pyridyl)tetrahydropyran-4-yl]-6-methoxy-7-methyl-3H-pteridin-4-one

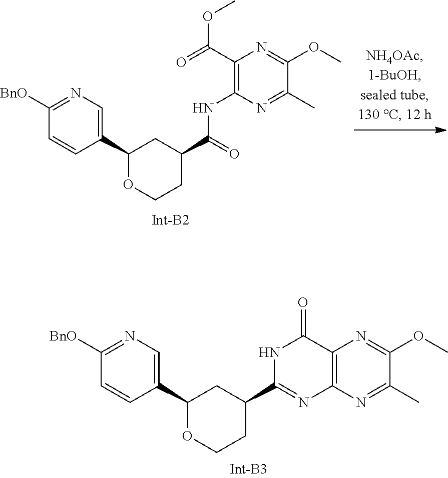 [SMILES: COC(=O)c1nc(OC)c(C)nc1NC(=O)[C@H]1CCO[C@@H](c2ccc(OCc3ccccc3)nc2)C1 ; COc1nc2c(=O)[nH]c([C@H]3CCO[C@@H](c4ccc(OCc5ccccc5)nc4)C3)nc2nc1C]

To a solution of methyl 3-[[(2R,4S)-2-(6-benzyloxy-3-pyridyl)tetrahydropyran-4-carbonyl]amino]-6-methoxy-5-methyl-pyrazine-2-carboxylate (Int-B2, 1 equiv., 2.35 g, 4.77 mmol) in 1-Butanol (25 mL) was added ammonium acetate (20 equiv., 7.36 g, 95.4 mmol). Then the reaction mixture was stirred at 130° C. for 12 h in sealed tube. LCMS showed starting material was consumed completely and desired mass was detected. The reaction mixture was filtered, concentrated under reduced pressure, and triturated with MTBE (50 mL) to give 2-[(2R,4S)-2-(6-benzyloxy-3-pyridyl)tetrahydropyran-4-yl]-6-methoxy-7-methyl-3H-pteridin-4-one (Int-B3, 2.2 g, 4.79 mmol, 100% yield) as a solid. LCMS: [M+H]⁺=460.2; Purity=96% (220 nm); Ret. time=0.513 min. ¹H NMR (400 MHz, CDCl₃) δ 11.07-10.71 (m, 1H), 8.18 (d, J=2.1 Hz, 1H), 7.69 (dd, J=2.4, 8.6 Hz, 1H), 7.49-7.42 (m, 2H), 7.41-7.30 (m, 3H), 6.80 (d, J=8.6 Hz, 1H), 5.38 (s, 2H), 4.56-4.47 (m, 1H), 4.33 (br dd, J=3.2, 11.7 Hz, 1H), 4.18 (s, 3H), 3.86-3.73 (m, 1H), 3.26-3.10 (m, 1H), 2.68 (s, 3H), 2.33-2.01 (m, 4H).

An analogous procedure was followed to obtain the following intermediates.

| Structure | Starting Material | Characterization |
|---|---|---|
| 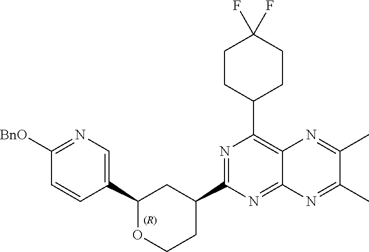 | 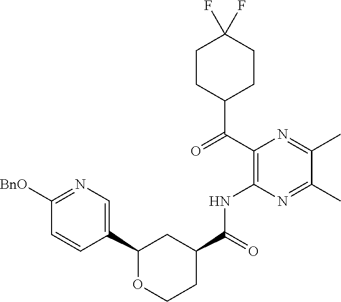 | Modification: The reaction was run at 115° C. for 1 h to afford 2-[(2R,4S)- 2-(6-benzyloxy-3- pyridyl)tetrahydropyran-4-yl]-4-(4,4- difluorocyclohexyl)-6,7-dimethyl- pteridine (Int-D42, 2800 mg, 4.93 mmol, 93% yield) as a solid. LCMS: [M + H]⁺ = 546.3. |
| Int-D42 | Int-D41 | |
| 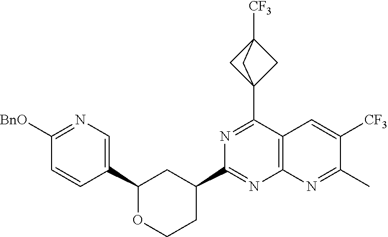 [SMILES: Cc1nc2nc([C@H]3CCO[C@@H](c4ccc(OCc5ccccc5)nc4)C3)nc(C34CC(C(F)(F)F)(C3)C4)c2cc1C(F)(F)F] | 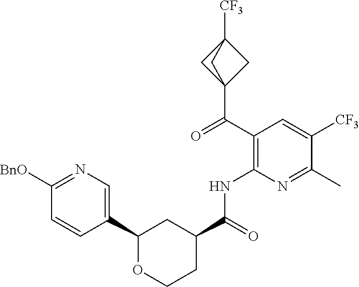 [SMILES: Cc1nc(NC(=O)[C@H]2CCO[C@@H](c3ccc(OCc4ccccc4)nc3)C2)c(C(=O)C23CC(C(F)(F)F)(C2)C3)cc1C(F)(F)F] | Modification: The reaction was run at 115° C. for 1 h to afford 2-[(2R,4S)- 2-(6-benzyloxy-3- pyridyl)tetrahydropyran-4-y1]-7- methyl-6-(trifluoromethyl)-4-[3- (trifluoromethyl)-1- bicyclo[1.1.1]pentanyl]pyrido[2,3- d]pyrimidine (Int-E9, 30 mg, 0.05 mmol, 99% yield) as a solid. |
| Int-E9 | Int-E8 | |
| 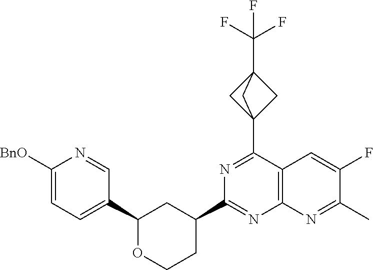 [SMILES: Cc1nc2nc([C@H]3CCO[C@@H](c4ccc(OCc5ccccc5)nc4)C3)nc(C34CC(C(F)(F)F)(C3)C4)c2cc1F] | 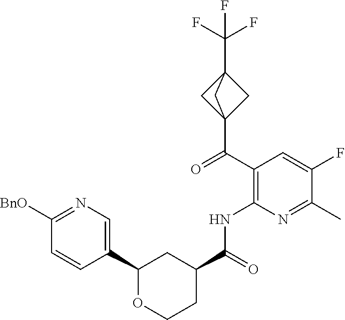 [SMILES: Cc1nc(NC(=O)[C@H]2CCO[C@@H](c3ccc(OCc4ccccc4)nc3)C2)c(C(=O)C23CC(C(F)(F)F)(C2)C3)cc1F] | Modification: The reaction was run at 115° C. for 1 h to afford 2-((2R,4S)- 2-(6-(benzyloxy)pyridin-3- yl)tetrahydro-2H-pyran-4-yl)-6- fluoro-7-methyl-4-(3- (trifluoromethyl)bicyclo[1.1.1] pentan-1-yl)pyrido[2,3-d]pyrimidine (Int-E23, 196 mg, 0.35 mmol, 96% yield) as a foam. |
| Int-E23 | Int-E22 | |
| 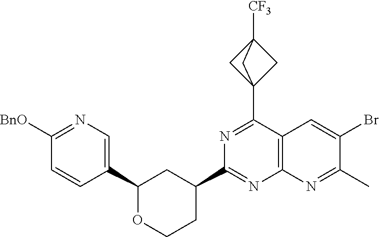 [SMILES: Cc1nc2nc([C@H]3CCO[C@@H](c4ccc(OCc5ccccc5)nc4)C3)nc(C34CC(C(F)(F)F)(C3)C4)c2cc1Br] | 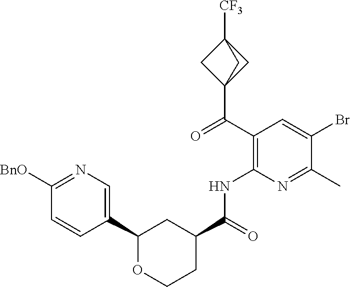 [SMILES: Cc1nc(NC(=O)[C@H]2CCO[C@@H](c3ccc(OCc4ccccc4)nc3)C2)c(C(=O)C23CC(C(F)(F)F)(C2)C3)cc1Br] | The procedure yielded 2-((2R,4S)-2- (6-(benzyloxy)pyridin-3- yl)tetrahydro-2H-pyran-4-yl)-6- bromo-7-methyl-4-(3- (trifluoromethyl)bicyclo[1.1.1] pentan-1-yl)pyrido[2,3-d] pyrimidine (Int-E30). |
| Int-E30 | Int-E29 | |
| 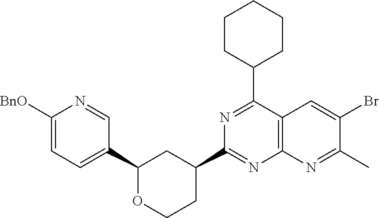 [SMILES: Cc1nc2nc([C@H]3CCO[C@@H](c4ccc(OCc5ccccc5)nc4)C3)nc(C3CCCCC3)c2cc1Br] | 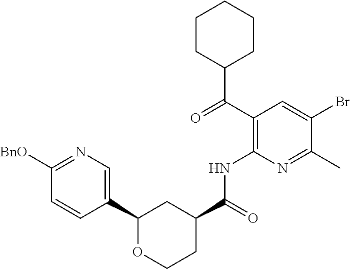 [SMILES: Cc1nc(NC(=O)[C@H]2CCO[C@@H](c3ccc(OCc4ccccc4)nc3)C2)c(C(=O)C2CCCCC2)cc1Br] | The crude mixture was purified by flash column chromatography (80 g, SiO₂ column, 20-60% EtOAc in hexanes elution gradient) to afford 2- [(2R,4S)-2-(6-benzyloxy-3- pyridyl)tetrahydropyran-4-y1]-6- bromo-4-cyclohexyl-7-methyl- pyrido[2,3-d]pyrimidine (Int-E39, 780 mg, 1.36 mmol, 63% yield) as a foam. |
| Int-E39 | Int-E38 | |
| 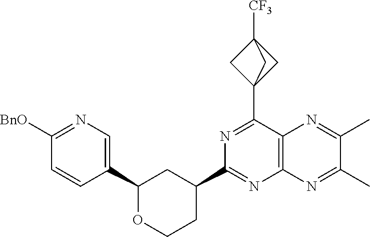 [SMILES: Cc1nc2nc([C@H]3CCO[C@@H](c4ccc(OCc5ccccc5)nc4)C3)nc(C34CC(C(F)(F)F)(C3)C4)c2nc1C] | 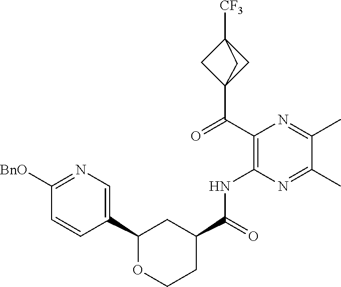 [SMILES: Cc1nc(NC(=O)[C@H]2CCO[C@@H](c3ccc(OCc4ccccc4)nc3)C2)c(C(=O)C23CC(C(F)(F)F)(C2)C3)nc1C] | The crude was purified by normal phase chromatography (0-80% EtOAc in hexanes) to afford 2- ((2R,4S)-2-(6-(benzyloxy)pyridin-3- yl)tetrahydro-2H-pyran-4-yl)-6,7- dimethyl-4-(3- (trifluoromethyl)bicyclo[1.1.1]pentan- 1-yl)pteridine (Int-F9, 2.4 g, 99% yield) as a solid. ¹H NMR (CDCl₃, 400 MHz): δ 8.17 (1H, d, J = 2.4 Hz), 7.68 (1H, dd, J = 8.6, 2.4 Hz), 7.41- 7.44 (2H, m), 7.33-7.37 (2H, m), 6.79 (1H, d, J = 8.6 Hz), 4.52 (1H, dd, J = 11.4, 2.1 Hz), 4.28-4.32 (1H, m), 3.78-3.85 (1H, m), 3.45-3.50 (1H, m), 2.79 (3H, s), 2.75 (3H, s), 2.63 (6H, s), 2.30 (1H, dd, J = 13.4, 3.0 |
| | | Hz), 2.12-2.16 (2H, m), 2.06-2.09 |
| Int-F9 | Int-F8 | (1H, m). ESI-MS: [M + H]⁺ = 562.4. |

### Method Int 48. Intermediate Int-B4:2-[(2R,4S)-2-(6-benzyloxy-3-pyridyl)tetrahydropyran-4-yl]-4-[2-fluoro-4-(trifluoromethyl)phenyl]-6-methoxy-7-methyl-pteridine

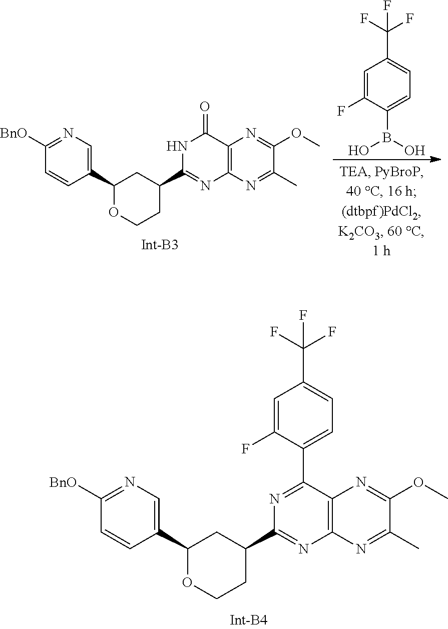 [SMILES: COc1nc2c(=O)[nH]c([C@H]3CCO[C@@H](c4ccc(OCc5ccccc5)nc4)C3)nc2nc1C ; OB(O)c1ccc(C(F)(F)F)cc1F ; COc1nc2c(-c3ccc(C(F)(F)F)cc3F)nc([C@H]3CCO[C@@H](c4ccc(OCc5ccccc5)nc4)C3)nc2nc1C]

A solution of 2-[(2R,4S)-2-(6-benzyloxy-3-pyridyl)tetrahydropyran-4-yl]-6-methoxy-7-methyl-3H-pteridin-4-one (Int-B3, 1 equiv., 1.9 g, 4.13 mmol) and TEA (3 equiv., 1.7 mL, 12.4 mmol), PyBroP (2 equiv., 3.86 g, 8.3 mmol) in THF (40 mL) was stirred for 16 h at 40° C. LCMS showed starting material was consumed completely and desired mass was detected (82%). Then [2-fluoro-4-(trifluoromethyl)phenyl]boronic acid (1.35 equiv., 1.16 g, 5.58 mmol), (dtbpf) PdCl₂ (0.1 equiv., 269 mg, 0.413 mmol) and K₂CO₃ (3 equiv., 1.7 g, 12.4 mmol), water (4 mL) was added to the mixture and the mixture was stirred for 1 h at 60° C. under N₂. LCMS showed starting material was consumed completely and the desired mass was detected (69%). The reaction was filtered and concentrated under reduced pressure to give the residue. The crude product was purified with Prep-TLC (PE:EtOAc=1:1, R_{f}=0.4) to give 2-[(2R,4S)-2-(6-benzyloxy-3-pyridyl)tetrahydropyran-4-yl]-4-[2-fluoro-4-(trifluoromethyl)phenyl]-6-methoxy-7-methyl-pteridine (Int-B4, 1.9 g, 3.04 mmol, 74% yield) as a solid. LCMS: [M+H]⁺=606.2; Purity=97% (220 nm); Ret. time=0.691 min. ¹H NMR (400 MHz, CDCl₃) δ 8.19 (d, J=2.3 Hz, 1H), 7.87 (t, J=7.3 Hz, 1H), 7.71 (dd, J=2.3, 8.6 Hz, 1H), 7.62 (d, J=8.0 Hz, 1H), 7.52 (d, J=9.8 Hz, 1H), 7.48-7.42 (m, 2H), 7.37 (t, J=7.4 Hz, 2H), 7.34-7.29 (m, 1H), 6.81 (d, J=8.5 Hz, 1H), 5.39 (s, 2H), 4.61-4.50 (m, 1H), 4.34 (br dd, J=3.0, 10.3 Hz, 1H), 4.01 (s, 3H), 3.85 (dt, J=3.6, 11.2 Hz, 1H), 3.66-3.52 (m, 1H), 2.77 (s, 3H), 2.38 (br d, J=13.5 Hz, 1H), 2.30-2.09 (m, 3H).

### Method Int 49. Intermediate Int-B5:5-[(2R,4S)-4-[4-[2-fluoro-4-(trifluoromethyl)phenyl]-6-methoxy-7-methyl-pteridin-2-yl]tetrahydropyran-2-yl]-1H-pyridin-2-one

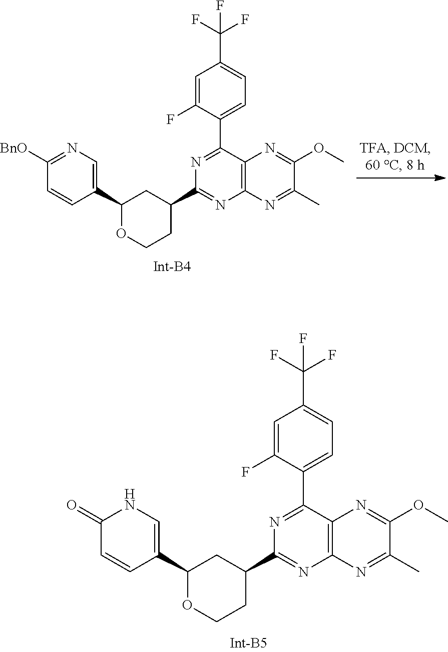 [SMILES: COc1nc2c(-c3ccc(C(F)(F)F)cc3F)nc([C@H]3CCO[C@@H](c4ccc(OCc5ccccc5)nc4)C3)nc2nc1C ; COc1nc2c(-c3ccc(C(F)(F)F)cc3F)nc([C@H]3CCO[C@@H](c4ccc(=O)[nH]c4)C3)nc2nc1C]

To a solution of 2-[(2R,4S)-2-(6-benzyloxy-3-pyridyl)tetrahydropyran-4-yl]-4-[2-fluoro-4-(trifluoromethyl)phenyl]-6-methoxy-7-methyl-pteridine (Int-B4, 1 equiv., 1.80 g, 2.97 mmol) in DCM (9 mL) was added TFA (9.0 mL) and then the mixture was stirred for 8 h at 60° C. LCMS showed the starting material was consumed completely and desired mass was detected (76%). The reaction solution was added to NaHCO₃(aq) dropwise to adjust pH >=7. Then the reaction solution was extracted with EtOAc (3×20 mL), and the organics was washed with 60 mL saturated brine solution. The combined organic layers were dried over anhydrous Na₂SO₄, filtered, and concentrated under reduced pressure to give a crude residue. The crude was used without purification and as batch 1:5-[(2R,4S)-4-[4-[2-fluoro-4-(trifluoromethyl)phenyl]-6-methoxy-7-methyl-pteridin-2-yl]tetrahydropyran-2-yl]-1H-pyridin-2-one (Int-B5 batch 1, 200 mg, 0.358 mmol, 12% yield) as a solid, and as batch 2:5-[(2R,4S)-4-[4-[2-fluoro-4-(trifluoromethyl)phenyl]-6-methoxy-7-methyl-pteridin-2-yl]tetrahydropyran-2-yl]-1H-pyridin-2-one (Int-B5 batch 2, 270 mg, 0.277 mmol, 9% yield) as a solid, and as batch 3:5-[(2R,4S)-4-[4-[2-fluoro-4-(trifluoromethyl)phenyl]-6-methoxy-7-methyl-pteridin-2-yl]tetrahydropyran-2-yl]-1H-pyridin-2-one (Int-B5 batch 3, 900 mg, 1.59 mmol, 53% yield) as a solid.

Int-B5 batch 1: LCMS: [M+H]⁺=516.2; Purity=92% (220 nm); Ret. time=0.536 min. ¹H NMR (400 MHz, CDCl₃) δ 7.87 (t, J=7.3 Hz, 1H), 7.66-7.47 (m, 3H), 7.38 (br s, 1H), 6.67-6.53 (m, 1H), 4.67-4.48 (m, 2H), 4.07-3.97 (m, 3H), 3.86-3.70 (m, 1H), 3.60-3.44 (m, 1H), 2.77 (s, 3H), 2.36 (br d, J=12.1 Hz, 1H), 2.27-2.01 (m, 4H).

Int-B5 batch 2: LCMS: [M+H]⁺=516.2; Purity=53% (220 nm); Ret. time=0.539 min.

Int-B5 batch 3: LCMS: [M+H]⁺=516.2; Purity=91% (220 nm); Ret. time=0.527 min.

An analogous procedure was followed to obtain the following intermediates.

| Structure | Starting Material | Characterization |
|---|---|---|
| 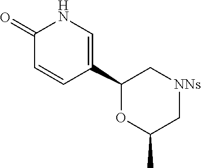 | 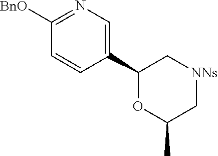 | (2S,6R)-2-(6-benzyloxy-3-pyridyl)- 6-methyl-4-(4-nitrophenyl)sulfonyl- morpholine (Int-C1, 1 equiv., 2.0 g, 4.3 mmol) was added to a mixture of TFA (21 mL) and DCM (21 mL) and the resulting mixture was stirred at 80º C. for 2 h. The reaction mixture was cooled down to r.t. and concentrated under reduced pressure. The crude was purified by flash |
| Int-C10 | Int-C1 | chromatography (80 g SiO₂ column) |
| | | using am elution gradient of 0-4% |
| | | MeOH in DCM to yield 5-((2S,6R)- |
| | | 6-methyl-4-((4- |
| | | nitrophenyl)sulfonyl)morpholin-2- |
| | | yl)pyridine-2(1H)-one (Int-C10, 1.57 |
| | | g, 4.14 mmol, 97%) as a solid. ESI- |
| | | MS: [M + H]⁺ = 381.2. |
| 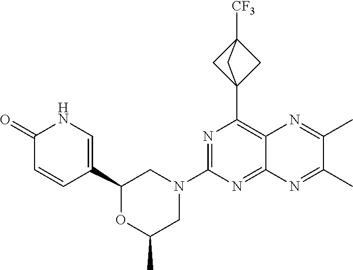 [SMILES: Cc1nc2nc(N3C[C@@H](C)O[C@@H](c4ccc(=O)[nH]c4)C3)nc(C34CC(C(F)(F)F)(C3)C4)c2nc1C] | 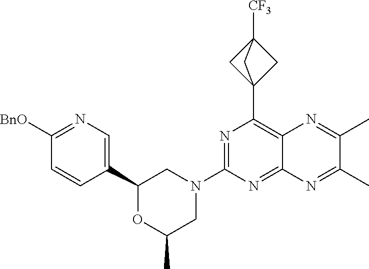 [SMILES: Cc1nc2nc(N3C[C@@H](C)O[C@@H](c4ccc(OCc5ccccc5)nc4)C3)nc(C34CC(C(F)(F)F)(C3)C4)c2nc1C] | Modification: The reaction was performed at 80° C. for 3 h in a sealed MW vial. The reaction mixture was evaporated to dryness and purified by normal phase flash chromatography (24 g SiO₂ column) using an elution gradient of 0-10% MeOH to yield 5- [(2S,6R)-4-[6,7-dimethyl-4-[3- (trifluoromethyl)-1- bicyclo[1.1.1]pentanyl]pteridin-2- yl]-6-methyl-morpholin-2-y1]-1H- pyridin-2-one (Int-C16, 64 mg, 0.13 mmol, 58% yield) as a solid. |
| Int-C16 | Int-C15 | |
| 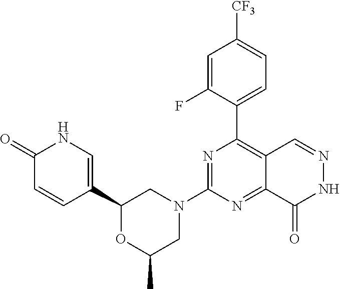 [SMILES: C[C@@H]1CN(c2nc(-c3ccc(C(F)(F)F)cc3F)c3cn[nH]c(=O)c3n2)C[C@H](c2ccc(=O)[nH]c2)O1] | 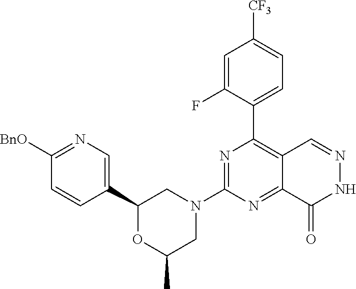 | Modifications: The reaction was run at 80 ° C. for 12 h to obtain 4-[2- fluoro-4-(trifluoromethyl)phenyl]-2- [(2R,6S)-2-methyl-6-(6-oxo-1H- pyridin-3-yl)morpholin-4-y1]-7H- pyrimido[4,5-d]pyridazin-8-one (Int- D25, 90 mg, 0.179 mmol, 62% yield) as a solid. LCMS: [M + H]⁺ = 503.1. |
| Int-D25 | Int-D24 | |
| 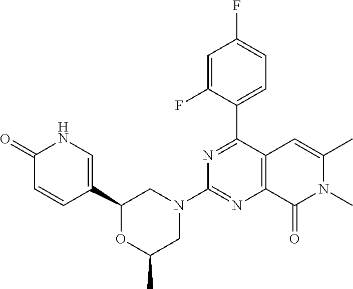 [SMILES: Cc1cc2c(-c3ccc(F)cc3F)nc(N3C[C@@H](C)O[C@@H](c4ccc(=O)[nH]c4)C3)nc2c(=O)n1C] | 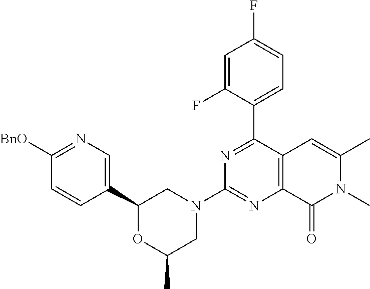 | Modification: Int-D34 was treated with 2:1 mixture of DCE/TFA at 50° C. for 12 h to obtain 4-(2,4- difluorophenyl)-6,7-dimethyl-2- [(2R,6S)-2-methyl-6-(6-oxo-1H- pyridin-3-yl)morpholin-4- yl]pyrido[3,4-d]pyrimidin-8-one (230 mg, 0.480 mmol, 109% yield) as a solid. LCMS: [M + H]⁺ = 480.2. ¹H NMR (400 MHz, DMSO-d₆) δ 7.68- 7.62 (m, 1H), 7.55-7.40 (m, 4H), 7.33-7.27 (m, 1H), 6.34 (d, J = 9.3 Hz, 1H), 6.00 (d, J = 2.6 Hz, 1H), 4.62 (br t, J = 12.4 Hz, 2H), 4.40 (dd, J = 2.3, 10.6 Hz, 1H), 3.78-3.69 (m, 1H), 3.49 (s, 3H), 2.97-2.86 (m, 1H), 2.77-2.64 (m, 1H), 2.31 (s, 3H), 1.22 (br d, J = 5.7 Hz, 3H). |
| Int-D35 | Int-D34 | |
| 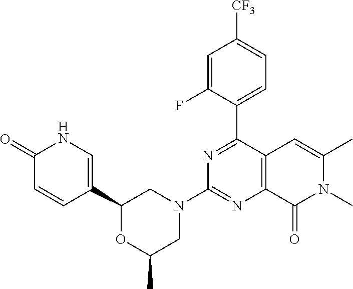 [SMILES: Cc1cc2c(-c3ccc(C(F)(F)F)cc3F)nc(N3C[C@@H](C)O[C@@H](c4ccc(=O)[nH]c4)C3)nc2c(=O)n1C] | 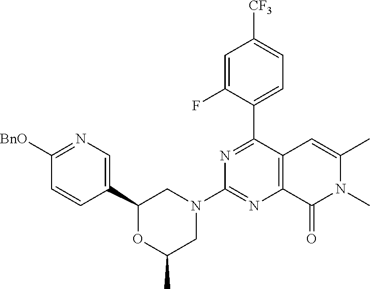 | Modification: Int-D37 was treated with 2:1 mixture of DCE/TFA at 50º C. for 12 h to obtain crude 4-[2- fluoro-4-(trifluoromethyl)phenyl]- 6,7-dimethyl-2-[(2R,6S)-2-methyl- 6-(6-oxo-1H-pyridin-3- yl)morpholin-4-yl]pyrido[3,4- d]pyrimidin-8-one (Int-D38, 200 mg, 0.378 mmol, 156% yield) as a solid. LCMS: [M + H]⁺ = 530.2. ¹H NMR (400 MHz, DMSO-d₆) δ 11.70- 11.49 (m, 1H), 7.93 (br d, J = 9.5 Hz, 1H), 7.89-7.75 (m, 2H), 7.55-7.49 (m, 1H), 7.47-7.40 (m, 2H), 7.35- 7.27 (m, 2H), 7.26-7.15 (m, 1H), 6.35 (br d, J = 9.4 Hz, 1H), 6.03 (br s, 1H), 4.72-4.53 (m, 2H), 4.49 (d, J = 5.4 Hz, 1H), 4.41 (br d, J = 10.8 Hz, |
| | | 1H), 3.80-3.68 (m, 1H), 3.53-3.48 |
| Int-D38 | Int-D37 | (m, 3H), 2.31 (s, 3H), 1.23 (br d, J = |
| | | 5.1 Hz, 4H). |
| 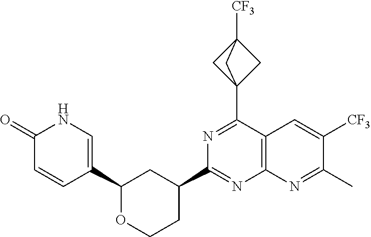 [SMILES: Cc1nc2nc([C@H]3CCO[C@@H](c4ccc(=O)[nH]c4)C3)nc(C34CC(C(F)(F)F)(C3)C4)c2cc1C(F)(F)F] | 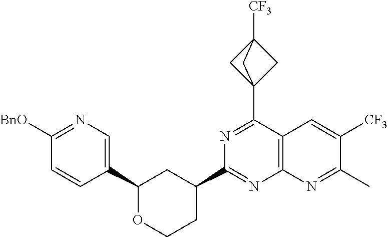 | Modification: The reaction was run at 80° C. for 1 h and purified purified by normal phase flash column chromatography (4 g SiO₂ column) (0-10% MeOH in DCM) to yield 5- [(2R,4S)-4-[7-methyl-6- (trifluoromethyl)-4-[3- (trifluoromethyl)-1- bicyclo[1.1.1]pentanyl]pyrido[2,3- d]pyrimidin-2-yl]tetrahydropyran-2- yl]-1H-pyridin-2-one (Int-E10, 20 mg, 0.04 mmol, 78% yield) as a solid. ESI-MS: [M + H]⁺ = 525.2. ¹H NMR (400 MHz, CD₃OD): δ 8.91 (s, 1H), 7.68 (dd, J = 9.4, 2.5 Hz, 1H), 7.45 |
| | | (d, J = 2.3 Hz, 1H), 6.53 (d, J = 9.6 |
| Int-E10 | Int-E9 | Hz, 1H), 4.44 (dd, J = 11.3, 1.7 Hz, |
| | | 1H), 4.26-4.22 (m, 1H), 3.84-3.77 |
| | | (m, 1H), 3.53-3.45 (m, 1H), 2.89 (s, |
| | | 3H), 2.68 (s, 6H), 2.27 (d, J = 13.3 |
| | | Hz, 1H), 2.10-2.04 (m, 2H), 1.95 (dd, |
| | | J = 24.8, 11.8 Hz, 1H). ¹⁹F NMR |
| | | (376 MHz, CD₃OD): δ −63.7 (s, 3F), |
| | | −74.6 (s, 3F). |
| 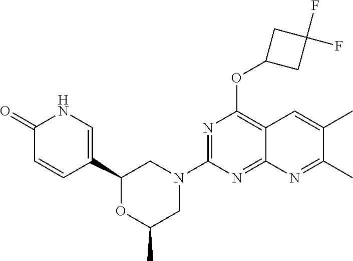 [SMILES: Cc1cc2c(OC3CC(F)(F)C3)nc(N3C[C@@H](C)O[C@@H](c4ccc(=O)[nH]c4)C3)nc2nc1C] | 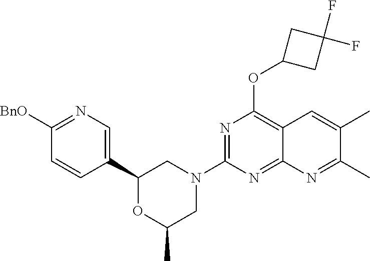 [SMILES: Cc1cc2c(OC3CC(F)(F)C3)nc(N3C[C@@H](C)O[C@@H](c4ccc(OCc5ccccc5)nc4)C3)nc2nc1C] | Modification: The reaction was run at 80° C. for 5 h, and the crude was purified by flash chromatography (Isco RediSep® column 12 g, using a gradient elution of 0-10% MeOH in DCM) to obtain 5-((2S,6R)-4-(4- (3,3-difluorocyclobutoxy)-6,7- dimethylpyrido[2,3-d]pyrimidin-2- yl)-6-methylmorpholine-2- yl)pyridine-2(1H)-one (Int-E14, 47.9 mg, 0.11 mmol, 88%) as a solid. 1H NMR (CDCl₃, 400 MHz): δ 11.7 (br s, 1H), 8.21 (s, 1H), 7.68 (s, 1H), 7.52 (s, 1H), 6.69 (d, J = 9.3 Hz, 1H), 5.32 (br s, 1H), 5.29 (s, 1H), 4.75 (br s, 1H), 4.59 (br s, 1H), 4.34 (d, J = 10.6 |
| | | Hz, 1H), 3.70 (br s, 1H), 3.19 (br s, |
| Int-E14 | Int-E13 | 2H), 2.97-2.83 (m, 2H), 2.79 (s, 3H), |
| | | 2.41 (s, 3H), 1.30 (d, J = 6.1 Hz, 3H), |
| | | 1.25 (s, 1H). ESI-MS: [M + H]⁺ = |
| | | 458.4. |
| 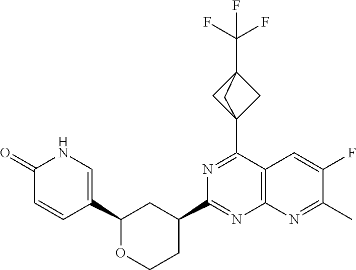 [SMILES: Cc1nc2nc([C@H]3CCO[C@@H](c4ccc(=O)[nH]c4)C3)nc(C34CC(C(F)(F)F)(C3)C4)c2cc1F] | 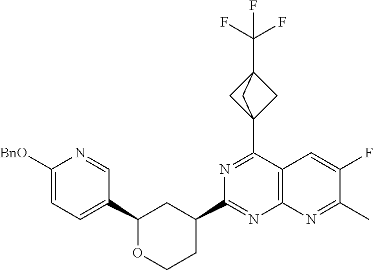 [SMILES: Cc1nc2nc([C@H]3CCO[C@@H](c4ccc(OCc5ccccc5)nc4)C3)nc(C34CC(C(F)(F)F)(C3)C4)c2cc1F] | Modifications: The reaction was run with 100 equiv. of TFA in DCE solvent at 75° C. for 2 h. The crude product was purified by chromatography (40 g SiO₂) using 0- 15% DCM in MeOH gradient elution to afford 5-[(2R,4S)-4-[6-fluoro-7- methyl-4-[3-(trifluoromethyl)-1- bicyclo[1.1.1]pentanyl]pyrido[2,3- d]pyrimidin-2-yl]tetrahydropyran-2- yl]-1H-pyridin-2-one (Int-E24, 167 mg, 0.35 mmol, 99% yield) as a foam. |
| Int-E24 | Int-E23 | |
| 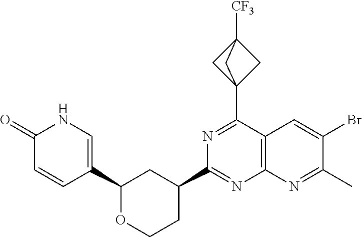 [SMILES: Cc1nc2nc([C@H]3CCO[C@@H](c4ccc(=O)[nH]c4)C3)nc(C34CC(C(F)(F)F)(C3)C4)c2cc1Br] | 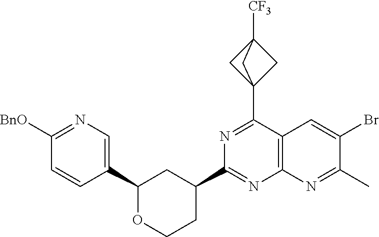 [SMILES: Cc1nc2nc([C@H]3CCO[C@@H](c4ccc(OCc5ccccc5)nc4)C3)nc(C34CC(C(F)(F)F)(C3)C4)c2cc1Br] | The procedure yielded 5-((2R,4S)-4- (6-bromo-7-methyl-4-(3- (trifluoromethyl)bicyclo[1.1.1]pentan- 1-yl)pyrido[2,3-d]pyrimidin-2- yl)tetrahydro-2H-pyran-2- yl)pyridin-2(1H)-one (Int-E31). |
| Int-E31 | Int-E30 | |
| 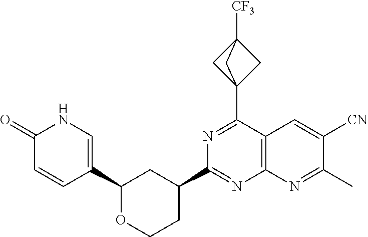 [SMILES: Cc1nc2nc([C@H]3CCO[C@@H](c4ccc(=O)[nH]c4)C3)nc(C34CC(C(F)(F)F)(C3)C4)c2cc1C#N] | 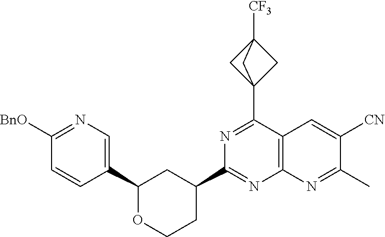 | Modifications: The reaction was run with 100 equiv. of TFA in DCE solvent at 75° C. for 2 h. The crude mixture was purified by normal phase chromatography (0-50% MeOH in DCM elution gradient) to afford 7-methyl-2-((2R,4S)-2-(6- oxo-1,6-dihydropyridin-3- yl)tetrahydro-2H-pyran-4-yl)-4-(3- (trifluoromethyl)bicyclo[1.1.1]pentan- 1-yl)pyrido[2,3-d]pyrimidine-6- carbonitrile (Int-E34, 55 mg, 73% yield) as a solid. ESI-MS: [M + H]⁺ = 482.3. ¹H NMR (CDCl₃, 400 MHz): |
| | | δ 8.81-8.84 (1H, m), 7.77 (1H, d, J = |
| Int-E34 | Int-E33 | 9.0 Hz), 7.61 (1H, s), 7.38 (1H, s), |
| | | 6.78 (1H, d, J = 9.3 Hz), 4.43 (1H, d, |
| | | J = 11.1 Hz), 4.30 (1H, d, J = 11.3 |
| | | Hz), 3.79 (1H, t, J = 12.1 Hz), 3.49 |
| | | (1H, br s), 3.00 (3H, s), 2.65 (6H, s), |
| | | 2.33 (1H, d, J = 13.8 Hz), 2.06-2.13 |
| | | (2H, m), 1.99 (1H, t, J = 12.7 Hz). ¹⁹F |
| | | NMR (CDCl₃, 376 MHz): δ −73.2 |
| | | (3F, s). |
| 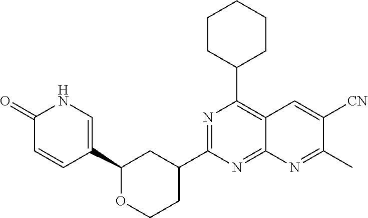 [SMILES: Cc1nc2nc(C3CCO[C@@H](c4ccc(=O)[nH]c4)C3)nc(C3CCCCC3)c2cc1C#N] | 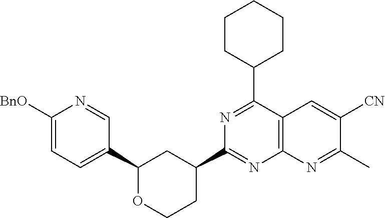 [SMILES: Cc1nc2nc([C@H]3CCO[C@@H](c4ccc(OCc5ccccc5)nc4)C3)nc(C3CCCCC3)c2cc1C#N] | Modifications: The reaction was run with 100 equiv. of TFA in DCE solvent at 75° C. for 2 h. The crude mixture was purified by normal phase chromatography (0-15% MeOH in DCM elution gradient) to afford 4-cyclohexyl-7-methyl-2- [(2R,4S)-2-(6-oxo-1H-pyridin-3- yl)tetrahydropyran-4-yl]pyrido[2,3- d]pyrimidine-6-carbonitrile (Int-E41, 220 mg, 0.51 mmol, 74% yield) as a solid. |
| Int-E41 | Int-E40 | |

### Method Int 50. Intermediate Int-B6:5-methyl-6-oxo-1H-pyridine-3-carbaldehyde

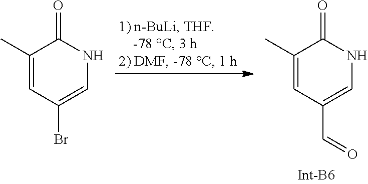

A mixture of 5-bromo-3-methyl-1H-pyridin-2-one (1 equiv., 23 g, 122 mmol) in THF (1150 mL) was degassed with N₂ for 3 times. The mixture was cooled to-78° C. and stirred for 20 min. n-Butyl lithium (3 equiv., 147 mL, 367 mmol) was added to the mixture at −78° C. The mixture was stirred at −78° C. for 3 h. N,N-dimethylformamide (24 equiv., 227 mL, 2936 mmol) was added at −78° C. The mixture was stirred at −70° C. for 1 h. TLC showed the starting material consumed completely and a new point (R_{f}=0.2) was detected. Water (200 mL) was added to the mixture at −70° C. slowly. The mixture was warmed to 25° C. and added saturated sodium chloride solution (100 mL). The mixture was extracted with EtOAc (1 L ×5) and DCM (1 L ×10). The organic layers were added silica gel and concentrated under reduced pressure to give the crude. The crude was purified by silica gel column chromatography (50% EtOAc in PE, 2,4-dinitrophenylhydrazine, R_{f}=0.1) to afford 5-methyl-6-oxo-1H-pyridine-3-carbaldehyde (Int-B6 batch 1, 8.4 g, 61.3 mmol, 50% yield) as a solid and 5-methyl-6-oxo-1H-pyridine-3-carbaldehyde (Int-B6 batch 2, 3.4 g, 24.8 mmol, 20% yield) as a solid.

Int-B6 batch 1: LCMS: [M+H]⁺=138.0; purity=95% (UV 220 nm); Ret. time=0.393 min. ¹H NMR (400 MHz, CDCl₃) δ 9.64 (s, 1H), 7.91-7.77 (m, 2H), 2.20 (s, 3H).

Int-B6 batch 2: LCMS: [M+H]⁺=137.9; purity=64% (UV 220 nm); Ret. time=0.282 min. ¹H NMR (400 MHz, CDCl₃) δ 9.62 (s, 1H), 8.01 (s, 2H), 7.91-7.84 (m, 1H), 7.83-7.73 (m, 1H), 2.95 (s, 5H), 2.88 (s, 5H), 2.18 (s, 3H).

### Method Int 51. Intermediate Int-B7:6-benzyloxy-5-methyl-pyridine-3-carbaldehyde

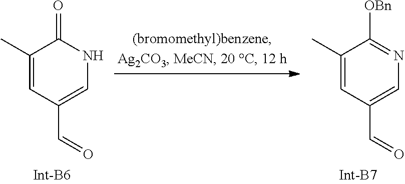

To a mixture of 5-methyl-6-oxo-1H-pyridine-3-carbaldehyde (Int-B6, 1 equiv., 3400 mg, 24.8 mmol) in MeCN (68 mL) was added benzyl bromide (1.1 equiv., 9.5 mL, 27.3 mmol) and Ag₂CO₃ (1.3 equiv., 8887 mg, 32.2 mmol) at 20° C. The mixture was stirred at 20° C. for 12 h. LCMS showed the starting material was consumed completely and a peak with desired mass was detected. The mixture was combined with another batch. The mixture was filtered and washed with EtOAc (500 mL). The filtrate was added silica gel and concentrated under reduced pressure to give the crude. The crude was purified by silica gel column chromatography (PE:EtOAc=10:1; UV, R_{f}=0.6) to afford 6-benzyloxy-5-methyl-pyridine-3-carbaldehyde (Int-B7, 12200 mg, 53.7 mmol, 217% yield) as an oil. LCMS: [M+H]⁺=228.1; purity=98% (UV 220 nm); Ret. time=0.573 min. ¹H NMR (400 MHz, CDCl₃) δ 9.93 (s, 1H), 8.48 (d, J=2.1 Hz, 1H), 7.90 (dd, J=1.0, 2.1 Hz, 1H), 7.52-7.44 (m, 2H), 7.43-7.31 (m, 3H), 5.52 (s, 2H), 2.29 (s, 3H).

### Method Int 52. Intermediate Int-B8: (2R)-2-(6-benzyloxy-5-methyl-3-pyridyl)-2,3-dihydropyran-4-one

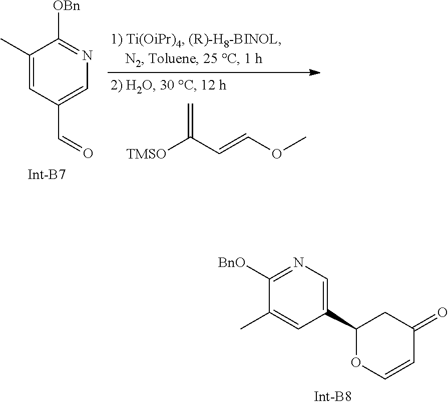

Solution A: To a mixture of Ti(OiPr)₄ (0.4 equiv., 5003 mg, 17.6 mmol) in toluene (30 mL) was added (R)-(+)-5,5,6,6,7,7,8,8-octahydro-1,1-bi-naphthol (0.2 equiv., 2591 mg, 8.80 mmol) at 25° C. The mixture was stirred at 25° C. for 1 h. The mixture was concentrated under reduce pressure at 50° C. to give a solid. The solid was dissolved in toluene (30 mL). To a mixture of 6-benzyloxy-5-methyl-pyridine-3-carbaldehyde (1 equiv., 10.0 g, 44 mmol) in toluene (90 mL) was added trans-1-methoxy-3-(trimethylsilyloxy)-1,3-butadiene (4 equiv., 34 mL, 176 mmol) and H₂O (0.1 equiv., 0.079 mL, 4.4 mmol) at 25° C. The mixture was degassed with N₂ (×3) and the solution A was added at 25° C. The mixture was stirred at 30° C. for 12 h. LCMS showed the starting material was consumed completely and a major peak with desired MS was detected. 200 mL water was added to the mixture and the mixture was extracted with EtOAc (200 mL×6). Silica gel was added to the organic layers and the mixture was concentrated under reduced pressure to give the crude product. The crude product was purified by silica gel column chromatography (25% EtOAc in PE, R_{f}=0.3) to afford (2R)-2-(6-benzyloxy-5-methyl-3-pyridyl)-2,3-dihydropyran-4-one (Int-B8, 3.2 g, 10.1 mmol, 23% yield) as an oil. LCMS: [M+H]⁺=296.3; purity=93% (UV 220 nm); Ret. time=0.579 min. ¹H NMR (400 MHz, CDCl₃) δ 8.02 (d, J=1.7 Hz, 1H), 7.52-7.43 (m, 4H), 7.38 (t, J=7.4 Hz, 2H), 7.33 (br d, J=7.2 Hz, 1H), 5.53 (d, J=6.1 Hz, 1H), 5.43 (s, 2H), 5.37 (dd, J=3.2, 14.5 Hz, 1H), 3.26 (s, 1H), 2.94 (dd, J=14.5, 16.7 Hz, 1H), 2.62 (dd, J=3.2, 16.6 Hz, 1H), 2.27 (s, 3H).

An analogous procedure was followed to obtain the following intermediates.

| Structure | Starting Material | Characterization |
|---|---|---|
| 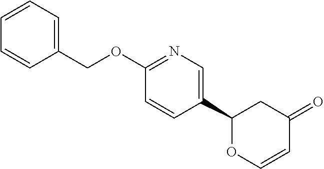 [SMILES: O=C1C=CO[C@@H](c2ccc(OCc3ccccc3)nc2)C1] | 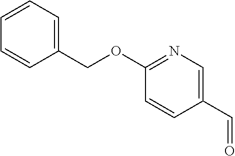 [SMILES: O=Cc1ccc(OCc2ccccc2)nc1] | The reaction was run with 6-benzyloxypyridine- 3-carbaldehyde to produce (R)-2-(6- (benzyloxy)pyridin-3-yl)-2,3-dihydro-4H-pyran- 4-one (Int-F4, 80% yield) as a solid. ¹H NMR (CDCl₃, 400 MHz): δ 8.19 (1H, d, J = 2.5 Hz), 7.64 (1H, dd, J = 8.6, 2.6 Hz), 7.42-7.45 (3H, m), 7.29-7.39 (3H, m), 6.86 (1H, d, J = 8.6 Hz), 5.52 (1H, dd, J = 6.0, 1.3 Hz), 5.36-5.40 (3H, m), 2.91 (1H, dd, J = 16.8, 14.4 Hz), 2.62 (1H, ddd, J = 16.8, 3.4, 1.3 Hz). ESI-MS: [M + H]⁺ = 282.2. |

### Method Int 53. Intermediate Int-B9: (2R)-2-(6-benzyloxy-5-methyl-3-pyridyl)tetrahydropyran-4-one

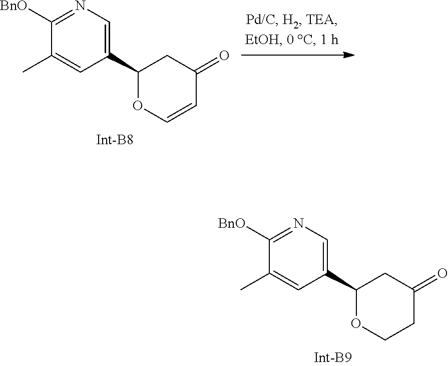 [SMILES: Cc1cc([C@H]2CC(=O)C=CO2)cnc1OCc1ccccc1 ; Cc1cc([C@H]2CC(=O)CCO2)cnc1OCc1ccccc1]

To a mixture of (2R)-2-(6-benzyloxy-5-methyl-3-pyridyl)-2,3-dihydropyran-4-one (1 equiv., 1.5 g, 5.08 mmol) in EtOH (21.5 mL) was added TEA (1 equiv., 2.2 mL, 5.08 mmol) and palladium on carbon (1 equiv., 540 mg, 5.08 mmol) at 25° C. under N₂. The mixture was degassed with H₂ for 3 times and cooled to 0° C. The mixture was stirred at 0° C. for 30 min under H₂ (15 psi) atmosphere. LCMS showed the starting material was consumed completely and a peak with desired MS was detected. The mixture was filtered and washed with EtOH (50 mL). The filtrate was concentrated under reduced pressure to give a crude product. The crude product was purified by reversed-phase flash (0.1% FA condition) and concentrated to remove CH₃CN, then adjusted pH to about 9~10 by using Na₂CO₃ solid, followed by extracted with EtOAc (3×40 mL). The combined organic layers were dried over anhydrous Na₂SO₄, filtered and concentrated under reduced pressure to afford (2R)-2-(6-benzyloxy-5-methyl-3-pyridyl)tetrahydropyran-4-one (Int-B9, 440 mg, 1.48 mmol, 29% yield) as an oil. LCMS: [M+H]⁺=298.3; purity=100% (UV 220 nm); Ret. time=0.580 min. ¹H NMR (400 MHz, CDCl₃) δ 7.98-7.94 (m, 1H), 7.50-7.43 (m, 3H), 7.41-7.28 (m, 3H), 5.44-5.39 (m, 2H), 4.60 (dd, J=3.4, 10.7 Hz, 1H), 4.45-4.37 (m, 1H), 3.84 (dt, J=2.9, 12.0 Hz, 1H), 2.79-2.57 (m, 3H), 2.49-2.39 (m, 1H), 2.26 (s, 3H).

### Method Int 54. Intermediate Int-B10: (5R)-5-(6-benzyloxy-5-methyl-3-pyridyl)-1,6-dioxaspiro[2.5]octane-2-carbonitrile

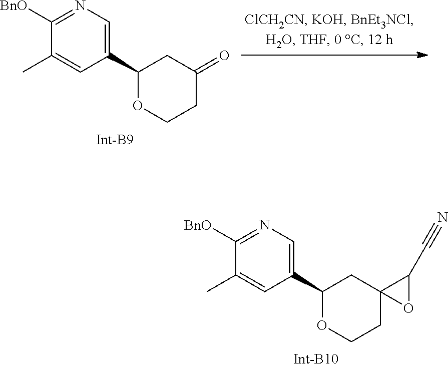 [SMILES: Cc1cc([C@H]2CC(=O)CCO2)cnc1OCc1ccccc1 ; Cc1cc([C@H]2CC3(CCO2)OC3C#N)cnc1OCc1ccccc1]

To a mixture of KOH (3 equiv., 734 mg, 13.1 mmol) in H₂O (9.33 equiv., 0.73 mL, 40.8 mmol) and THF (26 mL) was added BnEt₃NCl (0.05 equiv., 50 mg, 0.219 mmol) at 25° C. The mixture was cooled to 0° C. To the mixture was added (2R)-2-(6-benzyloxy-5-methyl-3-pyridyl)tetrahydropyran-4-one (Int-B9, 1 equiv., 1300 mg, 4.37 mmol) and ClCH₂CN (1.3 equiv., 429 mg, 5.68 mmol) in THF (8.5 mL) at 0° C. The mixture was stirred at 0° C. for 2 h. LCMS showed 12% starting material remained and a main peak with desired mass was detected. The mixture was stirred at 0° C. for another 12 h. LCMS showed 7% starting material remained and a major peak with desired mass was detected. The mixture was stirred at 0° C. for another 2 h. LCMS showed 2% starting material remained and a main peak with desired MS was detected. The reaction was diluted with water (50 mL) and then extracted with EtOAc (50 mL×3). The combined organic layers were washed with brine, dried over Na₂SO₄, filtered, and concentrated under reduced pressure to give crude product (5R)-5-(6-benzyloxy-5-methyl-3-pyridyl)-1,6-dioxaspiro[2.5]octane-2-carbonitrile (Int-B10, 1500 mg, 4.46 mmol, 102% yield) as an oil. The crude product was used to next step without further purification. LCMS: [M+H]⁺=337.3; purity=86% (UV 220 nm); Ret. time=0.612 min.

An analogous procedure was followed to obtain the following intermediates.

| Structure | Starting Material | Characterization |
|---|---|---|
| 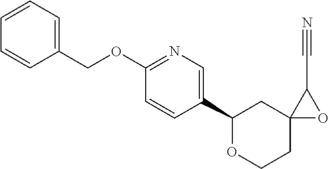 [SMILES: N#CC1OC12CCO[C@@H](c1ccc(OCc3ccccc3)nc1)C2] | 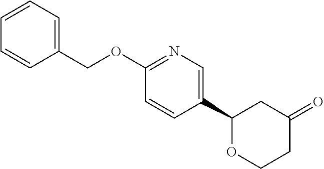 [SMILES: O=C1CCO[C@@H](c2ccc(OCc3ccccc3)nc2)C1] | The procedure afforded (5R)-5-(6-(benzyloxy) pyridin-3-yl)-1,6- dioxaspiro[2.5]octane- 2-carbonitrile (Int-F6). ESI-MS: [M + H]⁺ = 323.2. |
| Int-F6 | Int-F5 | |

### Method Int 55. Intermediate Int-B11: (2R,4S)-2-(6-benzyloxy-5-methyl-3-pyridyl)tetrahydropyran-4-carboxylic acid

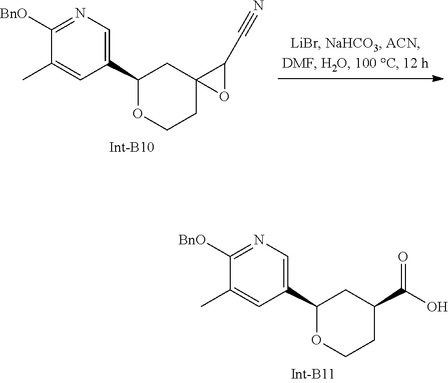 [SMILES: Cc1cc([C@H]2CC3(CCO2)OC3C#N)cnc1OCc1ccccc1 ; Cc1cc([C@H]2C[C@@H](C(=O)O)CCO2)cnc1OCc1ccccc1]

A mixture of LiBr (1.5 equiv., 581 mg, 6.69 mmol), NaHCO₃ (1 equiv., 375 mg, 4.46 mmol) and H₂O (1.25 equiv., 0.10 mL, 5.57 mmol) in DMF (7.5 mL) and MeCN (7.5 mL) was stirred at 25° C. for 15 min. To the mixture was added (5R)-5-(6-benzyloxy-5-methyl-3-pyridyl)-1,6-dioxaspiro[2.5]octane-2-carbonitrile (Int-B10, 1 equiv., 1500 mg, 4.46 mmol) in DMF (7.5 mL) and MeCN (7.5 mL) at 25° C. The mixture was stirred at 100° C. for 12 h. LCMS showed the starting material was consumed completely and a peak with desired mass was detected. The reaction solution was cooled to room temperature and basified with Na₂CO₃ (aq.) to adjust pH ~13, then extracted with EtOAc (3×100 mL). Then the water layer was re-adjusted to pH=2~3 with 1 M HCl aqueous solution and extracted with EtOAc (3×100 mL). The combined organic layers were washed with brine, dried over Na₂SO₄, filtered, and concentrated under reduced pressure to give (2R,4S)-2-(6-benzyloxy-5-methyl-3-pyridyl)tetrahydropyran-4-carboxylic acid (Int-B11, 2.1 g, 6.41 mmol, 144% yield) as an oil. The crude product was used to next step without further purification. LCMS: [M+H]⁺=328.1.

An analogous procedure was followed to obtain the following intermediates.

| Structure | Starting Material | Characterization |
|---|---|---|
| 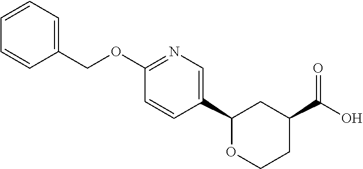 [SMILES: O=C(O)[C@H]1CCO[C@@H](c2ccc(OCc3ccccc3)nc2)C1] | 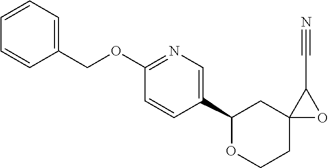 [SMILES: N#CC1OC12CCO[C@@H](c1ccc(OCc3ccccc3)nc1)C2] | Modifications: The reaction was run with 1.5 equiv. Li₂CO₃ instead of NaHCO₃ and at 90° C. overnight to afford (2R,4S)-2-(6- (benzyloxy)pyridin-3- yl)tetrahydro-2H- pyran-4-carboxylic acid (Int-F7, 4.1 g, 78% yield over two steps). ¹H NMR (CD₃OD, 400 MHz): |
| | | δ 8.11 (1H, s), 7.69 (1H, d, |
| Int-F7 | Int-F6 | J = 8.5 Hz), 7.42 (2H, d, |
| | | J = 7.3 Hz), 7.34 (2H, t, J = |
| | | 7.2 Hz), 7.29 (1H, d, J = 7.3 |
| | | Hz), 6.83 (1H, d, J = 8.6 Hz), |
| | | 5.32 (2H, s), 4.38 (1H, d, J = |
| | | 11.5 Hz), 4.13 (1H, d, J = |
| | | 11.5 Hz), 3.64 (1H, t, J = 12.0 |
| | | Hz), 2.73 (1H, t, J = 11.9 |
| | | Hz), 2.08 (1H, d, J = |
| | | 13.4 Hz), 1.90 (1H, d, J = |
| | | 13.4 Hz), 1.75 (1H, |
| | | t, J = 13.0 Hz), 1.63 (1H, |
| | | q, J = 12.4 Hz). ESI- |
| | | MS: [M + H]⁺ = 314.2. |

### Method Int 56. Intermediate Int-B12: (4S)-2-(6-benzyloxy-5-methyl-3-pyridyl)tetrahydropyran-4-carboxamide

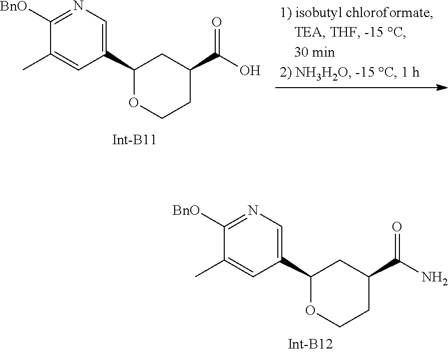 [SMILES: Cc1cc([C@H]2C[C@@H](C(=O)O)CCO2)cnc1OCc1ccccc1 ; Cc1cc([C@H]2C[C@@H](C(N)=O)CCO2)cnc1OCc1ccccc1]

To a solution of (4S)-2-(6-benzyloxy-5-methyl-3-pyridyl)tetrahydropyran-4-carboxylic acid (Int-B11, 1 equiv., 2194 mg, 6.70 mmol) and TEA (2 equiv., 1.9 mL, 13.4 mmol) in THF (21 mL) was added isobutyl i (1.2 equiv., 1.1 mL, 8.04 mmol) drowpise at −15° C. and the mixture was stirred for 30 min. Then NH₃. H₂O (50 equiv., 13 mL, 335 mmol) was added to the mixture and the mixture was stirred for 1 h at −15° C. LCMS showed the starting material was consumed completely and a peak with desired MS was detected. The reaction solution was poured into water (50 mL) and then extracted with EtOAc (3× 50 mL), and the organics was washed with 20 mL saturated brine solution. The combined organic layers were dried over Na₂SO₄, filtered, and concentrated under reduced pressure to give a crude product. The crude product was triturated (50% EtOAc in PE, 20 mL)(×3) to give (4S)-2-(6-benzyloxy-5-methyl-3-pyridyl)tetrahydropyran-4-carboxamide (Int-B12, 1.0 g, 3.06 mmol, 46% yield) as a solid. ¹H NMR (400 MHz, CDCl₃) δ 7.96 (d, J=2.1 Hz, 1H), 7.52-7.44 (m, 3H), 7.42-7.31 (m, 3H), 5.43 (s, 2H), 4.33 (dd, J=2.0, 11.4 Hz, 1H), 4.28-4.21 (m, 1H), 3.66 (br dd, J=11.5, 14.8 Hz, 1H), 2.61 (br s, 1H), 2.26 (s, 3H), 2.04 (td, J=1.9, 11.1 Hz, 1H), 1.96-1.76 (m, 3H).

### Method Int 57. Intermediate Int-B30: (2R,4S)-2-(6-benzyloxy-3-pyridyl)-N-[5-methoxy-6-methyl-3-[3-(trifluoromethyl) bicyclo[1.1.1]pentane-1-carbonyl]pyrazin-2-yl]tetrahydropyran-4-carboxamide

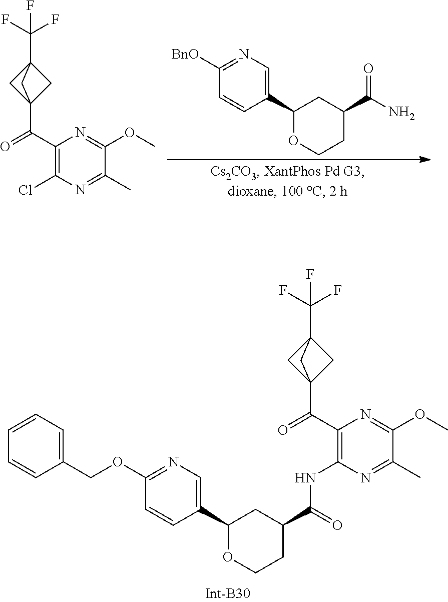 [SMILES: COc1nc(C(=O)C23CC(C(F)(F)F)(C2)C3)c(Cl)nc1C ; NC(=O)[C@H]1CCO[C@@H](c2ccc(OCc3ccccc3)nc2)C1 ; COc1nc(C(=O)C23CC(C(F)(F)F)(C2)C3)c(NC(=O)[C@H]2CCO[C@@H](c3ccc(OCc4ccccc4)nc3)C2)nc1C]

The mixture of (3-chloro-6-methoxy-5-methyl-pyrazin-2-yl)-[3-(trifluoromethyl)-1-bicyclo[1.1.1]pentanyl]methanone (1 equiv., 380 mg, 1.18 mmol), (2R,4S)-2-(6-benzyloxy-3-pyridyl)tetrahydropyran-4-carboxamide (1 equiv., 370 mg, 1.18 mmol), Cs₂CO₃ (2 equiv., 772 mg, 2.37 mmol) and XantPhos Pd G3 (0.1 equiv., 112 mg, 0.118 mmol) in 1,4-dioxane (6.3 mL) was degassed with N₂ for 3 times. The mixture was stirred at 100° C. for 2 h. LCMS showed no starting material remained and a major peak with desired mass was detected. The reaction was combined with another batch, and the final mixture was concentrated under reduced pressure to give a crude. The crude was purified by flash column (100% EtOAc to 9% MeOH in DCM, R_{f}=0.1) to give (2R,4S)-2-(6-benzyloxy-3-pyridyl)-N-[5-methoxy-6-methyl-3-[3-(trifluoromethyl) bicyclo[1.1.1]pentane-1-carbonyl]pyrazin-2-yl]tetrahydropyran-4-carboxamide (Int-B30, 350 mg, 0.587 mmol, 50% yield) as a solid. LCMS: [M+H]⁺=597.2; purity=100% (UV 220 nm); Ret. time=0.658 min. ¹H NMR (400 MHz, CDCl₃) δ 10.97 (s, 1H), 8.15 (d, J=2.3 Hz, 1H), 7.65 (dd, J=2.4, 8.6 Hz, 1H), 7.49-7.41 (m, 2H), 7.40-7.33 (m, 2H), 7.33-7.28 (m, 1H), 6.81 (d, J=8.5 Hz, 1H), 5.38 (s, 2H), 4.47-4.22 (m, 2H), 4.01 (s, 2H), 2.58 (s, 2H), 2.52 (s, 4H), 2.14 (br s, 1H), 2.03-1.96 (m, 2H).

An analogous procedure was followed to obtain the following intermediates.

| Structure | Starting Material | Characterization |
|---|---|---|
| 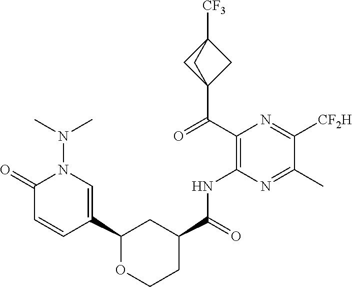 [SMILES: Cc1nc(NC(=O)[C@H]2CCO[C@@H](c3ccc(=O)n(N(C)C)c3)C2)c(C(=O)C23CC(C(F)(F)F)(C2)C3)nc1C(F)F] | 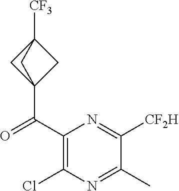 [SMILES: Cc1nc(Cl)c(C(=O)C23CC(C(F)(F)F)(C2)C3)nc1C(F)F] | Modification: The reaction was performed with Int-D16 and 0.8 equiv. of (2R,4S)-2-(1-(dimethylamino)-6- oxo-1,6-dihydropyridin-3- yl)tetrahydro-2H-pyran-4- carboxamide at 85° C. for 30 min. the crude material was purified by flash chromatography (Isco RediSep column 24 g, using a gradient elution of 1-10% MeOH in DCM). The selected fractions were evaporated to yield the desired product (2R,4S)-N- (5-(difluoromethyl)-6-methyl-3-(3- (trifluoromethyl)bicyclo[1.1.1]pentane- 1-carbonyl)pyrazin-2-y1)-2-(1- (dimethylamino)-6-oxo-1,6- dihydropyridin-3-yl)tetrahydro-2H- |
| | | pyran-4-carboxamide (Int-D17, 17 |
| Int-D17 | Int-D16 | mg, 0.03 mmol, 11% yield). ESI-MS: |
| | | [M + H]⁺ = 570.3. |
| | 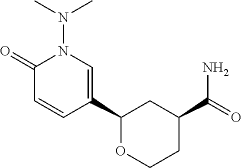 [SMILES: CN(C)n1cc([C@H]2C[C@@H](C(N)=O)CCO2)ccc1=O] | |
| 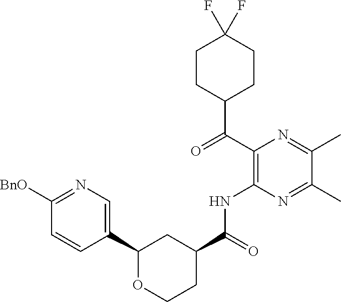 [SMILES: Cc1nc(NC(=O)[C@H]2CCO[C@@H](c3ccc(OCc4ccccc4)nc3)C2)c(C(=O)C2CCC(F)(F)CC2)nc1C] | 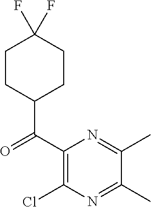 | The crude was then purified by silica gel column (PE/EtOAc= 1:1, Rf=0.5) to give (2R,4S)-2-(6-benzyloxy-3- pyridyl)-N-[3-(4,4- difluorocyclohexanecarbonyl)-5,6- dimethyl-pyrazin-2- yl]tetrahydropyran-4-carboxamide (Int-D41, 3000 mg, 4.93 mmol, 95% yield) as a solid. LCMS: [M + H]⁺ = 565.2. ¹H NMR (400 MHz, CDCl₃) δ 1.79-2.03 (m, 9H) 2.12-2.28 (m, 3H) 2.52-2.73 (m, 6H) 2.93-3.07 (m, 1H) 3.62-3.79 (m, 1H) 3.99-4.09 (m, 1H) 4.24-4.33 (m, 1H) 4.41 (dd, J = 11.3, 1.8 Hz, 1H) 5.38 (s, 2H) 6.82 (d, J = 8.6 Hz, 1H) 7.28-7.41 (m, 3H) 7.46 (d, J = 7.2 Hz, 2H) 7.66 (dd, J = 8.6, 2.4 Hz, 1H) 8.16 (d, J = 2.2 Hz, 1H) 11.28 (s, 1H). |
| Int-D41 | Int-D40 | |
| | 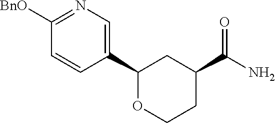 [SMILES: NC(=O)[C@H]1CCO[C@@H](c2ccc(OCc3ccccc3)nc2)C1] | |
| 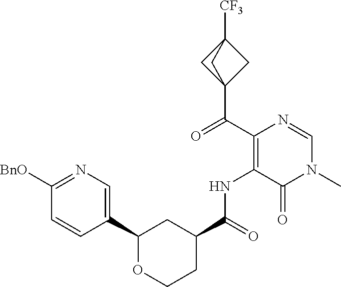 [SMILES: Cn1cnc(C(=O)C23CC(C(F)(F)F)(C2)C3)c(NC(=O)[C@H]2CCO[C@@H](c3ccc(OCc4ccccc4)nc3)C2)c1=O] | 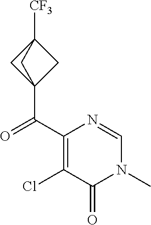 | Purification by SiO₂ flash column chromatography using an elution gradient of 10-45% acetone in hexanes yield methyl (2R,4S)-2-(6- (benzyloxy)pyridin-3-y1)-N-(2- methyl-3-oxo-5-(3- (trifluoromethyl)bicyclo[1.1.1]pentane- 1-carbonyl)-2,3-dihydropyridazin- 4-yl)tetrahydro-2H-pyran-4- carboxamide (Int-D45, 97 mg, 0.17 mmol, 8% yield) as a foam. ESI-MS: [M + H]⁺ = 583.3. ¹H NMR (400 MHz, CDCl₃): δ 8.76 (s, 1H), 8.14 (d, J = 2.3 Hz, 1H), 7.66-7.63 (m, 2H), 7.44 (d, J = 6.9 Hz, 2H), 7.38-7.29 (m, 3H), 6.82 (d, J = 8.5 Hz, 1H), 5.39 (s, 2H), 4.38- 4.35 (m, 1H), 4.26-4.22 (m, 1H), 3.83 |
| | | (s, 3H), 3.68-3.62 (m, 1H), 2.83-2.77 |
| Int-D45 | Int-D44 | (m, 1H), 2.29 (s, 6H), 2.03 (d, J = 13.5 |
| | | Hz, 1H), 1.92-1.86 (m, 2H), 1.77 (dd, |
| | 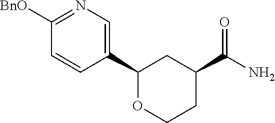 [SMILES: NC(=O)[C@H]1CCO[C@@H](c2ccc(OCc3ccccc3)nc2)C1] | J = 24.8, 12.0 Hz, 1H). |
| 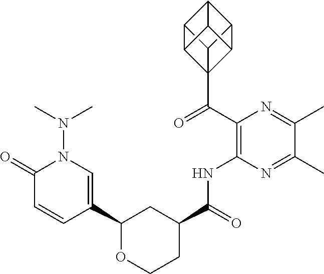 | 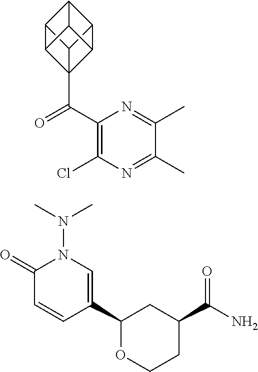 | Modifications: The reaction was run with (3-chloro-5,6-dimethyl-pyrazin- 2-yl)-[cuban-1-yl]methanone (1 equiv.) and (2R,4S)-2-(1- (dimethylamino)-6-oxo-1,6- dihydropyridin-3-yl)tetrahydro-2H- pyran-4-carboxamide (1.5 equiv.) at 85° C. for 2.5 h. The crude was purified by silica gel flash column chromatography, using an elution gradient of 5-45% acetone in hexanes to afford (2R,4S)-N-(3-(cubane-1- carbonyl)-5,6-dimethylpyrazin-2-yl)- 2-(1-(dimethylamino)-6-oxo-1,6- dihydropyridin-3-yl)tetrahydro-2H- pyran-4-carboxamide (Int-D46, 62 mg, 0.123 mmol, 41% yield) as a solid. ESI-MS: [M + H]⁺ = 472.4. 1H NMR (400 MHz, DMSO-d₆): δ 10.70 (d, J = 9.1 Hz, 1H), 7.50 (d, J = 2.3 Hz, 1H), 7.37 (dd, J = 9.4, 2.5 Hz, 1H), 6.34 (d, J = 9.4 Hz, 1H), 4.29-4.26 (m, 3H), 4.16 (d, J = 10.1 Hz, 1H), 4.03- |
| Int-D46 | | 3.94 (m, 5H), 3.52-3.46 (m, 1H), 3.29 |
| | | (s, 3H), 2.87 (d, J = 13.5 Hz, 6H), 2.43 |
| | | (s, 3H), 1.88 (d, J = 13.0 Hz, 1H), 1.74 |
| | | (d, J = 12.6 Hz, 1H), 1.63-1.47 (m, |
| | | 2H). |
| 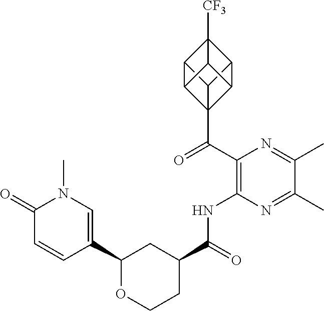 [SMILES: Cc1nc(NC(=O)[C@H]2CCO[C@@H](c3ccc(=O)n(C)c3)C2)c(C(=O)C23C4C5C2C2C3C4C52C(F)(F)F)nc1C] | 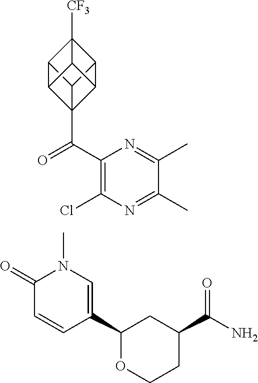 | The crude was purified by silica gel flash column chromatography, using an elution gradient of 10-60% acetone in hexanes to yield (2R,4S)-N-(5,6- dimethyl-3-(4- (trifluoromethyl)cubane-1- carbonyl)pyrazin-2-yl)-2-(1-methyl- 6-oxo-1,6-dihydropyridin-3- yl)tetrahydro-2H-pyran-4- carboxamide (Int-D47, 136 mg, 0.25 mmol, 63% yield) as a solid. ESI-MS: [M + H]⁺ = 541.4. 1H NMR (400 MHz, CDCl₃): δ 11.22 (s, 1H), 7.33 (dd, J = 7.0, 2.6 Hz, 2H), 6.56 (q, J = 3.4 Hz, 1H), 4.40 (t, J = 4.8 Hz, 3H), 4.27 (t, J = 4.6 Hz, 3H), 4.24-4.16 (m, 2H), 3.64 (td, J = 11.5, 3.4 Hz, 1H), 3.53 (s, 3H), 2.92 (t, J = 11.7 Hz, 1H), 2.62 (t, J = 3.5 Hz, 3H), 2.50 (s, 3H), 2.14 (d, J = 15.8 Hz, 1H), 2.00-1.92 (m, 2H), 1.80 (dd, J = 24.8, 12.0 Hz, 1H). |
| Int-D47 | | |

### Method Int 58. Intermediate Int-B13:2-[(2R,4S)-2-(6-benzyloxy-3-pyridyl)tetrahydropyran-4-yl]-6-methoxy-7-methyl-4-[3-(trifluoromethyl)-1-bicyclo[1.1.1]-pentanyl]pteridine

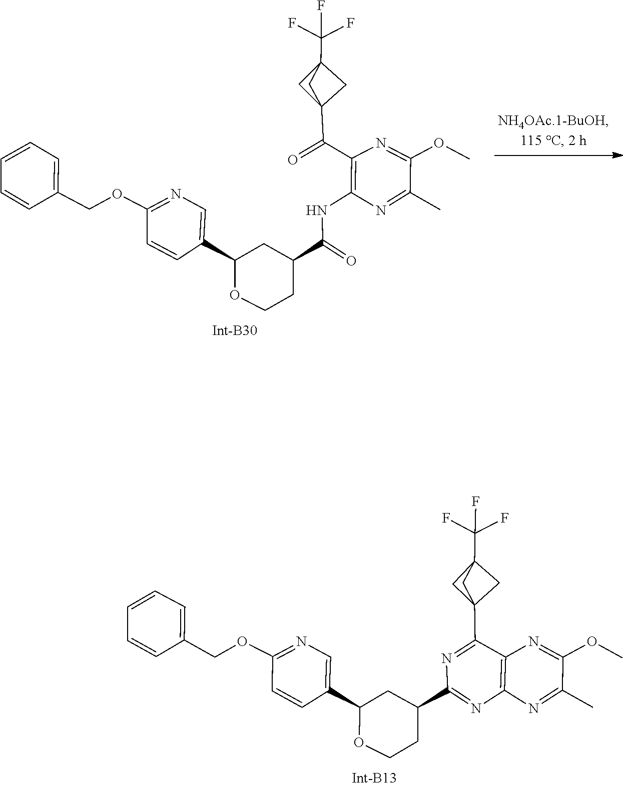 [SMILES: COc1nc(C(=O)C23CC(C(F)(F)F)(C2)C3)c(NC(=O)[C@H]2CCO[C@@H](c3ccc(OCc4ccccc4)nc3)C2)nc1C ; COc1nc2c(C34CC(C(F)(F)F)(C3)C4)nc([C@H]3CCO[C@@H](c4ccc(OCc5ccccc5)nc4)C3)nc2nc1C]

To a solution of (2R,4S)-2-(6-benzyloxy-3-pyridyl)-N-[5-methoxy-6-methyl-3-[3-(trifluoromethyl) bicyclo[1.1.1]pentane-1-carbonyl]pyrazin-2-yl]tetrahydropyran-4-carboxamide (Int-B30, 1 equiv., 300 mg, 0.503 mmol) in 1-butanol (15 mL) was added ammonium acetate (20 equiv., 775 mg, 10.1 mmol) and the mixture was stirred at 115° C. for 2 h. LCMS showed starting material consumed completely and a major peak with desired mass was detected. The mixture was combined with another batch, added to water (50 mL) and extracted with EtOAc (3×50 mL). The combined organic phase was concentrated under reduced pressure to give 2-[(2R,4S)-2-(6-benzyloxy-3-pyridyl)tetrahydropyran-4-yl]-6-methoxy-7-methyl-4-[3-(trifluoromethyl)-1-bicyclo[1.1.1]-pentanyl]pteridine (Int-B13, 360 mg, 0.623 mmol, 124% yield) as a solid, which was used in next step directly. LCMS: [M+H]⁺=578.3; purity=83% (UV 220 nm); Ret. time=0.694 min.

### Method Int 59. Intermediate Int-B14: (5-(4-bromotetrahydropyran-2-yl)-2-chloro-pyrimidine), Int-B15:2-bromo-5-(4-bromotetrahydropyran-2-yl)pyrimidine

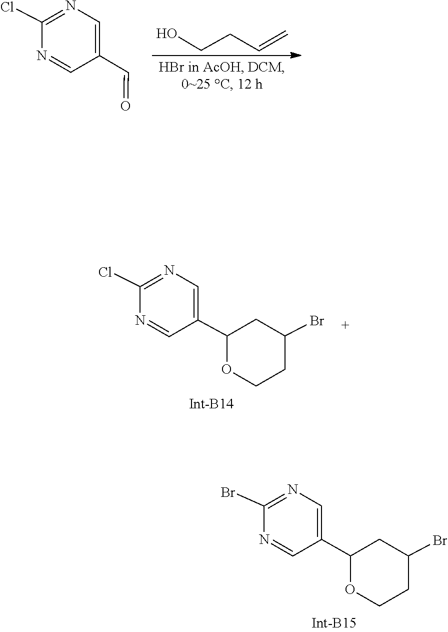

To a mixture of 2-chloropyrimidine-5-carbaldehyde (1 equiv., 2.0 g, 14.0 mmol) and 3-buten-1-ol (1.2 equiv., 1.4 mL, 16.8 mmol) in DCM (80 mL) was added dropwise HBr in AcOH (3 equiv., 7.5 mL, 42.1 mmol) at 0° C. under N₂ atmosphere, then the reaction mixture was stirred at 0° C. for 2 h under N₂ atmosphere. LCMS showed 76% of desired product and by-product. The reaction mixture was poured into saturated NaHCO₃ aqueous solution (100 mL) and then extracted with DCM (100 mL×3). The combined organic layers were dried over Na₂SO₄, filtered, and concentrated under reduced pressure to give a residue (4 g). The residue was purified by silica gel column chromatography (25% EtOAc in petroleum ether, R_{f}=0.5) to afford mixture of 5-(4-bromotetrahydropyran-2-yl)-2-chloro-pyrimidine (Int-B14) & 2-bromo-5-(4-bromotetrahydropyran-2-yl)pyrimidine (Int-B15)(3.36 g) as an inseparable oil that was used in the next step without further purification.

### Intermediate Int-B16:2-benzyloxy-5-(4-bromotetrahydropyran-2-yl)pyrimidine

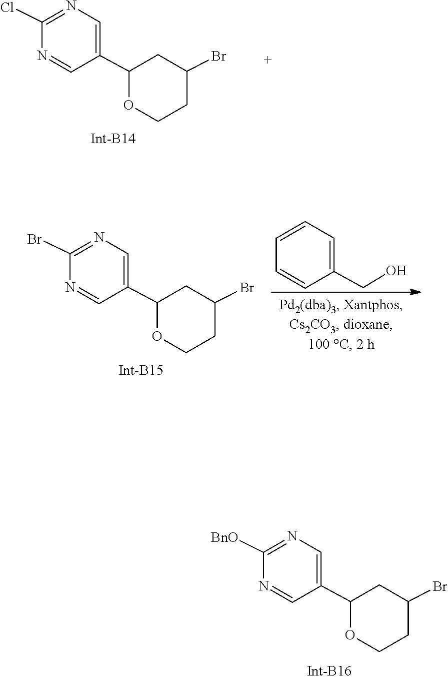

To a mixture of (5-(4-bromotetrahydropyran-2-yl)-2-chloro-pyrimidine), int-B14, and 2-bromo-5-(4-bromotetrahydropyran-2-yl)pyrimidine, Int-B15, (1 equiv., 2400 mg, 5.66 mmol) and phenylmethanol (1.3 equiv., 0.77 mL, 7.36 mmol) in 1,4-dioxane (25 mL) was added Pd₂(dba)₃ (0.1 equiv., 519 mg, 0.566 mmol), XantPhos (0.2 equiv., 656 mg, 1.13 mmol) and Cs₂CO₃ (2.5 equiv., 4614 mg, 14.2 mmol) under N₂ atmosphere. Then the reaction mixture was purged with nitrogen 3 times and was then stirred at 100° C. for 8 hours under nitrogen. LCMS showed 46% of desired product. The reaction was diluted with water (100 mL) and then extracted with EtOAc (3×150 mL). The combined organic layers were washed with brine, dried over Na₂SO₄, filtered, and concentrated under reduced pressure to give a residue. The residue was purified by silica gel column chromatography (20% EtOAc in petroleum ether)(TLC, 9% EtOAc in PE, R_{f}=0.60) to afford 2-benzyloxy-5-(4-bromotetrahydropyran-2-yl)pyrimidine (Int-B16, 1.36 g, 3.89 mmol, 69% yield) as a solid. LCMS: [M+H]⁺=351.0; purity=100% (220 nm); Ret. time=0.572 min. ¹H NMR (400 MHz, CDCl₃, 301 K) δ] ppm] 8.50 (s, 2H), 7.48 (d, J=7.1 Hz, 2H), 7.41-7.29 (m, 3H), 5.46 (s, 2H), 4.34 (dd, J=2.0, 11.4 Hz, 1H), 4.26 (tt, J=4.5, 11.9 Hz, 1H), 4.15 (ddd, J=1.6, 4.9, 12.0 Hz, 1H), 3.60 (dt, J=2.3, 12.1 Hz, 1H), 2.47 (tdd, J=2.0, 4.2, 12.9 Hz, 1H), 2.32-2.25 (m, 1H), 2.18 (dt, J=4.8, 12.4 Hz, 1H), 2.13-2.01 (m, 1H).

### Method Int 60. Intermediate Int-B17: [6-methoxy-7-methyl-2-[(2R)-2-(1-methyl-6-oxo-3-pyridyl)-tetrahydropyran-4-yl]pteridin-4-yl] 4-methylbenzenesulfonate

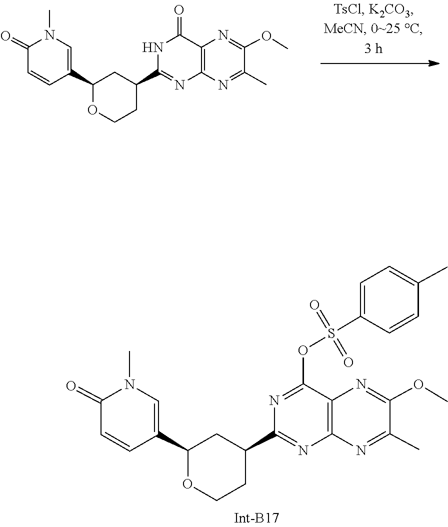 [SMILES: COc1nc2c(=O)[nH]c([C@H]3CCO[C@@H](c4ccc(=O)n(C)c4)C3)nc2nc1C ; COc1nc2c(OS(=O)(=O)c3ccc(C)cc3)nc([C@H]3CCO[C@@H](c4ccc(=O)n(C)c4)C3)nc2nc1C]

To a solution of 6-methoxy-7-methyl-2-[(2R,4S)-2-(1-methyl-6-oxo-3-pyridyl)tetrahydropyran-4-yl]-3H-pteridin-4-one (1 equiv., 30 mg, 0.0782 mmol) and K₂CO₃ (5 equiv., 54 mg, 0.391 mmol) in MeCN (1 mL), then 4-methylbenzenesulfonyl chloride (2 equiv., 30 mg, 0.156 mmol) in MeCN (0.5 mL) was added to the mixture at 0° C. and the solution was stirred for 3 h at 25° C. LCMS showed raw material consumed most and the major new peak showed desired MS. The solution extracted with EtOAc (2×5 mL), the organics washed with saturated brine (5 mL). The combined organic layers were dried over Na₂SO₄, filtered, and concentrated under reduced pressure to give the residue [6-methoxy-7-methyl-2-[(2R)-2-(1-methyl-6-oxo-3-pyridyl)-tetrahydropyran-4-yl]pteridin-4-yl] 4-methylbenzenesulfonate (Int-B17, 30 mg, 0.056 mmol, 71% yield) and use for next step directly. LCMS: [M+H]⁺=538.1, purity=45%, uv=220 nm, Ret. Time=0.560 min.

### Method Int 61. Intermediate Int-B19:5-[(2R,4S)-4-(4-bromo-6-methoxy-7-methyl-pteridin-2-yl)tetrahydropyran-2-yl]-1-methyl-pyridin-2-one

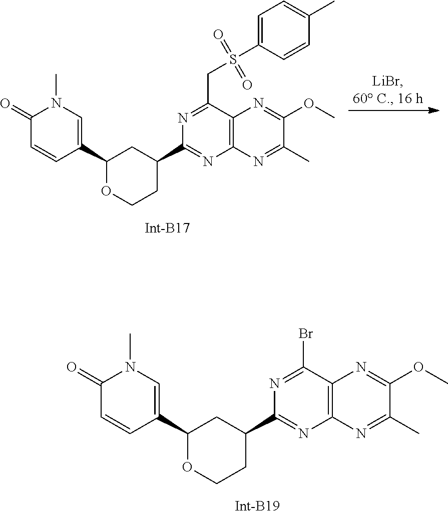 [SMILES: COc1nc2c(CS(=O)(=O)c3ccc(C)cc3)nc([C@H]3CCO[C@@H](c4ccc(=O)n(C)c4)C3)nc2nc1C ; COc1nc2c(Br)nc([C@H]3CCO[C@@H](c4ccc(=O)n(C)c4)C3)nc2nc1C]

LiBr (3.5 equiv., 187 mg, 2.15 mmol) was added to a solution of [6-methoxy-7-methyl-2-[(2R,4S)-2-(1-methyl-6-oxo-3-pyridyl)tetrahydropyran-4-yl]pteridin-4-yl] 4-methylbenzene-sulfonate (Int-B17, 1 equiv., 330 mg, 0.614 mmol) in THF (6 mL). The resulting mixture was stirred for 16 h at 60° C. under N₂. LCMS showed that the starting material was not consumed completely, and the desired mass was detected (72%). The reaction was extracted with EtOAc (10 mL×3), the combined organic phase washed with brine (30 mL), dried over Na₂SO₄, filtered, and concentrated under reduced pressure to give a residue. The residue was purified by prep-TLC (8% MeOH in EtOAc, desired product R_{f}=0.5) to afford 5-[(2R,4S)-4-(4-bromo-6-methoxy-7-methyl-pteridin-2-yl)tetrahydropyran-2-yl]-1-methyl-pyridin-2-one (Int-B19, 170 mg, 0.373 mmol, 61% yield) as an oil. LCMS: [M+H]⁺=448.1; Purity=98% (220 nm); Ret. time=0.493 min.

### Method Int 62. Intermediate Int-B20: bromo-[2-(1-cyclopropyl-6-oxo-3-pyridyl)tetrahydropyran-4-yl]zinc

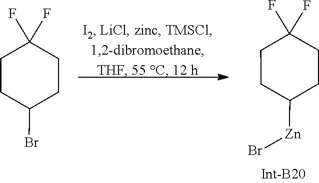

Zinc (3.04 equiv., 200 mg, 3.06 mmol) was suspended in LiCl (0.5 M in THF)(1 equiv., 2.0 mL, 1 mmol), 1,2-dibromoethane (0.05 equiv., 0.004 mL, 0.05 mmol) was added and the suspension was stirred at 55° C. for 20 min, Cooled down, then TMSCl (0.05 equiv., 0.006 mL, 0.05 mmol) was introduced and the mixture was stirred at 55° C. for additional 20 min. Cooled down, then iodine (0.02 equiv., 5.1 mg, 0.0201 mmol) in THF (0.1 mL) was introduced and the reaction was stirred at 55° C. for another 20 min, 4-bromo-1,1-difluoro-cyclohexane (1 equiv., 200 mg, 1 mmol) was added to the warm suspension of activated zinc. The reaction mixture was stirred at 55° C. for 12 h. The solution was used to the next step directly.

### Method Int 63. Intermediate Int-B22:5-[4-(8-chloro-2,3-dimethyl-pyrido[2,3-b]pyrazin-6-yl)tetrahydropyran-2-yl]-1-methyl-pyridin-2-one

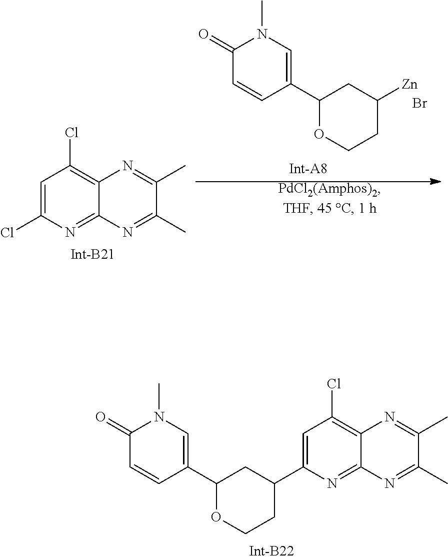

Charged 6,8-dichloro-2,3-dimethyl-pyrido[2,3-b]pyrazine (1 equiv., 130 mg, 0.57 mmol) and Pd(Amphos)₂Cl₂ (0.05 equiv., 20 mg, 0.0285 mmol) and THF (1 mL) into sealed bottle under N₂ atmosphere and purged with N₂ for three times, bromo-[2-(1-methyl-6-oxo-3-pyridyl)tetrahydropyran-4-yl]zinc (1.2 equiv., 231 mg, 0.684 mmol) was added dropwise to the reaction solution at 25° C., then warm to 45° C. and stirred for 1 hour. LCMS showed ~35% of desired product was detected. The mixture was combined with two additional batches, and was quenched by 100 mL H₂O, extracted with DCM (3×100 mL), the combined organic layers was dried over anhydrous Na₂SO₄, filtered, and concentrated under reduced pressure to give the crude product (450 mg). The crude product was purified by silica gel column chromatography (0-100% EtOAc in petroleum ether, and 100-70% EtOAc in MeOH)(TLC, 25% MeOH in EtOAc, desired product: R_{f}=0.6) to give 5-[4-(8-chloro-2,3-dimethyl-pyrido[2,3-b]pyrazin-6-yl)tetrahydropyran-2-yl]-1-methyl-pyridin-2-one (Int-B22, 250 mg, 0.650 mmol, 56% yield) as a solid. LCMS: [M+H]⁺=385.2; purity=96% (UV 220 nm); Ret. time=0.451 min. ¹H NMR (400 MHz, CDCl₃) δ 7.73-7.61 (m, 1H), 7.47-7.32 (m, 2H), 6.63-6.50 (m, 1H), 4.36-4.21 (m, 1H), 4.01-3.86 (m, 1H), 3.76 (dt, J=2.3, 11.8 Hz, 1H), 3.54 (s, 3H), 3.30 (tdd, J=3.9, 8.0, 11.9 Hz, 1H), 2.88-2.76 (m, 6H), 2.34-2.13 (m, 2H), 2.11-2.05 (m, 1H), 2.03-1.97 (m, 1H).

### Method Int 64. Intermediate Int-C12: 1-cyclopropyl-5-((2S,6R)-6-methylmorpholin-2-yl)pyridine-2(1H)-one

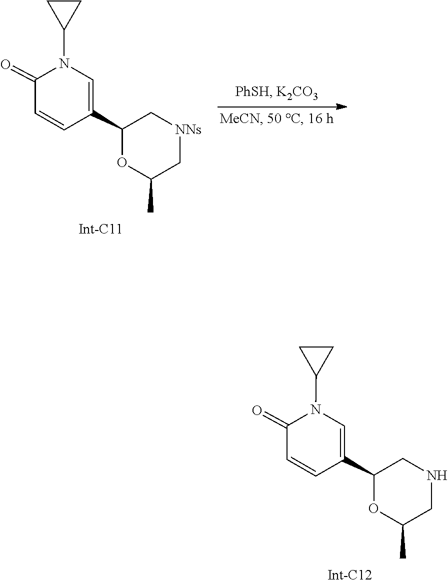

Thiophenol (3 equiv., 36 μL, 0.36 mmol) was added dropwise to a suspension of 1-cyclopropyl-5-[(2S,6R)-6-methyl-4-(4-nitrophenyl) sulfonyl-morpholin-2-yl]pyridin-2-one 4 (Int-C11, 1 equiv., 50.0 mg, 0.12 mmol) and potassium carbonate (5 equiv., 82.3 mg, 0.60 mmol) in MeCN (1.0 mL) at 50° C. under argon atmosphere. The resulting mixture was stirred at 50° C. for 18 h and then cooled to room temperature and filtered through a pad of Celite. The filtrate was concentrated to dryness and the residue was diluted with DCM (100 mL) and extracted with 3M HCl (aq)(3× 20 mL). The combined aqueous layers were basified with 1M NaOH (aq) until pH=12 and extracted with DCM (3×50 mL). The combined organic layers were washed with brine (50 mL), dried over anhydrous Na₂SO₄, filtered, and concentrated under reduced pressure. The crude material was purified by flash chromatography (12 g SiO₂ column) using an elution gradient of 0-10% MeOH in DCM to yield 1-cyclopropyl-5-[(2S,6R)-6-methylmorpholin-2-yl]pyridin-2-one (Int-C12, 20 mg, 0.08 mmol, 72% yield) as an oil. ¹H NMR (CDCl₃, 400 MHz): δ 7.29 (d, J=6.9 Hz, 2H), 6.52 (d, J=10.0 Hz, 1H), 4.29 (d, J=10.2 Hz, 1H), 3.74 (s, 1H), 3.26-3.32 (m, 1H), 2.95 (br s, 2H), 2.58 (br d, J=58.5 Hz, 3H), 1.19 (d, J=6.2 Hz, 3H), 1.11-1.14 (m, 2H), 0.85-0.87 (m, 2H).

An analogous procedure was followed to obtain the following intermediates.

| Structure | Starting Material | Characterization |
|---|---|---|
| 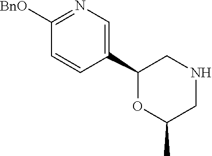 [SMILES: C[C@@H]1CNC[C@H](c2ccc(OCc3ccccc3)nc2)O1] | 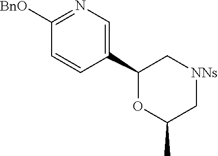 | Modification: The reaction crude was purified by normal phase flash column chromatography (25 g Si02 column) using an elution gradient of 1-10% MeOH in DCM to yield (2S,6R)-2-(6- (benzyloxy)pyridin-3-yl)-6-methylmorpholine (Int- C1, 79%) as a cloudy oil. ¹H NMR (CDCl₃, 400 MHz): δ 8.15 (1H, d, J = 2.3 Hz), 7.63 (1H, dd, J = 8.5, 2.4 Hz), 7.45 (2H, d, J = 7.5 Hz), 7.29-7.39 (3H, m), 6.80 (1H, d, J = 8.5 Hz), 5.38 (2H, s), 4.50 (1H, dd, J = 10.5, 2.3 Hz), 3.75-3.79 (1H, m), 2.97 (2H, dd, J = |
| | | 25.2, 12.3 Hz), 2.71 (1H, t, J = 11.4 Hz), 2.56 (1H, t, |
| Int-C1 | (2S,6R)-2-(6- | J = 11.3 Hz), 2.10-2.14 (1H, m), 1.21 (3H, d, J = 6.2 |
| | benzyloxy-3-pyridyl)- | Hz). |
| | 6-methyl-4-(4- | |
| | nitrophenyl)sulfonyl- | |
| | morpholine | |

### Method Int 66. Intermediate Int-C3:5-((2S,6R)-2-(6-(benzyloxy)pyridin-3-yl)-6-methylmorpholino)-7-(2,4-difluorophenyl)-N,N-dimethylthiazolo[4,5-d]pyrimidin-2-amine

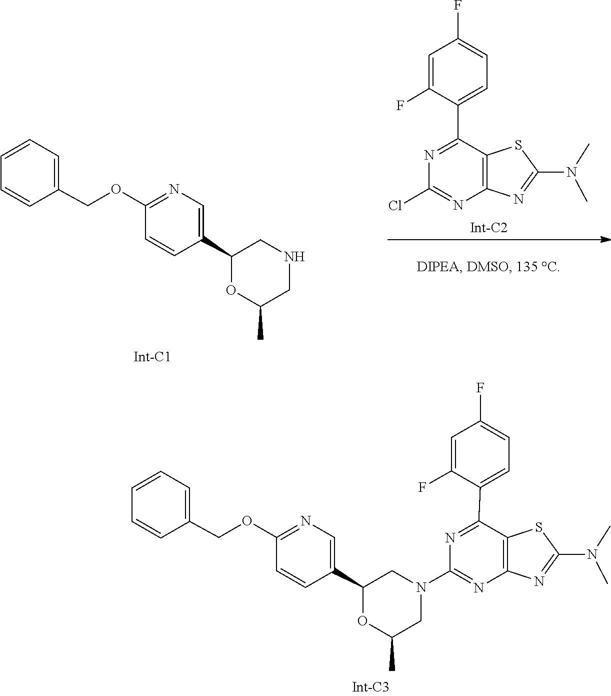 [SMILES: C[C@@H]1CNC[C@H](c2ccc(OCc3ccccc3)nc2)O1 ; CN(C)c1nc2nc(Cl)nc(-c3ccc(F)cc3F)c2s1 ; C[C@@H]1CN(c2nc(-c3ccc(F)cc3F)c3sc(N(C)C)nc3n2)C[C@H](c2ccc(OCc3ccccc3)nc2)O1]

To a solution of (2S,6R)-2-(6-benzyloxy-3-pyridyl)-6-methyl-morpholine (Int-C1, 1 equiv., 115 mg, 0.404 mmol) and 5-chloro-7-(2,4-difluorophenyl)-N,N-dimethyl-thiazolo[4,5-d]pyrimidin-2-amine (Int-C2, 1.2 equiv., 159 mg, 0.485 mmol) in DMSO (2 mL) was added DIPEA (3 equiv., 0.21 mL, 1.21 mmol), and then the mixture was stirred for 4 h at 135° C. More 5-chloro-7-(2,4-difluorophenyl)-N,N-dimethyl-thiazolo[4,5-d]pyrimidin-2-amine (Int-C2, 0.5 equiv., 66 mg, 0.202 mmol) was added to the mixture and then the mixture was stirred for another 12 h at 135° C. The reaction solution was extracted with ethyl acetate (10 mL×2) and washed with 10 mL saturated brine solution. The organics were then separated and dried (Na₂SO₄) before concentration to dryness. The residue was then purified by silica gel column (50% EtOAc in PE, R_{f}=0.5) to obtain 5-[(2S,6R)-2-(6-benzyloxy-3-pyridyl)-6-methyl-morpholin-4-yl]-7-(2,4-difluorophenyl)-N,N-dimethyl-thiazolo[4,5-d]pyrimidin-2-amine (Int-C3) as a gum. LCMS: [M+H]⁺=575.2. ¹H NMR (400 MHz, CDCl₃) δ 1.3 (d, J=6.1 Hz, 3H) 2.76 (d, J=13.3, 10.7 Hz, 1H) 2.90 (dd, J=13.1, 11.3 Hz, 1H) 3.20-3.37 (m, 6H) 3.78-3.92 (m, 1H) 4.59 (dd, J=10.6, 2.6 Hz, 1H) 4.80-4.95 (m, 2H) 5.40 (s, 2H) 6.83 (d, J=8.7 Hz, 1H) 6.90-6.97 (m, 1H) 6.98-7.05 (m, 1H) 7.32-7.41 (m, 3H) 7.44-7.49 (m, 2H) 7.70-7.81 (m, 2H) 8.24 (d, J=2.3 Hz, 1H).

An analogous procedure was followed to obtain the following intermediates.

| Structure | Starting Material | Characterization |
|---|---|---|
| 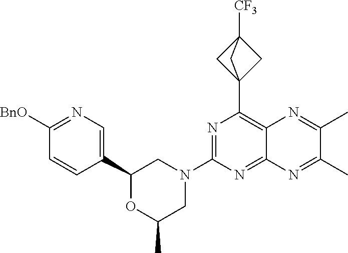 [SMILES: Cc1nc2nc(N3C[C@@H](C)O[C@@H](c4ccc(OCc5ccccc5)nc4)C3)nc(C34CC(C(F)(F)F)(C3)C4)c2nc1C] Int-C15 | 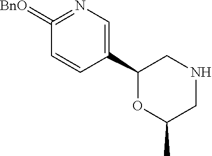 Int-C1 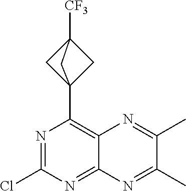 [SMILES: Cc1nc2nc(Cl)nc(C34CC(C(F)(F)F)(C3)C4)c2nc1C] | Modification: The reaction was performed starting from (2S,6R)-2-(2- benzyloxy-1,2-dihydropyridin-5-yl)- 6-methyl-morpholine (Int-C1) and 2- chloro-6,7-dimethyl-4-[3- (trifluoromethyl)-1- bicyclo[1.1.1]pentanyl]pteridine in a MW vial, using anhydrous THF as solvent, at 65 °C for 3 h. The resulting crude was purified by normal phase flash column chromatography (12 g SiO₂ column) using an elution gradient of 0-10% EtOAc in DCM to yield (2S,6R)-2-(6-benzyloxy-3- pyridyl)-4-[6,7-dimethyl-4-[3- (trifluoromethyl)-1- bicyclo[1.1.1]pentanyl]pteridin-2-yl]- 6-methyl-morpholine (Int-C15, 123 mg, 0.21 mmol, 58% yield) as a solid. ¹H NMR (CDCl₃, 400 MHz): δ 8.26 (1H, s), 7.74 (1H, d, J = 8.5 Hz), 7.28- 7.46 (5H, m), 6.86 (1H, d, J = 8.5 Hz), 5.43 (2H, s), 4.94-5.05 (1H, m), 4.57- |
| | | 4.60 (1H, m), 3.81-3.89 (1H, m), 2.93- |
| | | 3.01 (1H, m), 2.78-2.85 (2H, m), 2.69 |
| | | (3H, s), 2.64 (3H, s), 2.58 (6H, s), 1.35 |
| | | (3H, d, J = 6.0 Hz). ¹⁹F NMR (CDCl₃, |
| | | 376 MHz): δ −73.1 (3F, s). |
| 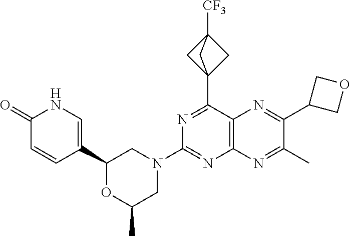 [SMILES: Cc1nc2nc(N3C[C@@H](C)O[C@@H](c4ccc(=O)[nH]c4)C3)nc(C34CC(C(F)(F)F)(C3)C4)c2nc1C1COC1] Int-C18 | 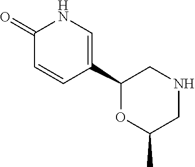 Int-C17 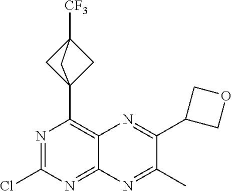 Int-C14 | Modification: The reaction was run in THF at 70 °C for 45 min. The reaction crude was purified by flash chromatography (Isco RediSep ® column 4 g, using a gradient from 0% to 2% MeOH in DCM). The selected fractions were evaporated to provide 5-[(2S,6R)-6-methyl-4-[7-methyl-6- (oxetan-3-yl)-4-[3-(trifluoromethyl)- 1-bicyclo[1.1.1]pentanyl]pteridin-2- yl]morpholin-2-yl]-1H-pyridin-2-one (Int-C18, 63% yield) as a solid. ¹H NMR (CDCl₃, 400 MHz): δ 7.64-7.69 (m, 2H), 6.73-6.76 (m, 1H), 5.09 (d, J = 7.73 Hz, 4H), 4.93-5.00 (m, 1H), 4.60-4.68 (m, 1H), 4.43-4.47 (m, 1H), 3.79-3.87 (m, 1H), 2.98 (s, 1H), 2.84- 2.92 (m, 1H), 2.75-2.82 (m, 1H), 2.66- 2.71 (m, 1H), 2.61 (s, 6H), 2.54 (s, 3H), 1.32-1.37 (m, 3H). ¹⁹F NMR (CDCl₃, 376 MHz): δ −73.0 (s, 3F). ESI-MS: [M + H]⁺ = 529.3. |
| 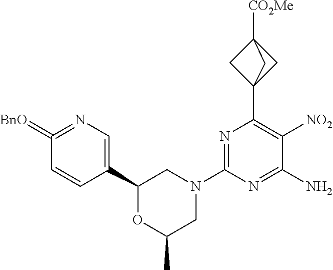 Int-C47 | 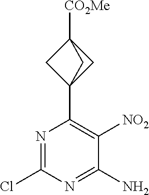 [SMILES: COC(=O)C12CC(c3nc(Cl)nc(N)c3[N+](=O)[O-])(C1)C2] Int-C46 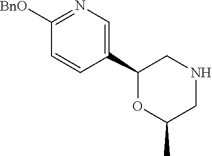 Int-C1 | Modification: Int-C1 (1 equiv.), Int- C46 (1.2 equiv.), and DIPEA (3 equiv.) were reacted in THF solvent at 70 °C for 1 h and then concentrated under reduced pressure. The residue was purified by normal phase chromatography (0-40% EtOAc in hexanes) to afford methyl 3-[6-amino- 2-[(2S,6R)-2-(6-benzyloxy-3- pyridyl)-6-methyl-morpholin-4-yl]-5- nitro-pyrimidin-4- yl]bicyclo[1.1.1]pentane-1- carboxylate (Int-C47, 464 mg, 0.85 mmol, 86% yield) as a solid. ESI-MS: [M + H]⁺ = 547.3. |
| 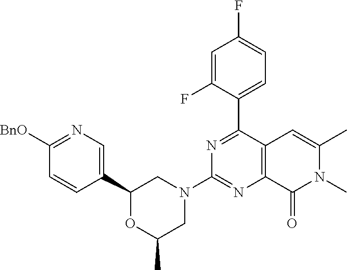 [SMILES: Cc1cc2c(-c3ccc(F)cc3F)nc(N3C[C@@H](C)O[C@@H](c4ccc(OCc5ccccc5)nc4)C3)nc2c(=O)n1C] Int-D34 | 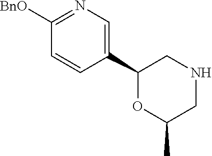 Int-C1 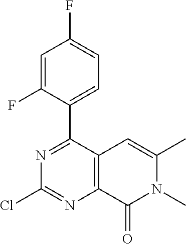 Int-D33 | Modifications: The reaction was run at 100 °C for 1 h, and then poured into water. The precipitate was collected by filtration to obtain 2-[(2S,6R)-2- (6-benzyloxy-3-pyridyl)-6-methyl- morpholin-4-yl]-4-(2,4- difluorophenyl)-6,7-dimethyl- pyrido[3,4-d]pyrimidin-8-one (Int- D34, 600 mg, 0.927 mmol, 99% yield). LCMS: [M + H]⁺ = 570.2. ¹H NMR (400 MHz, CDCl₃) δ 8.24 (d, J = 2.0 Hz, 1H), 7.72 (dd, J = 2.3, 8.6 Hz, 1H), 7.53-7.44 (m, 3H), 7.41- 7.29 (m, 4H), 7.06 (dt, J = 2.0, 8.3 Hz, 1H), 7.02-6.95 (m, 1H), 6.82 (d, J = 8.6 Hz, 1H), 6.00 (d, J = 3.1 Hz, 1H), 5.40 (s, 2H), 5.00-4.83 (m, 2H), 4.59 (dd, J = 2.4, 10.8 Hz, 1H), 3.91-3.82 (m, 1H), 3.62 (s, 3H), 2.97 (dd, J = 10.9, 13.1 Hz, 1H), 2.83 (dd, J = 10.8, 13.3 Hz, 1H), 2.34 (s, 3H), 1.35 (d, J = 6.2 Hz, 3H). |
| 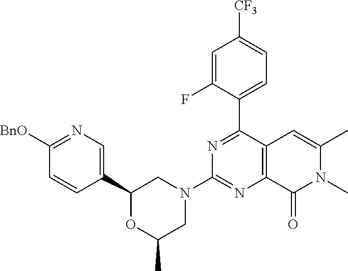 [SMILES: Cc1cc2c(-c3ccc(C(F)(F)F)cc3F)nc(N3C[C@@H](C)O[C@@H](c4ccc(OCc5ccccc5)nc4)C3)nc2c(=O)n1C] Int-D37 | 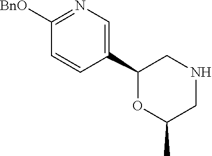 Int-C1 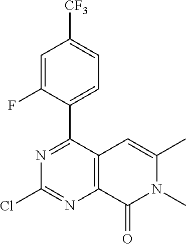 Int-D36 | Modifications: The reaction was run at 100 °C for 1 h, and then poured into water. The precipitate was collected by filtration to obtain 2-[(2S,6R)-2- (6-benzyloxy-3-pyridyl)-6-methyl- morpholin-4-yl]-4-[2-fluoro-4- (trifluoromethyl)phenyl]-6,7- dimethyl-pyrido[3,4-d]pyrimidin-8- one (Int-D37, 200 mg, 0.323 mmol, 60% yield). LCMS: [M + H]⁺ = 620.2. ¹H NMR (400 MHz, DMSO-d₆) δ 8.28-8.20 (m, 1H), 7.98-7.76 (m, 4H), 7.51-7.29 (m, 6H), 6.91 (d, J = 8.5 Hz, 1H), 6.04 (d, J = 2.5 Hz, 1H), 5.37 (s, 2H), 4.77-4.60 (m, 3H), 3.56- 3.48 (m, 3H), 3.04-2.95 (m, 2H), 2.82-2.71 (m, 1H), 2.32 (s, 3H), 1.29-1.24 (m, 3H). |
| 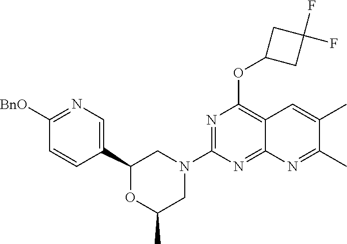 [SMILES: Cc1cc2c(OC3CC(F)(F)C3)nc(N3C[C@@H](C)O[C@@H](c4ccc(OCc5ccccc5)nc4)C3)nc2nc1C] Int-E13 | 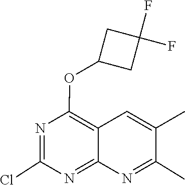 Int-E12 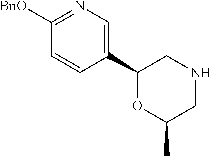 Int-C1 | Modifications: The reaction was run in THF solvent at 70 °C for 90 min and purified by flash chromatography (Isco RediSep ® column 12 g, using a gradient from 0-50% EtOAc in hexanes). The selected fractions were evaporated to provide (2S,6R)-2-(6- (benzyloxy)pyridine-3-yl)-4-(4-(3,3- difluorocyclobutoxy)-6,7- dimethylpyrido[2,3-d]pyrimidin-2-yl (Int-E13, 66 mg, 0.12 mmol, 72%) as a solid. ¹H NMR (CDCl₃, 400 MHz): δ 8.22 (d, J = 2.3 Hz, 1H), 7.91 (s, 1H), 7.71 (dd, J = 8.6, 2.4 Hz, 1H), 7.47 (d, J = 7.5 Hz, 2H), 7.36-7.40 (m, 2H), 7.30-7.34 (m, 1H), 6.84 (d, J = 8.5 Hz, 1H), 5.40 (s, 2H), 4.54 (dd, J = 10.8, 2.6Hz, 1H), 3.79-3.84 (m, 1H), 3.10- 3.21 (m, 2H), 2.89-2.96 (m, 1H), 2.76- 2.84 (m, 1H), 2.64 (s, 3H), 2.36 (s, 3H), 1.33 (d, J = 6.2 Hz, 3H). ESI- MS: [M + H]⁺ = 548.3. |

### Method Int 67. Intermediate Int-C4:5-[(2S,6R)-4-[7-(2,4-difluorophenyl)-2-(dimethylamino) thiazolo[4,5-d]pyrimidin-5-yl]-6-methyl-morpholin-2-yl]-1-(methyleneamino)pyridin-2-one

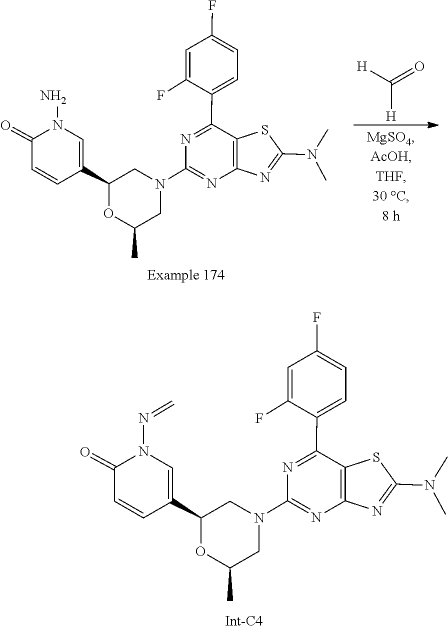 [SMILES: C[C@@H]1CN(c2nc(-c3ccc(F)cc3F)c3sc(N(C)C)nc3n2)C[C@H](c2ccc(=O)n(N)c2)O1 ; C=O ; C=Nn1cc([C@H]2CN(c3nc(-c4ccc(F)cc4F)c4sc(N(C)C)nc4n3)C[C@@H](C)O2)ccc1=O]

To a solution of 1-amino-5-[(2S,6R)-4-[7-(2,4-difluorophenyl)-2-(dimethylamino) thiazolo[4,5-d]pyrimidin-5-yl]-6-methyl-morpholin-2-yl]pyridin-2-one (Example 174, 1 equiv., 100 mg, 0.200 mmol) and formaldehyde (2 equiv., 0.030 mL, 0.400 mmol) in THF (5 mL) was added MgSO₄ (10 equiv., 240 mg, 2.00 mmol), followed by AcOH (0.25 equiv., 6.9 mg, 0.050 mmol). The mixture was stirred for 2 h at 30° C., and then added into NaHCO₃ aqueous solution (10 mL) and extracted with DCM (10 mL×2). The organics was washed with 10 mL saturated brine solution, separated, dried (Na₂SO₄) and concentrated to yield 5-[(2S,6R)-4-[7-(2,4-difluorophenyl)-2-(dimethylamino) thiazolo[4,5-d]pyrimidin-5-yl]-6-methyl-morpholin-2-yl]-1-(methyleneamino)pyridin-2-one (Int-C4, 60 mg, 0.117 mmol, 59% yield), which was used for next step directly. LCMS: [M+H]⁺=512.1; purity=51% (UV 220 nm); Ret. time=0.569 min.

### Method Int 68. Intermediate Int-C5: tert-butyl ((diphenylphosphoryl)oxy)(methyl) carbamate

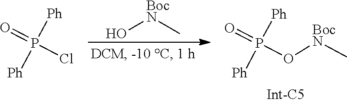

To a flame-dried round-bottomed flask under N₂ atmosphere was added tert-butyl N-hydroxy-N-methyl-carbamate (1.1 equiv., 0.68 g, 4.63 mmol) and DCM (9 mL). The mixture was cooled down to-10° C. and triethylamine (1.25 equiv., 0.73 mL, 5.27 mmol) was added dropwise over 10 min. To a separate flask diphenylphosphinic chloride (1 equiv., 1.00 g, 4.23 mmol) and DCM (6 mL) were added under N₂ atmosphere. This solution was added dropwise to the hydroxylamine solution over 30 min, such that the internal temperature of the reaction solution was kept below 0° C. After 1 h, the mixture was allowed to warm above 10° C. and an aqueous solution of citric acid (5% wt., 2.5 mL) was added over 5 min. The phases were separated and the organic layer was concentrated under reduced pressure until the product began to precipitate. Heptanes (30 mL) was added to the resulting slurry and the solvent was removed under reduced pressure. The desired product tert-butyl N-diphenylphosphoryloxy-N-methyl-carbamate (Int-C5, 1.1 g, 3.2 mmol, 75% yield) was obtained as a solid. ¹H NMR (400 MHz, CDCl₃): δ 8.01-7.90 (4H, m), 7.57-7.50 (2H, m), 7.48-7.40 (4H, m), 1.39 (9H, s).

### Method Int 69. Intermediate Int-C6: N-diphenylphosphoryloxymethanamine

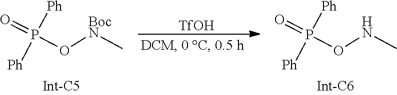

A round-bottom flask was charged with tert-butyl N-diphenylphosphoryloxy-N-methyl-carbamate (Int-C5, 1 equiv., 1.50 g, 4.32 mmol) and dissolved in DCM (80 mL) under N₂ atmosphere. The solution was cooled to 0° C. under stirring and triflic acid (2.5 equiv., 0.95 mL, 10.8 mmol) was added dropwise. The reaction warmed up to room temperature and stirred for 30 min. The reaction mixture was diluted with DCM and washed with a sat. NaHCO₃(aq) solution (50 mL) and water. The organic phase was dried over anhydrous Na₂SO₄, filtered, and concentrated under reduced pressure to yield N-diphenylphosphoryloxymethanamine (Int-C6, 0.75 g, 3.01 mmol, 70% yield) as a solid. ¹H NMR (400 MHz, CDCl₃): δ 7.91-7.79 (4H, m), 7.57-7.50 (2H, m), 7.49-7.37 (4H, m), 2.89 (3H, s).

An analogous procedure was followed to obtain the following intermediates.

| Structure | Starting Material | Characterization |
|---|---|---|
| 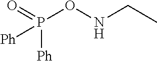 [SMILES: CCNOP(=O)(c1ccccc1)c1ccccc1] Int-C9 | 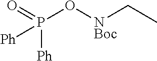 [SMILES: CCN(OP(=O)(c1ccccc1)c1ccccc1)C(=O)OC(C)(C)C] Int-C8 | Int-C8 (240 mg, 0.664 mmol) was treated with triflic acid in an analogous manner to yield N- diphenylphosphoryloxyethanamine (Int-C9, 156 mg, 0.60 mmol, 90% yield) as a solid. |

### Method Int 70. Intermediate Int-C7:2-chloro-6-(difluoromethyl)-7-methyl-4-[3-(trifluoromethyl)-1-bicyclo[1.1.1]pentanyl]pteridine

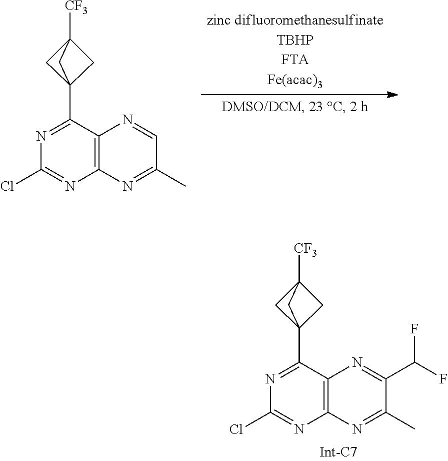 [SMILES: Cc1cnc2c(C34CC(C(F)(F)F)(C3)C4)nc(Cl)nc2n1 ; Cc1nc2nc(Cl)nc(C34CC(C(F)(F)F)(C3)C4)c2nc1C(F)F]

To a solution of 2-chloro-7-methyl-4-[3-(trifluoromethyl)-1-bicyclo[1.1.1]pentanyl]pteridine (1 equiv., 500 mg, 1.59 mmol) and TFA (2 equiv., 243 μL, 3.18 mmol) in a 1:1 mixture of DMSO (30 mL) and DCM (30 mL) was added zinc difluoromethanesulfinate (3 equiv., 1.40 g, 4.77 mmol), followed by tert-butyl hydroperoxide solution (70 wt. % in H₂O, 5 equiv., 1.1 mL, 7.94 mmol) and iron (III) acetylacetonate (0.1 equiv., 56 mg, 0.159 mmol) under vigorous stirring. The reaction mixture was stirred at room temperature until full conversion was observed by LC-MS. The reaction mixture was partitioned between DCM (10 mL) and sat. NaHCO₃(aq)(10 mL). The layers were separated, and the aqueous layer was extracted with DCM (2× 10 mL). The combined organic layers were dried over anhydrous Na₂SO₄, filtered and concentrated under reduced pressure. The resulting crude material was purified by flash column chromatography (40 g SiO₂ column) using an elution gradient of 0-100% EtOAc in hexanes to yield (2-chloro-6-(difluoromethyl)-7-methyl-4-[3-(trifluoromethyl)-1-bicyclo[1.1.1]pentanyl]pteridine (Int-C7, 204 mg, 0.425 mmol, 27% yield) as a solid. ¹H NMR (CDCl₃, 400 MHz): δ 6.83 (1H, t, J=53.8 Hz), 3.03 (3H, s), 2.68 (6H, s). ¹⁹F NMR (CDCl₃, 376 MHz): δ −73.1 (3F, s), -115.8 (2F, d, J=54.0 Hz).

An analogous procedure was followed to obtain the following intermediates.

| Structure | Starting Material | Characterization |
|---|---|---|
| 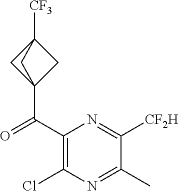 [SMILES: Cc1nc(Cl)c(C(=O)C23CC(C(F)(F)F)(C2)C3)nc1C(F)F] Int-D16 | 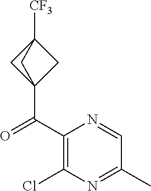 Int-C20 | Modification: The crude material was purified by flash chromatography (Isco RediSep ® column 40 g, using a gradient from 20-60% EtOAc in hexanes). The selected fractions were evaporated to yield (3-chloro-6- (difluoromethyl)-5-methylpyrazin-2-yl)(3- (trifluoromethyl)bicyclo[1.1.1]pentan-1-yl)methanone (Int-D16, 162 mg, 0.48 mmol, 28% yield) as a solid. ESI- MS: [M + H]⁺ = 338.9. ¹H NMR (400 MHz, DMSO-d₆) δ 7.36 (t, J = 52.9 Hz, 1H), 2.45 (s, 3H). ¹⁹F NMR (376 MHz, DMSO-d₆) δ −71.5 (s, 3F), −118.2 (dd, J = 53.1, 1.6 Hz, 2F) |

### Method Int 71. Intermediate Int-C8: tert-butyl N-diphenylphosphoryloxy-N-ethyl-carbamate

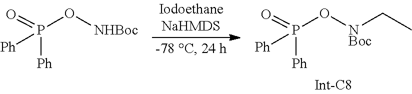 [SMILES: CC(C)(C)OC(=O)NOP(=O)(c1ccccc1)c1ccccc1 ; CCN(OP(=O)(c1ccccc1)c1ccccc1)C(=O)OC(C)(C)C]

To a 50 mL round-bottom flask under N₂ atmosphere was added tert-butyl N-diphenylphosphoryloxycarbamate (1 equiv., 0.95 g, 2.84 mmol) and anhydrous THF (15 mL). The reaction mixture was cooled down to-78° C., NaHMDS (1.0 M in THF, 1.5 equiv., 4.3 mL, 4.25 mmol) was added dropwise, and the mixture was stirred at −78° C. for 30 min. After 30 min, a solution of iodoethane (1.7 equiv., 0.39 mL, 4.82 mmol) in anhydrous THF (4.5 mL) was added dropwise to the reaction mixture. The reaction mixture was allowed to slowly warm up to r.t. and stirred for 24 h. The reaction was quenched by addition of sat. NH₄Cl (aq.)(10 mL) and extracted with EtOAc (3×40 mL). The combined organic phases were washed with brine, dried over anhydrous Na₂SO₄, filtered, and concentrated under reduced pressure. The crude material was purified by normal phase flash chromatography (40 g SiO₂ column) using an elution gradient of 5-60% EtOAc in hexanes to yield tert-butyl N-diphenylphosphoryloxy-N-ethyl-carbamate (Int-C8, 0.28 g, 0.78 mmol, 27% yield) as an oil.

### Method Int 72. Intermediate Int-C11:1-cyclopropyl-5-((2S,6R)-6-methyl-4-((4-nitrophenyl) sulfonyl) morpholin-2-yl)pyridine-2 (1H)-one

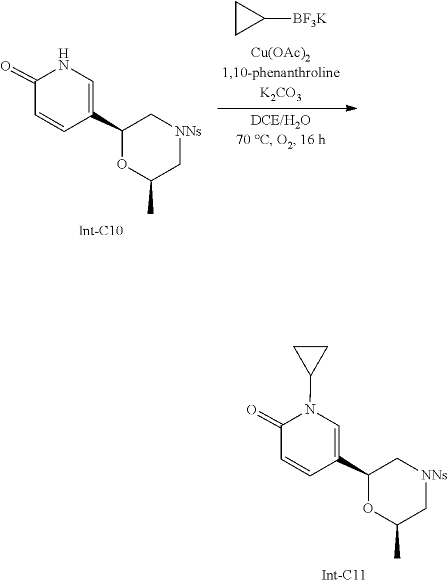

To a microwave vial equipped with Teflon-coated magnetic stirring bar was added potassium carbonate (2 equiv., 364 mg, 2.64 mmol), 1,10-phenanthroline (0.13 equiv., 29.7 mg, 0.17 mmol), 5-[(2S,6R)-6-methyl-4-(4-nitrophenyl) sulfonyl-morpholin-2-yl]-1H-pyridin-2-one 2 (Int-C9, 1 equiv., 500 mg, 1.32 mmol), Cu(OAc)₂ (0.25 equiv., 59.8 mg, 0.33 mmol) and potassium cyclopropyltrifluoroborate (3 equiv., 585 mg, 3.95 mmol), DCE (5 mL) and water (1.7 mL). O₂ gas was bubbled into the reaction mixture for 5 min, and the reaction mixture was stirred at 70° C. overnight under an oxygen atmosphere. After cooling to room temperature, the reaction mixture was concentrated under reduced pressure and purified by flash chromatography (24 g SiO₂ column) using an elution gradient of 60-100% EtOAc in hexanes to yield 1-cyclopropyl-5-((2S,6R)-6-methyl-4-((4-nitrophenyl) sulfonyl) morpholin-2-yl)pyridine-2 (1H)-one (Int-C11, 377 mg, 0.90 mmol, 68% yield) as a solid. ESI-MS: [M+H]⁺=420.2. ¹H NMR (CDCl₃, 400 MHz): δ 8.42-8.39 (m, 2H), 7.95-7.92 (m, 2H), 7.31 (d, J=2.5 Hz, 1H), 7.23-7.25 (m, 1H), 6.61 (d, J=9.4 Hz, 1H), 4.40-4.43 (m, 1H), 3.83-3.89 (m, 1H), 3.69-3.75 (m, 2H), 3.34-3.26 (m, 1H), 2.18-2.07 (m, 2H), 1.23 (d, J=6.2 Hz, 3H), 1.11-1.17 (m, 2H), 0.83-0.87 (m, 2H).

An analogous procedure was followed to obtain the following intermediates.

| Structure | Starting Material | Characterization |
|---|---|---|
| 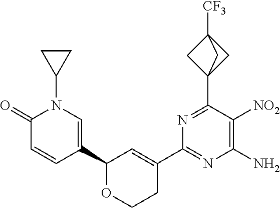 [SMILES: Nc1nc(C2=C[C@H](c3ccc(=O)n(C4CC4)c3)OCC2)nc(C23CC(C(F)(F)F)(C2)C3)c1[N+](=O)[O-]] Int-C35 | 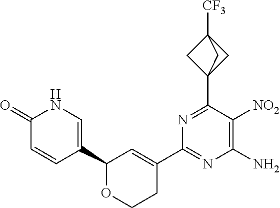 [SMILES: Nc1nc(C2=C[C@H](c3ccc(=O)[nH]c3)OCC2)nc(C23CC(C(F)(F)F)(C2)C3)c1[N+](=O)[O-]] | (R)-5-(4-(4-amino-5-nitro-6-(3- (trifluoromethyl)bicyclo[1.1.1]pentan-1- yl)pyrimidin-2-yl)-5,6-dihydro-2H- pyran-2-yl)pyridin-2(1H)-one was N- alkylated via the above procedure and purified by silica gel flash column chromatography using an elution gradient of 20-60% EtOAc in hexanes to yield (R)-5-(4-(4-amino-5-nitro-6-(3- (trifluoromethyl)bicyclo[1.1.1]pentan-1- yl)pyrimidin-2-yl)-5,6-dihydro-2H- pyran-2-yl)-1-cyclopropylpyridin- 2(1H)-one (Int-C35, 313 mg, 0.64 mmol, 82% yield) as a solid. ESI-MS: [M + H]⁺ = |
| | | 490.3. ¹H NMR (CDCl₃, 400 MHz): δ |
| | | 7.31 (2H, m), 7.18 (1H, s), 6.71 (1H, br |
| | | s), 6.54 (1H, d, J = 9.3 Hz), 5.08 (1H, s), |
| | | 4.10 (1H, dt, J = 11.4, 4.1 Hz), 3.75-3.81 |
| | | (1H, m), 3.30 (1H, s), 2.63 (2H, s), 2.39 |
| | | (6H, s), 1.11 (2H, d, J = 6.6 Hz), 0.85 |
| | | (2H, s). |

### Method Int 73. Intermediate Int-C14:2-chloro-7-methyl-6-(oxetan-3-yl)-4-[3-(trifluoromethyl)-1-bicyclo[1.1.1]pentanyl]pteridine

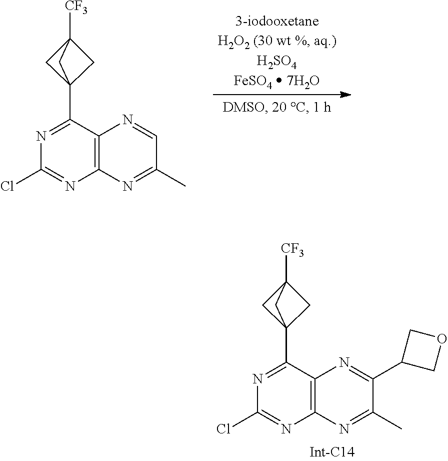 [SMILES: Cc1cnc2c(C34CC(C(F)(F)F)(C3)C4)nc(Cl)nc2n1 ; Cc1nc2nc(Cl)nc(C34CC(C(F)(F)F)(C3)C4)c2nc1C1COC1]

Hydrogen peroxide (30% wt. in water, 3 equiv., 974 μL, 9.53 mmol) was added dropwise over 2 min to a stirring solution of 2-chloro-7-methyl-4-[3-(trifluoromethyl)-1-bicyclo[1.1.1]pentanyl]pteridine (1 equiv., 1.0 g, 3.2 mmol), concentrated sulfuric acid (2 equiv., 339 μL, 6.4 mmol), 3-iodooxetane (2 equiv., 559 μL, 6.4 mmol), and iron (II) sulfateheptahydrate (0.3 equiv., 267 mg, 0.95 mmol) in DMSO (20 mL) at room temperature. After 1-2 min another portion of iron (II) sulfateheptahydrate (0.3 equiv., 267 mg, 0.95 mmol) was added and the mixture was stirred at room temperature for 60 min. The mixture was poured into a mixture of sat. NaHCO₃ (aq.)(10 mL) and EtOAc (15 mL). The aqueous and organic layers were partitioned, and the aqueous layer was extracted with EtOAc (2×15 mL). The combined organic extracts were washed with brine, dried over anhydrous Na₂SO₄, filtered, and concentrated under reduced pressure. The resulting oil was purified by normal phase flash chromatography (40 g SiO₂ column) using an elution gradient of 1-100% EtOAc in hexanes to yield 2-chloro-7-methyl-6-(oxetan-3-yl)-4-[3-(trifluoromethyl)-1-bicyclo[1.1.1]pentanyl]pteridine (Int-C14, 270 mg, 0.73 mmol, 23% yield) as a solid. ESI-MS: [M+H]⁺=371.3.

### Method Int 74. Intermediate Int-C17:5-((2S,6R)-6-methylmorpholin-2-yl)pyridin-2 (1H)-one

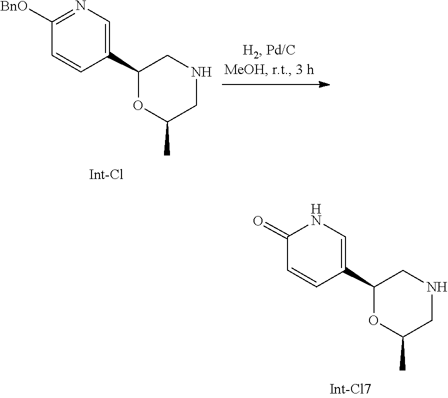 [SMILES: C[C@@H]1CNC[C@H](c2ccc(OCc3ccccc3)nc2)O1 ; C[C@@H]1CNC[C@H](c2ccc(=O)[nH]c2)O1]

To a flame-dried round-bottom flask equipped with a Teflon-coated magnetic stirring bar were added (2S,6R)-2-(6-benzyloxy-3-pyridyl)-6-methyl-morpholine (Int-C1, 1 equiv., 200 mg, 0.7 mmol) and MeOH (13 mL). The reaction was purged using vacuum/nitrogen cycles three times and 10 wt % Pd/C (0.27 equiv., 20.0 mg, 0.19 mmol) was added. The reaction vial was cycled with H₂ (g) and vacuum three times, equipped with a H₂ (g) balloon, and stirred under 1 atm of H₂ (g) for 30 min. The reaction mixture was then filtered through a pad of Celite, washed with DCM, and concentrated under reduced pressure to afford 5-((2S,6R)-6-methylmorpholin-2-yl)pyridin-2 (1H)-one (Int-C17, 124 mg, 0.64 mmol, 91% yield) as an oil. ¹H NMR (CD₃OD, 400 MHz): δ 7.61 (dd, J=9.40, 2.6 Hz, 1H), 7.40 (d, J=2.5 Hz, 1H), 6.53 (d, J=9.4 Hz, 1H), 4.33-4.36 (m, 1H), 3.73 (dd, J=10.4, 6.1 Hz, 1H), 3.34 (s, 1H), 2.95 (d, J=12.8 Hz, 1H), 2.88 (d, J=12.6 Hz, 1H), 2.56 (dd, J=12.7, 10.7 Hz, 1H), 2.44 (dd, J=12.7, 10.5 Hz, 1H), 1.16 (d, J=6.2 Hz, 3H).

An analogous procedure was followed to obtain the following intermediates.

| Structure | Starting Material | Characterization |
|---|---|---|
| 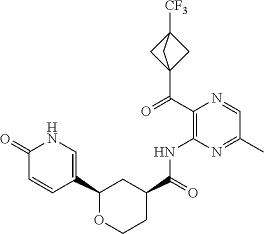 [SMILES: Cc1cnc(C(=O)C23CC(C(F)(F)F)(C2)C3)c(NC(=O)[C@H]2CCO[C@@H](c3ccc(=O)[nH]c3)C2)n1] Int-C24 | 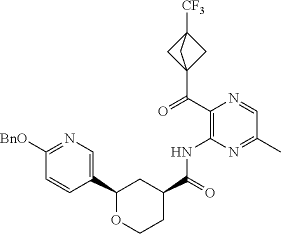 Int-C23 | Modification: The reaction mixture was stirried for 30 min under an atmosphere of hydrogen, then purged with argon and filtered through Celite, washing with a 4:1 solution of DCM/MeOH. The filtrate was concentrated under vacuum to obtain (2R,4S)-N-(6-methyl-3-(3- (trifluoromethyl)bicyclo[1.1.1]pentane- 1-carbonyl)pyrazin-2-yl)-2-(6-oxo- 1,6-dihydropyridin-3-yl)tetrahydro- 2H-pyran-4-carboxamide (Int-C24, 0.97 g, 1.91 mmol, 95% yield) as a foam that was used without further purification. ESI-MS: [M + H]⁺ = 477.3. ¹H NMR (CDCl₃, 400 MHz): δ |
| | | 11.22 (1H, s), 8.23 (1H, s), 7.59 (1H, |
| | | dd, J = 9.4, 2.5 Hz), 7.48 (1H, d, J = |
| | | 2.4 Hz), 6.67 (1H, d, J = 9.4 Hz), 4.24 |
| | | (2H, d, J = 11.6 Hz), 3.61-3.68 (1H, |
| | | m), 2.94-3.00 (1H, m), 2.64 (3H, s), |
| | | 2.51 (6H, s), 2.13 (1H, d, J = 13.3 Hz), |
| | | 1.93-1.99 (2H, m), 1.80 (1H, dd, J = |
| | | 24.5, 12.2 Hz). |
| 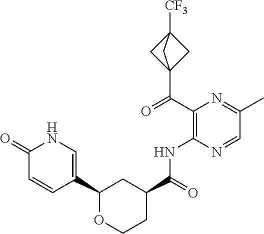 [SMILES: Cc1cnc(NC(=O)[C@H]2CCO[C@@H](c3ccc(=O)[nH]c3)C2)c(C(=O)C23CC(C(F)(F)F)(C2)C3)n1] Int-C34 | 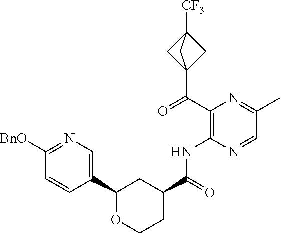 Int-C33 | Modification: The reaction mixture was stirried for 30 min under an atmosphere of hydrogen, then purged with argon and filtered through Celite, washing with a 4:1 solution of DCM/MeOH. The filtrate was concentrated under vacuum to obtain (2R,4S)-N-(5-methyl-3-(3- (trifluoromethyl)bicyclo[1.1.1]pentane- 1-carbonyl)pyrazin-2-yl)-2-(6-oxo- 1,6-dihydropyridin-3-yl)tetrahydro- 2H-pyran-4-carboxamide (Int-C34, 1.31 g, 2.48 mmol, 93% yield) as a foam that was used without further purification. ESI-MS: [M + H]⁺ = 477.4. ¹H NMR (CDCl₃, 400 MHz): δ 11.08 |
| | | (1H, s), 8.51 (1H, s), 7.63 (1H, dd, J = |
| | | 9.4, 2.5 Hz), 7.55 (1H, d, J = 2.4 Hz), |
| | | 6.71 (1H, d, J = 9.4 Hz), 4.22-4.27 (2H, |
| | | m), 3.66 (1H, dd, J = 13.3, 10.3 Hz), |
| | | 2.84-2.92 (1H, m), 2.59 (3H, s), 2.53 |
| | | (6H, s), 2.14 (1H, d, J = 13.5 Hz), 1.92- |
| | | 2.01 (2H, m), 1.80 (1H, dd, J = 24.5, |
| | | 12.2 Hz). |
| 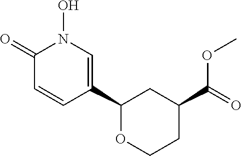 [SMILES: COC(=O)[C@H]1CCO[C@@H](c2ccc(=O)n(O)c2)C1] Int-C42 | 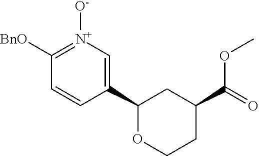 Int-C41 | Modification: The reaction was run with 0.1 equiv. Pd/C (10 wt %) in EtOH for 1 h to obtain methyl (2R,4S)-2-(1- hydroxy-6-oxo-1,6-dihydropyridin-3- yl)tetrahydro-2H-pyran-4-carboxylate (Int-C42, 303 mg, 1.2 mmol, 99% yield) as a gum. ESI-MS: [M + H]⁺ = 254.2. |
| 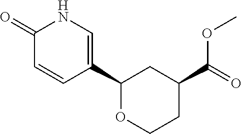 [SMILES: COC(=O)[C@H]1CCO[C@@H](c2ccc(=O)[nH]c2)C1] Int-D2 | 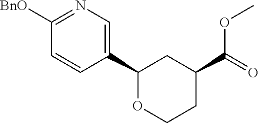 [SMILES: COC(=O)[C@H]1CCO[C@@H](c2ccc(OCc3ccccc3)nc2)C1] Int-D1 | Modification: The reaction was run with 0.03 equiv. Pd/C (10 wt%) in EtOH for 16 h to obtain methyl (2R,4S)-2-(6-oxo-1H-pyridin-3- yl)tetrahydropyran-4-carboxylate (Int- D2, 6.6 g, 27.9 mmol, 97% yield) as a solid. ¹H NMR (CDCl₃, 400 MHz): δ 12.00 (br s, 1H), 7.48 (dd, J = 9.5, 2.4 Hz, 1H), 7.32 (d, J = 2.1 Hz, 1H), 6.57 (d, J = 9.4 Hz, 1H), 4.18-4.12 (m, 2H), |
| | | 3.70 (s, 3H), 3.57 (td, J = 12.0, 2.2 Hz, |
| | | 1H), 2.68 (tt, J = 12.2, 3.9 Hz, 1H), 2.06 |
| | | (d, J = 13.5 Hz, 1H), 1.90 (dd, J = 13.5, |
| | | 1.8 Hz, 1H), 1.77 (ddd, J = 25.5, 12.5, |
| | | 4.5 Hz, 1H), 1.64 (q, J = 12.4 Hz, 1H). |
| | | ESI-MS: [M + H]⁺ = 238.2. |
| 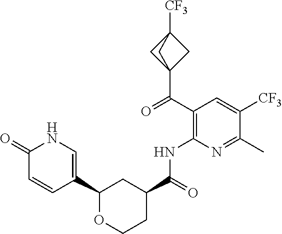 [SMILES: Cc1nc(NC(=O)[C@H]2CCO[C@@H](c3ccc(=O)[nH]c3)C2)c(C(=O)C23CC(C(F)(F)F)(C2)C3)cc1C(F)(F)F] Int-E11 | 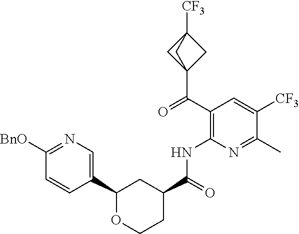 Int-E8 | Modifications: The reaction was run in 1:1 EtOH/EtOAc as solvent to afford (2R,4S)-N-(6-methyl-5- (trifluoromethyl)-3-(3- (trifluoromethyl)bicyclo- [1.1.1]pentane-1-carbonyl)pyridin-2- yl)-2-(6-oxo-1,6-dihydropyridin-3- yl)tetrahydro-2H-pyran-4-carboxamide (Int-E11, 0.45 g, 0.84 mmol, 99% yield) as a solid that was used without further purification. ESI-MS: [M + H]⁺ = 546.3. ¹H NMR (400 MHz, CDCl₃): δ 11.03 (s, 1H), 8.39 (s, 1H), 7.61 (dd, J = 9.4, 2.3 Hz, 1H), 7.51 (s, 1H), 6.69 (d, J = 9.4 Hz, 1H), 4.26-4.21 (m, 2H), 3.65 (td, J = 11.6, 3.3 Hz, 1H), 3.13-3.07 (m, |
| | | 1H), 2.72-2.69 (m, 3H), 2.49 (s, 6H), |
| | | 2.28-2.41 (0H), 2.11 (d, J = 14.1 Hz, |
| | | 1H), 1.98-1.87 (m, 2H), 1.78 (dd, J = |
| | | 24.8, 11.9 Hz, 1H). ¹⁹F NMR (376 MHz, |
| | | CDCl₃): δ −61.8 (s, 3F), −73.2 (s, |
| | | 3F). |

### Method Int 75. Intermediate Int-C19: (2S,6R)-2-(6-(benzyloxy)pyridin-3-yl)-4-(5-(2,4-difluorophenyl)-2,3-dimethylpyrido[3,4-b]pyrazin-7-yl)-6-methylmorpholine

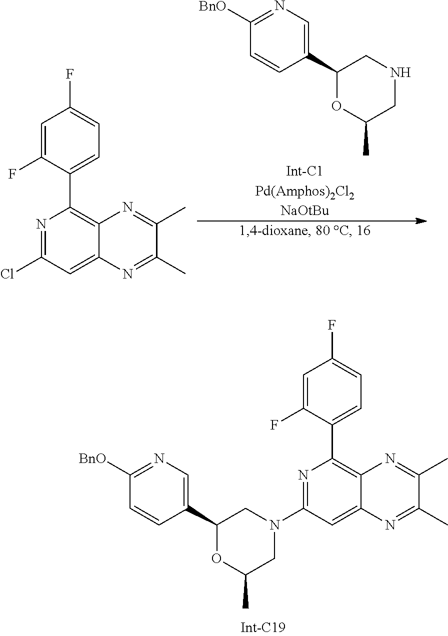 [SMILES: Cc1nc2cc(Cl)nc(-c3ccc(F)cc3F)c2nc1C ; C[C@@H]1CNC[C@H](c2ccc(OCc3ccccc3)nc2)O1 ; Cc1nc2cc(N3C[C@@H](C)O[C@@H](c4ccc(OCc5ccccc5)nc4)C3)nc(-c3ccc(F)cc3F)c2nc1C]

A mixture of 7-chloro-5-(2,4-difluorophenyl)-2,3-dimethylpyrido[3,4-b]pyrazine (1 equiv., 100 mg, 0.33 mmol), (2S,6R)-2-(6-(benzyloxy)pyridin-3-yl)-6-methylmorpholine (Int-C1, 1.1 equiv., 102 mg, 0.360 mmol), sodium tert-butoxide (4 equiv., 126 mg, 1.31 mmol) and Pd(Amphos)₂Cl₂ (0.1 equiv., 23 mg, 0.03 mmol) in a 10 mL microwave vial was subjected to three cycles of vacuum/nitrogen fill. 1,4-Dioxane (5.0 mL) was added and the mixture was stirred at 80° C. for 16 h. The mixture was cooled down to room temperature, filtered through a pad of Celite, rinsed with 10 mL of EtOAc and concentrated under reduced pressure. The crude material was purified by flash chromatography (Isco Redisep 4 g column, using a gradient from 100% hexanes to 100% EtOAc) to yield (2S,6R)-2-(6-(benzyloxy)pyridin-3-yl)-4-(5-(2,4-difluorophenyl)-2,3-dimethylpyrido[3,4-b]pyrazin-7-yl)-6-methylmorpholine (Int-C19, 76 mg, 0.137 mmol, 42% yield). ESI-MS: [M+H]⁺=554.3.

### Method Int 76. Intermediate Int-C21: (3-((2,4-dimethoxybenzyl)amino)-5-methylpyrazin-2-yl)(3-(trifluoromethyl) bicyclo[1.1.1]pentan-1-yl) methanone

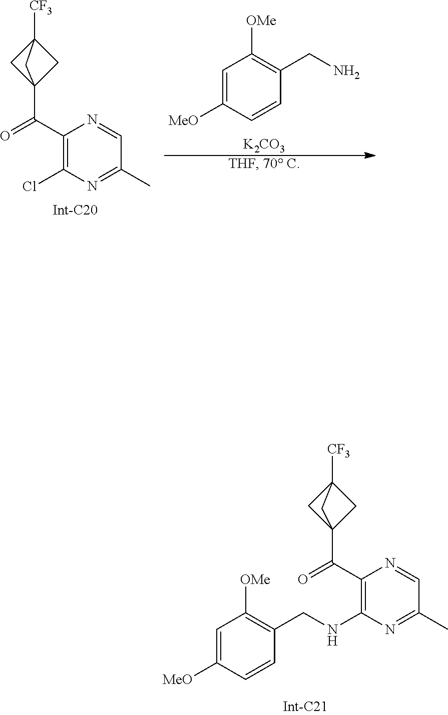

To a stirring solution of (3-chloro-5-methylpyrazin-2-yl)(3-(trifluoromethyl) bicyclo[1.1.1]pentan-1-yl) methanone (Int-C20, 1 equiv., 1.26 g, 4.33 mmol) in anhydrous THF (40 mL) was added potassium carbonate (2 equiv., 1.2 g, 8.6 mmol) and (2,4-dimethoxyphenyl) methanamine (2.1 equiv., 1.4 mL, 9.1 mmol). The reaction flask was fitted with a reflux condenser and stirred at 60° C. for 16 h. The reaction mixture was cooled to r.t. and diluted with EtOAc (150 mL). The resulting suspension was washed with sat. ammonium chloride (aq) and brine. The organic layer was dried over anhydrous Na₂SO₄, filtered, and concentrated under reduced pressure onto Celite (15 g). The crude mixture was purified by silica gel flash-column chromatography using an elution gradient of 2%-10% EtOAc in hexanes to yield (3-((2,4-dimethoxybenzyl)amino)-5-methylpyrazin-2-yl)(3-(trifluoromethyl) bicyclo[1.1.1]pentan-1-yl) methanone (Int-C21, 1.52 g, 3.6 mmol, 83% yield) as a solid. ESI-MS: [M+H]⁺=422.3. ¹H NMR (CDCl₃, 400 MHz): δ 8.99 (1H, t, J=5.7 Hz), 7.68 (1H, s), 7.24-7.26 (1H, m), 6.47 (1H, d, J=2.4 Hz), 6.41 (1H, dd, J=8.3, 2.4 Hz), 4.69 (2H, d, J=5.0 Hz), 3.87 (3H, s), 3.79 (3H, s), 2.46 (3H, s), 2.43 (6H, s).

An analogous procedure was followed to obtain the following intermediates.

| Structure | Starting Material | Characterization |
|---|---|---|
| 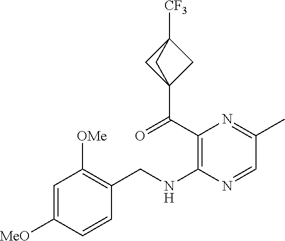 [SMILES: COc1ccc(CNc2ncc(C)nc2C(=O)C23CC(C(F)(F)F)(C2)C3)c(OC)c1] Int-C31 | 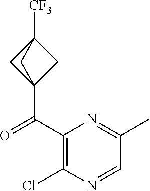 Int-C30 | The procedure yielded (3-((2,4- dimethoxybenzyl)amino)-6-methylpyrazin-2- yl)(3-(trifluoromethyl)-bicyclo[1.1.1]pentan-1- yl)methanone (Int-C31, 3.16 g, 7.50 mmol, 86% yield) as a solid. ESI-MS: [M + H]⁺ = 422.3. ¹H NMR (CDCl₃, 400 MHz): δ 8.83 (1H, t, J = 5.7 Hz), 8.13 (1H, s), 6.47 (1H, d, J = 2.4 Hz), 6.40 (1H, dd, J = 8.3, 2.4 Hz), 4.66 (2H, d, J = 5.1 Hz), 3.86 (3H, s), 3.78 (3H, s), 2.45 (6H, s), 2.41 (3H, s). |
| 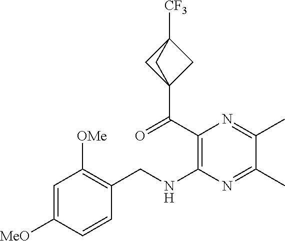 [SMILES: COc1ccc(CNc2nc(C)c(C)nc2C(=O)C23CC(C(F)(F)F)(C2)C3)c(OC)c1] Int-F2 | 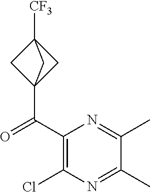 [SMILES: Cc1nc(Cl)c(C(=O)C23CC(C(F)(F)F)(C2)C3)nc1C] Int-F1 | The procedure yielded 3-((3,4- dimethylbenzyl)amino)-5,6-dimethylpyrazin-2- yl)(3-(trifluoromethyl)bicyclo[1.1.1]pentan-1- yl)methanone (Int-F2, 9.9 g, 22.8 mmol, 77% yield) as a solid. ¹H NMR (CDCl₃, 400 MHz): δ 8.75 (1H, s), 7.21 (1H, d, J = 8.3 Hz), 6.45 (1H, d, J = 2.4 Hz), 6.39 (1H, dd, J = 8.2, 2.4 Hz), 4.64 (2H, d, J = 5.9 Hz), 3.85 (3H, s), 3.77 (3H, s), 2.43 (3H, s), 2.42 (6H, s), 2.37 (3H, s). |

### Method Int 77. Intermediate Int-C22: (3-amino-5-methylpyrazin-2-yl)(3-(trifluoromethyl) bicyclo[1.1.1]pentan-1-yl) methanone

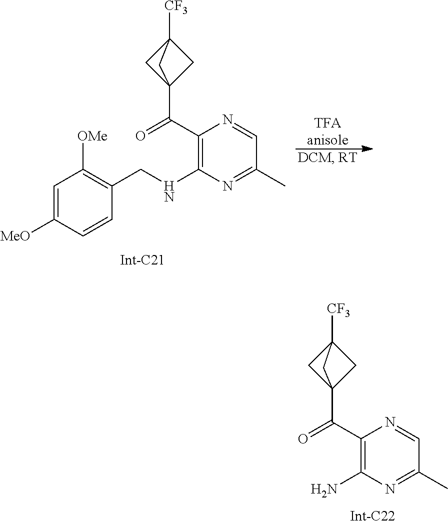

To a stirring solution of (3-((2,4-dimethoxybenzyl)amino)-5-methylpyrazin-2-yl)(3-(trifluoromethyl) bicyclo[1.1.1]pentan-1-yl) methanone (Int-C21, 1 equiv., 1.51 g, 3.58 mmol) and anisole (1 equiv., 0.39 mL, 3.58 mmol) in anhydrous DCM (50 mL) was added TFA (9.11 equiv., 2.5 mL, 32.6 mmol). The reaction mixture was stirred at r.t. for 16 h. The reaction mixture was concentrated in vacuo and diluted with EtOAc (150 mL). The resulting solution was washed with sat. NaHCO₃(aq) and brine. The organic layer was dried over anhydrous Na₂SO₄, filtered, and concentrated in vacuo onto Celite (20 g). The crude mixture was purified by silica gel flash-column chromatography using an elution gradient of 10%- 25% EtOAc in hexanes to yield (3-amino-5-methylpyrazin-2-yl)(3-(trifluoromethyl) bicyclo[1.1.1]pentan-1-yl) methanone (Int-C22, 0.93 g, 3.44 mmol, 96% yield) as a solid. ESI-MS: [M+H]⁺=272.2. ¹H NMR (400 MHz, CDCl₃): δ 7.84 (1H, s), 2.47 (6H, s), 2.44 (3H, s).

An analogous procedure was followed to obtain the following intermediates.

| Structure | Starting Material | Characterization |
|---|---|---|
| 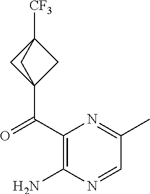 [SMILES: Cc1cnc(N)c(C(=O)C23CC(C(F)(F)F)(C2)C3)n1] Int-C32 | 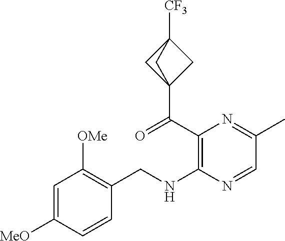 Int-C31 | The procedure yielded (3-amino-6- methylpyrazin-2-yl)(3- (trifluoromethyl)bicyclo[1.1.1]pentan-1- yl)methanone (Int-C32, 1.81 g, 6.68 mmol, 89% yield) as a crystalline solid. ESI-MS: [M + H]⁺ = 272.2. ¹H NMR (400 MHz, CDCl₃): δ 8.09 (1H, s), 2.48 (6H, s), 2.45 (3H, s). |
| 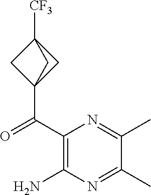 [SMILES: Cc1nc(N)c(C(=O)C23CC(C(F)(F)F)(C2)C3)nc1C] Int-F3 | 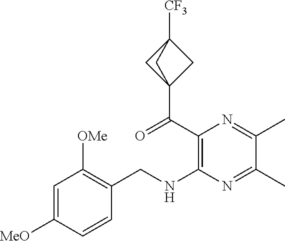 [SMILES: COc1ccc(CNc2nc(C)c(C)nc2C(=O)C23CC(C(F)(F)F)(C2)C3)c(OC)c1] Int-F2 | The procedure afforded (3-amino-5,6-dimethyl- pyrazin-2-yl)-[3-(trifluoromethyl)-1- bicyclo[1.1.1]pentanyl]methanone (Int-F3, 6.0 g, 21.0 mmol, 92% yield) as a solid. ¹H NMR (CDCl₃, 400 MHz): δ 2.47 (6H, s), 2.43 (3H, s), 2.42 (3H, s). |

### Method Int 78. Intermediate Int-C23: (2R,4S)-2-(6-(benzyloxy)pyridin-3-yl)-N-(6-methyl-3-(3-(trifluoromethyl) bicyclo[1.1.1]pentane-1-carbonyl) pyrazin-2-yl)tetrahydro-2H-pyran-4-carboxamide

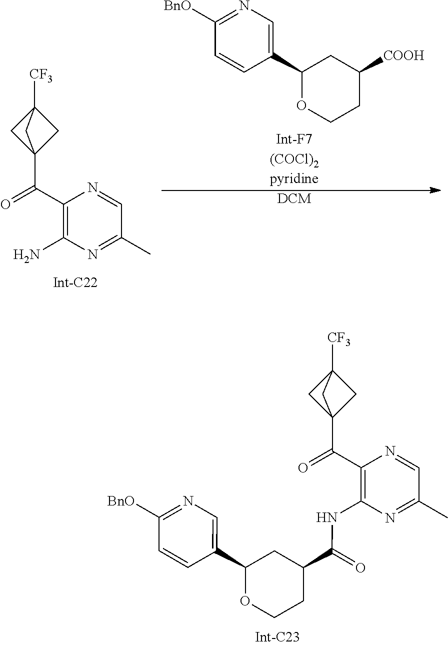 [SMILES: Cc1cnc(C(=O)C23CC(C(F)(F)F)(C2)C3)c(N)n1 ; O=C(O)[C@H]1CCO[C@@H](c2ccc(OCc3ccccc3)nc2)C1 ; Cc1cnc(C(=O)C23CC(C(F)(F)F)(C2)C3)c(NC(=O)[C@H]2CCO[C@@H](c3ccc(OCc4ccccc4)nc3)C2)n1]

To a stirring solution of (2R,4S)-2-(6-benzyloxy-3-pyridyl)tetrahydropyran-4-carboxylic acid (Int-F7, 1.05 equiv., 1.12 g, 3.58 mmol) in anhydrous DCM (40 mL) at 0° C., was added DMF (0.05 mL) and oxalyl chloride (1.2 equiv., 0.36 mL, 4.09 mmol). The reaction mixture was stirred at r.t. for 1 h and concentrated in vacuo to a solid. Fresh, anhydrous DCM (20 mL) was added, and the resulting solution was stirred at 0° C. under argon gas. A separate solution of (3-amino-5-methylpyrazin-2-yl)(3-(trifluoromethyl) bicyclo[1.1.1]pentan-1-yl) methanone (Int-C22, 1 equiv., 0.93 g, 3.4 mmol) and pyridine (3 equiv., 0.83 mL, 10.2 mmol) in anhydrous DCM (20 mL) was added to the reaction mixture at 0° C. The reaction mixture was stirred at r.t. for 18 h. The reaction mixture was concentrated in vacuo and diluted with EtOAc (150 mL). The resulting solution was washed with sat. ammonium chloride (aq), sat. NaHCO₃(aq) and brine. The organic layer was dried over anhydrous Na₂SO₄, filtered, and concentrated in vacuo onto Celite (20 g). The crude mixture was purified by silica gel flash-column chromatography using an elution gradient of 10%-35% acetone in hexanes to yield (2R,4S)-2-(6-(benzyloxy)pyridin-3-yl)-N-(6-methyl-3-(3-(trifluoromethyl) bicyclo[1.1.1]pentane-1-carbonyl) pyrazin-2-yl)tetrahydro-2H-pyran-4-carboxamide (Int-C22, 1.54 g, 2.72 mmol, 63% yield) as a foam. ESI-MS: [M+H]⁺=567.3. ¹H NMR (CDCl₃, 400 MHz): δ 11.21 (1H, s), 8.23 (1H, s), 8.15 (1H, d, J=2.4 Hz), 7.65 (1H, dd, J=8.6, 2.4 Hz), 7.45 (2H, d, J=7.5 Hz), 7.31-7.39 (3H, m), 6.81 (1H, d, J=8.6 Hz), 5.38 (2H, s), 4.40 (1H, dd, J=11.4, 2.0 Hz), 4.27 (1H, dt, J=11.5, 3.0 Hz), 3.67-3.73 (1H, m), 2.95-3.04 (1H, m), 2.65 (3H, s), 2.51 (6H, s), 2.16 (1H, d, J=12.2 Hz), 1.98-2.03 (2H, m), 1.90 (1H, dd, J=24.6, 12.2 Hz).

An analogous procedure was followed to obtain the following intermediates.

| Structure | Starting Material | Characterization |
|---|---|---|
| 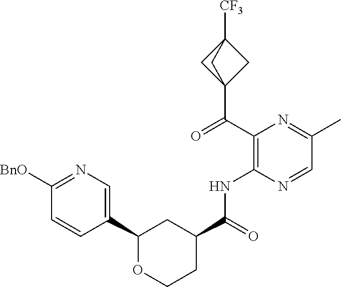 [SMILES: Cc1cnc(NC(=O)[C@H]2CCO[C@@H](c3ccc(OCc4ccccc4)nc3)C2)c(C(=O)C23CC(C(F)(F)F)(C2)C3)n1] Int-C33 | 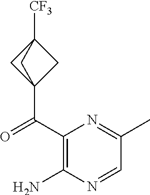 Int-C32 | The procedure yielded (2R,4S)-2-(6- (benzyloxy)pyridin-3-yl)-N-(5-methyl- 3-(3-(trifluoromethyl)- bicyclo[1.1.1]pentane-1- carbonyl)pyrazin-2-yl)tetrahydro-2H- pyran-4-carboxamide (Int-C33, 1.54 g, 2.72 mmol, 63% yield) as a foam. ESI- MS: [M + H]⁺ = 567.4. ¹H NMR (400 MHz, CDCl₃): δ 11.05 (1H, s), 8.49 (1H, s), 8.16 (1H, d, J = 2.4 Hz), 7.66 (1H, dd, J = 8.6, 2.4 Hz), 7.45 (2H, d, J = 7.5 Hz), 7.29-7.39 (3H, m), 6.81 (1H, d, J = 8.6 Hz), 5.38 (2H, s), 4.40 (1H, d, J = 11.3 Hz), 4.27 (1H, dt, J = 11.5, 3.0 Hz), 3.67- 3.73 (1H, m), 2.88-2.94 (1H, m), 2.59 (3H, s), 2.53 (6H, s), 2.13-2.18 (1H, m), 1.98-2.03 (2H, m), 1.89 (1H, dd, J = 24.6, 12.2 Hz). |
| 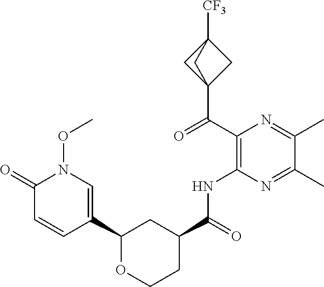 [SMILES: COn1cc([C@H]2C[C@@H](C(=O)Nc3nc(C)c(C)nc3C(=O)C34CC(C(F)(F)F)(C3)C4)CCO2)ccc1=O] Int-C45 | 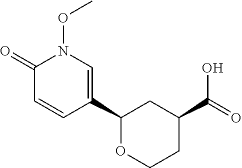 Int-C44 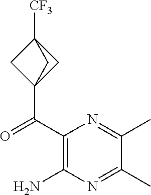 Int-F3 | Modifications: Int-C44 and Int-F3 were reacted in the presence of 1.1 equiv. oxalyl chloride for 72 h. The reaction crude was diluted with EtOAc and washed with 5% citric acid (aq.) and brine. The crude mixture was purified by flash column chromatography (12 g SiO₂ column) using an elution gradient of 0- 10% MeOH in DCM to yield (2R,4S)-N- [5,6-dimethyl-3-[3- (trifluoromethyl)bicyclo[1.1.1]pentane- 1-carbonyl]pyrazin-2-yl]-2-(1-methoxy- 6-oxo-3-pyridyl)tetrahydropyran-4- carboxamide (Int-C45, 48 mg, 0.09 mmol, 46% yield) as a solid. ESI-MS: [M + H]⁺ = 521.3. ¹H NMR (CDCl₃, 400 MHz): δ 11.10 (s, 1H), 7.58 (d, J = 2.5 Hz, 1H), 7.33 (dd, J = 9.6, 2.5 Hz, 1H), 6.68 (d, J = 9.6 Hz, 1H), 4.26-4.19 (m, 2H), 4.08 (s, 3H), 3.65 (td, J = 11.4, 3.6 Hz, 1H), 2.93-2.93 (m, 1H), 2.62 (s, 3H), 2.56 (s, 3H), 2.52 (s, 6H), 2.18-2.14 (m, 1H), 1.99-1.93 (m, 2H), 1.80 (dd, J = 25.0, 11.8 Hz, 1H). ¹⁹F NMR (CDCl₃, 376 MHz): δ −73.1 (s, 3F). |
| 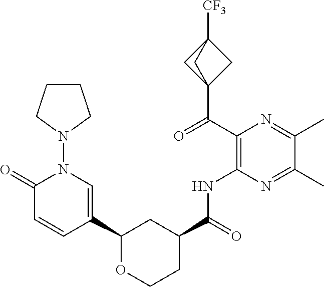 [SMILES: Cc1nc(NC(=O)[C@H]2CCO[C@@H](c3ccc(=O)n(N4CCCC4)c3)C2)c(C(=O)C23CC(C(F)(F)F)(C2)C3)nc1C] Int-D8 | 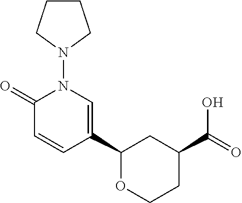 [SMILES: O=C(O)[C@H]1CCO[C@@H](c2ccc(=O)n(N3CCCC3)c2)C1] Int-D7 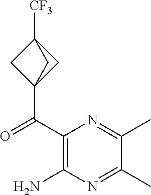 Int-F3 | Modifications: Int-D7 (1 equiv.) and Int- F3 (1.1 equiv.) were reacted in the presence of 1.02 equiv. oxalyl chloride for 16 h. The reaction mixture was concentrated under reduced pressure and then purified by flash column chromatography (12 g SiO₂ column) using an elution gradient of 10-50% EtOAc/EtOH (1:1) in hexanes to yield (2R,4S)-N-[5,6-dimethyl-3-[3- (trifluoromethyl)bicyclo[1.1.1]pentane- 1-carbonyl]pyrazin-2-yl]-2-(6-oxo-1- pyrrolidin-1-yl-3- pyridyl)tetrahydropyran-4-carboxamide (Int-D8, 95 mg, 0.17 mmol, 27% yield) as a solid. ESI-MS: [M + H]⁺ = 560.4. ¹H NMR (CDCl₃, 400 MHz): δ 11.08 (s, 1H), 7.58 (s, 1H), 7.35 (dd, J = 9.4, 2.5 Hz, 1H), 6.58 (d, J = 9.4 Hz, 1H), 4.25- 4.20 (m, 1H), 4.17 (d, J = 11.2 Hz, 1H), 3.74 (d, J = 6.2 Hz, 1H), 3.65 (dd, J = 14.9, 11.7 Hz, 1H), 3.42 (t, J = 6.3 Hz, 4H), 2.93 (d, J = 29.5 Hz, 1H), 2.62 (s, 3H), 2.56 (s, 3H), 2.52 (s, 6H), 2.13 (d, J = 13.5 Hz, 1H), 1.99-1.84 (m, 6H). ¹⁹F NMR (CDCl₃, 376 MHz): δ −73.1 (s, 3F). |
| 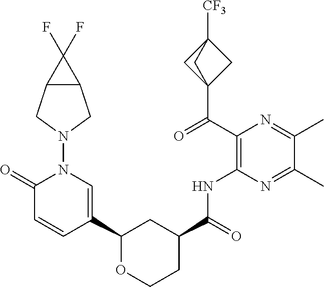 [SMILES: Cc1nc(NC(=O)[C@H]2CCO[C@@H](c3ccc(=O)n(N4CC5C(C4)C5(F)F)c3)C2)c(C(=O)C23CC(C(F)(F)F)(C2)C3)nc1C] Int-D11 | 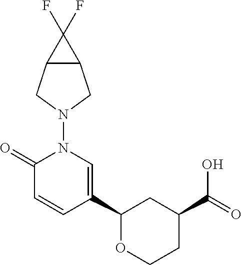 [SMILES: O=C(O)[C@H]1CCO[C@@H](c2ccc(=O)n(N3CC4C(C3)C4(F)F)c2)C1] Int-D10 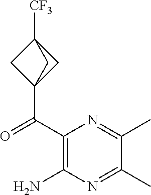 [SMILES: Cc1nc(N)c(C(=O)C23CC(C(F)(F)F)(C2)C3)nc1C] Int-F3 | Modifications: Int-D10 (1 equiv.) and Int-F3 (1.1 equiv.) were reacted in the presence of 1.1 equiv. oxalyl chloride for 16 h. The reaction mixture was concentrated under reduced pressure and then purified by flash column chromatography (12 g SiO₂ column) using an elution gradient of 0-5% MeOH in DCM to yield (2R,4S)-2-[1-(2,5- dihydropyrrol-1-yl)-6-oxo-3-pyridyl]- N-[5,6-dimethyl-3-[3- (trifluoromethyl)bicyclo[1.1.1]pentane- 1-carbonyl]pyrazin-2- yl]tetrahydropyran-4-carboxamide (Int- D11, 20.0 mg, 0.034 mmol, 16% yield) as a solid. |
| 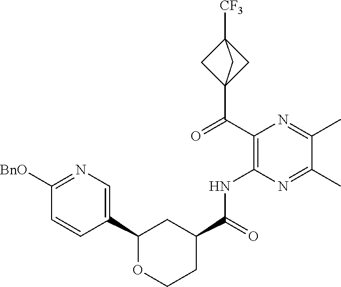 [SMILES: Cc1nc(NC(=O)[C@H]2CCO[C@@H](c3ccc(OCc4ccccc4)nc3)C2)c(C(=O)C23CC(C(F)(F)F)(C2)C3)nc1C] Int-F8 | 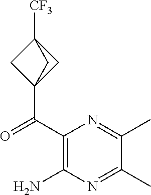 Int-F3 | The crude mixture was purified by normal phase chromatography (20-80% EtOAc in hexanes) to afford (2R,4S)-2- (6-(benzyloxy)pyridin-3-yl)-N-(5,6- dimethyl-3-(3- (trifluoromethyl)bicyclo[1.1.1]pentane- 1-carbonyl)pyrazin-2-yl)tetrahydro-2H- pyran-4-carboxamide as a solid (Int-F8, 560 mg, 43% yield). ¹H NMR (CD₃OD, 400 MHz): δ 8.13 (1H, d, J = 2.5 Hz), 7.72 (1H, dd, J = 8.6, 2.5 Hz), 7.42 (2H, d, J = 7.6 Hz), 7.31 (3H, d, J = 10.4 Hz), 6.84 (1H, d, J = 8.6 Hz), 5.33 (2H, s), 5.28 (1H, d, J = 5.3 Hz), 4.45 (1H, d, J = 11.2 Hz), 4.17-4.21 (1H, m), 3.68-3.74 (1H, m), 2.99 (1H, d, J = 13.4 Hz), 2.70 (1H, t, J = 5.8 Hz), 2.55 (6H, d, J = 1.9 Hz), 2.47 (6H, s), 1.89 (2H, s). ESI-MS: [M + H]⁺ = 581.3. |

### Method Int 79. Intermediate Int-C25: tert-butyl 3-(6-benzyloxy-3-pyridyl)-4-oxo-piperidine-1-carboxylate

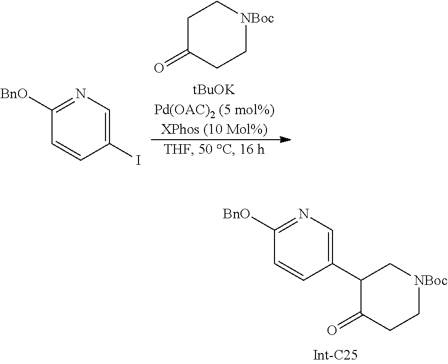 [SMILES: CC(C)(C)OC(=O)N1CCC(=O)CC1 ; Ic1ccc(OCc2ccccc2)nc1 ; CC(C)(C)OC(=O)N1CCC(=O)C(c2ccc(OCc3ccccc3)nc2)C1]

1-Boc-4-piperidinone (1.5 equiv., 0.96 g, 4.82 mmol) and 2-benzyloxy-5-iodo-pyridine 1 (1 equiv., 1.0 g, 3.21 mmol) were added to sodium tert-butoxide (3 equiv., 0.93 g, 9.64 mmol) in THF (32 mL). The mixture was flushed with argon, and then XPhos (10 mol %, 153 mg, 0.32 mmol) and Pd(OAc)₂ (5 mol %, 36 mg, 0.16 mmol) were added. The resulting mixture was stirred at 50° C. overnight, quenched with water, and extracted three times with DCM. The combined organic layers were washed with brine, dried over Na₂SO₄, filtered, and concentrated under reduced pressure to afford tert-butyl 3-(6-benzyloxy-3-pyridyl)-4-oxo-piperidine-1-carboxylate (Int-C25, 780 mg, 2.04 mmol, 63% yield) as an oil. ESI-MS: [M+H]⁺=383.4. ¹H NMR (CDCl₃, 400 MHz): δ 7.92 (1H, d, J=2.4 Hz), 7.38 (2H, d, J=7.5 Hz), 7.22-7.35 (4H, m), 6.73 (1H, d, J=8.6 Hz), 5.31 (2H, s), 4.19 (1H, br s), 3.58-3.62 (1H, m), 3.28-3.35 (1H, m), 2.32-2.56 (2H, m), 1.44 (9H, s), 1.41 (2H, s).

### Method Int 80. Intermediate Int-C26: tert-butyl 3-(6-benzyloxy-3-pyridyl)-4,4-difluoro-piperidine-1-carboxylate

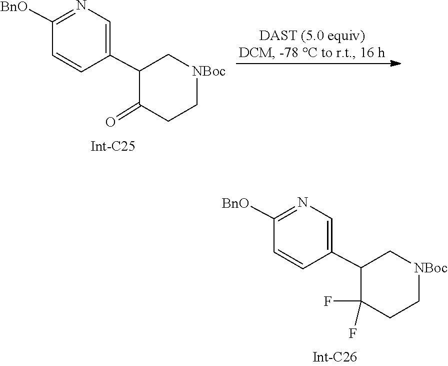 [SMILES: CC(C)(C)OC(=O)N1CCC(=O)C(c2ccc(OCc3ccccc3)nc2)C1 ; CC(C)(C)OC(=O)N1CCC(F)(F)C(c2ccc(OCc3ccccc3)nc2)C1]

DAST (5 equiv., 0.29 mL, 2.22 mmol) was added dropwise to a stirring solution of tert-butyl 3-(6-benzyloxy-3-pyridyl)-4-oxo-piperidine-1-carboxylate (Int-C25, 1 equiv., 170 mg, 0.44 mmol) in DCM (5 mL) at −78° C. The resulting mixture was allowed to warm up to room temperature overnight. The crude was concentrated under reduced pressure and purified by normal phase chromatography (0% to 30% EtOAc in hexanes) to afford tert-butyl 3-(6-benzyloxy-3-pyridyl)-4,4-difluoro-piperidine-1-carboxylate (Int-C26, 70 mg, 0.17 mmol, 39% yield) as an oil. ESI-MS: [M+H]⁺=405.3. ¹H NMR (CDCl₃, 400 MHz): δ 8.10 (1H, d, J=2.3 Hz), 7.55 (1H, d, J=8.7 Hz), 7.46 (2H, d, J=7.5 Hz), 7.38 (2H, t, J=7.4 Hz), 7.32 (1H, d, J=7.1 Hz), 6.80 (1H, d, J=8.6 Hz), 5.37-5.38 (2H, m), 4.22 (2H, br s), 2.96-3.21 (3H, m), 2.14 (1H, t, J=9.9 Hz), 1.92-2.01 (1H, m), 1.47 (9H, s).

An analogous procedure was followed to obtain the following intermediates.

| Structure | Starting Material | Characterization |
|---|---|---|
| 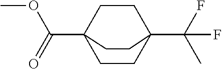 [SMILES: COC(=O)C12CCC(C(C)(F)F)(CC1)CC2] Int-F11 | 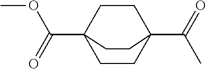 Int-F10 | The procedure afforded methyl 4- (1,1- difluoroethyl)bicyclo[2.2.1]heptane- 1-carboxylate (Int-F11). |

### Method Int 81. Intermediate Int-C27:5-(4,4-difluoropiperidin-3-yl)pyridin-2 (1H)-one

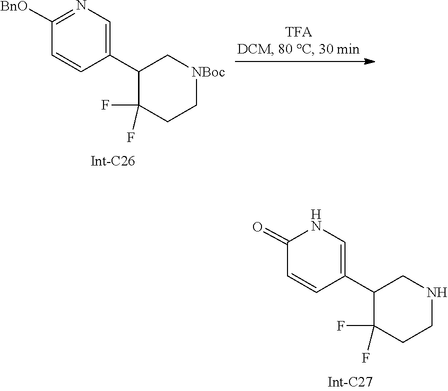 [SMILES: CC(C)(C)OC(=O)N1CCC(F)(F)C(c2ccc(OCc3ccccc3)nc2)C1 ; O=c1ccc(C2CNCCC2(F)F)c[nH]1]

TFA (90 equiv., 1.2 mL, 15.6 mmol) was added to a solution of tert-butyl 3-(6-benzyloxy-3-pyridyl)-4,4-difluoro-piperidine-1-carboxylate (Int-C26, 1 equiv., 70 mg, 0.17 mmol) in DCM (1.7 mL) at room temperature. The resulting mixture was stirred at 80° C. for 30 min in a microwave vial. The reaction was cooled down to room temperature, and the volatiles were removed under reduced pressure. The residue was co-evaporated with DCM three times to afford crude 5-(4,4-difluoropiperidin-3-yl)pyridin-2 (1H)-one (Int-C27, 37 mg, 0.17 mmol, 99% yield) as an oil. The crude mixture was used without further purification in the next step.

An analogous procedure was followed to obtain the following intermediates.

| Structure | Starting Material | Characterization |
|---|---|---|
| 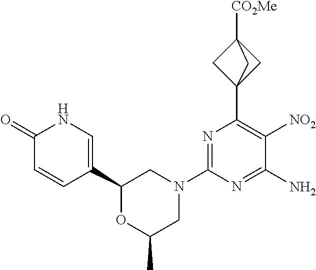 [SMILES: COC(=O)C12CC(c3nc(N4C[C@@H](C)O[C@@H](c5ccc(=O)[nH]c5)C4)nc(N)c3[N+](=O)[O-])(C1)C2] Int-C48 | 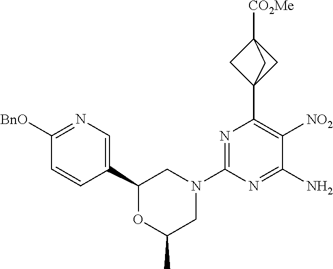 Int-C47 | Modification: The reaction was run for 1 h at 80 °C to afford methyl 3-[6-amino-2- [(2R,6S)-2-methyl-6-(6- oxo-1H-pyridin-3- yl)morpholin-4-yl]-5-nitro- pyrimidin-4- yl]bicyclo[1.1.1]pentane-1- carboxylate (Int-C48, 438 mg, 0.96 mmol, 99% yield). ESI-MS: [M + H]⁺ = 457.3. |

### Method Int 82. Intermediate Int-C28:5-(4-(5-(2,4-difluorophenyl)-2,3-dimethylpyrido[3,4-b]pyrazin-7-yl)-5,6-dihydro-2H-pyran-2-yl)-1-methylpyridin-2 (1H)-one

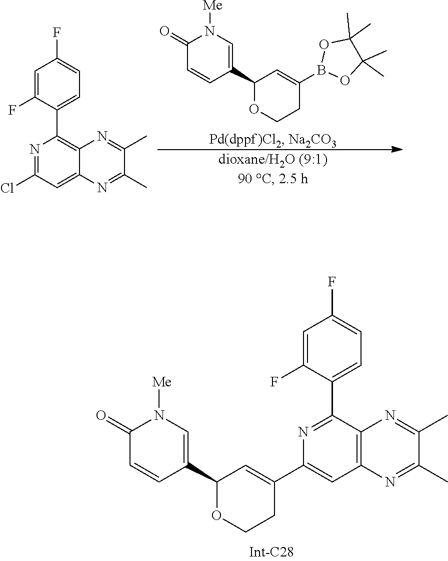 [SMILES: Cc1nc2cc(Cl)nc(-c3ccc(F)cc3F)c2nc1C ; Cn1cc([C@H]2C=C(B3OC(C)(C)C(C)(C)O3)CCO2)ccc1=O ; Cc1nc2cc(C3=C[C@H](c4ccc(=O)n(C)c4)OCC3)nc(-c3ccc(F)cc3F)c2nc1C]

To a glass vial equipped with a Teflon-coated magnetic stirring bar was added 1-methyl-5-[4-(4,4,5,5-tetramethyl-1,3,2-dioxaborolan-2-yl)-3,6-dihydro-2H-pyran-6-yl]pyridin-2-one (1.15 equiv., 170 mg, 0.37 mmol), 7-chloro-5-(2,4-difluorophenyl)-2,3-dimethyl-pyrido[3,4-b]pyrazine (1 equiv., 100 mg, 0.33 mmol), Pd(dppf)Cl₂·DCM (0.15 equiv., 40 mg, 0.49 mmol), 1,4-dioxane (3.3 mL) and 1.0 M Na₂CO₃ (aq.)(3 equiv., 0.50 mL, 0.99 mmol). The vial was sealed, purged under argon, and stirred at 90° C. for 2.5 h. The reaction was then cooled to r.t., filtered over a pad of Celite and rinsed with EtOAc. The filtrate was concentrated under reduced pressure and purified by flash chromatography (Isco RediSep® column 12 g, using an elution gradient from 0.5% to 2% MeOH in DCM) to afford 5-(4-(5-(2,4-difluorophenyl)-2,3-dimethylpyrido[3,4-b]pyrazin-7-yl)-5,6-dihydro-2H-pyran-2-yl)-1-methylpyridin-2 (1H)-one (Int-C28, 59 mg, 0.13 mmol, 87% yield) as a solid. ESI-MS: [M+H]⁺=461.4. ¹H NMR (CDCl₃, 400 MHz): δ 7.84 (s, 1H), 7.67 (q, J=7.6 Hz, 1H), 7.42 (d, J=9.3 Hz, 1H), 7.35 (s, 1H), 7.00-7.05 (m, 2H), 6.95, (t, J=9.5 Hz, 1H), 6.59 (d, J=9.0 Hz, 1H), 5.17 (s, 1H), 4.19 (d, J=11.0 Hz, 1H), 3.90-4.03 (m, 2H), 3.53 (s, 3H), 2.76 (s, 3H), 2.68 (s, 3H), 1.84 (br s, 1H).

An analogous procedure was followed to obtain the following intermediates.

| Structure | Starting Material | Characterization |
|---|---|---|
| 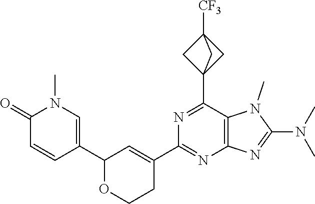 [SMILES: CN(C)c1nc2nc(C3=CC(c4ccc(=O)n(C)c4)OCC3)nc(C34CC(C(F)(F)F)(C3)C4)c2n1C] Int-C40 | 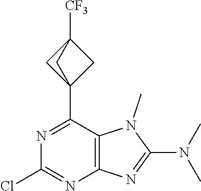 [SMILES: CN(C)c1nc2nc(Cl)nc(C34CC(C(F)(F)F)(C3)C4)c2n1C] Int-C39 | Modification: The reaction was run in 4:1 dioxane/H₂O mixed solvent, using 2 equiv. K₂CO₃ (solid) as base reagent. The reaction mixture was heated to 105 ºC for 16 h. The crude was purified by silica gel flash column chromatography using an elution gradient of 40-100% acetone in hexanes to yield 5-(4-(8- (dimethylamino)-7-methyl-6-(3- (trifluoromethyl)bicyclo[1.1.1]pentan- 1-yl)-7H-purin-2-yl)-5,6-dihydro-2H- pyran-2-yl)-1-methylpyridin-2(1H)- one (Int-C40, 195 mg, 0.39 mmol, |
| | | 99% yield) as an oil. ESI-MS: [M + H]⁺ = |
| | | 501.4. ¹H NMR (CDCl₃, 400 MHz): |
| | | δ 7.44 (1H, s), 7.42 (1H, s), 6.59 (1H, |
| | | d, J = 9.4 Hz), 5.16 (1H, s), 4.06-4.10 |
| | | (1H, m), 3.84-3.88 (1H, m), 3.75 (3H, |
| | | s), 3.55 (3H, s), 3.21 (7H, s), 2.83 (2H, |
| | | s), 2.63 (1H, s), 2.54 (6H, s). |
| 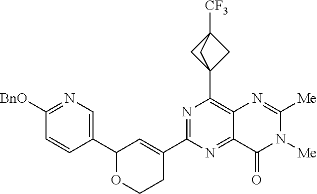 [SMILES: Cc1nc2c(C34CC(C(F)(F)F)(C3)C4)nc(C3=CC(c4ccc(OCc5ccccc5)nc4)OCC3)nc2c(=O)n1C] Int-D15 | 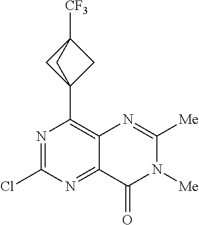 [SMILES: Cc1nc2c(C34CC(C(F)(F)F)(C3)C4)nc(Cl)nc2c(=O)n1C] Int-D14 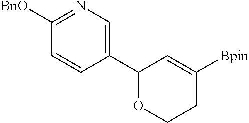 | Modification: The reaction was run with Int-D14 and 2-benzyloxy-5-[rac- (6R)-4-(4,4,5,5-tetramethyl-1,3,2- dioxaborolan-2-yl)-3,6-dihydro-2H- pyran-6-yl]pyridine (1.3 equiv.) and utilized Cs₂CO₃ (solid) as base. The reaction crude was purified by flash chromatography (12 g SiO₂ column) using an elution gradient of 20-80% EtOAc in hexanes to yield 2,3- dimethyl-6-[rac-(6R)-6-(6-benzyloxy- 3-pyridyl)-3,6-dihydro-2H-pyran-4- yl]-8-[3-(trifluoromethyl)-1- bicyclo[1.1.1]pentanyl]pyrimido[5,4- d]pyrimidin-4-one (Int-D15, 73 mg, 0.13 mmol, 87% yield) as a solid. ESI- MS: [M + H]⁺ = 576.3. ¹H NMR (CDCl₃, 400 MHz): δ 8.21 (d, J = 2.3 Hz, 1H), 7.65 (dd, J = 8.6, 2.4 Hz, 1H), 7.47-7.44 (m, 2H), 7.39-7.35 (m, 3H), 7.33-7.29 (m, 1H), 6.81 (d, J = 8.7 Hz, 1H), 5.38 (d, J = 3.9 Hz, 3H), 4.18 (ddd, J = 11.3, 4.7, 3.9 Hz, 1H), |
| | | 3.93-3.87 (m, 1H), 3.65 (s, 3H), 2.99 |
| | | 2.85 (m, 2H), 2.65 (s, 3H), 2.56 (s, 6H). |
| | | ¹⁹F NMR (CDCl₃, 376 MHz): δ −73.0 |
| | | (s, 3F) |
| 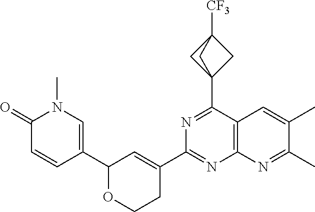 [SMILES: Cc1cc2c(C34CC(C(F)(F)F)(C3)C4)nc(C3=CC(c4ccc(=O)n(C)c4)OCC3)nc2nc1C] Int-E4 | 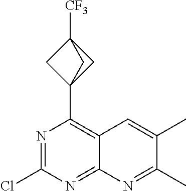 Int-E3 | Modifications: The reaction was run in 7:1 dioxane/H₂O with for 1.5 h and purified by flash chromatography (Isco RediSep ® column 4 g) (0-3% MeOH in DCM) to afford 5-[4-[6,7-dimethyl- 4-[3-(trifluoromethyl)-1- bicyclo[1.1.1]pentanyl]pyrido[2,3- d]pyrimidin-2-yl]-3,6-dihydro-2H- pyran-6-yl]-1-methyl-pyridin-2-one (Int-E3, 39 mg, 81.2 umol, 59% yield) as an oil. ESI-MS: [M + H]⁺ = 483.4. |
| 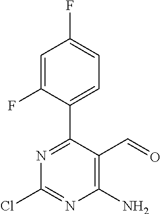 [SMILES: Nc1nc(Cl)nc(-c2ccc(F)cc2F)c1C=O] Int-E15 | 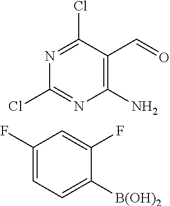 | The residue was purified by column chromatography (220 g SiO₂) using a 0-50 % EtOAc in DCM gradient to afford 4-amino-2-chloro-6-(2,4- difluorophenyl)pyrimidine-5- carbaldehyde (Int-E15, 2.75 g, 10.2 mmol, 39%). |
| 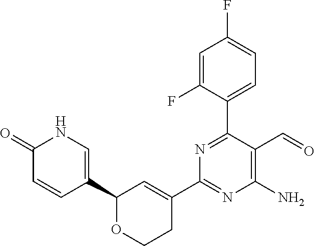 [SMILES: Nc1nc(C2=C[C@H](c3ccc(=O)[nH]c3)OCC2)nc(-c2ccc(F)cc2F)c1C=O] Int-E16 | 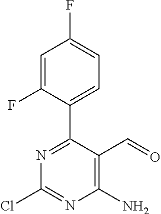 [SMILES: Nc1nc(Cl)nc(-c2ccc(F)cc2F)c1C=O] Int-E15 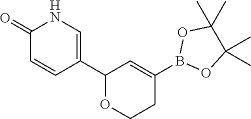 | The residue was purified on a 120 g silica column using a 0-20% MeOH in DCM gradient to yield (R)-4-amino-6- (2,4-difluorophenyl)-2-(6-(6-oxo-1,6- dihydropyridin-3-yl)-3,6-dihydro-2H- pyran-4-yl)pyrimidine-5-carbaldehyde (Int-E16, 480 mg, 1.17 mmol, 57%) as a solid. ESI-MS: [M + H]⁺ = 411.2. |
| 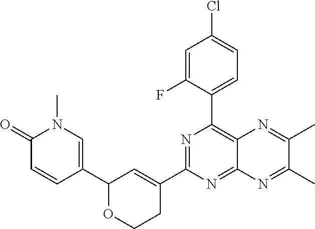 [SMILES: Cc1nc2nc(C3=CC(c4ccc(=O)n(C)c4)OCC3)nc(-c3ccc(Cl)cc3F)c2nc1C] Int-E44 | 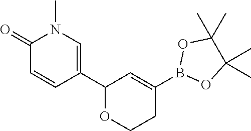 Int-E43 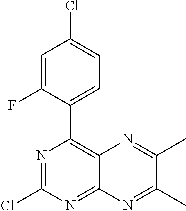 | Modification: The reaction was run with 2 equiv. K₂CO₃ in 10:1 dioxane/H₂O at 100 °C for 12 h and then purified by flash column chromatography (first 0-100% EtOAc in PE, then 0-50% MeOH in MeOH) to give 5-[4-[4-(4-chloro-2-fluoro- phenyl)-6,7-dimethyl-pteridin-2-yl]- 3,6-dihydro-2H-pyran-6-yl]-1-methyl- pyridin-2-one (Int-E44, 124 mg, 0.259 mmol, 84% yield) as an oil. ESI-MS: [M + H]⁺ = 478.1. ¹H NMR (400 MHz, CDCl₃) δ 7.71 (t, J = 7.8 Hz, 1H), 7.53 (d, J = 1.9 Hz, 1H), 7.43 (dd, J = 2.4, 9.4 Hz, 1H), 7.37-7.33 (m, 2H), 7.32-7.27 (m, 1H), 6.61 (d, J = 9.4 Hz, 1H), 5.22 (br d, J = 2.5 Hz, 1H), 4.20 (td, J = 4.5, 11.3 Hz, 1H), 3.95-3.90 (m, 1H), 3.55 (s, 3H), 3.00 (br d, J = 2.3 Hz, 2H), 2.85 (s, 3H), 2.74 (s, 3H). ¹⁹F NMR (377 MHz, CDCl₃) δ −108.44 (s, 1F). |

### Method Int 83. Intermediate Int-C29:5-(4-(5-(2,4-difluorophenyl)-2,3-dimethyl-1,2,3,4-tetrahydropyrido[3,4-b]pyrazin-7-yl)tetrahydro-2H-pyran-2-yl)-1-methylpyridin-2 (1H)-one

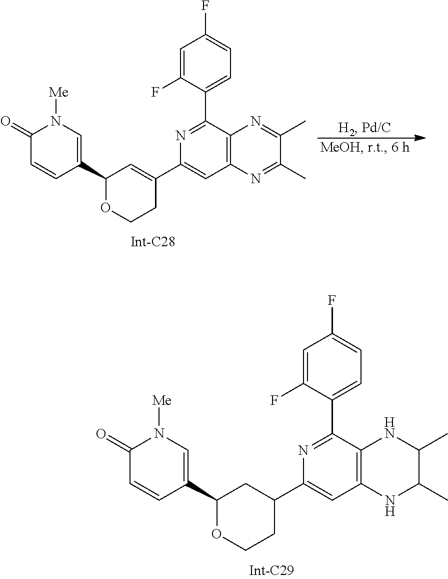 [SMILES: Cc1nc2cc(C3=C[C@H](c4ccc(=O)n(C)c4)OCC3)nc(-c3ccc(F)cc3F)c2nc1C ; CC1Nc2cc(C3CCO[C@@H](c4ccc(=O)n(C)c4)C3)nc(-c3ccc(F)cc3F)c2NC1C]

To a flame-dried microwave vial equipped with a Teflon-coated magnetic stirring bar was added (R)-5-(4-(5-(2,4-difluorophenyl)-2,3-dimethylpyrido[3,4-b]pyrazin-7-yl)-5,6-dihydro-2H-pyran-2-yl)-1-methylpyridin-2 (1H)-one (Int-C28, 1 equiv., 103 mg, 0.22 mmol) and Pd/C (10 wt %, wetted with ~55% water)(0.4 equiv., 10 mg, 0.94 mmol) under an argon atmosphere. MeOH (1.6 mL) was added, and three vacuum/hydrogen cycles were performed. The vessel was fitted with a H₂ balloon, and the reaction mixture was stirred under 1 atm of H₂ for 6 h. The reaction mixture was then purged under argon, filtered over a plug of Celite, and washed with 9:1 EtOAc:MeOH (100 mL). The filtrate was concentrated under reduced pressure and the crude mixture was purified by flash column chromatography (4 g SiO₂ column, liquid loading) using an elution gradient from 5% to 10% MeOH in DCM to afford 5-(4-(5-(2,4-difluorophenyl)-2,3-dimethyl-1,2,3,4-tetrahydropyrido[3,4-b]pyrazin-7-yl)tetrahydro-2H-pyran-2-yl)-1-methylpyridin-2 (1H)-one (Int-C29, 40 mg, 0.87 mmol, 39% yield) as a solid. ESI-MS: [M+H]⁺=463.3.

An analogous procedure was followed to obtain the following intermediates.

| Structure | Starting Material | Characterization |
|---|---|---|
| 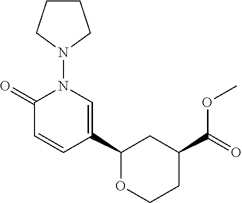 [SMILES: COC(=O)[C@H]1CCO[C@@H](c2ccc(=O)n(N3CCCC3)c2)C1] Int-D6 | 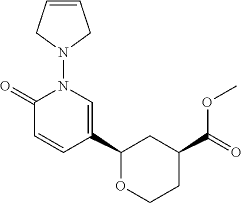 [SMILES: COC(=O)[C@H]1CCO[C@@H](c2ccc(=O)n(N3CC=CC3)c2)C1] Int-D5 | Modifications: The reaction was run with 0.03 equiv. Pd/C (10 wt %, not pre-wetted) and EtOH solvent for 1 h to afford methyl (2R,4S)-2-(6- oxo-1-pyrrolidin-1-yl-3- pyridyl)tetrahydropyran-4-carboxylate (Int-D6, 240 mg, 0.78 mmol, 96% yield) as a solid. ESI- MS: [M + H]⁺ = 307.4. ¹H NMR (CDCl₃, 400 MHz): δ 7.53 (d, J = 2.5 Hz, 1H), 7.31 (dd, J = 9.4, 2.5 Hz, 1H), 6.55 (d, J = 9.4 Hz, 1H), 4.18-4.14 (m, 1H), 4.09 (dd, J = 11.5, 1.9 Hz, 1H), 3.71 (s, 3H), 3.57 (td, J = 12.1, 2.4 Hz, 1H), 3.41 (t, J = 6.5 Hz, 4H), 2.67 (tt, J = 12.3, 3.9 Hz, 1H), 2.08 (dt, J = 13.2, 1.8 Hz, 1H), 2.00-1.64 (m, 7H). |

### Method Int 84. Intermediate Int-C36:1-cyclopropyl-5-((2R,4S)-4-(4,5-diamino-6-(3-(trifluoromethyl) bicyclo[1.1.1]pentan-1-yl)pyrimidin-2-yl)tetrahydro-2H-pyran-2-yl)pyridin-2 (1H)-one

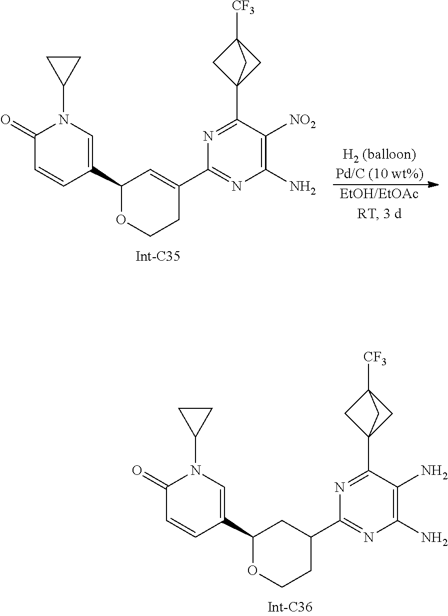 [SMILES: Nc1nc(C2=C[C@H](c3ccc(=O)n(C4CC4)c3)OCC2)nc(C23CC(C(F)(F)F)(C2)C3)c1[N+](=O)[O-] ; Nc1nc(C2CCO[C@@H](c3ccc(=O)n(C4CC4)c3)C2)nc(C23CC(C(F)(F)F)(C2)C3)c1N]

To a stirring solution of (R)-5-(4-(4-amino-5-nitro-6-(3-(trifluoromethyl) bicyclo[1.1.1]pentan-1-yl)pyrimidin-2-yl)-5,6-dihydro-2H-pyran-2-yl)-1-cyclopropylpyridin-2 (1H)-one (Int-C35, 1 equiv., 300 mg, 0.61 mmol) in EtOAc (7.0 mL) and EtOH (5.0 mL) was added Pd/C (10 wt %)(0.15 equiv., 98 mg, 92.0 μmol), at r.t. under argon atmosphere. A balloon full of hydrogen gas was attached to the reaction flask, and stirring proceeded for 3 days. The reaction mixture was then purged with argon, filtered over a short bed of Celite, and rinsed with MeOH (10 mL). The filtrate was concentrated under reduced pressure and purified, first, by silica gel flash column chromatography using an elution gradient of 1-8% MeOH in DCM, and second, by two rounds of preparative HPLC: (Condition 14, Gradient a) and then (Condition 15, Gradient a) to yield 1-cyclopropyl-5-((2R,4S)-4-(4,5-diamino-6-(3-(trifluoromethyl) bicyclo[1.1.1]pentan-1-yl)pyrimidin-2-yl)tetrahydro-2H-pyran-2-yl)pyridin-2 (1H)-one (Int-C36, 72 mg, 0.16 mmol, 25% yield) as a solid. ESI-MS: [M+H]⁺=462.3. ¹H NMR (CD₃OD, 400 MHz): δ 7.53-7.56 (2H, m), 6.51 (1H, d, J=9.1 Hz), 4.30 (1H, d, J=11.3 Hz), 4.15 (1H, dd, J=11.4, 4.3 Hz), 3.68 (1H, dd, J=12.7, 11.0 Hz), 2.90-2.96 (1H, m), 2.43 (6H, s), 1.88-1.96 (2H, m), 1.76-1.84 (2H, m), 1.11 (2H, d, J=7.3 Hz), 0.89 (2H, s).

### Method Int 85. Intermediate Int-C37:2-chloro-6-(3-(trifluoromethyl) bicyclo[1.1.1]pentan-1-yl)pyrimidine-4,5-diamine

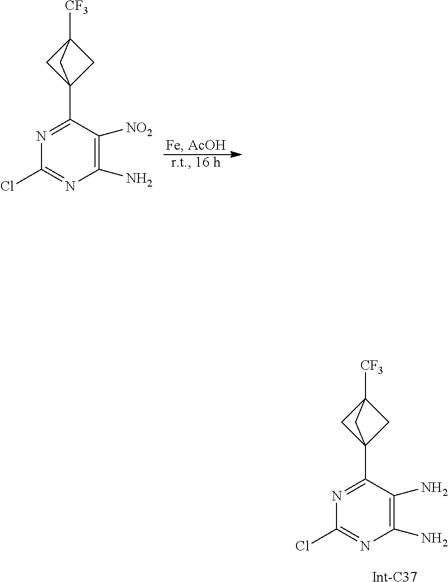

To a stirring solution of 2-chloro-5-nitro-6-(3-(trifluoromethyl) bicyclo[1.1.1]pentan-1-yl)pyrimidin-4-amine (1 equiv., 0.93 g, 3.0 mmol) in acetic acid (8.0 mL) was added iron (2.5 equiv., 0.42 g, 7.5 mmol). The reaction mixture was stirred at r.t. for 16 h, then filtered over a bed of Celite and rinsed with toluene (50 mL). The filtrate was concentrated under reduced pressure and reconstituted in EtOAc (50 mL) and H₂O (50 mL). The pH of the aqueous layer was adjusted to pH=8 using sat. NaHCO₃(aq), and the biphasic system was filtered over Celite, rinsing with EtOAc (50 mL) and H₂O (50 mL). The aqueous layer was separated and extracted three times with EtOAc (50 mL). The combined organic extracts were washed with brine, dried over anhydrous Na₂SO₄, filtered, and concentrated under reduced pressure to yield 2-chloro-6-(3-(trifluoromethyl) bicyclo[1.1.1]pentan-1-yl)pyrimidine-4,5-diamine (Int-C37, 0.83 g, 2.98 mmol, 99% yield) as a solid. ESI-MS: [M+H]⁺=279.2. ¹H NMR (400 MHz, CDCl₃): δ 2.44 (6H, s).

### Method Int 86. Intermediate Int-C38:2-chloro-N⁵-methyl-6-(3-(trifluoromethyl) bicyclo[1.1.1]pentan-1-yl)pyrimidine-4,5-diamine

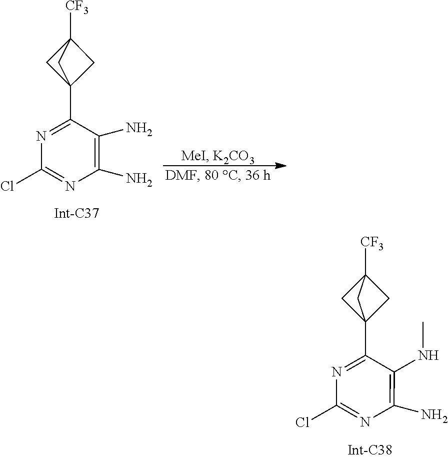 [SMILES: Nc1nc(Cl)nc(C23CC(C(F)(F)F)(C2)C3)c1N ; CNc1c(N)nc(Cl)nc1C12CC(C(F)(F)F)(C1)C2]

To a stirring solution of 2-chloro-6-(3-(trifluoromethyl) bicyclo[1.1.1]pentan-1-yl)pyrimidine-4,5-diamine (Int-C37, 1 equiv., 433 mg, 1.55 mmol) and potassium carbonate (1.2 equiv., 258 mg, 1.86 mmol) in anhydrous DMF (4 mL) at r.t., was added methyl iodide (1.3 equiv., 0.13 mL, 2.02 mmol). The reaction mixture was stirred at 80° C. for 36 h and then cooled to r.t. and diluted with EtOAc (100 mL). The resulting solution was washed with 50% sat. NaHCO₃(aq) and three times with brine. The organic layer was dried over anhydrous Na₂SO₄, filtered, and concentrated under reduced pressure. The crude mixture was purified by silica gel flash column chromatography using an elution gradient of 10-50% EtOAc in hexanes to yield 2-chloro-N⁵-methyl-6-(3-(trifluoromethyl) bicyclo[1.1.1]pentan-1-yl)pyrimidine-4,5-diamine (Int-C38, 130 mg, 0.44 mmol, 29% yield) as a solid. ESI-MS: [M+H]⁺=293.1. ¹H NMR (400 MHz, CDCl₃): δ 2.59 (3H, s), 2.43 (6H, s).

An analogous procedure was followed to obtain the following intermediates.

| Structure | Starting Material | Characterization |
|---|---|---|
| 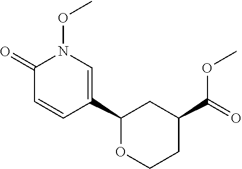 [SMILES: COC(=O)[C@H]1CCO[C@@H](c2ccc(=O)n(OC)c2)C1] Int-C43 | 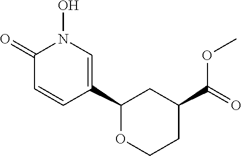 Int-C42 | Modification: The reaction was run 2 equiv. each of MeI and K₂CO₃ at r.t. for 2 h. The resulting residue was purified by flash chromatography (6 g SiO₂ column) using an elution gradient of 0-10% MeOH in DCM to yield methyl (2R,4S)-2- (1-methoxy-6-oxo-1,6-dihydropyridin-3- yl)tetrahydro-2H-pyran-4-carboxylate (Int-C43, 20 mg, 0.075 mmol, 26% yield) as an oil. ESI- MS: [M + H]⁺ = 268.2 (obs). ¹H NMR (CDCl₃, 400 MHz): δ 7.53 (d, J = 2.3 Hz, 1H), 7.28 (dd, J = 9.4, 2.5 Hz, 1H), 6.64 (d, J = 9.4 Hz, 1H), |
| | | 4.17-4.09 (m, 2H), 4.04 (s, 3H), 3.68 (s, 3H), |
| | | 3.55 (td, J = 12.1, 2.4 Hz, 1H), 2.65 (tt, J = 12.4, |
| | | 3.8 Hz, 1H), 2.11-2.05 (m, 1H), 1.89 (ddd, J = |
| | | 13.6, 3.9, 2.1 Hz, 1H), 1.74 (ddd, J = 25.4, |
| | | 12.9, 4.8 Hz, 1H), 1.60 (q, J = 12.5 Hz, 1H). |
| 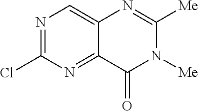 [SMILES: Cc1nc2cnc(Cl)nc2c(=O)n1C] Int-D13 | 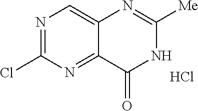 [SMILES: Cc1nc2cnc(Cl)nc2c(=O)[nH]1 ; Cl] Int-D12 | Modifications: The reaction was run with 1.67 equiv. of MeI and 2.5 equiv. of K₂CO₃ in MeCN solvent at 55 °C for 16 h, and then the mixture was filtered through Celite and concentrated under reduced pressure. The crude material was purified by flash chromatography (80 g SiO₂ column) using an elution gradient of 10-70% EtOAc: EtOH (3:1) in hexanes to yield 6-chloro- |
| | | 2,3-dimethyl-pyrimido[5,4-d]pyrimidin-4-one |
| | | (Int-D13, 1.37 g, 6.5 mmol, 83% yield) as a |
| | | powder. ¹H NMR (CDCl₃, 400 MHz): δ 9.12 (s, |
| | | 1H), 3.69 (s, 3H), 2.68 (s, 3H). ESI-MS: |
| | | [M + H]⁺ = 211.1. |
| 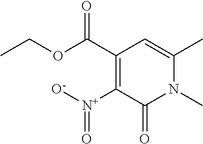 [SMILES: CCOC(=O)c1cc(C)n(C)c(=O)c1[N+](=O)[O-]] Int-D28 | 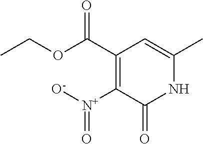 Int-D27 | Modifications: The reaction was run with 3 equiv. of MeI and 2 equiv. of K₂CO₃ in at 50 °C for 2 h. The crude was purified by column chromatography (gradient elution of hexane/EtOAc = 5:1~0:1) to obtain ethyl 1,6- dimethyl-3-nitro-2-oxo-pyridine-4-carboxylate (Int-D28, 11.00 g, 45.8 mmol, 83% yield) as a solid. LCMS: [M + H]⁺ = 241.1. ¹H NMR (400 MHz, CD₃OD) δ 6.62 (s, 1H), 4.36 (q, J = 7.2 Hz, 2H), 3.66 (s, 3H), 2.56 (s, 3H), 1.33 (t, J = 7.2 Hz, 3H) |

### Method Int 87. Intermediate Int-C39:2-chloro-N,N,7-trimethyl-6-(3-(trifluoromethyl) bicyclo[1.1.1]pentan-1-yl)-7H-purin-8-amine

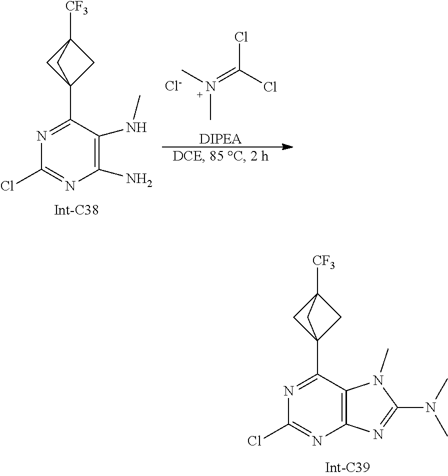

To a stirring solution of 2-chloro-N⁵-methyl-6-(3-(trifluoromethyl) bicyclo[1.1.1]pentan-1-yl)pyrimidine-4,5-diamine (Int-C38, 1 equiv., 130 mg, 0.44 mmol) in anhydrous DCE (4.0 mL) at r.t. was added dichloromethylene (dimethyl) ammonium chloride (1.2 equiv., 87 mg, 0.533 mmol) and DIPEA (3 equiv., 0.23 mL, 1.33 mmol). The reaction mixture was heated to 85° C. for 2 h and then cooled to r.t. and diluted with EtOAc. The resulting solution was washed with sat. ammonium chloride (aq) and then with brine. The organic layer was dried over anhydrous Na₂SO₄, filtered, and concentrated under reduced pressure. The crude mixture was purified by silica gel flash column chromatography using an elution gradient of 40-100% EtOAc in hexanes to yield 2-chloro-N,N,7-trimethyl-6-(3-(trifluoromethyl) bicyclo[1.1.1]pentan-1-yl)-7H-purin-8-amine (Int-C39, 140 mg, 0.41 mmol, 91% yield) as a solid. ESI-MS: [M+H]⁺=346.1. ¹H NMR (CDCl₃, 400 MHz): δ 3.71 (3H, s), 3.19 (6H, s), 2.51 (6H, s).

### Method Int 88. Intermediate Int-C41: Methyl(2R,4S)-2-(6-benzyloxy-1-oxido-pyridin-1-ium-3-yl)tetrahydropyran-4-carboxylate

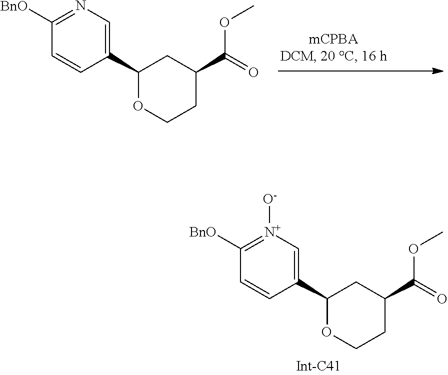 [SMILES: COC(=O)[C@H]1CCO[C@@H](c2ccc(OCc3ccccc3)nc2)C1 ; COC(=O)[C@H]1CCO[C@@H](c2ccc(OCc3ccccc3)[n+]([O-])c2)C1]

A 100 mL flame-dried round-bottomed flask equipped with a Teflon-coated magnetic stirring bar was charged with methyl(2R,4S)-2-(6-benzyloxy-3-pyridyl)tetrahydropyran-4-carboxylate (1 equiv., 1.0 g, 3.1 mmol) and anhydrous DCM (20 mL). Then mCPBA (2.5 equiv., 1.6 g, 7.6 mmol) was added at 0° C. under argon, and the reaction mixture was stirred at r.t. for 16 h. CaCO₃ (4 equiv., 1.23 g, 12.3 mmol) was added to the suspension, and the reaction mixture was stirred for 10 min before filtering through a fritted funnel and rinsing with DCM. The resulting residue was purified by flash chromatography (120 g SiO₂ column) using an elution gradient of 0.5-10% MeOH in DCM, solid loading) to yield methyl(2R,4S)-2-(6-benzyloxy-1-oxido-pyridin-1-ium-3-yl)tetrahydropyran-4-carboxylate (Int-C41, 412 mg, 1.2 mmol, 39% yield) as a solid. ESI-MS: [M+H]⁺=344.2. ¹H NMR (CDCl₃, 400 MHz): δ 8.31 (d, J=2.1 Hz, 1H), 7.45-7.33 (m, 5H), 7.15 (dd, J=8.6, 1.9 Hz, 1H), 6.81 (d, J=8.7 Hz, 1H), 5.43 (s, 2H), 4.25 (dd, J=11.5, 1.9 Hz, 1H), 4.18 (ddd, J=11.7, 4.6, 1.3 Hz, 1H), 3.67-3.70 (3H), 3.57 (td, J=12.1, 2.2 Hz, 1H), 2.69 (tt, J=12.3, 3.8 Hz, 1H), 2.12-2.06 (m, 1H), 1.91 (dd, J=13.5, 1.8 Hz, 1H), 1.76 (ddd, J=25.9, 12.5, 4.6 Hz, 1H), 1.60 (q, J=12.4 Hz, 1H).

### Method Int 89. Intermediate Int-C44: (2R,4S)-2-(1-methoxy-6-oxo-1,6-dihydropyridin-3-yl)tetrahydro-2H-pyran-4-carboxylic acid

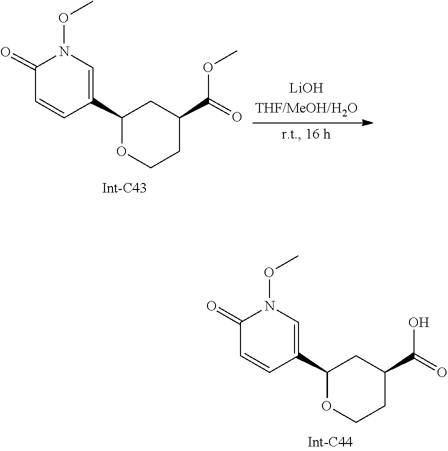 [SMILES: COC(=O)[C@H]1CCO[C@@H](c2ccc(=O)n(OC)c2)C1 ; COn1cc([C@H]2C[C@@H](C(=O)O)CCO2)ccc1=O]

A vial equipped with a Teflon-coated stirring bar was charged with methyl(2R,4S)-2-(1-methoxy-6-oxo-3-pyridyl)tetrahydropyran-4-carboxylate (Int-C43, 1 equiv., 48 mg, 0.18 mmol) and a 1:1 mixture of THF/MeOH (2 mL). LiOH (2.5 equiv., 11 mg, 0.45 mmol) was added followed by water (0.2 mL) and the reaction mixture was stirred at r.t. for 16 h. After cooling it down to 0° C., IN HCl (aq.) was added until pH=4 was obtained. The solution was then extracted with EtOAc (3×10 mL), the organic phases were combined, washed with brine (30 mL), dried over anhydrous Na₂SO₄, filtered, and concentrated under reduced pressure to yield (2R,4S)-2-(1-methoxy-6-oxo-3-pyridyl)tetrahydropyran-4-carboxylic acid (Int-C44, 28 mg, 0.11 mmol, 61% yield) as a solid. ESI-MS: [M+H]⁺254.2. ¹H NMR (CDCl₃, 400 MHz): δ 7.58 (d, J=2.1 Hz, 1H), 7.33 (dd, J=9.4, 2.5 Hz, 1H), 6.73 (d, J=9.4 Hz, 1H), 4.20-4.14 (m, 2H), 4.07 (s, 3H), 3.59 (td, J=12.0, 2.0 Hz, 1H), 2.74-2.68 (m, 1H), 2.14 (d, J=13.0 Hz, 1H), 1.97-1.94 (m, 1H), 1.78 (dd, J=13.0, 4.6 Hz, 1H), 1.63 (q, J=12.3 Hz, 1H).

### Method Int 90. Intermediate Int-C49: Methyl 3-[6-amino-2-[(2R,6S)-2-methyl-6-(1-methyl-6-oxo-3-pyridyl) morpholin-4-yl]-5-nitro-pyrimidin-4-yl]bicyclo[1.1.1]pentane-1-carboxylate

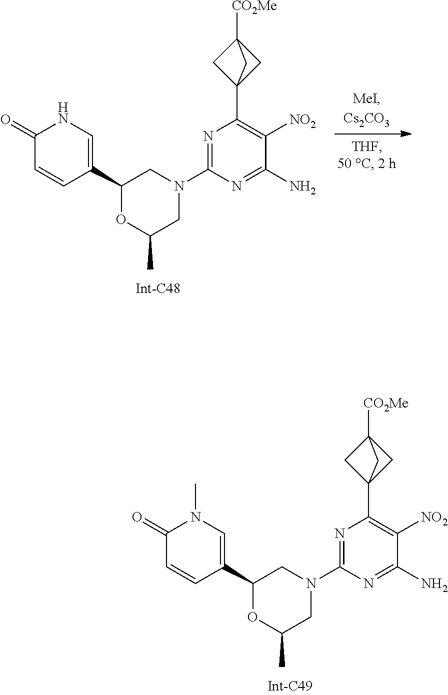 [SMILES: COC(=O)C12CC(c3nc(N4C[C@@H](C)O[C@@H](c5ccc(=O)[nH]c5)C4)nc(N)c3[N+](=O)[O-])(C1)C2 ; COC(=O)C12CC(c3nc(N4C[C@@H](C)O[C@@H](c5ccc(=O)n(C)c5)C4)nc(N)c3[N+](=O)[O-])(C1)C2]

A mixture of methyl 3-[6-amino-2-[(2R,6S)-2-methyl-6-(6-oxo-1H-pyridin-3-yl) morpholin-4-yl]-5-nitro-pyrimidin-4-yl]bicyclo[1.1.1]pentane-1-carboxylate (Int-C48, 1 equiv., 438 mg, 0.96 mmol), methyl iodide (2 equiv., 0.12 mL, 1.92 mmol) and cesium carbonate (3 equiv., 938 mg, 2.88 mmol) was stirred in THF (10 mL) at 50° C. for 2 h. The reaction was quenched with water and extracted three times with DCM. The combined organic layers were washed with brine, dried over Na₂SO₄, filtered, and concentrated under reduced pressure to afford methyl 3-[6-amino-2-[(2R,6S)-2-methyl-6-(1-methyl-6-oxo-3-pyridyl) morpholin-4-yl]-5-nitro-pyrimidin-4-yl]bicyclo[1.1.1]pentane-1-carboxylate (Int-C49, 451 mg, 0.96 mmol, 99% yield), which was used without further purification. ESI-MS: [M+H]⁺=471.4.

An analogous procedure was followed to obtain the following intermediates.

| Structure | Starting Material | Characterization |
|---|---|---|
| 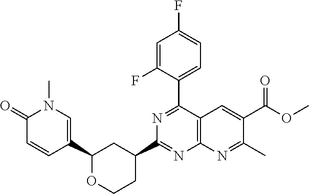 [SMILES: COC(=O)c1cc2c(-c3ccc(F)cc3F)nc([C@H]3CCO[C@@H](c4ccc(=O)n(C)c4)C3)nc2nc1C] Int-E19 | 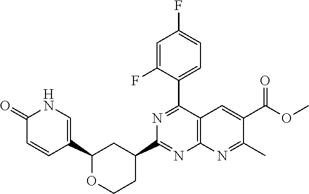 Int-E18 | The crude material was purified by flash chromatography using a 0-20% MeOH in DCM elution gradient to yield methyl 4-(2,4- difluorophenyl)-7-methyl-2- ((2R,4S)-2-(1-methyl-6-oxo-1,6- dihydropyridin-3-yl)tetrahydro- 2H-pyran-4-yl)pyrido[2,3- d]pyrimidine-6-carboxylate (Int- E19, 35 mg, 0.07 mmol, 50% yield) as a solid. ESI-MS: [M + H]⁺ = 507.3. |
| 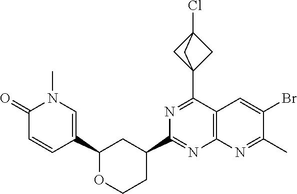 [SMILES: Cc1nc2nc([C@H]3CCO[C@@H](c4ccc(=O)n(C)c4)C3)nc(C34CC(Cl)(C3)C4)c2cc1Br] Int-E32 | 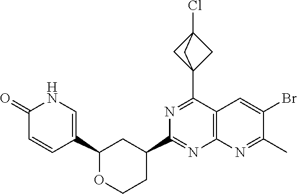 Int-E31 | The crude reaction mixture was purified by flash column chromatography (40 g SiO₂ column) using an elution gradient of 1-10% MeOH in DCM to yield 5-[(2R,4S)-4-[6-bromo-7- methyl-4-[3-(trifluoromethyl)- 1-bicyclo[1.1.1]pentanyl] pyrido[2,3- d]pyrimidin-2- yl]tetrahydropyran-2-yl]-1- methyl-pyridin-2-one (Int-E32, 164 mg, 0.30 mmol, 78% yield) as a foam. ESI-MS: [M + H]⁺ = 549.1. |

### Method Int 91. Intermediate Int-C50:3-[6-amino-2-[(2R,6S)-2-methyl-6-(1-methyl-6-oxo-3-pyridyl) morpholin-4-yl]-5-nitro-pyrimidin-4-yl]bicyclo[1.1.1]pentane-1-carboxamide

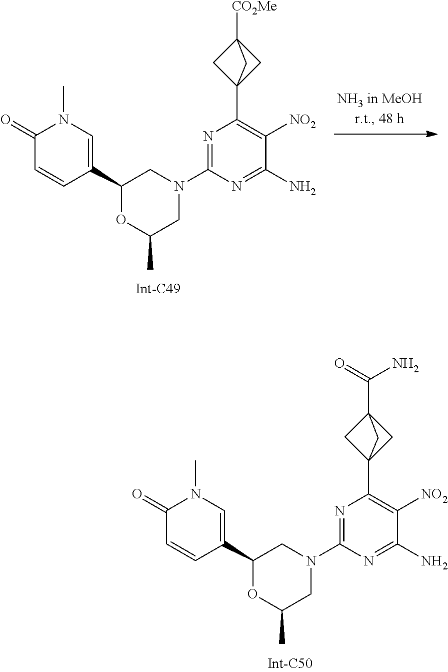 [SMILES: COC(=O)C12CC(c3nc(N4C[C@@H](C)O[C@@H](c5ccc(=O)n(C)c5)C4)nc(N)c3[N+](=O)[O-])(C1)C2 ; C[C@@H]1CN(c2nc(N)c([N+](=O)[O-])c(C34CC(C(N)=O)(C3)C4)n2)C[C@H](c2ccc(=O)n(C)c2)O1]

Methyl 3-[6-amino-2-[(2R,6S)-2-methyl-6-(1-methyl-6-oxo-3-pyridyl) morpholin-4-yl]-5-nitro-pyrimidin-4-yl]bicyclo[1.1.1]pentane-1-carboxylate (Int-C49, 1 equiv., 465 mg, 0.99 mmol) was stirred in a solution of 7.0 M ammonia (28 equiv., 4.0 mL, 27.7 mmol) in methanol for 2 days. The reaction was concentrated under reduced pressure to afford 3-[6-amino-2-[(2R,6S)-2-methyl-6-(1-methyl-6-oxo-3-pyridyl) morpholin-4-yl]-5-nitro-pyrimidin-4-yl]bicyclo[1.1.1]pentane-1-carboxamide (Int-C50, 445 mg, 0.98 mmol, 99% yield) which was used without further purification. ESI-MS: [M+H]⁺=456.3.

### Method Int 92. Intermediate Int-C51:3-[6-amino-2-[(2R,6S)-2-methyl-6-(1-methyl-6-oxo-3-pyridyl) morpholin-4-yl]-5-nitro-pyrimidin-4-yl]bicyclo[1.1.1]pentane-1-carboxamide

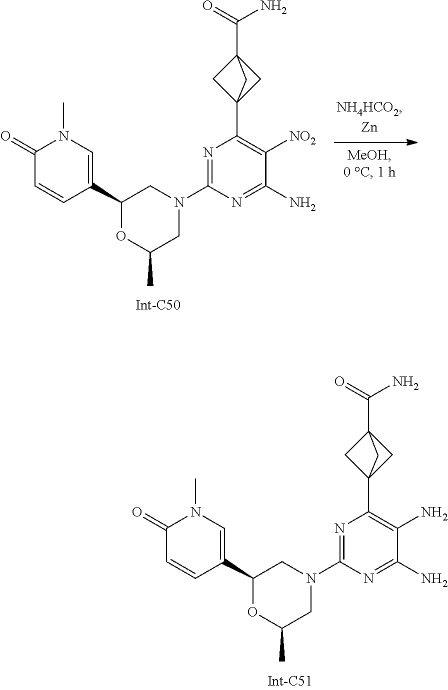 [SMILES: C[C@@H]1CN(c2nc(N)c([N+](=O)[O-])c(C34CC(C(N)=O)(C3)C4)n2)C[C@H](c2ccc(=O)n(C)c2)O1 ; C[C@@H]1CN(c2nc(N)c(N)c(C34CC(C(N)=O)(C3)C4)n2)C[C@H](c2ccc(=O)n(C)c2)O1]

A mixture of 3-[6-amino-2-[(2R,6S)-2-methyl-6-(1-methyl-6-oxo-3-pyridyl) morpholin-4-yl]-5-nitro-pyrimidin-4-yl]bicyclo[1.1.1]pentane-1-carboxamide (Int-C50, 1 equiv., 445 mg, 0.98 mmol), ammonium formate (8 equiv., 493 mg, 7.82 mmol) and zinc (8 equiv., 511 mg, 7.82 mmol) was stirred at 0° C. for 1 hour. The reaction mixture was filtered through a Celite pad and concentrated under reduced pressure to afford 3-[5,6-diamino-2-[(2R,6S)-2-methyl-6-(1-methyl-6-oxo-3-pyridyl) morpholin-4-yl]pyrimidin-4-yl]bicyclo[1.1.1]pentane-1-carboxamide (Int-C51, 415 mg, 0.98 mmol, 99% yield), which was used without further purification. ESI-MS: [M+H]⁺=426.4.

### Method Int 93. Intermediate Int-D1: Methyl(2R,4S)-2-(6-benzyloxy-3-pyridyl)tetrahydropyran-4-carboxylate

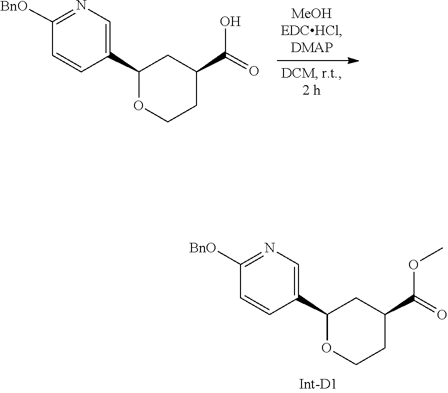 [SMILES: O=C(O)[C@H]1CCO[C@@H](c2ccc(OCc3ccccc3)nc2)C1 ; COC(=O)[C@H]1CCO[C@@H](c2ccc(OCc3ccccc3)nc2)C1]

A solution of (2R,4S)-2-(6-benzyloxy-3-pyridyl)tetrahydropyran-4-carboxylic acid (1 equiv., 4.0 g, 12.8 mmol), MeOH (2 equiv., 1.0 mL, 25.5 mmol) and DMAP (0.2 equiv., 312 mg, 2.6 mmol) in DCM (35 mL) was stirred at r.t. for 2 h. The reaction mixture was quenched with 25 mL of citric acid 10% (aq.), then extracted with DCM (3×50 mL). The combined organic phases were washed with water and brine, dried over anhydrous Na₂SO₄, filtered and concentrated under reduced pressure to yield methyl(2R,4S)-2-(6-benzyloxy-3-pyridyl)tetrahydropyran-4-carboxylate (Int-D1, 4.1 g, 12.4 mmol, 96% yield) as a solid. ESI-MS: 328.3. ¹H NMR (CDCl₃, 400 MHz): δ 8.11 (d, J=2.3 Hz, 1H), 7.61 (dd, J=8.6, 2.4 Hz, 1H), 7.44 (d, J=7.1 Hz, 2H), 7.37-7.27 (m, 3H), 6.78 (d, J=8.5 Hz, 1H), 5.36 (s, 2H), 4.31 (dd, J=11.4, 2.1 Hz, 1H), 4.18 (ddd, J=11.6, 4.5, 1.5 Hz, 1H), 4.11 (q, J=7.2 Hz, 1H), 3.69 (s, 3H), 3.60 (td, J=12.0, 2.5 Hz, 1H), 2.70 (qd, J=8.1, 4.0 Hz, 1H), 2.10-2.05 (m, 1H), 1.92-1.67 (m, 3H), 1.25 (t, J=7.1 Hz, 1H).

### Method Int 94. Intermediate Int-D2: Methyl(2R,4S)-2-(1-amino-6-oxo-3-pyridyl)tetrahydropyran-4-carboxylate

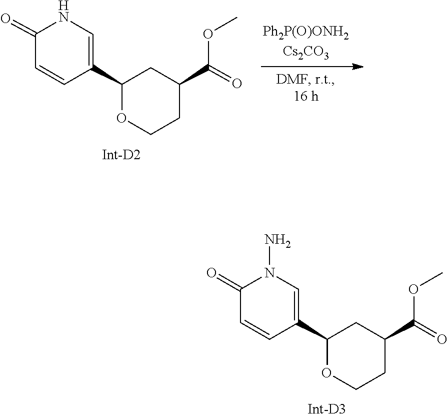 [SMILES: COC(=O)[C@H]1CCO[C@@H](c2ccc(=O)[nH]c2)C1 ; COC(=O)[C@H]1CCO[C@@H](c2ccc(=O)n(N)c2)C1]

A 250 mL round-bottomed flask equipped with a Teflon-coated stirring bar was charged with methyl(2R,4S)-2-(6-oxo-1H-pyridin-3-yl)tetrahydropyran-4-carboxylate (Int-D2, 1 equiv., 6.6 g, 27.9 mmol) then cooled down to 0° C. DMF (139 mL) was added followed by cesium carbonate (3 equiv., 27.3 g, 83.7 mmol) and o-diphenylphosphorylhydroxylamine (2 equiv., 13.0 g, 55.8 mmol). The reaction mixture was stirred at r.t. under argon for 16 h, concentrated under reduced pressure, reconstituted in DCM, and filtered over a Celite pad, rinsing with 350 mL of DCM. The filtrate was concentrated under reduced pressure to approximately 40 mL volume. Sat. NaHCO₃ (aq.)(50 mL) was added, the layers were separated and the aqueous phase was extracted with DCM (5×130 mL). The organic phases were combined, washed with brine (70 mL), dried over anhydrous Na₂SO₄, filtered, and concentrated under reduced pressure to yield methyl(2R,4S)-2-(1-amino-6-oxo-3-pyridyl)tetrahydropyran-4-carboxylate (Int-D3, 6.6 g, 24.7 mmol, 88% yield) as a solid. ¹H NMR (CDCl₃, 400 MHz): δ 7.61 (d, J=2.5 Hz, 1H), 7.35 (dd, J=9.4, 2.5 Hz, 1H), 6.61 (d, J=9.4 Hz, 1H), 5.15 (s, 2H), 4.18-4.09 (m, 2H), 3.70 (s, 3H), 3.57 (td, J=12.1, 2.2 Hz, 1H), 2.67 (tt, J=12.2, 3.8 Hz, 1H), 2.07 (dt, J=13.3, 1.8 Hz, 1H), 1.92-1.88 (m, 1H), 1.76 (ddd, J=25.8, 12.5, 4.6 Hz, 1H), 1.64 (dd, J=25.2, 12.1 Hz, 1H). ESI-MS: [M+H]⁺=253.2.

An analogous procedure was followed to obtain the following intermediates.

| Structure | Starting Material | Characterization |
|---|---|---|
| 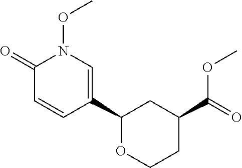 | 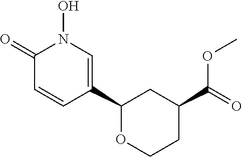 | The procedure afforded 1-amino-5-[(2R,4S)-4- [4-(4,4-difluorocyclohexyl)-6,7-dimethyl- pteridin-2-yl]tetrahydropyran-2-yl]pyridin-2-one (Int-D43, 500 mg, 1.06 mmol, 97% yield) as a gum. LCMS: [M + H]⁺ = 471.2. |
| Int-D43 | Example 224 | |

### Method Int 95. Intermediate Int-D4: Methyl(2R,4S)-2-[1-(diallylamino)-6-oxo-3-pyridyl]tetrahydropyran-4-carboxylate

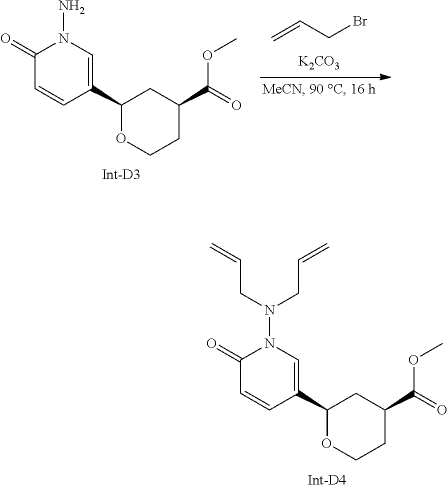

A reaction vial was charged with methyl(2R,4S)-2-(1-amino-6-oxo-3-pyridyl)tetrahydropyran-4-carboxylate (Int-D3, 1 equiv., 2.8 g, 11.1 mmol), MeCN (40 mL) and allyl bromide (3 equiv., 2.9 mL, 33.4 mmol). The vial was sealed, purged with argon, and heated at 90° C. for 18 h. Another portion of allyl bromide (2 equiv., 2.0 mL, 23.0 mmol) was added, and the vial stirred at 90° C. for a further 5 hours. The reaction mixture was filtered through a Celite pad, rinsing with MeCN, and the filtrate was concentrated under reduced pressure. The resulting solid was purified by flash chromatography (120 g SiO₂ column) using an elution gradient of 25-80% EtOAc in hexanes to yield methyl(2R,4S)-2-[1-(diallylamino)-6-oxo-3-pyridyl]tetrahydropyran-4-carboxylate (Int-D4, 1.98 g, 5.9 mmol, 53% yield) as a solid. ESI-MS: [M+H]⁺=333.4. ¹H NMR (CDCl₃, 400 MHz): δ 7.27 (d, J=2.5 Hz, 1H), 6.48-6.46 (m, 1H), 5.77 (dd, J=17.0, 10.2 Hz, 2H), 5.18 (dd, J=17.2, 1.4 Hz, 2H), 5.05 (d, J=9.8 Hz, 2H), 4.16-4.10 (m, 3H), 4.04 (dd, J=11.4, 1.8 Hz, 1H), 3.77-3.73 (m, 2H), 3.70 (s, 3H), 3.55 (td, J=12.0, 2.3 Hz, 1H), 2.65 (tt, J=12.3, 3.9 Hz, 1H), 2.01 (dt, J=13.3, 1.7 Hz, 1H), 1.87-1.77 (m, 2H), 1.62 (t, J=12.5 Hz, 1H).

### Method Int 96. Intermediate Int-D5: Methyl(2R,4S)-2-[1-(2,5-dihydropyrrol-1-yl)-6-oxo-3-pyridyl]tetrahydropyran-4-carboxylate

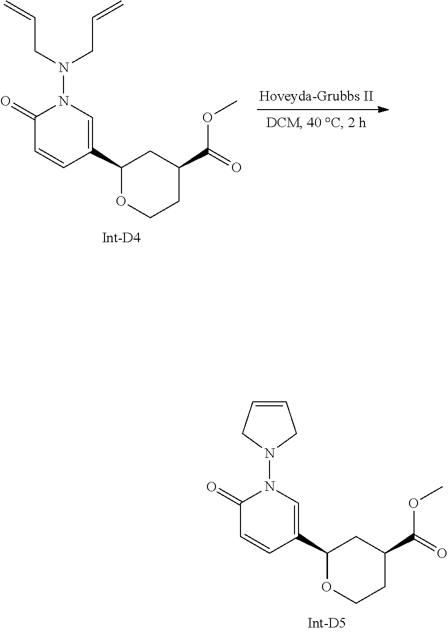 [SMILES: C=CCN(CC=C)n1cc([C@H]2C[C@@H](C(=O)OC)CCO2)ccc1=O ; COC(=O)[C@H]1CCO[C@@H](c2ccc(=O)n(N3CC=CC3)c2)C1]

Hoveyda-Grubbs 2nd Generation catalyst (0.02 equiv., 15 mg, 0.0235 mmol) was added to a stirring solution of methyl(2R,4S)-2-[1-(diallylamino)-6-oxo-3-pyridyl]tetrahydropyran-4-carboxylate (Int-D4, 1 equiv., 665 mg, 2.0 mmol) in DCM (20 mL) at 40° C., and the resulting green reaction mixture was stirred at 40° C. for 1 h. The reaction mixture was treated with 150 mg of silica gel, stirred for 30 min at r.t., and filtered through a Celite pad, rinsing with DCM. The filtrate was concentrated under reduced pressure onto Celite (9 g) and purified by flash column chromatography (80 g SiO₂ column) using an elution gradient of 1-10% MeOH in DCM to yield methyl(2R,4S)-2-[1-(2,5-dihydropyrrol-1-yl)-6-oxo-3-pyridyl]tetrahydropyran-4-carboxylate (Int-D5, 483 mg, 1.6 mmol, 79% yield) as a solid. ESI-MS: [M+H]⁺=305.2. ¹H NMR (CDCl₃, 400 MHz): δ 7.53 (d, J=2.5 Hz, 1H), 7.33 (dd, J=9.5, 2.6 Hz, 1H), 6.58 (d, J=9.6 Hz, 1H), 5.82 (s, 2H), 4.18 (s, 4H), 4.09 (dd, J=11.5, 2.4 Hz, 1H), 3.74 (d, J=6.6 Hz, 1H), 3.70 (s, 3H), 3.60-3.54 (m, 1H), 2.71-2.63 (m, 1H), 2.04-2.12 (1H), 1.88-1.67 (m, 3H).

### Method Int 97. Intermediate Int-D7: (2R,4S)-2-(6-oxo-1-pyrrolidin-1-yl-3-pyridyl)tetrahydropyran-4-carboxylic acid

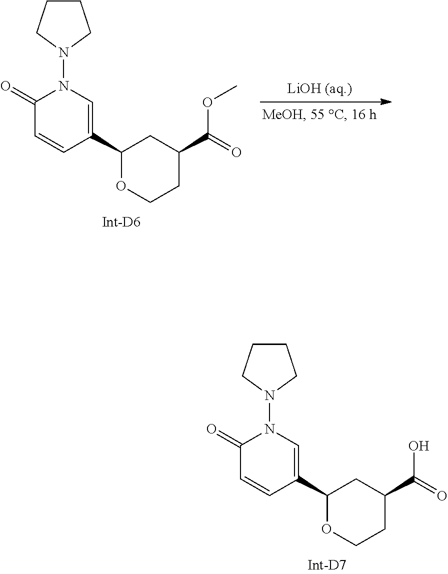 [SMILES: COC(=O)[C@H]1CCO[C@@H](c2ccc(=O)n(N3CCCC3)c2)C1 ; O=C(O)[C@H]1CCO[C@@H](c2ccc(=O)n(N3CCCC3)c2)C1]

A solution of lithium hydroxide (2.1 equiv., 40 mg, 1.7 mmol) in water (0.5 mL) was added to a solution of methyl(2R,4S)-2-(6-oxo-1-pyrrolidin-1-yl-3-pyridyl)tetrahydropyran-4-carboxylate (Int-D6, 1 equiv., 240 mg, 0.78 mmol) in MeOH (2 mL). The reaction mixture was stirred at 55° C. for 16 h, then dissolved in water, acidified until pH=2 with a IN HCl (aq.), and extracted four times with EtOAc. The combined organic phases were dried over anhydrous Na₂SO₄, filtered, and concentrated under reduced pressure to yield (2R,4S)-2-(6-oxo-1-pyrrolidin-1-yl-3-pyridyl)tetrahydropyran-4-carboxylic acid (Int-D6, 186 mg, 0.64 mmol, 81% yield) as a solid. ESI-MS: [M+H]⁺=293.3. ¹H NMR (400 MHz, DMSO-d₆): δ 11.83-12.36 (1H), 7.53 (d, J=2.5 Hz, 1H), 7.39 (dd, J=9.4, 2.5 Hz, 1H), 6.37 (d, J=9.4 Hz, 1H), 4.11 (d, J=9.8 Hz, 1H), 3.96 (dd, J=11.0, 3.9 Hz, 1H), 3.47-3.42 (m, 1H), 3.26 (d, J=8.2 Hz, −3H), 2.64-2.55 (m, 1H), 1.95-1.73 (m, 5H), 1.51-1.37 (m, 2H).

An analogous procedure was followed to obtain the following intermediates.

| Structure | Starting Material | Characterization |
|---|---|---|
| 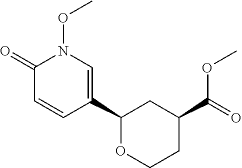 | 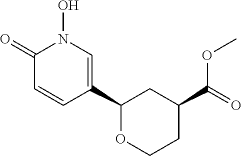 | Modifications: The reaction was run with 5 equiv. LiOH at 40 °C for 1 h to afford (2R,4S)- 2-[1-(6,6-difluoro-3-azabicyclo[3.1.0]hexan-3- yl)-6-oxo-3-pyridyl\|tetrahydropyran-4- carboxylic acid (Int-C43, 44 mg, 0.13 mmol, 99% yield) as a solid. ESI-MS: [M + H]⁺ = 341.2. |
| Int-C43 | Int-C42 | |

### Method Int 98. Intermediate Int-D9: methyl(2R,4S)-2-[1-(6,6-difluoro-3-azabicyclo[3.1.0]hexan-3-yl)-6-oxo-3-pyridyl]tetrahydropyran-4-carboxylate

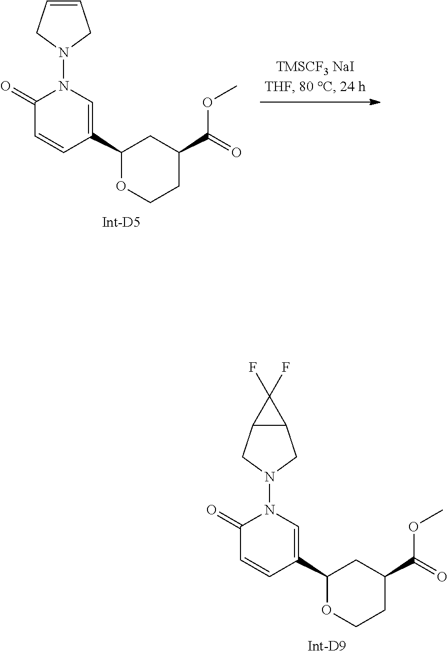 [SMILES: COC(=O)[C@H]1CCO[C@@H](c2ccc(=O)n(N3CC=CC3)c2)C1 ; COC(=O)[C@H]1CCO[C@@H](c2ccc(=O)n(N3CC4C(C3)C4(F)F)c2)C1]

Sodium iodide (0.4 equiv., 39 mg, 0.26 mmol) was added to a solution of methyl(2R,4S)-2-[1-(2,5-dihydropyrrol-1-yl)-6-oxo-3-pyridyl]tetrahydropyran-4-carboxylate (Int-D5, 1 equiv, 200 mg, 0.66 mmol) in anhydrous THF (5 mL) under a nitrogen atmosphere. The reaction mixture was refluxed, and five portions of trimethyl(trifluoromethyl) silane (3.5 equiv, 340 μL, 2.3 mmol) were added over 24 h. The reaction mixture was concentrated under reduced pressure and purified by flash chromatography (24 g SiO₂ column) using an elution gradient of 10-60% EtOAc:EtOH (3:1) in hexanes to yield methyl (2R,4S)-2-[1-(6,6-difluoro-3-azabicyclo[3.1.0]hexan-3-yl)-6-oxo-3-pyridyl]tetrahydropyran-4-carboxylate (Int-D9, 105 mg, 0.15 mmol, 22% yield) as a solid. ESI-MS: [M+H]⁺=355.3.

### Method Int 99. Intermediate Int-D12:6-chloro-2-methyl-1H-pyrimido[5,4-d]pyrimidin-4-one hydrochloride

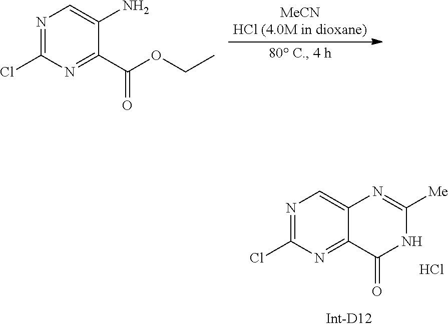

A vial was charged with a solution of ethyl 5-amino-2-chloro-pyrimidine-4-carboxylate (1 equiv., 2.0 g, 9.9 mmol) in HCl (4 M solution in 1,4-dioxane, 6.1 equiv., 15 mL, 60.0 mmol) and MeCN (6 equiv., 3.1 mL, 59.4 mmol). The reaction mixture was sealed, stirred at 80° C. for 4 h, cooled to r.t., and filtered. The solids were rinsed with MeCN and dried under high vacuum to obtain the hydrochloride salt of 6-chloro-2-methyl-1H-pyrimido[5,4-d]pyrimidin-4-one (Int-D12, 2.35 g, 10.1 mmol, 100% yield) as a solid. ESI-MS: [M+H]⁺=197.0.

### Method Int 100. Intermediate Int-D14:6-chloro-2,3-dimethyl-8-[3-(trifluoromethyl)-1-bicyclo[1.1.1]pentanyl]pyrimido[5,4-d]pyrimidin-4-one

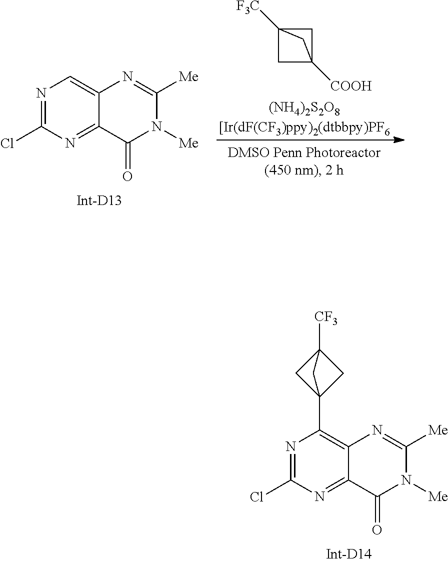

A reaction vial equipped with a Teflon-coated magnetic stirring bar was charged with Ir[dF(CF₃)ppy]₂ (dtbbpy)PF₆ (0.005 equiv., 28 mg, 0.026 mmol), ammonium persulfate (2 equiv., 2.32 g, 10.2 mmol), 6-chloro-2,3-dimethyl-pyrimido[5,4-d]pyrimidin-4-one (Int-D13, 1 equiv., 1.07 g, 5.1 mmol) and 3-(trifluoromethyl) bicyclo[1.1.1]pentane-1-carboxylic acid (5 equiv., 4.58 g, 25.4 mmol). The vial was sealed, purged three times with argon, and anhydrous DMSO (28 mL) was added. The reaction was placed in a Penn PhD photoreactor M2 and irradiated at 450 nm for 2 h. Then the reaction mixture was diluted with DCM, washed with sat. NaHCO₃ (aq.), and the aqueous phase was extracted twice with DCM. The combined organic phases were washed with brine, dried over anhydrous Na₂SO₄, filtered, and concentrated under reduced pressure. The resulting residue was purified by flash chromatography (40 g SiO₂ column) using an elution gradient of 2-65% EtOAc:EtOH (3:1) in hexanes to yield 6-chloro-2,3-dimethyl-8-[3-(trifluoromethyl)-1-bicyclo[1.1.1]pentanyl]pyrimido[5,4-d]pyrimidin-4-one (Int-D14, 373 mg, 1

### Method Int 101. Intermediate Int-D18:2,4,6-trichloro-5-(1,3-dioxolan-2-yl)pyrimidine

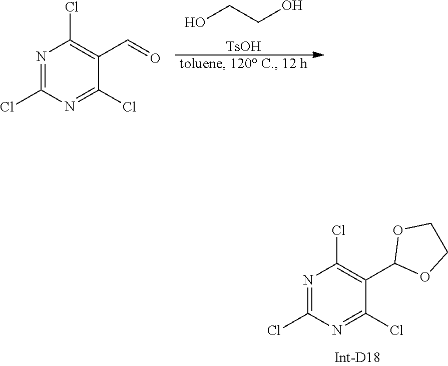

To a solution of 2,4,6-trichloropyrimidine-5-carbaldehyde (1 equiv., 25.0 g, 118 mmol) and ethane-1,2-diol (3 equiv., 20 mL, 355 mmol) in toluene (250 mL) was added TsOH (0.2 equiv., 4.07 g, 23.6 mmol). The mixture was stirred at 120° C. for 12 h, concentrated under reduced pressure, and purified by silica gel column (PE/EtOAc=5:1, R_{f}=0.4) to give 2,4,6-trichloro-5-(1,3-dioxolan-2-yl)pyrimidine (19.00 g, 74.4 mmol, 63% yield) as an oil. LCMS: [M+H]⁺=254.9.

¹H NMR (400 MHz, CDCl₃) δ 4.04-4.16 (m, 2H) 4.27-4.36 (m, 2H) 6.32 (s, 1H).

### Method Int 102. Intermediate Int-D-19:2,4-dichloro-5-(1,3-dioxolan-2-yl)-6-(1-ethoxyvinyl)pyrimidine

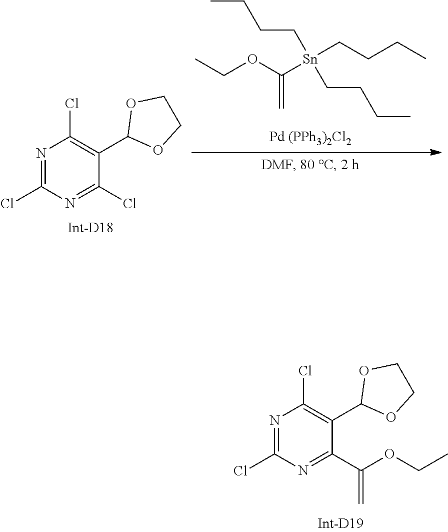 [SMILES: Clc1nc(Cl)c(C2OCCO2)c(Cl)n1 ; C=[C](OCC)[Sn]([CH2]CCC)([CH2]CCC)[CH2]CCC ; C=C(OCC)c1nc(Cl)nc(Cl)c1C1OCCO1]

To a mixture of 2,4,6-trichloro-5-(1,3-dioxolan-2-yl)pyrimidine (Int-D18, 1 equiv., 19.0 g, 74.4 mmol) in DMF (200 mL) were added tributyl(1-ethoxyvinyl) stannane (1 equiv., 25 mL, 74.4 mmol) and Pd(PPh₃)₂Cl₂ (0.1 equiv., 5220 mg, 7.44 mmol). The reaction flask was degassed and refilled with N₂, repeatedly for three cycles. The mixture was stirred at 80° C. for 2 h. The mixture was cooled to 25° C., added into sat. KF (aq., 800 mL) and stirred for 2 h at 25° C. The mixture was extracted with ethyl acetate (500 mL×3), washed with saturated brine solution (500 mL), and dried (Na₂SO₄) before concentration to dryness. The residue was purified by silica gel column (PE/EtOAc=5:1, R_{f}=0.4) to give 2,4-dichloro-5-(1,3-dioxolan-2-yl)-6-(1-ethoxyvinyl)pyrimidine (Int-D19, 16.0 g, 45.4 mmol, 61% yield) as an oil. LCMS: [M+H]⁺=291.1. ¹H NMR (400 MHz, CDCl₃) δ 1.38 (t, J=7.0 Hz, 3H) 3.88-3.98 (m, 2H) 3.99-4.08 (m, 2H) 4.24-4.33 (m, 2H) 4.59 (d, J=3.1 Hz, 1H) 4.67-4.70 (m, 1H) 6.11 (s, 1H).

### Method Int 103. Intermediate Int-D20: ethyl 2,6-dichloro-5-(1,3-dioxolan-2-yl)pyrimidine-4-carboxylate

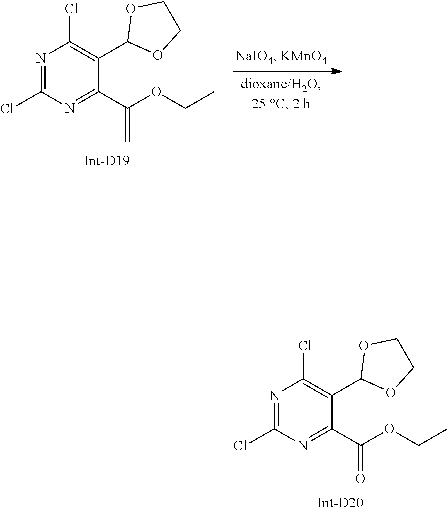 [SMILES: C=C(OCC)c1nc(Cl)nc(Cl)c1C1OCCO1 ; CCOC(=O)c1nc(Cl)nc(Cl)c1C1OCCO1]

To a solution of sodium metaperiodate (2 equiv., 15.78 g, 73.8 mmol) in water (75 mL) was added a solution of 2,4-dichloro-5-(1,3-dioxolan-2-yl)-6-(1-ethoxyvinyl)pyrimidine (Int-D19, 1 equiv., 13.0 g, 36.9 mmol) in 1,4-dioxane (150 mL). Then potassium permanganate (0.4 equiv., 2.33 g, 14.8 mmol) was added to the mixture slowly at 40-50° C. The mixture was stirred at 25° C. for 2 hand then added into water (500 mL) and extracted with EtOAc (300 mL×3). The organic phase was concentrated under vacuum to give a crude. The crude was purified by silica gel column (PE/EtOAc=5:1, R_{f}=0.4) to give ethyl 2,6-dichloro-5-(1,3-dioxolan-2-yl)pyrimidine-4-carboxylate (Int-D20, 6.00 g, 20.5 mmol, 56% yield) as an oil. LCMS: [M+H]⁺=292.9. ¹H NMR (400 MHz, CDCl₃) δ 1.38-1.43 (m, 3H) 4.00-4.07 (m, 4H) 4.41 (q, J=7.2 Hz, 2H) 6.16 (s, 1H).

### Method Int 104. Intermediate Int-D21: ethyl 2-chloro-5-(1,3-dioxolan-2-yl)-6-[2-fluoro-4-(trifluoromethyl)phenyl]pyrimidine-4-carboxylate

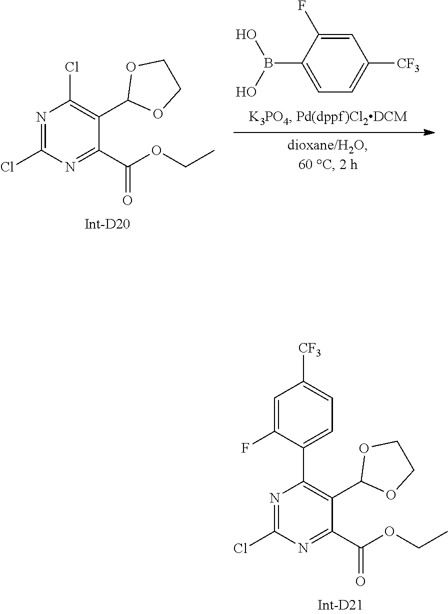 [SMILES: CCOC(=O)c1nc(Cl)nc(Cl)c1C1OCCO1 ; OB(O)c1ccc(C(F)(F)F)cc1F ; CCOC(=O)c1nc(Cl)nc(-c2ccc(C(F)(F)F)cc2F)c1C1OCCO1]

To a solution of ethyl 2,6-dichloro-5-(1,3-dioxolan-2-yl)pyrimidine-4-carboxylate (Int-D20, 1 equiv., 700 mg, 2.39 mmol) and [2-fluoro-4-(trifluoromethyl)phenyl]boronic acid (0.95 equiv., 472 mg, 2.27 mmol) in 1,4-dioxane (7 mL) and water (0.70 mL) were added K₃PO₄ (2 equiv., 1013 mg, 4.78 mmol) and Pd(dppf)Cl₂·DCM (0.1 equiv., 175 mg, 0.239 mmol) under N₂. The mixture was stirred for 2 hours at 60° C., added into water (60 mL), and then extracted with ethyl acetate (30 mL×3). The organic phase was washed with saturated brine solution (30 mL). The organics were then separated and dried (Na₂SO₄) before concentration to dryness. The crude was then purified by prep-HPLC (~60% ACN in 0.1% FA (aq.)) to give ethyl 2-chloro-5-(1,3-dioxolan-2-yl)-6-[2-fluoro-4-(trifluoromethyl)phenyl]pyrimidine-4-carboxylate (620 mg, 1.47 mmol, 62% yield) as a solid. LCMS: [M+H]⁺=420.9. ¹H NMR (400 MHz, CDCl₃) δ 1.44 (t, J=7.2 Hz, 3H) 3.79-3.90 (m, 4H) 4.47 (q, J=7.1 Hz, 2H) 5.97 (s, 1H) 7.47 (d, J=9.4 Hz, 1H) 7.53-7.59 (m, 1H) 7.63-7.70 (m, 1H).

An analogous procedure was followed to obtain the following intermediates.

| Structure | Starting Material | Characterization |
|---|---|---|
| 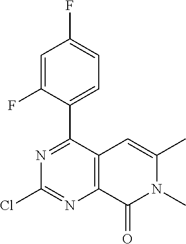 [SMILES: Cc1cc2c(-c3ccc(F)cc3F)nc(Cl)nc2c(=O)n1C] Int-D33 | 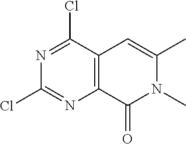 D-32 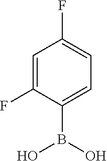 [SMILES: OB(O)c1ccc(F)cc1F] | Modifications: The reaction was run with (2,4- difluorophenyl)boronic acid at 40 °C for 1 h. The reaction crude was purified by column chromatography (gradient elution of hexane/EtOAc = 1:1~0:1) to give 2- chloro-4-(2,4-difluorophenyl)-6,7-dimethyl-pyrido[3,4- d]pyrimidin-8-one (Int-D33, 330 mg, 1.03 mmol, 83% yield) as a solid. LCMS: [M + H]⁺ = 322.0. ¹H NMR (400 MHz, DMSO-d₆) δ 7.71 (q, J = 7.8 Hz, 1H), 7.61- 7.51 (m, 1H), 7.36 (br t, J = 7.9 Hz, 1H), 6.27 (br s, 1H), 3.56 (s, 3H), 2.43 (s, 3H) |
| 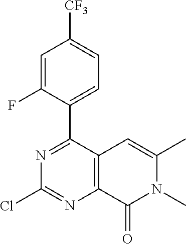 [SMILES: Cc1cc2c(-c3ccc(C(F)(F)F)cc3F)nc(Cl)nc2c(=O)n1C] Int-D36 | 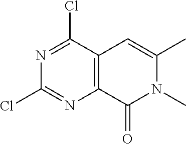 Int-D32 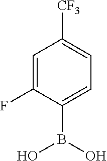 [SMILES: OB(O)c1ccc(C(F)(F)F)cc1F] | Modifications: The reaction was run with [2-fluoro-4- (trifluoromethyl)phenyl]boronic acid at 40 °C for 1 h. The reaction crude was purified by column chromatography (gradient elution of hexane/EtOAc = 1:0 to 0:1) to give 2-chloro-4-[2-fluoro-4- (trifluoromethyl)phenyl]-6,7-dimethyl-pyrido[3,4- d]pyrimidin-8-one (Int-D36, 650 mg, 1.75 mmol, 66% yield) as a solid. LCMS: [M + H]⁺ = 372.0. ¹H NMR (400 MHz, DMSO-d₆) δ 7.99 (d, J = 9.9 Hz, 1H), 7.91- 7.82 (m, 2H), 6.30 (d, J = 2.1 Hz, 1H), 3.56 (s, 3H), 2.43 (s, 3H). |
| 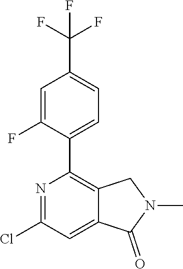 [SMILES: CN1Cc2c(cc(Cl)nc2-c2ccc(C(F)(F)F)cc2F)C1=O] Int-E45 | 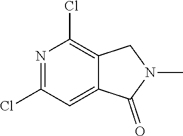 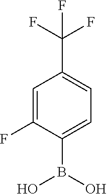 [SMILES: OB(O)c1ccc(C(F)(F)F)cc1F] | Modifications: The reaction was run with 2 equiv. of K2CO3 instead of K₃PO₄, and purified by reverse-phase HPLC to afford 6-chloro-4-(2-fluoro-4- (trifluoromethyl)phenyl)-2-methyl-2,3-dihydro-1H- pyrrolo[3,4-c]pyridin-1-one (Int-E45, 383 mg, 1.11 mmol, 48% yield) as a solid. ESI-MS: [M + H]⁺ = 345.0. ¹H NMR (400 MHz, CDCl₃) δ 7.83 (br s, 2H), 7.63- 7.46 (m, 2H), 4.57-4.37 (m, 2H), 3.23 (s, 3H) |

### Method Int 105. Intermediate Int-D22: ethyl 2-[(2S,6R)-2-(6-benzyloxy-3-pyridyl)-6-methyl-morpholin-4-yl]-5-(1,3-dioxolan-2-yl)-6-[2-fluoro-4-(trifluoromethyl)phenyl]pyrimidine-4-carboxylate

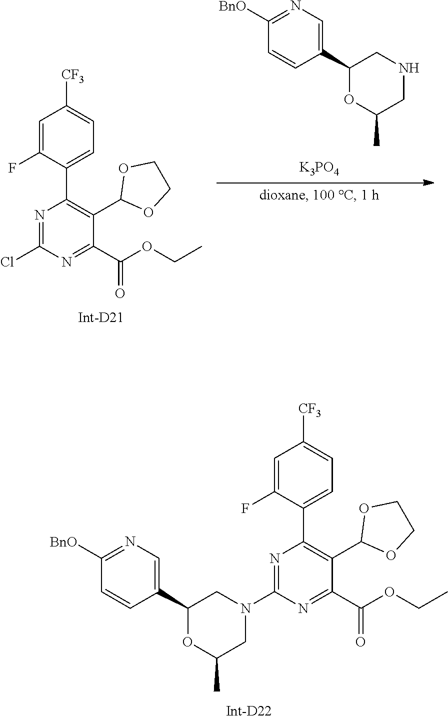 [SMILES: CCOC(=O)c1nc(Cl)nc(-c2ccc(C(F)(F)F)cc2F)c1C1OCCO1 ; C[C@@H]1CNC[C@H](c2ccc(OCc3ccccc3)nc2)O1 ; CCOC(=O)c1nc(N2C[C@@H](C)O[C@@H](c3ccc(OCc4ccccc4)nc3)C2)nc(-c2ccc(C(F)(F)F)cc2F)c1C1OCCO1]

To a solution of ethyl 2-chloro-5-(1,3-dioxolan-2-yl)-6-[2-fluoro-4-(trifluoromethyl)phenyl]pyrimidine-4-carboxylate (Int-D21, 1 equiv., 250 mg, 0.594 mmol) and (2S,6R)-2-(6-benzyloxy-3-pyridyl)-6-methyl-morpholine (1.1 equiv., 186 mg, 0.654 mmol) in 1,4-dioxane (5 mL) was added K₃PO₄ (2 equiv., 252 mg, 1.19 mmol), and the mixture was stirred for 1 hour at 100° C. The reaction solution was added into water (30 mL) and then extracted with ethyl acetate (10 mL×2). The organics was washed with 10 mL saturated brine solution, separated, dried over Na₂SO₄, and concentrated under reduced pressure to dryness. The crude was then purified by silica gel column (PE/EtOAc=3:1, R_{f}=0.6) to give ethyl 2-[(2S,6R)-2-(6-benzyloxy-3-pyridyl)-6-methyl-morpholin-4-yl]-5-(1,3-dioxolan-2-yl)-6-[2-fluoro-4-(trifluoromethyl)phenyl]pyrimidine-4-carboxylate (Int-D22, 360 mg, 0.538 mmol, 91% yield) as a solid. LCMS [M+H]⁺=669.2. ¹H NMR (400 MHz, CDCl₃) δ 1.31 (br d, J=6.1 Hz, 3H) 1.42 (t, J=7.1 Hz, 3H) 2.70-2.81 (m, 1H) 2.90 (br t, J=12.2 Hz, 1H) 3.82 (s, 4H) 4.42 (q, J=7.1 Hz, 2H) 4.53 (br d, J=9.5 Hz, 1H) 4.66-4.83 (m, 2H) 5.39 (s, 2H) 5.68 (s, 1H) 6.82 (d, J=8.6 Hz, 1H) 7.29-7.34 (m, 1H) 7.35-7.47 (m, 5H) 7.48-7.53 (m, 1H) 7.55-7.61 (m, 1H) 7.66-7.73 (m, 1H) 8.20 (s, 1H).

### Method Int 106. Intermediate Int-D23: ethyl 2-[(2S,6R)-2-(6-benzyloxy-3-pyridyl)-6-methyl-morpholin-4-yl]-6-[2-fluoro-4-(trifluoromethyl)phenyl]-5-formyl-pyrimidine-4-carboxylate

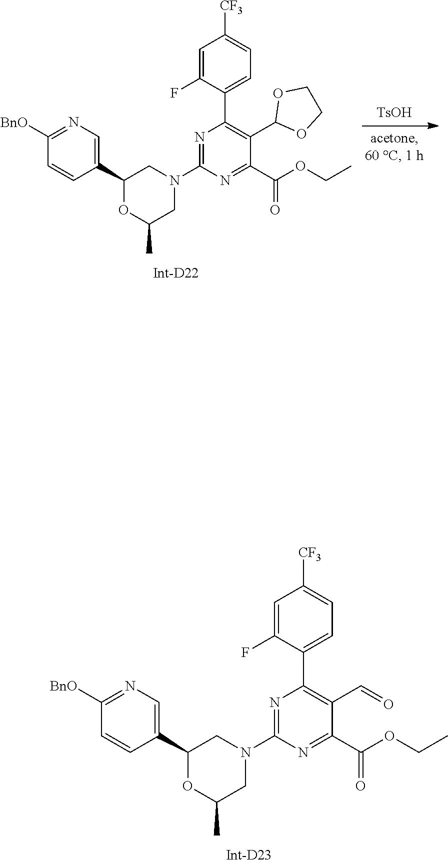 [SMILES: CCOC(=O)c1nc(N2C[C@@H](C)O[C@@H](c3ccc(OCc4ccccc4)nc3)C2)nc(-c2ccc(C(F)(F)F)cc2F)c1C1OCCO1 ; CCOC(=O)c1nc(N2C[C@@H](C)O[C@@H](c3ccc(OCc4ccccc4)nc3)C2)nc(-c2ccc(C(F)(F)F)cc2F)c1C=O]

To a solution of ethyl 2-[(2S,6R)-2-(6-benzyloxy-3-pyridyl)-6-methyl-morpholin-4-yl]-5-(1,3-dioxolan-2-yl)-6-[2-fluoro-4-(trifluoromethyl)phenyl]pyrimidine-4-carboxylate (Int-D22, 1 equiv., 360 mg, 0.538 mmol) in acetone (6 mL) was added TsOH (0.2 equiv., 19 mg, 0.108 mmol), and the mixture was stirred at 60° C. for 1 h. The reaction solution was concentrated in vacuo to give ethyl 2-[(2S,6R)-2-(6-benzyloxy-3-pyridyl)-6-methyl-morpholin-4-yl]-6-[2-fluoro-4-(trifluoromethyl)phenyl]-5-formyl-pyrimidine-4-carboxylate (Int-D23, 330 mg, 0.528 mmol, 98% yield) as a solid. LCMS: [M+H]⁺=625.2. ¹H NMR (400 MHz, CDCl₃) δ 1.35 (br dd, J=11.9, 6.2 Hz, 3H) 1.40-1.48 (m, 3H) 2.81-3.04 (m, 2H) 3.83 (br dd, J=6.6, 3.5 Hz, 1H) 4.46-4.65 (m, 3H) 4.80-5.08 (m, 2H) 5.45 (br d, J=7.9 Hz, 2H) 6.91-6.98 (m, 1H) 7.34-7.52 (m, 6H) 7.57-7.71 (m, 2H) 7.85 (d, J=8.3 Hz, 1H) 8.38 (br d, J=4.9 Hz, 1H) 9.69 (d, J=3.8 Hz, 1H).

### Method Int 107. Intermediate Int-D24:2-[(2S,6R)-2-(6-benzyloxy-3-pyridyl)-6-methyl-morpholin-4-yl]-4-[2-fluoro-4-(trifluoromethyl)phenyl]-7H-pyrimido[4,5-d]pyridazin-8-one

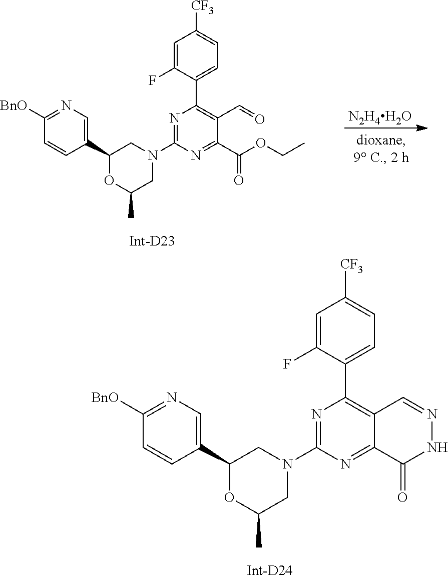 [SMILES: CCOC(=O)c1nc(N2C[C@@H](C)O[C@@H](c3ccc(OCc4ccccc4)nc3)C2)nc(-c2ccc(C(F)(F)F)cc2F)c1C=O ; "N2H4·H2O" ; C[C@@H]1CN(c2nc(-c3ccc(C(F)(F)F)cc3F)c3cn[nH]c(=O)c3n2)C[C@H](c2ccc(OCc3ccccc3)nc2)O1]

To a mixture of ethyl 2-[(2S,6R)-2-(6-benzyloxy-3-pyridyl)-6-methyl-morpholin-4-yl]-6-[2-fluoro-4-(trifluoromethyl)phenyl]-5-formyl-pyrimidine-4-carboxylate (Int-D23, 1 equiv., 330 mg, 0.528 mmol) in 1,4-dioxane (6 mL) was added N₂H₄·H₂O (2 equiv., 56 mg, 1.06 mmol), and the mixture was stirred for 2 hours at 90° C. The reaction solution was added into water (20 mL), extracted with EtOAc (20 mL×3). The organics was washed with 20 mL saturated brine solution. The organics were then separated and dried (Na₂SO₄) before concentration to dryness. The crude was purified by silica gel column (PE/EtOAc=3/1, R_{f}=0.5) to give 2-[(2S,6R)-2-(6-benzyloxy-3-pyridyl)-6-methyl-morpholin-4-yl]-4-[2-fluoro-4-(trifluoromethyl)phenyl]-7H-pyrimido[4,5-d]pyridazin-8-one (Int-D24, 220 mg, 0.371 mmol, 70% yield) as a solid. LCMS: [M+H]⁺=593.4. ¹H NMR (400 MHz, CDCl₃) δ 1.32-1.42 (m, 3H) 2.79-3.19 (m, 2H) 3.77-3.94 (m, 1H) 4.53-4.65 (m, 1H) 4.81-4.98 (m, 1H) 5.06-5.21 (m, 1H) 5.29-5.52 (m, 2H) 6.84 (d, J=8.5 Hz, 1H) 7.31-7.50 (m, 5H) 7.53-7.75 (m, 4H) 7.78 (d, J=3.5 Hz, 1H) 8.16-8.37 (m, 1H) 9.97-10.21 (m, 1H).

### Method Int 108. Intermediate Int-D26:2-nitroacetamide

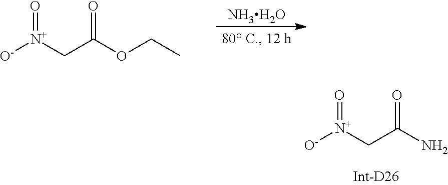

In a sealed tube, a mixture of ethyl 2-nitroacetate (1 equiv., 15.00 g, 113 mmol) in NH₃·H₂O (5.32 equiv., 50 mL, 600 mmol) was stirred at 80° C. for 12 h. The mixture was cooled to 20° C., poured into water (100 mL), and concentrated to half of the starting volume. The mixture was treated with HCl (1 N, 20 mL) to adjust pH=1~2 and extracted with EtOAc (300 mL×6). The organic layers were dried over Na₂SO₄, filtered, and concentrated under reduced pressure to give 2-nitroacetamide (10.00 g, 96.1 mmol, 85% yield) as a solid. ¹H NMR (400 MHz, DMSO-d₆) δ 7.85 (br s, 1H), 7.63 (br s, 1H), 5.28 (s, 2H).

### Method Int 109. Intermediate Int-D27: ethyl 6-methyl-3-nitro-2-oxo-1H-pyridine-4-carboxylate

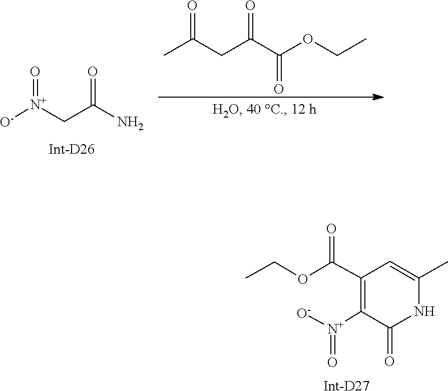

To a mixture of 2-nitroacetamide (1 equiv., 20.00 g, 192 mmol) and ethyl 2,4-dioxopentanoate (1.1 equiv., 33.44 g, 211 mmol) in water (300 mL) was added piperidinium acetate (1 equiv., 27.91 g, 192 mmol). The mixture was stirred at 40° C. for 12 h, extracted with EtOAc (500 mL×4), and concentrated under vacuum to give a residue. The residue was triturated in EtOAc (20 mL) and passed through a filter. The filtrate was concentrated under reduced pressure and purified by column chromatography (gradient elution of hexane/EtOAc=1:1~0:1) to obtain ethyl 6-methyl-3-nitro-2-oxo-1H-pyridine-4-carboxylate (Int-D27, 8.00 g, 35.4 mmol, 18% yield) as a solid. LCMS: [M+H]⁺=227.1. 1H NMR (400 MHz, DMSO-d₆) δ 13.45-12.81 (m, 1H), 6.41 (s, 1H), 4.29 (q, J=7.1 Hz, 2H), 2.32 (s, 3H), 1.24 (t, J=7.1 Hz, 3H).

### Method Int 110. Intermediate Int-D28:2,4-dihydroxy-6,7-dimethyl-pyrido[3,4-d]pyrimidin-8-one

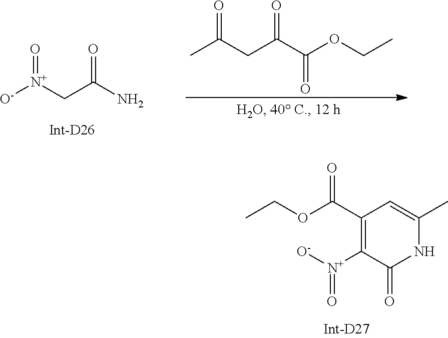 [SMILES: NC(=O)C[N+](=O)[O-] ; CCOC(=O)C(=O)CC(C)=O ; CCOC(=O)c1cc(C)[nH]c(=O)c1[N+](=O)[O-]]

To a suspension of 3-amino-1,6-dimethyl-2-oxo-pyridine-4-carboxylic acid (Int-D30, 1 equiv., 5000 mg, 27.4 mmol) in water (100 mL) was added AcOH (1.5 equiv., 2470 mg, 41.2 mmol), and the mixture was stirred at 40° C. for 15 min. Potassium cyanate (2.5 equiv., 5566 mg, 68.6 mmol) in water (30 mL) was added dropwise. The mixture was stirred at 40° C. for 1 h and then chilled to 0° C. NaOH (10 equiv., 10978 mg, 274 mmol) was added, and the stirring mixture was warmed up to 40° C. over 2 h. Water (50 mL) was added into the mixture, and pH was adjusted to 7 by adding HCl (aq.). The resulting slurry was filtered, and the filter cake was washed with water (10 mL×3). The combined filtrate was then concentrated to obtain 2,4-dihydroxy-6,7-dimethyl-pyrido[3,4-d]pyrimidin-8-one (5200 mg, 25.1 mmol, 91% yield) as a solid. LCMS: [M+H]⁺=208.0. ¹H NMR (400 MHz, DMSO-d₆) δ=11.92-10.20 (m, 2H), 6.47 (br s, 1H), 3.51 (br s, 3H), 2.37 (br s, 3H).

### Method Int 111. Intermediate Int-D32:2,4-dichloro-6,7-dimethyl-pyrido[3,4-d]pyrimidin-8-one

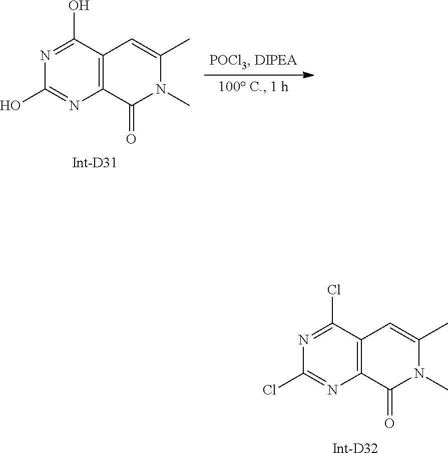

To a solution of 2,4-dihydroxy-6, 7-dimethyl-pyrido[3,4-d]pyrimidin-8-one (Int-D31, 1 equiv., 1000 mg, 4.83 mmol) in POCl₃ (24.7 equiv., 10 mL, 119 mmol) was added DIPEA (1 equiv., 624 mg, 4.83 mmol) at 0° C. under N₂. The mixture was stirred at 100° C. for 1 h under N₂ and then concentrated under reduced pressure to give a residue. EtOAc (20 mL) was added, and the mixture was added into NaHCO₃(aq., 150 mL) slowly below 40° C. and filtered to give crude starting material (0.7 g). The filtrate was extracted with EtOAc (100 mL×2). The organic layers were washed with brine (50 mL) and evaporated to give 2,4-dichloro-6,7-dimethyl-pyrido[3,4-d]pyrimidin-8-one (350 mg, 1.43 mmol, 29.71% yield) as a solid. LCMS: [M+H]⁺=244.0. ¹H NMR (400 MHz, CDCl₃) δ 6.59 (d, J=0.8 Hz, 1H), 3.68 (s, 3H), 2.53 (d, J=0.6 Hz, 3H).

### Method Int 112. Intermediate Int-D44:4-chloro-2-methyl-5-(3-(trifluoromethyl) bicyclo[1.1.1]pentane-1-carbonyl)pyridazin-3 (2H)-one

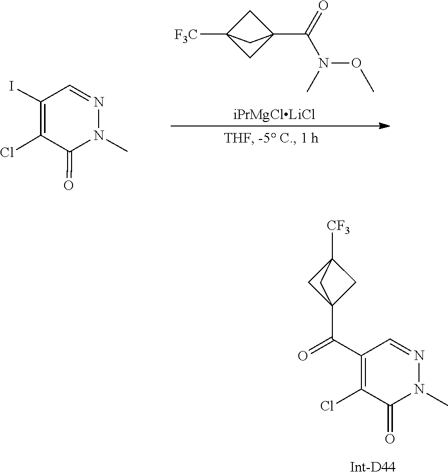

To a stirring solution of 4-chloro-5-iodo-2-methyl-pyridazin-3-one (1 equiv., 1.53 g, 5.65 mmol) and N-methoxy-N-methyl-3-(trifluoromethyl) bicyclo[1.1.1]pentane-1-carboxamide (Int-E4, 1 equiv., 2.67 g, 10.0 mmol) in anhydrous THF (15 mL), cooled to-5° C., was added isopropylmagnesium chloride lithium chloride complex (1.3 equiv., 13 mL, 13.0 mmol) dropwise. The reaction mixture was stirred at −5° C. for 1 h and then quenched with sat. NH₄Cl (aq)(2 mL) and warmed to r.t. The reaction mixture was diluted with EtOAc and washed with 5% citric acid (aq) and brine. The organic layer was dried over anhydrous Na₂SO₄, filtered and concentrated in vacuo. Purification by SiO₂ flash column chromatography using an elution gradient of 5-25% EtOAc in hexanes yielded 4-chloro-2-methyl-5-(3-(trifluoromethyl) bicyclo[1.1.1]pentane-1-carbonyl)pyridazin-3 (2H)-one (Int-D44, 915 mg, 2.98 mmol, 53% yield) as a solid. ¹H NMR (400 MHz, CDCl₃): δ 7.51 (s, 1H), 3.86 (s, 3H), 2.37 (s, 6H).

### Method Int 113. Intermediate Int-E1: ([4-amino-2-chloro-6-[3-(trifluoromethyl)-1-bicyclo[1.1.1]pentanyl]pyrimidin-5-yl]methanol

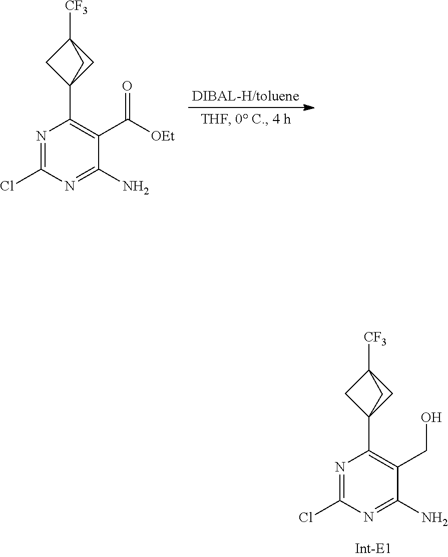 [SMILES: CCOC(=O)c1c(N)nc(Cl)nc1C12CC(C(F)(F)F)(C1)C2 ; Nc1nc(Cl)nc(C23CC(C(F)(F)F)(C2)C3)c1CO]

To a stirred solution of ethyl 4-amino-2-chloro-6-[3-(trifluoromethyl)-1-bicyclo[1.1.1]pentanyl]-pyrimidine-5-carboxylate (1 equiv., 1.49 g, 4.44 mmol) in THF (14.9 mL) at 0° C. was slowly added DIBAL-H (25% in toluene)(4 equiv., 10 mL, 17.8 mmol), and the reaction mixture was stirred in an ice-bath for 1 h. Sat. aqueous Rochelle salt (50 mL) was added, and the mixture was stirred at room temperature for 1 h. The resulting solution was extracted with EtOAc (3×50 mL), washed with brine, dried over MgSO₄, and filtered. The filtrate was concentrated under reduced pressure and purified by flash chromatography (Isco RediSep® column 40 g)(5-100% EtOAc in hexanes) to afford ([4-amino-2-chloro-6-[3-(trifluoromethyl)-1-bicyclo[1.1.1]pentanyl]pyrimidin-5-yl]methanol (Int-E1, 480 mg, 1.63 mmol, 55% yield) as a solid. ESI-MS: [M+H]⁺=292.0. ¹H NMR (CDCl₃, 400 MHz): δ 5.77 (2H, s), 4.79 (2H, d, =5.0 Hz), 2.41 (6H, s). ¹⁹F NMR (CDCl₃, 376 MHz): δ −73.2 (3F, s).

### Method Int 114. Intermediate Int-E2:4-amino-2-chloro-6-[3-(trifluoromethyl)-1-bicyclo[1.1.1]pentanyl]pyrimidine-5-carbaldehyde

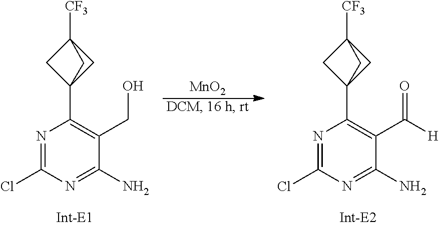

To a round-bottomed flask equipped with a Teflon-coated magnetic stirring bar was added [4-amino-2-chloro-6-[3-(trifluoromethyl)-1-bicyclo[1.1.1]pentanyl]pyrimidin-5-yl]methanol (Int-E1, 1 equiv., 806 mg, 2.74 mmol), and manganese (IV) oxide (20 equiv., 4.77 g, 54.9 mmol). The flask was sealed, purged under argon and dry DCM (21.6 mL) was added. The reaction mixture was stirred overnight at room temperature. The reaction mixture was filtered over a pad of Celite, rinsed with DCM (30 mL) and concentrated under reduced pressure to afford 4-amino-2-chloro-6-[3-(trifluoromethyl)-1-bicyclo[1.1.1]pentanyl]pyrimidine-5-carbaldehyde (Int-E2, 800 mg, 2.74 mmol, 99% yield) as a solid. ESI-MS: [M+H]⁺=292.1.

### Method Int 115. Intermediate Int-E3:2-chloro-6,7-dimethyl-4-[3-(trifluoromethyl)-1-bicyclo[1.1.1]pentanyl]pyrido[2,3-d]pyrimidine

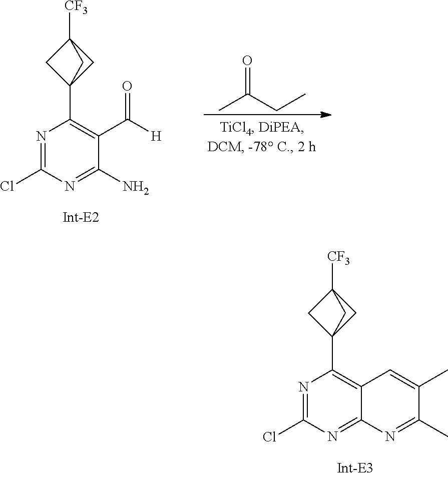

TiCl₄ (1.1 equiv., 0.10 mL, 0.90 mmol) was added to a stirring solution of butan-2-one (1 equiv., 77.0 μL, 0.86 mmol) in anhydrous DCM (3 mL) at −78° C. The reaction mixture was warmed up to 0° C. and stirred for 30 minutes. The solution was then cooled to-78° C., DIPEA (2.1 equiv., 0.31 mL, 1.77 mmol) was added dropwise, and the mixture was stirred at 0° C. for 30 minutes. The mixture was cooled to-30° C., a solution of 4-amino-2-chloro-6-[3-(trifluoromethyl)-1-bicyclo [1.1.1]pentanyl]pyrimidine-5-carbaldehyde (Int-E2, 1.2 equiv., 300 mg, 1.03 mmol) in anhydrous DCM (15 mL) was added dropwise and the mixture was stirred at 0° C. for 1 h. Sat. NH₄Cl (aq.)(10 mL) was added, and the resulting mixture was extracted with DCM (3× 10 mL). The combined organic layers were washed with water, brine, dried over anhydrous Na₂SO₄, filtered, and concentrated under reduced pressure. The residue was purified by flash chromatography (Isco RediSep® column 25 g)(0-30% EtOAc in hexanes) to afford 2-chloro-6,7-dimethyl-4-[3-(trifluoromethyl)-1-bicyclo[1.1.1]pentanyl]pyrido[2,3-d]pyrimidine (Int-E3, 54 mg, 0.16 mmol, 16% yield) as a solid. ESI-MS: [M+H]⁺=328.1. ¹H NMR (400 MHz, CDCl₃): δ 8.19 (s, 1H); 2.74 (s, 3H); 2.64 (s, 6H); 2.51 (s, 3H).

### Method Int 116. Intermediate Int-E5:2,2-dimethyl-N-[6-methyl-5-(trifluoromethyl)-2-pyridyl]propanamide

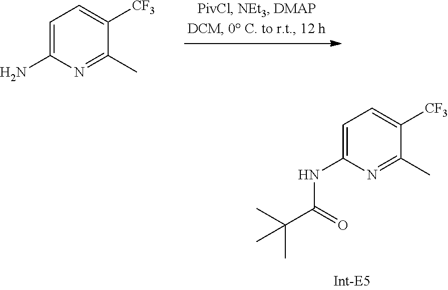 [SMILES: Cc1nc(N)ccc1C(F)(F)F ; Cc1nc(NC(=O)C(C)(C)C)ccc1C(F)(F)F]

To a flame-dried round-bottom flask equipped with a Teflon-coated magnetic stirring bar was added 6-methyl-5-(trifluoromethyl)pyridin-2-amine (1 equiv., 2.5 g, 14.2 mmol) and DMAP (0.1 equiv., 0.17 g, 1.42 mmol). The flask was capped, placed under an atmosphere of N₂, and anhydrous DCM (20 mL) and NEt₃ (1.5 equiv., 3.0 mL, 21.3 mmol) were added. The reaction mixture was cooled to 0° C. in an ice bath, and pivaloyl chloride (1.2 equiv., 2.1 mL, 17.0 mmol) was added, and the reaction mixture was allowed to warm to r.t. and stirred overnight. The reaction mixture was diluted with DCM (50 mL) washed with sat. NH₄Cl (aq.), and the aqueous layer was extracted with DCM (2×50 mL). The combined organic layers were dried with anhydrous Na₂SO₄, filtered, and concentrated under reduced pressure. The residue was purified by normal phase flash column chromatography (80 g SiO₂ column)(1-30% EtOAc in hexanes) to yield 2,2-dimethyl-N-[6-methyl-5-(trifluoromethyl)-2-pyridyl]propanamide (Int-E5, 3.62 g, 13.9 mmol, 98% yield) as a solid. ¹H NMR (CDCl₃, 400 MHz): δ 8.13 (1H, d, J=8.7 Hz), 8.08 (1H, s), 7.85 (1H, d, J=8.7 Hz), 2.56 (3H, s), 1.32 (9H, s). ¹⁹F NMR (CDCl₃, 376 MHz): δ −61.3 (3F, s).

### Method Int 117. Intermediate Int-E6:2,2-dimethyl-N-[6-methyl-5-(trifluoromethyl)-3-[3-(trifluoromethyl)-bicyclo[1.1.1]pentane-1-carbonyl]-2-pyridyl]propenamide

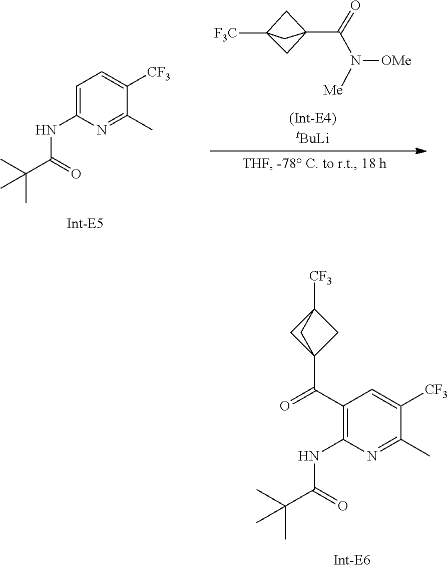 [SMILES: Cc1nc(NC(=O)C(C)(C)C)ccc1C(F)(F)F ; CON(C)C(=O)C12CC(C(F)(F)F)(C1)C2 ; "Int-E5" ; Cc1nc(NC(=O)C(C)(C)C)c(C(=O)C23CC(C(F)(F)F)(C2)C3)cc1C(F)(F)F ; "Int-E6"]

To a flame-dried round-bottom flask equipped with a magnetic stirring bar was added 2,2-dimethyl-N-[6-methyl-5-(trifluoromethyl)-2-pyridyl]propenamide (Int-E5, 1.1 equiv., 1.74 g, 6.7 mmol) and anhydrous THF (20 mL). The flask was sealed, purged under argon and cooled to-78° C. with stirring. ^{t}BuLi (1.7 M in pentane)(2.2 equiv., 7.9 mL, 13.4 mmol) was added dropwise. After 15 min, the flask was warmed to 0° C. and stirred for 1 h. The flask was cooled back to-78° C., and a solution of N-methoxy-N-methyl-3-(trifluoromethyl) bicyclo[1.1.1]pentane-1-carboxamide (Int-E4, 1 equiv., 1.36 g, 6.09 mmol) in anhydrous THF (10 mL) was added. The resulting mixture continued to stir at −78° C., gradually warming to r.t., and after 18 h was cooled to 0° C. and quenched with sat. NH₄Cl (aq.). The mixture was diluted with EtOAc and washed with sat. NH₄Cl (aq.) and brine. The organic layer was dried over anhydrous Na₂SO₄, filtered and concentrated in vacuo onto Celite (15 g). The crude was purified by normal phase flash column chromatography (200 g SiO₂ column)(5-20% EtOAc in hexanes) to yield 2,2-dimethyl-N-[6-methyl-5-(trifluoromethyl)-3-[3-(trifluoromethyl)-bicyclo[1.1.1]pentane-1-carbonyl]-2-pyridyl]propenamide (Int-E6, 757 mg, 1.79 mmol, 29% yield) as a solid.

An analogous procedure was followed to obtain the following intermediates.

| Structure | Starting Material | Characterization |
|---|---|---|
| 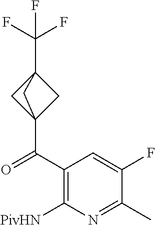 | 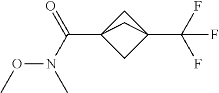 Int-E4 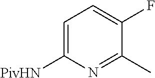 | The crude reaction mixture was purified by flash column chromatography (120 g SiO₂ column) using a 10-50% EtOAc in hexanes elution gradient to yield N-[5-fluoro- 6-methyl-3-[3-(trifluoromethyl)bicyclo[1.1.1]pentane-1- carbonyl]-2-pyridyl]-2,2-dimethyl-propanamide (Int- E20, 0.91 g, 2.44 mmol, 51% yield) as a solid. |
| Int-E20 | | |
| 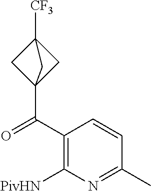 Int-E26 | 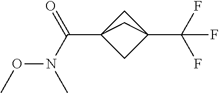 Int-E4 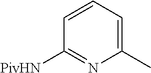 | The crude was purified by normal phase chromatography, 10-80% EtOAc in hexanes, to afford N-(6-methyl-3-(3- (trifluoromethyl)bicyclo[1.1.1]pentane-1- carbonyl)pyridin-2-yl)pivalamide (Int-E26, 400 mg, 56% yield) as a solid. ESI-MS: [M + H]⁺ = 355.2. ¹H NMR (CDCl₃, 400 MHz): δ 10.92 (1H, s), 8.08 (1H, d, J = 8.0 Hz), 6.93 (1H, d, J = 8.0 Hz), 2.60 (3H, s), 2.47 (6H, s), 1.34 (9H, s). |
| | Int-E25 | |
| 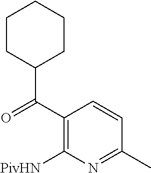 Int-E35 | 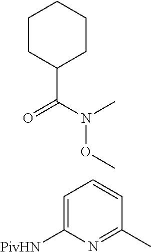 | The crude reaction mixture was purified by flash column chromatography (120 g SiO₂ column, 5-60% EtOAc in hexanes elution gradient) to afford N-[3- (cyclohexanecarbonyl)-6-methyl-2-pyridyl]-2,2- dimethyl-propanamide (2.3 g, 7.6 mmol, 38% yield) as a solid. |
| | Int-E25 | |

### Method Int 118. Intermediate Int-E7: [2-amino-6-methyl-5-(trifluoromethyl)-3-pyridyl]-[3-(trifluoromethyl)-1-bicyclo[1.1.1]pentanyl]methanone

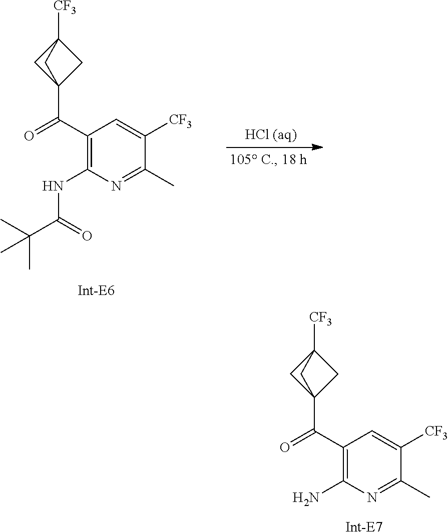

To a reaction vial equipped with a Teflon-coated magnetic stirring bar was added 2,2-dimethyl-N-[6-methyl-5-(trifluoromethyl)-3-[3-(trifluoromethyl) bicyclo[1.1.1]pentane-1-carbonyl]-2-pyridyl]-propanamide (Int-E6, 1 equiv., 123 mg, 0.29 mmol), water (3 mL) and 6N HCl (aq.)(21 equiv., 1.0 mL, 6.0 mmol), and the reaction mixture was stirred at 105° C. for 18 h. The resulting heterogeneous mixture was cooled in an ice bath, and IN NaOH solution (aq.) was added dropwise until pH=10 was obtained. The suspension was extracted with EtOAc (3×10 mL). The combined organic layers were dried over anhydrous Na₂SO₄, filtered, and concentrated under reduced pressure to afford [2-amino-6-methyl-5-(trifluoromethyl)-3-pyridyl]-[3-(trifluoromethyl)-1-bicyclo[1.1.1]pentanyl]methanone (Int-E7, 72 mg, 0.21 mmol, 73% yield) as a solid. ¹H NMR (CHCl₃, 400 MHz): δ 8.32 (s, 1H), 2.55 (s, 3H), 2.46 (s, 6H).

An analogous procedure was followed to obtain the following intermediates.

| Structure | Starting Material | Characterization |
|---|---|---|
| 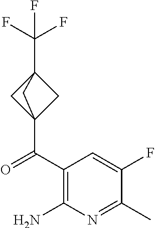 [SMILES: Cc1nc(N)c(C(=O)C23CC(C(F)(F)F)(C2)C3)cc1F] | 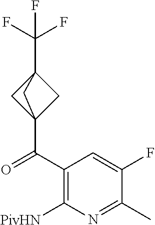 | The procedure afforded (2-amino-5-fluoro-6-methyl-3- pyridyl)-[3-(trifluoromethyl)-1- bicyclo[1.1.1]pentanyl]methanone (Int-E21, 704 mg, 2.44 mmol, 99% yield) as a solid. |
| Int-E21 | Int-E20 | |
| 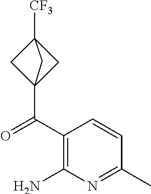 [SMILES: Cc1ccc(C(=O)C23CC(C(F)(F)F)(C2)C3)c(N)n1] | 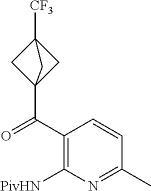 | The procedure afforded 2-amino-6-methylpyridin-3- yl)(3-(trifluoromethyl)bicyclo[1.1.1]pentan-1- yl)methanone (Int-E27, 142 mg, 90% yield) as a solid. ESI-MS: [M + H]⁺ = 272.1. ¹H NMR (CDCl₃, 400 MHz): δ 8.01 (1H, d, J = 8.0 Hz), 6.49 (1H, d, J = 8.0 Hz), 2.45 (6H, s), 2.41 (3H, s). |
| Int-E27 | Int-E26 | |
| 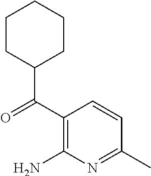 [SMILES: Cc1ccc(C(=O)C2CCCCC2)c(N)n1] | 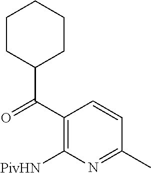 | The reaction crude was filtered through a silica plug to afford (2-amino-6-methyl-3-pyridyl)-cyclohexyl- methanone (Int-E36, 1.6 g, 7.3 mmol, 96% yield), which was used directly in the next step. |
| Int-E36 | Int-E35 | |

### Method Int 119. Intermediate Int-E8: (2R,4S)-2-(6-benzyloxy-3-pyridyl)-N-[6-methyl-5-(trifluoromethyl)-3-[3-(trifluoromethyl) bicyclo[1.1.1]-pentane-1-carbonyl]-2-pyridyl]tetrahydropyran-4-carboxamide

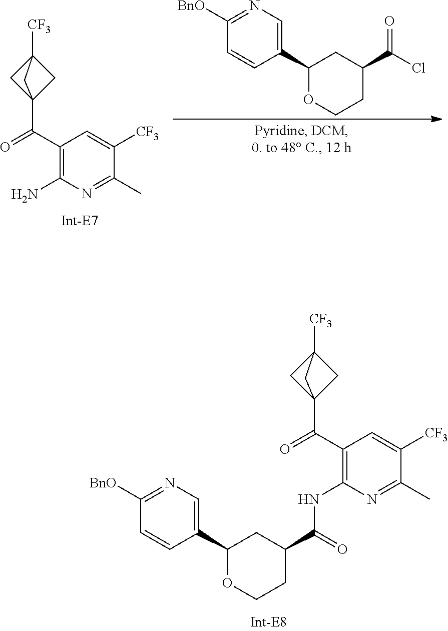

A flame-dried flask was charged with (2R,4S)-2-(6-benzyloxy-3-pyridyl)tetrahydropyran-4-carbonyl chloride (1.4 equiv., 100 mg, 0.3 mmol) and anhydrous DCM (1 mL) under an Ar atmosphere, and the resulting solution was stirred at 0° C. A solution of [2-amino-6-methyl-5-(trifluoromethyl)-3-pyridyl]-[3-(trifluoromethyl)-1-bicyclo[1.1.1]pentanyl]methanone (Int-E7, 1 equiv., 72 mg, 0.213 mmol) and pyridine (3.45 equiv., 0.06 mL, 0.74 mmol) in anhydrous DCM (1 mL) was then added dropwise, and the resulting mixture was stirred at 50° C. under argon overnight. The reaction mixture was concentrated under reduced pressure and dissolved in EtOAc (50 mL). The resulting solution was washed with 5% citric acid (aq.) and brine. The organic layer was collected, dried over anhydrous Na₂SO₄, filtered, and concentrated under reduced pressure. The residue was purified by normal phase flash column chromatography (12 g SiO₂ column)(10-60% EtOAc in hexanes) to yield (2R,4S)-2-(6-benzyloxy-3-pyridyl)-N-[6-methyl-5-(trifluoromethyl)-3-[3-(trifluoromethyl) bicyclo[1.1.1]-pentane-1-carbonyl]-2-pyridyl]tetrahydropyran-4-carboxamide (Int-E8, 30 mg, 0.05 mmol, 22% yield) as a solid. ¹H NMR (CDCl₃, 400 MHz): δ 10.98 (1H, s), 8.40 (1H, s), 8.14 (1H, s), 7.65 (1H, dd, J=8.5, 2.5 Hz), 7.29-7.44 (5H, m), 6.81 (1H, d, J=8.5 Hz), 5.38 (2H, s), 4.39 (1H, d, J=11.3 Hz), 4.27 (1H, d, J=11.6 Hz), 3.66-3.71 (1H, m), 3.12-3.14 (1H, m), 2.73 (3H, s), 2.49 (6H, s), 2.14 (1H, d, J=12.9 Hz), 1.82-1.97 (3H, m). ¹⁹F NMR (CDCl₃, 376 MHz): δ −61.8 (3F, s), −73.2 (3F, s).

An analogous procedure was followed to obtain the following intermediates.

| | Starting | |
|---|---|---|
| Structure | Material | Characterization |
| 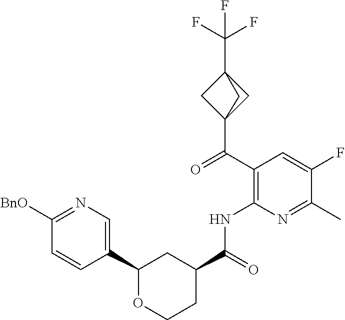 [SMILES: Cc1nc(NC(=O)[C@H]2CCO[C@@H](c3ccc(OCc4ccccc4)nc3)C2)c(C(=O)C23CC(C(F)(F)F)(C2)C3)cc1F] | 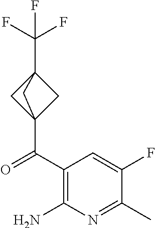 [SMILES: Cc1nc(N)c(C(=O)C23CC(C(F)(F)F)(C2)C3)cc1F] Int-E21 | The crude mixture was purified by flash column chromatography (40 g SiO₂ column) using an elution gradient of 50- 100% EtOAc in Hexanes to yield (2R,4S)-2-(6-benzyloxy-3-pyridyl)-N-[5- fluoro-6-methyl-3-[3- (trifluoromethyl)bicyclo[1.1.1]pentane-1- carbonyl]-2-pyridyl]tetrahydropyran-4- carboxamide (Int-E22, 213 mg, 0.365 mmol, 64% yield) as a solid. |
| Int-E22 | | |
| 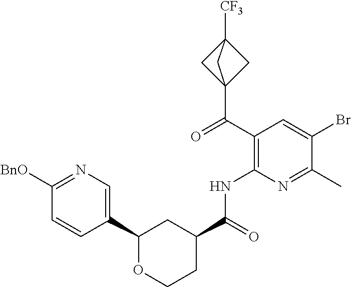 [SMILES: Cc1nc(NC(=O)[C@H]2CCO[C@@H](c3ccc(OCc4ccccc4)nc3)C2)c(C(=O)C23CC(C(F)(F)F)(C2)C3)cc1Br] | 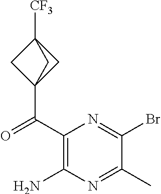 E28 | The crude was purified by normal phase chromatography (50-80% EtOAc in hexanes elution gradient) to afford (2R,4S)-2-(6-(benzyloxy)pyridin-3-yl)-N- (5-bromo-6-methyl-3-(3- (trifluoromethyl)bicyclo[1.1.1]pentane-1- carbonyl)pyridin-2-yl)tetrahydro-2H- pyran-4-carboxamide (Int-E29, 860 mg, 55% yield) as a solid. ESI-MS: [M + H]⁺ = 644.2. ¹H NMR (CDCl₃, 400 MHz): δ 10.59 (1H, s), 8.23 (1H, s), 8.13 (1H, d, J = 2.3 Hz), 7.64 (1H, dd, J = 8.6, 2.4 Hz), 7.45 (2H, d, J = 7.5 Hz), 7.37 (2H, t, J = 7.4 Hz), 7.29-7.33 (1H, m), 6.81 (1H, d, J = 8.6 Hz), 5.37 (2H, s), 4.38 (1H, d, J = 11.2 Hz), 4.24-4.28 (1H, m), 3.64-3.71 (1H, m), 2.96-3.04 (1H, m), 2.69 (3H, s), 2.48 (6H, s), 2.11-2.15 (1H, m), 1.93-2.02 |
| Int-E29 | | (2H, m). ¹⁹F NMR (CDCl₃, 376 MHz): δ |
| | | −73.2 (3F, s). |
| 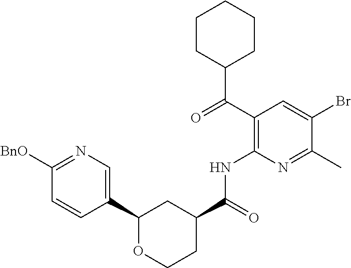 [SMILES: Cc1nc(NC(=O)[C@H]2CCO[C@@H](c3ccc(OCc4ccccc4)nc3)C2)c(C(=O)C2CCCCC2)cc1Br] | 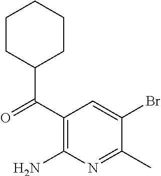 [SMILES: Cc1nc(N)c(C(=O)C2CCCCC2)cc1Br] Int-E37 | Modification: 4 equiv. of DIPEA was used instead of pyridine. The crude reaction mixture was purified by flash column chromatography (120 g SiO₂ column, 0-35% EtOAc in DCM elution gradient) to yield (2R,4S)-2-(6- benzyloxy-3-pyridyl)-N-[5-bromo-3- (cyclohexanecarbonyl)-6-methyl-2- pyridyl]tetrahydropyran-4-carboxamide (1.3 g, 2.2 mmol, 69% yield) as a solid. |
| Int-E38 | | |

### Method Int 120. Intermediate Int-E12:2-chloro-4-(3,3-difluorocyclobutoxy)-6,7-dimethylpyrido[2,3-d]pyrimidine

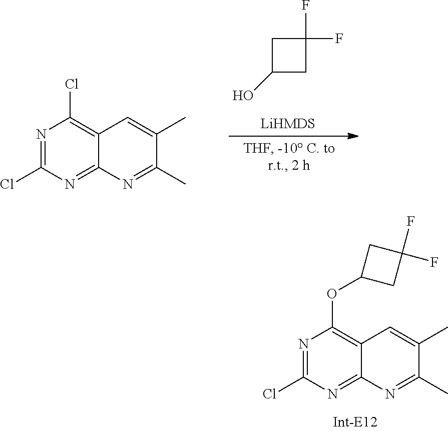 [SMILES: Cc1cc2c(Cl)nc(Cl)nc2nc1C ; OC1CC(F)(F)C1 ; Cc1cc2c(OC3CC(F)(F)C3)nc(Cl)nc2nc1C]

Lithium bis(trimethylsilyl)amide (1 equiv., 0.88 mL, 0.88 mmol) was added to a stirring solution of 3,3-difluorocyclobutanol (1 equiv., 0.1 mL, 0.88 mmol) in dry THF (17.5 mL) at −10° C. and stirred for 30 min at the same temperature. The resulting solution was then added dropwise to a solution of 2,4-dichloro-6,7-dimethylpyrido[2,3-d]pyrimidine (1 equiv., 200 mg, 0.88 mmol) in THF dry (1 mL). The mixture was stirred for 2 h at room temperature. The reaction was quenched with water (50 mL) and extracted with EtOAc (3×50 mL). The combined organic layers were washed with brine, dried over anh. Na₂SO₄, filtered, and concentrated under reduced pressure. The crude material was purified by flash chromatography (Isco RediSep® column 12 g, using a gradient from 0% to 60% EtOAc in hexanes). The selected fractions were evaporated to provide 2-chloro-4-(3,3-difluorocyclobutoxy)-6,7-dimethylpyrido[2,3-d]pyrimidine (Int-E12, 198 mg, 0.66 mmol, 75%) as a solid. ¹H NMR (CDCl₃, 400 MHz): δ 8.12 (s, 1H), 5.49-5.38 (m, 1H), 3.22-3.31 (m, 1H), 2.83-2.95 (m, 1H), 2.74 (s, 3H), 2.48 (s, 3H). ESI-MS: [M+H]⁺=366.0.

### Method Int 121. Intermediate Int-E17:4-amino-6-(2,4-difluorophenyl)-2-((2R,4S)-2-(6-oxo-1,6-dihydropyridin-3-yl)tetrahydro-2H-pyran-4-yl)pyrimidine-5-carbaldehyde

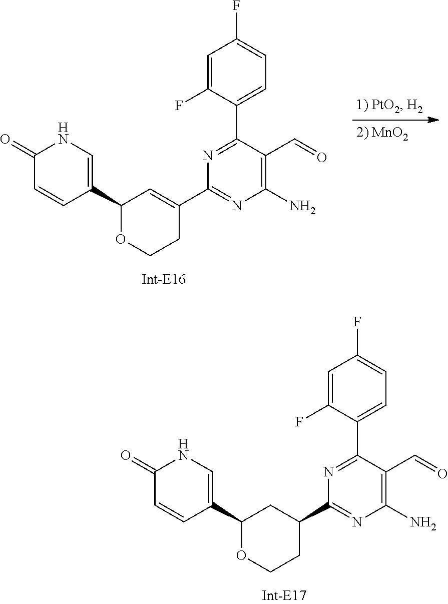 [SMILES: Nc1nc(C2=C[C@H](c3ccc(=O)[nH]c3)OCC2)nc(-c2ccc(F)cc2F)c1C=O ; Nc1nc([C@H]2CCO[C@@H](c3ccc(=O)[nH]c3)C2)nc(-c2ccc(F)cc2F)c1C=O]

To a solution of (R)-4-amino-6-(2,4-difluorophenyl)-2-(6-(6-oxo-1,6-dihydropyridin-3-yl)-3,6-dihydro-2H-pyran-4-yl)pyrimidine-5-carbaldehyde (Int-E16, 1 equiv., 480 mg, 1.17 mmol) in ethanol (10 mL) was added platinum dioxide (0.6 eq, 159 mg, 0.70 mmol). The system was purged with hydrogen, and the mixture was stirred at 25° C. for 4 h under a balloon of hydrogen. The reaction was then purged with nitrogen, diluted with DCM, filtered thought celite and evaporated under vacuum. The residue was diluted in DCM (10 mL) and manganese dioxide (10 eq, 2.7 g, 11.7 mmol) was added. The resulting mixture was stirred at room temperature overnight then filtered through celite and evaporated. The residue was purified by column chromatography (80 g, SiO₂) using a 0-20% MeOH in DCM gradient to yield 4-amino-6-(2,4-difluorophenyl)-2-((2R,4S)-2-(6-oxo-1,6-dihydropyridin-3-yl)tetrahydro-2H-pyran-4-yl)pyrimidine-5-carbaldehyde (Int-E17, 240 mg, 0.58 mmol, 50%) as a foam.

### Method Int 122. Intermediate Int-E18: methyl 4-(2,4-difluorophenyl)-7-methyl-2-((2R,4S)-2-(6-oxo-1,6-dihydropyridin-3-yl)tetrahydro-2H-pyran-4-yl) pyrido[2,3-d]pyrimidine-6-carboxylate

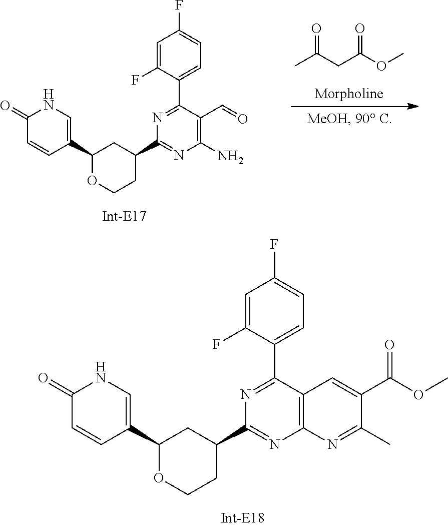

To a solution of methyl 3-oxobutanoate 7 (2 equiv., 59 mg, 0.51 mmol) in methanol (1 mL) was added morpholine (1 equiv., 22 μL, 0.26 mmol). The mixture was stirred for 30 min and then added to a solution of 4-amino-6-(2,4-difluorophenyl)-2-[(2R,4S)-2-(6-oxo-1H-pyridin-3-yl)tetrahydropyran-4-yl]pyrimidine-5-carbaldehyde (Int-E17, 1 eq, 105 mg, 0.26 mmol) in methanol (1 mL). The solution was stirred overnight at 90° C. in a sealed vessel. The mixture was cooled down, diluted with AcOEt, washed with aqueous (10%) AcOH, brine, dried over Na₂SO₄, filtered, and evaporated. The residue was purified on a 40 g silica column using a 0-20% MeOH in DCM elution gradient to yield methyl 4-(2,4-difluorophenyl)-7-methyl-2-((2R,4S)-2-(6-oxo-1,6-dihydropyridin-3-yl)tetrahydro-2H-pyran-4-yl) pyrido[2,3-d]pyrimidine-6-carboxylate (Int-E18, 68 mg, 0.14 mmol, 54%) as a foam.

### Method Int 123. Intermediate Int-E25: N-(6-methylpyridin-2-yl) pivalamide

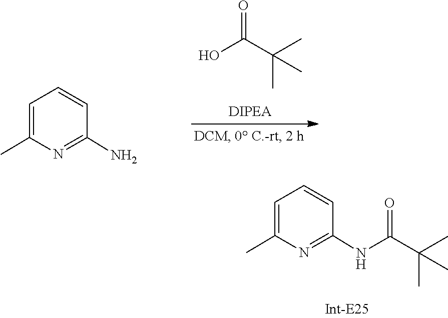

To a flame-dried round-bottomed flask equipped with a Teflon-coated magnetic stirring bar under nitrogen atmosphere was added 6-methylpyridin-2-amine (1 equiv., 30.0 g, 277 mmol), DCM (60 mL) and triethylamine (1.5 equiv., 58 mL, 416 mmol). The flask was cooled and pivaloyl chloride (1.2 equiv., 41 mL, 333 mmol) was added dropwise over 4 minutes. The mixture was stirred for 3 h. The reaction mixture was washed with water (300 mL) and extracted with EtOAc (3×500 mL). The organic layers were combined, washed with brine, filtered, and concentrated under reduced pressure. The crude material was purified by flash chromatography, 0-50% EtOAc in hexanes elution gradient to afford N-(6-methylpyridin-2-yl) pivalamide as a solid (Int-E25, 50.0 g, 94% yield). ESI-MS: [M+H]⁺=193.2. ¹H NMR (CDCl₃, 400 MHz): δ 8.03 (1H, d, J=8.3 Hz), 7.89 (1H, s), 7.56 (1H, t, J=7.9 Hz), 6.86 (1H, d, J=7.5 Hz), 2.43 (3H, s), 1.31 (9H, s).

### Method Int 124. Intermediate Int-E28: (2-amino-5-bromo-6-methylpyridin-3-yl)(3-(trifluoromethyl) bicyclo[1.1.1]pentan-1-yl) methanone

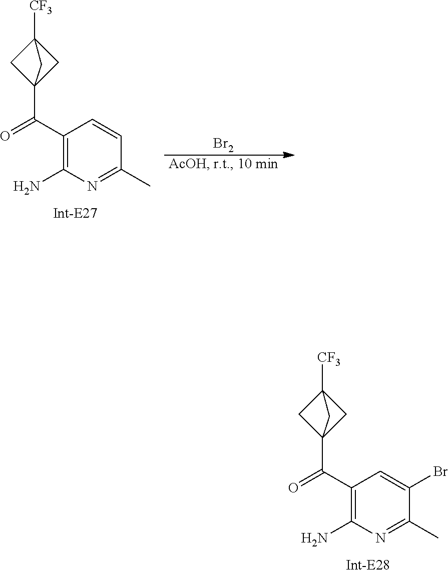

To a glass vial equipped with a Teflon-coated magnetic stirring bar was added (2-amino-6-methyl-3-pyridyl)-[3-(trifluoromethyl)-1-bicyclo[1.1.1]pentanyl]methanone (Int-E27, 1 equiv., 203 mg, 0.75 mmol) and acetic acid (2 mL). Bromine (1.1 equiv., 43 μL, 0.83 mmol) was added while stirring at room temperature. After 10 min., the reaction mixture was diluted with H₂O (2 mL) and basified to pH 10 with 1 M NaOH (aq). The resulting precipitate was collected via vacuum filtration, rinsed with H₂O and dried under reduced pressure. The crude was purified by normal phase chromatography, 5% to 20% EtOAc in hexanes, to afford (2-amino-5-bromo-6-methylpyridin-3-yl)(3-(trifluoromethyl) bicyclo[1.1.1]pentan-1-yl) methanone (Int-E28, 217 mg, 83% yield) as a solid. ESI-MS: [M+H]⁺=351.3. ¹H NMR (CDCl₃, 400 MHz): δ H 8.15 (1H, s), 2.52 (3H, s), 2.46 (6H, s).

An analogous procedure was followed to obtain the following intermediates.

| Structure | Starting Material | Characterization |
|---|---|---|
| 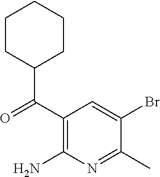 [SMILES: Cc1nc(N)c(C(=O)C2CCCCC2)cc1Br] | 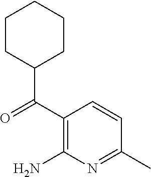 [SMILES: Cc1ccc(C(=O)C2CCCCC2)c(N)n1] | The product was purified by flash column chromatography (120 g SiO₂ column, 5-20% EtOAc in hexanes elution gradient to afford (2-amino-5-bromo-6- methyl-3-pyridyl)-cyclohexyl-methanone (Int-E37, 1.9 g, 6.4 mmol, 87% yield). |
| Int-E37 | Int-E36 | |

### Method Int 125. Intermediate Int-E33:2-((2R,4S)-2-(6-(benzyloxy)pyridin-3-yl)tetrahydro-2H-pyran-4-yl)-7-methyl-4-(3-(trifluoromethyl) bicyclo[1.1.1]pentan-1-yl) pyrido[2,3-d]pyrimidine-6-carbonitrile

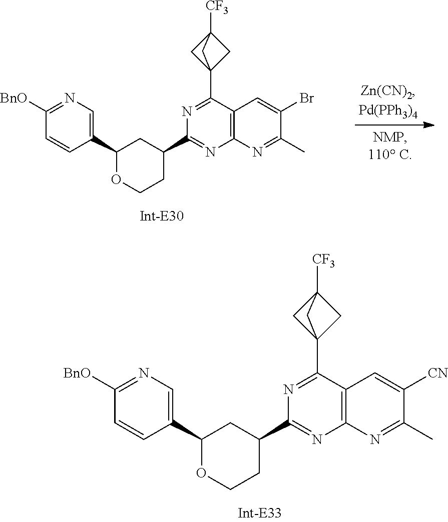 [SMILES: Cc1nc2nc([C@H]3CCO[C@@H](c4ccc(OCc5ccccc5)nc4)C3)nc(C34CC(C(F)(F)F)(C3)C4)c2cc1Br ; Cc1nc2nc([C@H]3CCO[C@@H](c4ccc(OCc5ccccc5)nc4)C3)nc(C34CC(C(F)(F)F)(C3)C4)c2cc1C#N]

2-[(2R,4S)-2-(6-benzyloxy-3-pyridyl)tetrahydropyran-4-yl]-6-bromo-7-methyl-4-[3-(trifluoromethyl)-1-bicyclo[1.1.1]pentanyl]pyrido[2,3-d]pyrimidine (Int-E30, 1 equiv., 100 mg, 0.16 mmol), zinc cyanide (2 equiv., 38 mg, 0.32 mmol) and Pd(PPh₃)₄ (0.10 equiv., 18 mg, 0.02 mmol) were stirred at 110° C. in NMP (1.5 mL) for 3 h. The reaction diluted with water and extracted with EtOAc (2× 20 mL). The combined organic layers were washed with brine, dried over Na₂SO₄, filtered, and concentrated under reduced pressure. The crude was purified by normal phase chromatography (50-100% EtOAc in hexane elution gradient) to obtain 2-((2R,4S)-2-(6-(benzyloxy)pyridin-3-yl)tetrahydro-2H-pyran-4-yl)-7-methyl-4-(3-(trifluoromethyl) bicyclo[1.1.1]pentan-1-yl) pyrido[2,3-d]pyrimidine-6-carbonitrile (Int-E33, 80 mg, 87% yield) as a solid. ESI-MS: [M+H]⁺=572.3. ¹H NMR (CDCl₃, 400 MHz): δ 8.80 (1H, s), 8.15 (1H, s), 7.66 (1H, dd, J=8.5, 2.4 Hz), 7.42 (2H, d, J=7.5 Hz), 7.33 (2H, t, J=7.4 Hz), 6.78 (1H, d, J=8.5 Hz), 5.35 (2H, s), 4.51 (1H, d, J=11.2 Hz), 4.30 (1H, d, J=11.4 Hz), 3.75-3.85 (1H, m), 3.46-3.53 (1H, m), 2.99 (3H, s), 2.64 (6H, s), 2.29 (1H, d, J=13.2 Hz), 2.12-2.17 (2H, m), 2.07 (1H, t, J=12.2 Hz). ¹⁹F NMR (CDCl₃, 376 MHz): δ −73.1 (3F, s).

An analogous procedure was followed to obtain the following intermediates.

| Structure | Starting Material | Characterization |
|---|---|---|
| 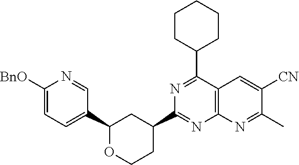 [SMILES: Cc1nc2nc([C@H]3CCO[C@@H](c4ccc(OCc5ccccc5)nc4)C3)nc(C3CCCCC3)c2cc1C#N] | 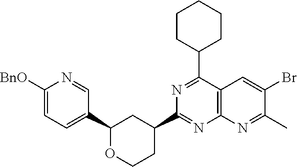 [SMILES: Cc1nc2nc([C@H]3CCO[C@@H](c4ccc(OCc5ccccc5)nc4)C3)nc(C3CCCCC3)c2cc1Br] | The crude mixture was purified by flash column chromatography using 50-100% EtOAc in hexanes elution gradient to afford 2-[(2R,4S)-2-(6- benzyloxy-3-pyridyl)tetrahydropyran- 4-yl]-4-cyclohexyl-7-methyl- pyrido[2,3-d]pyrimidine-6-carbonitrile (Int-E40, 390 mg, 0.75 mmol, 86% yield) as a solid. |
| Int-E40 | Int-E39 | |

### Method Int 126. Intermediate Int-E42: [6-(1-methyl-6-oxo-3-pyridyl)-3,6-dihydro-2H-pyran-4-yl]trifluoromethanesulfonate

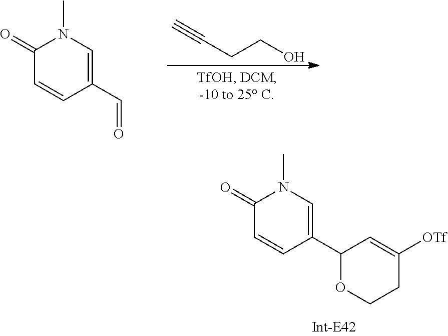

To 1-methyl-6-oxo-pyridine-3-carbaldehyde (1 equiv., 3500 mg, 25.5 mmol) in DCM (40 mL) was added 3-butyn-1-ol (1 equiv., 1.9 mL, 25.5 mmol), and the mixture was stirred at −15° C. for 30 min under nitrogen atmosphere. TfOH (3 equiv., 6.8 mL, 76.6 mmol) was added dropwise at −15 to-5° C. under nitrogen atmosphere, and the mixture was stirred the mixture at −10 to 0° C. for 1 h, then stirred at 10 to 20° C. for 16 h. The reaction mixture was poured into water (100 mL), extracted with EtOAc (100 mL×3). The combined organic phase was washed by brine (50 mL), dried by Na₂SO₄, purified by reverse flash chromatography, and lyophilized to give [6-(1-methyl-6-oxo-3-pyridyl)-3,6-dihydro-2H-pyran-4-yl]trifluoromethanesulfonate (Int-E42, 2.40 g, 7.07 mmol, 28% yield) as an oil. ESI-MS: [M+H]⁺=340.0. ¹H NMR (400 MHz, CDCl₃) δ 7.35 (dd, J=2.6, 9.3 Hz, 1H), 7.30 (d, J=2.4 Hz, 1H), 6.64 (d, J=9.4 Hz, 1H), 5.86-5.81 (m, 1H), 5.07 (q, J=2.6 Hz, 1H), 4.06 (ddd, J=3.9, 5.4, 11.6 Hz, 1H), 3.84-3.80 (m, 1H), 3.56 (s, 3H), 2.70-2.63 (m, 1H), 2.47-2.36 (m, 1H).

### Method Int 127. Intermediate Int-E43:1-methyl-5-[4-(4,4,5,5-tetramethyl-1,3,2-dioxaborolan-2-yl)-3,6-dihydro-2H-pyran-6-yl]pyridin-2-one

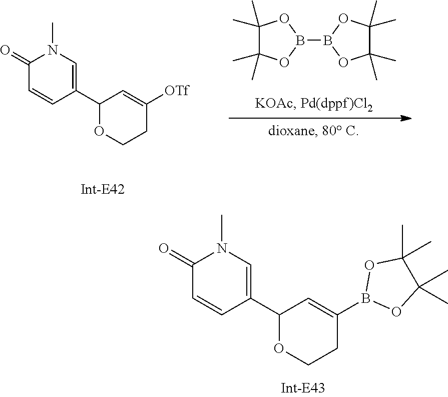

To a solution of [6-(1-methyl-6-oxo-3-pyridyl)-3,6-dihydro-2H-pyran-4-yl]trifluoromethanesulfonate (Int-E42, 1 equiv., 1.40 g, 4.13 mmol) in dioxane (14 mL) was added bis(pinacolato)diboron (1.5 equiv., 1572 mg, 6.19 mmol) and potassium acetate (4 equiv., 1620 mg, 16.5 mmol), and then the mixture was stirred at 25° C. under N₂ atmosphere for 30 min. Pd(dppf)Cl₂ (0.0300 eq, 91 mg, 0.124 mmol) was added, and the mixture was heated at 80° C. for 4 h under N₂ atmosphere. The reaction mixture was poured into water (60 mL), extracted with EtOAc (50 mL×3). The combined organic phase was washed by brine (20 mL), dried by Na₂SO₄, purified by reverse flash chromatography, and lyophilized to give 1-methyl-5-[4-(4,4,5,5-tetramethyl-1,3,2-dioxaborolan-2-yl)-3,6-dihydro-2H-pyran-6-yl]pyridin-2-one (Int-E43, 240 mg, 0.757 mmol, 18% yield) as an oil. ESI-MS: [M+H]⁺=318.1. 1H NMR (400 MHz, CDCl₃) δ 8.07 (d, J=1.0 Hz, 1H), 7.38 (br d, J=9.3 Hz, 1H), 7.30 (d, J=1.4 Hz, 1H), 6.62 (br d, J=9.4 Hz, 1H), 6.45 (s, 1H), 4.94 (br d, J=1.8 Hz, 1H), 4.00-3.92 (m, 1H), 3.77-3.69 (m, 1H), 3.56 (s, 3H), 2.41-2.33 (m, 1H), 1.29 (s, 12H).

### Method Int 128. Intermediate Int-F5: (R)-2-(6-(benzyloxy)pyridin-3-yl)tetrahydro-4H-pyran-4-one

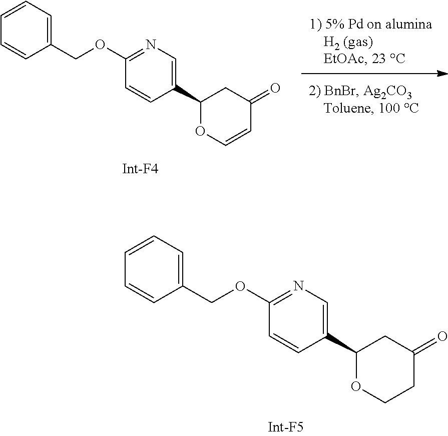

2-(6-benzyloxy-3-pyridyl)-2,3-dihydropyran-4-one (Int-F4, 1 equiv., 0.90 g, 3.20 mmol) was charged in a vial with EtOAc (20 mL, 0.16 M), and the solution was purged with argon before palladium on alumina (0.03 equiv., 0.20 g, 0.096 mmol) was added. The reaction mixture was purged with hydrogen three times and the mixture was stirred overnight at r.t. under 1 atm of H₂ (g). The mixture was filtered through a celite pad and concentrated in vacuo. Re-benzylation procedure: The reaction residue was diluted in toluene (20 mL, 0.16 M) and, benzyl bromide (1.2 equiv., 0.66 g, 3.84 mmol) and silver carbonate (1.5 equiv., 1.32 g, 4.80 mmol) were added. The resulting mixture was stirred at 100° C. for 1 h, filtered through a celite pad, and concentrated in vacuo. The crude was purified by normal phase chromatography (0-20% EtOAc in DCM) to provide (R)-2-(6-(benzyloxy)pyridin-3-yl)tetrahydro-4H-pyran-4-one as a solid (Int-F5, 460 mg, (51% yield over two steps). ¹H NMR (CDCl₃, 400 MHz): δ 8.15 (1H, d, J=2.4 Hz), 7.64 (1H, dd, J=8.6, 2.4 Hz), 7.45 (2H, d, J=7.5 Hz), 7.37 (2H, t, J=7.3 Hz), 7.32 (1H, d, J=7.0 Hz), 6.84 (1H, d, J=8.6 Hz), 5.39 (2H, s), 4.63 (1H, dd, J=9.0, 5.2 Hz), 4.42 (1H, dd, J=11.5, 7.4 Hz), 3.84 (1H, td, J=11.9, 2.8 Hz), 2.67-2.76 (1H, m), 2.64 (1H, d, J=4.3 Hz), 2.44 (1H, d, J=14.7 Hz). ESI-MS: [M+H]⁺=284.3.

### Method Int 129. Intermediate Int-F10: methyl 4-acetylbicyclo[2.2.1]heptane-1-carboxylate

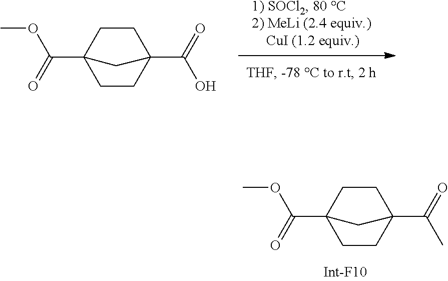

Excess thionyl chloride was reacted with 4-(methoxycarbonyl) bicyclo[2.2.1]heptane-1-carboxylic acid at 80° C. for 5 h, and then separated to obtain an intermediate acid chloride. The acid chloride was dissolved in THF, chilled to-78° C., and treated with CuI (1.2 equiv.) and MeLi (2.4 equiv.). The reaction was allowed to warm to r.t. over two hours affording methyl 4-acetylbicyclo[2.2.1]heptane-1-carboxylate (Int-F10).

### Method Int 130. Intermediate Int-F12:4-(1,1-difluoroethyl)-N-methoxy-N-methylbicyclo[2.2.1]heptane-1-carboxamide

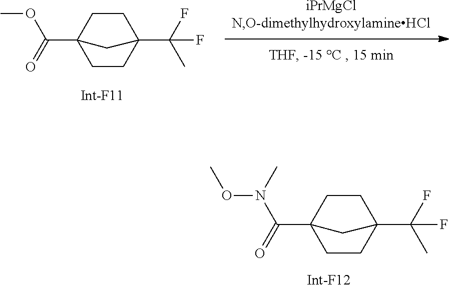 [SMILES: COC(=O)C12CCC(C(C)(F)F)(CC1)C2 ; "iPrMgClN,O-dimethylhydroxylamine·HCl" ; CON(C)C(=O)C12CCC(C(C)(F)F)(CC1)C2]

Methyl 4-(1,1-difluoroethyl) bicyclo[2.2.1]heptane-1-carboxylate (Int-F11, 1 equiv.), iPrMgCl (2M solution in THF, 2.4 equiv.), and N,O-dimethylhydroxylamine hydrochloride (1.2 equiv.) were reacted in THF at −15° C. for 15 min to afford 4-(1,1-difluoroethyl)-N-methoxy-N-methylbicyclo[2.2.1]heptane-1-carboxamide (Int-F12).

### Example A2: Synthesis of Exemplary Compounds

### Method 1: Synthesis of Example 7, 16, and 62

### Example 7:5-[(2S,6R)-4-[4-[2-fluoro-4-(trifluoromethyl)phenyl]-6,7-dimethyl-pteridin-2-yl]-6-methyl-morpholin-2-yl]-1-methyl-pyridin-2-one

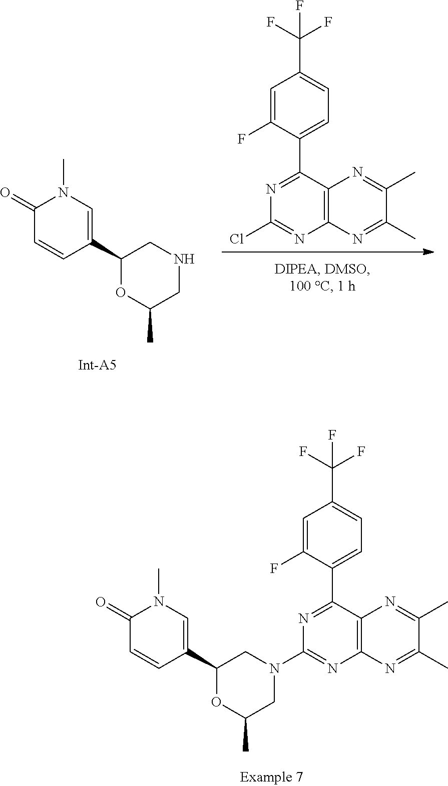 [SMILES: C[C@@H]1CNC[C@H](c2ccc(=O)n(C)c2)O1 ; Cc1nc2nc(Cl)nc(-c3ccc(C(F)(F)F)cc3F)c2nc1C ; Cc1nc2nc(N3C[C@@H](C)O[C@@H](c4ccc(=O)n(C)c4)C3)nc(-c3ccc(C(F)(F)F)cc3F)c2nc1C]

To a solution of 2-chloro-4-[2-fluoro-4-(trifluoromethyl)phenyl]-6,7-dimethyl-pteridine (1 equiv., 50 mg, 0.14 mmol) and 1-methyl-5-[(2S,6R)-6-methylmorpholin-2-yl]pyridin-2-one (Int-A5, 2 equiv., 58 mg, 0.28 mmol) in DMSO (0.5 mL), was added DIPEA (5 equiv., 0.12 mL, 0.7 mmol), and the reaction was stirred at 100° C. for 1 h. LCMS showed 87% desired product. The reaction mixture was poured into water (10 mL) and then extracted with EtOAc (3×20 mL). The combined organic layers were dried over Na₂SO₄, filtered, and concentrated under reduced pressure to give a residue. The residue was purified by prep-HPLC (Condition 2, Gradient a) to afford 5-[(2S,6R)-4-[4-[2-fluoro-4-(trifluoromethyl)phenyl]-6,7-dimethyl-pteridin-2-yl]-6-methyl-morpholin-2-yl]-1-methyl-pyridin-2-one (Example 7, 40 mg, 0.075 mmol, 53% yield) as a solid. LCMS: [M+H]⁺=529.2; purity=99% (220 nm); Ret. time=0.953 min. ¹H NMR (400 MHz, CDCl₃) δ 7.81 (brt, J=7.2 Hz, 1H), 7.60 (br d, J=7.9 Hz, 1H), 7.54-7.48 (m, 1H), 7.43 (s, 2H), 6.61 (d, J=9.4 Hz, 1H), 5.24-4.88 (m, 2H), 4.47-4.36 (m, 1H), 3.93-3.76 (m, 1H), 3.58 (s, 3H), 2.97-2.81 (m, 2H), 2.73 (s, 3H), 2.60 (s, 3H), 1.36 (s, 1H), 1.39-1.31 (m, 1H).

An analogous procedure was followed to obtain the following compounds.

| Structure | Starting Material | Characterization |
|---|---|---|
| 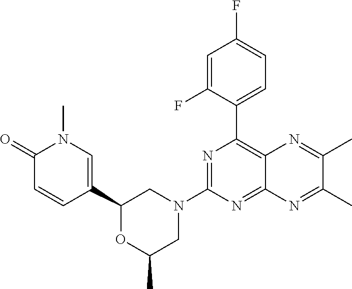 [SMILES: Cc1nc2nc(N3C[C@@H](C)O[C@@H](c4ccc(=O)n(C)c4)C3)nc(-c3ccc(F)cc3F)c2nc1C] | 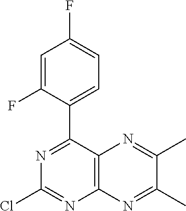 [SMILES: Cc1nc2nc(Cl)nc(-c3ccc(F)cc3F)c2nc1C] | The residue was purified by Prep-TLC (50% EtOAc in petroleum ether (PE), R_{f} = 0.1) to give the crude product. The crude product was purified again by Prep-HPLC (0.5% FA) and extracted with EtOAc (3 × 20 mL). The combined organic layer was dried over anhydrous Na₂SO₄, filtered, and concentrated under reduced pressure to give the residue. The residue was purified again by Prep- HPLC (Condition 3) and lyophilized to give the residue. The residue was purified again by Prep-TLC (100% EtOAc) to give the 5-[(2S,6R)-4-[4- (2,4-difluorophenyl)-6,7-dimethyl- pteridin-2-yl]-6-methyl-morpholin-2- yl]-1-methyl-pyridin-2-one (Example 16, 5.5 mg, 0.011 mmol, 7% yield) as a |
| Example 16 | | solid. LCMS: [M + H]⁺ = 479.2; purity = |
| | | 98% (220 nm); Ret. time = 0.531 min. |
| | | ¹H NMR (400 MHz, CDCl₃) δ 7.72 (q, J = |
| | | 7.5 Hz, 1H), 7.50-7.38 (m, 2H), 7.09- |
| | | 7.02 (m, 1H), 6.99 (dt, J = 2.4, 9.4 Hz, |
| | | 1H), 6.61 (d, J = 9.3 Hz, 1H), 5.15-4.91 |
| | | (m, 2H), 4.45-4.33 (m, 1H), 3.90-3.79 |
| | | (m, 1H), 3.58 (s, 3H), 2.93 (dd, J = 10.9, |
| | | 13.3 Hz, 1H), 2.83 (dd, J = 10.8, 13.3 |
| | | Hz, 1H), 2.73 (s, 3H), 2.60 (s, 3H), 1.38- |
| | | 1.32 (m, 3H). |
| 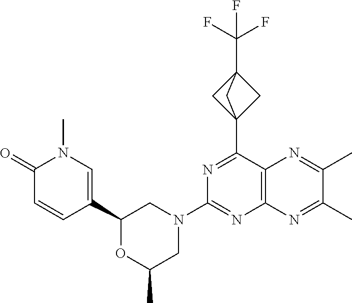 [SMILES: Cc1nc2nc(N3C[C@@H](C)O[C@@H](c4ccc(=O)n(C)c4)C3)nc(C34CC(C(F)(F)F)(C3)C4)c2nc1C] | 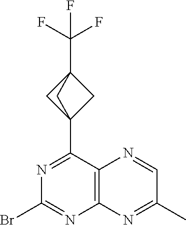 | Modification: The reaction was ran with DIPEA at 110 °C for 2 hours. The reaction mixture was filtered and the filtrate was purified by prep-HPLC (Condition 4) and lyophilized to give 5- [(2S,6R)-4-[6,7-dimethyl-4-[3- (trifluoro-methyl)-1- bicyclo[1.1.1]pentanyl]pteridin-2-yl]- 6-methyl-morpholin-2-yl]-1-methyl- pyridin-2-one (Example 62, 3.7 mg, 0.007 mmol, 14% yield) as a solid. LCMS: [M + H]⁺ = 501.4, purity = 100% (220 nm); Ret. time = 0.608 min. ¹H NMR (400 MHZ, CDCl₃) δ 7.49-7.40 (m, 2H), 6.62 (br d, J = 9.5 Hz, 1H), 5.10- 4.89 (m, 2H), 4.37 (dd, J = 2.4, 10.7 Hz, 1H), 3.87 - 3.75 (m, 1H), 3.59 (s, 3H), 2.88 (dd, J = 10.9, 13.3 Hz, 1H), 2.78 (dd, J = 10.8, 13.4 Hz, 1H), 2.69 (s, 3H), |
| Example 62 | | 2.65 (s, 3H), 2.59 (s, 6H), 1.35 (d, J = 6.1 |
| | | Hz, 3H). ¹⁹F NMR (377 MHz, CDCl₃) δ |
| | | −73.05 (s, 1F). |
| 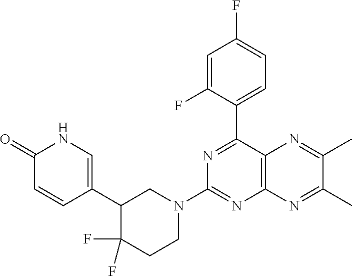 [SMILES: Cc1nc2nc(N3CCC(F)(F)C(c4ccc(=O)[nH]c4)C3)nc(-c3ccc(F)cc3F)c2nc1C] Example 189 | 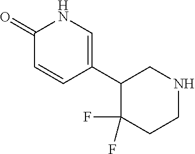 [SMILES: O=c1ccc(C2CNCCC2(F)F)c[nH]1] Int-C27 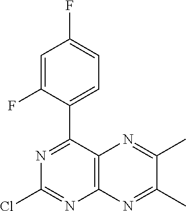 | Modification: The reaction was run in DMSO at 90 °C for 1 h. The crude mixture was purified by normal phase chromatography (50% to 100% EtOAc in hexanes followed by 0% to 2% MeOH in DCM) to afford 5-[1-[4-(2,4- difluorophenyl)-6,7-dimethyl-pteridin- 2-yl]-4,4-difluoro-3-piperidyl]-1H- pyridin-2-one (Example 189, 50 mg, 0.10 mmol, 55% yield) as a solid. ESI- MS: [M + H]⁺ = 485.4. ¹H NMR (CDCl₃, 400 MHz): δ 7.67 (1H, q, J = 7.5 Hz), 7.44 (1H, d, J = 9.5 Hz), 7.34 (1H, s), 6.99 (1H, t, J = 7.8 Hz), 6.92 (1H, t, J = 9.4 Hz), 6.53 (1H, d, J = 9.3 Hz), 5.15 (2H, br s), 3.30-3.40 (2H, m), 2.97 (1H, br s), 2.67 (3H, s), 2.55 (3H, s), 2.23 (1H, br s), 1.98-2.06 (1H, m). ¹⁹F NMR (CDCl₃, 376 MHz): δ −96.0 (1F, d, J = 248.6 Hz), −106.6 (2F, s), −113.7 (1F,d, J = 237.8 Hz) |

### Method 2: Synthesis of Examples 23 and 21

### Example 23:5-[(2S,6R)-4-[6,7-dimethyl-4-[2-(trifluoromethyl) thiazol-5-yl]pteridin-2-yl]-6-methyl-morpholin-2-yl]-1-methyl-pyridin-2-one

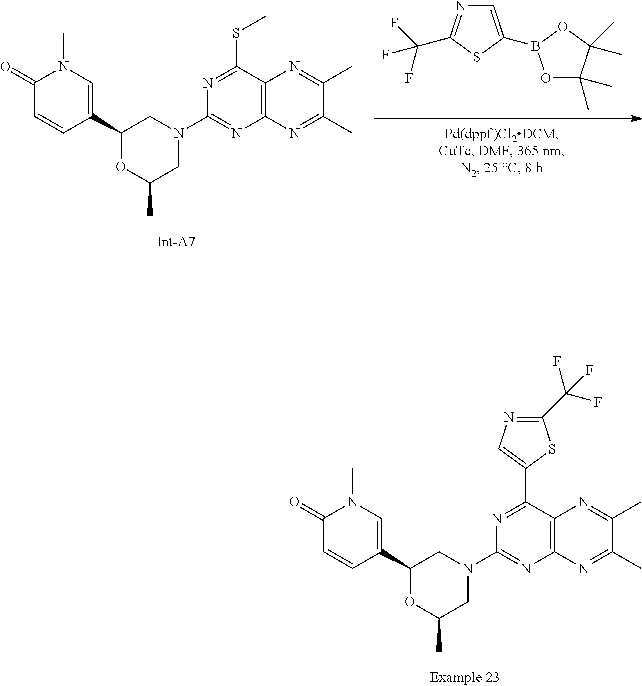

A solution of 5-[(2S,6R)-4-(6,7-dimethyl-4-methylsulfanyl-pteridin-2-yl)-6-methyl-morpholin-2-yl]-1-methyl-pyridin-2-one (Int-A7, 1 equiv., 55 mg, 0.09 mmol), 5-(4,4,5,5-tetramethyl-1,3,2-dioxaborolan-2-yl)-2-(trifluoromethyl) thiazole (3 equiv., 78 mg, 0.28 mmol), CuTc (2.2 equiv., 39 mg, 0.21 mmol) and Pd(dppf)Cl₂·DCM (0.3 equiv., 20 mg, 0.028 mmol) in dry DMF (2 mL), was flushed with argon for 3 min, and the suspension was irradiated (365 nm) under argon for 8 h. LCMS showed 20% of desired product. The mixture was diluted with water (50 mL) and extracted with EtOAc (50 mL×3). The combined organic layers were washed with brine, dried over Na₂SO₄, filtered, and concentrated under reduced pressure to give a residue. The residue was purified by prep-HPLC (Condition 3, Gradient a) to afford the 5-[(2S,6R)-4-[6,7-dimethyl-4-[2-(trifluoromethyl) thiazol-5-yl]pteridin-2-yl]-6-methyl-morpholin-2-yl]-1-methyl-pyridin-2-one (Example 23, 4.7 mg, 0.009 mmol, 10% yield) as a solid. LCMS: [M+H]⁺=518.4; purity=100% (220 nm); Ret. time=0.591 min. 1H NMR (400 MHz, CDCl₃) δ 1.29 (br d, J=5.0 Hz, 3H), 2.78 (br d, J=3.0 Hz, 6H), 2.85-2.94 (m, 1H), 2.95-3.05 (m, 1H), 3.63 (s, 3H), 3.82-3.91 (m, 1H), 4.40-4.48 (m, 1H), 4.84-5.19 (m, 2H), 6.59-6.75 (m, 1H), 7.44-7.56 (m, 2H), 9.36 (s, 1H),

An analogous procedure was followed to obtain the following compound.

| Structure | Intermediate | Characterization |
|---|---|---|
| 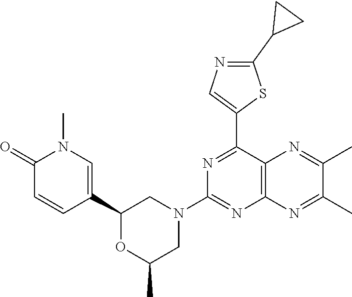 [SMILES: Cc1nc2nc(N3C[C@@H](C)O[C@@H](c4ccc(=O)n(C)c4)C3)nc(-c3cnc(C4CC4)s3)c2nc1C] | 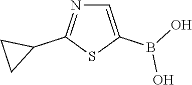 [SMILES: OB(O)c1cnc(C2CC2)s1] | The residue was purified by prep-HPLC (Condition 3, Gradient b) to afford 5- [(2S,6R)-4-[4-(2-cyclopropylthiazol-5- yl)-6,7-dimethyl-pteridin-2-yl]-6- methyl-morpholin-2-yl]-1-methyl- pyridin-2-one (Example 21, 14 mg, 0.026 mmol, 43% yield) as a sollid. LCMS: [M + H]⁺ = 490.1; purity = 92% (200 nm): Ret. time = 0.546 min.¹H NMR (400 MHz, CDCl₃) δ 1.26 (br d, J = 5.4 Hz, 4H), 1.37 (br d, J = 5.9 Hz, 3H), 2.37-2.50 (m, 1H), 2.72 (d, J = 5.1 Hz, 6H), 2.83 (br dd, J = 13.1, 11.2 Hz, 1H), 2.88-2.97 (m, 1H), 3.60 (s, 3H), 3.78-3.88 (m, 1H), 4.35-4.44 (m, 1H), 4.86-5.13 (m, 2H), 6.64 (br d, J = 8.3 Hz, 1H), 7.40-7.51 (m, 2H), 9.19 (s, 1H). |
| Example 21 | | |

### Method 3: Synthesis of Examples 1-6, 11-15, 18-20, 25-26, 31-32, 64-65, 67-79, 123-125, 155- and 160

### Example 3:5-(4-(4-(2,4-difluorophenyl)-6,7-dimethylpteridin-2-yl)tetrahydro-2H-pyran-2-yl)-1-methylpyridin-2 (1H)-one

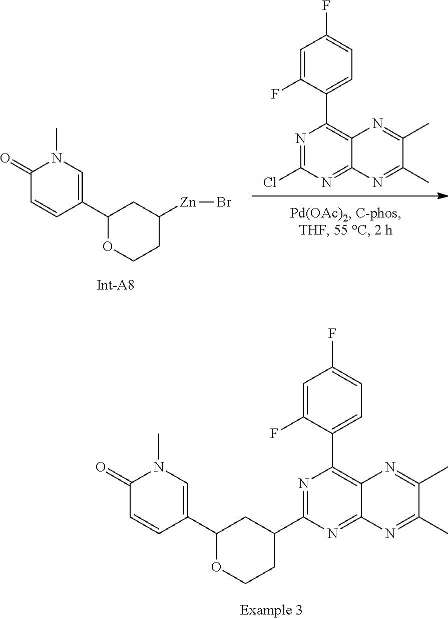

To a suspension of C-phos (0.1 equiv., 85 mg, 0.196 mmol), Pd(OAc)₂ (0.05 equiv., 22 mg, 0.098 mmol) and 2-chloro-4-(2,4-difluorophenyl)-6,7-dimethyl-pteridine (1 equiv., 600 mg, 1.96 mmol) in THF (6 mL), which was purged with N₂ for 4 times, was added bromo-[2-(1-methyl-6-oxo-3-pyridyl)tetrahydropyran-4-yl]zinc (Int-A8, 1.2 equiv., 792 mg, 2.4 mmol). The resulting mixture was stirred at 55° C. for 2 hours. LCMS showed reactants were consumed and the product was delivered with 33% of desired mass. The reaction mixture was poured into H₂O (12 mL), extracted with EtOAc (15 mL×3), dried over Na₂SO₄, filtered, and concentrated under reduced pressure to give the crude, which was then purified with silica gel column chromatography (9% MeOH in EtOAc, R_{f}=0.3) and concentrated under reduced pressure to give 400 mg 5-(4-(4-(2,4-difluorophenyl)-6,7-dimethylpteridin-2-yl)tetrahydro-2H-pyran-2-yl)-1-methylpyridin-2 (1H)-one (Example 3). The product was further purified with Prep-HPLC (FA) and lyophilized to give 1-methyl-5-[rac-(2R,4R)-4-[4-(2,4-difluorophenyl)-6,7-dimethyl-pteridin-2-yl]tetrahydropyran-2-yl]pyridin-2-one (100 mg, 0.216 mmol, 11% yield) and 1-methyl-5-[rac-(2R,4S)-4-[4-(2,4-difluorophenyl)-6,7-dimethyl-pteridin-2-yl]tetrahydropyran-2-yl]pyridin-2-one (140 mg, 0.302 mmol, 15% yield) as a solid.

LCMS (1-methyl-5-[rac-(2R,4R)-4-[4-(2,4-difluorophenyl)-6,7-dimethyl-pteridin-2-yl]tetrahydropyran-2-yl]pyridin-2-one): [M+H]⁺=464.3; purity=99% (220 nm); Ret. time=0.491 min

LCMS (1-methyl-5-[rac-(2R,4S)-4-[4-(2,4-difluorophenyl)-6,7-dimethyl-pteridin-2-yl]tetrahydropyran-2-yl]pyridin-2-one): [M+H]⁺=464.3; purity=99% (220 nm); Ret. time=0.498 min

### Examples 1 and 2

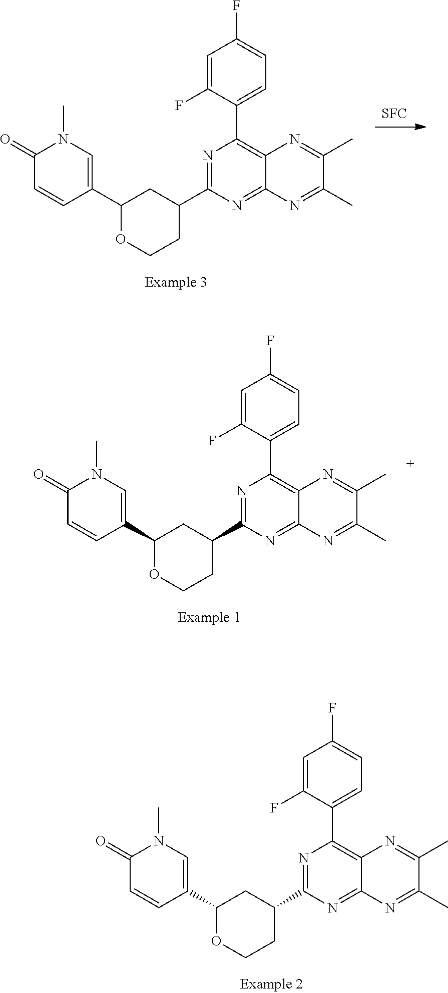

The racemate was purified by SFC (Condition 1, Gradient a) and prep-TLC (9% MeOH in DCM, R_{f}=0.4) to afford 1-methyl-5-[(2R,4S)-4-[4-(2,4-difluorophenyl)-6,7-dimethyl-pteridin-2-yl]tetrahydropyran-2-yl]pyridin-2-one (Example 1, 20 mg, 0.04 mmol, 38% yield) as a solid, and 5-[(2S,4R)-4-[4-(2,4-difluorophenyl)-6,7-dimethyl-pteridin-2-yl]tetrahydropyran-2-yl]-1-methyl-pyridin-2-one (Example 2, 8.7 mg, 0.02 mmol, 17% yield) as a solid.

Example 1: LCMS: [M+H]⁺=464.1; purity=94% (220 nm); Ret. time=0.501 min. ¹H NMR (400 MHz, CDCl₃) δ 7.79-7.71 (m, 1H), 7.45-7.34 (m, 2H), 7.13-6.97 (m, 2H), 6.56 (br d, J=10.0 Hz, 1H), 4.38-4.25 (m, 2H), 3.80 (dt, J=3.1, 11.4 Hz, 1H), 3.64-3.47 (m, 4H), 2.84 (s, 3H), 2.73 (s, 3H), 2.45-2.31 (m, 1H), 2.24-2.05 (m, 3H).

Example 2: LCMS: [M+H]⁺=464.1; purity=98% (220 nm); Ret. time=0.500 min. ¹H NMR (400 MHz, CDCl₃) δ 7.80-7.70 (m, 1H), 7.47-7.37 (m, 2H), 7.09 (br t, J=8.2 Hz, 1H), 7.04-6.96 (m, 1H), 6.58 (d, J=9.5 Hz, 1H), 4.41-4.26 (m, 2H), 3.86-3.74 (m, 1H), 3.55 (s, 3H), 2.85 (s, 3H), 2.73 (s, 3H), 2.38 (br d, J=13.3 Hz, 1H), 2.23-2.07 (m, 3H).

An analogous procedure was followed to obtain the following compounds.

| Structure | Starting Material | Characterization |
|---|---|---|
| 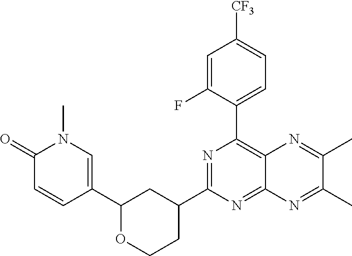 [SMILES: Cc1nc2nc(C3CCOC(c4ccc(=O)n(C)c4)C3)nc(-c3ccc(C(F)(F)F)cc3F)c2nc1C] | 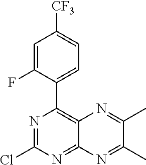 | The solid was purified with Prep-HPLC (FA) and lyophilized to give ~140 mg racemate product Example 6, which was then purified by SFC and lyophilized to give 1-methyl-5-[(2R,4S)-4-[4-[2- fluoro-4-(trifluoro-methyl)phenyl]-6,7- dimethyl-pteridin-2-yl]tetrahydropyran- 2-yl]pyridin-2-one (Example 4, 33 mg, 0.06 mmol, 4% yield) as a solid and 1- methyl-5-[(2S,4R)-4-[4-[2-fluoro-4- (trifluoromethyl)phenyl]-6,7-dimethyl- pteridin-2-yl]tetrahydropyran-2- yl]pyridin-2-one (Example 5, 56 mg, 0.11 mmol, 6% yield) as a solid. Example 4: LCMS: [M + H]⁺ = 514.3; purity = 98% (220 nm); Ret. time = 0.535 |
| Example 6 | | min. ¹H NMR (400 MHz, CDCl₃) δ 7.86 |
| Then, SFC - | | (t, J = 7.1 Hz, 1H), 7.67-7.61 (m, 1H), |
| 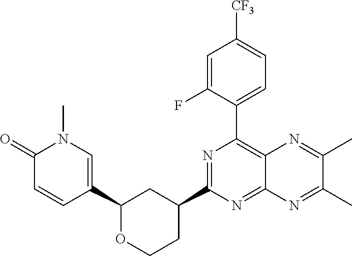 [SMILES: Cc1nc2nc([C@H]3CCO[C@@H](c4ccc(=O)n(C)c4)C3)nc(-c3ccc(C(F)(F)F)cc3F)c2nc1C] | | 7.57-7.51 (m, 1H), 7.43-7.37 (m, 2H), 6.60-6.53 (m, 1H), 4.41-4.25 (m, 2H), 3.81 (dt, J = 3.5, 11.5 Hz, 1H), 3.56 (s, 4H), 2.87 (s, 3H), 2.74 (s, 3H), 2.43- 2.36 (m, 1H), 2.28-2.04 (m, 3H). Example 5: LCMS: [M + H]⁺ = 514.3; purity = 98% (220 nm); Ret. time = 0.535 min. ¹H NMR (400 MHz, CDCl₃) δ 7.86 (t, J = 7.3 Hz, 1H), 7.64 (d, J = 8.0 Hz, 1H), 7.54 (d, J = 8.9 Hz, 1H), 7.44-7.37 (m, 2H), 6.60-6.54 (m, 1H), 4.39-4.28 (m, 2H), 3.81 (dt, J = 3.1, 11.5 Hz, 1H), 3.62-3.53 (m, 4H), 2.87 (s, 3H), 2.74 (s, 3H), 2.39 (br d, J = 13.4 Hz, 1H), 2.29- 2.07 (m, 3H). |
| Example 4 | | |
| 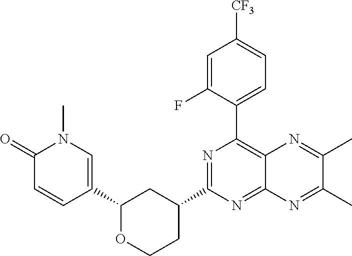 [SMILES: Cc1nc2nc([C@@H]3CCO[C@H](c4ccc(=O)n(C)c4)C3)nc(-c3ccc(C(F)(F)F)cc3F)c2nc1C] | | |
| Example 5 | | |
| 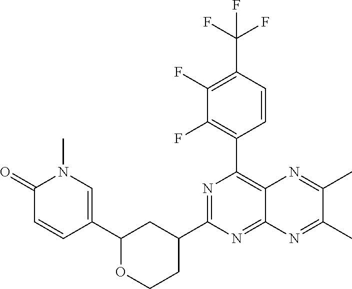 [SMILES: Cc1nc2nc(C3CCOC(c4ccc(=O)n(C)c4)C3)nc(-c3ccc(C(F)(F)F)c(F)c3F)c2nc1C] | 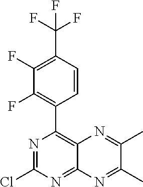 [SMILES: Cc1nc2nc(Cl)nc(-c3ccc(C(F)(F)F)c(F)c3F)c2nc1C] Int-A11 | The crude (Example 20) was purified by silica gel column chromatography (33% EtOAc in PE, R_{f} = 0.3) to remove the color and then purified by prep-HPLC (FA) and freeze-drying to give 90 mg racemic product. The 90 mg racemic product was combined with another batch and purified by SFC (Condition 2, Gradient a) to give 5-[(2R,4S)-4-[4-[2,3- difluoro-4-(trifluoromethyl)-phenyl]- 6,7-dimethyl-pteridin-2- yl]tetrahydropyran-2-yl]-1-methyl- pyridin-2-one (Example 18, 7.2 mg, 0.014 mmol, 2.5% yield) as a solid and 5-[(2S,4R)-4-[4-[2,3-difluoro-4- (trifluoro-methyl)phenyl]-6,7-dimethyl- pteridin-2-yl\|tetrahydropyran-2-yl]-1- methyl-pyridin-2-one (Example 19, 8.3 |
| Example 20 | | mg, 0.016 mmol, 3% yield) as a solid. |
| Then, SFC- | | Example 18: LCMS: Ret. Time = 0.632 |
| 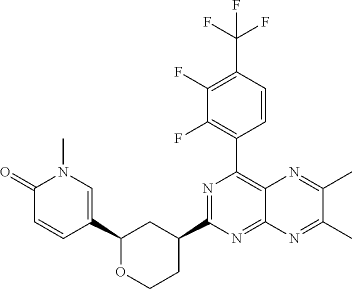 [SMILES: Cc1nc2nc([C@H]3CCO[C@@H](c4ccc(=O)n(C)c4)C3)nc(-c3ccc(C(F)(F)F)c(F)c3F)c2nc1C] | | min; [M + H]⁺ = 532.4, purity = 100%, (220 nm), Ret. Time = 0.559 min. ¹H NMR (400 MHz, CDCl₃) δ: 2.04-2.12 (m, 1H), 2.14-2.25 (m, 1H), 2.21 (br d, J = 3.5 Hz, 1H), 2.33-2.43 (m, 1H), 2.76 (s, 3H), 2.87 (s, 3H), 3.52-3.60 (m, 1H), 3.56 (s, 2H), 3.81 (td, J = 11.60, 2.9 Hz, 1H), 4.26-4.39 (m, 2H), 6.55-6.61 (m, 1H), 7.37-7.44 (m, 2H), 7.59 (t, J = 2.9 Hz, 2H). Example 19: LCMS: Ret. Time = 1.253 min; [M + H]⁺ = 532.4, purity = 100%, (220 nm), Ret. Time = 0.552 min. ¹H NMR (400 MHz, CDCl₃) δ: 2.04-2.25 (m, 3H), 2.32-2.44 (m, 1H), 2.76 (s, 3H), 2.87 (s, 3H), 3.52-3.64 (m, 4H), 3.73-3.88 (m, 1H), 4.25-4.41 (m, 2H), 6.58 (br d, J = 10.1 Hz, 1H), 7.36-7.45 |
| Example 18 | | (m, 2H), 7.54-7.64 (m, 2H). |
| 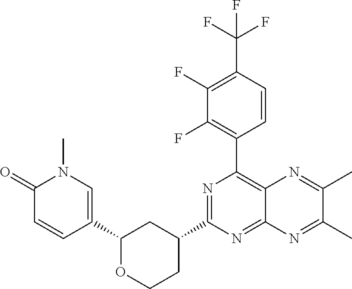 [SMILES: Cc1nc2nc([C@@H]3CCO[C@H](c4ccc(=O)n(C)c4)C3)nc(-c3ccc(C(F)(F)F)c(F)c3F)c2nc1C] | | |
| Example 19 | | |
| 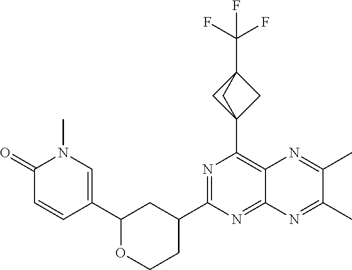 | 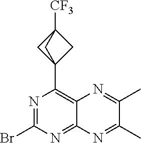 Int-A14 | The residue was purified by silica gel column chromatography (100% to 66% EtOAc in MeOH) (TLC: 9% MeOH in DCM, R_{f} = 0.5) to give 270 mg of the crude product, which was purified by prep-TLC (6% MeOH in DCM; the desired product R_{f} = 0.3) to give 30 mg the desired product (86% purity, checked by LCMS). 30 mg the desired product was purified by reversed-phase chromatography (40-60% [(0.1% FA)H₂O—MeCN) and lyophilized to give 5-(4-(6,7-dimethyl-4-(3- (trifluoromethyl)-bicyclo[1.1.1] pentan- 1-yl)pteridin-2-yl)tetrahydro-2H-pyran- 2-yl)-1-methylpyridin-2(1H)-one (Example |
| Example 27 | | 27, 15 mg, 0.031 mmol, 6% yield) as a |
| Then, SFC - | | solid. LCMS: [M + H]⁺ = 486.4; purity = |
| 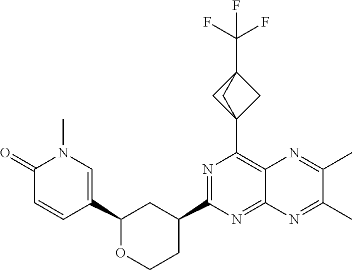 [SMILES: Cc1nc2nc([C@H]3CCO[C@@H](c4ccc(=O)n(C)c4)C3)nc(C34CC(C(F)(F)F)(C3)C4)c2nc1C] | | 97% (220 nm); Ret. time = 0.563 min. ¹H NMR (400 MHz, CDCl₃) δ 7.49-7.34 (m, 2H), 6.70-6.58 (m, 1H), 4.61-4.25 (m, 2H), 3.88-3.76 (m, 1H), 3.57 (s, 3H), 3.51-3.40 (m, 1H), 2.84-2.78 (m, 6H), 2.68-2.63 (m, 6H), 2.32 (br d, J = 13.3 Hz, 1H), 2.20-2.06 (m, 2H), 2.05- 2.01 (m, 1H). Example 27 (1 equiv., 13 mg, 0.027 mmol) was purified by prep-SFC to give 10 mg cis product (86% purity) and 3 mg trans product (60% purity). 10 mg cis product was purified by prep-TLC (6% MeOH in DCM, R_{f} = 0.6) and lyophilized to give cis-5-(4-(6,7- dimethyl-4-(3-(trifluoromethyl) bicyclo[1.1.1]-pentan-1-yl)pteridin-2- |
| Example 25 | | yl)tetrahydro-2H-pyran-2-yl)-1- |
| 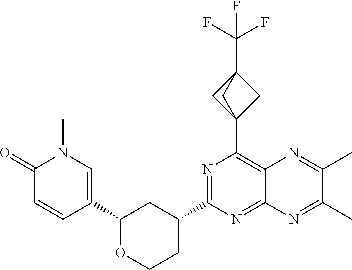 [SMILES: Cc1nc2nc([C@@H]3CCO[C@H](c4ccc(=O)n(C)c4)C3)nc(C34CC(C(F)(F)F)(C3)C4)c2nc1C] | | methylpyridin-2(1H)-one (Example 27a, 4.4 mg, 0.008 mmol, 31% yield) as a solid. LCMS: [M + H]⁺ = 486.4; purity = 92% (220 nm); Ret. time = 0.558 min. ¹H NMR (400 MHz, CDCl₃) δ 7.48-7.34 (m, 2H), 6.59 (d, J = 10.3 Hz, 1H), 4.42- 4.23 (m, 2H), 3.80 (dt, J = 3.3, 11.5 Hz, 1H), 3.56 (s, 3H), 3.46 (tt, J = 4.3, 11.6 Hz, 1H), 2.80 (d, J = 14.4 Hz, 6H), 2.65 (s, 6H), 2.32 (br d, J = 13.1 Hz, 1H), 2.20- 2.09 (m, 2H), 2.07-2.01 (m, 1H). Cis-5-[4-[6,7-dimethyl-4-[3- (trifluoromethyl)-1-bicyclo[1.1.1]- pentanyl]pteridin-2-yl]-tetrahydropyran- 2-yl]-1-methyl-pyridin-2-one (Example 27a, 1 equiv., 140 mg, 0.29 mmol) was separated by prep-SFC (Condition 1, |
| Example 26 | | Gradient a-1) to afford 5-[(2R,4S)-4- |
| | | [6,7-dimethyl-4-[3-(trifluoromethyl)-1- |
| | | bicyclo[1.1.1]-pentanyl]pteridin-2-yl]- |
| | | tetrahydropyran-2-yl]-1-methyl-pyridin- |
| | | 2-one (Example 25, 28 mg, 0.055 mmol, |
| | | 19% yield) as a solid and 5-[(2S,4R)-4- |
| | | [6,7-dimethyl-4-[3-(trifluoromethyl)-1- |
| | | bicyclo[1.1.1]-pentanyl]pteridin-2- |
| | | yl]tetrahydropyran-2-yl]-1-methyl- |
| | | pyridin-2-one (Example 26, 38 mg, |
| | | 0.072 mmol, 25% yield) as a solid. |
| | | Example 25: LCMS: [M + H]⁺ = 486.2; |
| | | purity = 96% (220 nm); Ret. time = 0.561 |
| | | min. ¹H NMR (400 MHz, CDCl₃) δ 7.44- |
| | | 7.36 (m, 2H), 6.57 (d, J = 10.0 Hz, 1H), |
| | | 4.38-4.24 (m, 2H), 3.79 (dt, J = 2.8, 11.4 |
| | | Hz, 1H), 3.55 (s, 3H), 3.50-3.40 (m, |
| | | 1H), 2.80 (d, J = 14.4 Hz, 6H), 2.65 (s, |
| | | 6H), 2.31 (br d, J = 13.3 Hz, 1H), 2.19- |
| | | 1.97 (m, 3H). |
| | | Example 26: LCMS: [M + H]⁺ = 486.2; |
| | | purity = 92% (220 nm); Ret. time = 0.563 |
| | | min. ¹H NMR (400 MHz, CDCl₃) δ 7.44- |
| | | 7.36 (m, 2H), 6.58 (br d, J = 10.0 Hz, |
| | | 1H), 4.39-4.24 (m, 2H), 3.80 (dt, J = 3.1, |
| | | 11.4 Hz, 1H), 3.56 (s, 3H), 3.49 (br s, |
| | | 1H), 2.80 (d, J = 14.4 Hz, 6H), 2.65 (s, |
| | | 6H), 2.32 (br d, J = 13.0 Hz, 1H), 2.18- |
| | | 1.96 (m, 3H). |
| 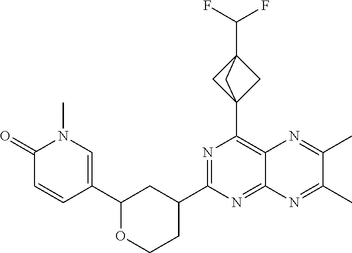 [SMILES: Cc1nc2nc(C3CCOC(c4ccc(=O)n(C)c4)C3)nc(C34CC(C(F)F)(C3)C4)c2nc1C] | 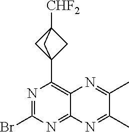 Int-A15 | The residue was purified by silica gel flash chromatography (0-30% EtOAc in MeOH) to give the crude product, which was purified by prep-TLC (5% MeOH in DCM, R_{f} = 0.3) to give 5-[4-[4-[3- (difluoromethyl)-1-bicyclo- [1.1.1]pentanyl]-6,7-dimethyl-pteridin- 2-yl]tetrahydropyran-2-yl]-1-methyl- pyridin-2-one (cis-Example 33, 100 mg, 0.214 mmol, 76% yield) as a solid. LCMS: Ret. time = 0.537 min, 468.2 = [M + H]⁺, ESI⁺. 5-[4-[4-[3-(difluoromethyl)-1- bicyclo[1.1.1]-pentanyl]-6,7-dimethyl- pteridin-2-yl]-tetrahydropyran-2-yl]-1- methyl-pyridin-2-one (Example 33, 1 |
| Example 33 | | equiv., 100 mg, 0.214 mmol) was |
| 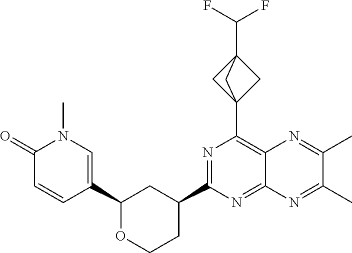 [SMILES: Cc1nc2nc([C@H]3CCO[C@@H](c4ccc(=O)n(C)c4)C3)nc(C34CC(C(F)F)(C3)C4)c2nc1C] | | purified by SFC (Condition 4, Gradient a) to give 5-[(2R,4S)-4-[4-[3- (difluoromethyl)-1-bicyclo[1.1.1]- pentanyl]-6,7-dimethyl-pteridin-2-yl]- tetrahydropyran-2-yl]-1-methyl-pyridin- 2-one (Example 31, 24 mg, 0.050 mmol, 23% yield) as a solid. The crude product was purified by second SFC (Condition 5, Gradient a) to give 5-[(2S,4R)-4-[4-[3- (difluoromethyl)-1-bicyclo[1.1.1]- pentanyl]-6,7-dimethyl-pteridin-2- yl]tetrahydropyran-2-yl]-1-methyl- pyridin-2-one (Example 32, 9.5 mg, 0.02 mmol, 9% yield) as a solid. Example 31: LCMS: [M + H]⁺ = 468.2; purity = 95% (220 nm); Ret. time = 0.530 |
| Example 31 | | min. ¹H NMR (400 MHz, CDCl₃) δ 1.98- |
| 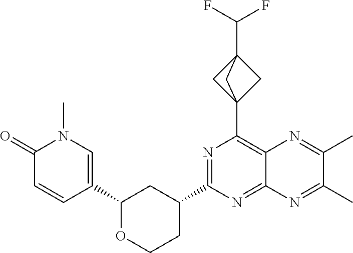 [SMILES: Cc1nc2nc([C@@H]3CCO[C@H](c4ccc(=O)n(C)c4)C3)nc(C34CC(C(F)F)(C3)C4)c2nc1C] | | 2.08 (m, 1H), 2.09-2.19 (m, 2H), 2.32 (br d, J = 13.4 Hz, 1H), 2.55 (s, 6H), 2.79 (d, J = 14.3 Hz, 6H), 3.39-3.50 (m, 1H), 3.56 (s, 3H), 3.80 (td, J = 11.4, 3.6 Hz, 1H), 4.23-4.38 (m, 2H), 5.65-6.05 (m, 1H), 6.53-6.60 (m, 1H), 7.36-7.44 (m, 2H). Example 32: LCMS: [M + H]⁺ = 468.2; purity = 97% (220 nm); Ret. time = 0.530 min. ¹H NMR (400 MHz, CDCl₃) δ 1.96- 2.17 (m, 3H), 2.32 (br d, J = 13.1 Hz, 1H), 2.55 (s, 6H), 2.79 (d, J = 14.1 Hz, 6H), 3.39-3.50 (m, 1H), 3.56 (s, 3H), 3.80 (td, J = 11.5, 3.3 Hz, 1H), 4.25-4.37 (m, 2H), 5.70-6.02 (m, 1H), 6.54-6.61 (m, 1H), 7.37-7.44 (m, 2H). |
| Example 32 | | |
| 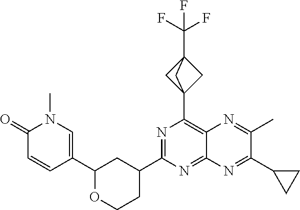 [SMILES: Cc1nc2c(C34CC(C(F)(F)F)(C3)C4)nc(C3CCOC(c4ccc(=O)n(C)c4)C3)nc2nc1C1CC1] | 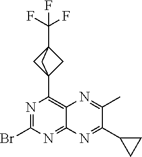 [SMILES: Cc1nc2c(C34CC(C(F)(F)F)(C3)C4)nc(Br)nc2nc1C1CC1] | First purification: The crude product 5-[4-[7-cyclopropyl- 6-methyl-4-[3-(trifluoro-methyl)-1- bicyclo[1.1.1]-pentanyl] pteridin-2- yl]tetrahydropyran-2-yl]-1-methyl- pyridin-2-one (Example 157, 1 equiv., 1.0 g, 1.95 mmol) was first purified by reversed-phase HPLC (35-45% [water (0.05% FA)-ACN], in 5 min) to afford crude product (130 mg) as an oil. Second purification: The crude product (130 mg) was purified by SFC (Condition 6, Gradient a) to give |
| Example 157 | | Crude A & Crude B. |
| Then, SFC - | | Third purification: |
| 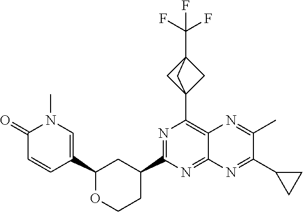 [SMILES: Cc1nc2c(C34CC(C(F)(F)F)(C3)C4)nc([C@H]3CCO[C@@H](c4ccc(=O)n(C)c4)C3)nc2nc1C1CC1] | | Crude A was further purified by prep- HPLC (Condition 5, Gradient a) to afford 1-methyl-5-[(2S,4R)-4-[7-cyclopropyl- 6-methyl-4-[3-(trifluoro-methyl)-1- bicyclo[1.1.1]pentanyl]pteridin-2- yl]tetrahydropyran-2-yl]pyridin-2-one (Example 156, 4.4 mg, 0.008 mmol, 0.43% yield) as a solid. LCMS: [M + H]⁺ = 512.2; purity = 99% (220 nm); Ret. time = 0.598 min. ¹H NMR (400 MHz, CDCl₃, 301 K) δ 7.45-7.37 (m, 2H), 6.59 (d, J = 10.1 Hz, 1H), 4.38-4.25 (m, 2H), 3.80 (dt, J = 3.4, 11.4 Hz, 1H), 3.57 |
| Example 155 | | (s, 3H), 3.50-3.40 (m, 1H), 2.97 (s, 3H), |
| 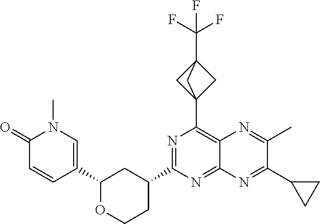 [SMILES: Cc1nc2c(C34CC(C(F)(F)F)(C3)C4)nc([C@@H]3CCO[C@H](c4ccc(=O)n(C)c4)C3)nc2nc1C1CC1] | | 2.61 (s, 6H), 2.43-2.35 (m, 1H), 2.31 (br d, J = 13.1 Hz, 1H), 2.18-2.08 (m, 2H), 2.07-1.97 (m, 1H), 1.31-1.24 (m, 4H). Crude B was further purified by prep- HPLC (Condition 5, Gradient b) to afford 1-methyl-5-[(2R,4S)-4-[7- cyclopropyl-6-methyl-4-[3-(trifluoro- methyl)-1-bicyclo[1.1.1]pentanyl]- pteridin-2-yl]tetrahydropyran-2- yl]pyridin-2-one (Example 155, 18 mg, 0.034 mmol, 2% yield) as a solid. LCMS: [M + H]⁺ = 512.3; purity = 97% (220 nm); Ret. time = 0.595 min. ¹H NMR (400 MHz, CDCl₃, 301 K) δ 7.42- |
| Example 156 | | 7.38 (m, 2H), 6.59-6.55 (m, 1H), 4.35- |
| | | 4.25 (m, 2H), 3.79 (dt, J = 3.4, 11.4 Hz, |
| | | 1H), 3.55 (s, 3H), 3.48-3.39 (m, 1H), |
| | | 2.96 (s, 3H), 2.60 (s, 6H), 2.42-2.34 (m, |
| | | 1H), 2.30 (br d, J = 13.3 Hz, 1H), 2.15- |
| | | 1.96 (m, 3H), 1.30-1.24 (m, 4H). |
| 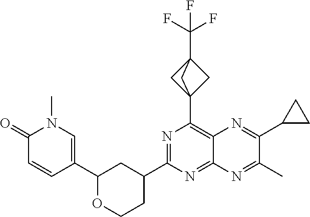 [SMILES: Cc1nc2nc(C3CCOC(c4ccc(=O)n(C)c4)C3)nc(C34CC(C(F)(F)F)(C3)C4)c2nc1C1CC1] | 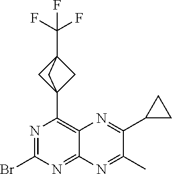 [SMILES: Cc1nc2nc(Br)nc(C34CC(C(F)(F)F)(C3)C4)c2nc1C1CC1] | First purification: The crude 5-[4-[6-cyclopropyl-7- methyl-4-[3-(trifluoromethyl)-1- bicyclo[1.1.1]pentanyl]pteridin-2- yl]tetrahydropyran-2-yl]-1-methyl- pyridin-2-one (Example 160, 1 equiv., 1.0 g, 1.95 mmol) was purified by reversed-phase HPLC (85-90% [water (0.05% FA)-ACN], in 5 min) to afford crude product (120 mg) as an oil. Second purification: The crude product (120 mg) was purified by SFC (Condition 7, Gradient a) to give Crude A (48 mg) and Crude B (52 mg). |
| Example 160 | | Third purification: |
| Then, SFC - | | Crude A was further purified by SFC |
| 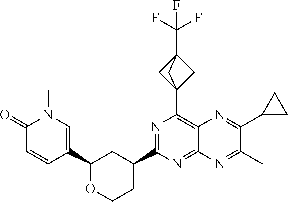 [SMILES: Cc1nc2nc([C@H]3CCO[C@@H](c4ccc(=O)n(C)c4)C3)nc(C34CC(C(F)(F)F)(C3)C4)c2nc1C1CC1] | | (Condition 8, Gradient a) to give Crude 1 and Crude 2. Crude B was further purified by SFC (Condition 6, Gradient b) to give Crude 3 and Crude 4. Fourth purification: Crude 1 was further purified by prep- HPLC (Condition 5, Gradient c) to afford 1-methyl-5-[(2S,4R)-4-[6-cyclopropyl- 7-methyl-4-[3-(trifluoro-methyl)-1- bicyclo[1.1.1]pentanyl]pteridin-2- yl]tetrahydropyran-2-yl]pyridin-2-one (Example 159, 3.3 mg, 0.006 mmol, 0.3% yield) as a solid. |
| Example 158 | | Crude 2 was further purified by prep- |
| 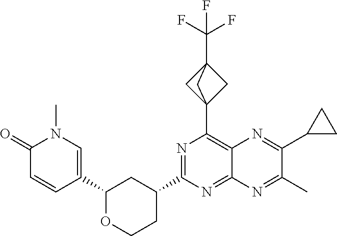 [SMILES: Cc1nc2nc([C@@H]3CCO[C@H](c4ccc(=O)n(C)c4)C3)nc(C34CC(C(F)(F)F)(C3)C4)c2nc1C1CC1] | | HPLC (Condition 5, Gradient c) to afford 1-methyl-5-[(2R,4S)-4-[6-cyclopropyl- 7-methyl-4-[3-(trifluoro-methyl)-1- bicyclo[1.1.1]pentanyl]pteridin-2- yl]tetrahydropyran-2-yl]pyridin-2-one (Example 158, 4.4 mg, 0.009 mmol, 0.4% yield) as a solid. Crude 3 was further purified by prep- HPLC (Condition 5, Gradient c) to afford 1-methyl-5-[(2S,4R)-4-[6-cyclopropyl- 7-methyl-4-[3-(trifluoro-methyl)-1- bicyclo[1.1.1]pentanyl] pteridin-2- yl]tetrahydropyran-2-yl]pyridin-2-one (Example 159, 13 mg, 0.025 mmol, 1.3% |
| Example 159 | | yield) as a solid. LCMS: [M + H]⁺ = |
| | | 512.2; purity = 99% (220 nm); Ret. |
| | | time = 0.614 min. ¹H NMR (400 MHz, |
| | | CDCl₃, 301 K) δ 7.41-7.36 (m, 2H), |
| | | 6.60-6.55 (m, 1H), 4.34-4.24 (m, 2H), |
| | | 3.77 (dt, J = 2.4, 11.9 Hz, 1H), 3.55 (s, |
| | | 3H), 3.45 (tt, J = 3.7, 11.9 Hz, 1H), 2.90 |
| | | (s, 3H), 2.64 (s, 6H), 2.39-2.31 (m, 1H), |
| | | 2.27-2.20 (m, 1H), 2.19-2.10 (m, 1H), |
| | | 2.09-1.93 (m, 2H), 1.53-1.48 (m, 2H), |
| | | 1.30-1.24 (m, 2H) and ee = 100%. |
| | | Crude 4 was further purified by prep- |
| | | HPLC (Condition 5, Gradient c) to afford |
| | | 1-methyl-5-[(2R,4S)-4-[6-cyclopropyl- |
| | | 7-methyl-4-[3-(trifluoro-methyl)-1- |
| | | bicyclo[1.1.1 ]pentanyl] |
| | | pteridin-2- |
| | | yl]tetrahydropyran-2-yl]pyridin-2-one |
| | | (Example 158, 14 mg, 0.0267 mmol, |
| | | 1.5% yield) as a solid. LCMS: [M + |
| | | H]⁺ = 512.2; purity = 99.6% (220 nm); |
| | | Ret. time = 0.607 min. ¹H NMR (400 |
| | | MHz, CDCl₃, 301 K) δ 7.41-7.35 (m, |
| | | 2H), 6.60-6.55 (m, 1H), 4.35-4.24 (m, |
| | | 2H), 3.77 (dt, J = 2.3, 12.0 Hz, 1H), 3.55 |
| | | (s, 3H), 3.45 (tt, J = 3.7, |
| | | 11.9 Hz, 1H), 2.90 |
| | | (s, 3H), 2.64 (s, 6H), 2.38-2.31 (m, 1H), |
| | | 2.27-2.20 (m, 1H), 2.15 (dt, J = 4.5, 12.5 |
| | | Hz, 1H), 2.09-1.94 (m, 2H), 1.53-1.47 |
| | | (m, 2H), 1.30-1.24 (m, 2H). ee = 100%. |
| | | Example 159: LCMS: (M + H)⁺ = 512.2; |
| | | purity = 98% (220 nm); Ret. time = 0.610 |
| | | min. ¹H NMR (400 MHz, CDCl₃, 301 K) |
| | | δ 7.41-7.36 (m, 2H), 6.60-6.55 (m, |
| | | 1H), 4.34-4.25 (m, 2H), 3.77 (dt, J = 2.3, |
| | | 12.0 Hz, 1H), 3.55 (s, 3H), 3.45 (tt, J = |
| | | 3.9, 12.0 Hz, 1H), 2.91 (s, 3H), 2.64 (s, |
| | | 6H), 2.39-2.31 (m, 1H), 2.26-2.20 (m, |
| | | 1H), 2.19-2.11 (m, 1H), 2.08-1.94 (m, |
| | | 2H), 1.52-1.48 (m, 2H), 1.28-1.26 (m, |
| | | 2H) and ee. 100%. |
| | | Example 158: LCMS: (M + H)⁺ = 512.2; |
| | | purity = 99% (220 nm); Ret. time = 0.608 |
| | | min. ¹H NMR (400 MHz, CDCl₃, 301 K) |
| | | δ 7.43-7.37 (m, 2H), 6.63-6.56 (m, |
| | | 1H), 4.35-4.27 (m, 2H), 3.78 (dt, J = 2.3, |
| | | 12.0 Hz, 1H), 3.57 (s, 3H), 3.47 (tt, J = |
| | | 3.6, 11.9 Hz, 1H), 2.92 (s, 3H), 2.65 (s, |
| | | 6H), 2.40-2.33 (m, 1H), 2.28-2.21 (m, |
| | | 1H), 2.20-2.12 (m, 1H), 2.10-1.95 (m, |
| | | 2H), 1.54-1.49 (m, 2H), 1.31-1.26 (m, |
| | | 2H) and ee = 100%. |
| 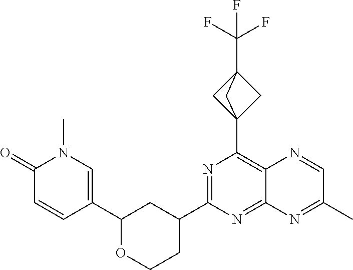 [SMILES: Cc1cnc2c(C34CC(C(F)(F)F)(C3)C4)nc(C3CCOC(c4ccc(=O)n(C)c4)C3)nc2n1] | 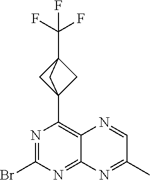 [SMILES: Cc1cnc2c(C34CC(C(F)(F)F)(C3)C4)nc(Br)nc2n1] Int-A50 | The crude product was purified by silica gel column chromatography (0-100% petroleum ether in EtOAc, and 100-50% EtOAc in MeOH) (TLC, 9% MeOH in DCM, desired product R_{f} = 0.4) to give 1-methyl-5-(4-(7-methyl-4-(3- (trifluoromethyl)bicyclo-[1.1.1]-pentan- 1-yl)pteridin-2-yl)tetrahydro-2H-pyran- 2-yl)pyridin-2(1H)-one (Example 125, 1 equiv., 150 mg, 0.32 mmol). Then, SFC - The crude was purified by SFC (Condition 5, Gradient a-1) to give Crude A (20 mg) and Crude B (10 mg). 10 mg Crude B was lyophilized to afford 1-methyl-5-((2R,4S)-4-(7-methyl-4-(3- (trifluoromethyl)bicyclo-[1.1.1]pentan- |
| Example 125 | | 1-yl)pteridin-2-yl)tetrahydro-2H-pyran- |
| Then, SFC - | | 2-yl)pyridin-2(1H)-one (Example 123, |
| 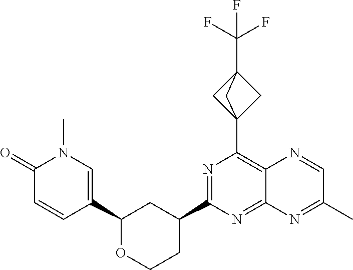 [SMILES: Cc1cnc2c(C34CC(C(F)(F)F)(C3)C4)nc([C@H]3CCO[C@@H](c4ccc(=O)n(C)c4)C3)nc2n1] | | 7.6 mg, 0.013 mmol, 4% yield) as a solid. LCMS: [M + H]⁺ = 472.2; purity = 80% (220 nm); Ret. time = 0.549 min. ¹H NMR (400 MHz, CDCl₃) δ 8.83-8.79 (m, 1H), 7.44-7.37 (m, 2H), 6.61-6.54 (m, 1H), 4.37-4.25 (m, 2H), 3.87 (s, 1H), 3.56 (s, 3H), 3.53-3.44 (m, 1H), 2.87 (s, 3H), 2.66 (s, 6H), 2.33 (br d, J = 13.0 Hz, 1H), 2.18-2.01 (m, 3H). 20 mg Crude A was purified by SFC (Condition 9, Gradient a) to give 1- methyl-5-[(2R,4R)-4-[7-methyl-4-[3- (trifluoromethyl)-1-bicyclo[1.1.1]- pentanyl]pteridin-2-yl]tetrahydropyran- 2-yl]pyridin-2-one (Example 343, 3.0 mg, 0.006 mmol, 2% yield) and 1- methyl-5-((2S,4R)-4-(7-methyl-4-(3- |
| Example 123 | | (trifluoromethyl)bicyclo[1.1.1]pentan-1- |
| 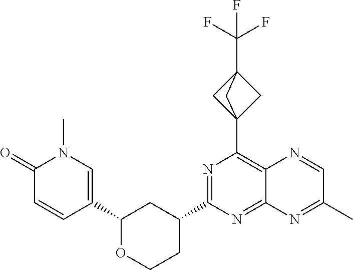 [SMILES: Cc1cnc2c(C34CC(C(F)(F)F)(C3)C4)nc([C@@H]3CCO[C@H](c4ccc(=O)n(C)c4)C3)nc2n1] | | yl)pteridin-2-yl)tetrahydro-2H-pyran-2- yl)pyridin-2(1H)-one (Example 124, 4.7 mg, 0.008 mmol, 3% yield) as a solid. LCMS: [M + H]⁺ = 472.2; purity = 80% (220 nm); Ret. time = 0.545 min. ¹H NMR (400 MHz, CDCl₃) δ 8.81 (s, 1H), 7.43-7.38 (m, 2H), 6.60-6.56 (m, 1H), 4.37-4.26 (m, 2H), 3.85-3.76 (m, 1H), 3.56 (s, 3H), 3.53-3.43 (m, 1H), 2.87 (s, 3H), 2.69-2.63 (m, 6H), 2.37-2.28 (m, 1H), 2.22-2.02 (m, 3H). |
| Example 124 | | |
| 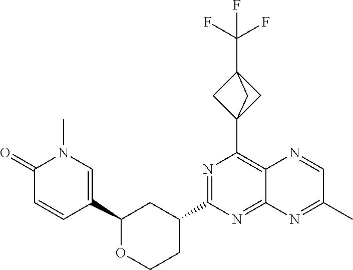 [SMILES: Cc1cnc2c(C34CC(C(F)(F)F)(C3)C4)nc([C@@H]3CCO[C@@H](c4ccc(=O)n(C)c4)C3)nc2n1] | | |
| Example 343 | | |
| 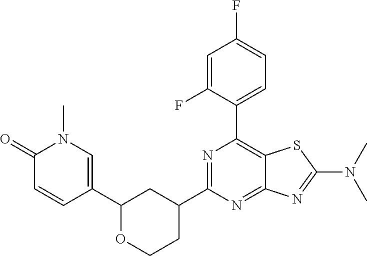 [SMILES: CN(C)c1nc2nc(C3CCOC(c4ccc(=O)n(C)c4)C3)nc(-c3ccc(F)cc3F)c2s1] | 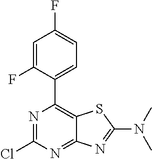 [SMILES: CN(C)c1nc2nc(Cl)nc(-c3ccc(F)cc3F)c2s1] | The crude (Example 15) was purified by silica gel column chromatography (9% MeOH in EtOAc, R_{f} = 0.3) and concentrated under reduced pressure to give 400 mg product, which was then purified with Prep-HPLC (Condition 6, Gradient a) and lyophilized to give 1- methyl-5-[rac-(2R,4S)-4-[7-(2,4- difluorophenyl)-2-(dimethylamino) thiazolo[4,5-c]pyrimidin-5- yl]tetrahydropyran-2-yl]pyridin-2-one (Example 15a, 150 mg, 0.31 mmol, 17% yield) as a solid and 1-methyl-5-[rac- (2R,4R)-4-[7-(2,4-difluorophenyl)-2- (dimethylamino)thiazolo[4,5- d]pyrimidin-5-yl]tetrahydropyran-2- |
| Example 15 | | yl]pyridin-2-one (Example 15b, 110 mg, |
| Then, SFC - | | 0.23 mmol, 12% yield) as a solid. |
| 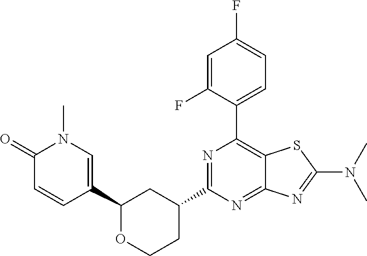 [SMILES: CN(C)c1nc2nc([C@@H]3CCO[C@@H](c4ccc(=O)n(C)c4)C3)nc(-c3ccc(F)cc3F)c2s1] | | Example 15a: LCMS: [M + H]⁺ = 484.3; purity = 100% (200 nm); Ret. time = 0.486 min. Example 15b: LCMS: [M + H]⁺ = 484.3; purity = 100% (200 nm); Ret. time = 0.492 min. Then, SFC - 1-methyl-5-[rac-(2R,4S)-4-[7-(2,4- difluorophenyl)-2-(dimethylamino)- thiazolo[4,5-d]pyrimidin-5- yl]tetrahydropyran-2-yl]pyridin-2-one (Example 15a, 150 mg) was purified with SFC (Condition 10, Gradient a) to give Crude A (60 mg) and Crude B (60 mg). The crude A was separated by SFC (Condition 10, Gradient a) and |
| Example 11 | | lyophilized to give 5-[(2R,4S)-4-[7-(2,4- |
| | | difluorophenyl)-2- |
| 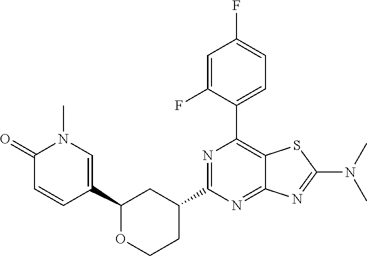 [SMILES: CN(C)c1nc2nc([C@@H]3CCO[C@@H](c4ccc(=O)n(C)c4)C3)nc(-c3ccc(F)cc3F)c2s1] | | (dimethylamino)thiazolo[4,5- d]pyrimidin-5-yl]tetrahydropyran-2-yl]- 1-methyl-pyridin-2-one (Example 13, 54 mg, 0.11 mmol, 90% yield) as a solid. LCMS: [M + H]+ = 484.2, purity = 100% (220 nm): Ret. time = 0.480 min. 1H NMR (400 MHz, CDCl₃) δ 7.79 (dt, J = 6.6, 8.4 Hz, 1H), 7.43-7.35 (m, 2H), 7.06 (dt, J = 2.4, 8.3 Hz, 1H), 6.98 (ddd, J = 2.5, 8.7, 10.7 Hz, 1H), 6.56 (d, J = 9.1 Hz, 1H), 4.32-4.23 (m, 2H), 3.75 (dt, J = 3.1, 11.5 Hz, 1H), 3.55 (s, 3H), 3.33 (ddd, J = 3.9, 7.8, 15.4 Hz, 6H), 2.31-2.23 (m, 1H), 2.21-1.98 (m, 4H). The crude B was separated by SFC (Condition 10, Gradient |
| Example 12 | | a) and lyophilized to give 5- |
| 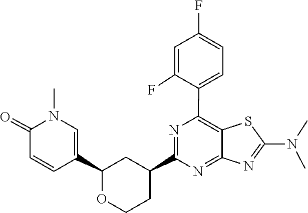 [SMILES: CN(C)c1nc2nc([C@H]3CCO[C@@H](c4ccc(=O)n(C)c4)C3)nc(-c3ccc(F)cc3F)c2s1] | | [(2S,4R)-4-[7-(2,4-difluorophenyl)-2- (dimethylamino)thiazolo[4,5- d]pyrimidin-5-yl]tetrahydropyran-2-yl]- 1-methyl-pyridin-2-one (Example 14, 46 mg, 0.09 mmol, 75% yield) as a solid. LCMS: [M + H]⁺ = 484.2, purity = 99% (200 nm); Ret. time = 0.482 min. ¹H NMR (400 MHz, CDCl₃) δ 7.79 (dt, J = 6.5, 8.4 Hz, 1H), 7.42-7.36 (m, 2H), 7.06 (dt, J = 2.1, 8.1 Hz, 1H), 6.98 (ddd, J = 2.4, 8.6, 10.7 Hz, 1H), 6.56 (d, J = 9.1 Hz, 1H), 4.28 (dt, J = 2.3, 10.7 Hz, 2H), 3.75 (dt, J = 3.0, 11.6 Hz, 1H), 3.55 (s, 3H), 3.42-3.24 (m, 6H), 2.30-2.22 (m, 1H), 2.21-1.98 (m, 4H). 1-methyl-5-[rac-(2R,4R)-4-[7-(2,4- |
| Example 13 | | difluorophenyl)-2-(dimethylamino) |
| 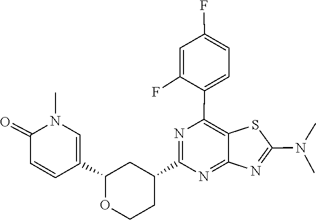 [SMILES: CN(C)c1nc2nc([C@@H]3CCO[C@H](c4ccc(=O)n(C)c4)C3)nc(-c3ccc(F)cc3F)c2s1] | | thiazolo[4,5-d]pyrimidin-5- yl]tetrahydropyran-2-yl]pyridin-2-one (Example 15b, 110 mg) was separated by SFC (Condition 10, Gradient a) and lyophilized to give 5-[(2S,4S)-4-[7-(2,4- difluorophenyl)-2- (dimethylamino)thiazolo[4,5- d]pyrimidin-5-yl]tetrahydropyran-2-yl]- 1-methyl-pyridin-2-one (Example 11, 46 mg, 0.09 mmol, 45% yield) as a solid and 5-[(2R,4R)-4-[7-(2,4-difluorophenyl)-2- (dimethylamino)thiazolo[4,5- d]pyrimidin-5-yl]tetrahydropyran-2-yl]- 1-methyl-pyridin-2-one (Example 12, 42 |
| Example 14 | | mg, 0.07 mmol, 37% yield) as a solid. |
| | | Example 11: LCMS: [M + H]⁺ = 484.2, |
| | | purity = 90% (220 nm); Ret. time = 0.494 |
| | | min. ¹H NMR (400 MHz, CDCl₃) δ 7.81 |
| | | (dt, J = 6.4, 8.5 Hz, 1H), 7.40 (dd, J = 2.4, |
| | | 9.3 Hz, 1H), 7.37 (s, 1H), 7.08 (dt, J = |
| | | 2.3, 8.2 Hz, 1H), 7.01 (ddd, J = 2.4, 8.6, |
| | | 10.7 Hz, 1H), 6.58 (d, J = 9.4 Hz, 1H), |
| | | 4.63 (dd, J = 2.3, 9.9 Hz, 1H), 3.97-3.86 |
| | | (m, 2H), 3.63-3.57 (m, 1H), 3.55 (s, |
| | | 3H), 3.36 (br d, J = 12.3 Hz, 6H), 2.72 |
| | | (br d, J = 13.5 Hz, 1H), 2.52 (br dd, J = |
| | | 1.8, 13.7 Hz, 1H), 2.19-2.11 (m, 1H), |
| | | 2.11-2.01 (m, 1H). |
| | | Example 12: LCMS: [M + H]⁺ = 484.2, |
| | | purity = 98% (200 nm); Ret. time = 0.494 |
| | | min. ¹H NMR (400 MHz, CDCl₃) δ 7.81 |
| | | (t, J = 6.4, 8.5 Hz, 1H), 7.41 (dd, J = 2.4, |
| | | 9.3 Hz, 1H), 7.37 (s, 1H), 7.08 (dt, J = |
| | | 2.1, 8.1 Hz, 1H), 7.01 (ddd, J = 2.4, 8.6, |
| | | 10.7 Hz, 1H), 6.58 (d, J = 9.4 Hz, 1H), |
| | | 4.63 (dd, J = 2.3, 10.0 Hz, 1H), 3.97- |
| | | 3.87 (m, 2H), 3.59 (br s, 1H), 3.56 (s, |
| | | 3H), 3.36 (br d, J = 12.4 Hz, 6H), 2.72 |
| | | (br d, J = 13.4 Hz, 1H), 2.52 (br dd, J = |
| | | 1.8, 13.7 Hz, 1H), 2.20-2.11 (m, 1H), |
| | | 2.10-2.01 (m, 1H). |

An analogous method was followed to obtain the following compounds.

| Structure | Intermediates Used | Characterization |
|---|---|---|
| 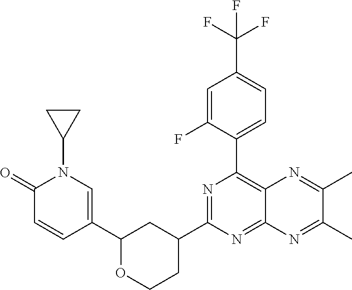 [SMILES: Cc1nc2nc(C3CCOC(c4ccc(=O)n(C5CC5)c4)C3)nc(-c3ccc(C(F)(F)F)cc3F)c2nc1C] | 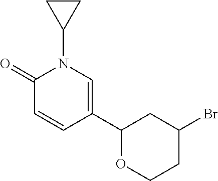 [SMILES: O=c1ccc(C2CC(Br)CCO2)cn1C1CC1] | The crude product was purified by silica gel column chromatography (0-100% PE in EtOAc and 0-50% EtOAc in MeOH) to give a crude residue which was purified by reversed-phase HPLC (0.1% FA condition) to give 1-cyclopropyl-5- (4-(4-(2-fluoro-4-(trifluoromethyl)- phenyl)-6,7-dimethylpteridin-2- yl)tetrahydro-2H-pyran-2-yl)pyridin- 2(1H)-one (Example 67, 60 mg, 0.11 mmol, 16% yield) as an oil. Then, SFC- 1-cyclopropyl-5-[4-[4-[2-fluoro-4- (trifluoromethyl)phenyl]-6,7-dimethyl- pteridin-2-yl]tetrahydropyran-2- yl]pyridin-2-one (Example was purified by SFC (Condition 16, Gradient a) to |
| | | obtain 1-cyclopropyl-5-[(2R,4R)-4-[4- |
| Example 67 | Int-A45 | [2-fluoro-4-(trifluoromethyl)phenyl]- |
| Then, SFC- | | 6,7-dimethyl-pteridin-2- |
| | | yl]tetrahydropyran-2-yl]pyridin-2-one |
| 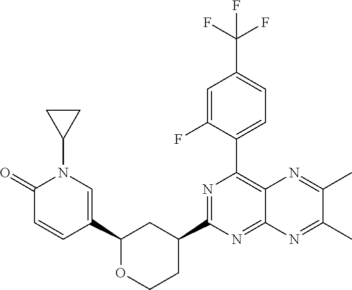 [SMILES: Cc1nc2nc([C@H]3CCO[C@@H](c4ccc(=O)n(C5CC5)c4)C3)nc(-c3ccc(C(F)(F)F)cc3F)c2nc1C] | 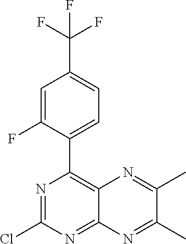 | (Example 66, 49 mg, 0.087 mmol, 8% yield), 1-cyclopropyl-5-[(2S,4R)-4-[4- [2-fluoro-4-(trifluoromethyl)phenyl]- 6,7-dimethyl-pteridin-2- yl]tetrahydropyran-2-yl]pyridin-2-one (Example 65, 95 mg, 0.176 mmol, 16% yield) as pink solid, and give 1- cyclopropyl-5-[(2R,4S)-4-[4-[2-fluoro- 4-(trifluoromethyl)phenyl]-6,7- dimethyl-pteridin-2-yl]tetrahydropyran- 2-yl]pyridin-2-one (Example 64, 51 mg, 0.0944 mmol, 8% yield) as solids. Example 66: SFC (Condition 17, Gradient a): showed that Example 66 is a ~1:1 mixture of the two trans isomers. LCMS: [M + H]⁺ = 540.4; ¹H NMR (400 MHz, CDCl₃) δ 7.85 (t, J = 7.3 Hz, 1H), 7.63 (d, J = 7.9 Hz, 1H), 7.54 (d, J = 9.1 |
| | | Hz, 1H), 7.44-7.35 (m, 2H), 6.61-6.50 |
| Example 64 | | (m, 1H), 4.37-4.24 (m, 2H), 3.86-3.75 |
| | | (m, 1H), 3.57 (ddd, J = 3.7, 11.9, 15.8 Hz, |
| 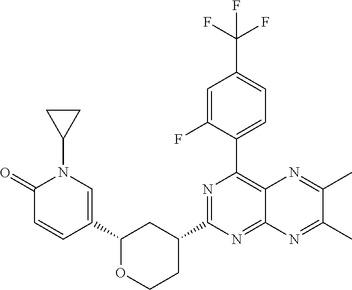 [SMILES: Cc1nc2nc([C@@H]3CCO[C@H](c4ccc(=O)n(C5CC5)c4)C3)nc(-c3ccc(C(F)(F)F)cc3F)c2nc1C] | | 1H), 3.36-3.27 (m, 1H), 2.86 (s, 3H), 2.76-2.71 (m, 3H), 2.36 (br d, J = 13.3 Hz, 1H), 2.26-2.17 (m, 2H), 2.16-2.03 (m, 1H), 1.18-1.08 (m, 2H), 0.96-0.84 (m, 2H). Example 65: LCMS: [M + H]⁺ = 540.2; purity = 97% (220 nm); Ret. time = 0.561 min. ¹H NMR (400 MHz, CDCl₃) δ 7.85 (t, J = 7.3 Hz, 1H), 7.64 (d, J = 7.6 Hz, 1H), 7.54 (br d, J = 9.5 Hz, 1H), 7.42- 7.37 (m, 2H), 6.58 (d, J = 10.1 Hz, 1H), 4.32 (brt, J = 9.3 Hz, 2H), 3.84-3.76 (m, 1H), 3.62-3.52 (m, 1H), 3.37-3.29 (m, 1H), 2.86 (s, 3H), 2.74 (s, 3H), 2.40- 2.33 (m, 1H), 2.25-2.17 (m, 2H), 2.15- 2.04 (m, 1H), 1.17-1.11 (m, 2H), 0.91- 0.88 (m, 2H). Example 64: LCMS: [M + H]⁺ = 540.2; |
| | | purity = 97% (220 nm); Ret. time = 0.558 |
| Example 65 | | min. ¹H NMR (400 MHz, CDCl₃) δ 7.88- |
| 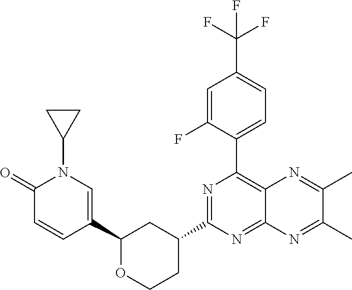 [SMILES: Cc1nc2nc([C@@H]3CCO[C@@H](c4ccc(=O)n(C5CC5)c4)C3)nc(-c3ccc(C(F)(F)F)cc3F)c2nc1C] | | 7.82 (m, 1H), 7.66-7.60 (m, 1H), 7.56- 7.51 (m, 1H), 7.38 (br s, 2H), 6.56 (br d, J = 10.0 Hz, 1H), 4.36-4.26 (m, 2H), 3.84-3.74 (m, 1H), 3.62-3.51 (m, 1H), 3.37-3.28 (m, 1H), 2.86 (s, 3H), 2.74 (s, 3H), 2.40-2.32 (m, 1H), 2.24-2.17 (m, 2H), 2.05 (s, 1H), 1.14 (br d, J = 7.3 Hz, 2H), 0.89 (br d, J = 3.0 Hz, 2H). |
| Example 66 | | |
| 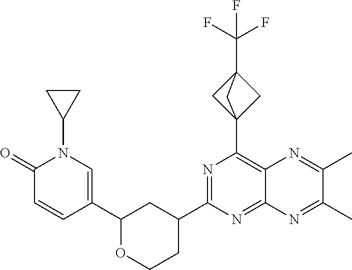 [SMILES: Cc1nc2nc(C3CCOC(c4ccc(=O)n(C5CC5)c4)C3)nc(C34CC(C(F)(F)F)(C3)C4)c2nc1C] | 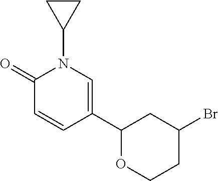 [SMILES: O=c1ccc(C2CC(Br)CCO2)cn1C1CC1] | The residue was purified by silica gel column chromatography (0-100% MeOH in EtOAc) (TLC, 9% MeOH in DCM, R_{f} = 0.6) and further purified by prep-TLC (9% MeOH in DCM, R_{f} = 0.6) to afford 150 mg to SFC separation. Example 76: LCMS: [M + H]⁺ = 512.3; purity = 84% (220 nm); Ret. time = 0.587 min. Then, SFC- Example 76 (150 mg, 84% purity) was separated by SFC (Condition 5, Gradient c) to afford 1-cyclopropyl-5-[(2R,4S)-4- [6,7-dimethyl-4-[3-(trifluoromethyl)-1- bicyclo[1.1.1]pentanyl] pteridin-2- yl]tetrahydropyran-2-yl]pyridin-2-one |
| | | (Example 74, 19 mg, 0.037 mmol, 13% |
| Example 76 | Int-A45 | yield) as a solid, and 1-cyclopropyl-5- |
| Then, SFC- | | [(2S,4R)-4-[6,7-dimethyl-4-[3- |
| | | (trifluoromethyl)-1-bicyclo[1.1.1]- |
| 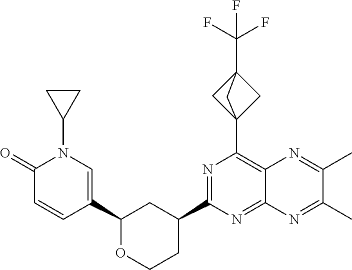 [SMILES: Cc1nc2nc([C@H]3CCO[C@@H](c4ccc(=O)n(C5CC5)c4)C3)nc(C34CC(C(F)(F)F)(C3)C4)c2nc1C] | 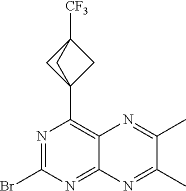 | pentanyl]-pteridin-2-yl]tetrahydropyran- 2-yl]pyridin-2-one (Example 75, 14 mg, 0.027 mmol, 9% yield) as a solid. Example 74: LCMS: [M + H]⁺ = 512.3; purity = 98% (220 nm); Ret. time = 0.580 min. ¹H NMR (400 MHz, CDCl₃) δ 7.41- 7.32 (m, 2H), 6.57-6.50 (m, 1H), 4.35- 4.22 (m, 2H), 3.86-3.64 (m, 2H), 3.49- 3.38 (m, 1H), 3.35-3.27 (m, 1H), 2.78 (d, J = 13.8 Hz, 6H), 2.64 (s, 6H), 2.28 (br d, J = 13.3 Hz, 1H), 2.16-2.08 (m, 2H), 2.06-1.95 (m, 1H), 1.19-1.06 (m, 2H), 0.88 (br d, J = 4.1 Hz, 2H). Example 75: LCMS: [M + H]⁺ = 512.3; purity = 99.3% (220 nm); Ret. time = 0.585 min. ¹H NMR (400 MHz, CDCl₃) |
| | | δ 7.40-7.34 (m, 2H), 6.56-6.50 (m, |
| Example 74 | | 1H), 4.35-4.23 (m, 2H), 3.78 (ddd, J = |
| | | 4.6, 6.7, 15.0 Hz, 1H), 3.50-3.36 (m, |
| 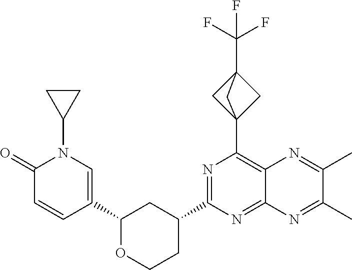 [SMILES: Cc1nc2nc([C@@H]3CCO[C@H](c4ccc(=O)n(C5CC5)c4)C3)nc(C34CC(C(F)(F)F)(C3)C4)c2nc1C] | | 1H), 3.35-3.27 (m, 1H), 2.79 (d, J = 13.9 Hz, 6H), 2.64 (s, 6H), 2.28 (br d, J = 13.4 Hz, 1H), 2.17-2.09 (m, 2H), 2.02-1.99 (m, 1H), 1.17-1.08 (m, 2H), 0.91-0.86 (m, 2H). |
| Example 75 | | |
| 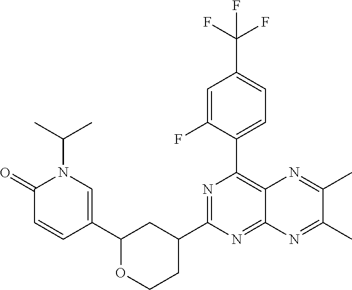 [SMILES: Cc1nc2nc(C3CCOC(c4ccc(=O)n(C(C)C)c4)C3)nc(-c3ccc(C(F)(F)F)cc3F)c2nc1C] | 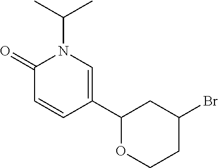 | The residue was purified by silica gel column chromatography (0-100% MeOH in EtOAc) (TLC, 6% MeOH in DCM, R_{f} = 0.60) to afford a solid. The product was purified by Prep-TLC (6% MeOH in DCM R_{f} = 0.55) twice to remove color and afford the desired product 5-[4-[4-[2-fluoro-4-(trifluoro- methyl)phenyl]-6,7-dimethyl-pteridin-2- yl]tetrahydropyran-2-y1]-1-isopropyl- pyridin-2-one (Example 70, 70 mg, 0.106 mmol, 12% yield) as a solid. LCMS: [M + H]⁺ = 542.1; purity = 84% (220 nm); Ret. time = 0.576 min. Then, SFC- The residue (Example 70) was separated by SFC (Condition 1, Gradient a) to afford 1-isopropyl-5-[(2R,4S)-4-[4-[2- |
| | Int-A47 | fluoro-4-(trifluoromethyl)-phenyl]-6,7- |
| Example 70 | | dimethyl-pteridin-2-yl]tetrahydropyran- |
| Then, SFC- | | 2-yl]pyridin-2-one (Example 68, 12 mg, |
| | | 0.0218 mmol, 26% yield) as a solid. |
| 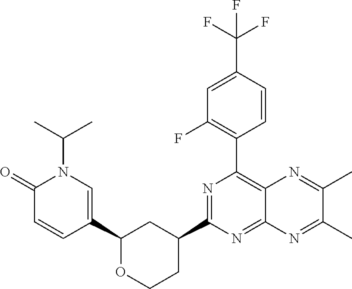 [SMILES: Cc1nc2nc([C@H]3CCO[C@@H](c4ccc(=O)n(C(C)C)c4)C3)nc(-c3ccc(C(F)(F)F)cc3F)c2nc1C] | 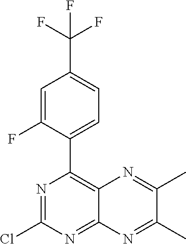 | Example 68: LCMS: [M + H]⁺ = 542.2; purity = 95% (220 nm); Ret. time = 0.580 min. ¹H NMR (400 MHz, CDCl₃) δ 7.86 (t, J = 7.2 Hz, 1H), 7.63 (br d, J = 7.9 Hz, 1H), 7.53 (d, J = 9.4 Hz, 1H), 7.40 (br d, J = 3.6 Hz, 2H), 6.64-6.45 (m, 1H), 5.49- 5.13 (m, 1H), 4.53-4.19 (m, 2H), 3.92- 3.72 (m, 1H), 3.65-3.51 (m, 1H), 2.86 (s, 3H), 2.74 (s, 3H), 2.46-2.30 (m, 1H), 2.27-2.16 (m, 2H), 2.14-1.99 (m, 1H), 1.37 (br d, J = 5.3 Hz, 6H). The other part (20 mg with 88% purity) was separated again by SFC (Condition 1, Gradient a) to afford 1-isopropyl-5- [(2S,4R)-4-[4-[2-fluoro-4- (trifluoromethyl)-phenyl]-6,7-dimethyl- pteridin-2-yl]tetrahydropyran-2- yl]pyridin-2-one (Example 69, 6.7 mg, |
| | | 0.012 mmol, 15% yield) as a solid. |
| Example 68 | | Example 69: LCMS: [M + H]⁺ = 542.2; |
| | | purity = 97% (220 nm); Ret. time = 0.579 |
| 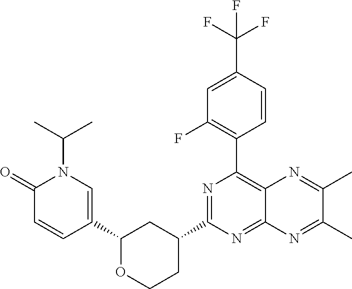 [SMILES: Cc1nc2nc([C@@H]3CCO[C@H](c4ccc(=O)n(C(C)C)c4)C3)nc(-c3ccc(C(F)(F)F)cc3F)c2nc1C] | | min. ¹H NMR (400 MHz, CDCl₃) δ 7.86 (t, J = 7.3 Hz, 1H), 7.63 (d, J = 7.9 Hz, 1H), 7.54 (d, J = 9.4 Hz, 1H), 7.45-7.30 (m, 2H), 6.66-6.50 (m, 1H), 5.40-5.21 (m, 1H), 4.53-4.21 (m, 2H), 3.85-3.71 (m, 1H), 3.63-3.52 (m, 1H), 2.86 (s, 3H), 2.74 (s, 3H), 2.37 (br d, J = 13.3 Hz, 1H), 2.28-2.19 (m, 2H), 2.16-2.02 (m, 1H), 1.37 (br d, J = 5.4 Hz, 6H). |
| Example 69 | | |
| 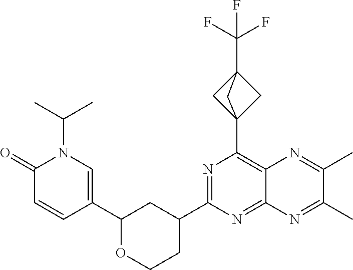 [SMILES: Cc1nc2nc(C3CCOC(c4ccc(=O)n(C(C)C)c4)C3)nc(C34CC(C(F)(F)F)(C3)C4)c2nc1C] | 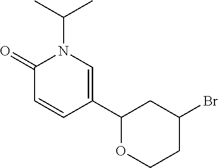 [SMILES: CC(C)n1cc(C2CC(Br)CCO2)ccc1=O] | The combined organic layers were dried over anhydrous Na₂SO₄, filtered, and concentrated under reduced pressure to give the crude residue (Example 79). Then, SFC- The crude (Example 79) was purified by silica gel column chromatography (9% MeOH in EtOAc, R_{f} = 0.3) to give 80 mg product as a solid. Then, 80 mg product was separated by SFC (Condition 6, Gradient c) and lyophilized to give Crude A and 1-isopropyl-5-[(2R,4S)-4- [6,7-dimethyl-4-[3-(trifluoromethyl)-1- bicyclo[1.1.1]-pentanyl]pteridin-2- yl]tetrahydropyran-2-yl]pyridin-2-one (Example 77, 18 mg, 0.032 mmol, 14% |
| | Int-A47 | yield) as a solid. Example 77: LCMS: |
| Example 79 | | [M + H]⁺ = 514.2; purity = 95% (UV 220 |
| Then, SFC- | | nm); Ret. time = 0.601 min. ¹H NMR |
| | | (400 MHz, CDCl₃) δ 7.40-7.32 (m, 2H), |
| 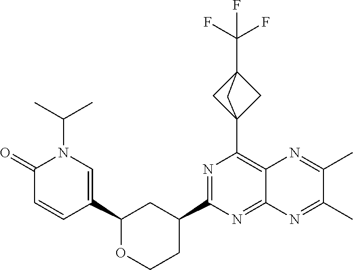 [SMILES: Cc1nc2nc([C@H]3CCO[C@@H](c4ccc(=O)n(C(C)C)c4)C3)nc(C34CC(C(F)(F)F)(C3)C4)c2nc1C] | 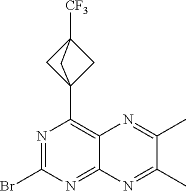 [SMILES: Cc1nc2nc(Br)nc(C34CC(C(F)(F)F)(C3)C4)c2nc1C] | 6.56 (d, J = 9.3 Hz, 1H), 5.38-5.19 (m, 1H), 4.39-4.25 (m, 2H), 3.84-3.73 (m, 1H), 3.55-3.37 (m, 1H), 2.81 (s, 3H), 2.77 (s, 3H), 2.68-2.62 (m, 6H), 2.34- 2.24 (m, 1H), 2.18-2.08 (m, 2H), 2.06- 1.96 (m, 1H), 1.38-1.34 (m, 6H). SFC: 100% ee. Crude A (40 mg) was further separated by SFC (Condition 6, Gradient d) and lyophilized to give Crude B and Crude C. Crude C (18 mg) was purified by prep-TLC (9% MeOH in DCM, R_{f} = 0.3) (×3) and concentrated under reduced pressure to give 1-isopropyl-5- [(2S,4R)-4-[6,7-dimethyl-4-[3- (trifluoromethyl)-1-bicyclo[1.1.1]- |
| | | pentanyl]pteridin-2-yl]tetrahydropyran- |
| Example 77 | | 2-yl]pyridin-2-one (Example 78, 3.6 mg, |
| | | 0.006 mmol, 3% yield) as a solid. |
| 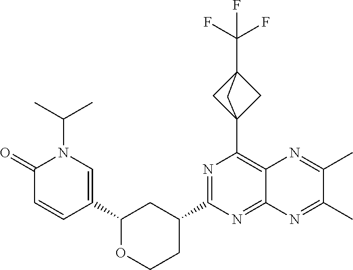 [SMILES: Cc1nc2nc([C@@H]3CCO[C@H](c4ccc(=O)n(C(C)C)c4)C3)nc(C34CC(C(F)(F)F)(C3)C4)c2nc1C] | | Example 78: LCMS: [M + H]⁺ = 514.3; purity = 92% (UV 220 nm); Ret. time = 0.608 min. ¹H NMR (400 MHz, CDCl₃) δ 7.40-7.33 (m, 2H), 6.56 (d, J = 9.1 Hz, 1H), 5.33-5.21 (m, 1H), 4.33 (br dd, J = 2.1, 13.3 Hz, 2H), 3.86-3.72 (m, 1H), 3.53-3.39 (m, 1H), 2.80 (s, 3H), 2.78- 2.74 (m, 3H), 2.64 (s, 6H), 2.33-2.26 (m, 1H), 2.22-2.10 (m, 2H), 2.02-2.00 (m, 1H), 1.36 (dd, J = 1.2, 6.8 Hz, 6H). SFC: 100% ee. |
| Example 78 | | |
| 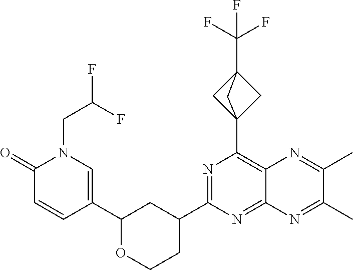 [SMILES: Cc1nc2nc(C3CCOC(c4ccc(=O)n(CC(F)F)c4)C3)nc(C34CC(C(F)(F)F)(C3)C4)c2nc1C] | 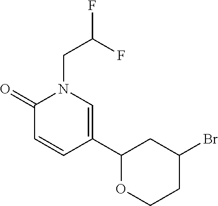 [SMILES: O=c1ccc(C2CC(Br)CCO2)cn1CC(F)F] | The crude 1-(2,2-difluoroethyl)-5-[4- [6,7-dimethyl-4-[3-(trifluoro-methyl)-1- bicyclo[1.1.1]pentanyl]pteridin-2- yl]tetrahydropyran-2-yl]pyridin-2-one (Example 73, 144 mg, 0.268 mmol, 100% yield) was obtained as a solid. Then, SFC- 1-(2,2-difluoroethyl)-5-[4-[6,7- dimethyl-4-[3-(trifluoromethyl)-1- bicyclo[1.1.1]pentanyl]pteridin-2- yl]tetrahydropyran-2-yl]pyridin-2-one (Example 73, 1 equiv., 400 mg, 0.747 mmol) was purified by Prep-TLC (100% EtOAc, desired product Rₜ = 0.4) to give crude product (130 mg). The crude product was purified by SFC (Condition 11, Gradient c) and lyophilized to give |
| | Int-A48 | the 1-(2,2-difluoroethyl)-5-[(2R,4S)-4- |
| Example 73 | | [6, 7-dimethyl-4-[3-(trifluoromethyl)-1- |
| Then, SFC- | | bicyclo[1.1.1]pentanyl]pteridin-2- |
| | | yl]tetrahydropyran-2-yl]pyridin-2-one |
| 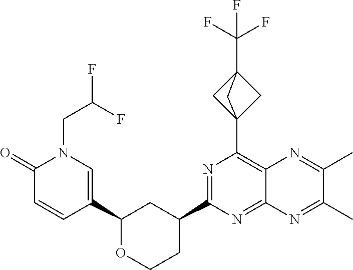 [SMILES: Cc1nc2nc([C@H]3CCO[C@@H](c4ccc(=O)n(CC(F)F)c4)C3)nc(C34CC(C(F)(F)F)(C3)C4)c2nc1C] | 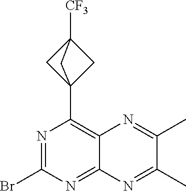 | (Example 71, 27 mg, 0.045 mmol, 6% yield) as a solid and 1-(2,2- difluoroethyl)-5-[(2S,4R)-4-[6,7- dimethyl-4-[3-(trifluoro-methyl)-1- bicyclo-[1.1.1]pentanyl]-pteridin-2- yl]tetrahydropyran-2-yl]pyridin-2-one (Example 72, 27 mg, 0.047 mmol, 6% yield) as a solid. Example 71: LCMS: [M + H]⁺ = 536.0; purity = 93% (220 nm); Ret. time = 0.608 min. HPLC: Ret. time = 2.020 min, 89% purity at 220 nm. ¹H NMR (400 MHz, CDCl₃) δ 7.46 (dd, J = 2.4, 9.4 Hz, 1H), 7.33 (d, J = 1.8 Hz, 1H), 6.58 (d, J = 9.5 Hz, 1H), 6.31-5.94 (m, 1H), 4.36-4.18 (m, 4H), 3.78 (dt, J = 3.3, 11.5 Hz, 1H), 3.51-3.40 (m, 1H), 2.79 (d, J = 14.1 Hz, |
| | | 6H), 2.64 (s, 6H), 2.32 (br d, J = 13.6 Hz, |
| Example 71 | | 1H), 2.20-2.08 (m, 2H), 2.07-1.94 (m, |
| | | 1H). |
| 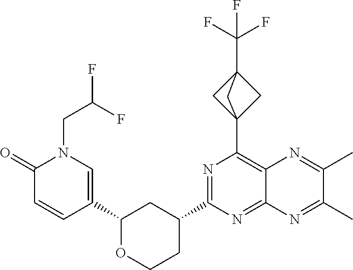 [SMILES: Cc1nc2nc([C@@H]3CCO[C@H](c4ccc(=O)n(CC(F)F)c4)C3)nc(C34CC(C(F)(F)F)(C3)C4)c2nc1C] | | Example 72: LCMS: [M + H]⁺ = 536.0; purity = 94% (220 nm); Ret. time = 0.606 min. HPLC: Ret. time = 2.019 min, 92% purity at 220 nm. ¹H NMR (400 MHz, CDCl₃) δ 7.46 (dd, J = 2.4, 9.4 Hz, 1H), 7.33 (d, J = 2.0 Hz, 1H), 6.58 (d, J = 9.5 Hz, 1H), 6.29-5.96 (m, 1H), 4.36-4.17 (m, 4H), 3.78 (dt, J = 3.3, 11.6 Hz, 1H), 3.51-3.39 (m, 1H), 2.79 (d, J = 14.3 Hz, 6H), 2.64 (s, 6H), 2.32 (br dd, J = 1.5, 13.1 Hz, 1H), 2.21-2.09 (m, 2H), 2.07- 1.93 (m, 1H). |
| Example 72 | | |
| 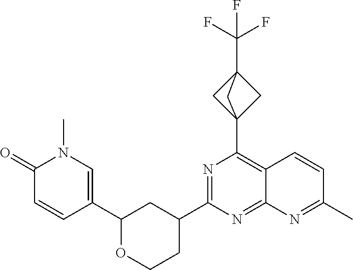 [SMILES: Cc1ccc2c(C34CC(C(F)(F)F)(C3)C4)nc(C3CCOC(c4ccc(=O)n(C)c4)C3)nc2n1] | 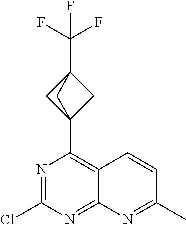 [SMILES: Cc1ccc2c(C34CC(C(F)(F)F)(C3)C4)nc(Cl)nc2n1] | The crude residue (150 mg) was purified by silica gel column chromatography (0- 100% EtOAc in PE & EtOAc/MeOH) (TLC, 25% MeOH in EtOAc, R_{f} = 0.4) to give 71 mg crude product (Example 319) as a solid. The crude product was purified again by SFC (Condition 11, Gradient a) to give Crude A (20 mg, 78% purity) and Crude B (20 mg, 81% purity). Crude A (20 mg) was purified by SFC (Condition 11, Gradient b), then by prep- HPLC (Condition 7, Gradient a) to give 1-methyl-5-[(2R,4S)-4-[7-methyl-4-[3- (trifluoromethyl)-1-bicyclo[1.1.1]- pentanyl]pyrido[2,3-d]pyrimidin-2- yl]tetrahydropyran-2-yl]pyridin-2-one |
| | Int-B18 | (Example 320, 4.2 mg, 0.01 mmol, 3% |
| Example 319 | | yield) as a solid. LCMS: [M + H]⁺ = |
| Then, SFC- | | 471.0; Ret. time = 0.516 min. HPLC: |
| | | Ret. time = 1.495 min. ¹H NMR (400 |
| 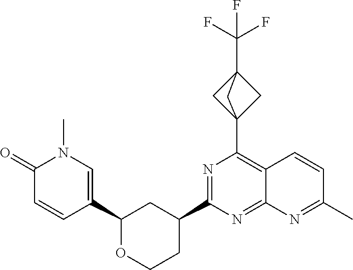 [SMILES: Cc1ccc2c(C34CC(C(F)(F)F)(C3)C4)nc([C@H]3CCO[C@@H](c4ccc(=O)n(C)c4)C3)nc2n1] | | MHz, CDCl₃) δ 8.44 (d, J = 8.5 Hz, 1H), 7.46-7.35 (m, 3H), 6.57 (d, J = 10.3 Hz, 1H), 4.38-4.24 (m, 2H), 3.79 (dt, J = 3.7, 11.2 Hz, 1H), 3.55 (s, 3H), 3.48-3.36 (m, 1H), 2.83 (s, 3H), 2.63 (s, 6H), 2.31 (br d, J = 13.3 Hz, 1H), 2.18-2.08 (m, 2H), 2.03 (br d, J = 13.0 Hz, 1H). Crude B (20 mg) was purified by HPLC (Condition 7, Gradient a) to give 1- methyl-5-[(2S,4R)-4-[7-methyl-4-[3- (trifluoromethyl)-1-bicyclo[1.1.1]- pentanyl]pyrido[2,3-d]pyrimidin-2- yl]tetrahydropyran-2-yl]pyridin-2-one (Example 321, 5.8 mg, 0.012 mmol, 4% yield) as a solid. LCMS: [M + H]⁺ = 471.2; Ret. time = 0.509 min. HPLC: Ret. time = 1.491 min. ¹H NMR (400 |
| | | MHz, CDCl₃) δ 8.44 (d, J = 8.5 Hz, 1H), |
| Example 320 | | 7.47-7.34 (m, 3H), 6.57 (d, J = 10.1 Hz, |
| | | 1H), 4.37-4.21 (m, 2H), 3.86-3.72 (m, |
| 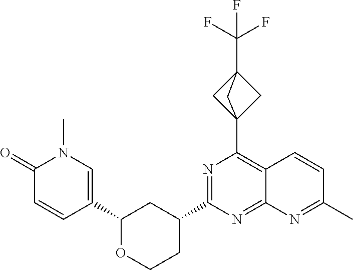 [SMILES: Cc1ccc2c(C34CC(C(F)(F)F)(C3)C4)nc([C@@H]3CCO[C@H](c4ccc(=O)n(C)c4)C3)nc2n1] | | 1H), 3.55 (s, 3H), 3.48-3.37 (m, 1H), 2.83 (s, 3H), 2.63 (s, 6H), 2.31 (br d, J = 13.1 Hz, 1H), 2.18-1.98 (m, 3H). |
| Example 321 | | |
| 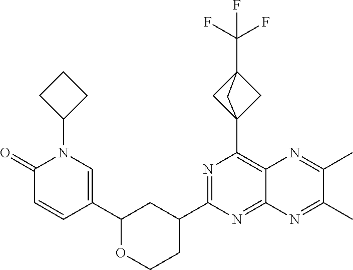 [SMILES: Cc1nc2nc(C3CCOC(c4ccc(=O)n(C5CCC5)c4)C3)nc(C34CC(C(F)(F)F)(C3)C4)c2nc1C] | 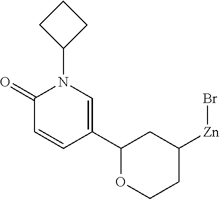 | The procedure yielded 1-cyclobutyl-5- (4-(6,7-dimethyl-4-(3- (trifluoromethyl)bicyclo[1.1.1]pentan-1- yl)pteridin-2-yl)tetrahydro-2H-pyran-2- yl)pyridin-2(1H)-one (Example 344). This was further further purified by SFC (Condition 20, Gradient a) to give an enriched mixture A as a solid. Mixture A was further purified into two absolute configuration products by SFC (Condition 21, Gradient a) to obtain 1- cyclobutyl-5-[(2R,4S)-4-[6,7-dimethyl- 4-[3-(trifluoromethyl)-1 bicyclo[1.1.1]pentanyl]pteridin-2- yl]tetrahydropyran-2-yl]pyridin-2-one (Example 94, 28 mg, 0.0525 mmol, 6% yield) and 1-cyclobutyl-5-[(2S,4R)-4- |
| | Int-F15 | [6,7-dimethyl-4-[3-(trifluoromethyl)-1- |
| Example 344 | | bicyclo[1.1.1 ]pentanyl]pteridin-2- |
| Then, SFC- | | yl]tetrahydropyran-2-yl]pyridin-2-one |
| | | (Example 95, 27 mg, 0.0495 mmol, 5% |
| 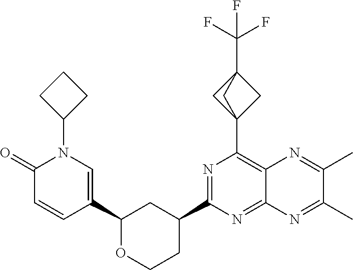 [SMILES: Cc1nc2nc([C@H]3CCO[C@@H](c4ccc(=O)n(C5CCC5)c4)C3)nc(C34CC(C(F)(F)F)(C3)C4)c2nc1C] | 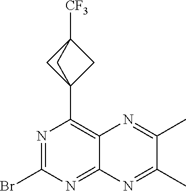 | yield) as solids. Example 94: LCMS: [M + H]⁺ = 526.3. ¹H NMR (400 MHz, CDCl₃) δ 1.82- 1.90 (m, 2 H) 1.99-2.08 (m, 1 H) 2.12- 2.19 (m, 2 H) 2.20-2.34 (m, 3 H) 2.46- 2.54 (m, 2 H) 2.65 (s, 6 H) 2.78 (s, 3 H) 2.82 (s, 3 H) 3.42-3.52 (m, 1 H) 3.76- 3.85 (m, 1 H) 4.28-4.38 (m, 2 H) 5.10- 5.20 (m, 1 H) 6.53 (d, J = 9.4 Hz, 1 H) 7.37 (dd, J=9.4, 2.50 Hz, 1 H) 7.51 (d, J = 2.4 Hz, 1 H). Example 95: LCMS: [M + H]⁺ = 526.4. ¹H NMR (400 MHz, CDCl₃) δ 1.74- 1.82 (m, 2 H) 1.91-2.00 (m, 1 H) 2.05- 2.11 (m, 2 H) 2.13-2.25 (m, 3 H) 2.38- 2.46 (m, 2 H) 2.58 (s, 6 H) 2.72 (d, J = 14.0 Hz, 6 H) 3.32-3.45 (m, 1 H) 3.69- |
| | | 3.78 (m, 1 H) 4.19-4.32 (m, 2 H) 5.01- |
| Example 94 | | 5.12 (m, 1 H) 6.45 (d, J = 9.3 Hz, 1 H) |
| | | 7.29 (dd, J = 9.4, 2.5 Hz, 1 H) 7.43 (d, |
| 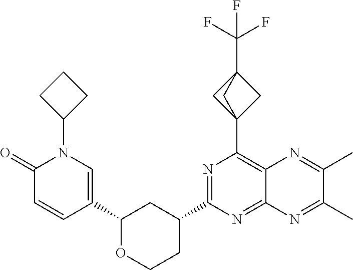 [SMILES: Cc1nc2nc([C@@H]3CCO[C@H](c4ccc(=O)n(C5CCC5)c4)C3)nc(C34CC(C(F)(F)F)(C3)C4)c2nc1C] | | J = 2.4 Hz, 1 H). |
| Example 95 | | |

### Method 4: Synthesis of Examples 56, 57, 59, 161-163

### Example 59:5-[(2R,6R)-4-[6,7-dimethyl-4-[3-(trifluoromethyl)-1-bicyclo[1.1.1]pentanyl]pteridin-2-yl]-6-methyl-tetrahydropyran-2-yl]pyridin-2-ol

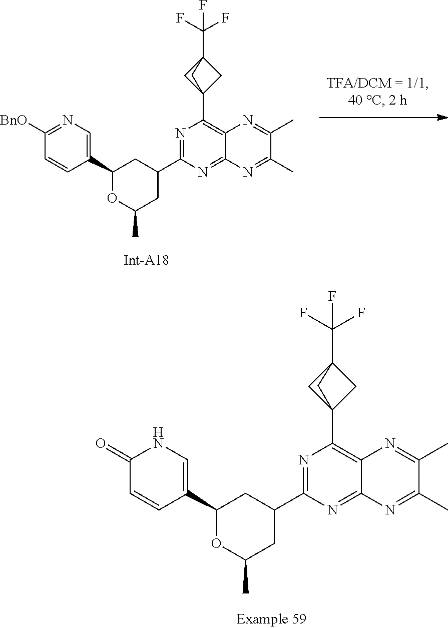 [SMILES: Cc1nc2nc(C3C[C@@H](C)O[C@@H](c4ccc(OCc5ccccc5)nc4)C3)nc(C34CC(C(F)(F)F)(C3)C4)c2nc1C ; Cc1nc2nc(C3C[C@@H](C)O[C@@H](c4ccc(=O)[nH]c4)C3)nc(C34CC(C(F)(F)F)(C3)C4)c2nc1C]

To a solution of 2-[(2R,6R)-2-(6-benzyloxy-3-pyridyl)-6-methyl-tetrahydropyran-4-yl]-6,7-dimethyl-4-[3-(trifluoromethyl)-1-bicyclo[1.1.1]pentanyl]pteridine (Int-A18, 1 equiv., 700 mg, 1.22 mmol) in DCM (10 mL) was added TFA (1 equiv., 10 mL, 1.22 mmol), and the mixture was stirred for 2 h at 40° C. LCMS showed desired product (95%). The mixture was quenched by 100 mL saturation NaHCO₃ solution, extracted with EtOAc (50 mL×3), the combined organic layers were dried over anhydrous Na₂SO₄, filtered, and concentrated under reduced pressure to give a crude residue. The residue was purified by silica gel column chromatography (0-100% EtOAc in PE, then 0-50% MeOH in EtOAc) (TLC, 9% MeOH in DCM, desired product R_{f}=0.4) to give the 5-[(2R,6R)-4-[6,7-dimethyl-4-[3-(trifluoromethyl)-1-bicyclo[1.1.1]pentanyl]pteridin-2-yl]-6-methyl-tetrahydropyran-2-yl]pyridin-2-ol (Example 59, 500 mg, 1.03 mmol, 85% yield) as a solid. LCMS: [M+H]⁺=486.1, purity=96% (220 nm), Ret. time=0.572 min. ¹H NMR (400 MHz, CDCl₃) δ 12.21-11.51 (m, 1H), 7.60 (dd, J=2.3, 9.4 Hz, 1H), 7.43 (d, J=1.8 Hz, 1H), 6.61 (br d, J=9.4 Hz, 1H), 4.41 (br d, J=10.5 Hz, 1H), 3.90-3.77 (m, 1H), 3.53-3.41 (m, 1H), 2.79 (d, J=13.1 Hz, 5H), 2.65 (s, 6H), 2.31-2.11 (m, 2H), 1.32 (d, J=6.1 Hz, 3H).

### Example 57:2-[(2R,6R)-2-(6-methoxy-3-pyridyl)-6-methyl-tetrahydropyran-4-yl]-6,7-dimethyl-4-[3-(trifluoromethyl)-1-bicyclo[1.1.1]pentanyl]pteridine

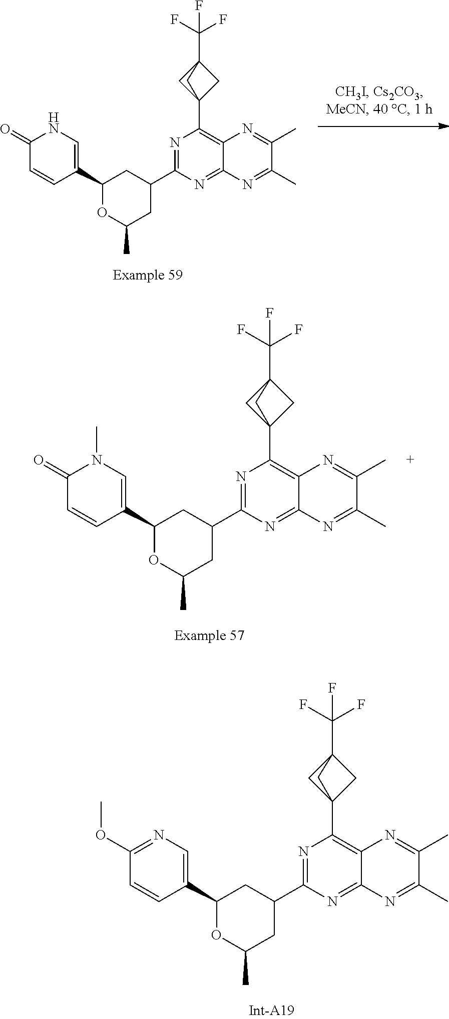 [SMILES: Cc1nc2nc(C3C[C@@H](C)O[C@@H](c4ccc(=O)[nH]c4)C3)nc(C34CC(C(F)(F)F)(C3)C4)c2nc1C ; Cc1nc2nc(C3C[C@@H](C)O[C@@H](c4ccc(=O)n(C)c4)C3)nc(C34CC(C(F)(F)F)(C3)C4)c2nc1C ; COc1ccc([C@H]2CC(c3nc(C45CC(C(F)(F)F)(C4)C5)c4nc(C)c(C)nc4n3)C[C@@H](C)O2)cn1]

To a solution of 5-[rac-(2R,6R)-4-[6,7-dimethyl-4-[3-(trifluoromethyl)-1-bicyclo[1.1.1]pentanyl]pteridin-2-yl]-6-methyl-tetrahydropyran-2-yl]-1H-pyridin-2-one (Example 59, 1 equiv., 220 mg, 0.453 mmol) and Cs₂CO₃ (4 equiv., 591 mg, 1.81 mmol) in MeCN (3 mL) was added CH₃I (4 equiv., 257 mg, 1.81 mmol). The solution was stirred 30° C. for 2 hours. LCMS showed two peaks with desired product detected (3%; 65%). The mixture was quenched by 50 mL H₂O, extracted with EtOAc (50 mL×3), the combined organic layers were dried over anhydrous Na₂SO₄, filtered, and concentrated under reduced pressure to give the crude product. The crude product was purified by silica gel column chromatography (0-100% EtOAc in PE, then 0-50% MeOH in EtOAc) to give 2-[(2R,6R)-2-(6-methoxy-3-pyridyl)-6-methyl-tetrahydropyran-4-yl]-6,7-dimethyl-4-[3-(trifluoromethyl)-1-bicyclo[1.1.1]pentanyl]pteridine (Example 57, 4.9 mg, 0.009 mmol, 2% yield) as a solid (TLC, 67% EtOAc in PE, R_{f}=0.3), and byproduct 5-[(2R,6R)-4-[6,7-dimethyl-4-[3-(trifluoromethyl)-1-bicyclo[1.1.1]pentanyl]pteridin-2-yl]-6-methyl-tetrahydropyran-2-yl]-1-methyl-pyridin-2-one (200 mg, 0.400 mmol, 88% yield) as a solid (TLC, 67% EtOAc in PE, R_{f}=0.1). Example 57: LCMS: [M+H]⁺=500.2, purity=94% (220 nm), Ret. time=0.654 min. ¹H NMR (400 MHz, CDCl₃) δ 8.19 (d, J=2.3 Hz, 1H), 7.70 (dd, J=2.4, 8.6 Hz, 1H), 6.78-6.69 (m, 1H), 4.59 (dd, J=1.8, 11.4 Hz, 1H), 3.88 (ddd, J=1.8, 6.1, 11.1 Hz, 1H), 3.52 (tt, J=3.8, 12.1 Hz, 1H), 2.79 (d, J=14.3 Hz, 6H), 2.65 (s, 6H), 2.28 (td, J=1.7, 13.1 Hz, 1H), 2.23-2.16 (m, 1H), 2.05-1.97 (m, 1H), 1.90-1.79 (m, 1H), 1.35 (d, J=6.1 Hz, 3H). SFC showed ee ~67%.

### Example 56:5-[(2R,4S,6R)-4-[6,7-dimethyl-4-[3-(trifluoromethyl)-1-bicyclo[1.1.1]-pentanyl]pteridin-2-yl]-6-methyl-tetrahydropyran-2-yl]-1-methyl-pyridin-2-one

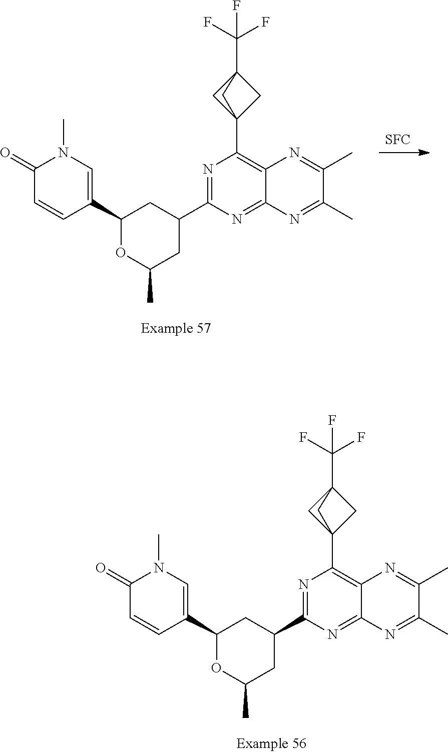

The residue (Example 57) was purified by SFC (Condition 4, Gradient b) and lyophilized to give 5-[(2R,4S,6R)-4-[6,7-dimethyl-4-[3-(trifluoromethyl)-1-bicyclo[1.1.1]-pentanyl]pteridin-2-yl]-6-methyl-tetrahydropyran-2-yl]-1-methyl-pyridin-2-one (Example 56, 118 mg, 0.232 mmol, 58% yield) as a solid. LCMS: [M+H]⁺=500.2, purity=99% (220 nm), Ret. time=0.591 min. ¹H NMR (400 MHz, CDCl₃) δ 7.46-7.37 (m, 2H), 6.57 (d, J=9.5 Hz, 1H), 4.39 (dd, J=1.6, 11.4 Hz, 1H), 3.93-3.78 (m, 1H), 3.58-3.53 (m, 3H), 3.47 (tt, J=3.7, 12.1 Hz, 1H), 2.80 (d, J=14.5 Hz, 6H), 2.65 (s, 6H), 2.32-2.25 (m, 1H), 2.20 (td, J=1.7, 13.3 Hz, 1H), 1.94 (q, J=12.1 Hz, 1H), 1.83-1.71 (m, 1H), 1.34 (d, J=6.3 Hz, 3H). SFC showed ee ~100%.

An analogous method was followed to obtain the following compounds.

| Structure | Starting Material | Characterization |
|---|---|---|
| 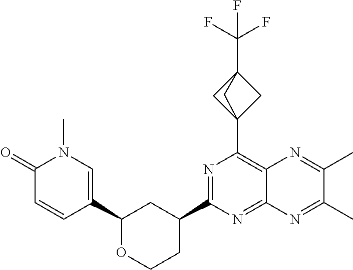 [SMILES: Cc1nc2nc([C@H]3CCO[C@@H](c4ccc(=O)n(C)c4)C3)nc(C34CC(C(F)(F)F)(C3)C4)c2nc1C] | 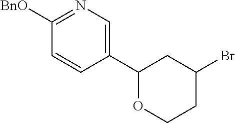 [SMILES: BrC1CCOC(c2ccc(OCc3ccccc3)nc2)C1] | The residue was purified by reversed-phase HPLC (60-65% [water (0.1% NH₃•H₂O)-ACN], Flow rate: 60 mL/min) to afford racemate (85 mg) as a gum. Then the racemate (85 mg) was purified by SFC (Condition 6, Gradient e) to give three impure enantiomers (Crude A (15 mg), Crude B (22 mg), and Crude C (19 mg)). Crude A (15 mg) was further purified by prep-HPLC (Condition 5, Gradient d) to afford 1-methyl-5- [(2S,4S)-4-[6, 7-dimethyl-4-[3- (trifluoromethyl)-1-bicyclo[1.1.1] pentanyl]pteridin-2- |
| | | yl]tetrahydropyran-2-yl]pyrimidin- |
| Example 161 | Int-B16 | 2-one (Example 163, 3.1 mg, 0.006 |
| | | mmol, 1% yield) as a solid. LCMS: |
| 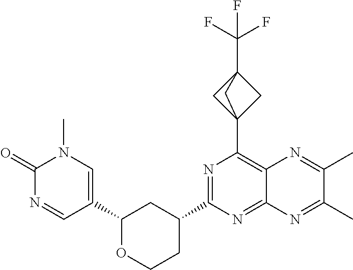 [SMILES: Cc1nc2nc([C@@H]3CCO[C@H](c4cnc(=O)n(C)c4)C3)nc(C34CC(C(F)(F)F)(C3)C4)c2nc1C] | | [M + H]⁺ = 487.1; purity = 94% (220 nm); Ret. time = 0.537 min; ¹H NMR (400 MHz, CDCl₃, 301 K) δ 8.62 (d, J = 3.1 Hz, 1H), 7.72 (d, J = 3.1 Hz, 1H), 4.67 (dd, J = 2.0, 10.5 Hz, 1H), 4.02-3.93 (m, 1H), 3.83 (dt, J = 2.3, 11.8 Hz, 1H), 3.73-3.65 (m, 1H), 3.59 (s, 3H), 2.84-2.77 (m, 7H), 2.66 (s, 6H), 2.58 (br d, J = 13.9 Hz, 1H), 2.27-2.15 (m, 1H), 2.07 (ddd, J = 5.3, 10.7, 13.6 Hz, 1H); ee = 81%. Crude B (22 mg) was further purified by prep-HPLC (Condition 5, Gradient e) to afford 1-methyl-5- [(2S,4R)-4-[6,7-dimethyl-4-[3- |
| | | (trifluoromethyl)-1-bicyclo[1.1.1]- |
| Example 162 | | pentanyl]pteridin-2- |
| | | yl]tetrahydropyran-2-yl]pyrimidin- |
| 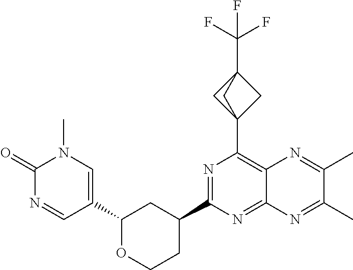 [SMILES: Cc1nc2nc([C@H]3CCO[C@H](c4cnc(=O)n(C)c4)C3)nc(C34CC(C(F)(F)F)(C3)C4)c2nc1C] | | 2-one (Example 162, 2.8 mg, 0.0056 mmol, 1% yield) as a solid. LCMS: [M + H]⁺ = 487.1; purity = 97% (220 nm); Ret. time = 0.530 min; ¹H NMR (400 MHz, CDCl₃, 301 K) δ 8.63 (d, J = 3.3 Hz, 1H), 7.77 (d, J = 3.0 Hz, 1H), 4.43 (dd, J = 1.6, 11.5 Hz, 1H), 4.29 (br dd, J = 3.4, 11.3 Hz, 1H), 3.81 (dt, J = 2.4, 12.0 Hz, 1H), 3.59 (s, 3H), 3.47 (tt, J = 3.7, 11.9 Hz, 1H), 2.80 (d, J = 14.0 Hz, 6H), 2.65 (s, 6H), 2.37 (br d, J = 13.4 Hz, 1H), 2.22-2.15 (m, 1H), 1.98 (s, 2H); ee = 84%. Crude C (19 mg) was further purified by prep-HPLC (Condition |
| | | 5, Gradient f) to afford 1-methyl-5- |
| Example 163 | | [(2R,4S)-4-[6,7-dimethyl-4-[3- |
| | | (trifluoromethyl)-1-bicyclo[1.1.1]- |
| | | pentanyl]pteridin-2- |
| | | yl\|tetrahydropyran-2-yl]pyrimidin- |
| | | 2-one (Example 161, 3.9 mg, 0.008 |
| | | mmol, 2% yield) as a solid. LCMS: |
| | | [M + H]⁺ = 487.2; purity = 98% (220 |
| | | nm); Ret. time = 0.530 min; ¹H |
| | | NMR (400 MHz, CDCl₃, 298 K) δ |
| | | (ppm) = 8.63 (br d, J = 2.1 Hz, 1H), |
| | | 7.78 (br d, J = 2.6 Hz, 1H), 4.43 (br |
| | | d, J = 11.3 Hz, 1H), 4.29 (br dd, J = |
| | | 3.8, 11.4 Hz, 1H), 3.86-3.75 (m, |
| | | 1H), 3.59 (s, 3H), 3.53-3.42 (m, |
| | | 1H), 2.82 (s, 3H), 2.78 (s, 3H), 2.65 |
| | | (s, 6H), 2.37 (br d, J = 13.3 Hz, 1H), |
| | | 2.22-2.14 (m, 1H), 2.12-1.98 (m, |
| | | 2H); ee = 88%. |

### Method 5: Synthesis of Examples 102, 134, 142

### Example 102:1-cyclopropyl-5-[(2R,4S,6R)-4-[6,7-dimethyl-4-[3-(trifluoromethyl)-1-bicyclo[1.1.1]pentanyl]pteridin-2-yl]-6-methyl-tetrahydropyran-2-yl]pyridin-2-one

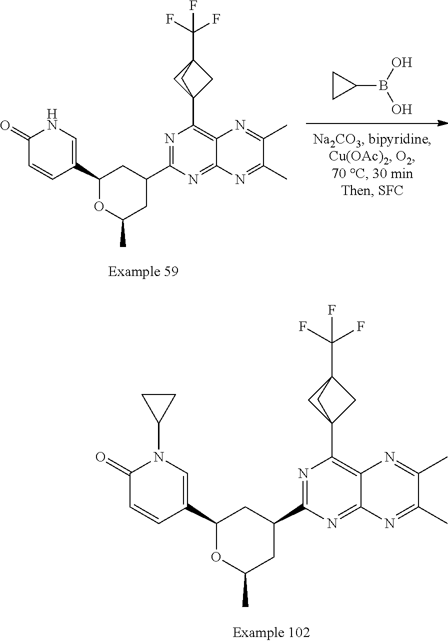 [SMILES: Cc1nc2nc(C3C[C@@H](C)O[C@@H](c4ccc(=O)[nH]c4)C3)nc(C34CC(C(F)(F)F)(C3)C4)c2nc1C ; OB(O)C1CC1 ; Cc1nc2nc([C@H]3C[C@@H](C)O[C@@H](c4ccc(=O)n(C5CC5)c4)C3)nc(C34CC(C(F)(F)F)(C3)C4)c2nc1C]

To a mixture of 5-[(2R,6R)-4-[6,7-dimethyl-4-[3-(trifluoromethyl)-1-bicyclo[1.1.1]pentanyl]pteridin-2-yl]-6-methyl-tetrahydropyran-2-yl]-1H-pyridin-2-one (Example 59, 1 equiv., 230 mg, 0.474 mmol), cyclopropylboronic acid (2 equiv., 81 mg, 0.947 mmol) in DCE (12 mL) was added Na₂CO₃ (3 equiv., 151 mg, 1.42 mmol), Cu(OAc)₂ (1.5 equiv., 129 mg, 0.711 mmol) and bipyridine (1 equiv., 74 mg, 0.474 mmol), then the mixture was stirred for 0.5 h at 70° C. under 02 (15 psi). LCMS showed starting material was consumed completely and desired mass was detected. The reaction mixture was poured into H₂O (20 mL) and extracted with DCM (20 mL×3). The combined organic layers were dried over Na₂SO₄, filtered, and concentrated under reduced pressure to give a residue. The reaction was purified by reversed-phase chromatography (40-60% (water (FA)-MeCN)). After reversed-phase purification, the eluent was concentrated and evaporated to remove organic solvents. The residual aqueous solution was lyophilized to give 1-cyclopropyl-5-((2R,6R)-4-(6,7-dimethyl-4-(3-(trifluoromethyl) bicyclo[1.1.1]pentan-1-yl) pteridin-2-yl)-6-methyltetrahydro-2H-pyran-2-yl)pyridin-2 (1H)-one (150 mg) as a solid, which was purified by SFC (Condition 4, Gradient b) to give 1-cyclopropyl-5-[(2R,4S,6R)-4-[6,7-dimethyl-4-[3-(trifluoromethyl)-1-bicyclo[1.1.1]pentanyl]pteridin-2-yl]-6-methyl-tetrahydropyran-2-yl]pyridin-2-one (Example 102, 74 mg, 0.137 mmol, 29% yield) as a solid. LCMS: [M+H]⁺=526.2; purity=98% (UV 220 nm); Ret. time=0.608 min. ¹H NMR (400 MHz, CDCl₃) δ 0.81-0.94 (m, 2H), 1.04-1.20 (m, 2H), 1.34 (d, J=6.1 Hz, 3H), 1.70-1.86 (m, 1H), 1.90-2.02 (m, 1H), 2.12-2.30 (m, 2H), 2.65 (s, 6H), 2.80 (d, J=14.4 Hz, 6H), 3.26-3.35 (m, 1H), 3.40-3.50 (m, 1H), 3.84 (ddd, J=11.1, 6.1, 1.9 Hz, 1H), 4.37 (dd, J=11.4, 1.8 Hz, 1H), 6.55 (d, J=9.3 Hz, 1H), 7.36-7.43 (m, 2H).

An analogous method was followed to obtain the following compounds.

| Compound No. | Starting Material | Characterization |
|---|---|---|
| 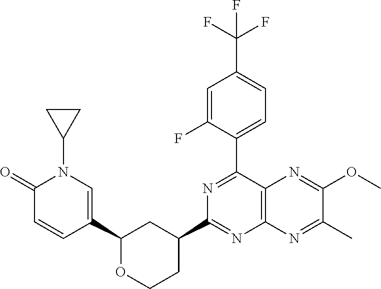 [SMILES: COc1nc2c(-c3ccc(C(F)(F)F)cc3F)nc([C@H]3CCO[C@@H](c4ccc(=O)n(C5CC5)c4)C3)nc2nc1C] | 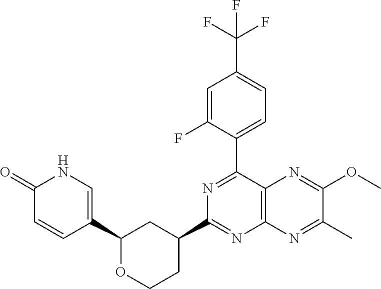 | The crude was then purified by Prep-HPLC (30-70% [water (FA)-ACN], 10 min) and lyophilized to give 1- cyclopropyl-5-[(2R,4S)-4-[4- [2-fluoro-4-(trifluoromethyl) phenyl]-6-methoxy-7- methyl-pteridin-2- yl]tetrahydropyran-2- yl]pyridin-2-one (Example 134, 40 mg, 0.07 mmol, 27% yield) as a solid. LCMS: [M + H]⁺ = 556.3; Purity = 100% (220 nm); Ret. time = 0.567 min. ¹H NMR (400 MHz, CDCl₃) δ 7.87 (t, J = 7.3 Hz, 1H), 7.62 (d, J = 7.9 Hz, 1H), 7.52 (d, J = 9.6 Hz, |
| | | 1H), 7.41-7.35 (m, 2H), 6.59- |
| Example 134 | | 6.51 (m, 1H), 4.38-4.24 (m, |
| | | 2H), 4.01 (s, 3H), 3.86-3.72 |
| | | (m, 1H), 3.60-3.46 (m, 1H), |
| | | 3.38-3.26 (m, 1H), 2.77 (s, |
| | | 3H), 2.34 (br d, J = 13.4 Hz, |
| | | 1H), 2.24-2.15 (m, 2H), 2.13- |
| | | 2.00 (m, 1H), 1.18-1.06 (m, |
| | | 2H), 0.94-0.82 (m, 2H). SFC |
| | | ee: 100%. |
| 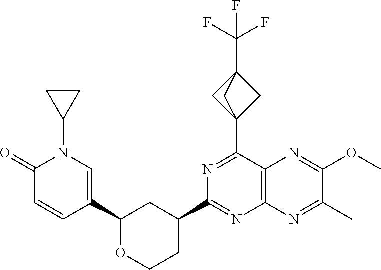 [SMILES: COc1nc2c(C34CC(C(F)(F)F)(C3)C4)nc([C@H]3CCO[C@@H](c4ccc(=O)n(C5CC5)c4)C3)nc2nc1C] | 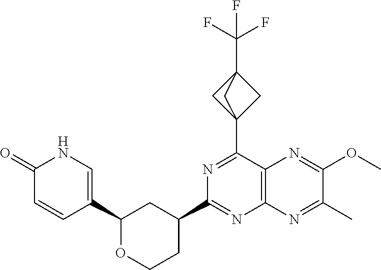 | Modification: The crude was purified by reversed-phase HPLC (0-100% [(0.05% FA)water/ ACN]) and lyophilized to give 1- cyclopropyl-5-[(2R,4S)-4-[6- methoxy-7-methyl-4-[3- (trifluoromethyl)-1-bicyclo- [1.1.1]pentanyl]pteridin-2- yl]tetrahydropyran-2- yl]pyridin-2-one (Example 142, 28 mg, 0.0511 mmol, 34% yield) as a solid. LCMS: [M + H]⁺ = 528.3; purity = 97% (UV 220 nm); Ret. time = 0.610 min. ¹H NMR (400 MHz, CDCl₃) δ 7.42-7.33 |
| | | (m, 2H), 6.54 (d, J = 10.1 Hz, |
| Example 142 | Example 143 | 1H), 4.38-4.22 (m, 2H), 4.14 |
| | | (s, 3H), 3.85-3.71 (m, 1H), |
| | | 3.48-3.36 (m, 1H), 3.36- |
| | | 3.27 (m, 1H), 2.73 (s, 3H), |
| | | 2.63 (s, 6H), 2.31-2.23 (m, |
| | | 1H), 2.17-2.07 (m, 2H), 2.05- |
| | | 1.93 (m, 1H), 1.17-1.09 (m, |
| | | 2H), 0.95-0.84 (m, 2H). |
| | | SFC: 98% purity. |
| 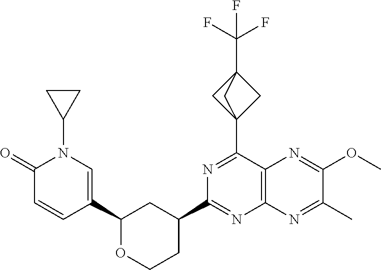 | 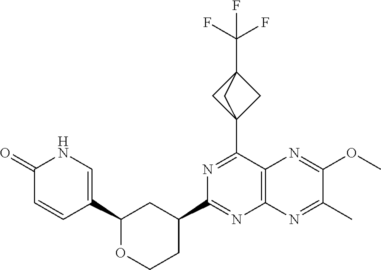 | Modification: The crude was run for 1 h. The crude was purified by prep-HPLC (Condition 18, Gradient c) and lyophilized to give 1- cyclopropyl-5-[(2R,4S)-4-[4- (4,4-difluorocyclohexyl)-6,7- dimethyl-pteridin-2- yl]tetrahydropyran-2- yl]pyridin-2-one (Example 225, single enantiomer of known absolute configuration, 79 mg, 0.159 mmol, 48% yield) as a solid. LCMS: [M + H]⁺ = 496.2. ¹H NMR (400 MHz, CDCl₃) δ 0.86-0.94 (m, 2H) 1.10-1.18 |
| | | (m, 2H) 1.94-2.20 (m, 9H) |
| Example 225 | Example 224 | 2.24-2.37 (m, 3H) 2.80 (d, |
| | | J = 15.0 Hz, 6H) 3.27-3.37 |
| | 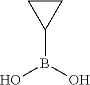 | (m, 1H) 3.40-3.52 (m, 1H) 3.79 (td, J = 11.3, 3.7 Hz, 1H) 4.17 (br t, J = 11.8 Hz, 1H) 4.25-4.37 (m, 2H) 6.52-6.60 (m, 1H) 7.38 (dq, J = 4.9, 2.4 Hz, 2H). ee ~ 92% |

### Method 6: Synthesis of Example 28-30

### Example 30:5-(4-(4-(4,4-difluorocyclohexyl)-6,7-dimethylpteridin-2-yl)tetrahydro-2H-pyran-2-yl)-1-methylpyridin-2 (1H)-one

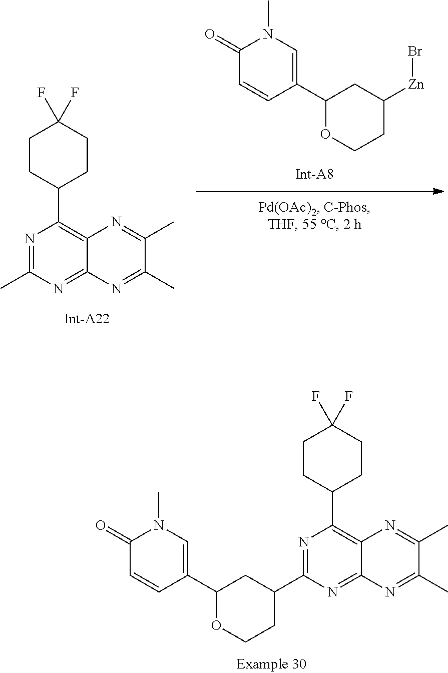

To a solution of 2-chloro-4-(4,4-difluorocyclohexyl)-6,7-dimethylpteridine (Int-A22, 1 equiv., 10 mg, 0.027 mmol), Pd(OAc)₂ (0.1 equiv., 0.60 mg, 0.003 mmol) and C-Phos (0.2 equiv., 2.3 mg, 0.005 mmol) in THF (1 mL) was purged with N₂ for 3 times, then (2-(1-methyl-6-oxo-1,6-dihydropyridin-3-yl)tetrahydro-2H-pyran-4-yl) zinc (II) bromide (Int-A8, 1.2 equiv., 12 mg, 0.032 mmol) was added, the reaction solution was stirred at 55° C. for 2 h. LCMS showed ~32% of desired product was detected. The mixture was quenched by 150 mL H₂O, extracted with DCM (100 mL×3), the combined organic layers was dried over anhydrous Na₂SO₄, filtered, and concentrated under reduced pressure to give the residue. The residue was purified by silica gel column chromatography (0-100% EtOAc in PE, then 0-50% MeOH in EtOAc)(TLC, 5% MeOH in DCM, desired product R_{f}=0.3) to give the 5-(4-(4-(4,4-difluorocyclohexyl)-6,7-dimethylpteridin-2-yl)tetrahydro-2H-pyran-2-yl)-1-methylpyridin-2 (1H)-one (Example 30, 150 mg, 0.291 mmol, 61% yield) as a solid. LCMS: [M+H]⁺=470.1.

### Isolation of Example 28-29

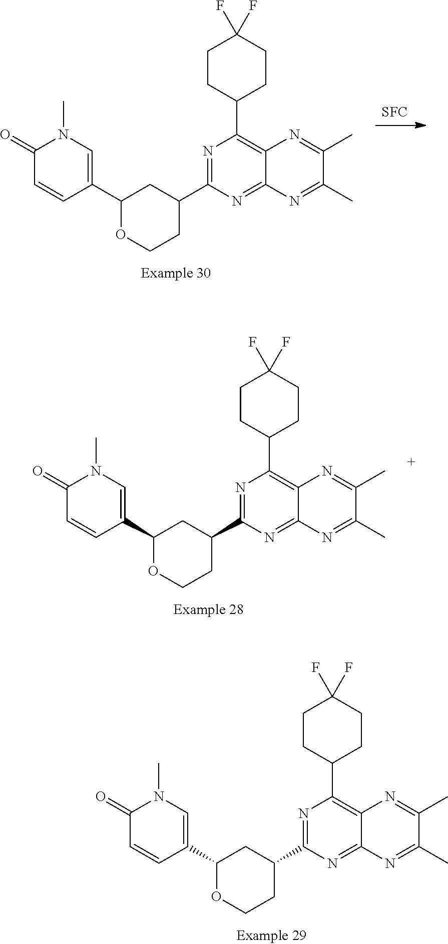

5-[4-[4-(4,4-difluorocyclohexyl)-6,7-dimethyl-pteridin-2-yl]tetrahydropyran-2-yl]-1-methyl-pyridin-2-one (Example 30, 1 equiv., 150 mg, 0.319 mmol) was purified by SFC (Condition 4, Gradient b) to give 25 mg Crude A and 70 mg Crude B.

25 mg Crude A was lyophilized to give 5-((2R,4S)-4-(4-(4,4-difluorocyclohexyl)-6,7-dimethylpteridin-2-yl)tetrahydro-2H-pyran-2-yl)-1-methylpyridin-2 (1H)-one (Example 28, 21 mg, 0.043 mmol, 13% yield) as a solid. LCMS: [M+H]⁺=470.3; purity=98% (220 nm); Ret. time=0.526 min. ¹H NMR (400 MHz, CDCl₃) δ 7.44-7.37 (m, 2H), 6.58 (d, J=10.1 Hz, 1H), 4.37-4.25 (m, 2H), 4.23-4.13 (m, 1H), 3.80 (dt, J=2.9, 11.8 Hz, 1H), 3.56 (s, 3H), 3.45 (tt, J=4.0, 11.8 Hz, 1H), 2.81 (d, J=15.5 Hz, 6H), 2.38-2.18 (m, 4H), 2.15-1.96 (m, 8H).

5-[4-[4-(4,4-difluorocyclohexyl)-6,7-dimethyl-pteridin-2-yl]tetrahydropyran-2-yl]-1-methyl-pyridin-2-one (Crude B, 1 equiv., 70 mg, 0.149 mmol) was purified by SFC (Condition Gradient a) to give 5-((2S,4R)-4-(4-(4,4-difluorocyclohexyl)-6,7-dimethylpteridin-2-yl)tetrahydro-2H-pyran-2-yl)-1-methylpyridin-2 (1H)-one (Example 29, 16 mg, 0.0314 mmol, 21% yield) as a solid. LCMS: [M+H]⁺=470.3; purity=90% (220 nm); Ret. time=0.529 min. ¹H NMR (400 MHz, CDCl₃) δ 7.46-7.37 (m, 2H), 6.61-6.54 (m, 1H), 4.38-4.26 (m, 2H), 4.22-4.13 (m, 1H), 3.80 (dt, J=2.8, 11.7 Hz, 1H), 3.56 (s, 3H), 3.45 (tt, J=4.1, 11.6 Hz, 1H), 2.83 (s, 3H), 2.79 (s, 3H), 2.36-2.19 (m, 4H), 2.17-1.99 (m, 8H).

An analogous method was followed to obtain the following compounds:

| Structure | Starting Materials | Characterization |
|---|---|---|
| 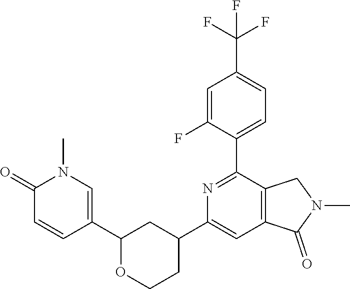 [SMILES: CN1Cc2c(cc(C3CCOC(c4ccc(=O)n(C)c4)C3)nc2-c2ccc(C(F)(F)F)cc2F)C1=O] | 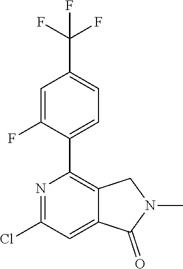 | The crude was purified by prep-TLC (EtOAc/MeOH = 10:1) to give 4-(2-fluoro-4- (trifluoromethyl)phenyl)-2-methyl-6-(2-(1- methyl-6-oxo-1,6-dihydropyridin-3- yl)tetrahydro-2H-pyran-4-yl)-2,3-dihydro-1H- pyrrolo[3,4-c]pyridin-1-one (Example 166, 30 mg, 0.0598 mmol, 21% yield) as an oil. ESI- MS: [M + H]⁺ = 502.2. Example 166 was then purified by SFC (Condition 18, Gradient a) to afford 15 mg peak 1 and 10 mg peak 2. 15 mg of peak 1 was purified by SFC (Condition 19, Gradient a) to give 4-(2-fluoro-4- (trifluoromethyl)phenyl)-2-methyl-6-((2S,4R)- 2-(1-methyl-6-oxo-1,6-dihydropyridin-3- yl)tetrahydro-2H-pyran-4-yl)-2,3-dihydro-1H- pyrrolo[3,4-c]pyridin-1-one (Example 342, 5.9 mg, 0.0116 mmol, 19% yield) as a solid. LCMS: [M + H]⁺ = 502.1. ¹H NMR (400 MHz, |
| | Int-E45 | CDCl₃) δ 7.85-7.69 (m, 2H), 7.64-7.56 (m, |
| Example 166 | | 1H), 7.54-7.47 (m, 1H), 7.40-7.33 (m, 2H), |
| Then, SFC- | | 6.60-6.55 (m, 1H), 4.45 (s, 2H), 4.35-4.26 |
| | | (m, 2H), 3.85-3.71 (m, 1H), 3.58-3.51 (m, |
| 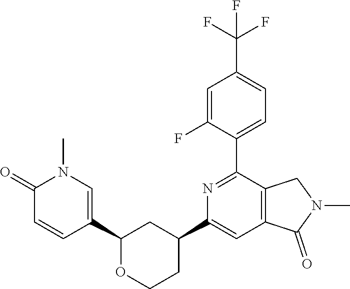 [SMILES: CN1Cc2c(cc([C@H]3CCO[C@@H](c4ccc(=O)n(C)c4)C3)nc2-c2ccc(C(F)(F)F)cc2F)C1=O] | 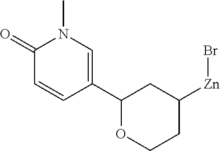 | 3H), 3.36-3.27 (m, 1H), 3.24 (s, 3H), 2.21 (br d, J = 13.3 Hz, 1H), 2.07-2.00 (m, 2H), 1.91- 1.82 (m, 1H). 10 mg of peak 2 was lyophilized to afford 4-(2- fluoro-4-(trifluoromethyl)phenyl)-2-methyl-6- ((2R,4S)-2-(1-methyl-6-oxo-1,6- dihydropyridin-3-yl)tetrahydro-2H-pyran-4- yl)-2,3-dihydro-1H-pyrrolo[3,4-c]pyridin-1- one (Example 165, 5.1 mg, 0.0010 mmol, 17% yield) as a solid. LCMS: [M + H]⁺ = 502.1. ¹H NMR (400 MHz, CDCl₃) δ 7.84-7.78 (m, 1H), 7.72 (s, 1H), 7.60 (d, J = 8.3 Hz, 1H), 7.53- 7.47 (m, 1H), 7.40-7.32 (m, 2H), 6.60-6.54 (m, 1H), 4.49-4.41 (m, 2H), 4.37-4.23 (m, 2H), 3.83-3.73 (m, 1H), 3.61-3.46 (m, 3H), 3.36-3.27 (m, 1H), 3.27-3.18 (m, 3H), 2.25- 2.16 (m, 1H), 2.09-1.99 (m, 2H), 1.91-1.79 (m, 1H). |
| Example 165 | | |
| 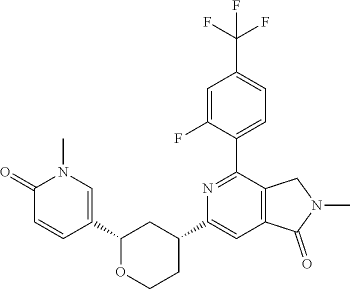 [SMILES: CN1Cc2c(cc([C@@H]3CCO[C@H](c4ccc(=O)n(C)c4)C3)nc2-c2ccc(C(F)(F)F)cc2F)C1=O] | | |
| Example 342 | | |

### Method 7: Synthesis of Example 38, 40, 42, 43, 44, 46, 48, 50, and 52

### Example 38:5-[(2R,4S)-4-[4-[3-(1,1-difluoroethyl)-1-bicyclo[1.1.1]pentanyl]-6,7-dimethyl-pteridin-2-yl]tetrahydropyran-2-yl]-1-methyl-pyridin-2-one

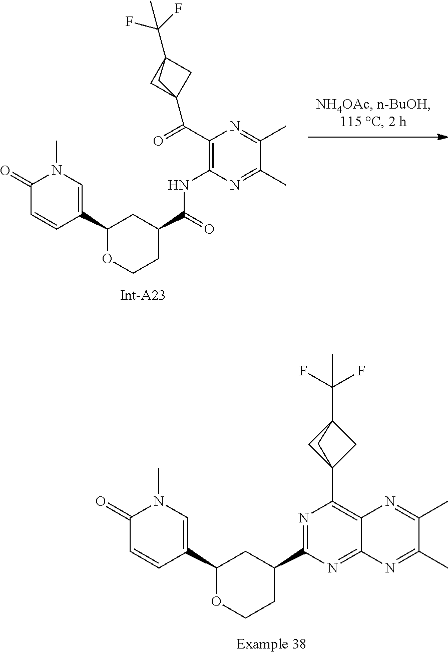 [SMILES: Cc1nc(NC(=O)[C@H]2CCO[C@@H](c3ccc(=O)n(C)c3)C2)c(C(=O)C23CC(C(C)(F)F)(C2)C3)nc1C ; "NH4OAc, n-BuOH,115 °C, 2 h" ; Cc1nc2nc([C@H]3CCO[C@@H](c4ccc(=O)n(C)c4)C3)nc(C34CC(C(C)(F)F)(C3)C4)c2nc1C]

To a solution of (2R,4S)—N-[3-[3-(1,1-difluoroethyl) bicyclo[1.1.1]pentane-1-carbonyl]-5,6-dimethyl-pyrazin-2-yl]-2-(1-methyl-6-oxo-3-pyridyl)tetrahydropyran-4-carboxamide (Int-A23, 1 equiv., 350 mg, 0.699 mmol) in 1-butanol (15 mL) was added ammonium acetate (20 equiv., 1078 mg, 14.0 mmol). Then the reaction mixture was stirred at 115° C. for 2 hours. LCMS showed starting material was consumed completely and desired mass was detected. The reaction mixture was diluted with water 10 mL and extracted with EtOAc (20 mL×2). The combined organic layers were dried over Na₂SO₄, filtered, and concentrated under reduced pressure to give a crude residue. The residue was purified by silica gel column chromatography (0-100% EtOAc in PE)(TLC, 5% MeOH in DCM, R_{f}=0.4) to give the crude product (200 mg). The crude product was purified by SFC (Condition 13, Gradient a) to give 5-[(2R,4S)-4-[4-[3-(1,1-difluoroethyl)-1-bicyclo[1.1.1]pentanyl]-6,7-dimethyl-pteridin-2-yl]tetrahydropyran-2-yl]-1-methyl-pyridin-2-one (Example 38, 114 mg, 0.237 mmol, 34% yield) as a solid. LCMS: [M+H]⁺=482.1; purity=99% (220 nm); Ret. time=0.553 min. ¹H NMR (400 MHz, CDCl₃) δ 1.66-1.71 (m, 3H), 1.97-2.08 (m, 1H), 2.08-2.21 (m, 2H), 2.32 (br d, J=13.1 Hz, 1H), 2.53 (s, 6H), 2.79 (d, J=13.6 Hz, 6H), 3.40-3.50 (m, 1H), 3.56 (s, 3H), 3.80 (td, J=11.4, 3.3 Hz, 1H), 4.25-4.37 (m, 2H), 6.59 (dd, J=8.5, 1.5 Hz, 1H), 7.37-7.46 (m, 2H).

An analogous procedure was followed to obtain the following compounds.

| Structure | Intermediate | Characterization |
|---|---|---|
| 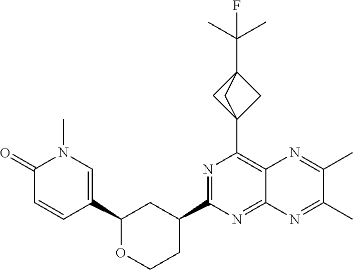 [SMILES: Cc1nc2nc([C@H]3CCO[C@@H](c4ccc(=O)n(C)c4)C3)nc(C34CC(C(C)(C)F)(C3)C4)c2nc1C] | 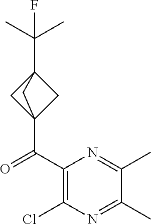 | The crude was purified by reversed- phase chromatography (40% [water (FA)-ACN]) and lyophilized to give 5- [(2R,4S)-4-[4-[3-(1-fluoro-1-methyl- ethyl)-1-bicyclo[1.1.1]pentanyl]-6,7- dimethyl-pteridin-2-yl]tetrahydropyran- 2-yl]-1-methyl-pyridin-2-one (Example 52, 6.0 mg, 0.012 mmol, 12% yield) as a solid. LCMS: [M + H]⁺ = 478.3; purity = 96% (UV 220 nm); Ret. time = 0.573 min. ¹H NMR (400 MHz, CDCl₃) δ 7.43- 7.36 (m, 2H), 6.60-6.53 (m, 1H), 4.35- 4.24 (m, 2H), 3.85-3.69 (m, 1H), 3.55 (s, 3H), 3.49-3.39 (m, 1H), 2.79 (s, 3H), 2.76 (s, 3H), 2.42-2.39 (m, 6H), 2.33 (br d, J = 1.0 Hz, 1H), 2.12 (br s, 2H), 2.05-2.00 (m, 1H), 1.45-1.42 (m, 3H), |
| | | 1.39-1.37 (m, 3H). SFC: 95.7% ce. |
| Example 52 | Int-A27 | |
| 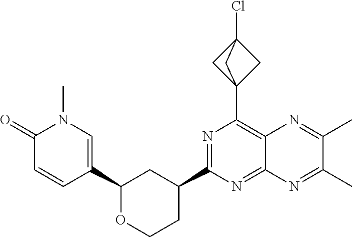 [SMILES: Cc1nc2nc([C@H]3CCO[C@@H](c4ccc(=O)n(C)c4)C3)nc(C34CC(Cl)(C3)C4)c2nc1C] | 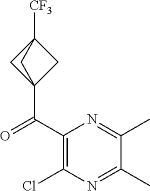 [SMILES: Cc1nc(Cl)c(C(=O)C23CC(C(F)(F)F)(C2)C3)nc1C] | The residue was purified by flash column (100% EtOAc, R_{f} = 0.3) and concentrated under reduced pressure to give a 5-[(2R,4S)-4-[4-(3-chloro-1- bicyclo[1.1.1]-pentanyl)-6,7-dimethyl- pteridin-2-yl]tetrahydropyran-2-yl]-1- methyl-pyridin-2-one (160 mg, 0.342 mmol, 90% yield) as a solid. 160 mg product was separated by SFC (Condition 6, Gradient f) and lyophilized to give two peaks (peak 1 and peak 2). Peak 2: 5-[(2R,4S)-4-[4-(3-chloro-1- bicyclo[1.1.1]pentanyl)-6,7-dimethyl- pteridin-2-yl]tetrahydropyran-2-yl]-1- methyl-pyridin-2-one (Example 40, 82 |
| | | mg, 0.178 mmol, 50% yield) as a solid. |
| Example 40 | | LCMS: [M + H]⁺ = 452.2; purity = 99% |
| | | (UV 220 nm); Ret. time = 0.563 min. 1H |
| | | NMR (400 MHz, CDCl₃) δ 7.42-7.36 |
| | | (m, 2H), 6.59-6.52 (m, 1H), 4.41-4.17 |
| | | (m, 2H), 3.78 (dt, J = 3.6, 11.4 Hz, 1H), |
| | | 3.54 (s, 3H), 3.43 (br t, J = 4.2 Hz, 1H), |
| | | 2.85-2.78 (m, 9H), 2.78-2.74 (m, 3H), |
| | | 2.30 (br dd, J = 1.9, 13.3 Hz, 1H), 2.18- |
| | | 2.07 (m, 2H), 2.06-1.96 (m, 1H). SFC: |
| | | 98.7% ee. |
| 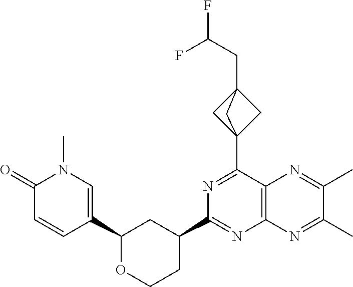 [SMILES: Cc1nc2nc([C@H]3CCO[C@@H](c4ccc(=O)n(C)c4)C3)nc(C34CC(CC(F)F)(C3)C4)c2nc1C] | 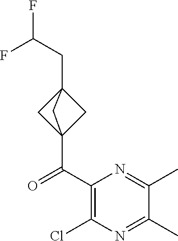 | The crude was purified by reversed- phase flash (0.1% FA) to afford 5- [(2R,4S)-4-[4-[3-(2,2-difluoroethyl)-1- bicyclo[1.1.1] pentanyl]-6,7-dimethyl- pteridin-2-yl]tetrahydropyran-2-yl]-1- methyl-pyridin-2-one (Example 46, 14 mg, 0.029 mmol, 72% yield) as a solid. LCMS: [M + H]⁺ = 482.2; purity = 99% (UV 220 nm); Ret. time = 0.541 min. SFC: 99% purity. ¹H NMR (400 MHz, CDCl₃) δ 7.38 (s, 1H), 7.42-7.37 (m, 1H), 6.56 (d, J = 9.6 Hz, 1H), 5.91 (tt, J = 4.6, 56.4 Hz, 1H), 4.35-4.24 (m, 2H), 3.78 (dt, J = 3.7, 11.4 Hz, 1H), 3.55 (s, 3H), 3.48-3.37 (m, 1H), 2.77 (d, J = 12.6 Hz, 6H), 2.46 (s, 6H), 2.34-2.26 (m, 1H), 2.09 (br d, J = 4.4 Hz, 4H), 2.08- 1.96 (m, 1H). |
| Example 46 | Int-A34 | |
| 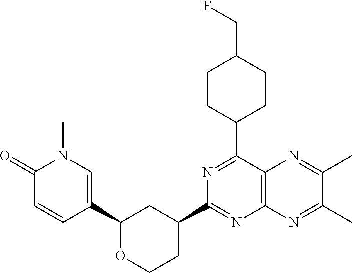 [SMILES: Cc1nc2nc([C@H]3CCO[C@@H](c4ccc(=O)n(C)c4)C3)nc(C3CCC(CF)CC3)c2nc1C] | 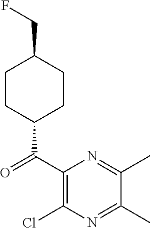 [SMILES: Cc1nc(Cl)c(C(=O)[C@H]2CC[C@H](CF)CC2)nc1C] | The crude product was purified by reversed-phase HPLC (40-60% [(0.05% FA)H₂O-ACN] and lyophilized to give Crude A 5-[(2R,4S)-4-[4-[4- (fluoromethyl)cyclohexyl]-6,7- dimethyl-pteridin-2-yl]tetrahydropyran- 2-yl]-1-methyl-pyridin-2-one (14 mg, 0.03 mmol, 11% yield) as a solid, Crude B 5-[(2R,4S)-4-[4-[4-(fluoromethyl)- cyclohexyl]-6,7-dimethyl-pteridin-2- yl]tetrahydropyran-2-yl]-1-methyl- pyridin-2-one (32 mg, 0.07 mmol, 26% yield) as a solid, pure 5-[(2R,4S)-4-[4- [4-(fluoromethyl)-cyclohexyl]-6,7- dimethyl-pteridin-2-yl]tetrahydropyran- 2-yl]-1-methyl-pyridin-2-one (Example 50, 9.1 mg, 0.019 mmol, 7% yield) as a |
| | | solid. LCMS: [M + H]⁺ = 466.2, purity = |
| Example 50 | | 97% (220 nm); Ret. time = 0.548 min. |
| | | ¹H NMR (400 MHz, CDCl₃) δ 7.44- |
| | | 7.37 (m, 2H), 6.61-6.55 (m, 1H), 4.41 |
| | | (d, J = 5.8 Hz, 1H), 4.36-4.27 (m, 3H), |
| | | 4.14-4.04 (m, 1H), 3.80 (dt, J = 3.3, |
| | | 11.7 Hz, 1H), 3.57-3.54 (m, 3H), 3.50- |
| | | 3.41 (m, 1H), 2.80 (d, J = 13.4 Hz, 6H), |
| | | 2.38-2.31 (m, 1H), 2.21-2.09 (m, 2H), |
| | | 2.05-1.94 (m, 5H), 1.92-1.81 (m, 3H), |
| | | 1.44-1.30 (m, 2H). ¹⁹F NMR (376 |
| | | MHz, CDCl₃) δ −223.02 (s, 1F). |
| 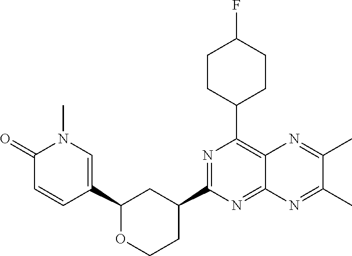 [SMILES: Cc1nc2nc([C@H]3CCO[C@@H](c4ccc(=O)n(C)c4)C3)nc(C3CCC(F)CC3)c2nc1C] | 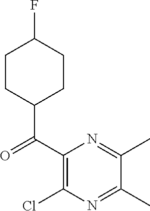 [SMILES: Cc1nc(Cl)c(C(=O)C2CCC(F)CC2)nc1C] | Example 44: The residue was purified by Prep-TLC (6% MeOH in DCM, TLC, desired product R_{f} = 0.5) to give a crude product which was further purified by SFC (Condition 1, Gradient a-1) and lyophilized to give the crude product. SFC showed ee ~30%. The crude product was purified again by SFC (Condition 4, Gradient b) (x2) and lyophilized to give 5-((2R,4S)-4-(4-(4-fluorocyclohexyl)- 6,7-dimethylpteridin-2-yl)tetrahydro- 2H-pyran-2-yl)-1-methylpyridin-2(1H)- one (Example 44). SFC showed ee ~ 99%. Then, SFC- Example 44 (1.14 equiv., 80 mg, 0.177 mmol) was purified by Prep-HPLC |
| | | (Condition 7, Gradient b) to give 5- |
| Example 44 | | ((2R,4S)-4-(4-((1s,4R)-4- |
| Then, SFC- | | fluorocyclohexyl)-6,7-dimethylpteridin- |
| | | 2-yl)tetrahydro-2H-pyran-2-yl)-1- |
| 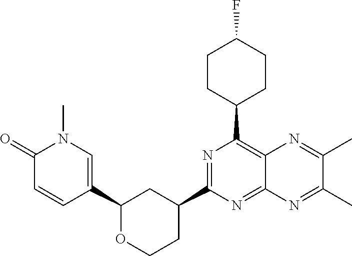 [SMILES: Cc1nc2nc([C@H]3CCO[C@@H](c4ccc(=O)n(C)c4)C3)nc([C@H]3CC[C@H](F)CC3)c2nc1C] | | methylpyridin-2(1H)-one (Example 43, 19 mg, 0.042 mmol, 27% yield) as a solid. Example 43: LCMS: [M + H]⁺ = 452.3; purity = 100%; Ret. time = 0.510 min. HPLC: Ret. time = 1.346 min, 99.8% purity at 220 nm. ¹H NMR (400 MHz, CDCl₃) δ 7.44-7.36 (m, 2H), 6.57 (d, J = 10.3 Hz, 1H), 5.08-4.88 (m, 1H), 4.30 (br s, 2H), 4.18-4.06 (m, 1H), 3.78 (dt, J = 2.9, 11.6 Hz, 1H), 3.55 (s, 3H), 3.50-3.38 (m, 1H), 2.78 (d, J = 13.9 Hz, 6H), 2.33 (br d, J = 13.3 Hz, 1H), 2.27-2.07 (m, 6H), 2.06-1.94 (m, 1H), 1.90-1.82 (m, 1H), 1.78 (br d, J = 10.1 Hz, 3H). 5-((2R,4S)-4-(4-((1r,4S)-4- |
| | | fluorocyclohexyl)-6,7-dimethyl- |
| Example 42 | | pteridin-2-yl)tetrahydro-2H-pyran-2- |
| | | yl)-1-methylpyridin-2(1H)-one (1 |
| 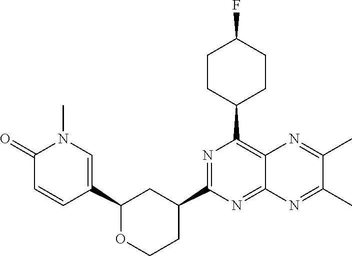 [SMILES: Cc1nc2nc([C@H]3CCO[C@@H](c4ccc(=O)n(C)c4)C3)nc([C@H]3CC[C@@H](F)CC3)c2nc1C] | | equiv., 70 mg, 0.155 mmol) was purified by Prep-HPLC (Condition 7, Gradient a) to give 5-((2R,4S)-4-(4-((1r,4S)-4- fluorocyclohexyl)-6,7-dimethyl- pteridin-2-yl)tetrahydro-2H-pyran-2- yl)-1-methylpyridin-2(1H)-one (Example 42, 12 mg, 0.0264 mmol, 17% yield) as a solid. Example 42: LCMS: [M + H]⁺ = 452.3; purity = 100% (UV 220 nm); Ret. time = 0.510 min. HPLC: Ret. time = 1.368 min, 99.5% purity at 220 nm. ¹H NMR (400 MHz, CDCl₃) δ 7.44-7.36 (m, 2H), 6.57 (d, J = 10.3 Hz, 1H), 4.79-4.55 (m, 1H), 4.30 (s, 2H), 4.14-4.02 (m, 1H), 3.78 (dt, J = 2.8, 11.7 Hz, 1H), 3.55 (s, 3H), 3.48-3.36 |
| | | (m, 1H), 2.79 (d, J = 11.5 Hz, 6H), 2.29 |
| Example 43 | | (br s, 3H), 2.13 (br d, J = 3.3 Hz, 2H), |
| | | 2.08-1.97 (m, 3H), 1.88 (br d, J = 12.4 |
| | | Hz, 2H), 1.83-1.76 (m, 2H). |
| 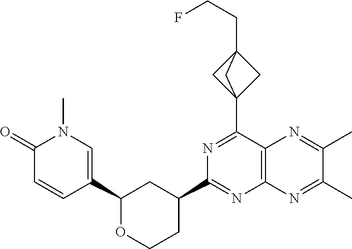 [SMILES: Cc1nc2nc([C@H]3CCO[C@@H](c4ccc(=O)n(C)c4)C3)nc(C34CC(CCF)(C3)C4)c2nc1C] | 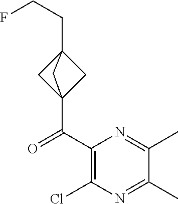 | The crude product was purified by prep- TLC (DCM/MeOH = 10:1, Rf = 0.4) and lyophilized to give 5-((2R,4S)-4-(4-(3- (2-fluoroethyl)bicyclo[1.1.1]pentan-1- yl)-6,7-dimethylpteridin-2- yl)tetrahydro-2H-pyran-2-yl)-1- methylpyridin-2(1H)-one (Example 48, 42 mg, 0.0897 mmol, 62% yield) as a solid. LCMS: [M + H]⁺ = 464.4; purity = 98% (220 nm); Ret. time = 0.540 min. SFC showed ee is 89%. ¹H NMR (400 MHz, CDCl₃) δ 7.43-7.36 (m, 2H), 6.61- 6.53 (m, 1H), 4.68-4.51 (m, 2H), 4.36- 4.24 (m, 2H), 3.83-3.74 (m, 1H), 3.55 (s, 3H), 3.49-3.38 (m, 1H), 2.78 (d, J = |
| | | 11.6 Hz, 6H), 2.40 (s, 6H), 2.31 (br dd, |
| Example 48 | Int-A43 | J = 1.7, 13.2 Hz, 1H), 2.19-2.10 (m, 2H), |
| | | 2.10-2.04 (m, 2H), 2.04-1.96 (m, 2H). |
| 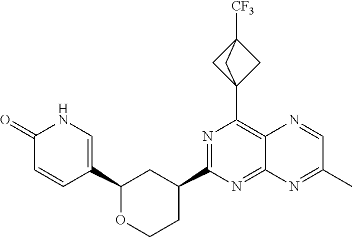 [SMILES: Cc1cnc2c(C34CC(C(F)(F)F)(C3)C4)nc([C@H]3CCO[C@@H](c4ccc(=O)[nH]c4)C3)nc2n1] | 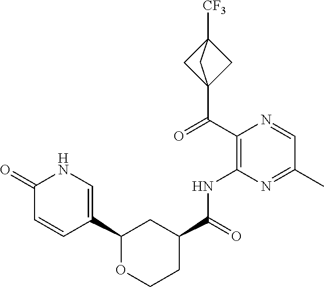 | The crude mixture was purified by silica gel flash-column chromatography using an elution gradient of 1%-7% MeOH in DCM to yield 5-((2R,4S)-4-(7-methyl-4- (3-(trifluoromethyl)bicyclo[1.1.1]pentan- 1-yl)pteridin-2-yl)tetrahydro-2H-pyran- 2-yl)pyridin-2(1H)-one (Example 187) (898 mg, 1.91 mmol, 94% yield) as a foam. ESI-MS: [M + H]⁺ = 458.4. ¹H NMR (DMSO-d₆, 400 MHz): δ 11.49 (1H, s), 9.00 (1H, s), 7.49 (1H, dd, J = 9.5, 2.6 Hz), 7.32 (1H, d, J = 2.5 Hz), 6.31 (1H, d, J = 9.4 Hz), 4.37 (1H, dd, J = 11.2, 2.0 Hz), 4.14 (1H, dd, J = 11.3, 4.2 Hz), 3.70 (1H, t, J = 11.7 Hz), 3.37- 3.45 (1H, m), 2.78 (3H, s), 2.62 (6H, s), 2.17 (1H, d, J = 12.8 Hz), 2.03 (1H, d, J = 13.2 Hz), 1.90 (1H, qd, J = 12.5, 4.6 Hz), 1.79 (1H, dd, J = 24.2, 12.0 Hz). ¹⁹F NMR (DMSO-d₆, 376 MHz): δ −71.5 (3F, s). |
| Example 187 | Int-C24 | |
| 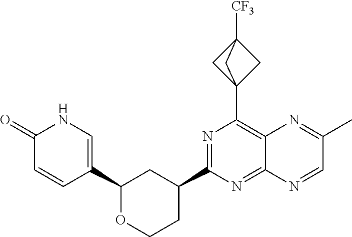 [SMILES: Cc1cnc2nc([C@H]3CCO[C@@H](c4ccc(=O)[nH]c4)C3)nc(C34CC(C(F)(F)F)(C3)C4)c2n1] | 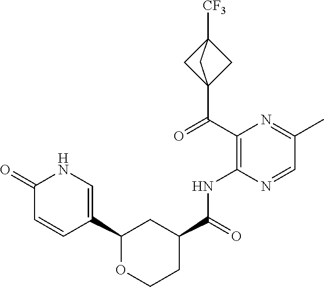 | Modifications: The crude mixture was purified by silica gel flash-column chromatography using an elution gradient of 1%-7% MeOH in DCM to yield 5-((2R,4S)-4-(6-methyl-4-(3- (trifluoromethyl)bicyclo[1.1.1]pentan- 1-yl)pteridin-2-yl)tetrahydro-2H-pyran- 2-yl)pyridin-2(1H)-one (Example 197) (1.03 g, 2.03 mmol, 83% yield) as a foam. ESI-MS: [M + H]⁺ = 458.4. ¹H NMR (CDCl₃, 400 MHz): 89.01 (1H, s), 7.83 (1H, d, J = 9.2 Hz), 7.74 (1H, s), 6.87 (1H, d, J = 9.3 Hz), 4.47 (1H, d, J = 11.3 Hz), 4.29-4.33 (1H, m), 3.77-3.84 (1H, m), 3.47-3.53 (1H, m), 2.82 (3H, s), 2.66 (6H, s), 2.35 (1H, d, J = 14.1 Hz), 2.09-2.18 (3H, m), 1.94-2.03 (1H, m). ¹⁹F NMR (CDCl₃, 376 MHz): δ −73.1 (3F, s). |
| Example 197 | Int-C34 | |
| 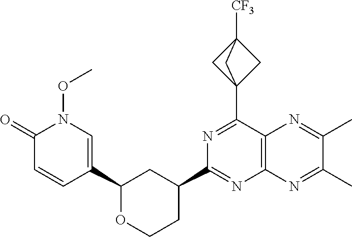 [SMILES: COn1cc([C@H]2C[C@@H](c3nc(C45CC(C(F)(F)F)(C4)C5)c4nc(C)c(C)nc4n3)CCO2)ccc1=O] | 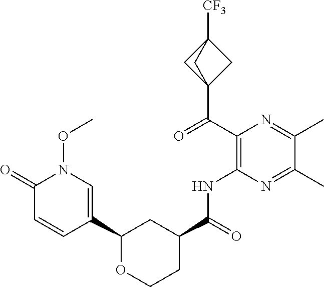 | Modifications: The reaction crude was purified, first, by normal phase flash chromatography using an elution gradient of 0-10% MeOH in DCM, and second, by prep-HPLC (Condition 16, Gradient b) to yield 5-[(2R,4S)-4-[6,7- dimethyl-4-[3-(trifluoromethyl)-1- bicyclo[1.1.1]pentanyl]pteridin-2- yl]tetrahydropyran-2-yl]-1-methoxy- pyridin-2-one (Example 203, 21 mg, 0.036 mmol, 38% yield) as a solid. ESI- MS: [M + H]⁺ = 502.4. ¹H NMR (DMSO-d₆, 400 MHz): δ 7.89 (d, J = 2.5 Hz, 1H), 7.47 (dd, J = 9.5, 2.4 Hz, 1H), 6.49 (d, J = 9.6 Hz, 1H), 4.36 (dd, J = 11.0, 0.9 Hz, 1H), 4.12 (dd, J = 11.2, 3.4 Hz, 1H), 3.90 (s, 3H), 3.71-3.65 (m, 1H), 3.40-3.35 (m, 1H), 2.70 (s, 6H), 2.59 (s, |
| | | 6H), 2.20-2.17 (m, 1H), 2.01-1.98 (m, |
| Example 203 | Int-C45 | 1H), 1.93-1.82 (m, 1H), 1.76 (q, J = 12.1 |
| | | Hz, 1H). ¹⁹F NMR (DMSO-d₆, 376 |
| | | MHz): δ −71.3 (s, 3F). |
| 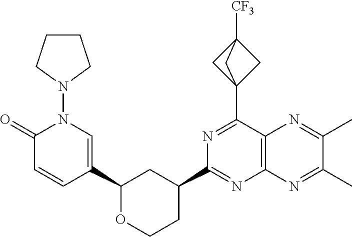 [SMILES: Cc1nc2nc([C@H]3CCO[C@@H](c4ccc(=O)n(N5CCCC5)c4)C3)nc(C34CC(C(F)(F)F)(C3)C4)c2nc1C] | 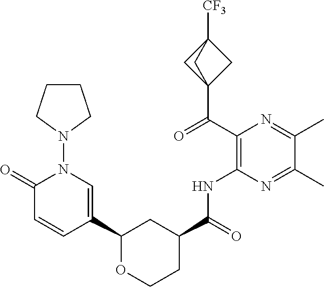 | Modifications: The reaction was run in ethanol (0.6 mL per mmol NH₄OAc) at 80 °C for 4 h. The reaction crude was purified by normal phase flash chromatography using an elution gradient of 10-50% EtOAc:EtOH (3:1) in hexanes to yield 5-[(2R,4S)-4-[6,7- dimethyl-4-[3-(trifluoromethyl)-1- bicyclo[1.1.1]pentanyl]pteridin-2- yl]tetrahydropyran-2-yl]-1-pyrrolidin-1- yl-pyridin-2-one (Example 209, 53 mg, 0.098 mmol, 57% yield) as a solid. ESI- MS: [M + H]⁺ = 541.4. ¹H NMR (CDCl₃, 400 MHz): δ δ 7.59 (d, J = 2.5 Hz, 1H), 7.39 (dd, J = 9.5, 2.6 Hz, 1H), 6.55 (d, J = 9.4 Hz, 1H), 4.31-4.25 (m, 2H), 3.82- 3.75 (m, 1H), 3.48-3.39 (m, 5H), 2.81 (s, 3H), 2.77 (s, 3H), 2.65 (s, 6H), 2.32-2.29 |
| | | (m, 1H), 2.17-2.01 (m, 3H), 1.98-1.93 |
| Example 209 | Int-D8 | (m, 4H). ¹⁹F NMR (CDCl₃, 376 MHz): |
| | | δ −72.9 (s, 3F). |
| 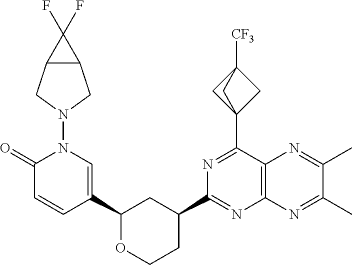 [SMILES: Cc1nc2nc([C@H]3CCO[C@@H](c4ccc(=O)n(N5CC6C(C5)C6(F)F)c4)C3)nc(C34CC(C(F)(F)F)(C3)C4)c2nc1C] | 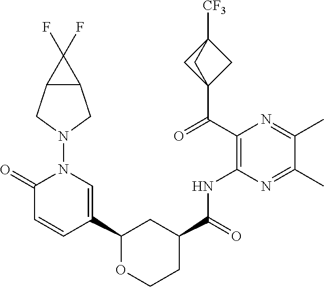 | Modification: The crude material was purified by prep-HPLC (Condition 10, Gradient d) to afford 1-(6,6-difluoro-3- azabicyclo[3.1.0]hexan-3-yl)-5- [(2R,4S)-4-[6,7-dimethyl-4-[3- (trifluoromethyl)-1- bicyclo[1.1.1]pentanyl]pteridin-2- yl]tetrahydropyran-2-yl]pyridin-2-one (Example 215, 4.8 mg, 8.2 μmol, 25% yield) as a solid. ESI-MS: [M + H]⁺ = 589.3. ¹H NMR (CDCl₃, 400 MHz): δ 7.51 (d, J = 2.5 Hz, 1H), 7.41 (dd, J = 9.5, 2.6 Hz, 1H), 6.55 (d, J = 9.4 Hz, 1H), 4.31-4.26 (m, 2H), 4.11-4.09 (m, 2H), 3.78 (td, J = 11.7, 2.7 Hz, 1H), 3.49 (d, J = 8.5 Hz, 2H), 3.46-3.39 (m, 1H), 2.81 (s, 3H), 2.77 (s, 3H), 2.64 (s, 6H), 2.32-2.27 (m, |
| | | 3H), 2.18-1.99 (m, 3H). |
| Example 215 | Int-D11 | ¹⁹F NMR (CDCl₃, 376 MHz): δ −73.0 (s, |
| | | 3F), −127.7 (dt, J = 158.3, 13.0 Hz, 1F), |
| | | −151.1 (d, J = 159.8 Hz, 1F). |
| 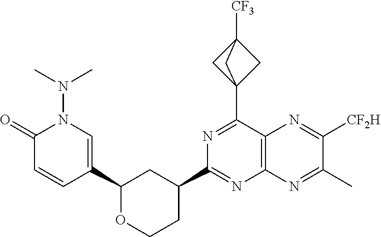 [SMILES: Cc1nc2nc([C@H]3CCO[C@@H](c4ccc(=O)n(N(C)C)c4)C3)nc(C34CC(C(F)(F)F)(C3)C4)c2nc1C(F)F] | 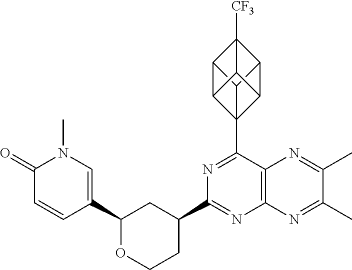 [SMILES: Cc1nc2nc([C@H]3CCO[C@@H](c4ccc(=O)n(C)c4)C3)nc(C34C5C6C3C3C4C5C63C(F)(F)F)c2nc1C] | Modifications: The reaction was run at 115 °C for 10 min. The crude material was purified by prep-HPLC (Condition 19, Gradient a) to give the desired product 5-((2R,4R)-4-(6- (difluoromethyl)-7-methyl-4-(3- (trifluoromethyl)bicyclo[1.1.1]pentan- 1-yl)pteridin-2-yl)tetrahydro-2H-pyran- 2-yl)-1-(dimethylamino)pyridin-2(1H)- one (Example 220, 3.0 mg, 5.45 μmol, 18% yield). ¹H NMR (400 MHz, DMSO-d₆) δ 7.54-7.52 (m, 1H), 7.44- 7.39 (m, 1H), 6.35 (d, J = 9.4 Hz, 1H), 4.35 (d, J = 10.1 Hz, 1H), 4.12 (dd, J = 11.3, 3.3 Hz, 1H), 3.68 (t, J = 10.9 Hz, 1H), 3.44-3.38 (m, 1H), 2.89 (s, 6H), 2.84 (d, J = 10.3 Hz, 3H), 2.63-2.61 (m, |
| | | 7H), 2.29 (t, J = 1.8 Hz, 1H), 2.18 (d, J = |
| Example 220 | Int-D17 | 12.6 Hz, 1H), 2.04-2.00 (m, 1H), 1.88 |
| | | (dd, J = 12.9, 4.2 Hz, 1H), 1.76 (dd, J = |
| | | 24.6, 11.8 Hz, 1H). ¹⁹F NMR (376 MHz, |
| | | DMSO-d₆) δ −71.3 (s, 3F), −119.0 (m, |
| | | 2F). ESI-MS: [M + H]⁺ = 551.4. |
| 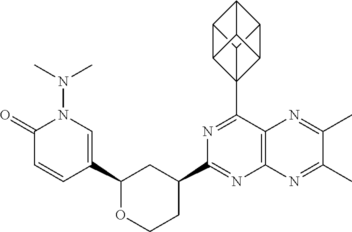 [SMILES: Cc1nc2nc([C@H]3CCO[C@@H](c4ccc(=O)n(N(C)C)c4)C3)nc(C34C5C6C7C5C3C7C64)c2nc1C] | 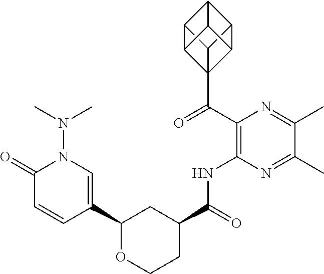 | Modifications: The reaction was run in EtOH at 60 °C for 36 h. The crude was purified by SiO₂ flash column chromatography using an elution gradient of 1-8% MeOH in DCM to yield 5-((2R,4S)-4-(4-(cuban-1-yl)-6,7- dimethylpteridin-2-yl)tetrahydro-2H- pyran-2-yl)-1-(dimethylamino)pyridin- 2(1H)-one (Example 231, 45 mg, 0.086 mmol, 74% yield ) as a solid. ESI-MS: [M + H]⁺ = 483.5. ¹H NMR (400 MHz, DMSO-d₆): δ 7.53 (d, J = 2.5 Hz, 1H), 7.42 (dd, J = 9.4, 2.5 Hz, 1H), 6.33 (d, J = 9.4 Hz, 1H), 4.47-4.44 (m, 3H), 4.32 (d, J = 10.1 Hz, 1H), 4.15-4.06 (m, 5H), 3.68-3.62 (m, 1H), 3.33 (dt, J = 12.0, 3.7 Hz, 1H), 2.89 (s, 6H), 2.70 (s, 3H), 2.66 |
| | | (s, 3H), 2.17 (d, J = 13.0 Hz, 1H), 2.00 |
| Example 231 | Int-D46 | (d, J = 11.7 Hz, 1H), 1.92-1.82 (m, 1H), |
| | | 1.75 (dd, J = 24.6, 12.0 Hz, 1H). |
| 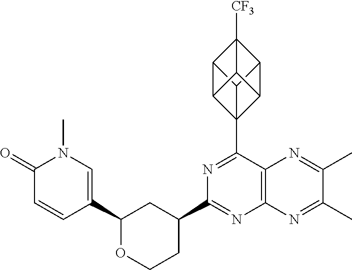 [SMILES: Cc1nc2nc([C@H]3CCO[C@@H](c4ccc(=O)n(C)c4)C3)nc(C34C5C6C3C3C4C5C63C(F)(F)F)c2nc1C] | 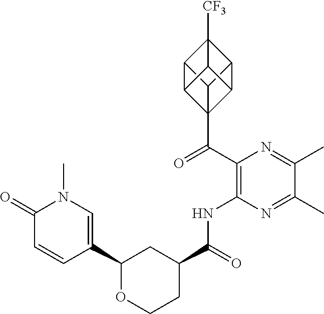 | Modifications: The reaction was run in EtOH at 60 °C for 36 h. The crude was purified by SiO₂ flash column chromatography elution gradient of 1-8% MeOH in DCM to yield 5-((2R,4S)-4-(6,7-dimethyl-4-(4- (trifluoromethyl)cuban-1-yl)pteridin-2- yl)tetrahydro-2H-pyran-2-yl)-1- methylpyridin-2(1H)-one (Example 233, 28 mg, 0.051 mmol, 65% yield ) as a solid. ESI-MS: [M + H]⁺ = 522.3. ¹H NMR (400 MHz, DMSO-d₆): δ 7.66 (d, J = 2.3 Hz, 1H), 7.44 (dd, J = 9.1, 2.5 Hz, 1H), 6.32 (d, J = 9.4 Hz, 1H), 4.48 (t, J = 4.9 Hz, 3H), 4.36 (t, J = 4.7 Hz, 3H), 4.31 (d, J = 10.1 Hz, 1H), 4.11 (dd, J = 11.2, 3.4 Hz, 1H), 3.70-3.65 (m, 1H), 3.44-3.33 (m, 4H), 2.70 (s, 3H), 2.68 (s, 3H), 2.18 |
| | | (d, J = 13.0 Hz, 1H), 2.01 (d, J = 11.4 Hz, |
| Example 233 | Int-D47 | 1H), 1.93-1.74 (m, 2H). ¹⁹F NMR (376 |
| | | MHz, DMSO-d₆): δ −75.7 (s, 3F). |
| 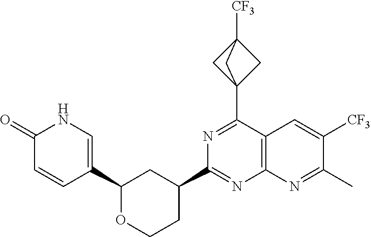 [SMILES: Cc1nc2nc([C@H]3CCO[C@@H](c4ccc(=O)[nH]c4)C3)nc(C34CC(C(F)(F)F)(C3)C4)c2cc1C(F)(F)F] | 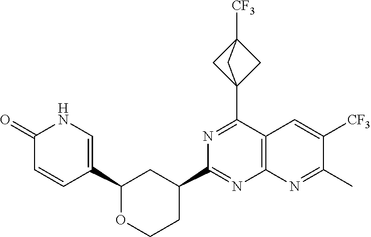 [SMILES: Cc1nc2nc([C@H]3CCO[C@@H](c4ccc(=O)[nH]c4)C3)nc(C34CC(C(F)(F)F)(C3)C4)c2cc1C(F)(F)F] | The crude mixture was purified by silica gel flash column chromatography (2- 10% MeOH in DCM) to yield 5- ((2R,4S)-4-(7-methyl-6- (trifluoromethyl)-4-(3- (trifluoromethyl)bicyclo[1.1.1]pentan- 1-yl)pyrido[2,3-d]pyrimidin-2- yl)tetrahydro-2H-pyran-2-yl)pyridin- 2(1H)-one (Example 330) as a foam. ESI-MS: [M + H]⁺ = 525.2. ¹H NMR (400 MHz, CD₃OD): δ 8.91 (s, 1H), 7.68 (dd, J = 9.4, 2.5 Hz, 1H), 7.45 (d, J = 2.3 Hz, 1H), 6.53 (d, J = 9.6 Hz, 1H), 4.44 (dd, J = 11.3, 1.7 Hz, 1H), 4.26-4.22 (m, 1H), 3.84-3.77 (m, 1H), 3.53-3.45 (m, |
| | | 1H), 2.89 (s, 3H), 2.68 (s, 6H), 2.27 (d, |
| Example 330 | Int-E11 | J = 13.3 Hz, 1H), 2.10-2.04 (m, 2H), 1.95 |
| | | (dd, J = 24.8, 11.8 Hz, 1H). ¹⁹F NMR |
| | | (376 MHz, CD₃OD): δ −63.7 (s, 3F), |
| | | −74.6 (s, 3F). |

### Method 8: Synthesis of Example 88, 98, 100

### Example 88:5-[(2R,4S)-4-[6,7-dimethyl-4-[3-(trifluoromethyl)-1-bicyclo[1.1.1]pentanyl]pteridin-2-yl]tetrahydropyran-2-yl]-1-[1,2,2,2-tetradeuterio-1-(trideuteriomethyl)ethyl]pyridin-2-one

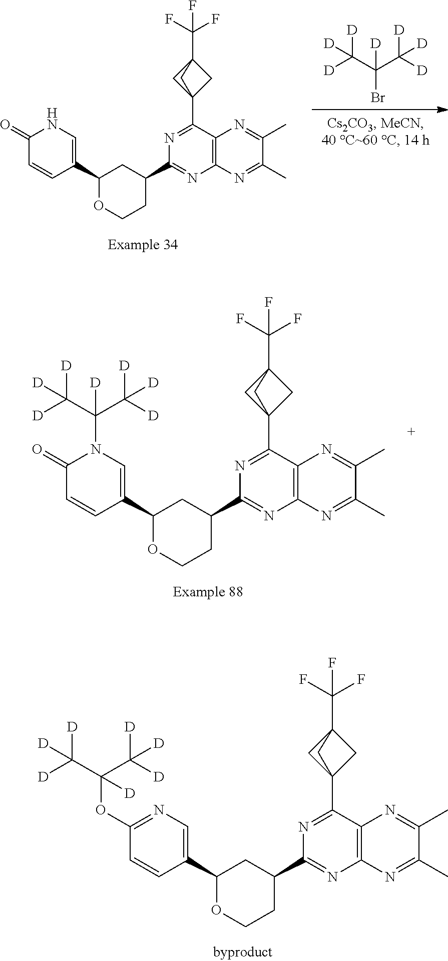

To a solution of 2-bromo-1,1,1,2,3,3,3-heptadeuterio-propane (1.5 equiv., 83 mg, 0.636 mmol) and 5-[(2R,4S)-4-[6,7-dimethyl-4-[3-(trifluoromethyl)-1-bicyclo[1.1.1]pentanyl]pteridin-2-yl]tetrahydropyran-2-yl]-1H-pyridin-2-one (Example 34, 1 equiv., 200 mg, 0.424 mmol) in MeCN (2 mL) was added Cs₂CO₃ (3 equiv., 415 mg, 1.27 mmol). The mixture was stirred at 40° C. for 12 hours. LCMS showed 50% raw material remained and two peaks (22% and 28%) with desired mass was detected. The solution was heated to 60° C. and stirred at 60° C. for another 2 hours. LCMS showed two peaks (33% and 54%) with desired mass (33%). The reaction mixture was filtered through a pad of Celite. The filter cake was washed with DCM (2×10 mL). The combined organic layers were concentrated under reduced pressure to give a residue. The residue was purified by reversed-phase chromatography (60-75% [water (FA)-MeCN]), concentrated, and the residual aqueous solution was lyophilized to give 5-[(2R,4S)-4-[6,7-dimethyl-4-[3-(trifluoromethyl)-1-bicyclo[1.1.1]pentanyl]pteridin-2-yl]tetrahydropyran-2-yl]-1-[1,2,2,2-tetradeuterio-1-(trideuteriomethyl)ethyl]pyridin-2-one (Example 88, 42 mg, 0.0814 mmol, 19% yield) as a solid and 6,7-dimethyl-2-[(2R,4S)-2-[6-[1,2,2,2-tetradeuterio-1-(trideuteriomethyl) ethoxy]-3-pyridyl]tetrahydropyran-4-yl]-4-[3-(trifluoromethyl)-1-bicyclo[1.1.1]pentanyl]pteridine (byproduct, 42 mg, 0.0797 mmol, 19% yield) as a solid.

Example 88: LCMS: [M+H]⁺=521.2; purity=100% (220 nm); Ret. time=0.538 min. 1H NMR (400 MHz, CDCl₃) δ 7.38 (br d, J=1.9 Hz, 1H), 7.35 (d, J=2.4 Hz, 1H), 6.56 (d, J=9.1 Hz, 1H), 4.40-4.23 (m, 2H), 3.87-3.72 (m, 1H), 3.54-3.38 (m, 1H), 2.79 (d, J=13.8 Hz, 6H), 2.65 (s, 6H), 2.30 (brd, J=13.4 Hz, 1H), 2.20-2.10 (m, 2H), 2.07-1.95 (m, 1H).

byproduct: LCMS: [M+H]⁺=521.2; purity=99% (220 nm); Ret. time=0.636 min. 1H NMR (400 MHz, CDCl₃) δ 8.15 (d, J=2.0 Hz, 1H), 7.66 (dd, J=2.4, 8.5 Hz, 1H), 6.67 (d, J=8.6 Hz, 1H), 4.56-4.45 (m, 1H), 4.31 (br dd, J=3.1, 10.4 Hz, 1H), 3.83 (dt, J=3.6, 11.3 Hz, 1H), 3.56-3.38 (m, 1H), 2.79 (d, J=14.3 Hz, 6H), 2.64 (s, 6H), 2.31 (br d, J=13.4 Hz, 1H), 2.24-2.13 (m, 2H), 2.12-2.03 (m, 1H).

An analogous method was followed to obtain the following compounds.

| Structure | SM | Characterization |
|---|---|---|
| 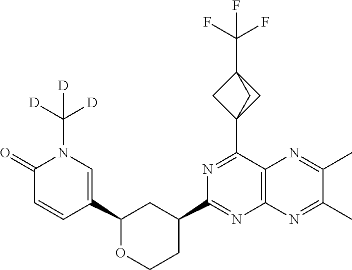 [SMILES: [2H]C([2H])([2H])n1cc([C@H]2C[C@@H](c3nc(C45CC(C(F)(F)F)(C4)C5)c4nc(C)c(C)nc4n3)CCO2)ccc1=O] | CD₃I | ESI-MS: [M + H]⁺ = 489.3. ¹H NMR (CDCl₃, 400 MHz): δ 7.38-7.41 (2H, m), 6.56 (1H, d, J = 9.1 Hz), 4.30 (2H, t, J = 12.6 Hz), 3.75-3.82 (1H, m), 3.41-3.49 (1H, m), 2.81 (3H, s), 2.77 (3H, s), 2.64 (6H, s), 2.31 (1H, d, J = 13.4 Hz), 1.98-2.15 (3H, m). ¹⁹F NMR (CDCl₃, 376 MHz): δ −73.1 (3F, s). |
| Example 36 | | |
| 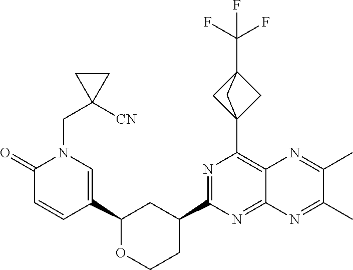 [SMILES: Cc1nc2nc([C@H]3CCO[C@@H](c4ccc(=O)n(CC5(C#N)CC5)c4)C3)nc(C34CC(C(F)(F)F)(C3)C4)c2nc1C] | 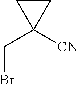 | Modifications: The reaction was ran at 40 °C for 2 hours. The residue was purified by Prep-TLC (100% EtOAc, desired product R_{f} = 0.35) to afford the 1-[[5- [(2R,4S)-4-[6,7-dimethyl-4-[3-(trifluoromethyl)-1- bicyclo[1.1.1]pentanyl] pteridin-2- yl]tetrahydropyran-2-yl]-2-oxo-1- pyridyl]methyl]cyclopropanecarbonitrile (Example 98, 33 mg, 0.057 mmol, 29% yield) as a solid, and 1- [[5-[(2R,4S)-4-[6, 7-dimethyl-4-[3-(trifluoromethyl)- 1-bicyclo-[1.1.1]pentanyl]pteridin-2- yl]tetrahydropyran-2-yl]-2-pyridyl]- oxymethyl]cyclopropane-carbonitrile (byproduct, 7.7 mg, 0.014 mmol, 7% yield) was obtained as a solid. Example 98: LMCS: [M + H]⁺ = 551.3; purity = 95% (220 nm); Ret. time = 0.581 min. ¹H NMR (400 MHz, CDCl₃) δ 7.54 (d, J = 2.1 Hz, 1H), 7.48 (dd, J = 2.4, |
| | | 9.4 Hz, 1H), 6.59 (d, J = 9.4 Hz, 1H), 4.37 (br d, J = |
| Example 98 | | 10.1 Hz, 1H), 4.33-4.25 (m, 1H), 4.08 (d, J = 1.3 Hz, |
| | | 2H), 3.80 (dt, J = 4.1, 11.1 Hz, 1H), 3.51-3.38 (m, |
| 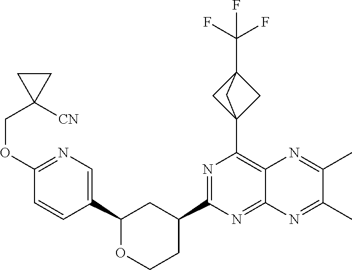 [SMILES: Cc1nc2nc([C@H]3CCO[C@@H](c4ccc(OCC5(C#N)CC5)nc4)C3)nc(C34CC(C(F)(F)F)(C3)C4)c2nc1C] | | 1H), 2.79 (d, J = 14.1 Hz, 6H), 2.65 (s, 6H), 2.34 (br d, J = 13.1 Hz, 1H), 2.19-2.08 (m, 2H), 2.07-1.98 (m, 1H), 1.46-1.36 (m, 2H), 1.35-1.28 (m, 2H). byproduct: LCMS: [M + H]⁺ = 551.3; purity = 96% (220 nm); Ret. time = 0.633 min. ¹H NMR (400 MHz, CDCl₃) δ 8.12 (d, J = 2.1 Hz, 1H), 7.71 (dd, J = 2.3, 8.6 Hz, 1H), 6.82 (d, J = 8.6 Hz, 1H), 4.60-4.46 (m, 1H), 4.35-4.29 (m, 3H), 3.90-3.76 (m, 1H), 3.59- 3.40 (m, 1H), 2.79 (d, J = 14.3 Hz, 6H), 2.65 (s, 6H), 2.32 (br d, J = 13.1 Hz, 1H), 2.22-2.12 (m, 2H), 2.11- 2.00 (m, 1H), 1.38-1.33 (m, 2H), 1.16-1.11 (m, 2H). |
| byproduct | | |
| 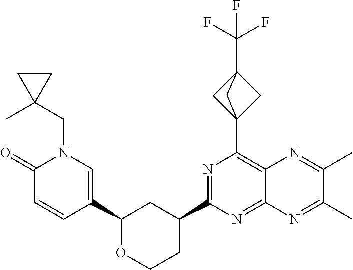 [SMILES: Cc1nc2nc([C@H]3CCO[C@@H](c4ccc(=O)n(CC5(C)CC5)c4)C3)nc(C34CC(C(F)(F)F)(C3)C4)c2nc1C] | 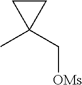 Int-A49 | Modifications: The reaction was ran at 60 °C for 12 hours. The crude was then purified by prep-HPLC (FA) and freeze-drying to give 5-[(2R,4S)-4-[6,7-dimethyl-4- [3-(trifluoromethyl)-1- bicyclo[1.1.1]pentanyl]pteridin-2-yl]tetrahydropyran- 2-yl]-1-[(1-methylcyclopropyl)methyl]pyridin-2-one (Example 100, 26 mg, 0.0467 mmol, 11% yield) as a solid and 6,7-dimethyl-2-[(2R,4S)-2-[6-[(1- methylcyclopropyl)methoxy]-3-pyridyl]- tetrahydropyran-4-yl]-4-[3-(trifluoro-methyl)-1- bicyclo[1.1.1]pentanyl]pteridine (byproduct, 5.2 mg, 0.01 mmol, 2% yield) as a solid. |
| Example 100 | | |
| 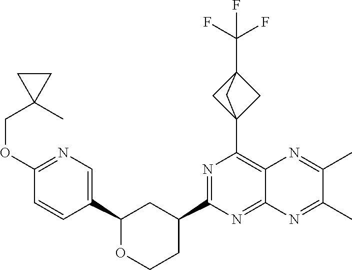 [SMILES: Cc1nc2nc([C@H]3CCO[C@@H](c4ccc(OCC5(C)CC5)nc4)C3)nc(C34CC(C(F)(F)F)(C3)C4)c2nc1C] | | Example 100: LCMS: [M + H]⁺ = 540.3, purity = 97% (220 nm). Ret. Time = 0.621 min. ¹H NMR (400 MHz, CDCl₃) δ 0.37-0.45 (m, 2H), 0.62-0.71 (m, 2H), 1.06 (s, 3H), 1.91-2.08 (m, 1H), 2.11-2.21 (m, 2H), 2.31 (br d, J = 13.1 Hz, 1H), 2.66 (s, 6H), 2.80 (d, J = 14.0 Hz, 6H), 3.41-3.53 (m, 1H), 3.74-3.85 (m, 1H), 3.89 (s, 2H), 4.19-4.47 (m, 2H), 6.53-6.63 (m, 1H), 7.36-7.44 (m, 2H). byproduct: LCMS: [M + H]⁺ = 540.2, purity = 100% byproduct CDCl₃) δ 0.36-0.43 (m, 2H), 0.51-0.60 (m, 2H), (220 nm). Ret. Time = 0.693 min. ¹H NMR (400 MHz, 1.22 (s, 3H), 2.04-2.23 (m, 3H), 2.26-2.37 (m, 1H), 2.65 (s, 6H), 2.79 (d, J = 14.5 Hz, 6H), 3.44-3.57 (m, 1H), 3.83 (td, J = 11.4, 3.8 Hz, 1H), 4.08 (s, 2H), 4.25- 4.40 (m, 1H), 4.52 (dd, J = 11.5, 2.0 Hz, 1H), 6.78 (d, J = 8.5 Hz, 1H), 7.68 (dd, J = 8.6, 2.4 Hz, 1H), 8.13 (d, |
| byproduct | | J = 2.4 Hz, 1H). |

### Method 9: Synthesis of Example 104-105, 109-110, 114, 116, and 153

### Example 104:5-((2R)-4-(6,7-dimethyl-4-(3-(trifluoromethyl) bicyclo-[1.1.1]pentan-1-yl) pteridin-2-yl)tetrahydro-2H-pyran-2-yl)-1-(5-methylthiophen-3-yl)pyridin-2 (1H)-one

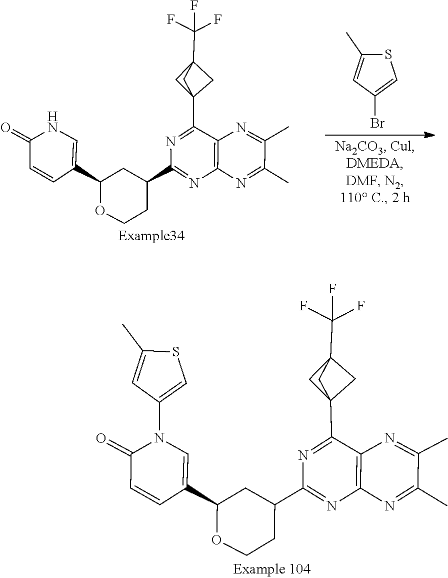

To a solution of 5-((2R,4S)-4-(6,7-dimethyl-4-(3-(trifluoromethyl) bicyclo[1.1.1]pentan-1-yl) pteridin-2-yl)tetrahydro-2H-pyran-2-yl)pyridin-2 (1H)-one (Example 34, 1 equiv., 150 mg, 0.318 mmol) in DMF (5 mL) was added 4-bromo-2-methylthiophene (1.5 equiv., 0.061 mL, 0.477 mmol), Na₂CO₃ (2 equiv., 88 mg, 0.636 mmol), DMEDA (0.4 equiv., 11 mg, 0.127 mmol), and CuI (0.2 equiv., 12 mg, 0.0636 mmol), and the mixture was stirred at 110° C. under N₂ atmosphere for 2 h. LCMS showed that the starting material was consumed completely and the desired mass was detected (63%). The reaction mixture was diluted with water (40 mL) and extracted with EtOAc (2×40 mL). The combined organic layers were washed with brine (50 mL), dried over Na₂SO₄, filtered, and concentrated under reduced pressure to give a residue. The residue was purified by prep-TLC (100% EtOAc,; desired product R_{f}=0.6) and lyophilized to give 5-((2R)-4-(6,7-dimethyl-4-(3-(trifluoromethyl) bicyclo-[1.1.1]pentan-1-yl) pteridin-2-yl)tetrahydro-2H-pyran-2-yl)-1-(5-methylthiophen-3-yl)pyridin-2 (1H)-one (Example 104, 86 mg, 0.142 mmol, 45% yield) as a solid. LCMS: [M+H]⁺=568.1; purity=94% (220 nm); Ret. time=0.634 min. ¹H NMR (400 MHz, CDCl₃) δ 7.49-7.41 (m, 2H), 7.15 (d, J=1.5 Hz, 1H), 6.96-6.89 (m, 1H), 6.67-6.60 (m, 1H), 4.39-4.24 (m, 2H), 3.80 (dt, J=3.4, 11.5 Hz, 1H), 3.53-3.39 (m, 1H), 2.81 (s, 3H), 2.78 (s, 3H), 2.65 (s, 6H), 2.51 (d, J=0.9 Hz, 3H), 2.33 (br dd, J=1.8, 13.1 Hz, 1H), 2.19-1.99 (m, 3H). 83% ee.

### Example 105:5-((2R,4S)-4-(6,7-dimethyl-4-(3-(trifluoromethyl) bicyclo[1.1.1]pentan-1-yl) pteridin-2-yl)tetrahydro-2H-pyran-2-yl)-1-(5-methylthiophen-3-yl)pyridin-2 (1H)-one

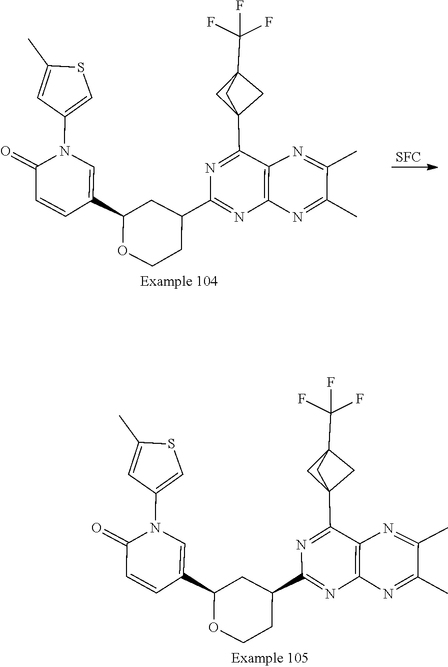

5-((2R)-4-(6,7-dimethyl-4-(3-(trifluoromethyl) bicyclo[1.1.1]pentan-1-yl) pteridin-2-yl)tetrahydro-2H-pyran-2-yl)-1-(5-methylthiophen-3-yl)pyridin-2 (1H)-one (Example 104, 1 equiv., 83 mg, 0.146 mmol) was purified by SFC (Condition 3, Gradient a) to give 60 mg the desired product, LCMS showed 95% purity. The product was lyophilized to give 50 mg the desired product, LCMS showed 80% purity. 50 mg of the desired product was purified by reversed-phase chromatography (40-60% [water (FA)-ACN], in 10 min) and lyophilized to give 5-((2R,4S)-4-(6,7-dimethyl-4-(3-(trifluoromethyl) bicyclo[1.1.1]pentan-1-yl) pteridin-2-yl)tetrahydro-2H-pyran-2-yl)-1-(5-methylthiophen-3-yl)pyridin-2 (1H)-one (Example 105, 32 mg, 0.052 mmol) as a solid. LCMS: [M+H]⁺=568.1; purity=93% (220 nm); Ret. time=0.636 min; 97% ee. ¹H NMR (400 MHz, CDCl₃) δ 7.50-7.40 (m, 2H), 7.14 (d, J=1.4 Hz, 1H), 6.92 (s, 1H), 6.71-6.54 (m, 1H), 4.40-4.25 (m, 2H), 3.79 (dt, J=3.4, 11.4 Hz, 1H), 3.53-3.39 (m, 1H), 2.79 (d, J=13.6 Hz, 6H), 2.65 (s, 6H), 2.50 (s, 3H), 2.33 (br d, J=13.1 Hz, 1H), 2.18-1.98 (m, 3H).

An analogous method was followed to obtain the following compounds.

| | | Starting | |
|---|---|---|---|
| Ex # | Structure | Material | Characterization |
| 109, 110 | 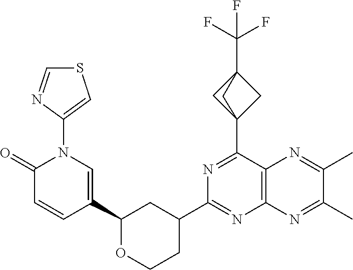 [SMILES: Cc1nc2nc(C3CCO[C@@H](c4ccc(=O)n(-c5cscn5)c4)C3)nc(C34CC(C(F)(F)F)(C3)C4)c2nc1C] | 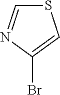 | The crude residue was purified by silica gel column chromatography (0-80% EtOAc in PE, 30 mL/min) and lyophilized to give 5-((2R)-4-(6,7-dimethyl-4-(3- (trifluoromethyl)bicyclo-[1.1.1]pentan-1- yl)pteridin-2-yl)tetrahydro-2H-pyran-2- yl)-1-(thiazol-4-yl)pyridin-2(1H)-one (Example 109, 28 mg, 0.049 mmol, 23% yield) as a solid. LCMS (mixture of diastereomers): [M + H]⁺ = 555.2; purity = 97% (220 nm); Ret. time = 0.377 min; 89% ee. ¹H NMR (400 MHz, CDCl₃) δ 8.75 (d, J = 2.4 Hz, 1H), 8.51 (d, J = 2.4 Hz, 1H), 8.37 (d, J = 2.4 Hz, 1H), 7.50 (dd, J = 2.5, 9.4 Hz, 1H), 6.70 (d, J = 9.5 Hz, 1H), 4.46-4.38 (m, 1H), 4.35-4.28 (m, 1H), 3.88-3.74 (m, 1H), 3.48 (br dd, J = |
| | | | 3.2, 6.9 Hz, 1H), 2.82 (s, 3H), 2.78 (s, 3H), |
| | Example 109 | | 2.66 (s, 6H), 2.36 (br d, J = 13.3 Hz, 1H), |
| | Then, SFC- | | 2.20-2.05 (m, 3H). |
| | | | Then, SFC- |
| | 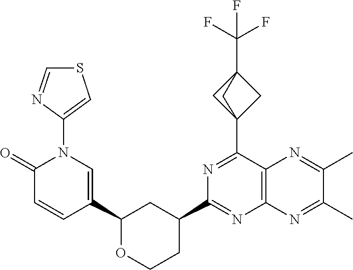 [SMILES: Cc1nc2nc([C@H]3CCO[C@@H](c4ccc(=O)n(-c5cscn5)c4)C3)nc(C34CC(C(F)(F)F)(C3)C4)c2nc1C] | | 5-((2R)-4-(6,7-dimethyl-4-(3- (trifluoromethyl) bicyclo[1.1.1]pentan-1- yl)pteridin-2-yl)tetrahydro-2H-pyran-2- yl)-1-(thiazol-4-yl)pyridin-2(1H)-one (Example 109, 1 equiv., 20 mg, 0.036 mmol) was purified by SFC (Condition 3, Gradient b) and lyophilized to give 5- ((2R,4S)-4-(6,7-dimethyl-4-(3- (trifluoromethyl)bicyclo[1.1.1]pentan-1- yl)pteridin-2-yl)tetrahydro-2H-pyran-2- yl)-1-(thiazol-4-yl)pyridin-2(1H)-one (Example 110, 10 mg, 0.018 mmol) as a solid. LCMS: [M + H]⁺ = 555.2; purity = 96% (220 nm); Ret. time = 0.601 min; 100% ee. ¹H NMR (400 MHz, CDCl₃) δ 8.75 (d, J = 2.3 Hz, 1H), 8.50 (d, J = 2.1 Hz, 1H), 8.36 (d, J = 2.3 Hz, 1H), 7.50 (dd, |
| | | | J = 2.4, 9.4 Hz, 1H), 6.70 (d, J = 9.5 Hz, |
| | Example 110 | | 1H), 4.42 (br d, J = 11.4 Hz, 1H), 4.31 (br |
| | | | dd, J = 3.0, 11.0 Hz, 1H), 3.86-3.75 (m, |
| | | | 1H), 3.59-3.41 (m, 1H), 2.80 (d, J = 14.3 |
| | | | Hz, 6H), 2.65 (s, 6H), 2.36 (br d, J = 13.5 |
| | | | Hz, 1H), 2.18-2.02 (m, 3H). |
| 114 | 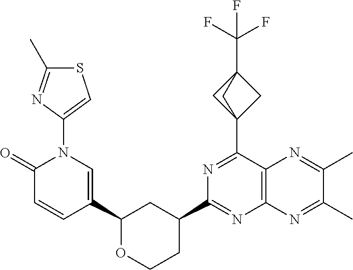 [SMILES: Cc1nc(-n2cc([C@H]3C[C@@H](c4nc(C56CC(C(F)(F)F)(C5)C6)c5nc(C)c(C)nc5n4)CCO3)ccc2=O)cs1] | 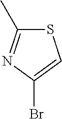 | Modification: The reaction was ran at 100 °C for 1.5 hours. The residue was purified by reversed- phase chromatography (45-75% [water (0.1% FA)-ACN], in 12 min) and lyophilized to give 5-((2R,4S)-4-(6,7- dimethyl-4-(3-(trifluoro- methyl)bicyclo[1.1.1]pentan-1- yl)pteridin-2-yl)tetrahydro-2H-pyran-2- yl)-1-(2-methyl-thiazol-4-yl)pyridin- 2(1H)-one (Example 114, 2.5 mg, 0.004 mmol, 5% yield) as a solid. LCMS: [M + H]⁺ = 569.3; purity = 98% (220 nm); Ret. time = 0.622 min. 92% ee. ¹H NMR (400 MHz, CDCl₃) δ 8.41 (d, J = 2.5 Hz, 1H), 8.05 (s, 1H), 7.48 (dd, J = 2.5, 9.5 Hz, 1H), 6.67 (d, J = 9.4 Hz, 1H), 4.42 (dd, |
| | | | J = 1.6, 11.3 Hz, 1H), 4.35-4.26 (m, 1H), |
| | Example 114 | | 3.86-3.75 (m, 1H), 3.53-3.42 (m, 1H), |
| | | | 2.82 (s, 3H), 2.78 (s, 3H), 2.74 (s, 3H), |
| | | | 2.66 (s, 6H), 2.34 (br d, J = 13.5 Hz, 1H), |
| | | | 2.19-2.05 (m, 3H). |
| 116 | 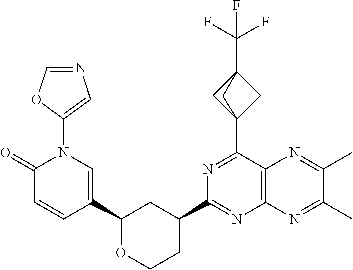 [SMILES: Cc1nc2nc([C@H]3CCO[C@@H](c4ccc(=O)n(-c5cnco5)c4)C3)nc(C34CC(C(F)(F)F)(C3)C4)c2nc1C] | 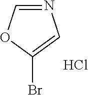 | Modification: The reaction was ran at 100 °C The crude product was purified by prep- HPLC (Condition 5, Gradient g) and combined with another batch. The combined phase was lyophilized to give 5- ((2R,4S)-4-(6,7-dimethyl-4-(3- (trifluoromethyl)-bicyclo-[1.1.1]pentan- 1-yl)pteridin-2-yl)tetrahydro-2H-pyran- 2-yl)-1-(oxazol-5-yl)pyridin-2(1H)-one (Example 116, 1.6 mg, 0.003 mmol, 3% yield) as a solid. LCMS: [M + H]⁺ = 539.2; purity = 97% (220 nm); Ret. time = 0.571 min; 91.7% ee. ¹H NMR (400 MHz, CDCl₃) δ 7.82 (s, 1H), 7.77 (s, 1H), 7.63 (s, 1H), 7.46 (dd, J = 2.3, 9.4 Hz, 1H), 6.67 (d, J = 9.5 Hz, 1H), 4.40 (br d, J = 10.9 Hz, |
| | | | 1H), 4.36-4.27 (m, 1H), 3.82 (dt, J = 2.8, |
| | Example 116 | | 11.6 Hz, 1H), 3.55-3.38 (m, 1H), 2.80 (d, |
| | | | J = 14.0 Hz, 6H), 2.65 (s, 6H), 2.37 (br d, |
| | | | J = 13.9 Hz, 1H), 2.21-2.08 (m, 2H), 2.07- |
| | | | 2.01 (m, 1H). |

An analogous method was followed to obtain the following compounds.

| Ex | | | |
|---|---|---|---|
| # | Structure | Starting Material | Characterization |
| 153 | 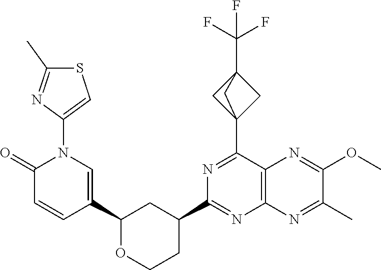 [SMILES: COc1nc2c(C34CC(C(F)(F)F)(C3)C4)nc([C@H]3CCO[C@@H](c4ccc(=O)n(-c5csc(C)n5)c4)C3)nc2nc1C] | 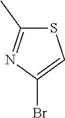 | Modification: The reaction was ran at 80 ºC for 2.5 hours. The residue was purified by reversed-phase chromatography (45- 75% [water (0.1% FA)- ACN], in 12 min) and lyophilized to give 5- ((2R,4S)-4-(6-methoxy- 7-methyl-4-(3- (trifluoromethyl)- bicyclo[1.1.1]pentan-1- yl)pteridin-2- yl)tetrahydro-2H-pyran- 2-yl)-1-(2- methylthiazol-4- |
| | | | yl)pyridin-2(1H)-one |
| | Example 153 | 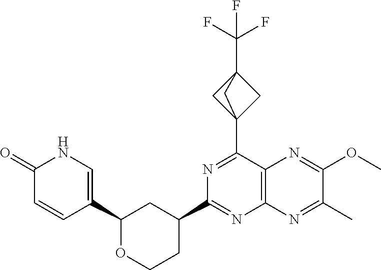 | (Example 153, 1.9 mg, 0.003 mmol, 4% yield) as a solid. LCMS: [M + H]⁺ = 585.3; purity = 96% (220 nm); Ret. time = 0.627 min. 92% ee. ¹H NMR (400 MHz, CDCl₃) δ 8.41 (d, J = 2.4 Hz, 1H), 8.05 (s, 1H), 7.57-7.41 (m, 1H), 6.67 (d, J = 9.5 Hz, 1H), 4.41 (dd, J = 1.6, 11.3 Hz, 1H), 4.35-4.26 (m, 1H), 4.15 (s, 3H), 3.86-3.75 (m, 1H), 3.45 (ddd, J = 3.8, 11.8, 15.6 Hz, 1H), 2.74 (d, J = 3.0 Hz, 6H), |
| | | | 2.64 (s, 6H), 2.32 (br d, |
| | | Example 143 | J = 13.4 Hz, 1H), 2.19- |
| | | | 2.05 (m, 3H). |

### Method 10: Synthesis of Examples 120 and 121

### Example 120:1-(1-bicyclo[1.1.1]pentanyl)-5-[(2R,4S)-4-[6,7-dimethyl-4-[3-(trifluoromethyl)-1-bicyclo-[1.1.1]pentanyl]pteridin-2-yl]tetrahydropyran-2-yl]pyridin-2-one; Example 121:1-(1-bicyclo[1.1.1]pentanyl)-5-[(2S,4R)-4-[6,7-dimethyl-4-[3-(trifluoromethyl)-1-bicyclo[1.1.1]-pentanyl]pteridin-2-yl]tetrahydropyran-2-yl]pyridine-2-one

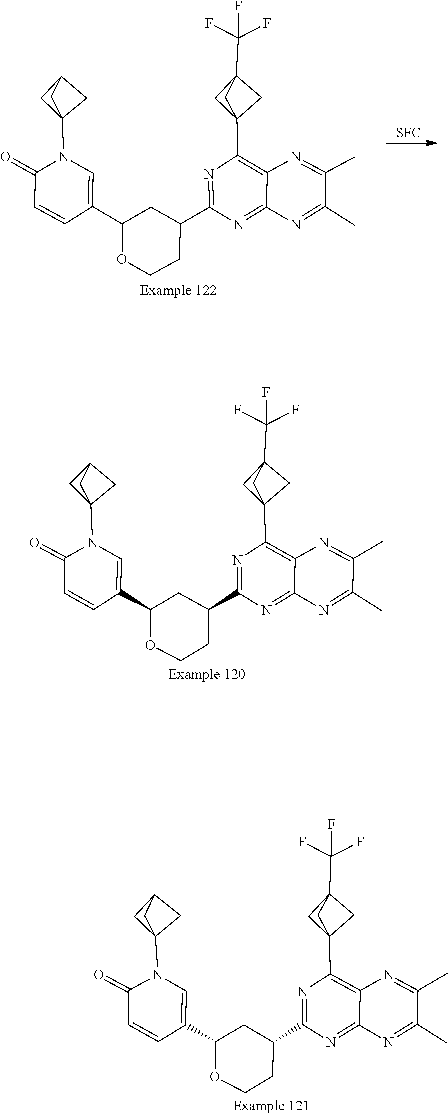

The residue (500 mg) was purified by silica gel column chromatography (0-100% EtOAc in Petroleum ether & EtOAc in MeOH)(TLC, 25% MeOH in EtOAc, desired product R_{f}=0.3) to give 1-(1-bicyclo[1.1.1]pentanyl)-5-[4-[6,7-dimethyl-4-[3-(trifluoromethyl)-1-bicyclo[1.1.1]-pentanyl]pteridin-2-yl]tetrahydropyran-2-yl]pyridin-2-one (Example 122, 120 mg, 0.22 mmol, 85% purity) as a solid. It was then purified by silica gel column chromatography (0-100% EtOAc in PE, then 0-50% MeOH in EtOAc) (TLC, 9% MeOH in DCM, R_{f}=0.3) to give the residue (~98% purity, 35 mg). The residue was purified by SFC (Condition 5, Gradient a-2) to give the Crude A (~10 mg, ~85% purity) and the Crude B (~20 mg, ~50% purity).

The Crude A was purified again by Prep-HPLC (Condition 5) and lyophilized to give 1-(1-bicyclo[1.1.1]pentanyl)-5-[(2R,4S)-4-[6,7-dimethyl-4-[3-(trifluoromethyl)-1-bicyclo-[1.1.1]pentanyl]pteridin-2-yl]tetrahydropyran-2-yl]pyridin-2-one (Example 120, 2.0 mg, 0.003 mmol, 1.5% yield) as a solid.

The Crude B was purified by SFC (Condition 4, Gradient a), concentrated under reduced pressure, and further purified by Prep-HPLC (Condition 5) and lyophilized to give 1-(1-bicyclo[1.1.1]pentanyl)-5-[(2S,4R)-4-[6,7-dimethyl-4-[3-(trifluoromethyl)-1-bicyclo[1.1.1]-pentanyl]pteridin-2-yl]tetrahydropyran-2-yl]pyridine-2-one (Example 121, 1.3 mg, 0.0024 mmol, 1% yield) as a solid.

Example 120: LCMS: [M+H]⁺=538.3; purity=100% (220 nm); Ret. time=0.625 min. ¹H NMR (400 MHz, CDCl₃) δ 7.36 (dd, J=2.5, 9.4 Hz, 1H), 7.26 (d, J=2.4 Hz, 1H), 6.47 (d, J=9.4 Hz, 1H), 4.36-4.24 (m, 2H), 3.85-3.74 (m, 1H), 3.39 (s, 1H), 2.80 (d, J=14.1 Hz, 6H), 2.66-2.63 (m, 7H), 2.38 (s, 6H), 2.28 (br d, J=13.1 Hz, 1H), 2.19-2.10 (m, 2H), 2.07-1.97 (m, 2H). ee ~92%.

Example 121: LCMS: [M+H]⁺=538.3; purity=100% (220 nm); Ret. time=0.626 min. ¹H NMR (400 MHz, CDCl₃) δ 7.36 (dd, J=2.5, 9.4 Hz, 1H), 7.26 (br s, 1H), 6.47 (d, J=9.4 Hz, 1H), 4.35-4.26 (m, 2H), 3.85-3.73 (m, 1H), 3.52-3.40 (m, 1H), 2.80 (d, J=14.1 Hz, 6H), 2.66-2.64 (m, 6H), 2.39 (s, 6H), 2.31-2.24 (m, 1H), 2.17-2.11 (m, 2H), 2.08-1.97 (m, 1H). ee: 92.5%.

### Method 11: Synthesis of Examples 126-128

### Example 128:5-[4-[6-(difluoromethyl)-4-[2-fluoro-4-(trifluoromethyl)phenyl]-7-methyl-pteridin-2-yl]tetrahydropyran-2-yl]-1-methyl-pyridin-2-one

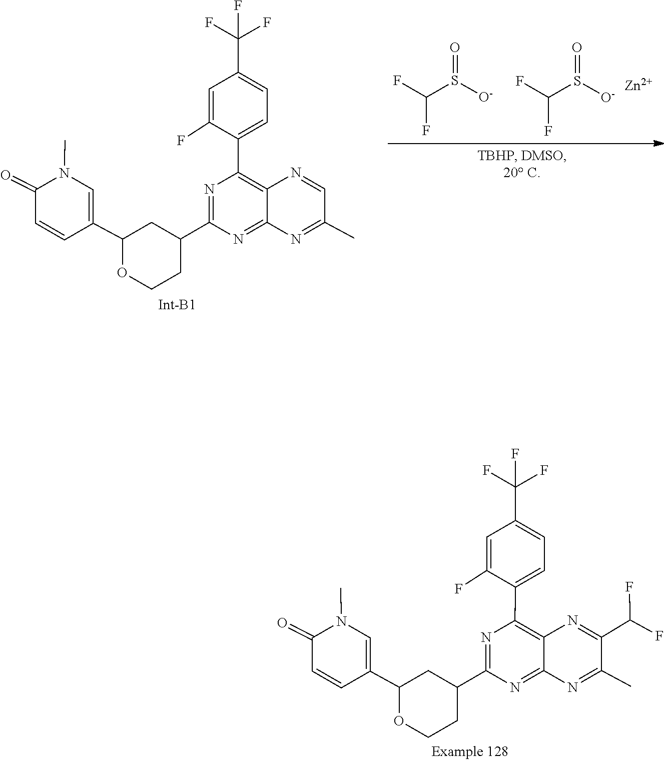 [SMILES: Cc1cnc2c(-c3ccc(C(F)(F)F)cc3F)nc(C3CCOC(c4ccc(=O)n(C)c4)C3)nc2n1 ; O=S([O-])C(F)F ; O=S([O-])C(F)F ; [Zn+2] ; Cc1nc2nc(C3CCOC(c4ccc(=O)n(C)c4)C3)nc(-c3ccc(C(F)(F)F)cc3F)c2nc1C(F)F]

To a solution of 5-[4-[4-[2-fluoro-4-(trifluoromethyl)phenyl]-7-methyl-pteridin-2-yl]tetrahydropyran-2-yl]-1-methyl-pyridin-2-one (Int-B1, 1 equiv., 100 mg, 0.2 mmol) and zinc difluoromethanesulfinate (2.7 equiv., 159 mg, 0.54 mmol) in DMSO (5 mL) at 25° C., tert-butylhydroperoxide (5 equiv., 129 mg, 1 mmol) was added with vigorous stirring and bubbled with N₂ for 30 seconds, and the reaction was stirred at 20° C. for 2 hours. Then, zinc difluoromethanesulfinate (2.7 equiv., 159 mg, 0.54 mmol) was added to the reaction mixture and then the mixture was stirred at 20° C. for another 12 hours. LCMS showed ~19% of starting material was remained and ~21% of desired product was detected. The mixture (combined with 3 additional batches) was quenched by 200 mL H₂O, extracted with DCM (3×150 mL), the combined organic layers was dried over anhydrous Na₂SO₄, filtered, and concentrated under reduced pressure to give a crude residue. The residue was purified by silica gel column chromatography (0-100% EtOAc in PE, then 0-50% MeOH in EtOAc)(TLC, 9% MeOH in DCM, desired product R_{f}=0.6) to give the crude product. The crude product was purified again by Prep-TLC (9% MeOH in DCM, desired product R_{f}=0.6) to give the 5-[4-[6-(difluoromethyl)-4-[2-fluoro-4-(trifluoromethyl)phenyl]-7-methyl-pteridin-2-yl]tetrahydropyran-2-yl]-1-methyl-pyridin-2-one (Example 128, 30 mg, 0.055 mmol, 27% yield) as a solid. LCMS: [M+H]⁺=500.1; purity=94% (UV 220 nm); Ret. time=0.565 min.

### Isolation of Example 126:5-[(2R,4S)-4-[6-(difluoromethyl)-4-[2-fluoro-4-(trifluoromethyl)phenyl]-7-methyl-pteridin-2-yl]tetrahydropyran-2-yl]-1-methyl-pyridin-2-one, Example 127:5-[(2S,4R)-4-[6-(difluoromethyl)-4-[2-fluoro-4-(trifluoromethyl)phenyl]-7-methyl-pteridin-2-yl]tetrahydropyran-2-yl]-1-methyl-pyridin-2-one

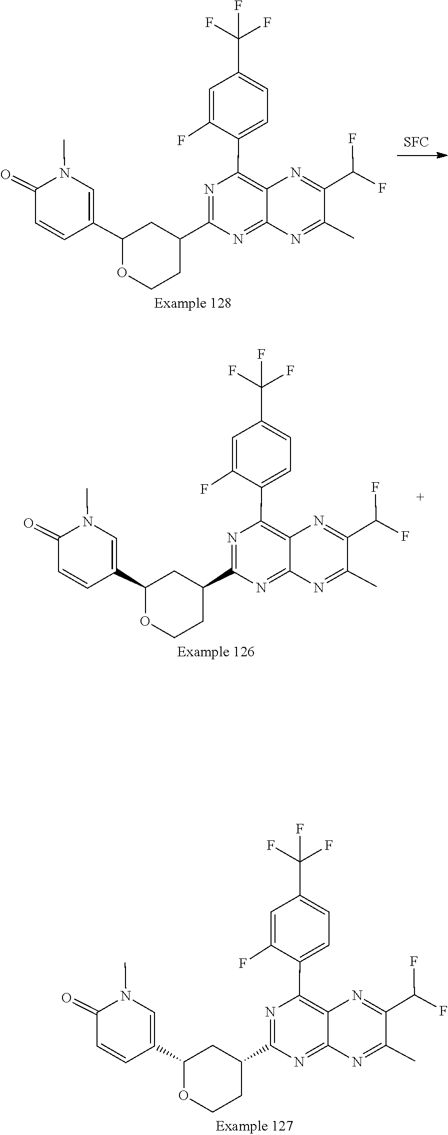

The crude (Example 128) was separated by SFC (Condition 14, Gradient a) to afford 5-[(2R,4S)-4-[6-(difluoromethyl)-4-[2-fluoro-4-(trifluoromethyl)phenyl]-7-methyl-pteridin-2-yl]tetrahydropyran-2-yl]-1-methyl-pyridin-2-one (Example 126, 6.3 mg, 0.011 mmol, 13% yield) as a solid, and 5-[(2S,4R)-4-[6-(difluoromethyl)-4-[2-fluoro-4-(trifluoromethyl)phenyl]-7-methyl-pteridin-2-yl]tetrahydropyran-2-yl]-1-methyl-pyridin-2-one (Example 127, 7.6 mg, 0.0138 mmol, 17% yield) as a solid.

Example 126: LCMS: [M+H]⁺=550.3; purity=94% (UV 220 nm); Ret. time=0.557 min. ¹H NMR (400 MHz, CDCl₃) δ 7.93-7.83 (m, 1H), 7.72-7.62 (m, 1H), 7.60-7.53 (m, 1H), 7.44-7.37 (m, 2H), 6.89-6.55 (m, 2H), 4.40-4.28 (m, 2H), 3.82 (dt, J=2.8, 11.8 Hz, 1H), 3.68-3.59 (m, 1H), 3.56 (s, 3H), 3.14-3.03 (m, 3H), 2.47-2.36 (m, 1H), 2.26-2.02 (m, 3H).

Example 127: LCMS: [M+H]⁺=550.3; purity=100% (UV 220 nm); Ret. time=0.557 min. ¹H NMR (400 MHz, CDCl₃) δ 7.93-7.83 (m, 1H), 7.87 (t, J=7.3 Hz, 1H), 7.66 (br d, J=7.9 Hz, 1H), 7.56 (br d, J=9.3 Hz, 1H), 7.45-7.35 (m, 2H), 6.88 (s, 1H), 6.74 (s, 1H), 6.68-6.46 (m, 1H), 4.40-4.30 (m, 2H), 3.82 (dt, J=2.8, 11.7 Hz, 1H), 3.68-3.60 (m, 1H), 3.56 (s, 3H), 3.08 (s, 3H), 2.43-2.38 (m, 1H), 2.24-2.13 (m, 3H).

### Method 12: Synthesis of Examples 129-130

### Example 129:1-methyl-5-[(2R,4S)-4-[4-[2-fluoro-4-(trifluoromethyl)phenyl]-6-methoxy-7-methyl-pteridin-2-yl]tetrahydropyran-2-yl]pyridin-2-one

### Example 130:1-methyl-5-[(2S,4R)-4-[4-[2-fluoro-4-(trifluoromethyl)phenyl]-6-methoxy-7-methyl-pteridin-2-yl]tetrahydropyran-2-yl]pyridin-2-one

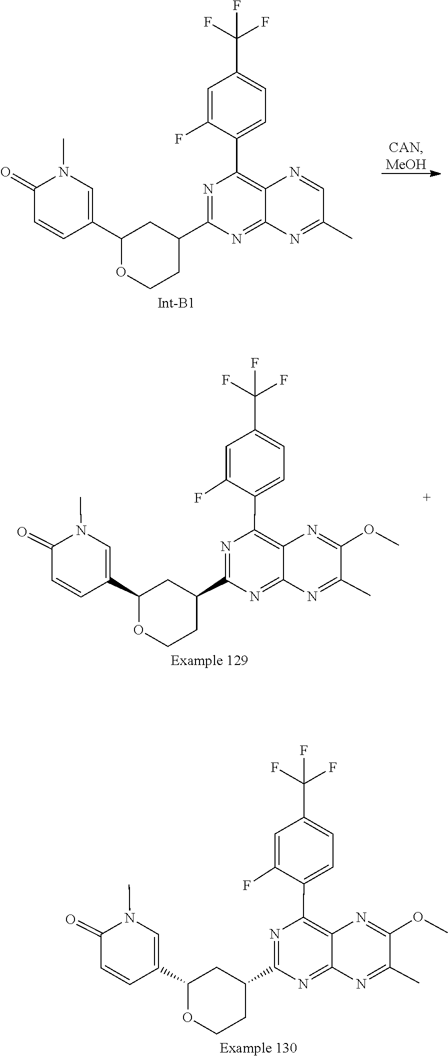

To a solution of 5-[4-[4-[2-fluoro-4-(trifluoromethyl)phenyl]-7-methyl-pteridin-2-yl]tetrahydropyran-2-yl]-1-methyl-pyridin-2-one (Int-B1, 1 equiv., 300 mg, 0.601 mmol) in MeOH (10 mL) was added CAN (1.5 equiv., 494 mg, 0.901 mmol) by portions at 0° C., and stirred for 20 min at 0° C. LCMS showed raw material was mostly consumed and the major peak showed desired mass. The reaction was poured into Na₂SO₃ (aq. 20 mL) and extracted with EtOAc (15 mL×3). The organics were washed with 20 mL saturated brine solution. The combined organic layers were dried over anhydrous Na₂SO₄, filtered, and concentrated under reduced pressure to give a crude residue. The crude was purified by Prep-NPLC (Condition 1) to give 100 mg desired product. Then 100 mg product was combined with 47 mg from another batch and separated by SFC (Condition 15, Gradient a) to give 30 mg Crude A product and 1-methyl-5-[(2S,4R)-4-[4-[2-fluoro-4-(trifluoromethyl)phenyl]-6-methoxy-7-methyl-pteridin-2-yl]tetrahydropyran-2-yl]pyridin-2-one (Example 130, 32 mg, 0.0600 mmol, 10% yield) as a solid. LCMS: Ret. time=1.391 min. MS [M+H]⁺=530.3, purity=100%, uv=220 nm, Ret. time=0.567 min. ¹H NMR (400 MHz, CDCl₃) δ 2.05-2.23 (m, 3H) 2.37 (br d, J=13.1 Hz, 1H), 2.78 (s, 3H), 3.52-3.57 (m, 4H), 3.77-3.85 (m, 1H) 4.01 (s, 3H) 4.21-4.45 (m, 2H) 6.51-6.64 (m, 1H), 7.36-7.44 (m, 2H) 7.52 (br d, J=9.8 Hz, 1H) 7.63 (d, J=8.1 Hz, 1H), 7.87 (t, J=7.2 Hz, 1H).

Crude A (30 mg) was purified by prep-TLC (9% MeOH in EtOAc, R_{f}=0.5) to give about 20 mg of oil, which was separated by SFC (Condition 1, Gradient a-1) to give 1-methyl-5-[(2R,4S)-4-[4-[2-fluoro-4-(trifluoromethyl)phenyl]-6-methoxy-7-methyl-pteridin-2-yl]tetrahydropyran-2-yl]pyridin-2-one (Example 129, 9.0 mg, 0.017 mmol, 3% yield) as a solid. LCMS: [M+H]⁺=530.2, purity=100% (220 nm), Ret. time=0.568 min. ¹H NMR (400 MHz, CDCl₃) δ 2.04-2.24 (m, 2H), 2.23-2.24 (m, 1H), 2.37 (br d, J=13.4 Hz, 1H), 2.77 (s, 3H), 3.49-3.58 (m, 4H), 3.75-3.86 (m, 1H), 4.01 (s, 3H), 4.24-4.40 (m, 2H), 6.52-6.62 (m, 1H), 7.37-7.43 (m, 2H), 7.52 (d, J=9.6 Hz, 1H), 7.62 (d, J=7.8 Hz, 1H), 7.86 (t, J=7.3 Hz, 1H).

### Method 13: Synthesis of Example 140, 145, 147, and 149

### Example 140:5-[(2R,4S)-4-[6-methoxy-7-methyl-4-[3-(trifluoromethyl)-1-bicyclo[1.1.1]pentanyl]pteridin-2-yl]tetrahydropyran-2-yl]-1-methyl-pyridin-2-one

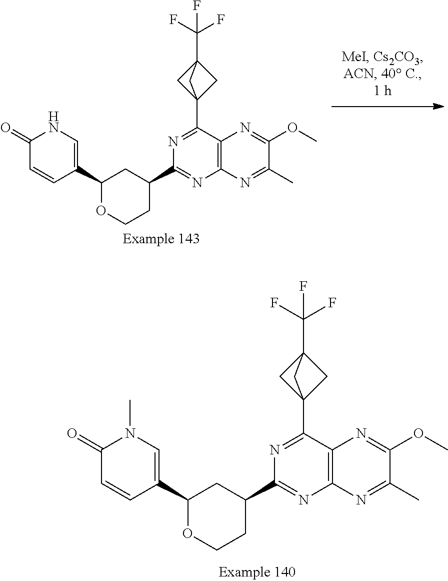 [SMILES: COc1nc2c(C34CC(C(F)(F)F)(C3)C4)nc([C@H]3CCO[C@@H](c4ccc(=O)[nH]c4)C3)nc2nc1C ; "MeI, Cs2CO3,ACN, 40° C., 1 h" ; COc1nc2c(C34CC(C(F)(F)F)(C3)C4)nc([C@H]3CCO[C@@H](c4ccc(=O)n(C)c4)C3)nc2nc1C]

To a mixture of 5-[(2R,4S)-4-[6-methoxy-7-methyl-4-[3-(trifluoromethyl)-1-bicyclo[1.1.1]pentanyl]pteridin-2-yl]tetrahydropyran-2-yl]-1H-pyridin-2-one (Example 143, 1 equiv., 40 mg, 0.0821 mmol) and Cs₂CO₃ (2 equiv., 53 mg, 0.164 mmol) in MeCN (2 mL) was added MeI (5 equiv., 0.026 mL, 0.410 mmol), and the reaction was stirred at 40° C. for 0.5 h. LCMS showed a major peak with desired mass. The mixture was combined with another batch, and the final mixture was added to 20 mL water and extracted with EtOAc (3×20 mL). The organic phase was concentrated under reduced pressure to give a crude residue which was purified by reversed phase-HPLC (100-0% [(0.05% FA) water:ACN]) twice and lyophilized to give 5-[(2R,4S)-4-[6-methoxy-7-methyl-4-[3-(trifluoromethyl)-1-bicyclo[1.1.1]pentanyl]pteridin-2-yl]tetrahydropyran-2-yl]-1-methyl-pyridin-2-one (Example 140, 24 mg, 0.0452 mmol, 55% yield) as a solid. LCMS: [M+H]⁺=502.2; purity=93% (UV 220 nm); Ret. time=0.577 min. ¹H NMR (400 MHz, CDCl₃) δ 7.49-7.36 (m, 2H), 6.68-6.53 (m, 1H), 4.43-4.25 (m, 2H), 4.16 (s, 3H), 3.80 (br d, J=3.6 Hz, 1H), 3.57 (s, 3H), 3.50-3.37 (m, 1H), 2.75 (s, 3H), 2.65 (s, 6H), 2.30 (br s, 1H), 2.20-2.08 (m, 2H), 2.08-1.97 (m, 1H). SFC: 95% purity.

An analogous method was followed to obtain the following compounds.

| | Starting | |
|---|---|---|
| Structure | Material | Characterization |
| 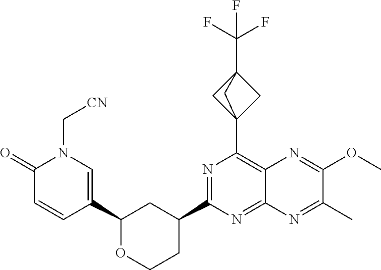 [SMILES: COc1nc2c(C34CC(C(F)(F)F)(C3)C4)nc([C@H]3CCO[C@@H](c4ccc(=O)n(CC#N)c4)C3)nc2nc1C] | 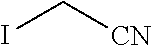 | Modification: The reaction was ran at 25 °C for 1 hour. The crude was purified by reversed phase-HPLC (100-0% [(0.05% FA) water:ACN]) and lyophilized to give 2-[5-[(2R,4S)-4-[6-methoxy- 7-methyl-4-[3-(trifluoromethyl)-1-bicyclo- [1.1.1]pentanyl]pteridin-2-yl]tetrahydropyran- 2-yl]-2-oxo-1-pyridyl]acetonitrile (Example 145, 4.3 mg, 0.008 mmol, 37% yield) as a solid. LCMS: [M + H]⁺ = 527.2; purity = 94% (UV 220 nm); Ret. time = 0.568 min. ¹H NMR (400 MHz, CDCl₃) δ 7.45 (qd, J = 2.5, 5.1 Hz, 2H), 6.68- 6.57 (m, 1H), 4.84 (s, 2H), 4.41-4.24 (m, 2H), 4.14 (s, 3H), 3.78 (dt, J = 3.0, 11.6 Hz, 1H), 3.48- 3.35 (m, 1H), 2.73 (s, 3H), 2.63 (s, 6H), 2.38- 2.29 (m, 1H), 2.19-2.06 (m, 2H), 2.02-1.91 (m, 1H). SFC: 88% purity. |
| Example 145 | | |
| 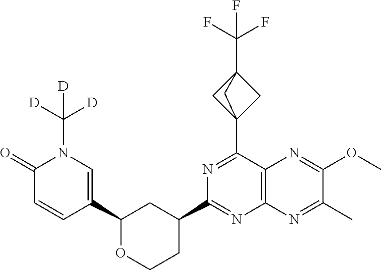 [SMILES: [2H]C([2H])([2H])n1cc([C@H]2C[C@@H](c3nc(C45CC(C(F)(F)F)(C4)C5)c4nc(OC)c(C)nc4n3)CCO2)ccc1=O] | CD₃I | Modification: The reaction was ran for 12 hours at 25 °C The crude product was purified by reversed- phase chromatography (30-70% [water (FA)- ACN], in 10 min) and lyophilized to give 5- ((2R,4S)-4-(6-methoxy-7-methyl-4-(3- (trifluoromethyl)-bicyclo[1.1.1]pentan-1- yl)pteridin-2-yl)tetrahydro-2H-pyran-2-yl)-1- (methyl-d3)pyridin-2(1H)-one (Example 147, 40 mg, 0.078 mmol, 38% yield) as a solid. LCMS: [M + H]+ = 505.3; purity = 99%; Ret. time = 0.580 min; 100% ee. ¹H NMR (400 MHz, CDCl₃) δ 7.46-7.36 (m, 2H), 6.57 (dd, J = 0.8, 8.9 Hz, 1H), 4.36-4.24 (m, 2H), 4.15 (s, 3H), 3.79 (dt, J = 3.6, 11.4 Hz, 1H), 3.50-3.36 (m, 1H), 2.74 (s, 3H), 2.64 (s, 6H), 2.30 (br dd, J = 1.9, 13.4 Hz, 1H), 2.18-2.07 (m, 2H), 2.07- |
| | | 1.95 (m, 1H). |
| Example 147 | | |

An analogous method was followed to obtain the following compounds.

| Ex | | | |
|---|---|---|---|
| # | Structure | Starting Material | Characterization |
| 149 | 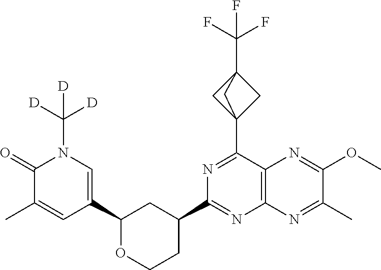 [SMILES: [2H]C([2H])([2H])n1cc([C@H]2C[C@@H](c3nc(C45CC(C(F)(F)F)(C4)C5)c4nc(OC)c(C)nc4n3)CCO2)cc(C)c1=O] | CD₃I 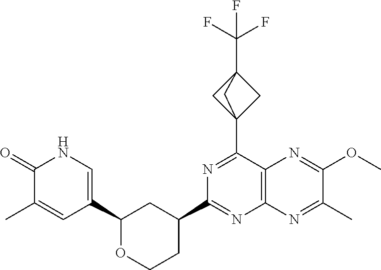 [SMILES: COc1nc2c(C34CC(C(F)(F)F)(C3)C4)nc([C@H]3CCO[C@@H](c4c[nH]c(=O)c(C)c4)C3)nc2nc1C] | Modification: The reaction was ran at 25 °C for 2 hours. The crude was purified by reversed phase-HPLC (100-0% [(0.05% FA)water:ACN]) and lyophilized to give 5- [(2R,4S)-4-[6-methoxy-7- methyl-4-[3- (trifluoromethyl)-1- bicyclo- [1.1.1]pentanyl]pteridin-2- yl]tetrahydropyran-2-yl]-3- methyl-1- (trideuteriomethyl)- pyridin-2-one (Example 149, 21 mg, 0.037 mmol, |
| | | | 27% yield) as a solid. |
| | Example 149 | Example 151 | LCMS: [M + H]⁺ = 519.2; |
| | | | purity = 91% (UV 220 nm); |
| | | | Ret. time = 0.600 min. ¹H |
| | | | NMR (400 MHz, CDCl₃) δ |
| | | | 7.34-7.30 (m, 1H), 7.26 |
| | | | (br s, 1H), 4.35-4.24 (m, |
| | | | 2H), 4.19-4.10 (m, 3H), |
| | | | 3.86-3.74 (m, 1H), 3.49- |
| | | | 3.37 (m, 1H), 2.78-2.71 |
| | | | (m, 3H), 2.67-2.59 (m, |
| | | | 6H), 2.32-2.25 (m, 1H), |
| | | | 2.19-2.15 (m, 3H), 2.15- |
| | | | 2.00 (m, 3H). SFC: 93% |
| | | | Purity. |

### Method 14: Synthesis of Example 132

### Example 132:5-[(2R,4S)-4-[4-[2-fluoro-4-(trifluoromethyl)phenyl]-6-methoxy-7-methyl-pteridin-2-yl]tetrahydropyran-2-yl]-1-(trideuteriomethyl)pyridin-2-one

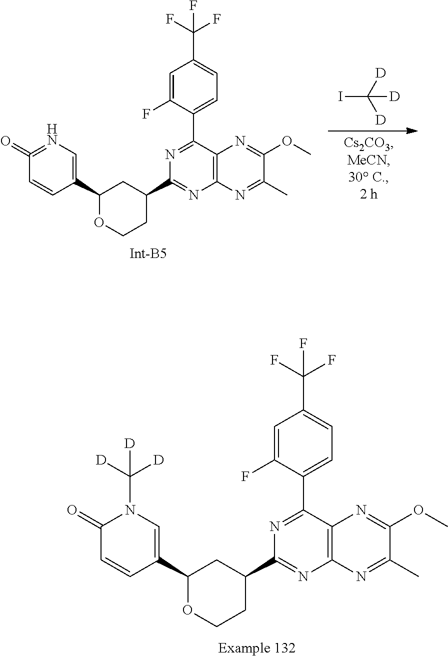

A mixture of 5-[(2R,4S)-4-[4-[2-fluoro-4-(trifluoromethyl)phenyl]-6-methoxy-7-methyl-pteridin-2-yl]tetrahydropyran-2-yl]-1H-pyridin-2-one (Int-B5, 1 equiv., 180 mg, 0.324 mmol) and Cs₂CO₃ (2 equiv., 211 mg, 0.647 mmol) in MeCN (4 mL) was added trideuterio (iodo) methane (5 equiv., 235 mg, 1.62 mmol). The mixture was stirred 30° C. at for 2 h. LCMS showed the starting material was consumed and 88% of the desired mass was detected. The reaction was filtered and concentrated under reduced pressure. The crude product was purified by reversed-phase chromatography (30-70% [water (FA)-ACN], in 10 min) and lyophilized to give 5-[(2R,4S)-4-[4-[2-fluoro-4-(trifluoromethyl)phenyl]-6-methoxy-7-methyl-pteridin-2-yl]tetrahydropyran-2-yl]-1-(trideuteriomethyl)pyridin-2-one (Example 132, 84 mg, 0.152 mmol, 47% yield) as a solid. LCMS: [M+H]⁺=533.2; Purity=97%; Ret. time=0.553 min. ¹H NMR (400 MHz, CDCl₃) δ 7.87 (t, J=7.3 Hz, 1H), 7.63 (d, J=7.9 Hz, 1H), 7.52 (d, J=9.5 Hz, 1H), 7.43-7.36 (m, 2H), 6.61-6.53 (m, 1H), 4.40-4.25 (m, 2H), 4.01 (s, 3H), 3.80 (dt, J=3.9, 11.2 Hz, 1H), 3.61-3.46 (m, 1H), 2.78 (s, 3H), 2.37 (br d, J=13.3 Hz, 1H), 2.25-2.14 (m, 2H), 2.12-2.02 (m, 1H). SFC Ret. time=1.145 min, ee.: 100%.

### Method 15: Synthesis of Example 143 and 151

### Example 143:5-[(2R,4S)-4-[6-methoxy-7-methyl-4-[3-(trifluoromethyl)-1-bicyclo[1.1.1]pentanyl]-pteridin-2-yl]tetrahydropyran-2-yl]-1H-pyridin-2-one

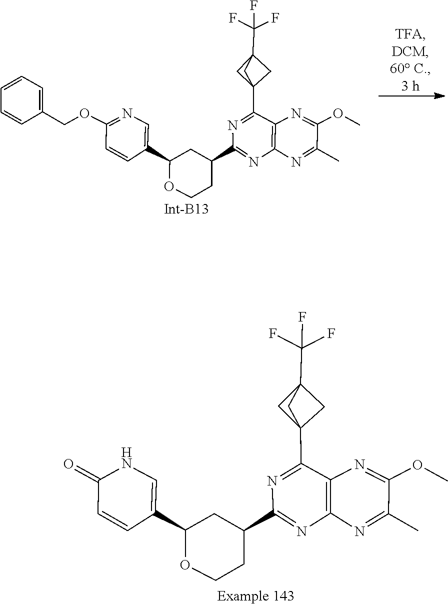 [SMILES: COc1nc2c(C34CC(C(F)(F)F)(C3)C4)nc([C@H]3CCO[C@@H](c4ccc(OCc5ccccc5)nc4)C3)nc2nc1C ; COc1nc2c(C34CC(C(F)(F)F)(C3)C4)nc([C@H]3CCO[C@@H](c4ccc(=O)[nH]c4)C3)nc2nc1C]

A mixture of 2-[(2R,4S)-2-(6-benzyloxy-3-pyridyl)tetrahydropyran-4-yl]-6-methoxy-7-methyl-4-[3-(trifluoromethyl)-1-bicyclo[1.1.1]pentanyl]pteridine (Int-B13, 1 equiv., 180 mg, 0.312 mmol) in DCM (3.6 mL) and TFA (3.6 mL) was stirred at 20° C. for 3 h. LCMS showed a major peak with desired MS. The mixture was added sat. NaHCO₃(aq) to pH=7-8 and extracted with EtOAc (50 mL×3). The organic phase was concentrated under reduced pressure to give 5-[(2R,4S)-4-[6-methoxy-7-methyl-4-[3-(trifluoromethyl)-1-bicyclo[1.1.1]pentanyl]-pteridin-2-yl]tetrahydropyran-2-yl]-1H-pyridin-2-one (Example 143, 100 mg, 0.205 mmol, 66% yield) as a solid, which was used for next step directly. LCMS: [M+H]⁺=488.2; purity=77% (UV 220 nm); Ret. time=0.552 min.

An analogous method was followed to obtain the following compound.

| Structure | Starting Material | Characterization |
|---|---|---|
| 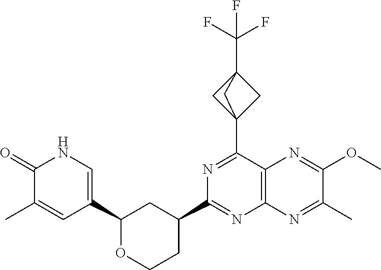 [SMILES: COc1nc2c(C34CC(C(F)(F)F)(C3)C4)nc([C@H]3CCO[C@@H](c4c[nH]c(=O)c(C)c4)C3)nc2nc1C] | 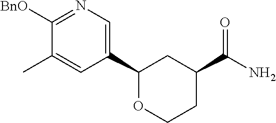 Int-B12 | The mixture was added sat. Na₂CO₃ (aq) to pH = 7-8 and extracted with EtOAc (20 mL × 3) to give 5- [(2R,4S)-4-[6-methoxy-7-methyl-4- [3-(trifluoromethyl)-1- bicyclo[1.1.1]pentanyl]pteridin-2- yl]tetrahydropyran-2-yl]-3-methyl- 1H-pyridin-2-one (Example 151, 80 mg, 0.136 mmol, 100% yield) as a solid (crude). The crude was used for next step directly. LCMS: [M + H]⁺ = 502.3; purity = 85% (UV 220 nm); Ret. time = 0.591 min. |
| Example 151 | | |
| 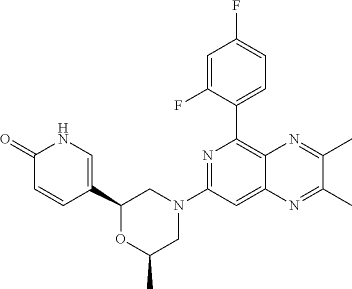 [SMILES: Cc1nc2cc(N3C[C@@H](C)O[C@@H](c4ccc(=O)[nH]c4)C3)nc(-c3ccc(F)cc3F)c2nc1C] | 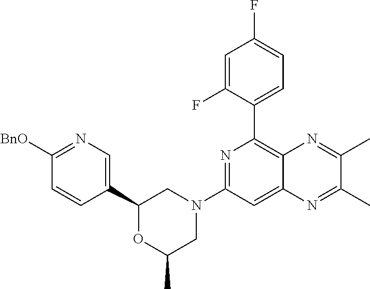 [SMILES: Cc1nc2cc(N3C[C@@H](C)O[C@@H](c4ccc(OCc5ccccc5)nc4)C3)nc(-c3ccc(F)cc3F)c2nc1C] | The reaction was run in a 4 mL vial sealed with a red screw cap equipped with a pressure relief septum at 80 °C for 3 h. The crude product was first purified by flash chromatography (Isco RediSep® column 12 g, using a gradient from 100% hexanes to 100% EtOAc) then further purified by reverse phase flash chromatography (Condition 12, Gradient a) to yield 5- ((2S,6R)-4-(5-(2,4-difluorophenyl)- 2,3-dimethylpyrido[3,4-b]pyrazin-7- yl)-6-methylmorpholin-2-yl)pyridin- 2(1H)-one (Example 186, 4.0 mg, 8.63 μmol, 5% yield). ¹H NMR (CD₃OD, 400 MHz): δ 7.74 (1H, dd, J = 9.4, 2.5 Hz), 7.59-7.63 (1H, m), 7.53 (1H, d, J = 2.4 Hz), 7.03-7.11 |
| | | (2H, m), 7.01 (1H, s), 6.57 (1H, d, J = |
| Example 186 | Int-C19 | 9.4 Hz), 4.56 (1H, d, J = 10.7 Hz), |
| | | 4.47 (1H, d, J = 12.9 Hz), 4.34 (1H, |
| | | d, J = 12.7 Hz), 3.91 (1H, s), 2.82 |
| | | (1H, t, J = 11.7 Hz), 2.68-2.74 (4H, |
| | | m), 2.57 (3H, s), 1.33 (3H, d, J = 6.2 |
| | | Hz). ¹⁹F NMR (CD₃OD, 376 MHz): |
| | | δ −111.5 (1F, dd, 9.4, 17.2 Hz), |
| | | −110.2 (1F, m). |
| | | ESI-MS: [M + H]⁺ = 464.4. |
| 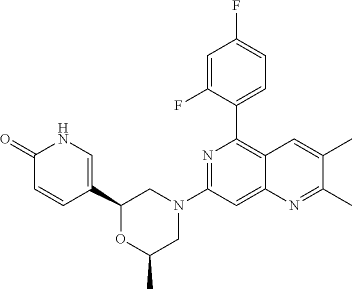 [SMILES: Cc1cc2c(-c3ccc(F)cc3F)nc(N3C[C@@H](C)O[C@@H](c4ccc(=O)[nH]c4)C3)cc2nc1C] | 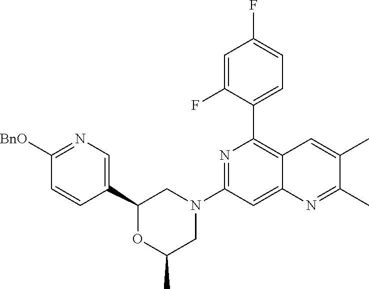 | The procedure of Example 186 was used to obtain 5-((2S,6R)-4-(5-(2,4- difluorophenyl)-2,3-dimethyl-1,6- naphthyridin-7-yl)-6- methylmorpholin-2-yl)pyridin- 2(1H)-one (Example 192). ¹H NMR (CD₃OD, 400 MHz): δ 7.74 (1H, dd, J = 9.4, 2.5 Hz), 7.51-7.60 (3H, m), 7.15 (2H, t, J = 8.7 Hz), 7.00 (1H, s), 6.57 (1H, d, J = 9.4 Hz), 4.56 (1H, d, J = 10.6 Hz), 4.42 (1H, d, J = 12.8 Hz), 4.30 (1H, d, J = 12.6 Hz), 3.91 (1H, s), 2.79 (1H, t, J = 11.7 Hz), 2.70 (1H, d, J = 11.7 Hz), 2.64 (3H, s), 2.35 (3H, s), 1.33 (3H, d, J = 6.2 Hz). ¹⁹F NMR (CD₃OD, 376 MHz): δ −110.6 (1F, m), −112.2 (1F, m). ESI-MS: [M + H]⁺ = 463.4. |
| Example 192 | (2S,6R)-2-(6- | |
| | (benzyloxy)pyridin-3-yl)-4-(5- | |
| | (2,4-difluorophenyl)-2,3- | |
| | dimethyl-1,6-naphthyridin-7- | |
| | yl)-6-methylmorpholine | |
| 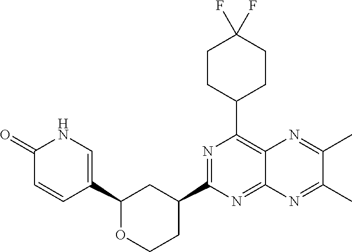 [SMILES: Cc1nc2nc([C@H]3CCO[C@@H](c4ccc(=O)[nH]c4)C3)nc(C3CCC(F)(F)CC3)c2nc1C] | 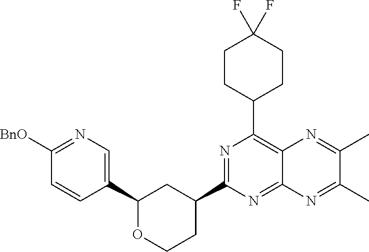 | Modifications: The reaction was run with 1 equiv. Int-D42, 4 equiv. TFA, and DCE solvent at 55 °C for 12 h. The crude was purified first by silica gel column (EtOAc/MeOH = 1:1, R_{f} = 0.4) and then by prep-HPLC (Condition 18, Gradient b) and lyophilized to give 5-[(2R,4S)-4-[4- (4,4-difluorocyclohexyl)-6,7- dimethyl-pteridin-2- yl]tetrahydropyran-2-yl]-1H- pyridin-2-one (Example 224, single enantiomer of known absolute configuration) as a solid. LCMS: [M + H]⁺ = 456.2. ¹H NMR (400 MHz, CDCl₃) δ 1.95-2.20 (m, 9H) |
| | | 2.26-2.37 (m, 3H) 2.80 (d, J = 15.1 |
| | | Hz, 6H) 3.36-3.54 (m, 1H) 3.72- |
| | | 3.85 (m, 1H) 4.16 (br t, J = 11.7 Hz, |
| Example 224 | Int-D42 | 1H) 4.29 (br dd, J = 10.5, 2.8 Hz, 1H) |
| | | 4.35 (br d, J = 10.4 Hz, 1H) 6.59 (d, |
| | | J = 9.4 Hz, 1H) 7.41 (d, J = 1.9 Hz, 1H) |
| | | 7.58 (dd, J = 9.4, 2.3 Hz, 1H) 11.46- |
| | | 12.70 (m, 1H). |
| 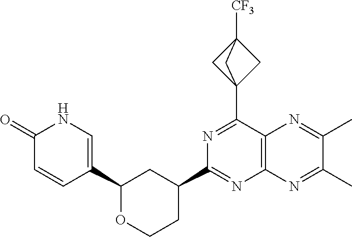 [SMILES: Cc1nc2nc([C@H]3CCO[C@@H](c4ccc(=O)[nH]c4)C3)nc(C34CC(C(F)(F)F)(C3)C4)c2nc1C] | 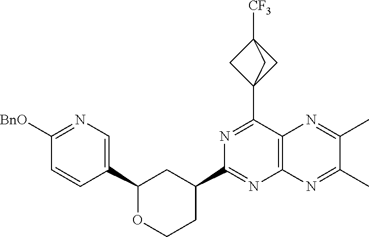 [SMILES: Cc1nc2nc([C@H]3CCO[C@@H](c4ccc(OCc5ccccc5)nc4)C3)nc(C34CC(C(F)(F)F)(C3)C4)c2nc1C] | Modifications: The reaction was run at reflux temperature for 1.5 h and purified by reverse phase chromatography (35-85% MeCN in water (0.1% formic acid) to afford 5- ((2R,4S)-4-(6,7-dimethyl-4-(3- (trifluoromethyl)bicyclo[1.1.1]pentan- 1-yl)pteridin-2-yl)tetrahydro-2H- pyran-2-yl)pyridin-2(1H)-one (Example 34, 1.05 g, 69% yield). ¹H NMR (CDCl₃, 400 MHz): δ 7.56 (1H, dd, J = 9.4, 2.5 Hz), 7.41 (1H, d, J = 2.4 Hz), 6.56 (1H, d, J = 9.4 Hz), 4.32 (1H, d, J = 11.3 Hz), 4.24 (1H, d, J = 11.5 Hz), 3.71-3.77 (1H, m), |
| | | 3.39-3.45 (1H, m), 2.77 (3H, s), 2.73 |
| Example 34 | Int-F9 | (3H, s), 2.61 (6H, s), 2.25 (1H, d, J = |
| | | 13.3 Hz), 2.06-2.11 (2H, m), 1.97 |
| | | (1H, q, J = 12.3 Hz). ESI-MS: |
| | | [M + H]⁺ = 472.3. |

### Method 16: Synthesis of Example 136

### Example 136:5-[(2R)-4-(4-cyclohexyl-6-methoxy-7-methyl-pteridin-2-yl)tetrahydropyran-2-yl]-1-methyl-pyridin-2-one

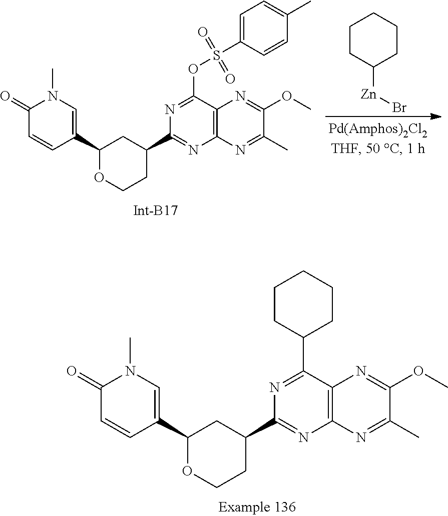

To a solution of [6-methoxy-7-methyl-2-[(2R)-2-(1-methyl-6-oxo-3-pyridyl)tetrahydropyran-4-yl]pteridin-4-yl] 4-methylbenzenesulfonate (Int-B17, 1 equiv., 30 mg, 0.056 mmol) and Pd(Amphos)₂Cl₂ (0.1 equiv., 4.0 mg, 0.006 mmol) in THF (1 mL) was added bromo (cyclohexyl) zinc (3 equiv., 0.33 mL, 0.167 mmol) under N₂, then the mixture was stirred for 1 h at 45° C. LCMS showed raw material was consumed completely and the major peak showed desired MS. The reaction solution was extracted with EtOAc (5 mL×2), the organic layer was washed with saturated brine (5 mL). The combined organic layers were then dried over Na₂SO₄, filtered, and concentrated under reduced pressure to give a crude residue. The residue was purified by prep-TLC (9% MeOH in EtOAc, R_{f}=0.4)(×2) to get 15 mg product. And then the product was purified by prep-HPLC (FA) and freeze-drying to give 5-[(2R)-4-(4-cyclohexyl-6-methoxy-7-methyl-pteridin-2-yl)tetrahydropyran-2-yl]-1-methyl-pyridin-2-one (Example 136, 2.3 mg, 0.005 mmol, 9% yield). LCMS: [M+H]⁺=450.3, purity=97%, uv=220 nm, Ret. Time=0.608 min. ¹H NMR (400 MHz, CDCl₃) δ 1.39 (br t, J=12.5 Hz, 1H), 1.45-1.58 (m, 2H), 1.81 (br s, 2H), 1.92 (br d, J=10.4 Hz, 4H), 2.09-2.17 (m, 2H), 2.32 (br d, J=13.0 Hz, 1H), 2.72 (s, 3H), 3.33-3.48 (m, 1H), 3.56 (s, 3H), 3.70-3.90 (m, 2H), 4.15 (s, 3H), 4.25-4.38 (m, 2H), 6.58 (d, J=9.2 Hz, 1H), 7.38-7.45 (m, 2H).

### Method 17: Synthesis of Example 138

### Example 138:5-[(2R,4S)-4-[4-(4,4-difluorocyclohexyl)-6-methoxy-7-methyl-pteridin-2-yl]tetrahydropyran-2-yl]-1-methyl-pyridin-2-one

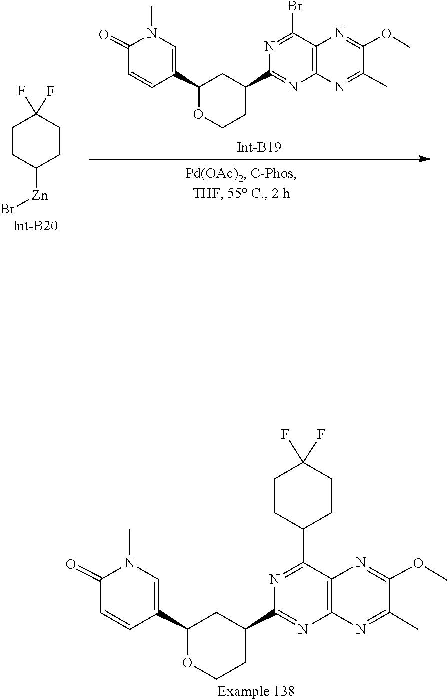

A mixture of 2-bromo-6,7-dimethyl-4-[3-(trifluoromethyl)-1-bicyclo[1.1.1]pentanyl]pteridine (Int-B19, 1 equiv., 10 mg, 0.027 mmol), Pd(OAc)₂ (0.1 equiv., 0.60 mg, 0.003 mmol) and C-Phos (0.2 equiv., 2.3 mg, 0.005 mmol) was purged with N₂ for 3 times. Then THF (0.5 mL) was added, followed by bromo-[2-(1-cyclopropyl-6-oxo-3-pyridyl)tetrahydropyran-4-yl]zinc (Int-B20, 1.2 equiv., 12 mg, 0.032 mmol) solution. The mixture was stirred at 55° C. for 2 h. LCMS showed ~32% of desired product was detected. The mixture was quenched by H₂O (50 mL), extracted with EtOAc (3×80 mL). The combined organic layers was dried over anhydrous Na₂SO₄, filtered, and concentrated under reduced pressure to give a crude product. The mixture was purified with Prep-HPLC (FA) to give the crude product (17 mg) The crude product was purified with Prep-TLC (8% MeOH in EtOAc, R_{f}=0.4) and lyophilized to give 5-[(2R,4S)-4-[4-(4,4-difluorocyclohexyl)-6-methoxy-7-methyl-pteridin-2-yl]tetrahydropyran-2-yl]-1-methyl-pyridin-2-one (Example 138, 4.3 mg, 0.008 mmol) as a solid. LCMS: [M+H]⁺=486.3; Purity=94% (220 nm); Ret. time=0.547 min. ¹H NMR (400 MHz, CDCl₃) δ 7.44-7.37 (m, 2H), 6.61-6.55 (m, 1H), 4.38-4.24 (m, 2H), 4.15 (s, 3H), 3.97-3.73 (m, 2H), 3.56 (s, 3H), 3.49-3.34 (m, 1H), 2.74 (s, 3H), 2.36-2.27 (m, 3H), 2.26-2.17 (m, 2H), 2.13 (br dd, J=4.0, 11.1 Hz, 2H), 2.09-1.97 (m, 5H).

### Method 18: Synthesis of Examples 167-171

### Example 171:5-(4-(2,3-dimethyl-8-(3-(trifluoromethyl) bicyclo[1.1.1]pentan-1-yl) pyrido[2,3-b]pyrazin-6-yl)tetrahydro-2H-pyran-2-yl)-1-methylpyridin-2 (1H)-one

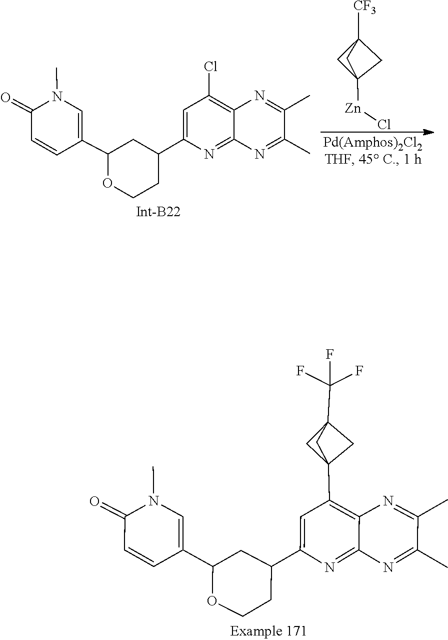

A mixture of 5-[4-(8-chloro-2,3-dimethyl-pyrido[2,3-b]pyrazin-6-yl)tetrahydropyran-2-yl]-1-methyl-pyridin-2-one (Int-B22, 1 equiv., 150 mg, 0.39 mmol), Pd(Amphos)₂Cl₂ (0.05 equiv., 14 mg, 0.0195 mmol) and THF (1 mL) in a sealed bottle under N₂ atmosphere was purged with N₂ three times. Then, chloro-[3-(trifluoromethyl)-1-bicyclo[1.1.1]pentanyl]zinc (1.2 equiv., 110 mg, 0.47 mmol) was added dropwise to the reaction solution at 25° C., then warmed to 45° C. and stirred for 1 hr. LCMS showed ~61% of desired product was detected. The mixture was combined with another batch, then was quenched by H₂O (100 mL), extracted with DCM (3×100 mL), the combined organic layers were dried over anhydrous Na₂SO₄, filtered, and concentrated under reduced pressure to give the crude product (200 mg). The crude product was purified by silica gel column chromatography (0-100% EtOAc in Petroleum ether, then 0-30% MeOH in EtOAc)(TLC, 25% MeOH in EtOAc, desired product R_{f}=0.4) to give crude (Example 171, 100 mg).

### Isolation of Example 167-170

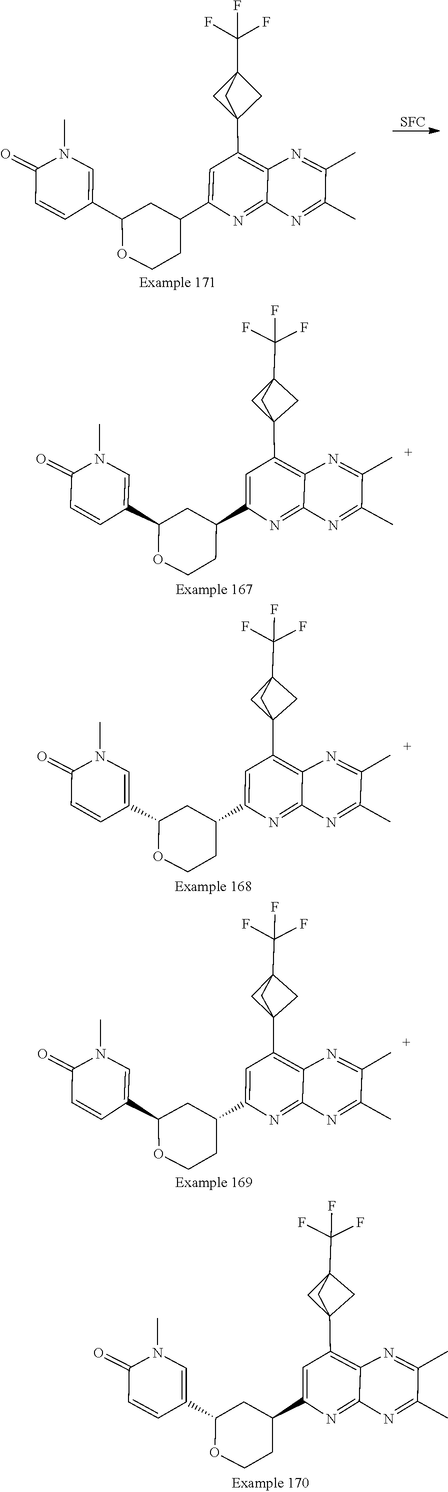

The crude (Example 171) was purified by Prep-HPLC (Condition 7, Gradient c) to give Crude A (40 mg) and Crude B (30 mg, 85% purity).

Crude A (40 mg) was purified by SFC (Condition 11, Gradient d) and lyophilized to get the 1-methyl-5-[(2R,4S)-4-[2,3-dimethyl-8-[3-(trifluoromethyl)-1-bicyclo[1.1.1]pentanyl]pyrido[2,3-b]pyrazin-6-yl]tetrahydropyran-2-yl]pyridin-2-one (Example 167, 9.3 mg, 0.0188 mmol, 6% yield) as a solid and 1-methyl-5-[(2S,4R)-4-[2,3-dimethyl-8-[3-(trifluoromethyl)-1-bicyclo[1.1.1]pentanyl]pyrido[2,3-b]pyrazin-6-yl]tetrahydropyran-2-yl]pyridin-2-one (Example 168, 7.1 mg, 0.015 mmol, 5% yield) as a solid.

Crude B (30 mg, 85% purity) was purified by Prep-HPLC (Condition 7, Gradient d) to give the Crude B (17 mg, 95% purity) which was purified by SFC (Condition 11, Gradient e) and lyophilized to afford 1-methyl-5-[(2R,4R)-4-[2,3-dimethyl-8-[3-(trifluoromethyl)-1-bicyclo-[1.1.1]pentanyl]pyrido[2,3-b]pyrazin-6-yl]tetrahydropyran-2-yl]pyridin-2-one (Example 169, 5.9 mg, 0.012 mmol, 4% yield) as a solid and 1-methyl-5-[(2S,4S)-4-[2,3-dimethyl-8-[3-(trifluoromethyl)-1-bicyclo[1.1.1]pentanyl]pyrido[2,3-b]pyrazin-6-yl]tetrahydropyran-2-yl]pyridin-2-one (Example 170, 8.6 mg, 0.0162 mmol, 5% yield) as a solid.

Example 167: LCMS: [M+H]⁺=485.1; purity=100% (UV 220 nm); Ret. time=0.568 min. HPLC: Ret. time=1.731 min, 98% purity at 220 nm. ¹H NMR (400 MHz, CDCl₃) δ 7.39-7.35 (m, 2H), 7.22 (s, 1H), 6.56 (d, J=10.1 Hz, 1H), 4.29 (br s, 2H), 3.77 (br t, J=11.1 Hz, 1H), 3.54 (s, 3H), 3.35-3.22 (m, 1H), 2.76 (d, J=11.3 Hz, 6H), 2.58 (s, 6H), 2.22-1.94 (m, 4H).

Example 168: LCMS: [M+H]⁺=485.0; purity=100% (UV 220 nm); Ret. time=0.568 min. HPLC: Ret. time=1.725 min, 99% purity at 220 nm. ¹H NMR (400 MHz, CDCl₃) δ 7.38 (s, 2H), 7.22 (s, 1H), 6.56 (d, J=10.1 Hz, 1H), 4.35-4.23 (m, 2H), 3.77 (dt, J=2.1, 11.9 Hz, 1H), 3.54 (s, 3H), 3.36-3.23 (m, 1H), 2.76 (d, J=11.4 Hz, 6H), 2.58 (s, 6H), 2.20-1.94 (m, 4H).

Example 169: LCMS: [M+H]⁺=485.2; purity=95% (UV 220 nm); Ret. time=0.581 min. HPLC: Ret. time=1.623 min, 96% purity at 220 nm. ¹H NMR (400 MHz, CDCl₃) δ 7.42 (br d, J=9.3 Hz, 1H), 7.36 (d, J=1.9 Hz, 1H), 7.27 (s, 1H), 6.59 (d, J=9.4 Hz, 1H), 4.78 (dd, J=2.8, 9.0 Hz, 1H), 3.92 (s, 2H), 3.57-3.51 (m, 4H), 2.78 (d, J=13.3 Hz, 6H), 2.58 (s, 6H), 2.56-2.50 (m, 1H), 2.30-2.12 (m, 3H).

Example 170: LCMS: [M+H]⁺=485.1; purity=99% (UV 220 nm); Ret. time=0.580 min. HPLC: Ret. time=1.625 min, 91% purity at 220 nm. ¹H NMR (400 MHz, CDCl₃) δ 7.43 (dd, J=2.4, 9.3 Hz, 1H), 7.39 (br s, 1H), 7.28 (s, 1H), 6.60 (d, J=9.4 Hz, 1H), 4.79 (dd, J=2.9, 8.6 Hz, 1H), 4.03-3.86 (m, 2H), 3.56 (s, 4H), 2.80 (s, 3H), 2.77 (s, 3H), 2.59 (s, 6H), 2.28-2.13 (m, 4H).

### Method 19. Synthesis of Example 173

### Example 173:5-[(2S,6R)-4-[7-(2,4-difluorophenyl)-2-(dimethylamino) thiazolo[4,5-d]pyrimidin-5-yl]-6-methyl-morpholin-2-yl]-1H-pyridin-2-one

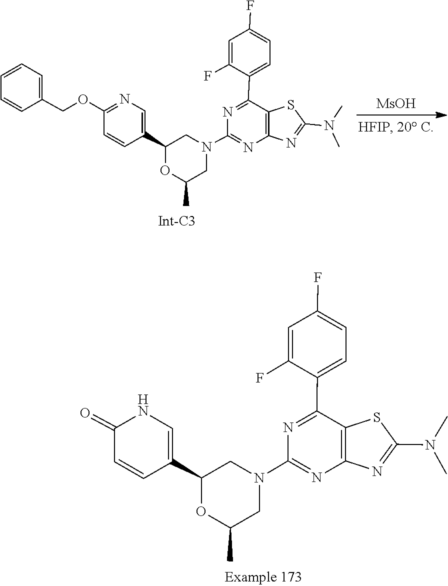 [SMILES: "MsOH" ; C[C@@H]1CN(c2nc(-c3ccc(F)cc3F)c3sc(N(C)C)nc3n2)C[C@H](c2ccc(OCc3ccccc3)nc2)O1 ; C[C@@H]1CN(c2nc(-c3ccc(F)cc3F)c3sc(N(C)C)nc3n2)C[C@H](c2ccc(=O)[nH]c2)O1]

To a solution of 5-[(2S,6R)-2-(6-benzyloxy-3-pyridyl)-6-methyl-morpholin-4-yl]-7-(2,4-difluorophenyl)-N,N-dimethyl-thiazolo[4,5-d]pyrimidin-2-amine (Int-C3, 1 equiv., 90 mg, 0.157 mmol) in 1,1,1,3,3,3-hexafluoropropan-2-ol (1.0 mL) was added MsOH (0.10 mL), and then the mixture was stirred for 12 h at 20° C. and then for another 12 h at 25° C. The reaction solution was added into NaHCO₃ aqueous solution (10 mL), extracted with DCM (10 mL×2) and washed with saturated brine solution. The organics were separated, dried (Na₂SO4) and concentrated to give a residue. ⅓ residue was purified by prep-TLC (DCM/MeOH=20/1, R_{f}=0.4) and lyophilized to obtain 5-[(2S,6R)-4-[7-(2,4-difluorophenyl)-2-(dimethylamino) thiazolo[4,5-d]pyrimidin-5-yl]-6-methyl-morpholin-2-yl]-1H-pyridin-2-one (Example 173) as a solid. LCMS: [M+H]⁺=485.3. ¹H NMR (400 MHz, CDCl₃) δ 1.31 (d, J=6.3 Hz, 3H) 2.65-2.85 (m, 2H) 3.09-3.43 (m, 6H) 3.72-3.87 (m, 1H) 4.40 (dd, J=10.6, 2.4 Hz, 1H) 4.78-4.95 (m, 2H) 6.61 (d, J=9.4 Hz, 1H) 6.95 (ddd, J=10.7, 8.7, 2.5 Hz, 1H) 7.00-7.08 (m, 1H) 7.45 (d, J=2.3 Hz, 1H) 7.57 (dd, J=9.5, 2.5 Hz, 1H) 7.75 (td, J=8.5, 6.6 Hz, 1H).

### Method 20. Synthesis of Example 174

### Example 174:1-amino-5-[(2S,6R)-4-[7-(2,4-difluorophenyl)-2-(dimethylamino) thiazolo[4,5-d]pyrimidin-5-yl]-6-methyl-morpholin-2-yl]pyridin-2-one

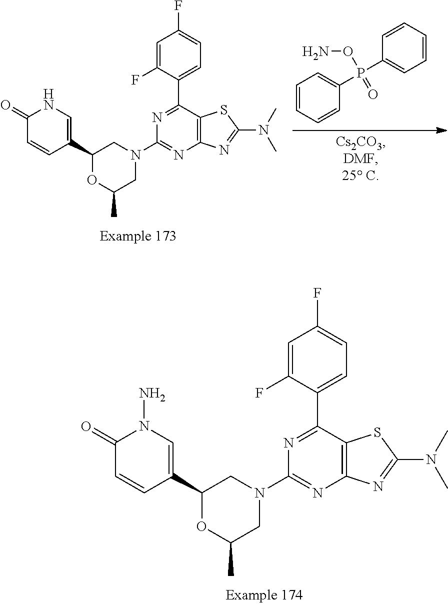

To a mixture of 5-[(2S,6R)-4-[7-(2,4-difluorophenyl)-2-(dimethylamino) thiazolo[4,5-d]pyrimidin-5-yl]-6-methyl-morpholin-2-yl]-1H-pyridin-2-one (Example 173, 1 equiv., 30 mg, 0.0619 mmol) and Cs₂CO₃ (3.5 equiv., 71 mg, 0.217 mmol) in DMF (1 mL) was added O-diphenylphosphorylhydroxylamine (2 equiv., 29 mg, 0.124 mmol) at 0° C. The mixture was then stirred at 20° C. for 45 min. The reaction solution was extracted with DCM (10 mL×2) and washed with 20 mL saturated brine solution. The organics were separated and dried (Na₂SO₄) before concentration to dryness. The residue was then purified by prep-TLC (DCM/MeOH=10/1, R_{f}=0.6) and lyophilized to yield 1-amino-5-[(2S,6R)-4-[7-(2,4-difluorophenyl)-2-(dimethylamino) thiazolo[4,5-d]pyrimidin-5-yl]-6-methyl-morpholin-2-yl]pyridin-2-one (Example 174) as a solid. LCMS: [M+H]⁺=500.2. ¹H NMR (400 MHz, CDCl₃) δ 1.31 (d, J=6.3 Hz, 3H) 2.66-2.85 (m, 2H) 3.77-3.86 (m, 1H) 4.40 (dd, J=10.7, 2.6 Hz, 1H) 4.79-4.94 (m, 2H) 6.66 (d, J=9.3 Hz, 1H) 6.95 (ddd, J=10.7, 8.7, 2.4 Hz, 1H) 7.00-7.06 (m, 1H) 7.5 (dd, J=9.3, 2.4 Hz, 1H) 7.72-7.79 (m, 2H).

An analogous method was followed to obtain the following compounds.

| Structure | Starting Material | Characterization |
|---|---|---|
| 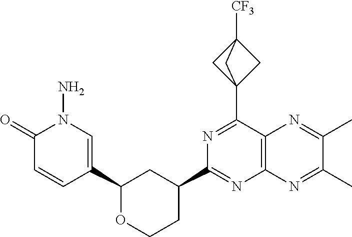 [SMILES: Cc1nc2nc([C@H]3CCO[C@@H](c4ccc(=O)n(N)c4)C3)nc(C34CC(C(F)(F)F)(C3)C4)c2nc1C] | 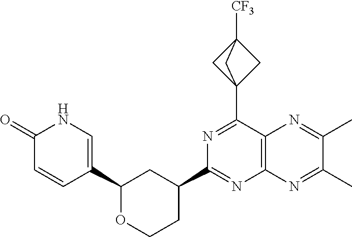 | Modification: The reaction was run in a flame-dried microwave vial charged under argon with 200 mg of starting material. The crude was purified by flash chromatography (12 g silica gel column) using an elution gradient of 0- 10% MeOH in DCM to yield 1-amino- 5-[(2R,4S)-4-[6,7-dimethyl-4-[3- (trifluoromethyl)-1- bicyclo[1.1.1]pentanyl]pteridin-2- yl]tetrahydropyran-2-yl]pyridin-2-one (Example 177, 149 mg, 0.306 mmol, 72% yield) as a solid. ESI-MS: [M + H]⁺ = 487.3. ¹H NMR (CDCl₃, 400 MHz): δ 7.70 (1H, d, J = |
| | | 2.4 Hz), 7.45 (1H, dd, J = 9.4, 2.5 Hz), |
| Example 177 | Example 34 | 6.69 (1H, d, J = 9.5 Hz), 4.34 (1H, d, |
| | | J = 11.4 Hz), 4.26-4.30 (1H, m), 3.74- |
| | | 3.81 (1H, m), 3.46-3.54 (1H, m), 2.80 |
| | | (3H, s), 2.77 (3H, s), 2.65 (6H, s), 2.29- |
| | | 2.32 (1H, m), 2.08-2.15 (2H, m), 1.96- |
| | | 2.05 (1H, m). ¹⁹F NMR (CDCl₃, 376 |
| | | MHz): δ −73.1 (3F, s). |
| 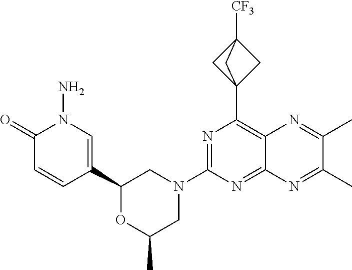 [SMILES: Cc1nc2nc(N3C[C@@H](C)O[C@@H](c4ccc(=O)n(N)c4)C3)nc(C34CC(C(F)(F)F)(C3)C4)c2nc1C] | 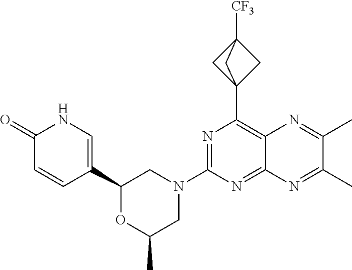 [SMILES: Cc1nc2nc(N3C[C@@H](C)O[C@@H](c4ccc(=O)[nH]c4)C3)nc(C34CC(C(F)(F)F)(C3)C4)c2nc1C] | Modification: The reaction with 5- [(2S,6R)-4-[6,7-dimethyl-4-[3- (trifluoromethyl)-1- bicyclo[1.1.1]pentanyl]pteridin-2-yl]- 6-methyl-morpholin-2-yl]-1H-pyridin- 2-one (Int-C16) was run at 40 °C for 3 h. The orange residue was purified firstly, by flash chromatography (6 g C18 column) using an elution gradient of 10-90% ACN in 0.1% FA (aq.), secondly, by prep-HPLC (Condition 10, Gradient a) to yield 1-amino-5- [(2S,6R)-4-[6,7-dimethyl-4-[3- (trifluoromethyl)-1- bicyclo[1.1.1]pentanyl]pteridin-2-yl]- 6-methyl-morpholin-2-yl]pyridin-2- one (Example 184, 1.6 mg, 320 μmol, |
| | | 2% yield) as a solid. ESI-MS: |
| Example 184 | Int-C16 | [M + H]⁺ = 502.4. |
| | | ¹H NMR: (CD₃OD, 400 MHz): δ 7.89 |
| | | (1H, s), 7.63 (1H, d, J = 9.1 Hz), 6.64 |
| | | (1H, d, J = 9.2 Hz), 4.94 (2H, dd, J = |
| | | 30.1, 13.3 Hz), 4.46 (1H, dd, J = 10.7, |
| | | 2.6 Hz), 3.80 (1H, ddd, J = 10.6, 6.2, |
| | | 2.7 Hz), 2.92 (1H, dd, J = 13.3, 10.8 |
| | | Hz), 2.81 (1H, dd, J = 13.3, 10.7 Hz), |
| | | 2.67 (3H, s), 2.65 (3H, s), 2.59 (6H, s), |
| | | 1.32 (3H, d, J = 6.2 Hz). ¹⁹F NMR |
| | | (CD₃OD, 376 MHz): δ −74.8 (3F, s). |
| 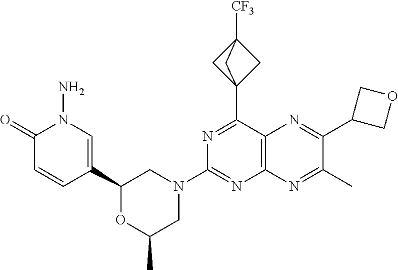 [SMILES: Cc1nc2nc(N3C[C@@H](C)O[C@@H](c4ccc(=O)n(N)c4)C3)nc(C34CC(C(F)(F)F)(C3)C4)c2nc1C1COC1] | 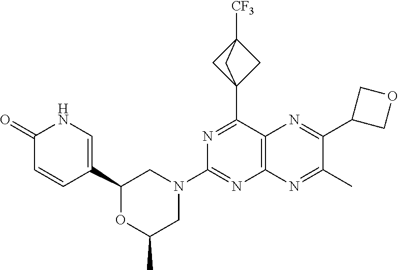 [SMILES: Cc1nc2nc(N3C[C@@H](C)O[C@@H](c4ccc(=O)[nH]c4)C3)nc(C34CC(C(F)(F)F)(C3)C4)c2nc1C1COC1] | Modification: The reaction was started at 0 °C in an ice water bath and slowly warmed up to r.t. with stirring over 3.5 h. The crude was purified by flash chromatography (Isco RediSep column 12 g, using a gradient from 0 to 2% MeOH in DCM) to yield 1-amino-5- [(2S,6R)-6-methyl-4-[7-methyl-6- (oxetan-3-yl)-4-[3-(trifluoromethyl)-1- bicyclo[1.1.1]pentanyl]pteridin-2- yl]morpholin-2-yl]pyridin-2-one (Example 185, 55% yield) as a solid. ¹H NMR (CDCl₃, 400 MHz): δ 7.76 (s, 1H), 7.46 (d, J = 9.0 Hz, 1H), 6.67-6.69 (m, 1H), 5.08 (t, J = 7.6 Hz, 4H), 4.99- 5.04 (m, 1H), 4.92-5.00 (m, 1H), 4.60- 4.68 (m, 1H), 4.39 (dd, J = 10.7, 2.6 Hz, |
| | | 1H), 3.75-3.84 (m, 1H), 2.77-2.95 (m, |
| Example 185 | Int-C18 | 3H), 2.62 (s, 6H), 2.55 (s, 2H), 1.34 (d, |
| | | J = 6.1 Hz, 3H). ¹⁹F NMR (CDCl₃, 376 |
| | | MHz): δ −72.9 (s, 3F). ESI-MS: |
| | | [M + H]⁺ = 544.3. |
| 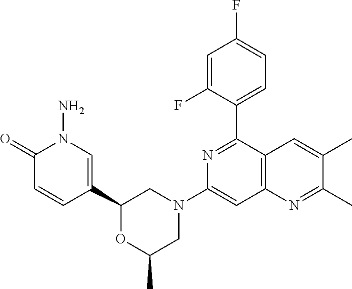 [SMILES: Cc1cc2c(-c3ccc(F)cc3F)nc(N3C[C@@H](C)O[C@@H](c4ccc(=O)n(N)c4)C3)cc2nc1C] | 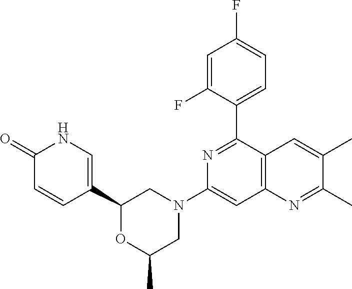 [SMILES: Cc1cc2c(-c3ccc(F)cc3F)nc(N3C[C@@H](C)O[C@@H](c4ccc(=O)[nH]c4)C3)cc2nc1C] | Modification: A flame-dried microwave vial was charged with 25 mg of starting material Example 192 and the reagents, chilled to 0 °C, and then charged with DMF (1.1 mL) under stirring. The reation was warmed up to r.t. for 1 h. The crude material was purified by reverse phase chromatography (Condition 13, Gradient a) to afford 1-amino-5- [(2S,6R)-4-[5-(2,4-difluorophenyl)- 2,3-dimethyl-1,6-naphthyridin-7-yl]-6- methyl-morpholin-2-yl]pyridin-2-one (Example 193, 12.2 mg, 25.5 μmol, 47% yield) as a solid. ¹H NMR (400 MHz, CDCl₃): δ 7.76 (s, 1H), 7.46 (d, J = 9.0 Hz, 1H), 6.67-6.69 (m, 1H), 5.08 (t, J = 7.6 Hz, 5H), 4.99-5.04 (m, 1H), |
| | | 4.92-5.00 (m, 1H), 4.60-4.68 (m, 1H), |
| Example 193 | Example 192 | 4.39 (dd, J = 10.7, 2.6 Hz, 1H), 3.75- |
| | | 3.84 (m, 1H), 2.77-2.95 (m, 3H), 2.62 |
| | | (s, 6H), 2.55 (s, 2H), 1.34 (d, J = 6.1 |
| | | Hz, 3H). ¹⁹F NMR (CDCl₃, 376 MHz): |
| | | δ −109.6 (s, 2F). ESI-MS: [M + H]⁺ = |
| | | 533.3. |
| 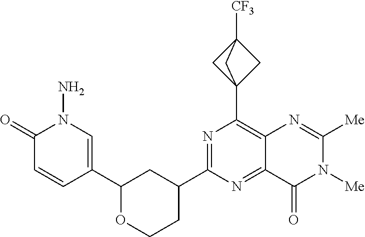 [SMILES: Cc1nc2c(C34CC(C(F)(F)F)(C3)C4)nc(C3CCOC(c4ccc(=O)n(N)c4)C3)nc2c(=O)n1C] | 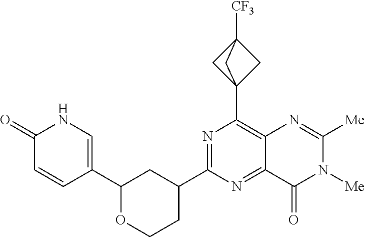 [SMILES: Cc1nc2c(C34CC(C(F)(F)F)(C3)C4)nc(C3CCOC(c4ccc(=O)[nH]c4)C3)nc2c(=O)n1C] | Modifications: The reaction was run with 3 equiv. each of Cs₂CO₃. The crude residue was purified by flash chromatography (4 g SiO₂) using an elution gradient of 0-10% MeOH in DCM to yield 2,3-dimethyl-6-[rac- (2R,4S)-2-(1-amino-6-oxo-3- pyridyl)tetrahydropyran-4-yl]-8-[3- (trifluoromethyl)-1- bicyclo[1.1.1]pentanyl]pyrimido[5,4- d]pyrimidin-4-one (Example 218, 3.2 mg, 6.4 μmol, 34% yield) as a solid. ESI-MS: [M + H]⁺ = 503.3. ¹H NMR (CDCl₃, 400 MHz): δ 7.67 (d, J = 2.5 Hz, 1H), 7.41 (dd, J = 9.4, 2.5 Hz, 1H), |
| | | 6.64 (d, J = 9.4 Hz, 1H), 4.31-4.24 (m, |
| Example 218 | Example 216 | 2H), 3.74 (td, J = 11.9, 2.3 Hz, 1H), |
| | | 3.66 (s, 3H), 3.47 (tt, J = 11.9, 3.8 Hz, |
| | | 1H), 2.65 (s, 3H), 2.57 (s, 6H), 2.21- |
| | | 2.18 (m, 1H), 2.13-2.05 (m, 1H), 2.01 |
| | | (d, J = 13.5 Hz, 1H), 1.93 (dd, J = 24.8, |
| | | 11.8 Hz, 1H). ¹⁹F NMR (CDCl₃, 376 |
| | | MHz): δ −73.0 (s, 3F). |
| 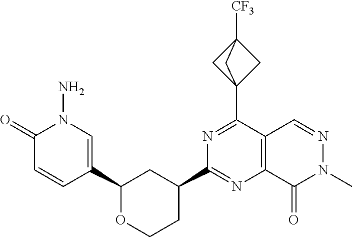 [SMILES: Cn1ncc2c(C34CC(C(F)(F)F)(C3)C4)nc([C@H]3CCO[C@@H](c4ccc(=O)n(N)c4)C3)nc2c1=O] | 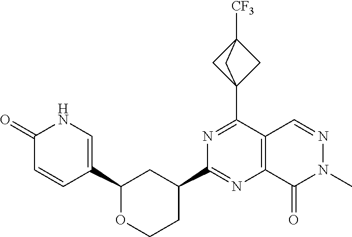 | The crude mixture was purified by silica gel flash column chromatography using an elution gradient of 1-10% MeOH in DCM to yield 2-((2R,4S)-2- (1-amino-6-oxo-1,6-dihydropyridin-3- yl)tetrahydro-2H-pyran-4-yl)-7- methyl-4-(3- (trifluoromethyl)bicyclo[1.1.1]pentan- 1-yl)pyrimido[4,5-d]pyridazin-8(7H)- one (Example 229, 12 mg, 0.024 mmol, 76% yield) as a solid. ESI-MS: [M + H]⁺ = 489.3. ¹H NMR (400 MHz, CDCl₃): δ 8.44 (s, 1H), 7.66 (d, J = 2.3 Hz, 1H), 7.39 (dd, J = 9.3, 2.6 Hz, 1H), 6.62 (d, J = 9.4 Hz, 1H), |
| | | 5.17 (s, 2H), 4.31-4.24 (m, 2H), 3.89 (s, |
| Example 229 | Example 227 | 3H), 3.74 (td, J = 11.8, 2.5 Hz, 1H), |
| | | 3.58-3.53 (m, 1H), 2.61 (s, 6H), 2.21 |
| | | (d, J = 13.0 Hz, 1H), 2.12-2.00 (m, 2H), |
| | | 1.92 (dd, J = 24.6, 12.0 Hz, 1H), 1.24 |
| | | (s, 1H). ¹⁹F NMR (376 MHz, CDCl₃): |
| | | δ −73.0 (s, 3F). |
| 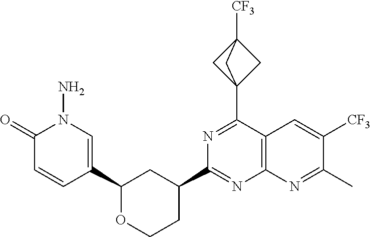 [SMILES: Cc1nc2nc([C@H]3CCO[C@@H](c4ccc(=O)n(N)c4)C3)nc(C34CC(C(F)(F)F)(C3)C4)c2cc1C(F)(F)F] | 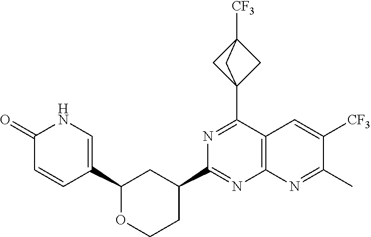 | The crude mixture was purified by silica gel flash column chromatography (1-7% MeOH in DCM) to yield 1- amino-5-((2R,4S)-4-(7-methyl-6- (trifluoromethyl)-4-(3- (trifluoromethyl)bicyclo[1.1.1]pentan- 1-yl)pyrido[2,3-d]pyrimidin-2- yl)tetrahydro-2H-pyran-2-yl)pyridin- 2(1H)-one (Example 332, 67 mg, 0.12 mmol, 71% yield) as a solid. ESI-MS: [M + H]⁺ = 540.3. ¹H NMR (400 MHz, DMSO-d₆): δ 8.84 (s, 1H), 7.68 (d, J = 2.5 Hz, 1H), 7.40 (dd, J = 9.1, 2.5 Hz, 1H), 6.41 (d, J = 9.4 Hz, 1H), 6.12 (s, |
| | | 2H), 4.38 (d, J = 9.8 Hz, 1H), 4.12 (dd, |
| Example 332 | Example 330 | J = 11.3, 3.5 Hz, 1H), 3.72-3.66 (m, |
| | | 1H), 3.39 (tt, J = 11.8, 3.6 Hz, 1H), 2.81 |
| | | (s, 3H), 2.65 (s, 6H), 2.18 (d, J = 13.0 |
| | | Hz, 1H), 2.04-2.00 (m, 1H), 1.92-1.72 |
| | | (m, 2H). ¹⁹F NMR (376 MHz, DMSO- |
| | | d6): δ −60.8 (s, 3F), −71.2 (s, 3F). |

### Method 21: Synthesis of Example 175

### Example 175:5-[(2S,6R)-4-[7-(2,4-difluorophenyl)-2-(dimethylamino) thiazolo[4,5-d]pyrimidin-5-yl]-6-methyl-morpholin-2-yl]-1-(methylamino)pyridin-2-one

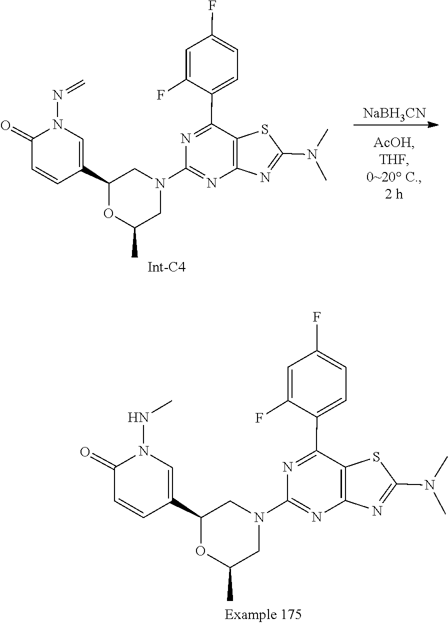

To a solution of 5-[(2S,6R)-4-[7-(2,4-difluorophenyl)-2-(dimethylamino) thiazolo[4,5-d]pyrimidin-5-yl]-6-methyl-morpholin-2-yl]-1-(methyleneamino)pyridin-2-one (Int-C4, 1 equiv., 60 mg, 0.117 mmol) in THF (2 mL) was added AcOH (2 equiv., 32 mg, 0.235 mmol) to adjust pH to ~4. NaBH₃CN (2.5 equiv., 18 mg, 0.293 mmol) was added at 0° C., and then the mixture was stirred for 1 h at 20° C. The reaction solution was added into NH₄Cl aqueous solution (10 mL) and then extracted with DCM (15 mL×3). The organics were washed with 10 mL saturated brine solution, separated, and dried (Na₂SO₄) before concentration to dryness. The crude was then purified by prep-HPLC (Condition 3, Gradient c) and lyophilized to yield 25 mg residue, which was purified by prep-TLC (DCM/MeOH=7/1, R_{f}=0.4) to yield 5-[(2S,6R)-4-[7-(2,4-difluorophenyl)-2-(dimethylamino) thiazolo[4,5-d]pyrimidin-5-yl]-6-methyl-morpholin-2-yl]-1-(methylamino)pyridin-2-one (Example 175, 2.2 mg, 0.00396 mmol, 3% yield) as a solid. LCMS: [M+H]⁺=514.1; purity=88.5% (UV 220 nm); Ret. time=0.611 min. 1H NMR (400 MHz, CD₃OD) δ 1.31 (br s, 3H) 1.56-1.66 (m, 1H) 1.93-2.09 (m, 1H) 2.66-2.91 (m, 5H) 3.28 (br s, 6H) 3.81 (ddd, J=10.6, 6.4, 2.8 Hz, 1H) 4.47 (dd, J=10.7, 2.4 Hz, 1H) 4.74 (br d, J=12.4 Hz, 1H) 6.7 (d, J=9.4 Hz, 1H) 7.09-7.19 (m, 2H) 7.64 (dd, J=9.3, 2.4 Hz, 1H) 7.75-7.89 (m, 2H).

### Method 22: Synthesis of Example 176

### Example 176:5-[(2S,6R)-4-[7-(2,4-difluorophenyl)-2-(dimethylamino) thiazolo[4,5-d]pyrimidin-5-yl]-6-methyl-morpholin-2-yl]-1-methyl-pyridin-2-one

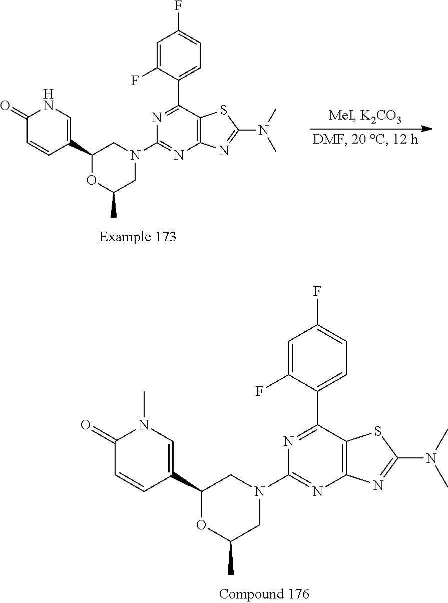 [SMILES: C[C@@H]1CN(c2nc(-c3ccc(F)cc3F)c3sc(N(C)C)nc3n2)C[C@H](c2ccc(=O)[nH]c2)O1 ; C[C@@H]1CN(c2nc(-c3ccc(F)cc3F)c3sc(N(C)C)nc3n2)C[C@H](c2ccc(=O)n(C)c2)O1]

To a solution of 5-[(2S,6R)-4-[7-(2,4-difluorophenyl)-2-(dimethylamino) thiazolo[4,5-d]pyrimidin-5-yl]-6-methyl-morpholin-2-yl]-1H-pyridin-2-one (Example 173, 1 equiv., 20 mg, 0.0413 mmol) and K₂CO₃ (2 equiv., 11 mg, 0.0826 mmol) in DMF (0.50 mL) was added MeI (1.5 equiv., 8.8 mg, 0.0619 mmol), and then the mixture was stirred at 20° C. for 12 h. The reaction solution was added into NH₄Cl aqueous solution (5 mL), extracted with ethyl acetate (5 mL×3) and washed with 10 mL saturated brine solution. The organics were then separated and concentrated to dryness. The residue was then purified by prep-HPLC (Condition 3) and lyophilized to give 5-[(2S,6R)-4-[7-(2,4-difluorophenyl)-2-(dimethylamino) thiazolo[4,5-d]pyrimidin-5-yl]-6-methyl-morpholin-2-yl]-1-methyl-pyridin-2-one (Example 176, 7.2 mg, 0.0142 mmol, 34% yield) as a solid. LCMS: [M+H]⁺=499.3; purity=100% (UV 220 nm); Ret. time=0.548 min. ¹H NMR (400 MHz, CDCl₃ δ 1.32 (d, J=6.1 Hz, 3H) 2.65-2.87 (m, 2H) 3.12-3.47 (m, 6H) 3.57 (s, 3H) 3.81 (ddd, J=10.5, 6.3, 2.6 Hz, 1H) 4.38 (dd, J=10.7, 2.6 Hz, 1H) 4.73-4.97 (m, 2H) 6.59 (d, J=9.3 Hz, 1H) 6.91-7.06 (m, 2H) 7.39-7.47 (m, 2H) 7.75 (td, J=8.4, 6.5 Hz, 1H).

An analogous method was followed to obtain the following compounds.

| Structure | Starting Material | Characterization |
|---|---|---|
| 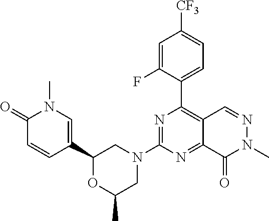 [SMILES: C[C@@H]1CN(c2nc(-c3ccc(C(F)(F)F)cc3F)c3cnn(C)c(=O)c3n2)C[C@H](c2ccc(=O)n(C)c2)O1] | 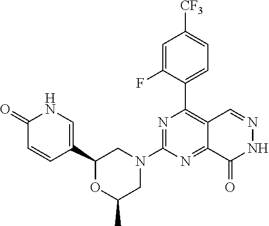 | Modification: The reaction was run with 3 equiv. K₂CO₃ and 5 equiv. MeI at 50 °C for 2 h. The crude was purified by prep-HPLC (Condition 18, Gradient a) and lyophilized to afford 4-[2-fluoro- 4-(trifluoromethyl)phenyl]-7-methyl- 2-[(2R,6S)-2-methyl-6-(1-methyl-6- oxo-3-pyridyl)morpholin-4- yl]pyrimido[4,5-d]pyridazin-8-one (Example 221, 37 mg, 0.0676 mmol, 85% yield, a single enantiomer of known absolute configuration) as a solid. LCMS: [M + H]⁺ = 531.2. ¹H NMR (400 MHz, CDCl₃) δ 1.36 (br d, |
| Example 221 | Int-D25 | J = 14.3 Hz, 3H) 2.80-3.04 (m, 2H) 3.53- |
| | | 3.63 (m, 3H) 3.78-3.90 (m, 4H) 4.38 |
| | | (br s, 1H) 4.80-4.95 (m, 1H) 5.08- |
| | | 5.25 (m, 1H) 6.63 (br s, 1H) 7.34-7.52 |
| | | (m, 2H) 7.57 (br d, J = 9.3 Hz, 1H) 7.63- |
| | | 7.79 (m, 3H). |
| 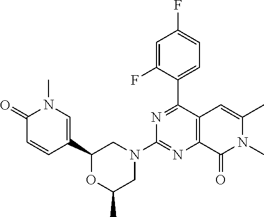 | 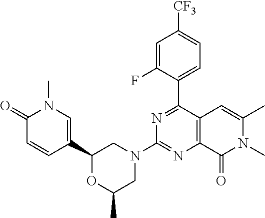 | Modification: The reaction was run with 3 equiv. K₂CO₃ and 10 equiv. MeI at 50 °C for 18 h. The crude was purified by prep-HPLC (Column: C18 150 × 40 mm × 15 μm; Mobile Phase A: water (0.3% FA); Mobile Phase B: ACN) to obtain the formic acid salt of 4-(2,4-difluorophenyl)-6,7-dimethyl-2- [(2R,6S)-2-methyl-6-(1-methyl-6-oxo- 3-pyridyl)morpholin-4-yl]pyrido[3,4- d]pyrimidin-8-one (Example 222, 28.2 mg, 0.0516 mmol, 31% yield, single enantiomer of known absolute configuration) as a solid. LCMS: |
| Example 222 | Int-D35 | [M + H]⁺ = 494.2. ¹H NMR (400 MHz, |
| | | DMSO-d₆) δ 7.78 (d, J = 1.5 Hz, 1H), |
| | | 7.70-7.61 (m, 1H), 7.55-7.44 (m, 2H), |
| | | 7.30 (dt, J = 2.3, 8.4 Hz, 1H), 6.40 (d, J = |
| | | 9.3 Hz, 1H), 6.00 (d, J = 2.4 Hz, 1H), |
| | | 4.72-4.53 (m, 2H), 4.38 (dd, J = 2.2, |
| | | 10.8 Hz, 1H), 3.80-3.70 (m, 1H), 3.50 |
| | | (s, 3H), 3.43 (s, 3H), 2.95 (dd, J = 11.1, |
| | | 13.0 Hz, 1H), 2.73 (dd, J = 10.8, 13.1 |
| | | Hz, 1H), 2.31 (s, 3H), 1.23 (br d, J = 6.0 |
| | | Hz, 3H); ¹⁹F NMR (376 MHz, DMSO- |
| | | d₆) δ −107.35, −110.12. |
| 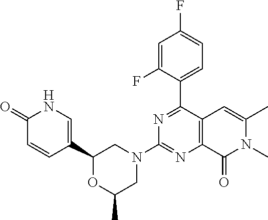 [SMILES: Cc1cc2c(-c3ccc(F)cc3F)nc(N3C[C@@H](C)O[C@@H](c4ccc(=O)[nH]c4)C3)nc2c(=O)n1C] | 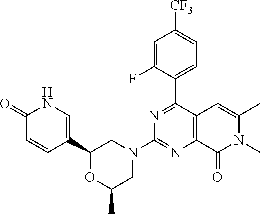 [SMILES: Cc1cc2c(-c3ccc(C(F)(F)F)cc3F)nc(N3C[C@@H](C)O[C@@H](c4ccc(=O)[nH]c4)C3)nc2c(=O)n1C] | Modification: The reaction was run with 3 equiv. K₂CO₃ and 10 equiv. MeI at 50 °C for 18 h. The crude was purified by prep-HPLC (Column: Phenomenex luna C18 150 × 25 mm × 10 μm; Mobile Phase A: water (0.1% FA); Mobile Phase B: ACN) to obtain 4-[2-fluoro-4- (trifluoromethyl)phenyl]-6,7-dimethyl- 2-[(2R,6S)-2-methyl-6-(1-methyl-6- oxo-3-pyridyl)morpholin-4- yl]pyrido[3,4-d]pyrimidin-8-one (Example 223, 85 mg, 0.154 mmol, 54% yield as a single enantiomer of |
| Example 223 | Int-D38 | known absolute configuration) as a |
| | | solid. LCMS: [M + H]⁺ = 544.3. ¹H |
| | | NMR (400 MHz, DMSO-d₆) δ 7.94 (d, |
| | | J = 9.8 Hz, 1H), 7.86-7.73 (m, 3H), |
| | | 7.51 (dd, J = 2.4, 9.3 Hz, 1H), 6.40 (d, |
| | | J = 9.3 Hz, 1H), 6.03 (d, J = 2.1 Hz, |
| | | 1H), 4.64 (br dd, J = 6.9, 8.7 Hz, 2H), |
| | | 4.39 (dd, J = 2.5, 10.8 Hz, 1H), 3.85- |
| | | 3.71 (m, 1H), 3.54-3.48 (m, 3H), 3.46- |
| | | 3.39 (m, 3H), 3.02-2.91 (m, 1H), 2.79- |
| | | 2.65 (m, 1H), 2.32 (s, 3H), 1.24 (br d, |
| | | J = 6.0 Hz, 3H). ee: ~100%. |

### Method 23: Synthesis of Example 178

### Example 178:1-(dimethylamino)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one

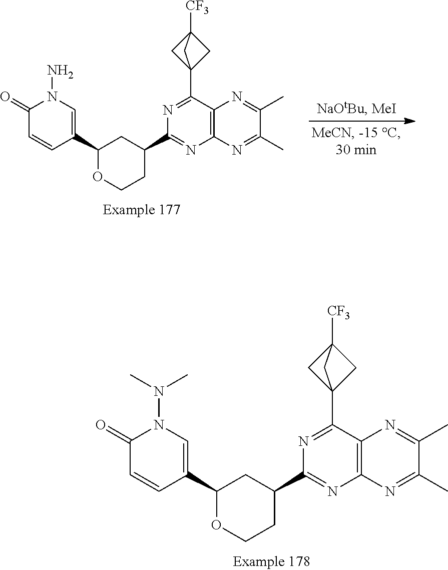 [SMILES: Cc1nc2nc([C@H]3CCO[C@@H](c4ccc(=O)n(N)c4)C3)nc(C34CC(C(F)(F)F)(C3)C4)c2nc1C ; Cc1nc2nc([C@H]3CCO[C@@H](c4ccc(=O)n(N(C)C)c4)C3)nc(C34CC(C(F)(F)F)(C3)C4)c2nc1C]

NaOtBu (2.0 M in THF, 2 equiv., 514 μL, 1.03 mmol) was added dropwise to a solution of 1-amino-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 177, 1 equiv., 250 mg, 0.51 mmol) in MeCN (5 mL) at-15° C. under argon. MeI (1.0 M in MeCN, 1.56 equiv., 50 μL, 0.800 mmol) was added and the reaction mixture was stirred for 20 min at −15° C. under argon atmosphere. The reaction mixture was warmed up to 0° C. and water was added. The aqueous phase was extracted with DCM (3×20 mL). The combined organic layers were washed with brine, dried over anhydrous Na₂SO₄, filtered and concentrated under reduced pressure. The resulting oil was purified, first, by reverse phase flash column chromatography (Condition 9, Gradient a), and then, second, by prep-HPLC (Condition 10, Gradient a) to yield 1-(dimethylamino)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 178, 31 mg, 0.061 mmol, 12% yield) as a powder. ESI-MS: [M+H]⁺=515.4. ¹H NMR (CDCl₃, 400 MHz): δ 7.52 (1H, d, J=2.4 Hz), 7.37 (1H, dd, J=9.4, 2.5 Hz), 6.56 (1H, d, J=9.4 Hz), 4.26-4.31 (2H, m), 3.79 (1H, ddd, J=11.7, 11.2, 3.2 Hz), 3.40-3.47 (1H, m), 3.03 (6H, s), 2.81 (3H, s), 2.77 (3H, s), 2.65 (6H, s), 2.29-2.33 (1H, m), 2.01-2.16 (3H, m). ¹⁹F NMR (CDCl₃, 376 MHz): δ −73.1 (3F, s).

### Method 24: Synthesis of Example 179

### Example 179:5-[(2R,4S)-4-[6,7-dimethyl-4-[3-(trifluoromethyl)-1-bicyclo[1.1.1]pentanyl]pteridin-2-yl]tetrahydropyran-2-yl]-1-(methylamino)pyridin-2-one

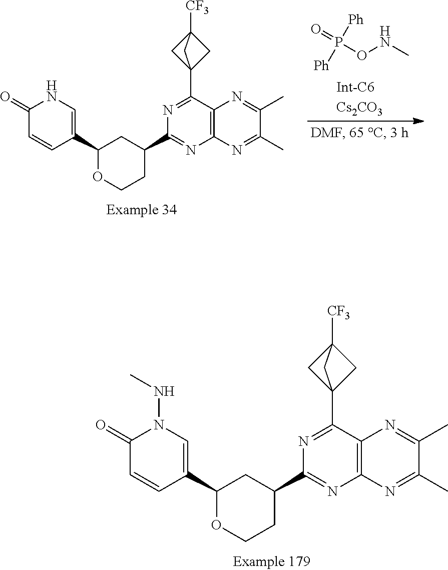 [SMILES: Cc1nc2nc([C@H]3CCO[C@@H](c4ccc(=O)[nH]c4)C3)nc(C34CC(C(F)(F)F)(C3)C4)c2nc1C ; CNOP(=O)(c1ccccc1)c1ccccc1 ; CNn1cc([C@H]2C[C@@H](c3nc(C45CC(C(F)(F)F)(C4)C5)c4nc(C)c(C)nc4n3)CCO2)ccc1=O]

A flame-dried MW vial was charged with 5-[(2R,4S)-4-[6,7-dimethyl-4-[3-(trifluoromethyl)-1-bicyclo[1.1.1]pentanyl]pteridin-2-yl]tetrahydropyran-2-yl]-1H-pyridin-2-one (Example 34, 1 equiv., 100 mg, 0.21 mmol), cesium carbonate (4 equiv., 276 mg, 0.85 mmol), N-diphenylphosphoryloxymethanamine (Int-C6, 4 equiv., 210 mg, 0.85 mmol), DMF (2 mL), the vial was sealed and the reaction mixture was stirred at 65° C. for 2 h. The reaction mixture was cooled to 0° C. and water was added. The aqueous suspension formed was extracted with EtOAc (4×20 mL). The combined organic phases were washed with water and brine. The organic layer was dried with anhydrous Na₂SO₄, filtered and evaporated under reduced pressure. The resulting crude was purified first, by flash chromatography (12 g SiO₂ column) using an elution gradient of 0-10% MeOH in DCM, and second, by reverse phase flash chromatography (Condition 11, Graident a) to yield 5-[(2R,4S)-4-[6,7-dimethyl-4-[3-(trifluoromethyl)-1-bicyclo[1.1.1]pentanyl]pteridin-2-yl]tetrahydropyran-2-yl]-1-(methylamino)pyridin-2-one (Example 179, 5.0 mg, 10.0 μmol, 5% yield) as a solid. ESI-MS: [M+H]⁺=501.3. ¹H NMR (400 MHz, CDCl₃): δ 7.65 (1H, d, J=2.4 Hz), 7.45 (1H, dd, J=9.3, 2.5 Hz), 6.72 (1H, d, J=9.4 Hz), 4.34-4.37 (1H, m), 4.26-4.31 (1H, m), 3.76-3.83 (1H, m), 3.46-3.53 (1H, m), 2.81 (6H, m), 2.77 (3H, s), 2.66 (6H, s), 2.31-2.35 (1H, m), 2.12-2.16 (2H, m), 1.98-2.07 (1H, m). ¹⁹F NMR (CDCl₃, 376 MHz): δ −73.1 (3F, s).

An analogous method was followed to obtain the following compounds.

| Structure | Starting Material | Characterization |
|---|---|---|
| 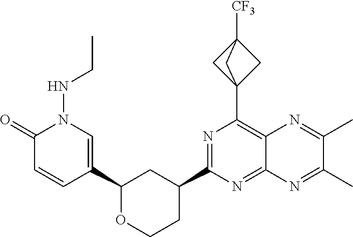 [SMILES: CCNn1cc([C@H]2C[C@@H](c3nc(C45CC(C(F)(F)F)(C4)C5)c4nc(C)c(C)nc4n3)CCO2)ccc1=O] | 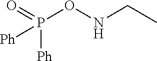 Int-C8 | Modification: The reaction was run with 2 equiv. of Int-C8 and 3.5 equiv. of Cs₂CO₃. The crude organic extracts were purified first, by normal phase flash column chromatography (12 g SiO₂ column) using an elution gradient of 0-6% MeOH (1% NH₄OH (aq.)) in DCM, and second, by prep-HPLC (Condition 10, Gradient b) to yield 5-[(2R,4S)-4-[6,7-dimethyl-4-[3- (trifluoromethyl)-1- bicyclo[1.1.1]pentanyl]pteridin-2- yl]tetrahydropyran-2-yl]-1- (ethylamino)pyridin-2-one (Example 181) as a solid. ESI-MS: [M + H]⁺ = 515.4. ¹H NMR (DMSO-d₆, 400 MHz): δ 7.69 (1H, |
| Example 181 | | d, J = 2.4 Hz), 7.47 (1H, dd, J = 9.3, 2.5 |
| | | Hz), 6.52 (1H, t, J = 6.1 Hz), 6.46 (1H, d, J = |
| | | 9.3 Hz), 4.36-4.39 (1H, m), 4.12-4.16 |
| | | (1H, m), 3.65-3.72 (1H, m), 3.27-3.27 (1H, |
| | | m), 2.95 (2H, p, J = 6.8 Hz), 2.72 (6H, s), |
| | | 2.61 (6H, s), 2.15-2.20 (1H, m), 1.98-2.03 |
| | | (1H, m), 1.90 (1H, td, J = 12.6, 4.6 Hz), |
| | | 1.77 (1H, q, J = 12.2 Hz), 0.99 (3H, t, J = |
| | | 7.2 Hz). ¹⁹F NMR (DMSO-d₆, 376 MHz): |
| | | δ −71.4 (3H, s). |

### Method 25: Synthesis of Example 180

### Example 180:5-[(2S,6R)-4-[6-(difluoromethyl)-7-methyl-4-[3-(trifluoromethyl)-1-bicyclo[1.1.1]pentanyl]pteridin-2-yl]-6-methyl-morpholin-2-yl]-1-methyl-pyridin-2-one

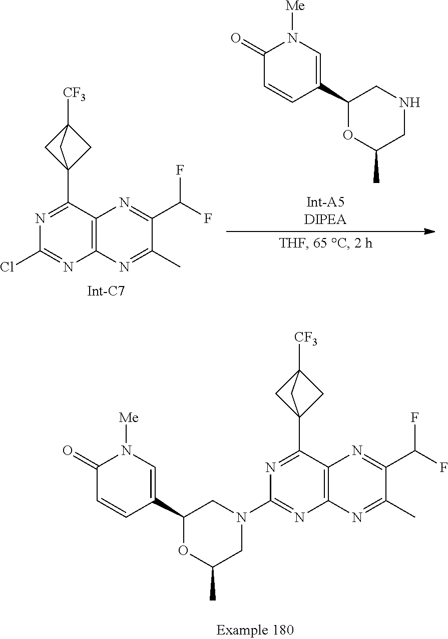 [SMILES: Cc1nc2nc(Cl)nc(C34CC(C(F)(F)F)(C3)C4)c2nc1C(F)F ; C[C@@H]1CNC[C@H](c2ccc(=O)n(C)c2)O1 ; Cc1nc2nc(N3C[C@@H](C)O[C@@H](c4ccc(=O)n(C)c4)C3)nc(C34CC(C(F)(F)F)(C3)C4)c2nc1C(F)F]

A MW vial was charged with 2-chloro-6-(difluoromethyl)-7-methyl-4-[3-(trifluoromethyl)-1-bicyclo[1.1.1]pentanyl]pteridine (Int-C7, 1 equiv., 70 mg, 0.146 mmol) and 1-methyl-5-[(2S,6R)-6-methylmorpholin-2-yl]pyridin-2-one (3)(1.05 equiv., 32 mg, 0.153 mmol) in anhydrous THF (1 mL). Hunig's base (3 equiv., 76 μL, 0.438 mmol) was added and the reaction mixture was stirred at 65° C. for 2 h. After 2 h, the reaction mixture was cooled down, quenched with water (20 mL) and diluted in EtOAc (25 mL). The aqueous phase was extracted with EtOAc (3×25 mL). The combined organic phases were washed with brine, dried over anhydrous Na₂SO₄, filtered and concentrated under reduced pressure. The resulting residue was purified by flash column chromatography (12 g SiO₂ column) using an elution gradient of 0-10% MeOH in DCM to yield 5-[(2S,6R)-4-[6-(difluoromethyl)-7-methyl-4-[3-(trifluoromethyl)-1-bicyclo[1.1.1]pentanyl]pteridin-2-yl]-6-methyl-morpholin-2-yl]-1-methyl-pyridin-2-one (Example 180, 59 mg, 0.11 mmol, 76% yield) was obtained as a solid. ESI-MS: [M+H]+: 537.4. 1H NMR (CDCl₃, 400 MHz): δ 7.44 (2H, m), 6.72 (1H, t, J=54.1 Hz), 6.69 (1H, m), 5.02 (2H, m), 4.38 (1H, d, J=10.5 Hz), 3.80 (1H, m), 3.60 (3H, s), 2.83-2.87 (5H, m), 2.59 (6H, s), 1.36 (3H, s). ¹⁹F NMR (CDCl₃, 376 MHz): δ −73.1 (3F, s), -114.3 (2F, d).

An analogous method was followed to obtain the following compounds.

| Structure | Starting Material | Characterization |
|---|---|---|
| 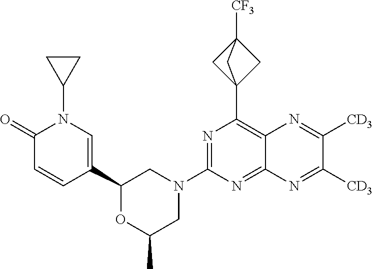 [SMILES: [2H]C([2H])([2H])c1nc2nc(N3C[C@@H](C)O[C@@H](c4ccc(=O)n(C5CC5)c4)C3)nc(C34CC(C(F)(F)F)(C3)C4)c2nc1C([2H])([2H])[2H]] Example 182 | 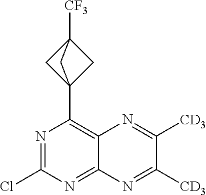 [SMILES: [2H]C([2H])([2H])c1nc2nc(Cl)nc(C34CC(C(F)(F)F)(C3)C4)c2nc1C([2H])([2H])[2H]] Int-C13 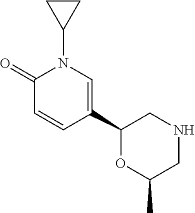 | Modification: The reaction was run at 70 °C for 1 h. The crude residue was purified by flash chromatography (12 g SiO₂ column) using an elution gradient of 50-100% EtOAc in hexanes to yield 5-[(2S,6R)-4-[6,7-bis(methyl-d₃)-4-[3- (trifluoromethyl)-1- bicyclo[1.1.1]pentan-1-yl]pteridin-2- yl]-6-methyl-morpholin-2-yl]-1- cyclopropylpyridin-2(1H)-one (Example 182, 28 mg, 53 μmol, 62% yield) as a solid. ¹H NMR (CDCl₃, 400 MHz): δ 7.40 (s, 2H), 6.60 (dt, J = 9.5, 1.6 Hz, 1H), 4.90-5.00 (m, 2H), 4.32- 4.36 (m, 1H), 3.75-3.81 (m, 1H), 3.31- 3.37 (m, 1H), 2.84-2.91 (m, 1H), 2.74- 2.80 (m, 1H), 2.59 (s, 6H), 1.32-1.34 (m, 3H), 1.13-1.18 (m, H), 0.86-0.92 (m, 2H). ¹⁹F NMR (CDCl₃, 376 MHz): δ −73.1 (s, 3F). ESI-M: [M + H]⁺ = 533.3. |
| | Int-C12 | |
| 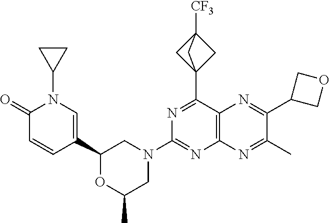 [SMILES: Cc1nc2nc(N3C[C@@H](C)O[C@@H](c4ccc(=O)n(C5CC5)c4)C3)nc(C34CC(C(F)(F)F)(C3)C4)c2nc1C1COC1] Example 183 | 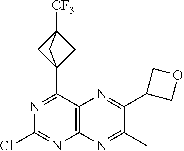 Int-C14 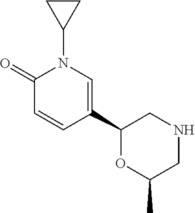 | Modification: The crude residue was purified, first, by normal phase flash chromatography (12 g SiO₂ column) using an elution gradient of 0-10% MeOH in DCM, and second, by reverse phase flash column chromatography (Condition 8, Gradient a) to yield 1-cyclopropyl-5- ((2S,6R)-6-methyl-4-(7-methyl-6- (oxetan-3-yl)-4-(3- (trifluoromethyl)bicyclo[1.1.1]pentan- 1-yl)pteridin-2-yl)morpholin-2- yl)pyridin-2(1H)-one (Example 183, 41 mg, 0.072 mmol, 23% yield) as a solid. ESI-MS: [M + H]⁺ = 569.2. ¹H NMR (CDCl₃, 400 MHz): δ 7.42-7.43 (1H, m), 6.65 (1H, d, J = 9.4 Hz), 5.11 (2H, s), 5.09 (2H, s), 4.91-5.04 (2H, m), 4.64 (1H, p, J = 7.7 Hz), 4.36 (1H, dd, J = 10.8, 2.6 Hz), 3.77-3.82 (1H, m), 3.33-3.37 (1H, m), 2.92 (1H, dd, J = 13.3, 10.8 Hz), 2.81 (1H, dd, J = 13.4, 10.6 Hz), 2.62 (6H, s), 2.55 (3H, s), 1.34 (3H, d, J = 6.2 Hz), 1.17 (2H, d, J = 7.2 Hz), 0.91 (2H, d, J = 5.0 Hz). ¹⁹F NMR (CDCl₃, 376 MHz): δ −73.0 (3F, |
| | Int-C12 | s). |
| 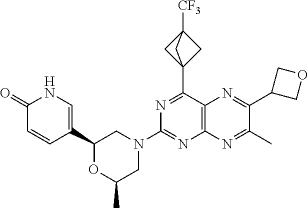 [SMILES: Cc1nc2nc(N3C[C@@H](C)O[C@@H](c4ccc(=O)[nH]c4)C3)nc(C34CC(C(F)(F)F)(C3)C4)c2nc1C1COC1] Example 195 | 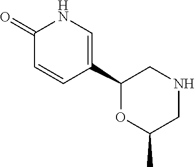 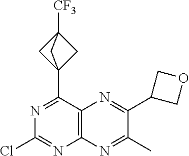 Int-C14 | The method yielded 5-[(2S,6R)-6- methyl-4-[7-methyl-6-(oxetan-3-yl)-4- [3-(trifluoromethyl)-1- bicyclo[1.1.1]pentanyl]pteridin-2- yl]morpholin-2-yl]-1H-pyridin-2-one (Example 195, 13 mg, 0.024 mmol, 21% yield) as solid. ESI-MS: [M + H]⁺ = 529.3. ¹H NMR (DMSO-d₆, 400 MHz): δ 11.61 (1H, s), 7.52 (1H, dd, J = 9.5, 2.6 Hz), 7.41 (1H, d, J = 2.5 Hz), 6.35 (1H, d, J = 9.4 Hz), 4.95 (2H, dd, J = 8.4, 5.5 Hz), 4.90 (2H, t, J = 6.2 Hz), 4.66-4.74 (2H, m), 4.36-4.39 (1H, m), 3.67-3.73 (1H, m), 3.29-3.29 (1H, m), 2.92-3.01 (1H, m), 2.72-2.80 (1H, m), 2.58 (6H, s), 2.45 (3H, s), 1.22 (3H, d, J = 6.3 Hz). ¹⁹F NMR (DMSO-d₆, 376 MHz): δ −71.4 (3F, s). |

### Method 26: Synthesis of Example 190

### Example 190:5-[1-[4-(2,4-difluorophenyl)-6,7-dimethyl-pteridin-2-yl]-4,4-difluoro-3-piperidyl]-1-methyl-pyridin-2-one

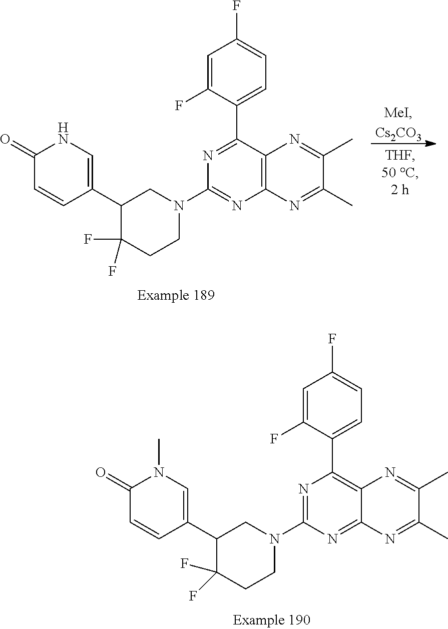 [SMILES: Cc1nc2nc(N3CCC(F)(F)C(c4ccc(=O)[nH]c4)C3)nc(-c3ccc(F)cc3F)c2nc1C ; Cc1nc2nc(N3CCC(F)(F)C(c4ccc(=O)n(C)c4)C3)nc(-c3ccc(F)cc3F)c2nc1C]

Cs₂CO₃ (3 equiv, 61 mg, 0.19 mmol) was added to a solution of 5-[1-[4-(2,4-difluorophenyl)-6,7-dimethyl-pteridin-2-yl]-4,4-difluoro-3-piperidyl]-1H-pyridin-2-one (Compound 189, 1 equiv, 30 mg, 0.062 mmol) and methyl iodide (2 equiv, 7.7 μL, 0.12 mmol) in THF (2.0 mL). The mixture was stirred at 50° C. for 2 hours, and then washed with water and extracted three times with DCM. The combined organic layers were washed with brine, dried over Na₂SO₄, filtered, and concentrated under reduced pressure. The crude was purified by reverse phase chromatography (40% to 80% MeCN in water containing 0.5% formic acid) to afford 5-[1-[4-(2,4-difluorophenyl)-6,7-dimethyl-pteridin-2-yl]-4,4-difluoro-3-piperidyl]-1-methyl-pyridin-2-one (Compound 190, 14 mg, 0.028 mmol, 45% yield) as a solid. ESI-MS: [M+H]⁺=499.3. ¹H NMR (CDCl₃, 400 MHz): δ 7.70 (1H, q, J=7.6 Hz), 7.35 (1H, d, J=9.5 Hz), 7.26 (1H, s), 7.04 (1H, t, J=8.2 Hz), 6.97 (1H, t, J=9.6 Hz), 6.57 (1H, d, J=9.4 Hz), 5.19 (2H, br s), 3.55 (3H, s), 3.40 (2H, q, J=12.5 Hz), 2.93 (1H, br d, J=27.7 Hz), 2.71 (3H, s), 2.60 (3H, s), 2.28 (1H, br s), 1.99-2.15 (1H, m). ¹⁹F NMR (CDCl₃, 376 MHz): δ −95.9 (1F, d, J=247.7 Hz), -106.5 (1F, s), -106.7 (1F, s), -113.7 (1F, d, J=246.7 Hz).

An analogous procedure was followed to obtain the following compounds.

| Structure | Intermediate | Characterization |
|---|---|---|
| 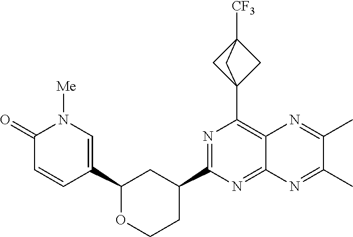 | 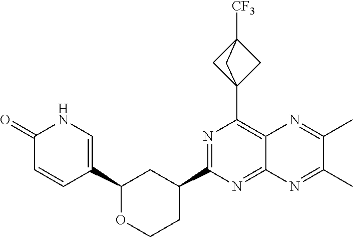 [SMILES: Cc1nc2nc([C@H]3CCO[C@@H](c4ccc(=O)[nH]c4)C3)nc(C34CC(C(F)(F)F)(C3)C4)c2nc1C] | The N-methylation of of Compound 197 was run with 3 equiv. of methyl iodide and 2 equiv. of K₂CO₃. The crude mixture was purified by silica gel flash-column chromatography using an elution gradient of 30%-65% acetone in hexanes to yield 1- methyl-5-((2R,4S)-4-(6-methyl-4-(3- (trifluoromethyl)bicyclo[1.1.1]pentan-1- yl)pteridin-2-yl)tetrahydro-2H-pyran-2- yl)pyridin-2(1H)-one (Example 198, 0.86 g, 1.83 mmol, 96% yield) as a foam. ESI- MS: [M + H]⁺ = 472.4. ¹H NMR (CDCl₃, 400 MHz): δ 9.01 (1H, s), 7.40-7.43 (2H, m), 6.61 (1H, d, J = 9.7 Hz), 4.27-4.34 (2H, m), 3.76-3.83 (1H, m), 3.56 (3H, s), 3.44- |
| Example 198 | Example 197 | 3.52 (1H, m), 2.82 (3H, s), 2.66 (6H, s), |
| | | 2.31 (1H, d, J = 13.2 Hz), 2.10-2.18 (2H, |
| | | m), 1.96-2.05 (1H, m). ¹⁹F NMR (CDCl₃, |
| | | 376 MHz): δ −73.1 (3F, s). |
| 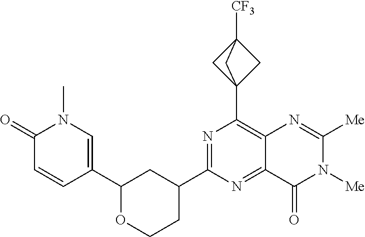 [SMILES: Cc1nc2c(C34CC(C(F)(F)F)(C3)C4)nc(C3CCOC(c4ccc(=O)n(C)c4)C3)nc2c(=O)n1C] | 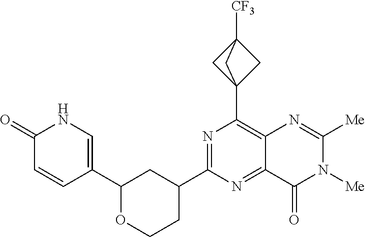 [SMILES: Cc1nc2c(C34CC(C(F)(F)F)(C3)C4)nc(C3CCOC(c4ccc(=O)[nH]c4)C3)nc2c(=O)n1C] | Modifications: The reaction was run with 3 equiv. of Cs₂CO₃ at 45 °C for 1 h. The reaction mixture was cooled down to r.t., concentrated under reduced pressure, and purified by flash chromatography (4 g SiO₂) using an elution gradient of 0-50% EtOAc:EtOH (3:1) in hexanes to yield 2,3- dimethyl-6-[rac-(2R,4S)-2-(1-methyl-6- oxo-3-pyridyl)tetrahydropyran-4-yl]-8-[3- (trifluoromethyl)-1- bicyclo[1.1.1]pentanyl]pyrimido[5,4- d]pyrimidin-4-one (Example 217, 3.0 mg, 6.0 umol, 32% yield) as a solid. ESI-MS: [M + H]⁺ = 502.3. ¹H NMR (CDCl₃, 400 MHz): δ 7.38-7.36 (m, 2H), 6.58-6.55 (m, |
| Example 217 | Example 216 | 1H), 4.30-4.24 (m, 2H), 3.75 (td, J = 11.9, |
| | | 2.4 Hz, 1H), 3.66 (s, 3H), 3.55 (s, 3H), 3.48 |
| | | (tt, J = 12.0, 3.9 Hz, 1H), 2.66 (s, 3H), 2.58 |
| | | (s, 6H), 2.20 (d, J = 13.3 Hz, 1H), 2.17- |
| | | 2.06 (m, 1H), 2.01 (d, J = 13.3 Hz, 1H), |
| | | 1.91 (dd, J = 24.7, 11.9 Hz, 1H). ¹⁹F NMR |
| | | (CDCl₃, 376 MHz): δ −73.0 (s, 3F). |
| 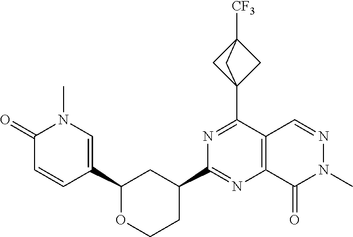 [SMILES: Cn1cc([C@H]2C[C@@H](c3nc(C45CC(C(F)(F)F)(C4)C5)c4cnn(C)c(=O)c4n3)CCO2)ccc1=O] | 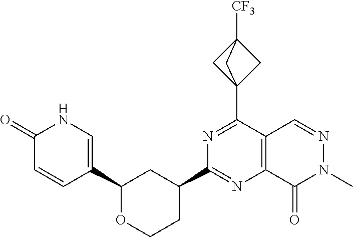 | The crude mixture was purified by silica gel flash-column chromatography using an elution gradient of 1-10% MeOH in DCM to yield 7-methyl-2-((2R,4S)-2-(1-methyl- 6-oxo-1,6-dihydropyridin-3-yl)tetrahydro- 2H-pyran-4-yl)-4-(3- (trifluoromethyl)bicyclo[1.1.1]pentan-1- yl)pyrimido[4,5-d]pyridazin-8(7H)-one (Example 228, 14 mg, 0.027 mmol, 86% yield) as a solid. ESI-MS: [M + H]⁺ = 488.3. ¹H NMR (400 MHz, CDCl₃): δ 8.43 (s, 1H), 7.35-7.33 (m, 2H), 6.56 (dd, J = 6.9, 3.4 Hz, 1H), 4.29-4.24 (m, 2H), 3.90 (s, 3H), 3.74 (td, J = 11.9, 2.4 Hz, 1H), 3.58-3.51 (m, 4H), |
| Example 228 | Example 227 | 2.61 (s, 6H), 2.21 (d, J = 13.3 Hz, 1H), |
| | | 2.13-2.00 (m, 2H), 1.90 (dd, J = 24.7, 11.9 |
| | | Hz, 1H). ¹⁹F NMR (376 MHz, CDCl₃-d): |
| | | δ −73.0 (s, 3F). |
| 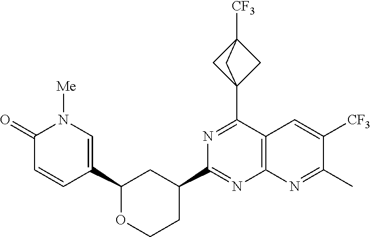 [SMILES: Cc1nc2nc([C@H]3CCO[C@@H](c4ccc(=O)n(C)c4)C3)nc(C34CC(C(F)(F)F)(C3)C4)c2cc1C(F)(F)F] | 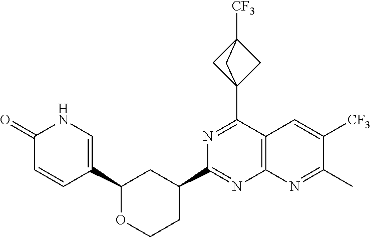 | Modifications: the reaction was run at r.t. for 1 h, and purified by normal phase flash column chromatography (4 g SiO₂ column) (0-10% MeOH in DCM), followed by reverse phase flash column chromatography (10-90% MeCN in 0.1% formic acid (aq.)) to yield 1-methyl-5- [(2R,4S)-4-[7-methyl-6-(trifluoromethyl)- 4-[3-(trifluoromethyl)-1- bicyclo[1.1.1]pentanyl]pyrido[2,3- d]pyrimidin-2-yl]tetrahydropyran-2- yl]pyridin-2-one (Example 326, 6.0 mg, 0.011 mmol, 29% yield) as a solid. ESI- MS: [M + H]⁺ = 539.2. ¹H NMR (CDCl₃, 400 MHz): δ 8.78 (1H, s), 7.41 (2H, d, J = |
| Example 326 | Int-E10 | 4.6 Hz), 6.61-6.66 (1H, m), 4.28-4.36 (2H, |
| | | m), 3.75-3.82 (1H, m), 3.57 (3H, s), 3.44- |
| | | 3.52 (1H, m), 2.96 (3H, s), 2.66 (6H, s), |
| | | 2.29-2.35 (1H, m), 2.02-2.20 (3H, m). ¹⁹F |
| | | NMR (CDCl₃, 376 MHz): δ −62.6 (3F, s), |
| | | −73.1 (3F, s). |
| 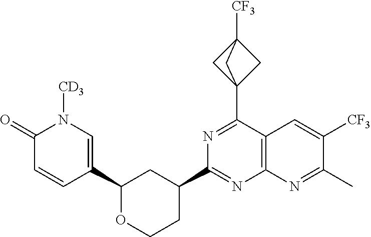 [SMILES: [2H]C([2H])([2H])n1cc([C@H]2C[C@@H](c3nc(C45CC(C(F)(F)F)(C4)C5)c4cc(C(F)(F)F)c(C)nc4n3)CCO2)ccc1=O] | 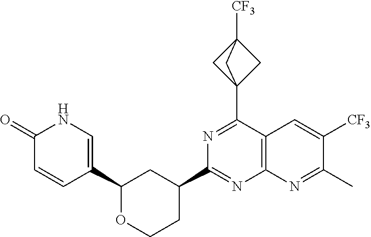 [SMILES: Cc1nc2nc([C@H]3CCO[C@@H](c4ccc(=O)[nH]c4)C3)nc(C34CC(C(F)(F)F)(C3)C4)c2cc1C(F)(F)F] | Modifications: The reaction was performed with 2 equiv. Cs2CO3 and 3 equiv. CD₃I over 2.5 h. The crude mixture was purified by SiO₂ column chromatography (20-50% acetone in hexanes) to yield 1-(methyl-d₃)-5- ((2R,4S)-4-(7-methyl-6-(trifluoromethyl)- 4-(3- (trifluoromethyl)bicyclo[1.1.1]pentan-1- yl)pyrido[2,3-d]pyrimidin-2- yl)tetrahydro-2H-pyran-2-yl)pyridin- 2(1H)-one (Example 334, 56 mg, 0.101 mmol, 81% yield) as a solid. ESI-MS: [M + H]⁺ = 542.3. ¹H NMR (CDCl₃, 400 MHz): δ 8.76 (s, 1H), 7.48 (d, J = 8.0 Hz, |
| Example 334 | Example 330 | 2H), 6.81 (d, J = 9.8 Hz, 1H), 4.37 (d, J = |
| | | 11.0 Hz, 1H), 4.31-4.28 (m, 1H), 3.79 (td, |
| | | J = 11.5, 3.1 Hz, 1H), 3.50-3.45 (m, 1H), |
| | | 2.95 (s, 3H), 2.64 (s, 6H), 2.32 (d, J = 13.0 |
| | | Hz, 1H), 2.17-1.97 (m, 4H). ¹⁹F NMR |
| | | (CDCl₃, 376 MHz): δ −62.5 (s, 3F), −73.0 |
| | | (s, 3F). |
| 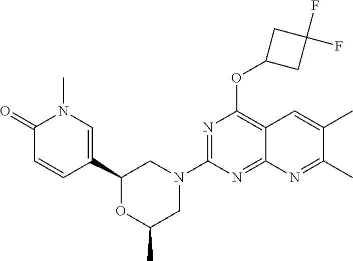 [SMILES: Cc1cc2c(OC3CC(F)(F)C3)nc(N3C[C@@H](C)O[C@@H](c4ccc(=O)n(C)c4)C3)nc2nc1C] | 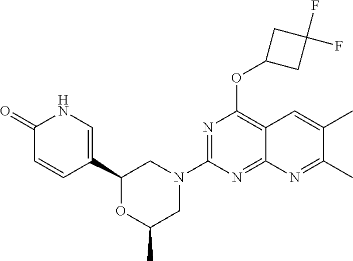 [SMILES: Cc1cc2c(OC3CC(F)(F)C3)nc(N3C[C@@H](C)O[C@@H](c4ccc(=O)[nH]c4)C3)nc2nc1C] | Modifications: The reaction was run with 1.05 equiv. MeI, and the crude was purified by flash chromatography (Isco RediSep ® column 4 g, using a gradient eluction of 0-10% MeOH in DCM) to obtain 5-((2S,6R)-4(4-(3,3- difluorocyclobutoxy)-6,7- dimethylpyrido[2,3-d]pyrimidin-2-yl)-6- methylmorpholin-2-yl)-1-methylpyridin- 2(1H)-one (Example 340, 14 mg, 29.7 μmol, 54%) as a solid. ¹H NMR (CDCl₃, 400 MHz): δ 7.91 (s, 1H), 7.40-7.43 (m, 2H), 6.60-6.62 (d, J = 10.1Hz, 1H), 5.29 (s, 3H), 4.31-4.34 (m, 1H), 3.72-3.80 (m, 1H), 3.57 (s, 3H), 3.11-3.21 (m, 2H), 2.95-2.79 (m, 3H), 2.78-2.69 (dd, J = 12.2, 10.1Hz, |
| Example 340 | Int-E14 | 1H), 2.64 (s, 3H), 2.36 (s, 3H), 1.32 (d, J = |
| | | 6.2 Hz, 3H). ¹⁹F NMR (CDCl₃, 376 MHz): |
| | | δ −84.6 (s, 1F), −96.7 (s, IF). ESI-MS: |
| | | [M + H]⁺ = 472.4. |
| 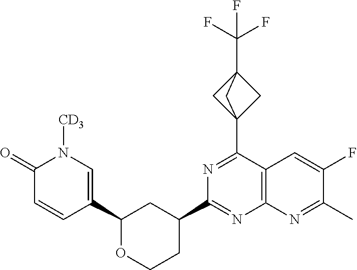 [SMILES: [2H]C([2H])([2H])n1cc([C@H]2C[C@@H](c3nc(C45CC(C(F)(F)F)(C4)C5)c4cc(F)c(C)nc4n3)CCO2)ccc1=O] | CD₃I 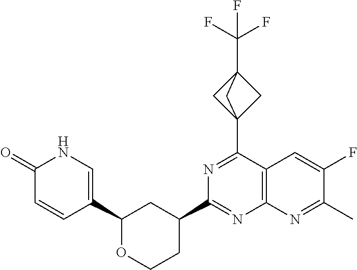 [SMILES: Cc1nc2nc([C@H]3CCO[C@@H](c4ccc(=O)[nH]c4)C3)nc(C34CC(C(F)(F)F)(C3)C4)c2cc1F] | Modificiations: The reaction was run with 1.5 equiv. CD₃I at r.t. overnight. The residue was purified by chromatography (4 g SiO₂) using 0-15% DCM in MeOH elution gradient to yield 5-((2R,4S)-4-(6- fluoro-7-methyl-4-(3- (trifluoromethyl)bicyclo[1.1.1]pentan-1- yl)pyrido[2,3-d]pyrimidin-2- yl)tetrahydro-2H-pyran-2-yl)-1-(methyl- d₃)pyridin-2(1H)-one (Example 301, 16 mg, 0.03 mmol, 44% yield) as a solid. ¹H NMR (CDCl₃, 400 MHz): δ 8.03 (1H, d, J = 8.5 Hz), 7.37-7.40 (2H, m), 6.54-6.57 (1H, m), 4.24-4.31 (2H, m), 3.73-3.80 (1H, m), 3.40- 3.46 (1H, m), 2.79 (3H, d, J = 2.9 Hz), 2.61 (7H, s), 2.29 (1H, d, J = 13.4 Hz), 1.96-2.14 (4H, m). ¹⁹F NMR (CDCl₃, 376 MHz): δ −73.2 (3F, s), −120.2 (1F, s). ESI- MS: [M + H]⁺ = 492.3. |
| Example 301 | Int-E24 | |
| 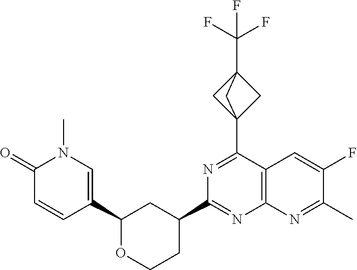 [SMILES: Cc1nc2nc([C@H]3CCO[C@@H](c4ccc(=O)n(C)c4)C3)nc(C34CC(C(F)(F)F)(C3)C4)c2cc1F] | 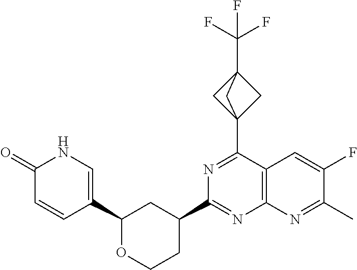 | Modificiations: The reaction was run with 1.5 equiv. MeI at r.t. overnight to afford 5- ((2R,4S)-4-(6-fluoro-7-methyl-4-(3- (trifluoromethyl)bicyclo[1.1.1]pentan-1- yl)pyrido[2,3-d]pyrimidin-2- yl)tetrahydro-2H-pyran-2-yl)-1- methylpyridin-2(1H)-one (Example 307). ¹H NMR (CDCl₃, 400 MHz): δ 8.03 (1H, d, J = 8.5 Hz), 7.37-7.39 (2H, m), 6.56 (1H, d, J = 9.9 Hz), 4.29 (2H, t, J = 13.1 Hz), 3.77 (1H, t, J = 11.5 Hz), 3.54 (3H, s), 3.42 (1H, br s), 2.79 (3H, d, J = 2.8 Hz), 2.61 (6H, s), 2.29 (1H, d, J = 13.4 Hz), 2.12 (2H, s), 1.95-2.04 (2H, m). ¹⁹F NMR (CDCl₃, 376 MHz): δ −73.2 (3F, s), −120.2 (1F, s). ESI-MS: [M + H]⁺ = 489.3. |
| Example 307 | Int-E24 | |
| 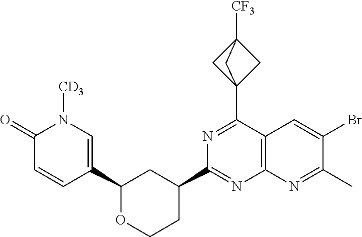 [SMILES: [2H]C([2H])([2H])n1cc([C@H]2C[C@@H](c3nc(C45CC(C(F)(F)F)(C4)C5)c4cc(Br)c(C)nc4n3)CCO2)ccc1=O] Example 303 | CD₃I 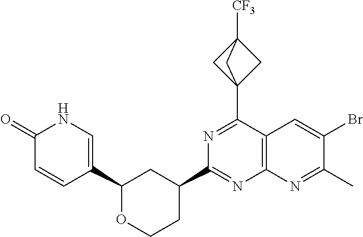 [SMILES: Cc1nc2nc([C@H]3CCO[C@@H](c4ccc(=O)[nH]c4)C3)nc(C34CC(C(F)(F)F)(C3)C4)c2cc1Br] | Modificiations: The reaction was run with 3 equiv. CD₃I at 60 °C for 1 h. The crude material was purified by flash chromatography (0-10% MeOH in DCM elution gradient) to afford 5-((2R,4S)-4-(6- bromo-7-methyl-4-(3- (trifluoromethyl)bicyclo[1.1.1]pentan-1- yl)pyrido[2,3-d]pyrimidin-2- yl)tetrahydro-2H-pyran-2-yl)-1-(methyl- d₃)pyridin-2(1H)-one (Example 303, 365 mg, 78% yield) as a solid. ESI-MS: [M + H]⁺ = 552.1. ¹H NMR (CDCl₃, 400 MHz): δ 8.62 (1H, s), 7.36-7.38 (2H, m), 6.55 (1H, d, J = 9.5 Hz), 4.29 (2H, t, J = 12.4 Hz), 3.73-3.80 (1H, m), 3.37-3.45 (1H, m), 2.92 (3H, s), 2.62 (6H, s), 2.27- 2.31 (1H, m), 2.06-2.12 (2H, m), 1.95-2.04 |
| | Int-E31 | (1H, m). ¹⁹F NMR (CDCl₃, 376 MHz): δ −73.1. |
| 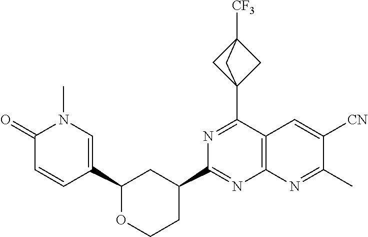 [SMILES: Cc1nc2nc([C@H]3CCO[C@@H](c4ccc(=O)n(C)c4)C3)nc(C34CC(C(F)(F)F)(C3)C4)c2cc1C#N] | 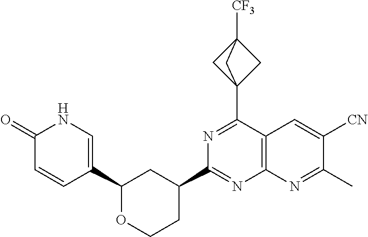 [SMILES: Cc1nc2nc([C@H]3CCO[C@@H](c4ccc(=O)[nH]c4)C3)nc(C34CC(C(F)(F)F)(C3)C4)c2cc1C#N] | Modification: The reaction was run with 3 equiv. of Cs₂CO₃ and purified by reverse flash chromatography using 30-85% MeCN in water (0.1% of formic acid) to afford 5-((2R,4S)-4-(6,7-dimethyl-4-(3- (trifluoromethyl)bicyclo[1.1.1]pentan-1- yl)pteridin-2-yl)tetrahydro-2H-pyran-2- yl)-1-methylpyridin-2(1H)-one (Example 25, 522 mg, 85% yield) as a solid. ¹H NMR (CDCl₃, 400 MHz): δ 7.37-7.40 (2H, m), 6.54-6.57 (1H, m), 4.23-4.31 (2H, m), 3.76 (1H, td, J = 11.5, 3.3 Hz), 3.53 (3H, s), 3.38-3.46 (1H, m), 2.78 (3H, s), 2.74 (3H, s), 2.61 (6H, s), 2.28 (1H, d, J = 13.3 Hz), 2.05-2.13 (2H, m), 1.94-2.03 |
| Example 25 | Example 34 | (1H, m). ESI-MS: [M + H]⁺ = 486.4. |
| 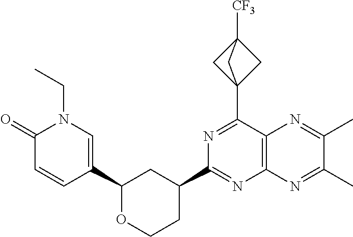 [SMILES: CCn1cc([C@H]2C[C@@H](c3nc(C45CC(C(F)(F)F)(C4)C5)c4nc(C)c(C)nc4n3)CCO2)ccc1=O] Example 80 | iodoethane 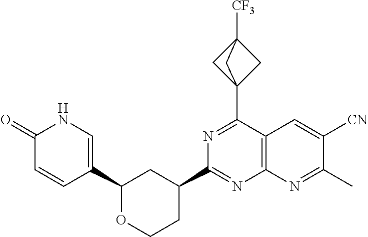 | Modification: The reaction was run with 3 equiv. of Cs₂CO₃ to afford 5-[(2R,4S)-4- [6,7-dimethyl-4-[3-(trifluoromethyl)-1- bicyclo[1.1.1]pentanyl]pteridin-2- yl]tetrahydropyran-2-yl]-1-ethyl-pyridin- 2-one (Example 80). ¹H NMR (CDCl₃, 400 MHz): δ 7.39-7.36 (2H, m), 6.56-6.54 (1H, m), 4.34-4.28 (2H, m), 4.01-3.96 (2H, m), 3.82-3.76 (1H, m), 3.48-3.42 (1H, m), 2.81 (3H, s), 2.77 (3H, s), 2.65 (6H, s), 2.31 (1H, d, J = 13.3 Hz), 2.14-2.11 (2H, m), 2.03 (1H, q, J = 12.3 Hz), 1.36 (3H, t, J = 7.2 Hz). ¹⁹F NMR (CDCl₃, 376 MHz): δ −73.1 (3F, s). ESI-MS: [M + H]⁺ = 500.4. |
| | Example 34 | |
| 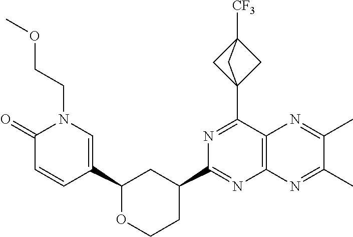 [SMILES: COCCn1cc([C@H]2C[C@@H](c3nc(C45CC(C(F)(F)F)(C4)C5)c4nc(C)c(C)nc4n3)CCO2)ccc1=O] Example 82 | 1-bromo-2-methoxyethane 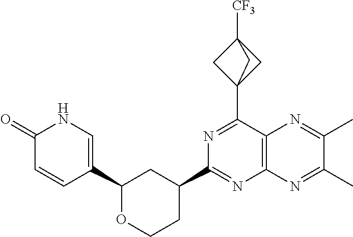 | Modification: The reaction was run with 3 equiv. of Cs₂CO₃ to afford 5-[(2R,4S)-4- [6,7-dimethyl-4-[3-(trifluoromethyl)-1- bicyclo[1.1.1]pentanyl]pteridin-2- yl]tetrahydropyran-2-yl]-1-(2- methoxyethyl)pyridin-2-one (Example 82). ¹H NMR (CDCl₃, 400 MHz): δ 7.44- 7.39 (2H, m), 6.56 (1H, d, J = 9.3 Hz), 4.33-4.4.27 (2H, m), 4.10 (2H, t, J = 5.1 Hz), 3.81-3.75 (1H, m), 3.67 (2H, t, J = 5.1 Hz), 3.48-3.41 (1H, m), 3.32 (3H, s), 2.81 (3H, s), 2.77 (3H, s), 2.65 (6H, s), 2.29 (1H, d, J = 13.4 Hz), 2.15-2.11 (2H, m), 2.07-1.98 (1H, m). ¹⁹F NMR (CDCl₃, 376 MHz): δ −73.1 (3F, s). ESI-MS: [M + H]⁺ = 530.4. |
| | Example 34 | |
| 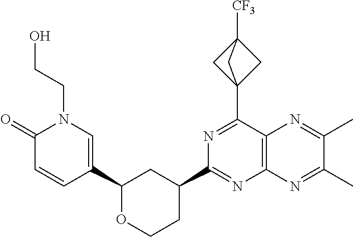 [SMILES: Cc1nc2nc([C@H]3CCO[C@@H](c4ccc(=O)n(CCO)c4)C3)nc(C34CC(C(F)(F)F)(C3)C4)c2nc1C] Example 84 | 2-bromoethanol 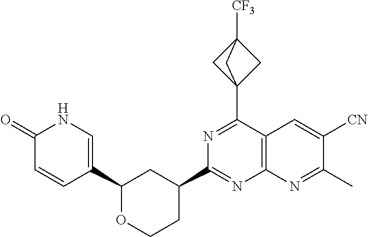 [SMILES: Cc1nc2nc([C@H]3CCO[C@@H](c4ccc(=O)[nH]c4)C3)nc(C34CC(C(F)(F)F)(C3)C4)c2cc1C#N] | Modification: The reaction was run with 3 equiv. of Cs₂CO₃ to afford 5-[(2R,4S)-4- [6,7-dimethyl-4-[3-(trifluoromethyl)-1- bicyclo[1.1.1]pentanyl]pteridin-2- yl]tetrahydropyran-2-yl]-1-(2- hydroxyethyl)pyridin-2-one (Example 84). ¹H NMR (CDCl₃, 400 MHz): δ 7.45 (1H, dd, J = 9.4, 2.4 Hz), 7.42-7.38 (1H, m), 6.61 (1H, d, J = 9.3 Hz), 4.34-4.25 (2H, m), 4.15 (2H, t, J = 4.7 Hz), 3.97-3.95 (2H, m), 3.82-3.75 (1H, m), 3.46-3.39 (2H, m), 2.81 (3H, s), 2.77 (3H, s), 2.65 (6H, s), 2.34-2.30 (1H, m), 2.15-2.08 (2H, m), 2.05-1.96 (1H, m). ¹⁹F NMR (CDCl₃, 376 MHz): δ −73.1 (3F, s). ESI-MS: [M + H]⁺ = 516.3. |
| | Example 34 | |
| 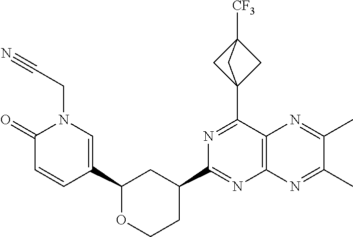 [SMILES: Cc1nc2nc([C@H]3CCO[C@@H](c4ccc(=O)n(CC#N)c4)C3)nc(C34CC(C(F)(F)F)(C3)C4)c2nc1C] Example 86 | 2-bromoacetonitrile 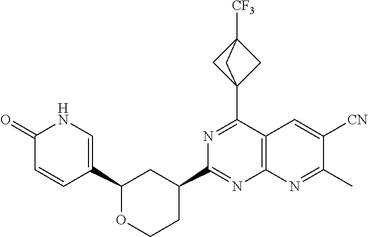 | Modification: The reaction was run with 3 equiv. of Cs₂CO₃ to afford 2-[5-[(2R,4S)- 4-[6,7-dimethyl-4-[3-(trifluoromethyl)-1- bicyclo[1.1.1]pentanyl]pteridin-2- yl]tetrahydropyran-2-yl]-2-oxo-1- pyridyl]acetonitrile (Example 86). ¹H NMR (CDCl₃, 400 MHz): δ 7.46-7.43 (2H, m), 6.63-6.60 (1H, m), 4.84 (2H, s), 4.35-4.26 (2H, m), 3.81-3.75 (1H, m), 3.48-3.40 (1H, m), 2.81 (3H, s), 2.77 (3H, s), 2.64-2.63 (6H, m), 2.34 (1H, d, J = 13.4 Hz), 2.19-2.07 (2H, m), 1.98 (1H, q, J = 12.4 Hz). ¹⁹F NMR (CDCl₃, 376 MHz): δ −73.1 (3F, s). ESI-MS: [M + H]⁺ = 511.3. |
| | Example 34 | |
| 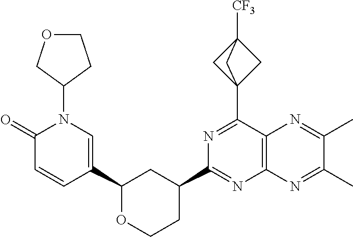 [SMILES: Cc1nc2nc([C@H]3CCO[C@@H](c4ccc(=O)n(C5CCOC5)c4)C3)nc(C34CC(C(F)(F)F)(C3)C4)c2nc1C] Example 96 | 3-iodotetrahydrofuran 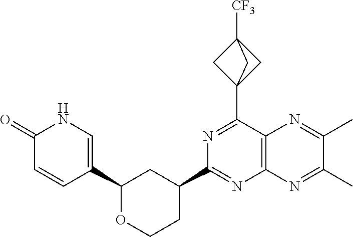 | Modification: The reaction was run with 3 equiv. of Cs₂CO₃ to afford 5-[(2R,4S)-4- [6,7-dimethyl-4-[3-(trifluoromethyl)-1- bicyclo[1.1.1]pentanyl]pteridin-2- yl]tetrahydropyran-2-y1]-1H-pyridin-2- one (Example 96). ¹H NMR (CDCl₃, 400 MHz): δ 7.50 (1H, s), 7.40 (1H, dt, J = 9.4, 3.0 Hz), 6.55 (1H, d, J = 9.5 Hz), 5.68-5.63 (1H, m), 4.32-4.25 (2H, m), 4.21-4.13 (1H, m), 4.01 (1H, t, J = 8.9 Hz), 3.93-3.88 (1H, m), 3.84-3.73 (2H, m), 3.46-3.39 (1H, m), 2.80 (3H, s), 2.76 (3H, s), 2.64-2.62 (6H, m), 2.60-2.52 (1H, m), 2.29-2.24 (1H, m), 2.16-2.10 (2H, m), 2.01-1.92 (2H, m). ¹⁹F NMR (CDCl₃, 376 MHz): δ −73.1 (3F, s). ESI-MS: [M + H]⁺ = 542.4. |
| | Example 34 | |
| 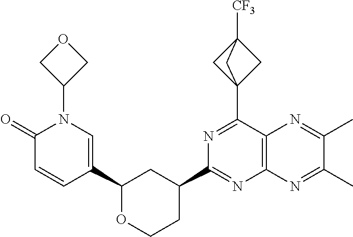 [SMILES: Cc1nc2nc([C@H]3CCO[C@@H](c4ccc(=O)n(C5COC5)c4)C3)nc(C34CC(C(F)(F)F)(C3)C4)c2nc1C] Example 107 | 3-iodooxetane 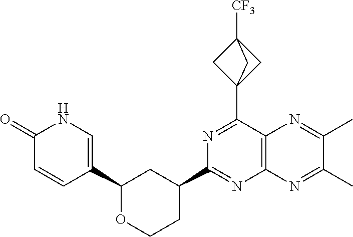 | Modification: The reaction was run with 3 equiv. of Cs₂CO₃ to afford 5-[(2R,4S)-4- [6,7-dimethyl-4-[3-(trifluoromethyl)-1- bicyclo[1.1.1]pentanyl]pteridin-2- yl]tetrahydropyran-2-yl]-1-(oxetan-3- yl)pyridin-2-one (Example 107). ¹H NMR (CDCl₃, 400 MHz): δ 7.65 (1H, d, J = 2.4 Hz), 7.40 (1H, dd, J = 9.4, 2.5 Hz), 6.52 (1H, d, J = 9.4 Hz), 5.80 (1H, p, J = 6.9 Hz), 5.08 (2H, td, J = 7.4, 1.8 Hz), 4.78 (2H, td, J = 6.8, 2.8 Hz), 4.37 (1H, d, J = 11.3 Hz), 4.27-4.31 (1H, m), 3.75-3.82 (1H, m), 3.41-3.49 (1H, m), 2.79 (3H, s), 2.76 (3H, s), 2.63 (6H, s), 2.32 (1H, d, J = 13.4 Hz), 2.09-2.15 (2H, m), 1.97-2.07 (1H, m). ¹⁹F NMR (CDCl₃, 376 MHz): δ −73.1 (3F, s). ESI-MS: [M + H]⁺ = 528.3. |
| | Example 34 | |
| 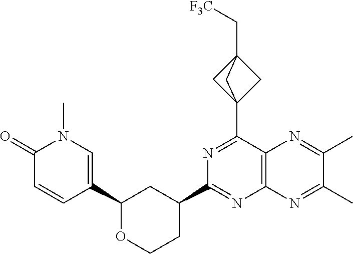 [SMILES: Cc1nc2nc([C@H]3CCO[C@@H](c4ccc(=O)n(C)c4)C3)nc(C34CC(CC(F)(F)F)(C3)C4)c2nc1C] | 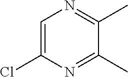 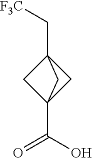 [SMILES: O=C(O)C12CC(CC(F)(F)F)(C1)C2] | Starting from the building blocks of 5- chloro-2,3-dimethyl-pyrazine and 3- (2,2,2-trifluoroethyl)bicyclo[1.1.1] pentane-1-carboxylic acid 5-[(2R,4S)-4-[6,7-dimethyl-4-[3-(2,2,2- trifluoroethyl)-1- bicyclo[1.1.1]pentanyl]pteridin-2- yl]tetrahydro-2H-pyran-2-yl]-1-methyl- pyridin-2(1H)-one (Example 54) was obtained using the same synthetic route as Example 80. ¹H NMR (CDCl₃, 400 MHz): δ 7.39-7.42 (2H, m), 6.58 (1H, dd, J = 8.8, 1.4 Hz), 4.25-4.32 (2H, m), 3.74-3.81 (1H, m), 3.55 (3H, s), 3.39-3.47 (1H, m), 2.79 (3H, s), 2.76 (3H, s), 2.50 (6H, s), 2.45 (2H, q, J = 11.1 Hz), 2.28-2.32 (1H, m), |
| Example 54 | | 2.11-2.15 (2H, m), 1.98-2.10 (2H, m). ¹⁹F |
| | | NMR (CDCl₃, 376 MHz): δ −64.2 (3F, t, J = |
| | | 11 Hz). ESI-MS: [M + H]⁺ = 500.4 |
| 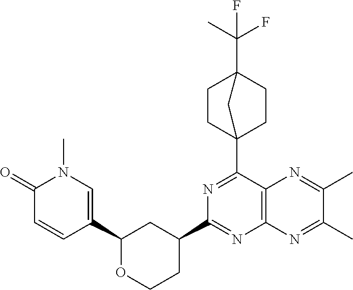 [SMILES: Cc1nc2nc([C@H]3CCO[C@@H](c4ccc(=O)n(C)c4)C3)nc(C34CCC(C(C)(F)F)(CC3)C4)c2nc1C] | 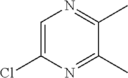 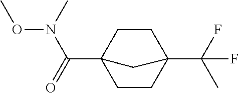 Int-F12 | Starting from the building blocks of 5- chloro-2,3-dimethyl-pyrazine and Int-F12, 5-((2R,4S)-4-(4-(4-(1,1- difluoroethyl)bicyclo[2.2.1]heptan-1-yl)- 6,7-dimethylpteridin-2-yl)tetrahydro-2H- pyran-2-yl)-1-methylpyridin-2(1H)-one (Example 60) was obtained using the same synthetic route as Example 80. ¹H NMR (CDCl₃, 400 MHz): δ 7.37-7.40 (2H, m), 6.54-6.57 (1H, m), 4.30 (1H, d, J = 11.8 Hz), 4.24-4.27 (1H, m), 3.71-3.80 (1H, m), 3.54 (3H, s), 3.36-3.44 (1H, m), 2.78 (3H, s), 2.74 (3H, s), 2.44 (2H, t, J = 9.8 Hz), 2.32 (1H, d, J = 13.6 Hz), 2.26 (2H, s), 2.02-2.15 (7H, m), 1.93-1.99 (2H, m), 1.64-1.67 (3H, m). ¹⁹F NMR (CDCl₃, 376 MHz): δ −96.4 (2F, s). ESI-MS: [M + H]⁺ = 510.4. |
| Example 60 | | |
| 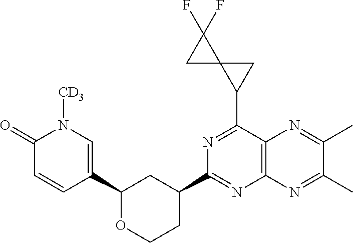 [SMILES: [2H]C([2H])([2H])n1cc([C@H]2C[C@@H](c3nc(C4CC45CC5(F)F)c4nc(C)c(C)nc4n3)CCO2)ccc1=O] | 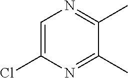 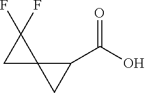 | Starting from the building blocks of 5- chloro-2,3-dimethyl-pyrazine and 4,4- difluorospiro[2.2]pentane-1-carboxylic acid, 5-((2R,4S)-4-(4-(4,4- difluorospiro[2.2]pentan-1-yl)-6,7- dimethylpteridin-2-yl)tetrahydro-2H- pyran-2-yl)-1-(methyl-d3)pyridin-2(1H)- one (Example 92) was obtained using the same synthetic route as Example 80. ¹H NMR (CDCl₃, 400 MHz): δ 7.36-7.39 (2H, m), 6.57 (1H, d, J = 9.5 Hz), 4.24-4.31 (2H, m), 4.16 (1H, t, J = 6.6 Hz), 3.73-3.80 (1H, m), 3.34-3.41 (1H, m), 2.81 (3H, s), 2.78 (3H, s), 2.29 (1H, t, J = 12.4 Hz), 1.88-2.13 (6H, m), 1.52 (1H, t, J = 9.2 Hz). |
| Example 92 | | ¹⁹F NMR (CDCl₃, 376 MHz): δ −133.22 |
| | | (1F, dd, J = 152.0, 8.9 Hz), −134.21 (1F, dt, |
| | | J = 152.2, 8.5 Hz). ESI-MS: [M + H]⁺ = |
| | | 457.4. |

### Method 27: Synthesis of Example 191

### Example 191:5-((2S,6R)-4-(5-(2,4-difluorophenyl)-2,3-dimethylpyrido[3,4-b]pyrazin-7-yl)-6-methylmorpholin-2-yl)-1-methylpyridin-2 (1H)-one

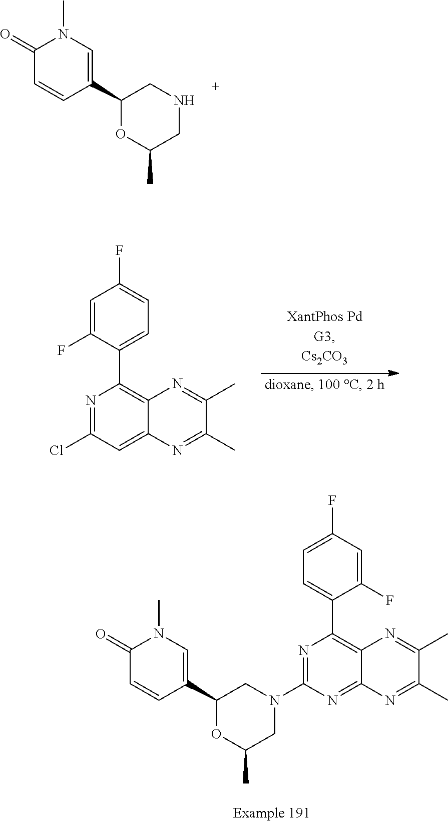 [SMILES: C[C@@H]1CNC[C@H](c2ccc(=O)n(C)c2)O1 ; Cc1nc2cc(Cl)nc(-c3ccc(F)cc3F)c2nc1C ; Cc1nc2nc(N3C[C@@H](C)O[C@@H](c4ccc(=O)n(C)c4)C3)nc(-c3ccc(F)cc3F)c2nc1C]

From 1-methyl-5-((2S,6R)-6-methylmorpholin-2-yl)pyridin-2 (1H)-one and 7-chloro-5-(2,4-difluorophenyl)-2,3-dimethylpyrido[3,4-b]pyrazine, a coupling procedure analogous to Method 5 from WO 2022/236272 was utilized to obtain 5-((2S,6R)-4-(5-(2,4-difluorophenyl)-2,3-dimethylpyrido[3,4-b]pyrazin-7-yl)-6-methylmorpholin-2-yl)-1-methylpyridin-2 (1H)-one (Example 191). ¹H NMR (CD₃OD, 400 MHz): δ 7.77 (1H, d, J=2.4 Hz), 7.68 (1H, dd, J=9.3, 2.5 Hz), 7.61 (1H, s), 7.53-7.59 (1H, m), 7.16 (2H, t, J=8.7 Hz), 7.00 (1H, s), 6.57 (1H, d, J=9.3 Hz), 4.52-4.55 (1H, m), 4.43 (1H, d, J=12.8 Hz), 4.29 (1H, d, J=12.6 Hz), 3.90-3.92 (1H, m), 3.59 (3H, s), 2.80 (1H, t, J=11.7 Hz), 2.64-2.71 (4H, m), 2.35 (3H, s), 1.33 (3H, d, J=6.2 Hz). ¹⁹F NMR (CD₃OD, 376 MHz): δ −110.68 (1F, m), -112.2 (1F, m). ESI-MS: [M+H]⁺=478.4.

### Method 28: Synthesis of Example 196

### Example 196:5-((2S,6R)-4-(5-(2,4-difluorophenyl)-2,3-dimethyl-1,6-naphthyridin-7-yl)-6-methylmorpholin-2-yl)-1-methylpyridin-2 (1H)-one

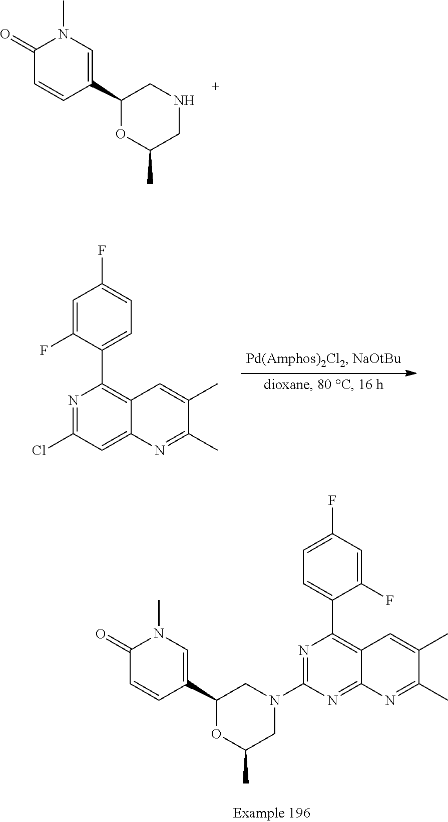 [SMILES: C[C@@H]1CNC[C@H](c2ccc(=O)n(C)c2)O1 ; Cc1cc2c(-c3ccc(F)cc3F)nc(Cl)cc2nc1C ; Cc1cc2c(-c3ccc(F)cc3F)nc(N3C[C@@H](C)O[C@@H](c4ccc(=O)n(C)c4)C3)nc2nc1C]

From 1-methyl-5-((2S,6R)-6-methylmorpholin-2-yl)pyridin-2 (1H)-one and 7-chloro-5-(2,4-difluorophenyl)-2,3-dimethyl-1,6-naphthyridine, a coupling procedure analogous to Method 4 from WO 2022/236272 was utilized to obtain 5-((2S,6R)-4-(5-(2,4-difluorophenyl)-2,3-dimethyl-1,6-naphthyridin-7-yl)-6-methylmorpholin-2-yl)-1-methylpyridin-2 (1H)-one (Example 196). ESI-MS: [M+H]⁺=477.4. ¹H NMR (CD₃OD, 400 MHz): δ 7.77 (1H, s), 7.68 (1H, dd, J=9.3, 2.4 Hz), 7.62 (1H, q, J=7.6 Hz), 7.02-7.10 (2H, m), 7.01 (1H, s), 6.57 (1H, d, J=9.3 Hz), 4.54 (1H, d, J=10.8 Hz), 4.47 (1H, d, J=13.0 Hz), 4.34 (1H, d, J=12.7 Hz), 3.91 (1H, s), 3.59 (3H, s), 2.83 (1H, t, J=11.7 Hz), 2.67-2.74 (4H, m), 2.57 (3H, s), 1.33 (3H, d, J=6.2 Hz). ¹⁹F NMR (CD₃OD, 376 MHz): δ −110.2 (1F, m), -111.5 (1F, m).

### Method 29: Synthesis of Example 194

### Example 194:5-(4-(5-(2,4-difluorophenyl)-2,3-dimethylpyrido[3,4-b]pyrazin-7-yl)tetrahydro-2H-pyran-2-yl)-1-methylpyridin-2 (1H)-one

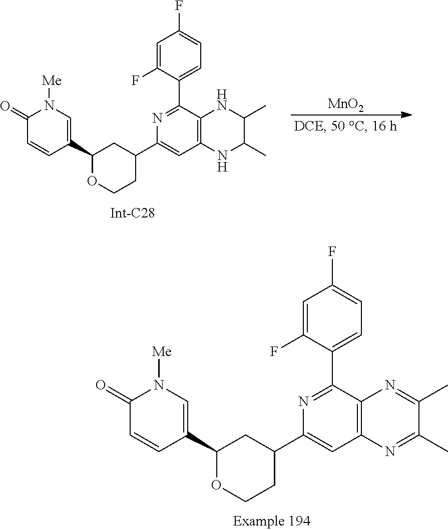 [SMILES: CC1Nc2cc(C3CCO[C@@H](c4ccc(=O)n(C)c4)C3)nc(-c3ccc(F)cc3F)c2NC1C ; "MnO2" ; Cc1nc2cc(C3CCO[C@@H](c4ccc(=O)n(C)c4)C3)nc(-c3ccc(F)cc3F)c2nc1C]

To a flame-dried glass vial under argon equipped with a Teflon-coated magnetic stirring bar, was added 5-(4-(5-(2,4-difluorophenyl)-2,3-dimethyl-1,2,3,4-tetrahydropyrido[3,4-b]pyrazin-7-yl)tetrahydro-2H-pyran-2-yl)-1-methylpyridin-2 (1H)-one (Int-C28, 1 equiv., 40 mg, 0.09 mmol), MnO₂ (20 equiv., 149 mg, 1.72 mmol), and anhydrous DCE (0.8 mL). The vial was sealed and purged under argon, and the reaction mixture was stirred at 50° C. overnight. The reaction solution was then filtered over a pad of Celite and washed with 9:1 DCM:MeOH (100 mL). The filtrate was concentrated under reduced pressure, and the crude mixture was first purified by flash column chromatography on an ISCO CombiFlash system (4 g SiO₂ column, using an elution gradient from 35% to 100% EtOAc in DCM) and then by preparative HPLC (Condition 10, Gradient c) to afford 5-(4-(5-(2,4-difluorophenyl)-2,3-dimethylpyrido[3,4-b]pyrazin-7-yl)tetrahydro-2H-pyran-2-yl)-1-methylpyridin-2 (1H)-one (Example 194, 3 mg, 6.5 μmol, 8% yield) as a solid. ¹H NMR (CDCl₃, 400 MHz): δ 7.70 (s, 1H), 7.61-7.67 (m, 1H), 7.37-7.40 (m, 2H), 7.05 (td, J=8.31, 2.42 Hz, 1H), 6.96 (td, J=9.43, 2.5 Hz, 1H), 6.59 (d, J=9.2 Hz 1H), 4.28-4.35 (m, 2H), 3.80 (td, J=11.7, 2.8 Hz, 1H), 3.55 (s, 3H), 3.32-3.38 (m, 1H), 2.76 (s, 3H), 2.68 (s, 3H), 2.30-2.34 (m, 1H), 2.00-2.13 (m, 2H). ¹⁹F NMR (CDCl₃, 376 MHz): δ −107.9 (s, 1F), -108.9 (s, 1F). ESI-MS: [M+H]⁺=463.4.

### Method 31: Synthesis of Example 201

### Example 201:5-((2R,4S)-4-(6,7-bis(methyl-d₃)-4-(3-(trifluoromethyl) bicyclo[1.1.1]pentan-1-yl) pteridin-2-yl)tetrahydro-2H-pyran-2-yl)-1-cyclopropylpyridin-2 (1H)-one

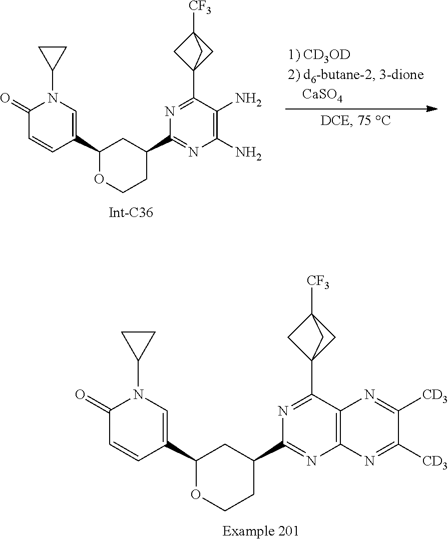 [SMILES: Nc1nc([C@H]2CCO[C@@H](c3ccc(=O)n(C4CC4)c3)C2)nc(C23CC(C(F)(F)F)(C2)C3)c1N ; [2H]C([2H])([2H])c1nc2nc([C@H]3CCO[C@@H](c4ccc(=O)n(C5CC5)c4)C3)nc(C34CC(C(F)(F)F)(C3)C4)c2nc1C([2H])([2H])[2H]]

1-Cyclopropyl-5-((2R,4S)-4-(4,5-diamino-6-(3-(trifluoromethyl) bicyclo[1.1.1]pentan-1-yl)pyrimidin-2-yl)tetrahydro-2H-pyran-2-yl)pyridin-2 (1H)-one (Int-C36, 1 equiv., 72 mg, 0.16 mmol) was dissolved in CD₃OD and concentrated under reduced pressure. This initial process was repeated three times. Anhydrous DCE (2.0 mL), anhydrous CaSO₄ (10 equiv., 212 mg, 1.6 mmol) and 1,1,1,4,4,4-hexadeuteriobutane-2,3-dione (1.5 equiv., 0.022 mL, 0.234 mmol) were added and the reaction vessel was sealed and stirred at 75° C. for 18 h. The reaction mixture was cooled to r.t. and concentrated under reduced pressure onto Celite (4.0 g). The crude mixture was purified by silica gel flash column chromatography using an elution gradient of 1-10% MeOH in DCM to yield 5-((2R,4S)-4-(6,7-bis(methyl-d3)-4-(3-(trifluoromethyl) bicyclo[1.1.1]pentan-1-yl) pteridin-2-yl)tetrahydro-2H-pyran-2-yl)-1-cyclopropylpyridin-2 (1H)-one (Example 201, 73 mg, 0.14 mmol, 89% yield) as a solid. ESI-MS: [M+H]⁺=518.4. ¹H NMR (CD₃OD, 400 MHz): δ 7.59-7.62 (2H, m), 6.53 (1H, d, J=9.1 Hz), 4.43 (1H, d, J=11.3 Hz), 4.25 (1H, dt, J=11.5, 3.1 Hz), 3.78-3.84 (1H, m), 3.42-3.49 (1H, m), 3.32-3.35 (1H, m), 2.66 (6H, s), 2.24 (1H, d, J=13.1 Hz), 2.05-2.09 (2H, m), 1.96 (1H, dd, J=24.4, 12.3 Hz), 1.10-1.15 (2H, m), 0.90-0.94 (2H, m). ¹⁹F NMR (CD₃OD, 376 MHz): δ −74.8 (3F, s).

An analogous procedure was followed to obtain the following compounds.

| Structure | Intermediate | Characterization |
|---|---|---|
| 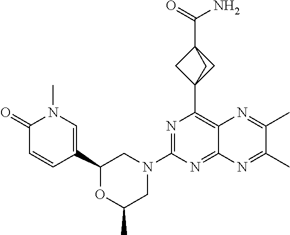 [SMILES: Cc1nc2nc(N3C[C@@H](C)O[C@@H](c4ccc(=O)n(C)c4)C3)nc(C34CC(C(N)=O)(C3)C4)c2nc1C] | 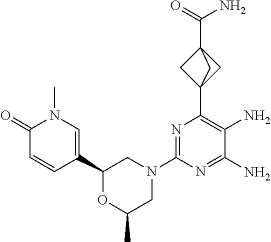 | Modifications: Int-C51 was reacted with 2,3-butadione at 80 °C for 16 h. The crude was purified first by normal phase chromatography (50- 100% EtOAc in hexanes) and then by reverse phase chromatography (15-85% ACN in water containing 0.1% FA) to afford 3-[6,7- dimethyl-2-[(2R,6S)-2-methyl-6- (1-methyl-6-oxo-3- pyridyl)morpholin-4-yl]pteridin-4- yl]bicyclo[1.1.1]pentane-1- carboxamide (Example 204, 14 mg, 0.029 mmol, 13% yield) as a solid. ESI-MS: [M + H]⁺ = 476.4 ¹H |
| Example 204 | Int-C51 | NMR (CDCl₃, 400 MHz) δ 7.40 (s, |
| | | 2H), 6.59 (d, J = 9.6 Hz, 1H), 5.63 |
| | | (d, J = 12.1 Hz, 2H), 4.97 (d, J = |
| | | 46.0 Hz, 2H), 4.35 (dd, J = 10.7, 2.3 |
| | | Hz, 1H), 3.80 (s, 1H), 3.56 (s, 3H), |
| | | 2.80 (ddd, J = 37.8, 13.2, 10.9 Hz, |
| | | 2H), 2.66 (s, 3H), 2.64 (s, 6H), 2.61 |
| | | (s, 3H), 1.32 (d, J = 6.2 Hz, 3H). |

### Method 32: Synthesis of Example 202

### Example 202:5-((2R,4S)-4-(8-(dimethylamino)-7-methyl-6-(3-(trifluoromethyl) bicyclo[1.1.1]pentan-1-yl)-7H-purin-2-yl)tetrahydro-2H-pyran-2-yl)-1-methylpyridin-2 (1H)-one

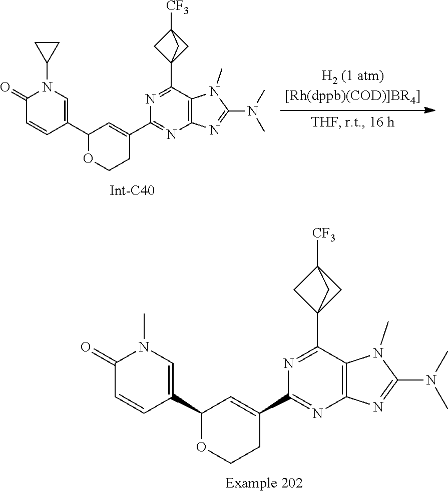

To a stirring solution of (5-(4-(8-(dimethylamino)-7-methyl-6-(3-(trifluoromethyl) bicyclo[1.1.1]pentan-1-yl)-7H-purin-2-yl)-5,6-dihydro-2H-pyran-2-yl)-1-methylpyridin-2 (1H)-one (Int-C40, 1 equiv., 190 mg, 0.39 mmol) in anhydrous THF (4.0 mL) was added [Rh(dppb)(COD)]BF₄ (0.24 equiv., 65 mg, 0.09 mmol). The resulting solution was stirred at r.t. under an atmosphere of H₂ for 16 h. The reaction was purged under argon and concentrated under reduced pressure onto Celite (5.0 g). The crude mixture was purified by silica gel flash column chromatography using an elution gradient of 40-100% acetone in hexanes and then by preparative HPLC (Condition 16, Gradient a) to yield 5-((2R,4S)-4-(8-(dimethylamino)-7-methyl-6-(3-(trifluoromethyl) bicyclo[1.1.1]pentan-1-yl)-7H-purin-2-yl)tetrahydro-2H-pyran-2-yl)-1-methylpyridin-2 (1H)-one (Example 202) as a solid. ESI-MS: [M+H]⁺=503.4. ¹H NMR (CDCl₃, 400 MHz): δ 7.35-7.39 (2H, m), 6.55 (1H, d, J=9.2 Hz), 4.28 (1H, d, J=11.4 Hz), 4.22 (1H, d, J=11.5 Hz), 3.71 (3H, s), 3.53 (3H, s), 3.17 (6H, s), 2.51 (6H, s), 2.21 (1H, d, J=13.2 Hz), 2.01-2.05 (2H, m), 1.92 (1H, dd, J=24.5, 12.2 Hz). ¹⁹F NMR (CDCl₃, 376 MHz): δ −72.9 (3F, s).

An analogous procedure was followed to obtain the following compounds.

| Structure | Intermediate | Characterization |
|---|---|---|
| 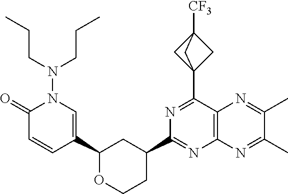 [SMILES: CCCN(CCC)n1cc([C@H]2C[C@@H](c3nc(C45CC(C(F)(F)F)(C4)C5)c4nc(C)c(C)nc4n3)CCO2)ccc1=O] | 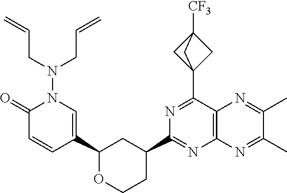 [SMILES: C=CCN(CC=C)n1cc([C@H]2C[C@@H](c3nc(C45CC(C(F)(F)F)(C4)C5)c4nc(C)c(C)nc4n3)CCO2)ccc1=O] | Modifications: The reaction was run at 55 °C for 16 h, concentrated under reduced pressure, and purified by flash chromatography (4 g SiO₂) using an elution gradient of 0-45% EtOAc:EtOH (3:1) in hexanes to yield 5-[(2R,4S)-4-[6,7-dimethyl-4-[3- (trifluoromethyl)-1- bicyclo[1.1.1]pentanyl]pteridin-2- yl]tetrahydropyran-2-y1]-1- (dipropylamino)pyridin-2-one (Example 211, 1.0 mg, 17.5 umol, 6% |
| Example 211 | Example 206 | yield) as a solid. |
| | | ESI-MS: [M + H]⁺ = 571.4. ¹H NMR |
| | | (400 MHz, CD₃OD): δ 7.59-7.55 (m, |
| | | 2H), 6.51 (d, J = 9.1 Hz, 1H), 4.42- |
| | | 4.39 (m, 1H), 4.25-4.22 (m, 1H), 3.83- |
| | | 3.76 (m, 1H), 3.49-3.42 (m, 3H), 2.97- |
| | | 2.90 (m, 2H), 2.78-2.76 (m, 6H), 2.64 |
| | | (s, 6H), 2.22 (d, J = 13.0 Hz, 1H), 2.07- |
| | | 2.02 (m, 2H), 1.93 (dd, J = 24.7, 11.9 |
| | | Hz, 1H), 1.39-1.26 (m, 4H), 0.90 (td, J = |
| | | 7.4, 4.2 Hz, 6H). ¹⁹F NMR (376 |
| | | MHz, CD₃OD): δ −74.7 (s, 3F). |
| 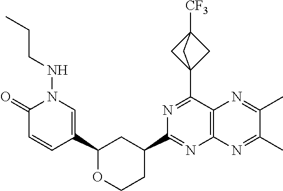 [SMILES: CCCNn1cc([C@H]2C[C@@H](c3nc(C45CC(C(F)(F)F)(C4)C5)c4nc(C)c(C)nc4n3)CCO2)ccc1=O] | 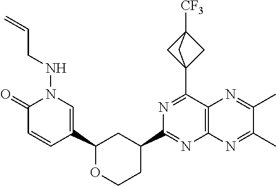 [SMILES: C=CCNn1cc([C@H]2C[C@@H](c3nc(C45CC(C(F)(F)F)(C4)C5)c4nc(C)c(C)nc4n3)CCO2)ccc1=O] | The reaction crude was purified by flash chromatography (4 g SiO₂) using an elution gradient of 0-100% EtOAc:EtOH (3:1) in hexanes to yield 5-[(2R,4S)-4-[6,7-dimethyl-4-[3- (trifluoromethyl)-1- bicyclo[1.1.1]pentanyl]pteridin-2- yl]tetrahydropyran-2-yl]-1- (propylamino)pyridin-2-one (Example 212, 7.0 mg, 13 μmol, 12% yield) as a solid. ESI-MS: [M + H]⁺ = 529.4. ¹H NMR |
| Example 212 | Example 207 | (400 MHz, CD₃OD): δ 7.79 (d, J = 2.3 |
| | | Hz, 1H), 7.61 (dd, J = 9.4, 2.5 Hz, 1H), |
| | | 6.63 (d, J = 9.4 Hz, 1H), 4.46 (d, J = |
| | | 9.6 Hz, 1H), 4.28-4.24 (m, 1H), 3.86- |
| | | 3.80 (m, 1H), 3.52-3.44 (m, 1H), 3.00 |
| | | (t, J = 7.2 Hz, 2H), 2.80 (s, 3H), 2.74- |
| | | 2.80 (3H), 2.67 (s, 6H), 2.28 (d, J = |
| | | 13.5 Hz, 1H), 2.11-2.06 (m, 2H), 1.97 |
| | | (dd, J = 24.8, 12.0 Hz, 1H), 1.56 (td, J = |
| | | 14.7, 7.4 Hz, 2H), 1.00 (t, J = 7.4 Hz, |
| | | 3H). ¹⁹F NMR (376 MHz, CD₃OD): δ |
| | | −74.7 (s, 3F). |
| 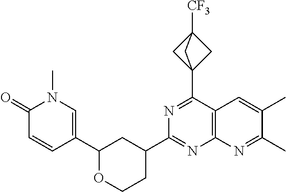 [SMILES: Cc1cc2c(C34CC(C(F)(F)F)(C3)C4)nc(C3CCOC(c4ccc(=O)n(C)c4)C3)nc2nc1C] | 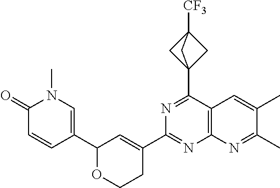 [SMILES: Cc1cc2c(C34CC(C(F)(F)F)(C3)C4)nc(C3=CC(c4ccc(=O)n(C)c4)OCC3)nc2nc1C] | The crude was purified by flash chromatography (Isco RediSep ® column 4 g) (1.5-3% MeOH in DCM) followed by prep-HPLC (Condition 10, Gradient f) to afford 5-(4-(6,7- dimethyl-4-(3- (trifluoromethyl)bicyclo[1.1.1]pentan- 1-yl)pyrido[2,3-d]pyrimidin-2- yl)tetrahydro-2H-pyran-2-yl)pyridin- 2(1H)-one (Example 325, 3.2 mg, 6.6 μmol, 8% yield) as a solid. ESI-MS: [M + H]⁺ = 485.3. ¹H NMR (400 MHz, |
| Example 325 | Int-A70 | CDCl₃): δ 8.18 (s, 1H), 7.38-7.40 (m, |
| | | 2H), 6.57 (d, J = 10.0 Hz, 1H), 4.29 (t, |
| | | J = 13.5 Hz, 2H), 3.78 (dd, J = 13.8, |
| | | 9.9 Hz, 1H), 3.55 (s, 3H), 3.41 (br s, |
| | | 1H), 2.76 (s, 3H), 2.62 (s, 6H), 2.51 (s, |
| | | 3H), 2.30 (d, J = 13.4 Hz, 1H), 2.01- |
| | | 2.17 (m, 3H). |
| 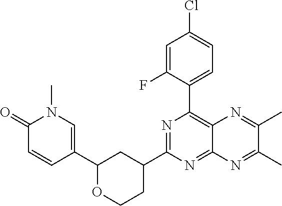 [SMILES: Cc1nc2nc(C3CCOC(c4ccc(=O)n(C)c4)C3)nc(-c3ccc(Cl)cc3F)c2nc1C] | 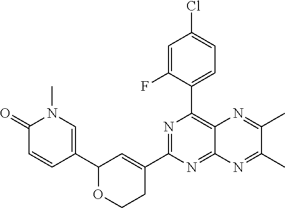 [SMILES: Cc1nc2nc(C3=CC(c4ccc(=O)n(C)c4)OCC3)nc(-c3ccc(Cl)cc3F)c2nc1C] | Modification: The reaction was run for 2 h at 50 °C and purified by pre- TLC (DCM/MeOH 10:1) followed by prep-HPLC to give 5-[4-[4-(4-chloro- 2-fluoro-phenyl)-6,7-dimethyl- pteridin-2-yl]tetrahydropyran-2-yl]-1- methyl-pyridin-2-one (Example 10, 1.2 mg, 0.0022 mmol, 1% yield) as a solid and 1-methyl-5-[rac-(2R,4R)-4- [4-(4-chloro-2-fluoro-phenyl)-6,7- dimethyl-pteridin-2- yl]tetrahydropyran-2-yl]pyridin-2-one (Example 9, 0.70 mg, 0.0013 mmol, |
| Example 10 | Int-E44 | 1% yield) as a solid. |
| 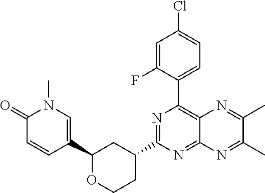 [SMILES: Cc1nc2nc([C@@H]3CCO[C@@H](c4ccc(=O)n(C)c4)C3)nc(-c3ccc(Cl)cc3F)c2nc1C] | | Example 10: LCMS: cis-mixture mixed with ~4% trans-mixture; [M + H]⁺ = 480.2. ¹H NMR (400 MHz, CDCl₃) δ 7.72 (t, J = 7.8 Hz, 1H), 7.45- 7.30 (m, 4H), 6.59 (d, J = 9.4 Hz, 1H), 4.59 (br d, J = 9.8 Hz, 1H), 4.03- 3.96 (m, 1H), 3.95-3.87 (m, 1H), 3.78 (br s, 1H), 3.56 (s, 3H), 2.87 (s, 3H), 2.82 (br d, J = 6.5 Hz, 1H), 2.76 (s, 3H), 2.63 (br d, J = 13.6 Hz, 1H), 2.31- 2.20 (m, 1H), 2.19-2.09 (m, 1H). ¹⁹F NMR (376 MHz, CDCl₃) δ −108.88 (s, |
| Example 9 | | 1F). |
| | | Example 9: LCMS: [M + H]⁺ = 480.2. |
| | | ¹H NMR (400 MHz, CDCl₃) δ 7.69 (t, |
| | | J = 7.8 Hz, 1H), 7.46-7.39 (m, 2H), |
| | | 7.36 (br d, J = 8.3 Hz, 1H), 7.30 (d, J = |
| | | 9.5 Hz, 1H), 6.60 (br d, J = 8.2 Hz, |
| | | 1H), 4.40-4.26 (m, 2H), 3.81 (dt, J = |
| | | 3.1, 11.3 Hz, 1H), 3.57 (s, 4H), 2.85 (s, |
| | | 3H), 2.74 (s, 3H), 2.38 (br d, J = 13.4 |
| | | Hz, 1H), 2.27-2.15 (m, 2H), 2.14- |
| | | 2.02 (m, 1H). ¹⁹F NMR (376 MHz, |
| | | CDCl₃) δ −108.49 (s, 1F). |

### Method 33: Synthesis of Example 205

### Example 205:3-[6,7-dimethyl-2-[(2R,6S)-2-methyl-6-(1-methyl-6-oxo-3-pyridyl) morpholin-4-yl]pteridin-4-yl]bicyclo[1.1.1]pentane-1-carbonitrile

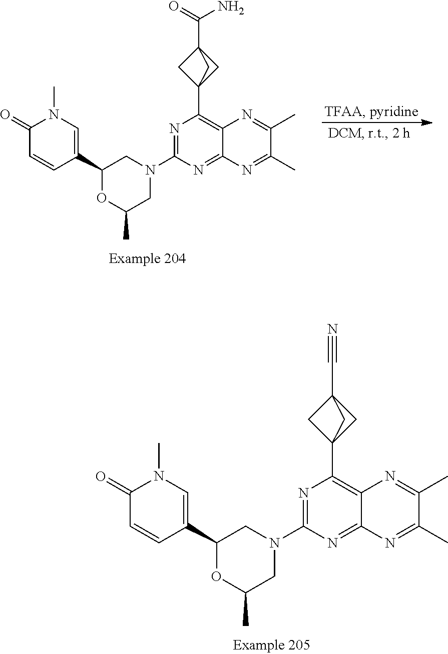 [SMILES: Cc1nc2nc(N3C[C@@H](C)O[C@@H](c4ccc(=O)n(C)c4)C3)nc(C34CC(C(N)=O)(C3)C4)c2nc1C ; Cc1nc2nc(N3C[C@@H](C)O[C@@H](c4ccc(=O)n(C)c4)C3)nc(C34CC(C#N)(C3)C4)c2nc1C]

TFAA (1 equiv., 0.015 mL, 0.11 mmol) was added dropwise to a solution of 3-[6,7-dimethyl-2-[(2R,6S)-2-methyl-6-(1-methyl-6-oxo-3-pyridyl) morpholin-4-yl]pteridin-4-yl]bicyclo[1.1.1]pentane-1-carboxamide (Example 204, 1 equiv., 50 mg, 0.11 mmol) and pyridine (2 equiv., 0.017 mL, 0.21 mmol) in DCM (1 mL), and the resulting mixture was stirred at room temperature for 1 hour. Another equivalent of TFAA (1 equiv., 0.015 mL, 0.11 mmol) was added, and the reaction was stirred for another 1 hour. The reaction mixture was concentrated under reduced pressure and purified by normal phase chromatography (50-100% EtOAc in hexanes) to afford 3-[6,7-dimethyl-2-[(2R,6S)-2-methyl-6-(1-methyl-6-oxo-3-pyridyl) morpholin-4-yl]pteridin-4-yl]bicyclo[1.1.1]pentane-1-carbonitrile (Example 205, 28 mg, 0.06 mmol, 58% yield) as a solid. ESI-MS: [M+H]⁺=458.4. ¹H NMR (CDCl₃, 400 MHz) δ 7.40 (d, J=6.9 Hz, 2H), 6.58 (d, J=9.8 Hz, 1H), 4.91 (d, J=29.7 Hz, 2H), 4.33 (dd, J=10.7, 2.5 Hz, 1H), 3.79-3.74 (m, 1H), 3.54 (s, 3H), 2.87-2.82 (m, 7H), 2.74 (dd, J=13.5, 10.7 Hz, 1H), 2.65 (s, 3H), 2.61 (s, 3H), 1.31 (d, J=5.9 Hz, 3H).

### Method 34: Synthesis of Example 206 and 207

### Example 206:1-(diallylamino)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one; and Example 207:1-(allylamino)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one

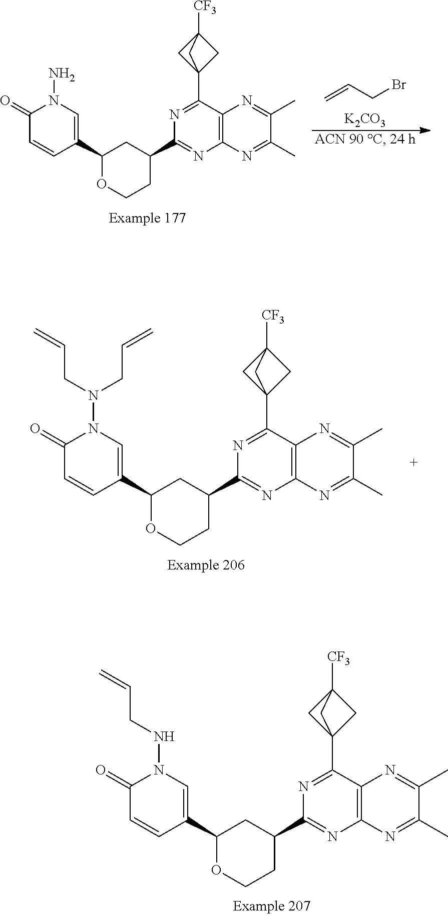

A 20 mL microwave vial was charged with 1-amino-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 177, 1 equiv., 395 mg, 0.81 mmol) and potassium carbonate (2.5 equiv., 281 mg, 2.0 mmol), MeCN (3.5 mL), and allyl bromide (2.2 equiv., 155 μL, 1.79 mmol) under argon atmosphere. The vial was sealed and stirred at 90° C. for 24 h. The reaction mixture was cooled down to r.t. and filtered through a fritted funnel. The filtrate was concentrated under reduced pressure and purified by flash chromatography (24 g SiO₂ column) using an elution gradient of 10-100% EtOAc:EtOH (3:1) in hexanes to yield two main products: 1-(diallylamino)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 206, 214 mg, 0.37 mmol, 46% yield) & 1-(allylamino)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 207, 60 mg, 0.11 mmol, 14% yield) as solids.

Example 206: ESI-MS: [M+H]⁺=567.4. ¹H NMR (DMSO-d₆, 400 MHz): δ 7.43-7.40 (m, 2H), 6.34 (d, J=9.8 Hz, 1H), 5.80 (s, 2H), 5.11 (d, J=16.5 Hz, 2H), 5.04 (d, J=9.4 Hz, 2H), 4.33 (d, J=9.8 Hz, 1H), 4.16-4.07 (m, 3H), 3.72-3.66 (m, 3H), 3.42-3.35 (m, 1H), 2.74 (d, J=2.1 Hz, 6H), 2.63 (s, 6H), 2.15-2.12 (m, 1H), 2.04-2.00 (m, 1H), 1.89 (ddd, J=25.4, 12.4, 4.5 Hz, 1H), 1.72 (q, J=12.1 Hz, 1H). ¹⁹F NMR (DMSO-d₆, 376 MHz): δ −71.3 (s, 3F).

Example 207: ESI-MS: [M+H]⁺=527.4. ¹H NMR (DMSO-d₆, 400 MHz): δ 7.64 (d, J=2.5 Hz, 1H), 7.43 (dd, J=9.4, 2.5 Hz, 1H), 6.74 (t, J=5.3 Hz, 1H), 6.43 (d, J=9.1 Hz, 1H), 5.87-5.77 (m, 1H), 5.08-5.06 (m, 1H), 5.03 (s, 1H), 4.35 (d, J=9.8 Hz, 1H), 4.11 (dd, J=11.2, 3.7 Hz, 1H), 3.67 (dd, J=11.8, 9.7 Hz, 1H), 3.57-3.53 (m, 2H), 3.35 (tt, J=11.9, 3.7 Hz, 1H), 2.70 (d, J=1.4 Hz, 6H), 2.59 (s, 6H), 2.14 (d, J=12.3 Hz, 1H), 1.99 (d, J=14.0 Hz, 1H), 1.92-1.84 (m, 1H), 1.73 (dd, J=24.6, 11.8 Hz, 1H). ¹⁹F NMR (DMSO-d6, 376 MHz): δ −71.3 (s, 3F).

An analogous procedure was followed to obtain the following compounds.

| Structure | Intermediate | Characterization |
|---|---|---|
| 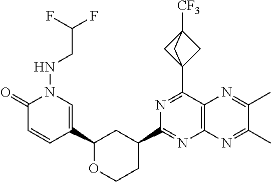 [SMILES: Cc1nc2nc([C@H]3CCO[C@@H](c4ccc(=O)n(NCC(F)F)c4)C3)nc(C34CC(C(F)(F)F)(C3)C4)c2nc1C] | 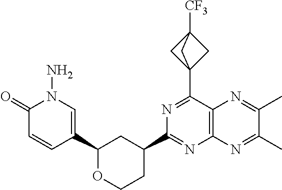 [SMILES: Cc1nc2nc([C@H]3CCO[C@@H](c4ccc(=O)n(N)c4)C3)nc(C34CC(C(F)(F)F)(C3)C4)c2nc1C] | Modifications: The reaction was run with 2,2-difluoroethyl trifluoromethanesulfonate for 16 h, and the crude was purified by flash chromatography (Isco RediSep ® column 12 g, using a gradient from 0- 100% AcOEt:EtOH (3:1) in hexanes). The material was further purified by reverse phase flash chromatography (Biotage column 6 g) using a gradient elution from 5-95% CH₃CN in water (0.1% of HCOOH). The selected |
| Example 214 | Example 177 | fractions were evaporated to yield 1- |
| | 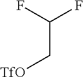 [SMILES: O=S(=O)(OCC(F)F)C(F)(F)F] | ((2,2-difluoroethyl)amino)-5-((2R,4S)- 4-(6,7-dimethyl-4-(3- (trifluoromethyl)bicyclo[1.1.1]pentan- 1-yl)pteridin-2-yl)tetrahydro-2H- pyran-2-yl)pyridin-2(1H)-one |
| | | (Example 214, 6.0 mg, 0.01 mmol, 9% |
| | | yield). ESI-MS: [M + H]⁺ = 551.3. ¹H |
| | | NMR (400 MHz, CD₃CN) δ 7.58 (d, J = |
| | | 2.5 Hz, 1H), 7.46 (dd, J = 9.5, 2.6 Hz, |
| | | 1H), 6.46 (d, J = 9.4 Hz, 1H), 6.19 (t, J = |
| | | 5.7 Hz, 1H), 5.99 (tt, J = 55.5, 4.1 Hz, |
| | | 1H), 4.34 (dd, J = 11.4, 1.8 Hz, 1H), |
| | | 4.19-4.15 (m, 1H), 3.73 (td, J = 11.8, |
| | | 2.7 Hz, 1H), 3.40-3.30 (m, 3H), 2.70 (d, |
| | | J = 3.0 Hz, 6H), 2.61 (s, 6H). Note: |
| | | some of the tetrahydropyran protons |
| | | are masked by the solvent peak. ¹⁹F |
| | | NMR (376 MHz, CD₃CN) δ −73.6 (s, |
| | | 3F), −122.9 (dt, J = 55.5, 15.6 Hz, 2F). |
| 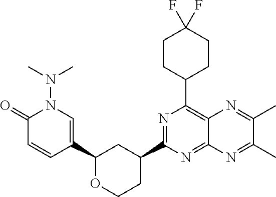 [SMILES: Cc1nc2nc([C@H]3CCO[C@@H](c4ccc(=O)n(N(C)C)c4)C3)nc(C3CCC(F)(F)CC3)c2nc1C] | 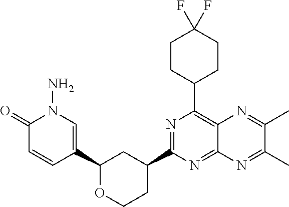 [SMILES: Cc1nc2nc([C@H]3CCO[C@@H](c4ccc(=O)n(N)c4)C3)nc(C3CCC(F)(F)CC3)c2nc1C] | Modifications: The reaction was run with 3 equiv. K₂CO₃ and 5 equiv. MeI in DMF solvent at 50 °C for 12 h. The crude was then purified by prep-HPLC (Condition 18, Gradient c) and lyophilized to afford 5-[(2R,4S)-4- [4-(4,4-difluorocyclohexyl)-6,7- dimethyl-pteridin-2- yl]tetrahydropyran-2-yl]-1- (dimethylamino)pyridin-2-one (Example 226, single enantiomer of known absolute configuration 61 mg, 0.113 mmol, 21% yield) as a solid. |
| Example 226 | Int-D43 | LCMS: [M + H]⁺ = 499.1. ¹H NMR (400 |
| | | MHz, CDCl₃) δ 2.18 (br s, 10H) 2.30- |
| | | 2.35 (m, 2H) 2.78 (s, 3H) 2.82 (s, 3H) |
| | | 3.03 (s, 6H) 3.44 (tt, J = 11.8, 3.9 Hz, |
| | | 1H) 3.74-3.86 (m, 1H) 4.12-4.22 (m, |
| | | 1H) 4.26-4.36 (m, 2H) 6.54 (d, J = 9.4 |
| | | Hz, 1H) 7.38 (dd, J = 9.35, 2.5 Hz, 1H) |
| | | 7.52 (d, J = 2.5 Hz, 1H). |
| 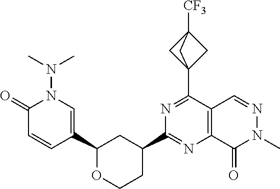 [SMILES: CN(C)n1cc([C@H]2C[C@@H](c3nc(C45CC(C(F)(F)F)(C4)C5)c4cnn(C)c(=O)c4n3)CCO2)ccc1=O] | 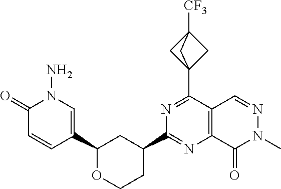 [SMILES: Cn1ncc2c(C34CC(C(F)(F)F)(C3)C4)nc([C@H]3CCO[C@@H](c4ccc(=O)n(N)c4)C3)nc2c1=O] | Modifications: The reaction was run with 2.2 equiv. K₂CO₃ and 4 equiv. MeI in MeCN solvent at 80 °C for 4 days. The crude was then purified by silica gel flash-column chromatography using an elution gradient of 0-10% MeOH in DCM to yield 2-((2R,4S)-2- (1-(dimethylamino)-6-oxo-1,6- dihydropyridin-3-yl)tetrahydro-2H- pyran-4-yl)-7-methyl-4-(3- (trifluoromethyl)bicyclo[1.1.1]pentan- 1-yl)pyrimido[4,5-d]pyridazin-8(7H)- |
| Example 232 | Example 229 | one (Example 232, 7 mg, 0.012 mmol, |
| | | 57% yield) as a solid. ESI-MS: [M + H]⁺ |
| | | 517.3. ¹H NMR (400 MHz, DMSO- |
| | | d₆): δ 8.69 (s, 1H), 7.52 (d, J = 2.3 Hz, |
| | | 1H), 7.40 (dd, J = 9.4, 2.5 Hz, 1H), 6.34 |
| | | (d, J = 9.4 Hz, 1H), 4.33 (d, J = 11.0 Hz, |
| | | 1H), 4.10 (dd, J = 10.9, 3.5 Hz, 1H), |
| | | 3.72 (s, 3H), 3.68-3.63 (m, 1H), 3.40- |
| | | 3.35 (m, 1H), 2.89 (s, 6H), 2.61 (s, 6H), |
| | | 2.11 (d, J = 12.8 Hz, 1H), 1.96-1.86 (m, |
| | | 2H), 1.71 (d, J = 12.3 Hz, 1H). ¹⁹F |
| | | NMR (376 MHz, DMSO-d₆): δ −71.2 |
| | | (s, 3F). |

### Method 35: Synthesis of Example 208

### Example 208:5-((2R,4S)-4-(7-methyl-6-(trifluoromethyl)-4-(3-(trifluoromethyl) bicyclo[1.1.1]pentan-1-yl) pyrido[2,3-d]pyrimidin-2-yl)tetrahydro-2H-pyran-2-yl)-1-(propan-2-ylideneamino)pyridin-2 (1H)-one

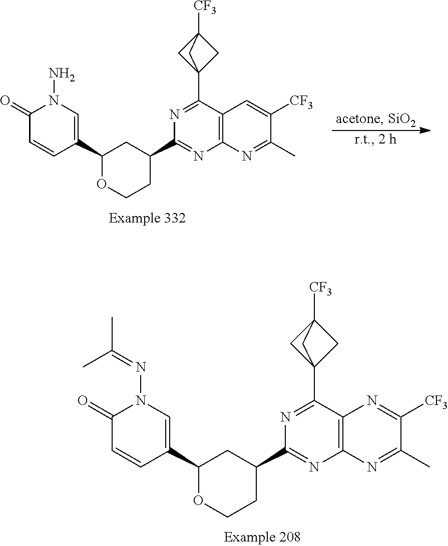 [SMILES: Cc1nc2nc([C@H]3CCO[C@@H](c4ccc(=O)n(N)c4)C3)nc(C34CC(C(F)(F)F)(C3)C4)c2cc1C(F)(F)F ; "acetone, SiO2" ; "r.t., 2 h" ; CC(C)=Nn1cc([C@H]2C[C@@H](c3nc(C45CC(C(F)(F)F)(C4)C5)c4nc(C(F)(F)F)c(C)nc4n3)CCO2)ccc1=O]

To a stirring solution of 1-amino-5-((2R,4S)-4-(7-methyl-6-(trifluoromethyl)-4-(3-(trifluoromethyl) bicyclo[1.1.1]pentan-1-yl) pyrido[2,3-d]pyrimidin-2-yl)tetrahydro-2H-pyran-2-yl)pyridin-2 (1H)-one (Example 332, 1 equiv, 15 mg, 0.026 mmol) in anhydrous acetone (1.0 mL) at r.t., was added silica gel (50 mg). The resulting solution was stirred at r.t. for 2 h, filtered, and concentrated under reduced pressure to yield 5-((2R,4S)-4-(7-methyl-6-(trifluoromethyl)-4-(3-(trifluoromethyl) bicyclo[1.1.1]pentan-1-yl) pyrido[2,3-d]pyrimidin-2-yl)tetrahydro-2H-pyran-2-yl)-1-(propan-2-ylideneamino)pyridin-2 (1H)-one (Example 208, 14 mg, 0.022 mmol, 85% yield) as a solid. ESI-MS: [M+H]⁺=580.3. ¹H NMR (CD₃OD, 400 MHz): δ 8.91 (s, 1H), 7.63 (dd, J=9.4, 2.5 Hz, 1H), 7.50 (d, J=2.5 Hz, 1H), 6.63 (d, J=9.4 Hz, 1H), 4.47 (d, J=9.8 Hz, 1H), 4.26-4.22 (m, 1H), 3.81 (td, J=11.3, 3.7 Hz, 1H), 3.53-3.45 (m, 1H), 2.89 (d, J=1.1 Hz, 3H), 2.68 (s, 6H), 2.31 (d, J=13.3 Hz, 1H), 2.25 (s, 3H), 2.13-1.92 (m, 3H), 1.81 (d, J=4.1 Hz, 3H). ¹⁹F NMR (CD₃OD, 376 MHz): δ −63.7 (s, 3F), -74.6 (s, 3F).

### Method 36: Synthesis of Example 210

### Example 210:1-(2,5-dihydropyrrol-1-yl)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one

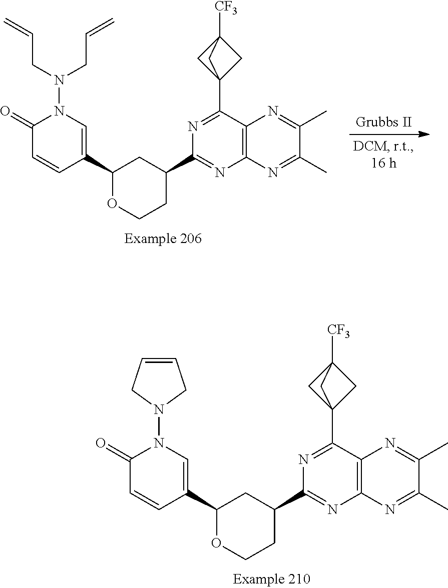 [SMILES: C=CCN(CC=C)n1cc([C@H]2C[C@@H](c3nc(C45CC(C(F)(F)F)(C4)C5)c4nc(C)c(C)nc4n3)CCO2)ccc1=O ; Cc1nc2nc([C@H]3CCO[C@@H](c4ccc(=O)n(N5CC=CC5)c4)C3)nc(C34CC(C(F)(F)F)(C3)C4)c2nc1C]

DCM (1 mL) was added to a round bottomed flask containing 1-(diallylamino)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 206, 1 equiv., 14 mg, 0.02 mmol) and Grubbs Catalyst 2nd generation (0.2 equiv., 4.0 mg, 4.7 μmol) under argon. The reaction mixture was stirred at r.t. for 16 h, concentrated under reduced pressure, and then purified directly by flash chromatography (12 g SiO₂ column) using an elution gradient of 0-100% EtOAc/EtOH (3:1) in hexanes to yield 1-(2,5-dihydropyrrol-1-yl)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 210, 4.0 mg, 7.4 μmol, 32% yield) as a solid. ESI-MS: [M+H]⁺=539.3. 1H NMR (CDCl₃, 400 MHz): δ 7.61 (d, J=2.3 Hz, 1H), 7.41 (dd, J=9.4, 2.7 Hz, 1H), 6.59 (d, J=9.4 Hz, 1H), 5.83 (s, 2H), 4.33-4.26 (m, 2H), 4.20 (s, 4H), 3.79 (td, J=11.5, 3.1 Hz, 1H), 3.45 (t, J=4.0 Hz, 1H), 2.81 (s, 3H), 2.77 (s, 3H), 2.65 (s, 6H), 2.31 (d, J=13.5 Hz, 1H), 2.15-2.02 (m, 3H). ¹⁹F NMR (CDCl₃, 376 MHz): δ −73.0 (s, 3F).

### Method 37: Synthesis of Example 213

### Example 213: N-[5-[(2R,4S)-4-[6,7-dimethyl-4-[3-(trifluoromethyl)-1-bicyclo[1.1.1]pentanyl]pteridin-2-yl]tetrahydropyran-2-yl]-2-oxo-1-pyridyl]prop-2-enamide

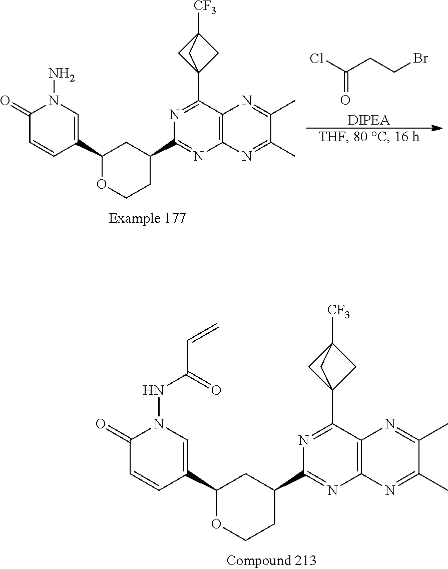

A reaction vial equipped with a Teflon-coated stirring bar was charged with 1-amino-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 177, 1 equiv., 50 mg, 0.1 mmol), DIPEA (2 equiv., 36 μL, 0.21 mmol) and THF (1 mL). The vial was sealed and stirred at 80° C. for 2 h. The reaction mixture was heated to 80° C. for 16 h. The reaction mixture was cooled down to r.t., water was added, and the aqueous phase was extracted three times with EtOAc. Organic phases were combined, dried over anhydrous Na₂SO₄, filtered, and evaporated under reduced pressure. The residue was purified, first, by normal phase flash chromatography (12 g SiO₂), using an elution gradient of 0-100% EtOAc/EtOH (3:1) in hexanes, and second, by reverse phase flash chromatography (6 g C18) using an elution gradient of 20-80% CH₃CN in 0.1% formic acid (aq.) to yield N-[5-[(2R,4S)-4-[6,7-dimethyl-4-[3-(trifluoromethyl)-1-bicyclo[1.1.1]pentanyl]pteridin-2-yl]tetrahydropyran-2-yl]-2-oxo-1-pyridyl]prop-2-enamide (Example 213, 2.5 mg, 4.6 μmol, 4% yield). ESI-MS: [M+H]⁺+=541.3. ¹H NMR (400 MHz, CD₃OD): δ 8.49-8.59 (OH), 7.77 (s, OH), 7.64 (d, J=9.6 Hz, 1H), 7.53 (s, 1H), 6.63 (d, J=9.4 Hz, 1H), 6.57 (d, J=9.4 Hz, OH), 6.39 (d, J=5.7 Hz, 2H), 5.84 (t, J=6.1 Hz, 1H), 4.42 (d, J=11.2 Hz, 1H), 4.23 (d, J=11.2 Hz, 1H), 3.81 (d, J=11.9 Hz, 1H), 3.46 (s, 1H), 2.77 (d, J=3.0 Hz, 7H), 2.64 (s, 6H), 2.28 (d, J=13.0 Hz, 1H), 2.07-1.94 (m, 3H). ¹⁹F NMR (376 MHz, CD₃OD): δ −74.7 (s, 3F).

### Method 38: Synthesis of Example 216

### Example 216:2,3-dimethyl-6-[rac-(2R,4S)-2-(6-oxo-1H-pyridin-3-yl)tetrahydropyran-4-yl]-8-[3-(trifluoromethyl)-1-bicyclo[1.1.1]pentanyl]pyrimido[5,4-d]pyrimidin-4-one

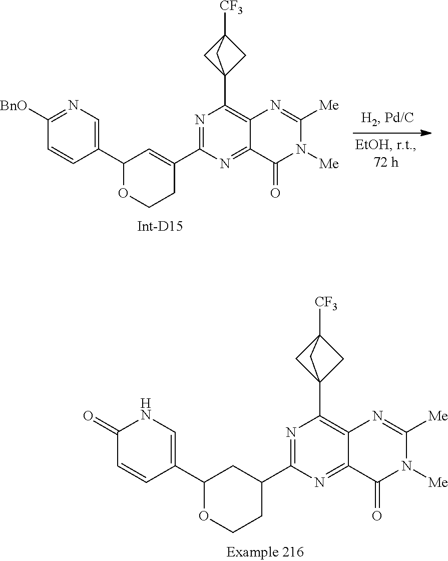 [SMILES: Cc1nc2c(C34CC(C(F)(F)F)(C3)C4)nc(C3=CC(c4ccc(OCc5ccccc5)nc4)OCC3)nc2c(=O)n1C ; Cc1nc2c(C34CC(C(F)(F)F)(C3)C4)nc(C3CCOC(c4ccc(=O)[nH]c4)C3)nc2c(=O)n1C]

A 100 mL round-bottomed flask equipped with a Teflon-coated stirring bar was charged with Pd/C (10 wt %, 0.1 equiv., 13 mg, 0.013 mmol) and purged with argon three times. A solution of 6-[6-(6-benzyloxy-3-pyridyl)-3,6-dihydro-2H-pyran-4-yl]-2,3-dimethyl-8-[3-(trifluoromethyl)-1-bicyclo[1.1.1]pentanyl]pyrimido[5,4-d]pyrimidin-4-one (Int-D15, 1 equiv., 73 mg, 0.13 mmol) in EtOH (1.3 mL) was added. The reaction vial was purged with H₂ (g) and vacuum three times, equipped with a H₂ balloon (1 atm), and stirred at r.t. for 72 h. The reaction mixture was purged with argon, filtered through a Celite pad, rinsed with DCM, and concentrated under reduced pressure. The residue was purified by prep-HPLC (Condition 10, Gradient e) to yield 2,3-dimethyl-6-[rac-(2R,4S)-2-(6-oxo-1H-pyridin-3-yl)tetrahydropyran-4-yl]-8-[3-(trifluoromethyl)-1-bicyclo[1.1.1]pentanyl]pyrimido[5,4-d]pyrimidin-4-one (Example 216, 19 mg, 0.04 mmol, 30% yield) as a solid. ESI-MS: [M+H]⁺=488.4. 1H NMR (CDCl₃, 400 MHz): δ 7.53 (dd, J=9.1, 2.5 Hz, 1H), 7.36 (d, J=1.6 Hz, 1H), 6.58 (d, J=9.4 Hz, 1H), 4.29 (dd, J=11.3, 1.5 Hz, 1H), 4.25 (ddd, J=11.8, 4.5, 1.3 Hz, 1H), 3.74 (td, J=12.0, 2.3 Hz, 1H), 3.66 (s, 3H), 3.47 (tt, J=11.9, 3.8 Hz, 1H), 2.65 (s, 3H), 2.57 (s, 6H), 2.19-1.87 (m, 4H). ¹⁹F NMR (CDCl₃, 376 MHz): δ −73.6 (3F).

### Method 39: Synthesis of Example 227

### Example 227:7-methyl-2-((2R,4S)-2-(6-oxo-1,6-dihydropyridin-3-yl)tetrahydro-2H-pyran-4-yl)-4-(3-(trifluoromethyl) bicyclo[1.1.1]pentan-1-yl) pyrimido[4,5-d]pyridazin-8 (7H)-one

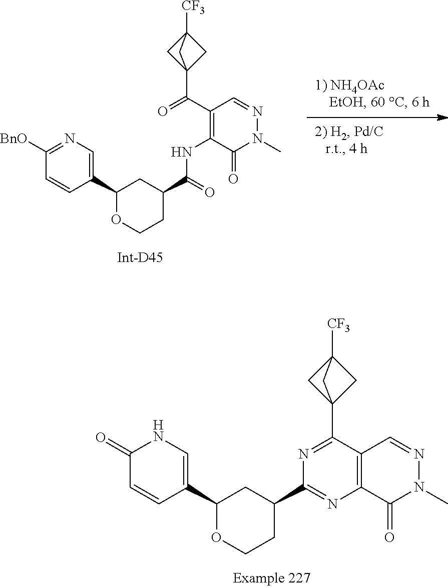

To a stirring solution of methyl(2R,4S)-2-(6-(benzyloxy)pyridin-3-yl)-N-(2-methyl-3-oxo-5-(3-(trifluoromethyl) bicyclo[1.1.1]pentane-1-carbonyl)-2,3-dihydropyridazin-4-yl)tetrahydro-2H-pyran-4-carboxamide (Int-D45, 1 equiv., 93 mg, 0.16 mmol) in EtOH (50 mL), was added ammonium acetate (10 equiv., 126 mg, 1.63 mmol) at r.t . . . . The resulting solution was stirred at 60° C. for 6 h. The reaction mixture was cooled to r.t., degassed with Ar, and charged with 10 wt % Pd/C (0.1 equiv., 17 mg, 0.02 mmol). The reaction mixture was stirred under an atmosphere of H₂, at r.t. for 4 h. The reaction mixture was degassed with Ar and filtered through a short pad of Celite, washing with EtOH. The filtrate was concentrated in vacuo and purified by SiO₂ flash column chromatography using an elution gradient of 1-8% MeOH in DCM to afford 7-methyl-2-((2R,4S)-2-(6-oxo-1,6-dihydropyridin-3-yl)tetrahydro-2H-pyran-4-yl)-4-(3-(trifluoromethyl) bicyclo[1.1.1]pentan-1-yl) pyrimido[4,5-d]pyridazin-8 (7H)-one (Example 227, 57 mg, 0.12 mmol, 57% yield) as a solid. ESI-MS: [M+H]⁺=474.3. ¹H NMR (400 MHz, DMSO-d₆): δ 11.45 (s, 1H), 8.69 (s, 1H), 7.44 (dd, J=9.4, 2.5 Hz, 1H), 7.26 (d, J=2.3 Hz, 1H), 6.27 (d, J=9.4 Hz, 1H), 4.32 (d, J=9.8 Hz, 1H), 4.10 (dd, J=11.2, 3.4 Hz, 1H), 3.71 (s, 3H), 3.65 (td, J=11.7, 2.3 Hz, 1H), 3.37 (qd, J=7.9, 3.9 Hz, 1H), 2.62 (s, 6H), 2.08 (d, J=13.0 Hz, 1H), 1.95-1.83 (m, 2H), 1.75-1.66 (m, J=9.1 Hz, 1H). ¹⁹F NMR (376 MHz, DMSO-d₆): δ −71.2 (s, 3F).

### Method 40: Synthesis of Example 230

### Example 230:2-((2R,4S)-2-(1-cyclopropyl-6-oxo-1,6-dihydropyridin-3-yl)tetrahydro-2H-pyran-4-yl)-7-methyl-4-(3-(trifluoromethyl) bicyclo[1.1.1]pentan-1-yl) pyrimido[4,5-d]pyridazin-8 (7H)-one

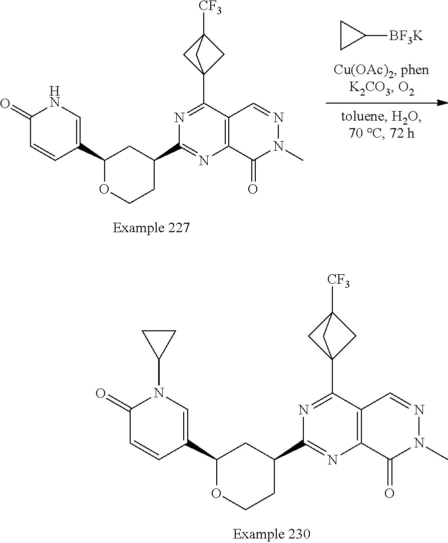

To a stirring solution of 7-methyl-2-((2R,4S)-2-(6-oxo-1,6-dihydropyridin-3-yl)tetrahydro-2H-pyran-4-yl)-4-(3-(trifluoromethyl) bicyclo[1.1.1]pentan-1-yl) pyrimido[4,5-d]pyridazin-8 (7H)-one (Example 227, 1 equiv., 12 mg, 0.025 mmol), potassium cyclopropyl (trifluoro) boranuide (3 equiv., 11 mg, 0.076 mmol), K₂CO₃ (2 equiv., 7 mg, 0.051 mmol), and 1-10-phenanthroline (0.5 equiv., 2.3 mg, 0.013 mmol) in toluene (1.0 mL) and H₂O (0.5 mL) was added Cu(OAc)₂ (1 equiv., 4.6 mg, 0.0.25 mmol). The resulting mixture was purged with oxygen for 5 min, capped, and stirred at 70° C. for 72 h. The reaction mixture was cooled to r.t. and diluted with EtOAc and water. The layers were separated, and the organic layer was washed with brine, dried over anhydrous Na₂SO₄, filtered, and concentrated in vacuo. The crude mixture was purified by silica gel flash column chromatography using an elution gradient of 1-10% MeOH in DCM to yield 2-((2R,4S)-2-(1-cyclopropyl-6-oxo-1,6-dihydropyridin-3-yl)tetrahydro-2H-pyran-4-yl)-7-methyl-4-(3-(trifluoromethyl) bicyclo[1.1.1]pentan-1-yl) pyrimido[4,5-d]pyridazin-8 (7H)-one (Example 230, 5 mg, 9 μmol, 37% yield) as a solid. ESI-MS: [M+H]⁺=514.3. ¹H NMR (400 MHz, CDCl₃): δ 8.43 (s, 1H), 7.35-7.31 (m, 2H), 6.56-6.52 (m, 1H), 4.28-4.25 (m, 2H), 3.89 (s, 3H), 3.73 (td, J=11.9, 2.3 Hz, 1H), 3.55 (tt, J=11.9, 3.7 Hz, 1H), 3.33-3.27 (m, 1H), 2.60 (s, 6H), 2.18 (d, J=12.8 Hz, 1H), 2.14-2.06 (m, 1H), 2.03-2.00 (m, 1H), 1.89 (dd, J=24.7, 11.9 Hz, 1H), 1.14-1.07 (m, 2H), 0.89-0.84 (m, 2H). ¹⁹F NMR (376 MHz, CDCl₃): δ −73.0 (d, J=5.8 Hz, 3F).

An analogous procedure was followed to obtain the following compounds.

| Structure | Intermediate | Characterization |
|---|---|---|
| 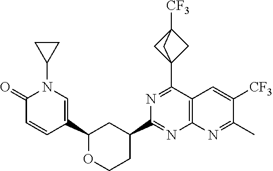 [SMILES: Cc1nc2nc([C@H]3CCO[C@@H](c4ccc(=O)n(C5CC5)c4)C3)nc(C34CC(C(F)(F)F)(C3)C4)c2cc1C(F)(F)F] | 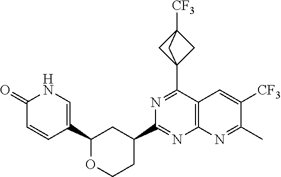 [SMILES: Cc1nc2nc([C@H]3CCO[C@@H](c4ccc(=O)[nH]c4)C3)nc(C34CC(C(F)(F)F)(C3)C4)c2cc1C(F)(F)F] | The crude was purified by flash chromatography (20-50% acetone in hexanes gradient elution) to yield 1-cyclopropyl-5-((2R,4S)-4- (7-methyl-6-(trifluoromethyl)-4- (3- (trifluoromethyl)bicyclo[1.1.1]- pentan-1-yl)pyrido[2,3- d]pyrimidin-2-yl)tetrahydro-2H- pyran-2-yl)pyridin-2(1H)-one (Example 328, 18 mg, 0.032 |
| Example 328 | Example 330 | mmol, 21% yield) as a solid. ESI- |
| | | MS: [M + H]⁺ = 565.3. ¹H NMR |
| | | (400 MHz, CD₃OD): δ 8.91 (s, |
| | | 1H), 7.62-7.58 (m, 2H), 6.52 (d, J = |
| | | 9.1 Hz, 1H), 4.43 (dd, J = 11.3, |
| | | 1.7 Hz, 1H), 4.26-4.22 (m, 1H), |
| | | 3.84-3.77 (m, 1H), 3.52-3.44 (m, |
| | | 1H), 3.35-3.30 (m, 1H), 2.90 (d, J = |
| | | 1.1 Hz, 3H), 2.68 (s, 7H), 2.25 |
| | | (d, J = 13.0 Hz, 1H), 2.10-2.04 (m, |
| | | 2H), 1.96 (dd, J = 24.7, 11.9 Hz, |
| | | 1H), 1.14-1.09 (m, 2H), 0.92-0.88 |
| | | (m, 2H). ¹⁹F NMR (376 MHz, |
| | | CD₃OD): δ −63.7 (s, 3F), −74.6 (d, J = |
| | | 10.0 Hz, 3F). |

### Method 41: Synthesis of Example 336 and 338

### Example 336:1-(dimethylamino)-5-((2R,4S)-4-(7-methyl-6-(trifluoromethyl)-4-(3-(trifluoromethyl) bicyclo-[1.1.1]pentan-1-yl) pyrido[2,3-d]pyrimidin-2-yl)tetrahydro-2H-pyran-2-yl)pyridin-2 (1H)-one; and Example 338:1-(methylamino)-5-((2R,4S)-4-(7-methyl-6-(trifluoromethyl)-4-(3-(trifluoromethyl) bicyclo-[1.1.1]pentan-1-yl) pyrido[2,3-d]pyrimidin-2-yl)tetrahydro-2H-pyran-2-yl)pyridin-2 (1H)-one

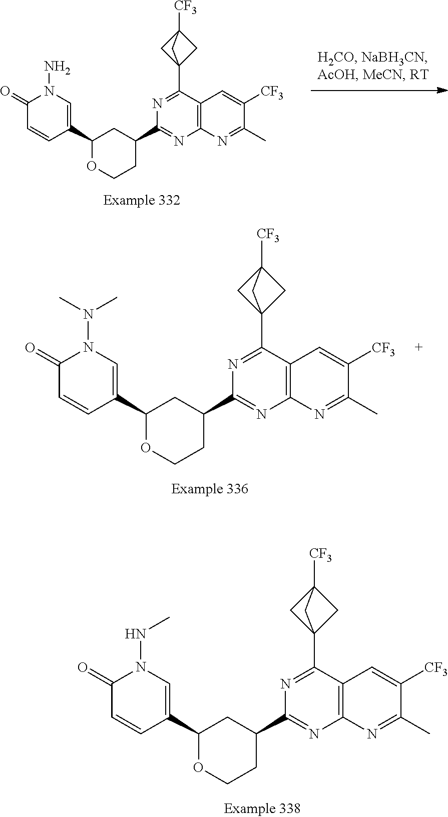

To a stirring solution of 1-amino-5-[(2R,4S)-4-[7-methyl-6-(trifluoromethyl)-4-[3-(trifluoromethyl)-1-bicyclo[1.1.1]pentanyl]pyrido[2,3-d]pyrimidin-2-yl]tetrahydropyran-2-yl]pyridin-2 (1H)-one (Example 332, 1 equiv, 25 mg, 0.046 mmol), paraformaldehyde (5 equiv, 0.017 mL, 0.23 mmol), and acetic acid (1.5 equiv, 40 μL, 0.007 mmol) in MeCN (1.0 mL) at r.t., was added sodium cyanoborohydride (2 equiv, 5.8 mg, 0.09 mmol). The reaction mixture was stirred at this temperature for 2 days. The reaction mixture was diluted with EtOAc, and the resulting solution was washed with sat. NaHCO₃(aq), and brine. The organic layer was dried over anhydrous Na₂SO₄, filtered and concentrated in vacuo. The crude mixture was purified by silica gel flash column chromatography (20-50% acetone in hexanes) to yield 1-(dimethylamino)-5-((2R,4S)-4-(7-methyl-6-(trifluoromethyl)-4-(3-(trifluoromethyl) bicyclo-[1.1.1]pentan-1-yl) pyrido[2,3-d]pyrimidin-2-yl)tetrahydro-2H-pyran-2-yl)pyridin-2 (1H)-one (Example 336) and 1-(methylamino)-5-((2R,4S)-4-(7-methyl-6-(trifluoromethyl)-4-(3-(trifluoromethyl) bicyclo-[1.1.1]pentan-1-yl) pyrido[2,3-d]pyrimidin-2-yl)tetrahydro-2H-pyran-2-yl)pyridin-2 (1H)-one (Example 338) as solids.

Example 336: ESI-MS: [M+H]⁺568.3. ¹H NMR (400 MHz, CD₃OD): δ 8.91 (s, 1H), 8.50 (s, 1H), 7.73 (d, J=2.5 Hz, 1H), 7.57 (dd, J=9.4, 2.5 Hz, 1H), 6.52 (d, J=9.1 Hz, 1H), 4.42 (dd, J=11.3, 1.7 Hz, 1H), 4.26-4.22 (m, 1H), 3.84-3.77 (m, 1H), 3.49-3.46 (m, 1H), 2.95 (s, 6H), 2.90 (d, J=1.1 Hz, 3H), 2.68 (d, J=5.9 Hz, 6H), 2.27 (d, J=13.3 Hz, 1H), 2.10-2.04 (m, 2H), 1.96 (dd, J=24.8, 11.8 Hz, 1H). ¹⁹F NMR (376 MHz, CD₃OD): δ −63.7 (s, 3F), -74.6 (s, 3F).

Example 338: ESI-MS: 554.3. ¹H NMR (400 MHz, CD₃OD): δ 8.91 (s, 1H), 8.50 (s, 1H), 7.80 (d, J=2.5 Hz, 1H), 7.61-7.58 (m, 1H), 6.60 (d, J=9.4 Hz, 1H), 4.45 (dd, J=11.4, 1.8 Hz, 1H), 4.27-4.23 (m, 1H), 3.85-3.78 (m, 1H), 3.52-3.45 (m, 1H), 2.90 (d, J=1.1 Hz, 3H), 2.77 (s, 3H), 2.68 (d, J=6.6 Hz, 6H), 2.29 (d, J=13.3 Hz, 1H), 2.10-2.05 (m, 2H), 1.96 (dd, J=24.8, 11.8 Hz, 1H). ¹⁹F NMR (376 MHz, CD₃OD): δ −63.7 (s, 3F), -74.6 (s, 3F).

### Method 42: Synthesis of Example 311 and 322

### Example 311:5-((2R,4S)-4-(6-chloro-7-methyl-4-(3-(trifluoromethyl) bicyclo[1.1.1]pentan-1-yl) pyrido[2,3-d]pyrimidin-2-yl)tetrahydro-2H-pyran-2-yl)-1-(methyl-d₃)pyridin-2 (1H)-one; and

### Example 322:1-(methyl-d₃)-5-((2R,4S)-4-(7-methyl-4-(3-(trifluoromethyl) bicyclo[1.1.1]pentan-1-yl) pyrido[2,3-d]pyrimidin-2-yl)tetrahydro-2H-pyran-2-yl)pyridin-2 (1H)-one

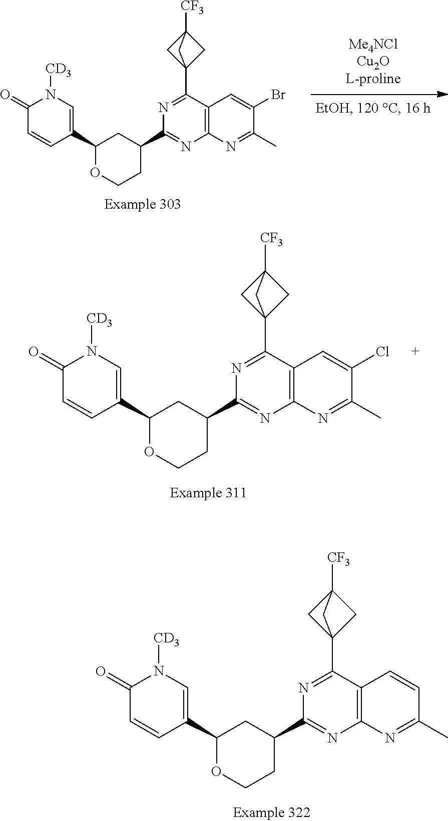 [SMILES: [2H]C([2H])([2H])n1cc([C@H]2C[C@@H](c3nc(C45CC(C(F)(F)F)(C4)C5)c4cc(Br)c(C)nc4n3)CCO2)ccc1=O ; "EtOH, 120 °C, 16 h" ; "Me4NClCu2OL-proline" ; [2H]C([2H])([2H])n1cc([C@H]2C[C@@H](c3nc(C45CC(C(F)(F)F)(C4)C5)c4cc(Cl)c(C)nc4n3)CCO2)ccc1=O ; [2H]C([2H])([2H])n1cc([C@H]2C[C@@H](c3nc(C45CC(C(F)(F)F)(C4)C5)c4ccc(C)nc4n3)CCO2)ccc1=O]

A flame-dried microwave vial was charged with 5-[(2R,4S)-4-[6-bromo-7-methyl-4-[3-(trifluoromethyl)-1-bicyclo[1.1.1]pentanyl]pyrido[2,3-d]pyrimidin-2-yl]tetrahydropyran-2-yl]-1-(trideuteriomethyl)pyridin-2-one (Example 303, 1 equiv., 60 mg, 0.11 mmol), Cu₂O (0.2 equiv., 3.1 mg, 0.022 mmol), Me₄NCl (4 equiv., 48 mg, 0.43 mmol) and ethanol (2.2 mL). The mixture was stirred at 120° C. for 16 h. Sat. NaHCO₃ (20 ml) was added, and the reaction was extracted with EtOAc (2×20 ml). The combined organic layers were washed with brine (10 mL), dried over Na₂SO₄, filtered, and concentrated under reduced pressure. The residue was purified by normal phase chromatography, 30-100% EtOAc in hexanes, then 0-20% MeOH in DCM. The material was further purified by preparative HPLC (Condition 14, Gradient b) to afford the desired product 5-((2R,4S)-4-(6-chloro-7-methyl-4-(3-(trifluoromethyl) bicyclo[1.1.1]pentan-1-yl) pyrido[2,3-d]pyrimidin-2-yl)tetrahydro-2H-pyran-2-yl)-1-(methyl-d₃)pyridin-2 (1H)-one (Example 311, 9.5 mg, 17% yield) and 1-(methyl-d₃)-5-((2R,4S)-4-(7-methyl-4-(3-(trifluoromethyl) bicyclo[1.1.1]-pentan-1-yl) pyrido[2,3-d]pyrimidin-2-yl)tetrahydro-2H-pyran-2-yl)pyridin-2 (1H)-one (Example 322, 5.5 mg, 11% yield) as solids.

Example 311: ESI-MS: [M+H]⁺=508.3. ¹H NMR (CDCl₃, 400 MHz): δ 8.42 (1H, s), 7.36-7.39 (2H, m), 6.53-6.56 (1H, m), 4.24-4.31 (2H, m), 3.73-3.80 (1H, m), 3.38-3.46 (1H, m), 2.88 (3H, s), 2.62 (6H, s), 2.26-2.31 (1H, m), 2.06-2.14 (2H, m), 1.95-2.04 (1H, m). ¹⁹F NMR (CDCl₃, 376 MHz): δ −73.1 (3F, s).

Example 322: ESI-MS: [M+H]⁺=473.51. ¹H NMR (CDCl₃, 400 MHz): δ 8.43 (1H, d, J=8.5 Hz), 7.37-7.41 (3H, m), 6.55 (1H, d, J=9.4 Hz), 4.25-4.32 (2H, m), 3.74-3.81 (1H, m), 3.38-3.46 (1H, m), 2.82 (3H, s), 2.61 (6H, s), 2.29-2.33 (1H, m), 2.06-2.13 (2H, m), 1.98-2.04 (1H, m). ¹⁹F NMR (CDCl₃, 376 MHz): δ −73.2 (3F, s).

### Method 43: Synthesis of Example 295

### Example 295:5-((2R,4S)-4-(6-chloro-7-methyl-4-(3-(trifluoromethyl) bicyclo[1.1.1]pentan-1-yl) pyrido[2,3-d]pyrimidin-2-

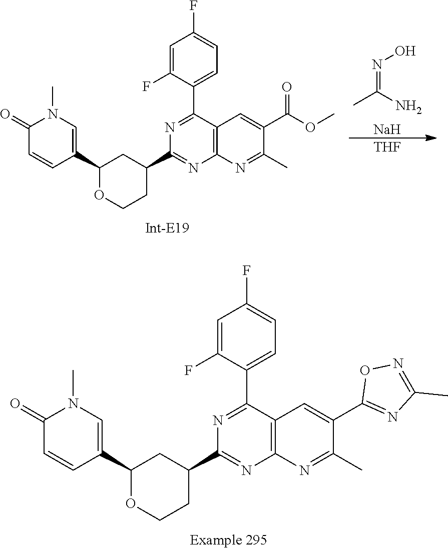

To a solution of methyl 4-(2,4-difluorophenyl)-7-methyl-2-[(2R,4S)-2-(1-methyl-6-oxo-3-pyridyl)tetrahydropyran-4-yl]pyrido[2,3-d]pyrimidine-6-carboxylate (Int-E19, 1 equiv., 35 mg, 0.07 mmol) and N′-hydroxyacetamidine (1.2 equiv., 6.1 mg, 0.083 mmol) in THF (1 mL) was added sodium hydride (1.2 eq, 3.3 mg, 0.083 mmol). The resulting mixture was stirred at 50° C. for 1 h. The reaction was quenched by the addition of a solution of NH₄Cl (10 mL) and EtOAc (10 mL). The organic phase was washed with brine, filtered, and concentrated under reduced pressure. The crude material was purified by flash chromatography using a 0-20% MeOH in DCM elution gradient followed by reverse phase chromatography using a 25-85% MeCN in water (containing 0.1% formic acid) gradient to afford 5-((2R,4S)-4-(4-(2,4-difluorophenyl)-7-methyl-6-(3-methyl-1,2,4-oxadiazol-5-yl) pyrido[2,3-d]pyrimidin-2-yl)tetrahydro-2H-pyran-2-yl)-1-methylpyridin-2 (1H)-one (Example 295, 9 mg, 0.017 mmol, 25%) ¹H NMR (CDCl₃, 400 MHz): δ 8.79 (1H, d, J=4.0 Hz), 7.69 (1H, td, J=8.3, 6.2 Hz), 7.36-7.39 (2H, m), 7.17 (1H, td, J=8.2, 2.4 Hz), 7.06-7.11 (1H, m), 6.54-6.57 (1H, m), 4.27-4.34 (2H, m), 3.75-3.82 (1H, m), 3.54 (3H, s), 3.20 (3H, s), 2.51 (3H, s), 2.34-2.39 (1H, m), 2.08-2.24 (3H, m). 19F NMR (CDCl₃, 376 MHz): δ −104.2 (1F, s), -108.8 (1F, s). ESI-MS: [M+H]⁺=531.3.

### Method 44: Synthesis of Example 309

### Example 309:1-(difluoromethyl)-5-((2R,4S)-4-(6-fluoro-7-methyl-4-(3-(trifluoromethyl) bicyclo[1.1.1]pentan-1-yl) pyrido[2,3-d]pyrimidin-2-yl)tetrahydro-2H-pyran-2-yl)pyridin-2 (1H)-one

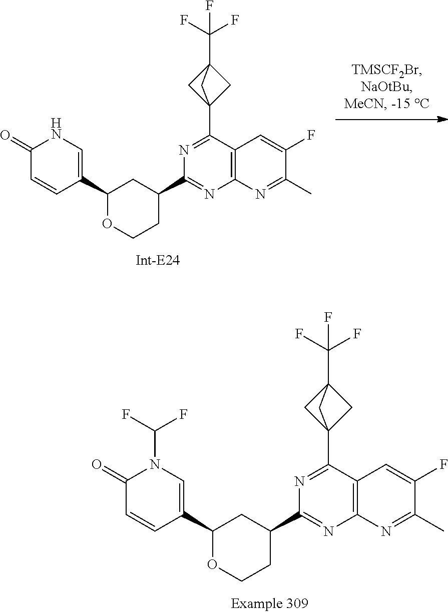 [SMILES: Cc1nc2nc([C@H]3CCO[C@@H](c4ccc(=O)[nH]c4)C3)nc(C34CC(C(F)(F)F)(C3)C4)c2cc1F ; "TMSCF2Br,NaOtBu,MeCN, -15 °C" ; Cc1nc2nc([C@H]3CCO[C@@H](c4ccc(=O)n(C(F)F)c4)C3)nc(C34CC(C(F)(F)F)(C3)C4)c2cc1F]

5-[(2R,4S)-4-[6-fluoro-7-methyl-4-[3-(trifluoromethyl)-1-bicyclo[1.1.1]pentanyl]pyrido[2,3-d]pyrimidin-2-yl]tetrahydropyran-2-yl]-1H-pyridin-2-one (Int-E24, 1 equiv., 45 mg, 0.1 mmol) and sodium tert-butoxide (2.2 equiv., 1.1 mmol, 106 mg) were added into an oven-dried vial under argon. MeCN (0.4 mL) was added under argon at room temperature, and the solution was stirred for 10 min at-15° C. (NH₄Cl/ice bath). TMSCF₂Br (1.2 eq, 18 μL, 0.1 mmol) in 50 μL of MeCN was added dropwise, and the solution was stirred for 1 h at −15° C. Subsequently, the reaction mixture was warmed to room temperature, diluted with brine (15 mL), and extract with EtOAc (3×15 mL). The combined organic layer was washed with brine, dried over Na₂SO₄, filtered, and concentrated under reduced pressure. The crude material was purified by reverse phase flash chromatography (Condition 9, Gradient b) to afford 1-(difluoromethyl)-5-((2R,4S)-4-(6-fluoro-7-methyl-4-(3-(trifluoromethyl) bicyclo[1.1.1]pentan-1-yl) pyrido[2,3-d]pyrimidin-2-yl)tetrahydro-2H-pyran-2-yl)pyridin-2 (1H)-one (Example 309, 7.0 mg, 0.01 mmol, 14% yield). ¹H NMR (CDCl₃, 400 MHz): δ 8.04 (1H, d, J=8.5 Hz), 7.69 (1H, t, J=60.3 Hz), 7.44-7.49 (2H, m), 6.54-6.56 (1H, m), 4.28-4.35 (2H, m), 3.75-3.82 (1H, m), 3.40-3.48 (1H, m), 2.81 (4H, d, J=2.9 Hz), 2.63 (7H, s), 2.33 (1H, d, J=13.3 Hz), 2.10-2.18 (2H, m), 1.94-2.03 (1H, m). 19F NMR (CDCl₃, 376 MHz): δ −73.2 (3F, s), -103.5 (2F, d, J=60.3 Hz), -120.16-120.23 (1F, m). ESI-MS: [M+H]⁺=525.3.

An analogous procedure was followed to obtain the following compounds.

| Structure | Intermediate | Characterization |
|---|---|---|
| 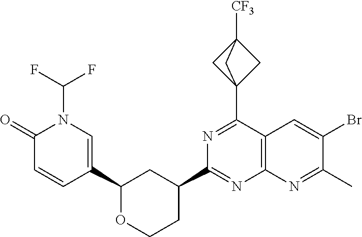 [SMILES: Cc1nc2nc([C@H]3CCO[C@@H](c4ccc(=O)n(C(F)F)c4)C3)nc(C34CC(C(F)(F)F)(C3)C4)c2cc1Br] | 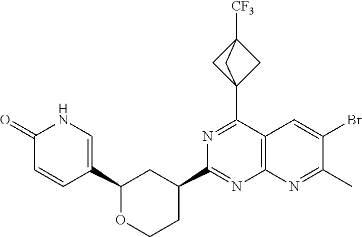 [SMILES: Cc1nc2nc([C@H]3CCO[C@@H](c4ccc(=O)[nH]c4)C3)nc(C34CC(C(F)(F)F)(C3)C4)c2cc1Br] | The crude was purified by normal phase chromatography (50-100% EtOAc in hexanes elution gradient) to provide 5-[(2R,4S)-4-[6-bromo- 7-methyl-4-[3-(trifluoromethyl)-1- bicyclo[1.1.1]pentanyl]pyrido[2,3- d]pyrimidin-2-yl]tetrahydropyran- 2-yl]-1-(difluoromethyl)pyridin-2- one as a solid (Example 313, 41 mg, 19% yield). ¹H NMR (CDCl₃, 400 MHz): δ 8.62 (1H, s), 7.75 (1H, d, J = 60.3 Hz), 7.42-7.51 (3H, m), 6.54 (1H, d, J = 9.6 Hz), 4.26-4.34 (2H, m), 3.73-3.80 (1H, m),3.38-3.46 (1H, m), 2.92 (3H, s), |
| Example 313 | Int-E31 | 2.62 (6H, s), 2.28-2.33 (1H, m), |
| | | 2.08-2.16 (2H, m), 1.99 (1H, q, J = |
| | | 12.3 Hz). ¹⁹F NMR (CDCl₃, 376 |
| | | MHz): δ −73.1, −103.5, −103.6. |
| | | ESI-MS: [M + H]⁺ = 587.2. |
| 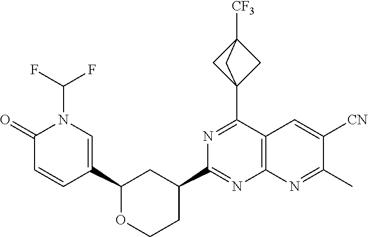 [SMILES: Cc1nc2nc([C@H]3CCO[C@@H](c4ccc(=O)n(C(F)F)c4)C3)nc(C34CC(C(F)(F)F)(C3)C4)c2cc1C#N] | 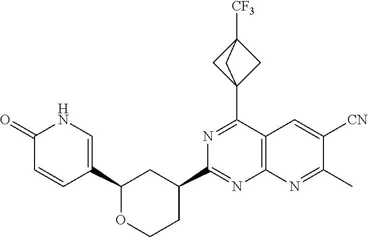 | The crude was purified by normal phase chromatography (50-100% EtOAc in hexanes elution gradient) followed by preparative HPLC (Condition 16, Gradient c) to afford 2-[(2R,4S)-2-[1- (difluoromethyl)-6-oxo-3- pyridyl]tetrahydropyran-4-yl]-7- methyl-4-[3-(trifluoromethyl)-1- bicyclo[1.1.1]pentanyl]pyrido[2,3- d]pyrimidine-6-carbonitrile (Example 315, 3.5 mg, 13% yield) as a solid. ESI-MS: [M + H]⁺ = 532.2. ¹H NMR (CDCl₃, 400 MHz): 8 8.82 (1H, s), 7.69 (1H, t, |
| Example 315 | Int-E34 | J = 60.3 Hz), 7.51 (1H, d, J = 2.3 |
| | | Hz), 7.46 (1H, dd, J = 9.6, 2.5 Hz), |
| | | 6.56 (1H, d, J = 9.6 Hz), 4.28-4.35 |
| | | (2H, m), 3.79 (1H, td, J = 11.6, 3.2 |
| | | Hz), 3.44-3.51 (1H, m), 3.02-3.02 |
| | | (3H, m), 2.67 (6H, s), 2.33 (1H, d, J = |
| | | 13.6 Hz), 2.08-2.15 (2H, m), |
| | | 1.94-2.03 (1H, m). ¹⁹F NMR |
| | | (CDCl₃, 376 MHz): δ −73.1 (3F, s), |
| | | −103.4 (2F, q, J = 60.5 Hz). |
| 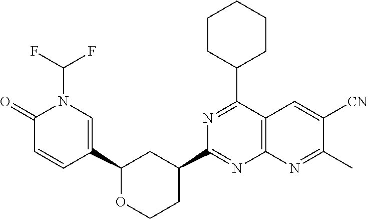 [SMILES: Cc1nc2nc([C@H]3CCO[C@@H](c4ccc(=O)n(C(F)F)c4)C3)nc(C3CCCCC3)c2cc1C#N] | 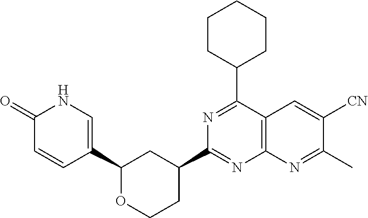 | The residue was purified by flash chromatography using 50-100% EtOAc in DCM gradient to yield 4- cyclohexyl-2-[(2R,4S)-2-[1- (difluoromethyl)-6-oxo-3- pyridyl]tetrahydropyran-4-yl]-7- methyl-pyrido[2,3-d]pyrimidine- 6-carbonitrile (Example 317, 25 mg, 0.052 mmol, 34% yield) as a solid. ESI-MS: [M + H]⁺ = 480.4. ¹H NMR (CDCl₃, 400 MHz): δ 8.76 (1H, s), 7.67 (1H, t, J = 60.3 Hz), 7.50 (1H, s), 7.45 (1H, dd, J = 9.6, 2.4 Hz), 6.54 (1H, d, J = 9.6 |
| Example 317 | Int-E41 | Hz), 4.27-4.34 (2H, m), 3.78 (1H, |
| | | td, J = 11.6, 3.0 Hz), 3.38-3.48 |
| | | (2H, m), 2.99 (3H, s), 2.60 (1H, s), |
| | | 2.33 (1H, d, J = 13.4 Hz), 2.05-2.18 |
| | | (2H, m), 1.76-2.00 (8H, m), 1.35- |
| | | 1.53 (4H, m). ¹⁹F NMR (CDCl₃, |
| | | 376 MHz): δ −103.5 (1F, s), −103.6 |
| | | (1F, s). |
| 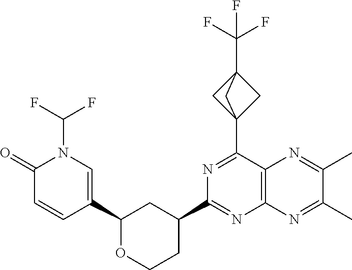 [SMILES: Cc1nc2nc([C@H]3CCO[C@@H](c4ccc(=O)n(C(F)F)c4)C3)nc(C34CC(C(F)(F)F)(C3)C4)c2nc1C] | 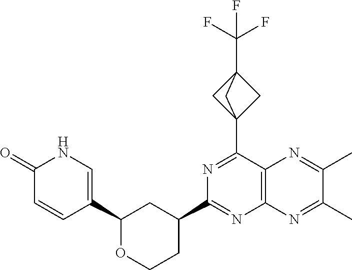 [SMILES: Cc1nc2nc([C@H]3CCO[C@@H](c4ccc(=O)[nH]c4)C3)nc(C34CC(C(F)(F)F)(C3)C4)c2nc1C] | Modification: The reaction was run in diglyme solvent and purified by flash chromatography (24 g SiO₂ column, 10-50% acetone in hexanes elution gradient). Further purification by reverse phase flash chromatography (Condition 9, Gradient b) afforded (1- (difluoromethyl)-5-[(2R,4S)-4- [6,7-dimethyl-4-[3- (trifluoromethyl)-1- bicyclo[1.1.1]pentanyl]pteridin-2- yl]tetrahydropyran-2-yl]pyridin-2- one (Example 90, 7.5 mg, 0.01 mmol, 7% yield) as a solid. ¹H NMR (CDCl₃, 400 MHz): δ 7.38- 7.75 (3H, m), 6.54 (1H, d, J = 9.6 |
| Example 90 | Example 34 | Hz), 4.26-4.34 (2H, m), 3.74-3.81 |
| | | (1H, m), 3.40-3.47 (1H, m), 2.80 |
| | | (3H, s), 2.76 (3H, s), 2.63 (6H, s), |
| | | 2.30-2.34 (1H, m), 2.07-2.14 (2H, |
| | | m), 2.01 (1H, t, J = 12.4 Hz). ¹⁹F |
| | | NMR (CDCl₃, 376 MHz): δ −73.2 |
| | | (3F, s), −103.5 (2F, d, J = 60.3 Hz). |
| | | ESI-MS: [M + H]⁺ = 522.3. |

### Method 45: Synthesis of Example 305

### Example 305:7-methyl-2-((2R,4S)-2-(1-(methyl-d₃)-6-oxo-1,6-dihydropyridin-3-yl)tetrahydro-2H-pyran-4-yl)-4-(3-(trifluoromethyl) bicyclo[1.1.1]pentan-1-yl) pyrido[2,3-d]pyrimidine-6-carbonitrile

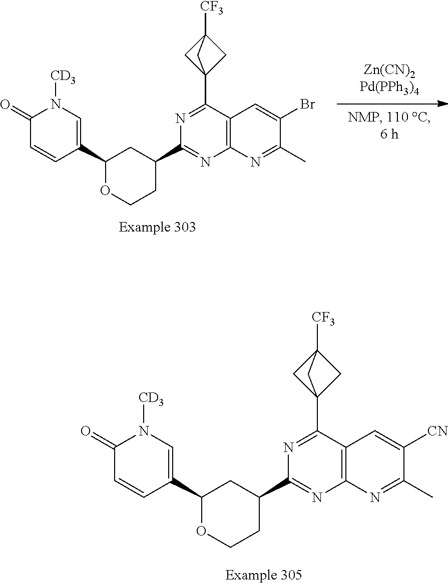 [SMILES: [2H]C([2H])([2H])n1cc([C@H]2C[C@@H](c3nc(C45CC(C(F)(F)F)(C4)C5)c4cc(Br)c(C)nc4n3)CCO2)ccc1=O ; "NMP, 110 °C,6 h" ; [2H]C([2H])([2H])n1cc([C@H]2C[C@@H](c3nc(C45CC(C(F)(F)F)(C4)C5)c4cc(C#N)c(C)nc4n3)CCO2)ccc1=O ; "Zn(CN)2Pd(PPh3)4"]

5-((2R,4S)-4-(6-bromo-7-methyl-4-(3-(trifluoromethyl) bicyclo[1.1.1]pentan-1-yl) pyrido[2,3-d]pyrimidin-2-yl)tetrahydro-2H-pyran-2-yl)-1-(methyl-d₃)pyridin-2 (1H)-one (Example 303, 1 equiv., 60 mg, 0.11 mmol), Zn(CN)₂ (2 equiv., 26 mg, 0.22 mmol), Pd(PPh₃)₄ (0.1 equiv., 13 mg) was stirred at 110° C. in NMP (1.5 mL) for 6 h. The reaction washed with water and extracted with EtOAc (2× 20 mL). The combined organic layers were washed with brine, dried over Na₂SO₄, filtered, and concentrated under reduced pressure. The crude was purified by normal phase chromatography (0-100% EtOAc in hexanes elution gradient, then 0-20% MeOH in DCM elution gradient). The material was further purified by preparative HPLC (Condition 14, Gradient c), to afford 7-methyl-2-((2R,4S)-2-(1-(methyl-d₃)-6-oxo-1,6-dihydropyridin-3-yl)tetrahydro-2H-pyran-4-yl)-4-(3-(trifluoromethyl) bicyclo[1.1.1]pentan-1-yl) pyrido[2,3-d]pyrimidine-6-carbonitrile (Example 305, 22 mg, 41% yield) as a solid. ESI-MS: [M+H]⁺=482.3. ¹H NMR (CDCl₃, 400 MHz): δ 8.81 (1H, s), 7.37 (2H, dd, J=7.1, 2.8 Hz), 6.54-6.57 (1H, m), 4.26-4.32 (2H, m), 3.73-3.80 (1H, m), 3.46 (1H, dd, J=12.1, 10.1 Hz), 3.01 (3H, s), 2.65 (6H, s), 2.29 (1H, d, J=13.3 Hz), 2.07-2.13 (2H, m), 1.93-2.02 (1H, m). ¹⁹F NMR (CDCl₃, 376 MHz): δ −73.2 (3F, s).

### Method 46: Synthesis of Example 297

### Example 297:1-methyl-5-[(2R,4S)-4-[7-methyl-6-(4-methyloxazol-2-yl)-4-[3-(trifluoromethyl)-1-bicyclo[1.1.1]pentanyl]pyrido[2,3-d]pyrimidin-2-yl]tetrahydropyran-2-yl]pyridin-2-one

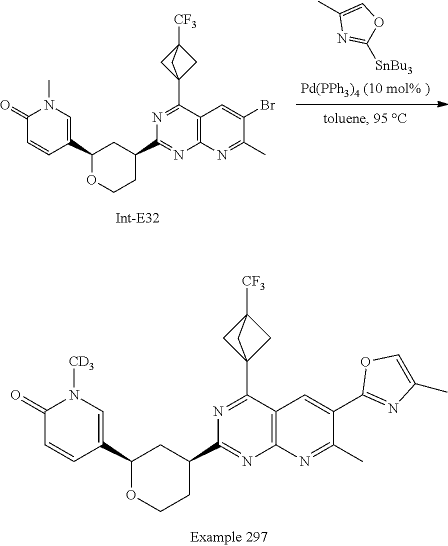

To a flame-dried round-bottomed flask equipped with a Teflon-coated magnetic stirring bar was added 5-[(2R,4S)-4-[6-bromo-7-methyl-4-[3-(trifluoromethyl)-1-bicyclo[1.1.1]pentanyl]pyrido[2,3-d]pyrimidin-2-yl]tetrahydropyran-2-yl]-1-methyl-pyridin-2-one (1 equiv., 45 mg, 0.08 mmol), tributyl-(4-methyloxazol-2-yl) stannane (4 equiv., 122 mg, 0.33 mmol) and Pd(PPh₃)₄ (0.1 equiv., 9.5 mg, 10 mol %). The vial was sealed and purged under argon. Anhydrous toluene (1 mL) was added. The resulting solution was degassed for 2 min with argon and then heated to 95° C. with stirring. After 18 h at 95° C., the reaction mixture was cooled to room temperature and diluted with EtOAc (50 mL). The resulting solution was washed with sat. NH₄Cl (aq) and brine. The organic layer was collected, dried over Na₂SO₄, filtered, and concentrated in vacuo. The crude mixture was purified by reverse phase flash column chromatography (Condition 9, Gradient c) to afford 1-methyl-5-[(2R,4S)-4-[7-methyl-6-(4-methyloxazol-2-yl)-4-[3-(trifluoromethyl)-1-bicyclo[1.1.1]pentanyl]pyrido[2,3-d]pyrimidin-2-yl]tetrahydropyran-2-yl]pyridin-2-one (Example 297, 32 mg, 0.06 mmol, 69% yield) as a powder. 1H NMR (CDCl₃, 400 MHz): δ 9.06 (1H, s), 7.59 (1H, d, J=1.5 Hz), 7.37-7.40 (2H, m), 6.57 (1H, d, J=10.1 Hz), 4.27-4.34 (2H, m), 3.75-3.82 (1H, m), 3.55 (3H, s), 3.42-3.49 (1H, m), 3.17 (3H, s), 2.68 (6H, s), 2.30-2.33 (4H, m), 2.11-2.16 (2H, m), 1.99-2.08 (1H, m). ¹⁹F NMR (CDCl₃, 376 MHz): δ −73.0 (3F, s). ESI-MS: [M+H]⁺=552.3.

### Method 47: Synthesis of Example 299

### Example 299:1-methyl-5-[(2R,4S)-4-[7-methyl-6-(3-methyl-1,2,4-oxadiazol-5-yl)-4-[3-(trifluoromethyl)-1-bicyclo[1.1.1]pentanyl]pyrido[2,3-d]pyrimidin-2-yl]tetrahydropyran-2-yl]pyridin-2-one

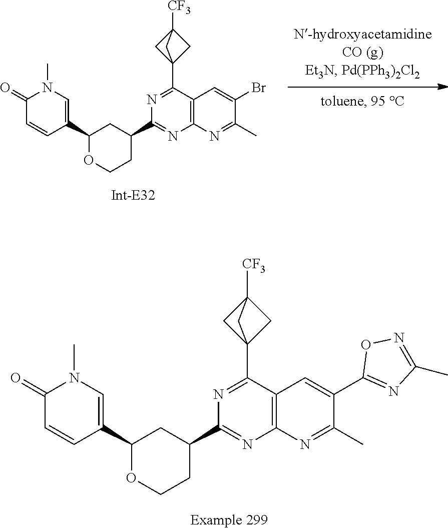

To a flame-dried microwave vial equipped with a Teflon-coated magnetic stirring bar was added N′-hydroxyacetamidine (3 equiv., 18 mg, 0.25 mmol), 5-[(2R,4S)-4-[6-bromo-7-methyl-4-[3-(trifluoromethyl)-1-bicyclo[1.1.1]pentanyl]pyrido[2,3-d]pyrimidin-2-yl]tetrahydropyran-2-yl]-1-methyl-pyridin-2-one (1 equiv., 45 mg, 0.0819 mmol), Pd(PPh₃)₂Cl₂ (0.05 equiv., 2.9 mg) and anh. toluene (0.8 mL). The vial was sealed and purged under argon. Triethylamine (2 equiv., 23 μL, 0.16 mmol) was added and the reaction flask was purged with CO (balloon). The flask was heated to 95° C. with stirring under 1 atm of CO overnight. After 16 h, additional N′-hydroxyacetamidine (3 equiv., 18 mg, 0.25 mmol), Pd(PPh₃)₂Cl₂ (0.05 equiv., 2.9 mg) and triethylamine (2 equiv, 23 μL, 0.16 mmol) were added. The reaction mixture was stirred for 95° C. under CO (balloon) for 2 days. The reaction mixture was cooled to room temperature, diluted with EtOAc, washed with sat. NH₄Cl (aq) and brine, dried over Na₂SO₄, filtered, and concentrated in vacuo. The product was purified by reverse phase chromatography (Condition 9, Gradient d) to give 1-methyl-5-[(2R,4S)-4-[7-methyl-6-(3-methyl-1,2,4-oxadiazol-5-yl)-4-[3-(trifluoromethyl)-1-bicyclo[1.1.1]pentanyl]pyrido[2,3-d]pyrimidin-2-yl]tetrahydropyran-2-yl]pyridin-2-one (Example 299, 14 mg, 0.02 mmol, 29% yield) as a solid. ¹H NMR (CDCl₃, 400 MHz): δ 9.24 (1H, d, J=2.6 Hz), 7.38-7.40 (2H, m), 6.57 (1H, dd, J=9.4, 3.0 Hz), 4.28-4.34 (2H, m), 3.80 (1H, t, J=11.0 Hz), 3.55 (3H, d, J=2.6 Hz), 3.43-3.48 (1H, m), 3.19 (3H, d, J=2.6 Hz), 2.69 (6H, d, J=2.7 Hz), 2.56 (3H, d, J=2.7 Hz), 2.32 (1H, d, J=13.3 Hz), 2.13-2.16 (2H, m), 1.98-2.07 (1H, m). ¹⁹F NMR (CDCl₃, 376 MHz): δ −73.1 (3F, s). ESI-MS: [M+H]⁺=553.3.

### Method 48: Synthesis of Example 118

### Example 118:1-(1-deuteriocyclopropyl)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one

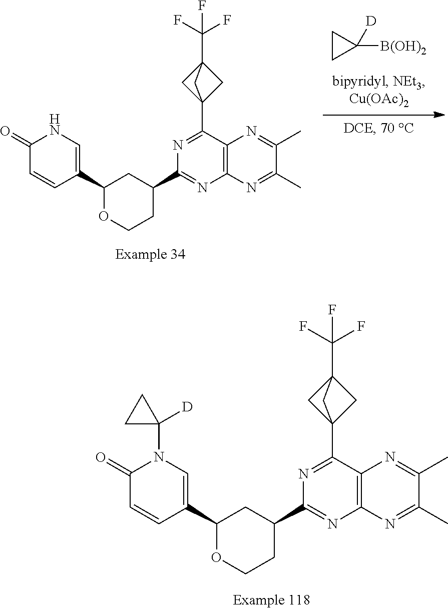 [SMILES: Cc1nc2nc([C@H]3CCO[C@@H](c4ccc(=O)[nH]c4)C3)nc(C34CC(C(F)(F)F)(C3)C4)c2nc1C ; [2H]C1(B(O)O)CC1 ; "DCE, 70 °C" ; "bipyridyl, NEt3,Cu(OAc)2" ; [2H]C1(n2cc([C@H]3C[C@@H](c4nc(C56CC(C(F)(F)F)(C5)C6)c5nc(C)c(C)nc5n4)CCO3)ccc2=O)CC1]

To a microwave vial equipped with a Teflon-coated magnetic stirring bar was added bipyridyl (2.0 equiv., 46 mg, 0.30 mmol) and the vial was flame-dried under vacuum. 5-[(2R,4S)-4-[6,7-dimethyl-4-[3-(trifluoromethyl)-1-bicyclo[1.1.1]pentanyl]pteridin-2-yl]tetrahydropyran-2-yl]-1H-pyridin-2-one (Example 34, 1 equiv., 70 mg, 0.15 mmol), (1-deuteriocyclopropyl) boronic acid (2 equiv., 26 mg, 0.30 mmol), triethylamine (2.0 equiv., 41 μL, 0.30 mmol) and Cu(OAc)₂ (2.0 equiv., 54 mg, 0.30 mmol) were added to the reaction vial, which was then purged under argon. DCE (1.5 mL) was added, and then the reaction mixture was degassed and then heated to 70° C. under stirring for 18 h under argon. The reaction was cooled to room temperature and diluted with EtOAc. The resulting suspension was washed with sat. NH₄Cl (aq.) and brine. The organic layer was collected, dried over Na₂SO₄, filtered, and concentrated in vacuo. The crude reaction mixture was purified by flash chromatography (12 g SiO₂ column, 10-50% acetone in hexanes) to yield 11.5 mg of 1-(1-deuteriocyclopropyl)-5-[(2R,4S)-4-[6,7-dimethyl-4-[3-(trifluoromethyl)-1-bicyclo[1.1.1]pentanyl]pteridin-2-yl]tetrahydropyran-2-yl]pyridin-2-one (Example 118). ¹H NMR (CDCl₃, 400 MHz): δ 7.35-7.37 (2H, m), 6.53 (1H, d, J=9.7 Hz), 4.28 (2H, t, J=9.8 Hz), 3.74-3.80 (1H, m), 3.39-3.47 (1H, m), 2.79 (3H, s), 2.76 (3H, s), 2.63 (7H, s), 2.25-2.29 (1H, m), 2.09-2.14 (2H, m), 1.95-2.04 (1H, m), 1.11 (3H, s), 0.86-0.87 (3H, m). ¹⁹F NMR (CDCl₃, 376 MHz): δ −73.1 (3F, s). ESI-MS: [M+H]⁺=513.3.

### Example A3: In Vitro Assay Data

In vitro Measurement of Triggering Receptor Expressed on Myeloid Cells 2 activity using cellular phosphorylation of Spleen Tyrosine Kinase (“Syk”) Assays

Measurement of TREM2 agonist potency was done using a HEK cell line expressing human TREM2 and DAP12 (HEK293T-hTREM2 cells). Binding of small molecules to, and activation of, TREM2 increases the phosphorylation of Syk. The resultant levels of Syk phosphorylation are measured using a commercial AlphaLisa reagent kit. To perform the assay, HEK-hTREM2 cells were plated at 14,000 cells per well in a 384 well plate, in 25 μL of complete growth media and incubated at 37° C., 5% CO₂ for 20-24 hours.

Prior to the assay, test compounds were diluted in the 384 well plates in assay buffer and allowed to equilibrate for 30 minutes. Growth media was removed from cell plates by inversion on blotting paper, and 25 μL of test articles in assay buffer was added to cells. Cells were incubated for 45 minutes at room temperature. After 45 minutes, assay buffer was removed and 10 μL of lysis buffer was added. Plates were shaken for 20 minutes at 350 RPM at room temperature. After complete lysis, AlphaLisa reagents were added to the lysate, and fluorescence intensity was measured using a Perkin Elmer Envision plate reader. Intensities were used to generate a standard curve, and % activation was calculated. Curve fitting was performed using Prism v9 software, log (agonist) vs response-variable slope (four parameters), and EC50s were calculated from the curve fit.

The results presented in Table B have been generated with the in vitro assay described above. This assay may be used to test any of the compounds described herein to assess and characterize a compound's ability to act as an agonist of TREM2.

Compounds designated as “A” demonstrated an EC₅₀ of ≤100 nM. Compounds designated as “B” demonstrated an EC₅₀>100 nM and ≤500 nM. Compounds designated as “C” demonstrated an EC₅₀>500 nM.

TABLE B
hTREM2 EC₅₀ Data (HEK293 Cells)

| Ex | hTREM2 |
|---|---|
| # | EC50 nM |
| 1 | A |
| 2 | C |
| 4 | A |
| 5 | A |
| 7 | A |
| 9 | A |
| 11 | B |
| 12 | B |
| 13 | B |
| 14 | A |
| 15 | A |
| 16 | A |
| 18 | B |
| 19 | A |
| 21 | A |
| 23 | A |
| 25 | A |
| 26 | A |
| 27 | A |
| 28 | A |
| 29 | B |
| 31 | A |
| 32 | B |
| 34 | A |
| 36 | A |
| 38 | A |
| 40 | A |
| 42 | A |
| 43 | A |
| 46 | A |
| 48 | A |
| 50 | A |
| 52 | A |
| 54 | A |
| 56 | A |
| 60 | A |
| 62 | A |
| 64 | A |
| 65 | A |
| 66 | A |
| 68 | A |
| 69 | A |
| 71 | A |
| 72 | A |
| 74 | A |
| 75 | A |
| 77 | A |
| 78 | A |
| 80 | A |
| 82 | A |
| 84 | A |
| 86 | A |
| 88 | A |
| 90 | A |
| 92 | C |
| 94 | A |
| 96 | A |
| 98 | A |
| 100 | A |
| 102 | A |
| 104 | A |
| 105 | A |
| 107 | A |
| 109 | A |
| 110 | A |
| 112 | A |
| 114 | A |
| 116 | A |
| 118 | A |
| 120 | A |
| 121 | A |
| 123 | A |
| 124 | B |
| 126 | B |
| 127 | A |
| 129 | A |
| 130 | A |
| 131 | A |
| 132 | A |
| 134 | A |
| 136 | A |
| 138 | A |
| 140 | A |
| 142 | A |
| 145 | A |
| 147 | A |
| 149 | A |
| 153 | A |
| 155 | A |
| 156 | A |
| 158 | A |
| 159 | A |
| 161 | A |
| 162 | A |
| 163 | A |
| 165 | B |
| 167 | A |
| 168 | A |
| 169 | A |
| 170 | B |
| 173 | A |
| 174 | A |
| 175 | A |
| 176 | A |
| 178 | A |
| 179 | A |
| 180 | A |
| 181 | A |
| 182 | A |
| 183 | A |
| 184 | A |
| 185 | B |
| 186 | B |
| 187 | B |
| 188 | C |
| 189 | A |
| 190 | A |
| 191 | A |
| 192 | B |
| 193 | A |
| 194 | B |
| 195 | A |
| 196 | A |
| 197 | B |
| 198 | B |
| 201 | A |
| 202 | B |
| 203 | A |
| 204 | C |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | B |
| 217 | A |
| 218 | B |
| 219 | A |
| 220 | A |
| 324 | A |
| 326 | A |
| 328 | A |
| 330 | A |
| 332 | A |
| 334 | A |
| 336 | A |
| 338 | A |
| 348 | C |
| 349 | C |
| 350 | A |
| 352 | C |
| 353 | A |
| 354 | A |
| 355 | A |

All references, for example, a scientific publication or patent application publication, cited herein are incorporated herein by reference in their entirety and for all purposes to the same extent as if each reference was specifically and individually indicated to be incorporated by reference in its entirety for all purposes.

1. A compound of Formula (I): 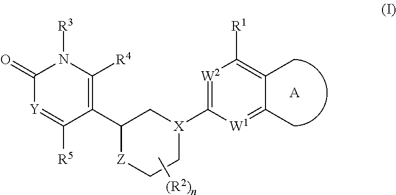
or a pharmaceutically acceptable salt thereof, wherein:
Ring A together with the 6-membered ring system to which it is fused forms a bicyclic ring system selected from one of: 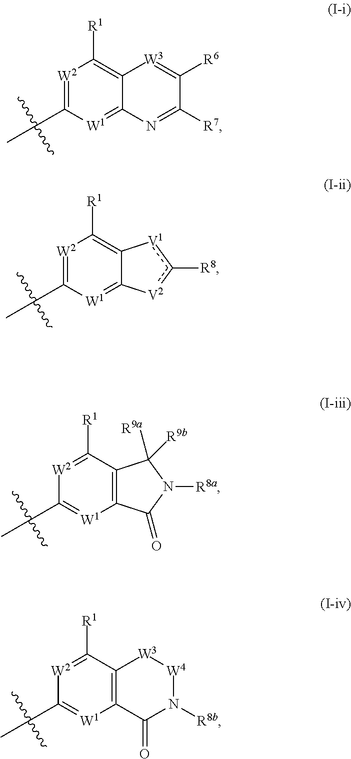
V¹ and V² are each independently N, NR^{8b}, or S, wherein both V¹ and V² are not S, and wherein the bonds in the ring comprising V¹ and V² are either single or double bonds, depending on the valency of V¹ and V²;
W¹, W², W³, and W⁴ are each independently C(R¹⁰), N, or NR^{8b}, wherein the bonds connecting W³ to W⁴ and W⁴ to NR^{8b} are either single or double bonds, depending on the valency of W³ and W⁴;
X is C(R¹¹);
Y is C(R¹²) or N;
Z is C(R¹⁸)₂ or O;
R¹ is cycloalkyl, heterocyclyl, or heteroaryl, each of which is optionally substituted with one or more R¹³;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R¹⁴; or
two R², taken together with the carbon atom to which they are attached, form an oxo group;
R³ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁵;
R⁴ and R⁵ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁴;
R⁶ and R⁷ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁶;
R⁸ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, or N(R^{B})(R^{C}), wherein alkyl, alkenyl, alkynyl, heteroalkyl, haloalkyl, cycloalkyl, and heterocyclyl is optionally substituted with one or more R¹⁶;
R^{8a} is hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶;
R^{8b} is absent, hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶;
R^{9a}, R^{9b}, and R^{9c} are independently in each instance hydrogen, deuterium, C₁₋₆ alkyl, C₂₋₆ alkenyl, C₂₋₆ alkynyl, C₁₋₆ heteroalkyl, C₁₋₆ haloalkyl or halogen;
R¹⁰, R¹¹, and R¹² are each independently in each instance hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁶;
each R¹³ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —C(O)N(R^{B})(R^{C}), or —N(R^{B})(R^{C}), wherein each alkyl, alkenyl, alkynyl, heteroalkyl, haloalkyl, cycloalkyl, and heterocyclyl is optionally substituted with one or more R¹⁷;
each R¹⁴, R¹⁵, and R¹⁶ is independently deuterium, C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁷; or
two R¹⁴, two R¹⁵, or two R¹⁶, together with the carbon atom to which they are attached, form a cycloalkyl, heterocyclyl, aryl, or heteroaryl, wherein each cycloalkyl, heterocyclyl, aryl, and heteroaryl is optionally substituted with one or more R¹⁷;
each R^{A} is independently hydrogen, deuterium, C₁₋₆ alkyl, C₁₋₆ heteroalkyl, C₁₋₆ haloalkyl, cycloalkyl, heterocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₂₋₆ alkenyl, C₁₋₆ heteroalkyl, C₁₋₆ haloalkyl, cycloalkyl, heterocyclyl, or —C(O)-alkyl, wherein each alkyl, alkenyl, heteroalkyl, haloalkyl, cycloalkyl, and heterocyclyl is optionally substituted with one or more R¹⁷; or
R^{B} and R^{C}, together with the nitrogen atom to which they are attached, form —N═C(R^{D})(R^{E}) or a heterocyclyl, wherein the heterocyclyl is optionally substituted with one or more R¹⁷;
each R^{D} and R^{E} is independently hydrogen, C₁₋₆ alkyl, C₂₋₆ alkenyl, C₁₋₆ heteroalkyl, C₁₋₆ haloalkyl, cycloalkyl, or heterocyclyl, wherein each alkyl, alkenyl, heteroalkyl, haloalkyl, cycloalkyl, and heterocyclyl is optionally substituted with one or more R¹⁷; or
each R¹⁷ is deuterium, C₁₋₆ alkyl, C₁₋₆ heteroalkyl, C₁₋₆ haloalkyl, cycloalkyl, halogen, or cyano; or
two R¹⁷ form an oxo;
each R¹⁸ is independently H, C₁₋₆ alkyl, or halo; and
n is 0, 1, 2, 3, 4, 5, or 6.

2. The compound of claim 1, or a pharmaceutically acceptable salt thereof , wherein Ring A is

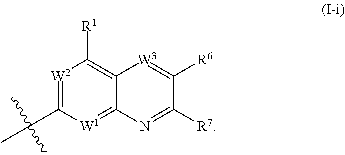

3.-5. (canceled)

6. The compound of claim 2, or a pharmaceutically acceptable salt thereof, wherein W¹, W², and W³ are each N.

7. The compound of claim 1, or a pharmaceutically acceptable salt thereof, wherein R¹ is selected from cycloalkyl or heterocyclyl, each of which is optionally substituted with one or more R¹³.

8. (canceled)

9. The compound of claim 1, or a pharmaceutically acceptable salt thereof, wherein R¹ is a 3-membered, 4-membered, 5-membered, 6-membered, 7-membered, or 8-membered cycloalkyl, each of which is optionally substituted with one or more R¹³.

10. (canceled)

11. The compound of claim 1, or a pharmaceutically acceptable salt thereof, wherein R¹ is selected from

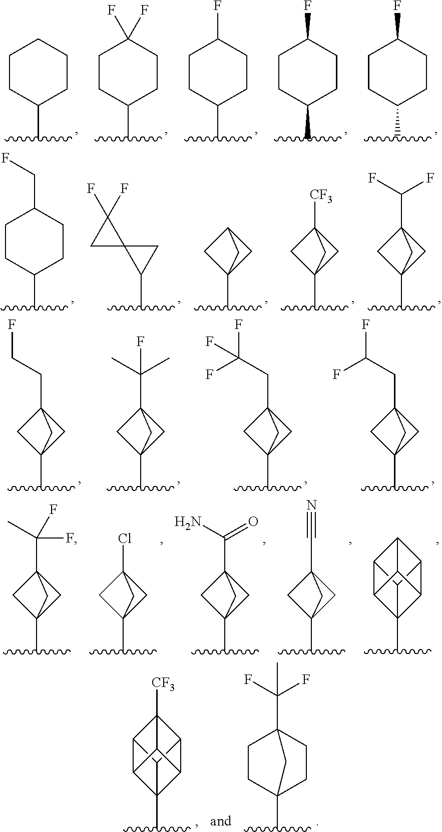

12.-16. (canceled)

17. The compound of claim 1, or a pharmaceutically acceptable salt thereof, wherein R⁶ and R⁷ are each independently hydrogen, C₁₋₆ alkyl, C₁₋₆ heteroalkyl, C₁₋₆ haloalkyl, cycloalkyl, heterocyclyl, aryl, heteroaryl, halogen, cyano, or —O(R^{A}), wherein each alkyl, heteroalkyl, haloalkyl, cycloalkyl, heterocyclyl, aryl, and heteroaryl is optionally substituted with one or more R¹⁶.

18. The compound of claim 1, or a pharmaceutically acceptable salt thereof, wherein R⁶ and R⁷ are each independently C₁₋₆ alkyl.

19.-24. (canceled)

25. The compound of claim 1, or a pharmaceutically acceptable salt thereof, wherein Y is C(R¹²).

26.-30. (canceled)

31. The compound of claim 1, or a pharmaceutically acceptable salt thereof, wherein Z is C(R¹⁸)₂.

32.-33. (canceled)

34. The compound of claim 1, or a pharmaceutically acceptable salt thereof, wherein Z is O.

35. The compound of claim 1, or a pharmaceutically acceptable salt thereof, wherein R³ is hydrogen, C₁₋₆ alkyl, C₁₋₆ heteroalkyl, C₁₋₆ haloalkyl, cycloalkyl, heterocyclyl, aryl, heteroaryl, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁵.

36. The compound of claim 1, or a pharmaceutically acceptable salt thereof, wherein R³ is selected from

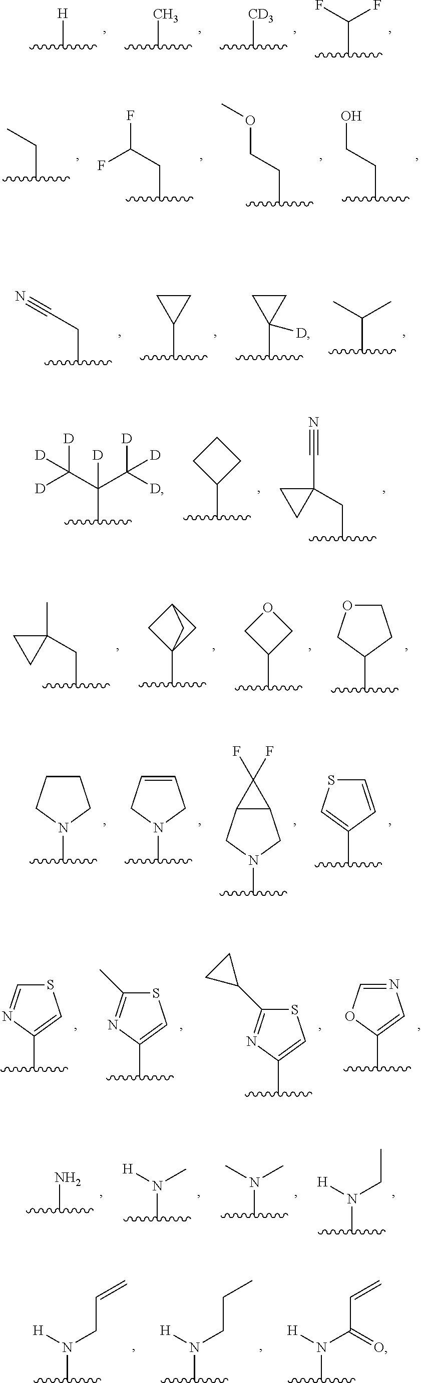

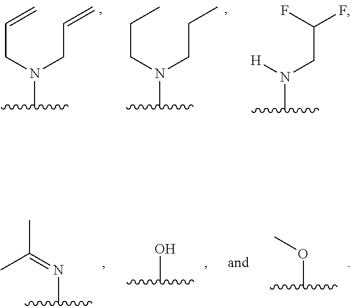

37. The compound of claim 1, or a pharmaceutically acceptable salt thereof, wherein R⁴ and R⁵ are each independently hydrogen or C₁₋₆ alkyl.

38.-40. (canceled)

41. The compound of claim 1, or a pharmaceutically acceptable salt thereof, wherein Ring A is

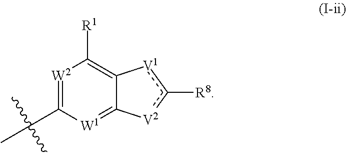

42.-52. (canceled)

53. The compound of claim 1, or a pharmaceutically acceptable salt thereof , wherein Ring A together with the 6-membered ring system to which it is fused forms

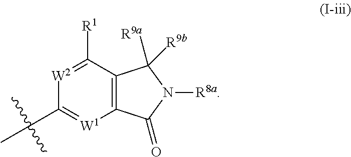

54.-57. (canceled)

58. The compound of claim 1, or a pharmaceutically acceptable salt thereof, wherein Ring A together with the 6-membered ring system to which it is fused is selected from

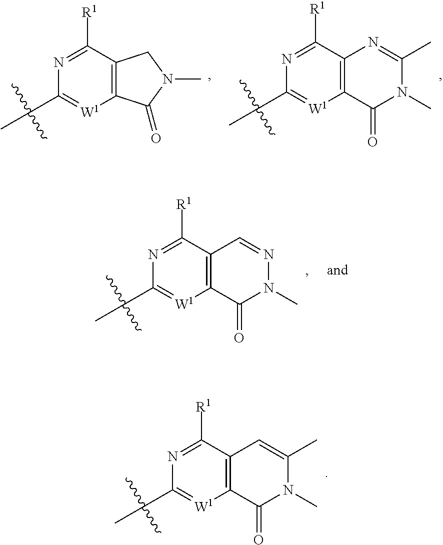

59. The compound of claim 1, wherein the compound of Formula (I) is a compound of Formula (I-a): 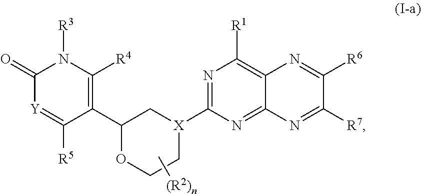
or a pharmaceutically acceptable salt thereof, wherein each of X, R¹, R², R³, R⁴, R⁵, R⁶, R⁷, n and subvariables thereof are defined as for Formula (I).

60. The compound of claim 1, wherein the compound of Formula (I) is a compound of Formula (I-b): 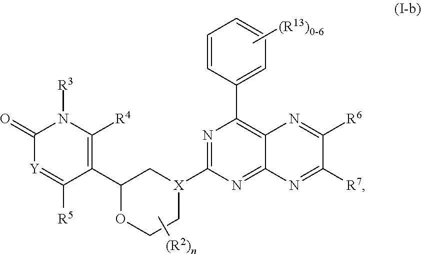
or a pharmaceutically acceptable salt thereof, wherein each of X, R², R³, R⁴, R⁵, R⁶, R⁷, R¹³, n and subvariables thereof are defined as for Formula (I).

61. The compound of claim 1, wherein the compound of Formula (I) is a compound of Formula (I-c): 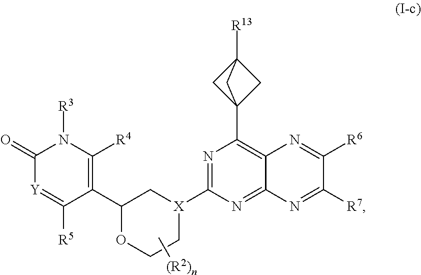
or a pharmaceutically acceptable salt thereof, wherein each of X, R², R³, R⁴, R⁵, R⁶, R⁷, R¹³, n and subvariables thereof are defined as for Formula (I).

62. The compound of claim 1, wherein the compound of Formula (I) is a compound of Formula (I-d): 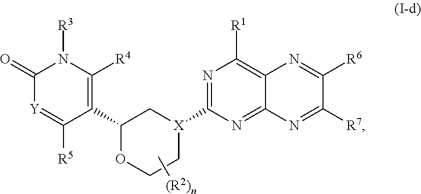
or a pharmaceutically acceptable salt thereof, wherein each of X, R¹, R², R³, R⁴, R⁵, R⁶, R⁷, n and subvariables thereof are defined as for Formula (I).

63. The compound of claim 1, wherein the compound of Formula (I) is a compound of Formula (I-e): 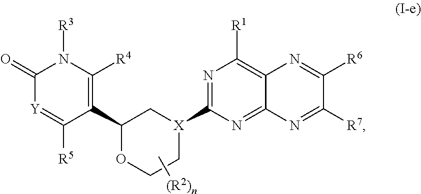
or a pharmaceutically acceptable salt thereof, wherein each of X, R¹, R², R³, R⁴. R⁵, R⁶, R⁷, n and subvariables thereof are defined as for Formula (I).

64.-80. (canceled)

81. The compound of claim 1, wherein the compound of Formula (I) is a compound of Formula (I-x): 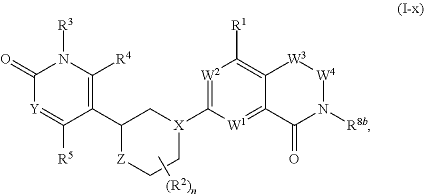
or a pharmaceutically acceptable salt thereof, wherein each of X, Y, Z, R¹, R², R³, R⁴, R⁵, R^{8b}, W¹, W², W³, W⁴, n and subvariables thereof are defined as for Formula (I).

82.-91. (canceled)

92. A pharmaceutical composition comprising the compound according to claim 1, or a pharmaceutically acceptable salt thereof, and a pharmaceutically acceptable excipient.

93. (canceled)

94. A method of treating or preventing a condition associated with a loss of function of human TREM2 in a subject in need thereof, the method comprising administering to the subject in need thereof a therapeutically effective amount of a compound of Formula (I): 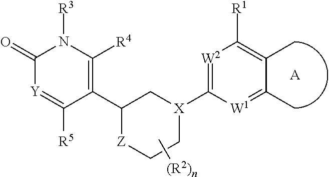
or a pharmaceutically acceptable salt thereof, wherein
Ring A together with the 6-membered ring system to which it is fused forms a bicyclic ring system selected from one of: 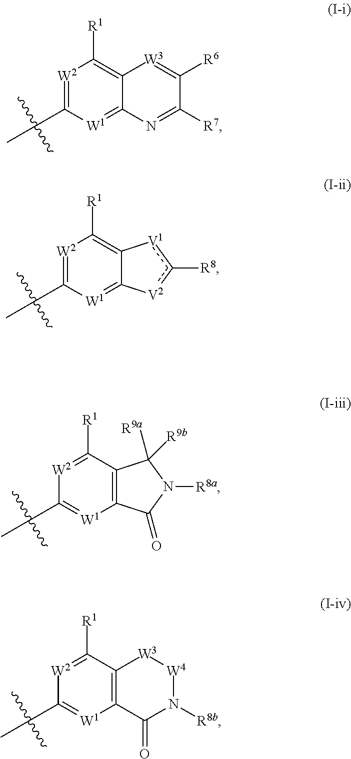
V¹ and V² are each independently N, NR^{8b}, or S, wherein both V¹ and V² are not S, and wherein the bonds in the ring comprising V¹ and V² are either single or double bonds, depending on the valency of V¹ and V²;
W¹, W², W³, and W⁴ are each independently C(R¹⁰), N, or NR^{8b}, wherein the bonds connecting W³ to W⁴ and W⁴ to NR^{8b} are either single or double bonds, depending on the valency of W³ and W⁴:
X is C(R¹¹);
Y is C(R¹²) or N;
Z is C(R¹⁸)₂ or O:
R¹ is cycloalkyl, heterocyclyl, or heteroaryl, each of which is optionally substituted with one or more R¹³;
each R² is independently C₁₋₆ alkyl, C₂₋₆ alkenyl, C₂₋₆ alkynyl, C₁₋₆ heteroalkyl, C₁₋₆ haloalkyl, cycloalkyl, heterocyclyl, halogen, cyano, —O(R^{A}), —N(R^{B})(R^{C}), wherein each alkyl, alkenyl, alkynyl, heteroalkyl, haloalkyl, cycloalkyl, and heterocyclyl is optionally substituted with one or more R¹⁴; or
two R², taken together with the carbon atom to which they are attached, form an oxo group;
R³ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁵;
R⁴ and R⁵ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁴;
R⁶ and R⁷ are each independently hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein alkyl, alkenyl, alkynyl, heteroalkyl, haloalkyl, cycloalkyl, heterocyclyl, aryl, and heteroaryl is optionally substituted with one or more R¹⁶;
R⁸ is hydrogen, C₁₋₆ alkyl, C₂₋₆ alkenyl, C₂₋₆ alkynyl, C₁₋₆ heteroalkyl, C₁₋₆ haloalkyl, cycloalkyl, heterocyclyl, or N(R^{B})(R^{C}), wherein alkyl, alkenyl, alkynyl, heteroalkyl, haloalkyl, cycloalkyl, and heterocyclyl is optionally substituted with one or more R¹⁶,
R^{8a} is hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶,
R^{8b} is absent, hydrogen, C₁₋₆ alkyl, C₂₋₆ alkenyl, C₂₋₆ alkynyl, C₁₋₆ heteroalkyl, C₁₋₆ haloalkyl, cycloalkyl, or heterocyclyl, wherein alkyl, alkenyl, alkynyl, heteroalkyl, haloalkyl, cycloalkyl, and heterocyclyl is optionally substituted with one or more R¹⁶;
R^{9a}, R^{9b}, and R^{9c} are independently in each instance hydrogen, deuterium, C₁₋₆ alkyl, C₂₋₆ alkenyl, C₂₋₆ alkynyl, C₁₋₆ heteroalkyl, C₁₋₆ haloalkyl or halogen;
R¹⁰, R¹¹, and R¹² are each independently in each instance hydrogen, C₁₆ alkyl, C₂₋₆ alkenyl, C₂₋₆ alkynyl, C₁₋₆ heteroalkyl, C₁₋₆ haloalkyl, cycloalkyl, heterocyclyl, aryl, heteroaryl, halogen, cyano, —O(R^{A}), or —N(R^{B})(R^{C}), wherein each alkyl, alkenyl, alkynyl, heteroalkyl, haloalkyl, cycloalkyl, heterocyclyl, aryl, and heteroaryl is optionally substituted with one or more R¹⁶;
each R¹³ is independently deuterium, C₁₋₆ alkyl, C₂₋₆ alkenyl, C₂₆ alkynyl, C₁₋₆ heteroalkyl, C₁₋₆ haloalkyl, cycloalkyl, heterocyclyl, halogen, cyano, —O(R^{A}), —C(O)N(R^{B})(R^{C}), or —N(R^{B})(R^{C}), wherein each alkyl, alkenyl, alkynyl, heteroalkyl, haloalkyl, cycloalkyl, and heterocyclyl is optionally substituted with one or more R¹⁷;
each R¹⁴, R¹⁵, and R¹⁶ is independently deuterium, C₁₋₆ alkyl, C₂₋₆ alkenyl, C₂₋₆ alkynyl, C₁-6 heteroalkyl, C₁₋₆ haloalkyl, cycloalkyl, heterocyclyl, halogen, cyano, —O(R^{A}), or —N(R^{B})(R^{C}), wherein each alkyl, alkenyl, alkynyl, heteroalkyl, haloalkyl, cycloalkyl, and heterocyclyl is optionally substituted with one or more R¹⁷; or
two R¹⁴, two R¹⁵, or two R¹⁶, together with the carbon atom to which they are attached, form a cycloalkyl, heterocyclyl, aryl, or heteroaryl, wherein each cycloalkyl, heterocyclyl, aryl and heteroaryl is optionally substituted with one or more R¹⁷;
each R^{A} is independently hydrogen, deuterium, C₁₋₆ alkyl, C₁₋₆ heteroalkyl, C₁₋₆ haloalkyl, cycloalkyl, heteerocyclyl, or —N(R^{B})(R^{C}), wherein each alkyl, heteroalkyl, haloalkyl, cycloalkyl, and heterocyclyl is optionally substituted with one or more R¹⁷;
each R^{B} and R^{C} is independently hydrogen, C₁₋₆ alkyl, C₂₋₆ alkenyl, C₁₋₆ heteroalkyl, C₁₋₆ haloalkyl, cycloalkyl, heterocyclyl, or —C(O)-alkyl, wherein each alkyl, alkenyl, heteroalkyl, haloalkyl, cycloalkyl, and heterocyclyl is optionally substituted with one or more R¹⁷, or
R^{B} and R^{C}, together with the nitrogen atom to which they are attached, form —N═C(R^{D})(R^{E}) or a heterocyclyl, wherein the heterocyclyl is optionally substituted with one or more R¹⁷;
each R^{D} and R^{E} is independently hydrogen, C₁₋₆ alkyl, C₂₆ alkenyl, C₁₋₆ heteroalkyl, C₁₋₆ haloalkyl, cycloalkyl, or heterocyclyl, wherein each alkyl, alkenyl, heteroalkyl, haloalkyl, cycloalkyl, and heterocyclyl is optionally substituted with one or more R¹⁷; or
each R¹⁷ is deuterium, C₁₋₆ alkyl, C₁₋₆ heteroalkyl, C₁₋₆ haloalkyl, cycloalkyl, halogen, or cyano; or
two R¹⁷ form an oxo;
each R¹⁸ is independently H, C₁₋₆ alkyl, or halo; and
n is 0, 1, 2, 3, 4, 5, or 6.

95. The method of claim 94, wherein the condition is selected from the group consisting of proliferative diseases, cardiovascular diseases, metabolic diseases, inflammatory diseases, autoimmune disorders, neurodegenerative disorders, infectious diseases, and tissue injuries.

96. The method of claim 94, wherein the condition is selected from the group consisting of dementia, Alzheimer's disease, Creutzfeldt-Jakob disease, Parkinson's disease, dementia with Lewy bodies, amyotrophic lateral sclerosis (ALS), Huntington's disease, taupathy disease, Nasu-Hakola disease, dry age-related macular degeneration (dry AMD), multiple system atrophy (MSA), Shy-Drager syndrome, progressive supranuclear palsy, cortical basal ganglionic degeneration, glaucoma, retinitis pigmentosa, and retinal degeneration.

97. The compound of claim 1, wherein the compound is selected from the group consisting of the following compounds of Table A, or a pharmaceutically acceptable salt thereof:

| Ex | |
|---|---|
| # | Structure |
| 1 | 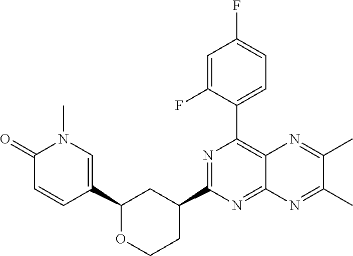 [SMILES: Cc1nc2nc([C@H]3CCO[C@@H](c4ccc(=O)n(C)c4)C3)nc(-c3ccc(F)cc3F)c2nc1C] |
| 2 | 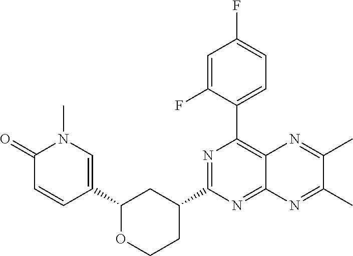 [SMILES: Cc1nc2nc([C@@H]3CCO[C@H](c4ccc(=O)n(C)c4)C3)nc(-c3ccc(F)cc3F)c2nc1C] |
| 3 | 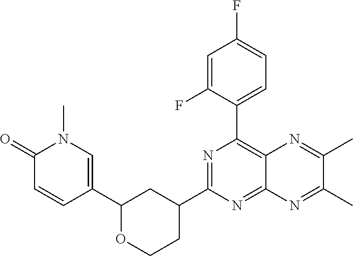 [SMILES: Cc1nc2nc(C3CCOC(c4ccc(=O)n(C)c4)C3)nc(-c3ccc(F)cc3F)c2nc1C] |
| 4 | 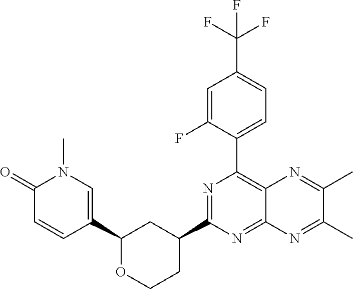 [SMILES: Cc1nc2nc([C@H]3CCO[C@@H](c4ccc(=O)n(C)c4)C3)nc(-c3ccc(C(F)(F)F)cc3F)c2nc1C] |
| 5 | 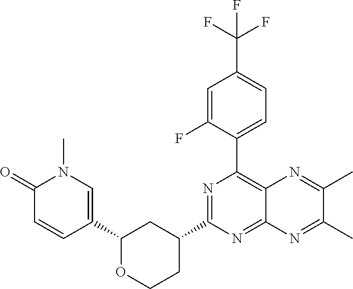 [SMILES: Cc1nc2nc([C@@H]3CCO[C@H](c4ccc(=O)n(C)c4)C3)nc(-c3ccc(C(F)(F)F)cc3F)c2nc1C] |
| 6 | 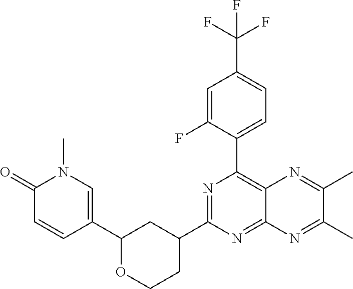 [SMILES: Cc1nc2nc(C3CCOC(c4ccc(=O)n(C)c4)C3)nc(-c3ccc(C(F)(F)F)cc3F)c2nc1C] |
| 7 | 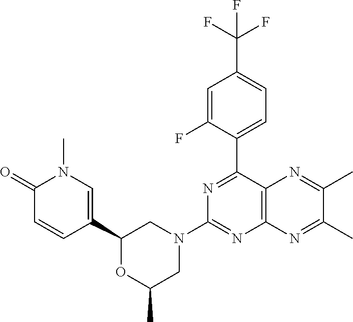 [SMILES: Cc1nc2nc(N3C[C@@H](C)O[C@@H](c4ccc(=O)n(C)c4)C3)nc(-c3ccc(C(F)(F)F)cc3F)c2nc1C] |
| 9 | 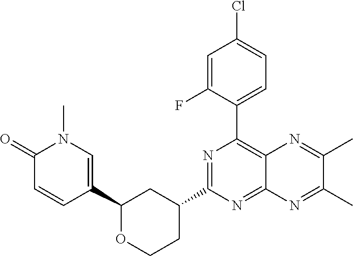 [SMILES: Cc1nc2nc([C@@H]3CCO[C@@H](c4ccc(=O)n(C)c4)C3)nc(-c3ccc(Cl)cc3F)c2nc1C] |
| 10 | 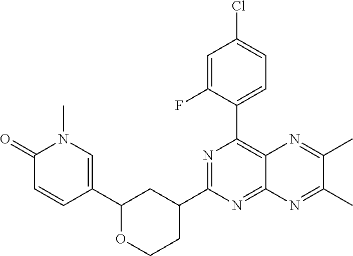 [SMILES: Cc1nc2nc(C3CCOC(c4ccc(=O)n(C)c4)C3)nc(-c3ccc(Cl)cc3F)c2nc1C] |
| 11 | 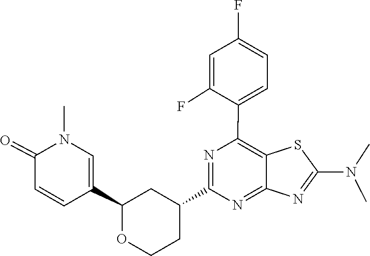 [SMILES: CN(C)c1nc2nc([C@@H]3CCO[C@@H](c4ccc(=O)n(C)c4)C3)nc(-c3ccc(F)cc3F)c2s1] |
| 12 | 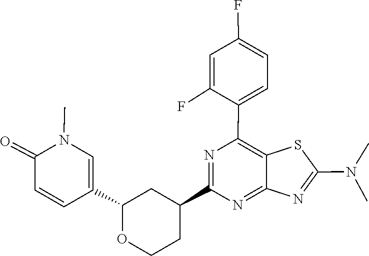 [SMILES: CN(C)c1nc2nc([C@H]3CCO[C@H](c4ccc(=O)n(C)c4)C3)nc(-c3ccc(F)cc3F)c2s1] |
| 13 | 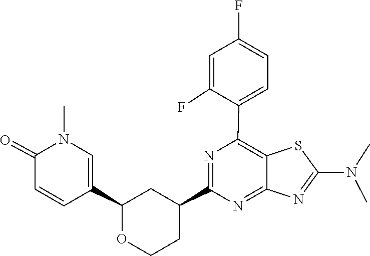 [SMILES: CN(C)c1nc2nc([C@H]3CCO[C@@H](c4ccc(=O)n(C)c4)C3)nc(-c3ccc(F)cc3F)c2s1] |
| 14 | 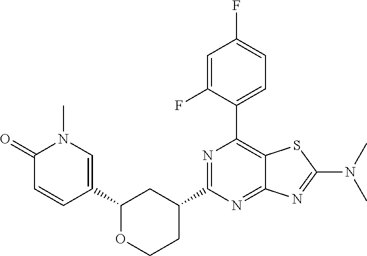 [SMILES: CN(C)c1nc2nc([C@@H]3CCO[C@H](c4ccc(=O)n(C)c4)C3)nc(-c3ccc(F)cc3F)c2s1] |
| 15 | 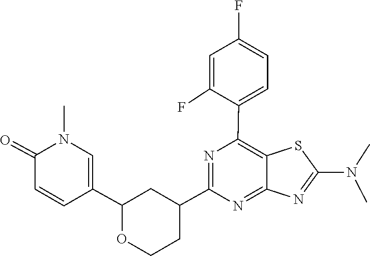 [SMILES: CN(C)c1nc2nc(C3CCOC(c4ccc(=O)n(C)c4)C3)nc(-c3ccc(F)cc3F)c2s1] |
| 18 | 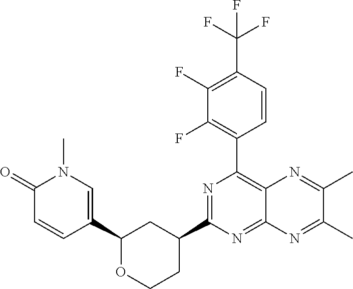 [SMILES: Cc1nc2nc([C@H]3CCO[C@@H](c4ccc(=O)n(C)c4)C3)nc(-c3ccc(C(F)(F)F)c(F)c3F)c2nc1C] |
| 19 | 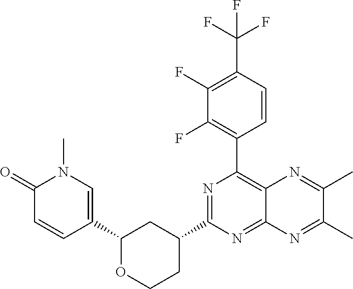 [SMILES: Cc1nc2nc([C@@H]3CCO[C@H](c4ccc(=O)n(C)c4)C3)nc(-c3ccc(C(F)(F)F)c(F)c3F)c2nc1C] |
| 20 | 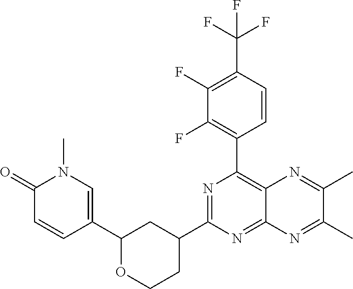 [SMILES: Cc1nc2nc(C3CCOC(c4ccc(=O)n(C)c4)C3)nc(-c3ccc(C(F)(F)F)c(F)c3F)c2nc1C] |
| 25 | 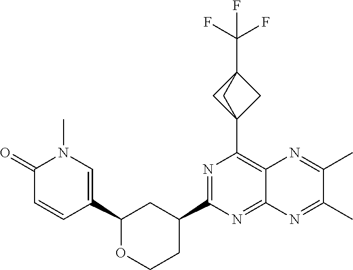 [SMILES: Cc1nc2nc([C@H]3CCO[C@@H](c4ccc(=O)n(C)c4)C3)nc(C34CC(C(F)(F)F)(C3)C4)c2nc1C] |
| 26 | 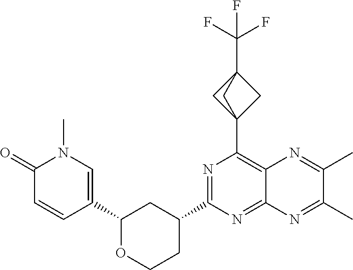 [SMILES: Cc1nc2nc([C@@H]3CCO[C@H](c4ccc(=O)n(C)c4)C3)nc(C34CC(C(F)(F)F)(C3)C4)c2nc1C] |
| 27 | 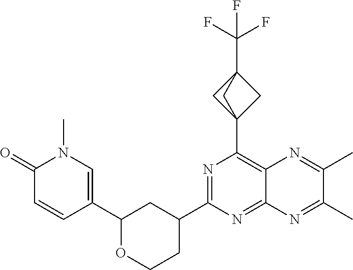 [SMILES: Cc1nc2nc(C3CCOC(c4ccc(=O)n(C)c4)C3)nc(C34CC(C(F)(F)F)(C3)C4)c2nc1C] |
| 28 | 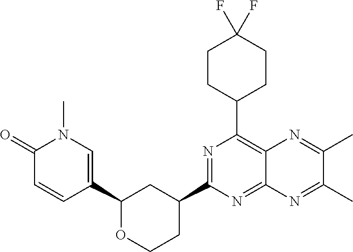 [SMILES: Cc1nc2nc([C@H]3CCO[C@@H](c4ccc(=O)n(C)c4)C3)nc(C3CCC(F)(F)CC3)c2nc1C] |
| 29 | 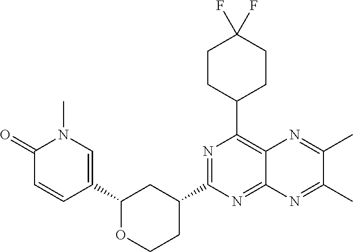 [SMILES: Cc1nc2nc([C@@H]3CCO[C@H](c4ccc(=O)n(C)c4)C3)nc(C3CCC(F)(F)CC3)c2nc1C] |
| 30 | 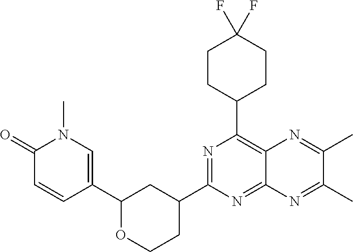 [SMILES: Cc1nc2nc(C3CCOC(c4ccc(=O)n(C)c4)C3)nc(C3CCC(F)(F)CC3)c2nc1C] |
| 31 | 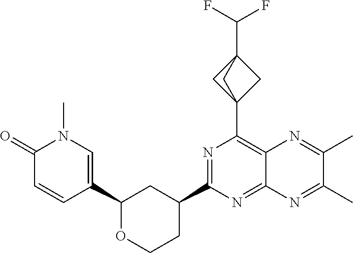 [SMILES: Cc1nc2nc([C@H]3CCO[C@@H](c4ccc(=O)n(C)c4)C3)nc(C34CC(C(F)F)(C3)C4)c2nc1C] |
| 32 | 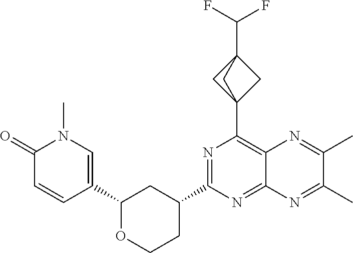 [SMILES: Cc1nc2nc([C@@H]3CCO[C@H](c4ccc(=O)n(C)c4)C3)nc(C34CC(C(F)F)(C3)C4)c2nc1C] |
| 33 | 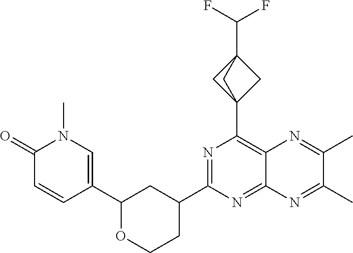 [SMILES: Cc1nc2nc(C3CCOC(c4ccc(=O)n(C)c4)C3)nc(C34CC(C(F)F)(C3)C4)c2nc1C] |
| 34 | 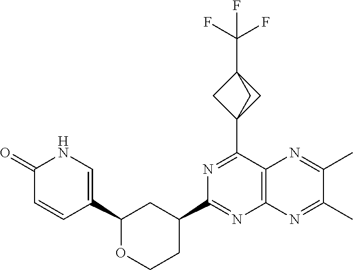 [SMILES: Cc1nc2nc([C@H]3CCO[C@@H](c4ccc(=O)[nH]c4)C3)nc(C34CC(C(F)(F)F)(C3)C4)c2nc1C] |
| 35 | 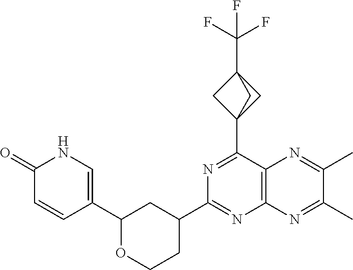 [SMILES: Cc1nc2nc(C3CCOC(c4ccc(=O)[nH]c4)C3)nc(C34CC(C(F)(F)F)(C3)C4)c2nc1C] |
| 36 | 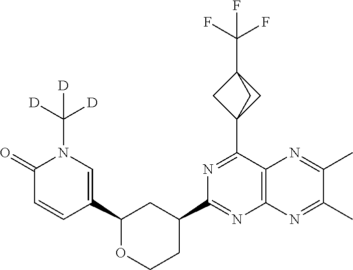 [SMILES: [2H]C([2H])([2H])n1cc([C@H]2C[C@@H](c3nc(C45CC(C(F)(F)F)(C4)C5)c4nc(C)c(C)nc4n3)CCO2)ccc1=O] |
| 37 | 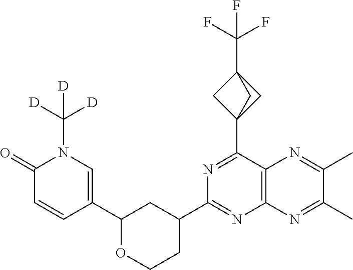 [SMILES: [2H]C([2H])([2H])n1cc(C2CC(c3nc(C45CC(C(F)(F)F)(C4)C5)c4nc(C)c(C)nc4n3)CCO2)ccc1=O] |
| 38 | 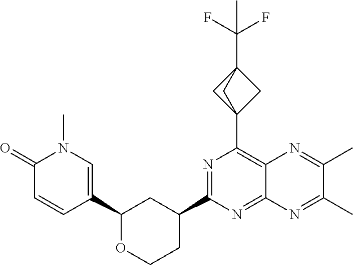 [SMILES: Cc1nc2nc([C@H]3CCO[C@@H](c4ccc(=O)n(C)c4)C3)nc(C34CC(C(C)(F)F)(C3)C4)c2nc1C] |
| 39 | 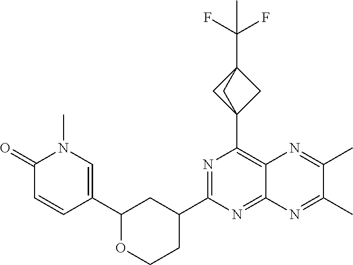 [SMILES: Cc1nc2nc(C3CCOC(c4ccc(=O)n(C)c4)C3)nc(C34CC(C(C)(F)F)(C3)C4)c2nc1C] |
| 40 | 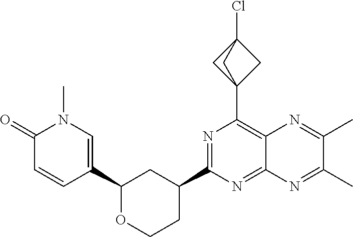 [SMILES: Cc1nc2nc([C@H]3CCO[C@@H](c4ccc(=O)n(C)c4)C3)nc(C34CC(Cl)(C3)C4)c2nc1C] |
| 41 | 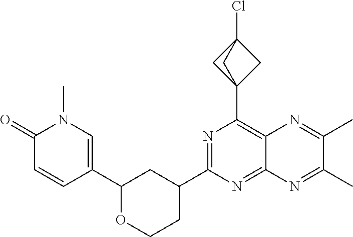 [SMILES: Cc1nc2nc(C3CCOC(c4ccc(=O)n(C)c4)C3)nc(C34CC(Cl)(C3)C4)c2nc1C] |
| 42 | 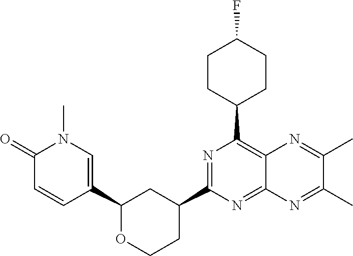 [SMILES: Cc1nc2nc([C@H]3CCO[C@@H](c4ccc(=O)n(C)c4)C3)nc([C@H]3CC[C@H](F)CC3)c2nc1C] |
| 43 | 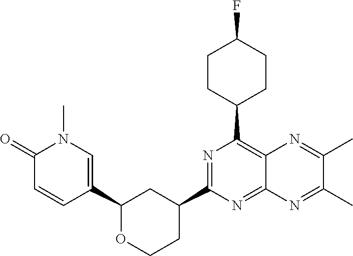 [SMILES: Cc1nc2nc([C@H]3CCO[C@@H](c4ccc(=O)n(C)c4)C3)nc([C@H]3CC[C@@H](F)CC3)c2nc1C] |
| 44 | 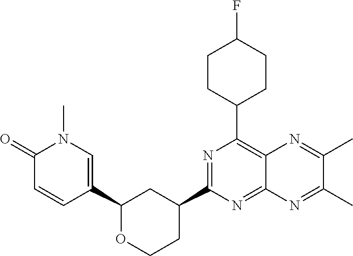 [SMILES: Cc1nc2nc([C@H]3CCO[C@@H](c4ccc(=O)n(C)c4)C3)nc(C3CCC(F)CC3)c2nc1C] |
| 45 | 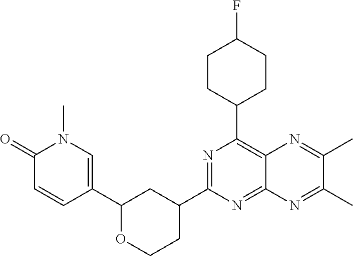 [SMILES: Cc1nc2nc(C3CCOC(c4ccc(=O)n(C)c4)C3)nc(C3CCC(F)CC3)c2nc1C] |
| 46 | 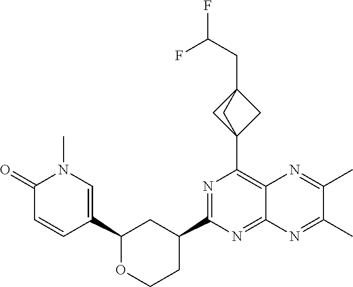 [SMILES: Cc1nc2nc([C@H]3CCO[C@@H](c4ccc(=O)n(C)c4)C3)nc(C34CC(CC(F)F)(C3)C4)c2nc1C] |
| 47 | 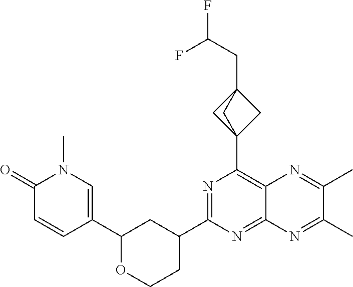 [SMILES: Cc1nc2nc(C3CCOC(c4ccc(=O)n(C)c4)C3)nc(C34CC(CC(F)F)(C3)C4)c2nc1C] |
| 48 | 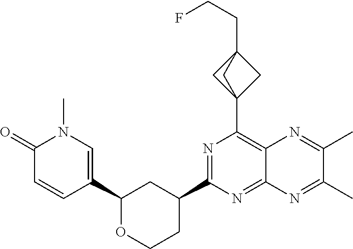 [SMILES: Cc1nc2nc([C@H]3CCO[C@@H](c4ccc(=O)n(C)c4)C3)nc(C34CC(CCF)(C3)C4)c2nc1C] |
| 49 | 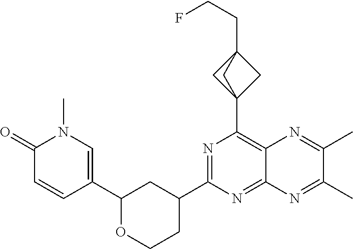 [SMILES: Cc1nc2nc(C3CCOC(c4ccc(=O)n(C)c4)C3)nc(C34CC(CCF)(C3)C4)c2nc1C] |
| 50 | 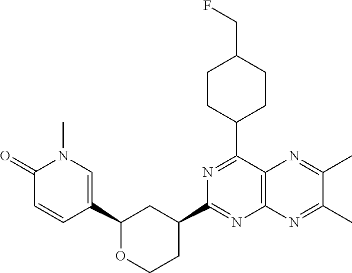 [SMILES: Cc1nc2nc([C@H]3CCO[C@@H](c4ccc(=O)n(C)c4)C3)nc(C3CCC(CF)CC3)c2nc1C] |
| 51 | 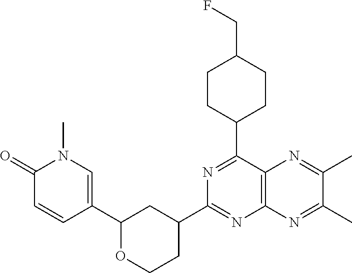 [SMILES: Cc1nc2nc(C3CCOC(c4ccc(=O)n(C)c4)C3)nc(C3CCC(CF)CC3)c2nc1C] |
| 52 | 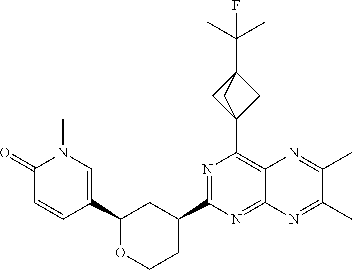 [SMILES: Cc1nc2nc([C@H]3CCO[C@@H](c4ccc(=O)n(C)c4)C3)nc(C34CC(C(C)(C)F)(C3)C4)c2nc1C] |
| 53 | 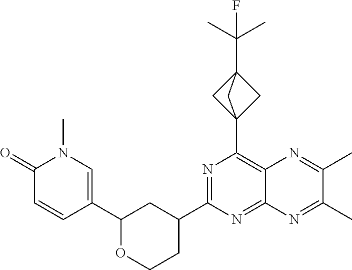 [SMILES: Cc1nc2nc(C3CCOC(c4ccc(=O)n(C)c4)C3)nc(C34CC(C(C)(C)F)(C3)C4)c2nc1C] |
| 54 | 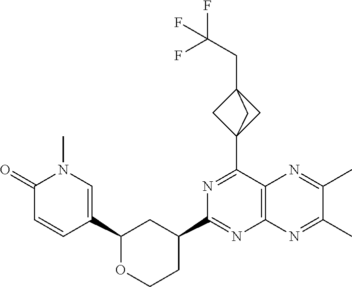 [SMILES: Cc1nc2nc([C@H]3CCO[C@@H](c4ccc(=O)n(C)c4)C3)nc(C34CC(CC(F)(F)F)(C3)C4)c2nc1C] |
| 55 | 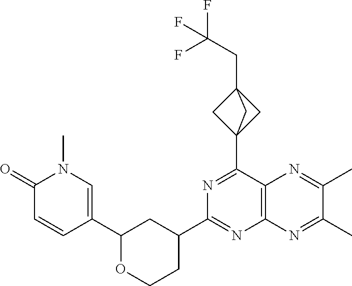 [SMILES: Cc1nc2nc(C3CCOC(c4ccc(=O)n(C)c4)C3)nc(C34CC(CC(F)(F)F)(C3)C4)c2nc1C] |
| 56 | 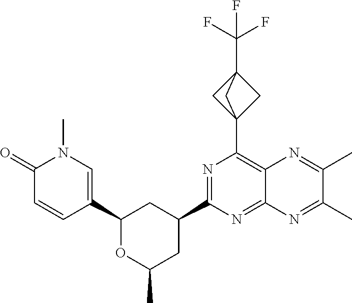 [SMILES: Cc1nc2nc([C@H]3C[C@@H](C)O[C@@H](c4ccc(=O)n(C)c4)C3)nc(C34CC(C(F)(F)F)(C3)C4)c2nc1C] |
| 57 | 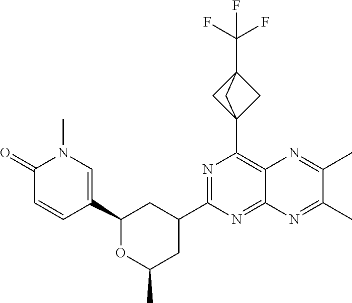 [SMILES: Cc1nc2nc(C3C[C@@H](C)O[C@@H](c4ccc(=O)n(C)c4)C3)nc(C34CC(C(F)(F)F)(C3)C4)c2nc1C] |
| 58 | 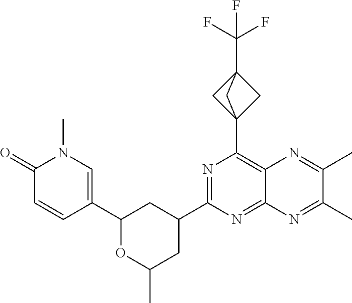 [SMILES: Cc1nc2nc(C3CC(C)OC(c4ccc(=O)n(C)c4)C3)nc(C34CC(C(F)(F)F)(C3)C4)c2nc1C] |
| 59 | 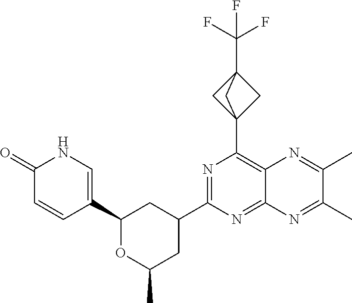 [SMILES: Cc1nc2nc(C3C[C@@H](C)O[C@@H](c4ccc(=O)[nH]c4)C3)nc(C34CC(C(F)(F)F)(C3)C4)c2nc1C] |
| 60 | 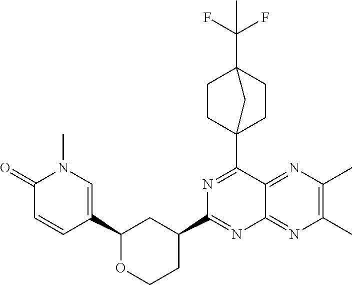 [SMILES: Cc1nc2nc([C@H]3CCO[C@@H](c4ccc(=O)n(C)c4)C3)nc(C34CCC(C(C)(F)F)(CC3)C4)c2nc1C] |
| 61 | 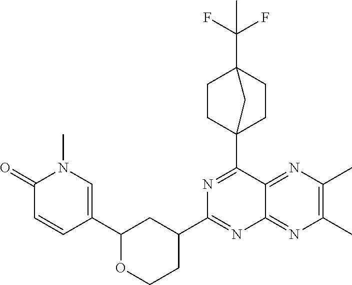 [SMILES: Cc1nc2nc(C3CCOC(c4ccc(=O)n(C)c4)C3)nc(C34CCC(C(C)(F)F)(CC3)C4)c2nc1C] |
| 64 | 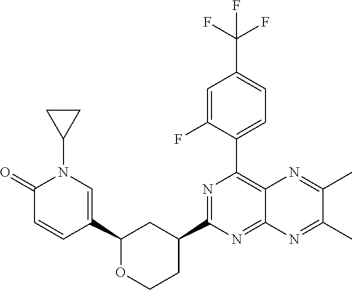 [SMILES: Cc1nc2nc([C@H]3CCO[C@@H](c4ccc(=O)n(C5CC5)c4)C3)nc(-c3ccc(C(F)(F)F)cc3F)c2nc1C] |
| 65 | 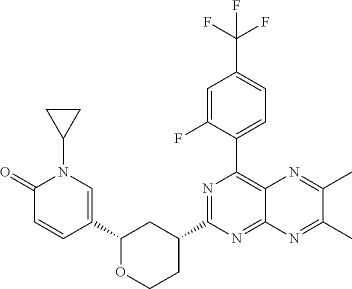 [SMILES: Cc1nc2nc([C@@H]3CCO[C@H](c4ccc(=O)n(C5CC5)c4)C3)nc(-c3ccc(C(F)(F)F)cc3F)c2nc1C] |
| 66 | 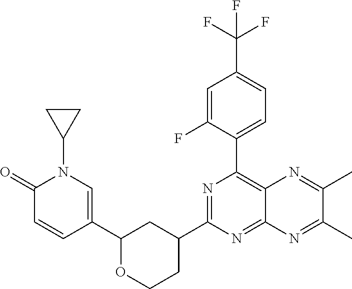 [SMILES: Cc1nc2nc(C3CCOC(c4ccc(=O)n(C5CC5)c4)C3)nc(-c3ccc(C(F)(F)F)cc3F)c2nc1C] |
| 67 | 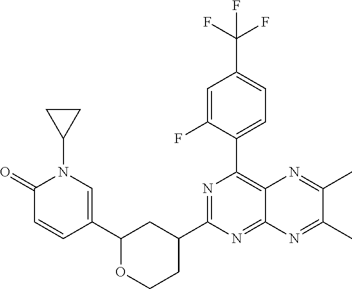 [SMILES: Cc1nc2nc(C3CCOC(c4ccc(=O)n(C5CC5)c4)C3)nc(-c3ccc(C(F)(F)F)cc3F)c2nc1C] |
| 68 | 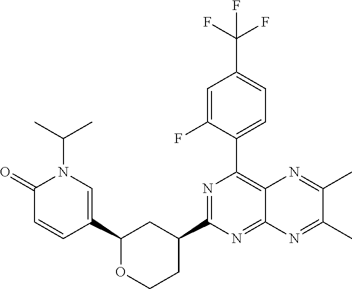 [SMILES: Cc1nc2nc([C@H]3CCO[C@@H](c4ccc(=O)n(C(C)C)c4)C3)nc(-c3ccc(C(F)(F)F)cc3F)c2nc1C] |
| 69 | 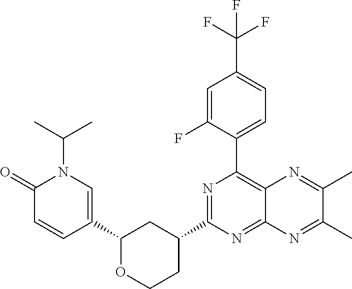 [SMILES: Cc1nc2nc([C@@H]3CCO[C@H](c4ccc(=O)n(C(C)C)c4)C3)nc(-c3ccc(C(F)(F)F)cc3F)c2nc1C] |
| 70 | 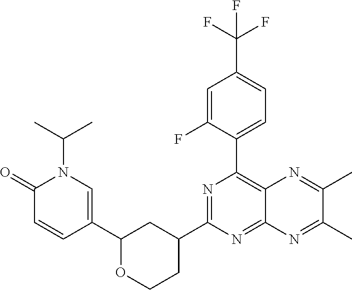 [SMILES: Cc1nc2nc(C3CCOC(c4ccc(=O)n(C(C)C)c4)C3)nc(-c3ccc(C(F)(F)F)cc3F)c2nc1C] |
| 71 | 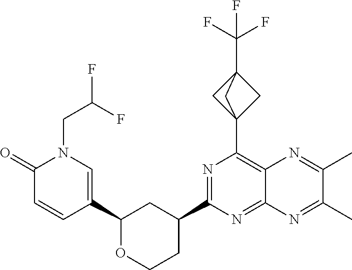 [SMILES: Cc1nc2nc([C@H]3CCO[C@@H](c4ccc(=O)n(CC(F)F)c4)C3)nc(C34CC(C(F)(F)F)(C3)C4)c2nc1C] |
| 72 | 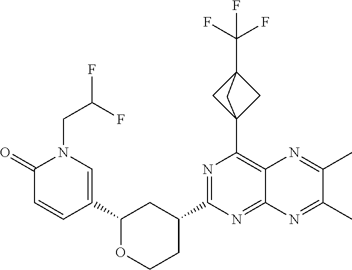 [SMILES: Cc1nc2nc([C@@H]3CCO[C@H](c4ccc(=O)n(CC(F)F)c4)C3)nc(C34CC(C(F)(F)F)(C3)C4)c2nc1C] |
| 73 | 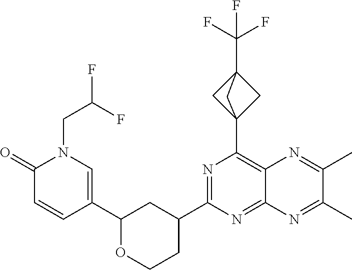 [SMILES: Cc1nc2nc(C3CCOC(c4ccc(=O)n(CC(F)F)c4)C3)nc(C34CC(C(F)(F)F)(C3)C4)c2nc1C] |
| 74 | 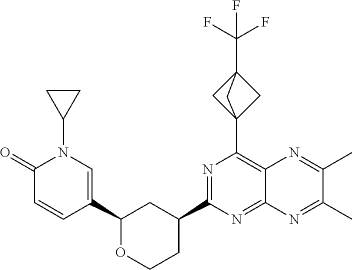 [SMILES: Cc1nc2nc([C@H]3CCO[C@@H](c4ccc(=O)n(C5CC5)c4)C3)nc(C34CC(C(F)(F)F)(C3)C4)c2nc1C] |
| 75 | 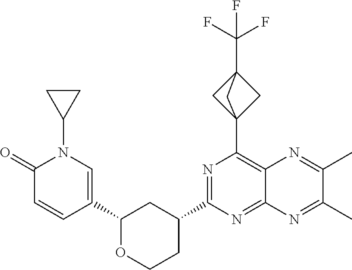 [SMILES: Cc1nc2nc([C@@H]3CCO[C@H](c4ccc(=O)n(C5CC5)c4)C3)nc(C34CC(C(F)(F)F)(C3)C4)c2nc1C] |
| 76 | 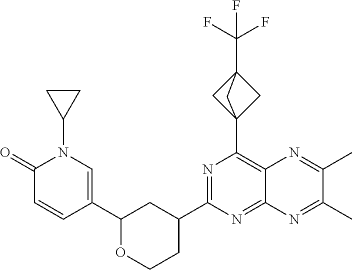 [SMILES: Cc1nc2nc(C3CCOC(c4ccc(=O)n(C5CC5)c4)C3)nc(C34CC(C(F)(F)F)(C3)C4)c2nc1C] |
| 77 | 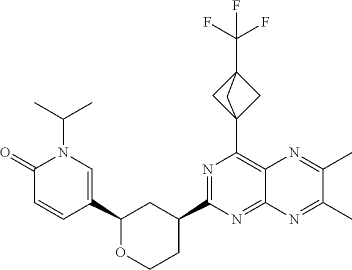 [SMILES: Cc1nc2nc([C@H]3CCO[C@@H](c4ccc(=O)n(C(C)C)c4)C3)nc(C34CC(C(F)(F)F)(C3)C4)c2nc1C] |
| 78 | 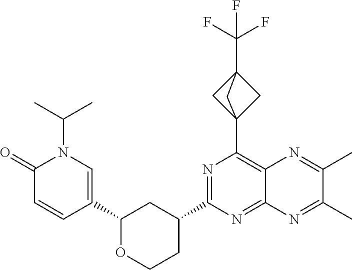 [SMILES: Cc1nc2nc([C@@H]3CCO[C@H](c4ccc(=O)n(C(C)C)c4)C3)nc(C34CC(C(F)(F)F)(C3)C4)c2nc1C] |
| 79 | 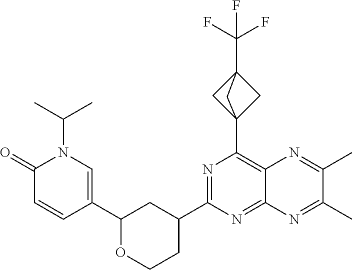 [SMILES: Cc1nc2nc(C3CCOC(c4ccc(=O)n(C(C)C)c4)C3)nc(C34CC(C(F)(F)F)(C3)C4)c2nc1C] |
| 80 | 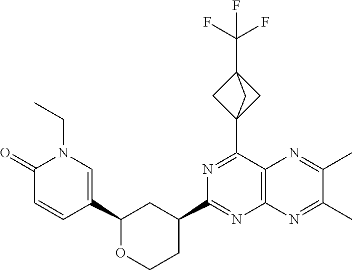 [SMILES: CCn1cc([C@H]2C[C@@H](c3nc(C45CC(C(F)(F)F)(C4)C5)c4nc(C)c(C)nc4n3)CCO2)ccc1=O] |
| 81 | 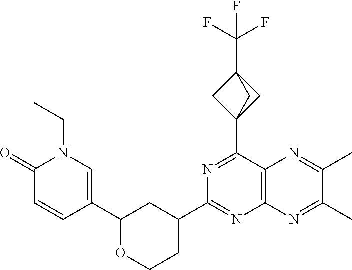 [SMILES: CCn1cc(C2CC(c3nc(C45CC(C(F)(F)F)(C4)C5)c4nc(C)c(C)nc4n3)CCO2)ccc1=O] |
| 82 | 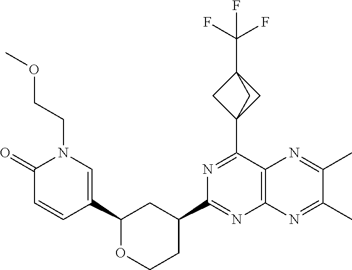 [SMILES: COCCn1cc([C@H]2C[C@@H](c3nc(C45CC(C(F)(F)F)(C4)C5)c4nc(C)c(C)nc4n3)CCO2)ccc1=O] |
| 83 | 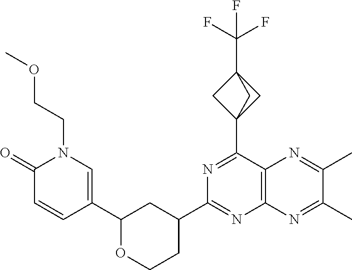 [SMILES: COCCn1cc(C2CC(c3nc(C45CC(C(F)(F)F)(C4)C5)c4nc(C)c(C)nc4n3)CCO2)ccc1=O] |
| 84 | 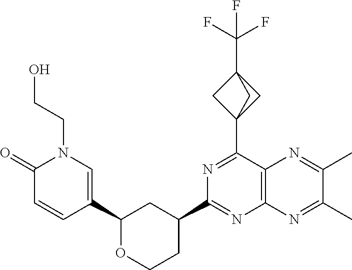 [SMILES: Cc1nc2nc([C@H]3CCO[C@@H](c4ccc(=O)n(CCO)c4)C3)nc(C34CC(C(F)(F)F)(C3)C4)c2nc1C] |
| 85 | 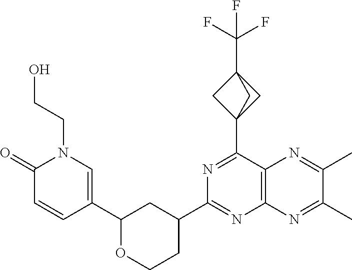 [SMILES: Cc1nc2nc(C3CCOC(c4ccc(=O)n(CCO)c4)C3)nc(C34CC(C(F)(F)F)(C3)C4)c2nc1C] |
| 86 | 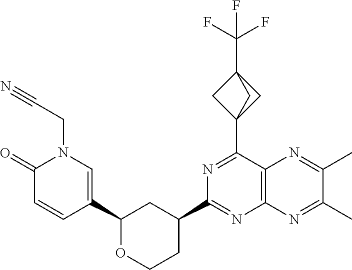 [SMILES: Cc1nc2nc([C@H]3CCO[C@@H](c4ccc(=O)n(CC#N)c4)C3)nc(C34CC(C(F)(F)F)(C3)C4)c2nc1C] |
| 87 | 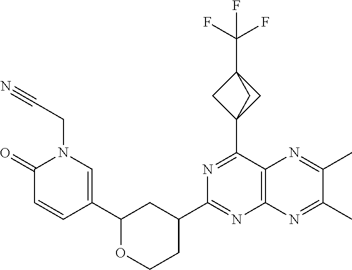 [SMILES: Cc1nc2nc(C3CCOC(c4ccc(=O)n(CC#N)c4)C3)nc(C34CC(C(F)(F)F)(C3)C4)c2nc1C] |
| 88 | 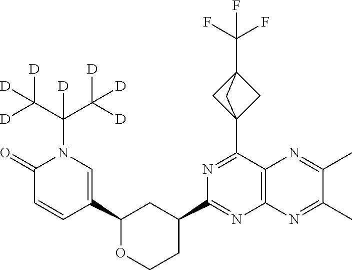 [SMILES: [2H]C([2H])([2H])C([2H])(n1cc([C@H]2C[C@@H](c3nc(C45CC(C(F)(F)F)(C4)C5)c4nc(C)c(C)nc4n3)CCO2)ccc1=O)C([2H])([2H])[2H]] |
| 89 | 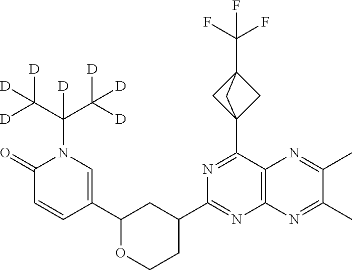 [SMILES: [2H]C([2H])([2H])C([2H])(n1cc(C2CC(c3nc(C45CC(C(F)(F)F)(C4)C5)c4nc(C)c(C)nc4n3)CCO2)ccc1=O)C([2H])([2H])[2H]] |
| 90 | 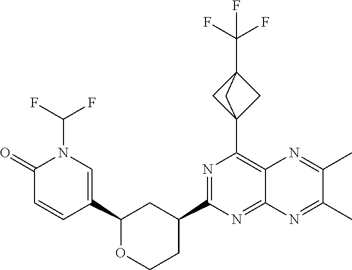 [SMILES: Cc1nc2nc([C@H]3CCO[C@@H](c4ccc(=O)n(C(F)F)c4)C3)nc(C34CC(C(F)(F)F)(C3)C4)c2nc1C] |
| 91 | 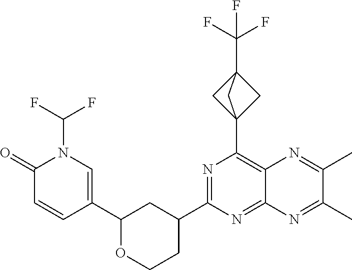 [SMILES: Cc1nc2nc(C3CCOC(c4ccc(=O)n(C(F)F)c4)C3)nc(C34CC(C(F)(F)F)(C3)C4)c2nc1C] |
| 92 | 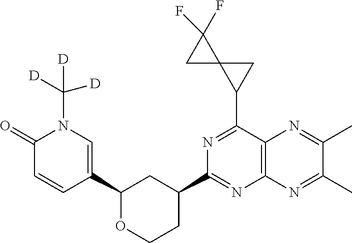 [SMILES: [2H]C([2H])([2H])n1cc([C@H]2C[C@@H](c3nc(C4CC45CC5(F)F)c4nc(C)c(C)nc4n3)CCO2)ccc1=O] |
| 93 | 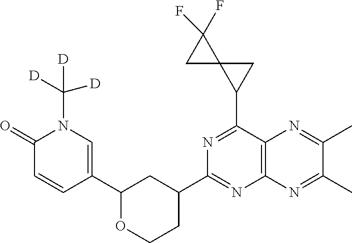 [SMILES: [2H]C([2H])([2H])n1cc(C2CC(c3nc(C4CC45CC5(F)F)c4nc(C)c(C)nc4n3)CCO2)ccc1=O] |
| 94 | 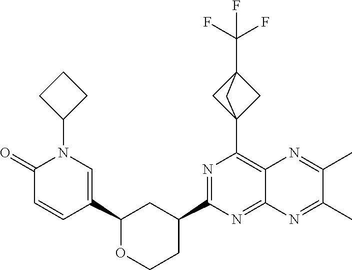 [SMILES: Cc1nc2nc([C@H]3CCO[C@@H](c4ccc(=O)n(C5CCC5)c4)C3)nc(C34CC(C(F)(F)F)(C3)C4)c2nc1C] |
| 95 | 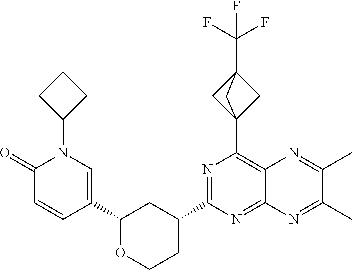 [SMILES: Cc1nc2nc([C@@H]3CCO[C@H](c4ccc(=O)n(C5CCC5)c4)C3)nc(C34CC(C(F)(F)F)(C3)C4)c2nc1C] |
| 96 | 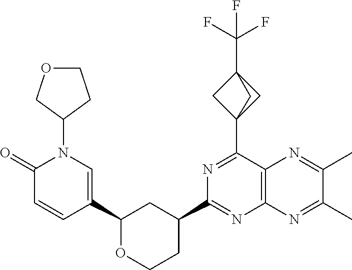 [SMILES: Cc1nc2nc([C@H]3CCO[C@@H](c4ccc(=O)n(C5CCOC5)c4)C3)nc(C34CC(C(F)(F)F)(C3)C4)c2nc1C] |
| 97 | 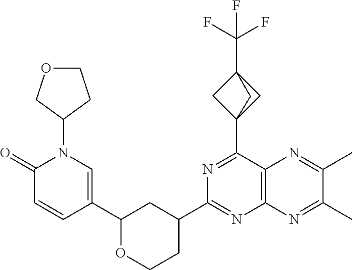 [SMILES: Cc1nc2nc(C3CCOC(c4ccc(=O)n(C5CCOC5)c4)C3)nc(C34CC(C(F)(F)F)(C3)C4)c2nc1C] |
| 98 | 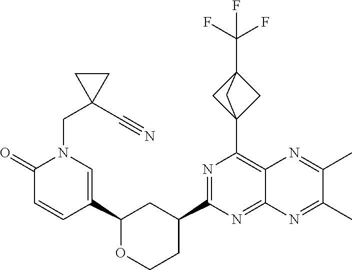 [SMILES: Cc1nc2nc([C@H]3CCO[C@@H](c4ccc(=O)n(CC5(C#N)CC5)c4)C3)nc(C34CC(C(F)(F)F)(C3)C4)c2nc1C] |
| 99 | 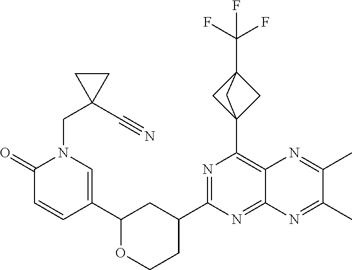 [SMILES: Cc1nc2nc(C3CCOC(c4ccc(=O)n(CC5(C#N)CC5)c4)C3)nc(C34CC(C(F)(F)F)(C3)C4)c2nc1C] |
| 100 | 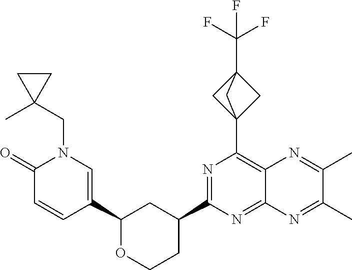 [SMILES: Cc1nc2nc([C@H]3CCO[C@@H](c4ccc(=O)n(CC5(C)CC5)c4)C3)nc(C34CC(C(F)(F)F)(C3)C4)c2nc1C] |
| 101 | 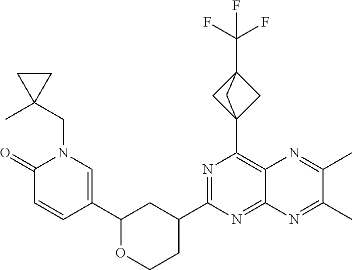 [SMILES: Cc1nc2nc(C3CCOC(c4ccc(=O)n(CC5(C)CC5)c4)C3)nc(C34CC(C(F)(F)F)(C3)C4)c2nc1C] |
| 102 | 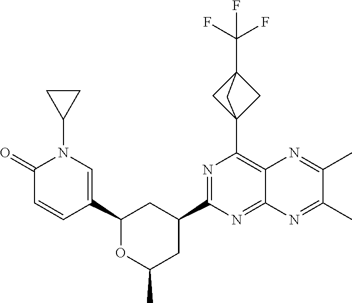 [SMILES: Cc1nc2nc([C@H]3C[C@@H](C)O[C@@H](c4ccc(=O)n(C5CC5)c4)C3)nc(C34CC(C(F)(F)F)(C3)C4)c2nc1C] |
| 103 | 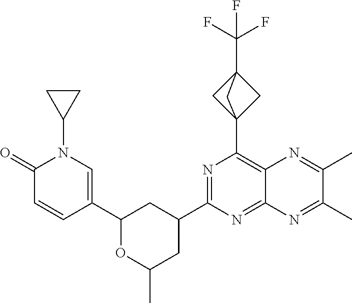 [SMILES: Cc1nc2nc(C3CC(C)OC(c4ccc(=O)n(C5CC5)c4)C3)nc(C34CC(C(F)(F)F)(C3)C4)c2nc1C] |
| 104 | 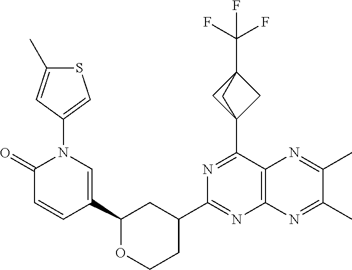 [SMILES: Cc1cc(-n2cc([C@H]3CC(c4nc(C56CC(C(F)(F)F)(C5)C6)c5nc(C)c(C)nc5n4)CCO3)ccc2=O)cs1] |
| 105 | 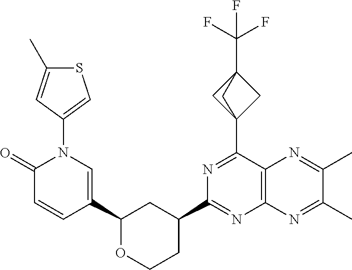 [SMILES: Cc1cc(-n2cc([C@H]3C[C@@H](c4nc(C56CC(C(F)(F)F)(C5)C6)c5nc(C)c(C)nc5n4)CCO3)ccc2=O)cs1] |
| 106 | 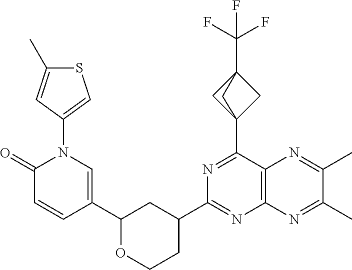 [SMILES: Cc1cc(-n2cc(C3CC(c4nc(C56CC(C(F)(F)F)(C5)C6)c5nc(C)c(C)nc5n4)CCO3)ccc2=O)cs1] |
| 107 | 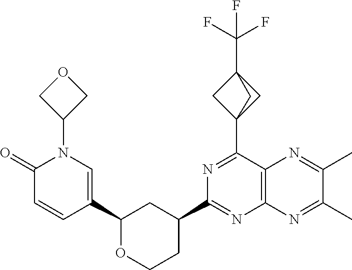 [SMILES: Cc1nc2nc([C@H]3CCO[C@@H](c4ccc(=O)n(C5COC5)c4)C3)nc(C34CC(C(F)(F)F)(C3)C4)c2nc1C] |
| 108 | 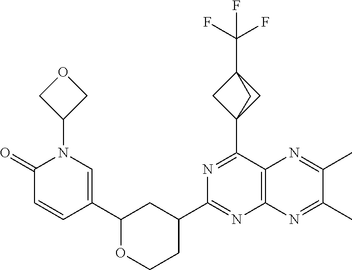 [SMILES: Cc1nc2nc(C3CCOC(c4ccc(=O)n(C5COC5)c4)C3)nc(C34CC(C(F)(F)F)(C3)C4)c2nc1C] |
| 109 | 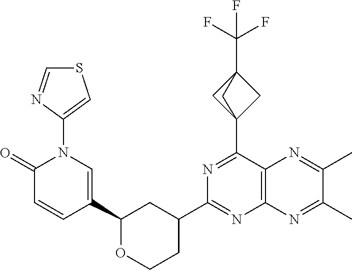 [SMILES: Cc1nc2nc(C3CCO[C@@H](c4ccc(=O)n(-c5cscn5)c4)C3)nc(C34CC(C(F)(F)F)(C3)C4)c2nc1C] |
| 110 | 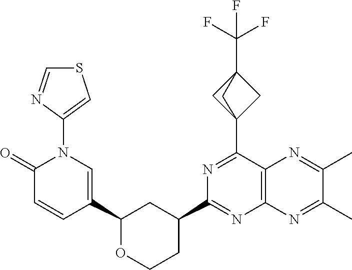 [SMILES: Cc1nc2nc([C@H]3CCO[C@@H](c4ccc(=O)n(-c5cscn5)c4)C3)nc(C34CC(C(F)(F)F)(C3)C4)c2nc1C] |
| 111 | 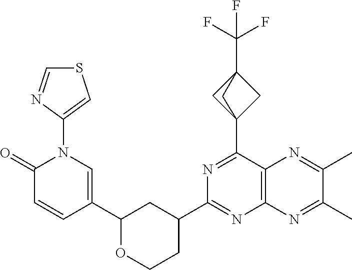 [SMILES: Cc1nc2nc(C3CCOC(c4ccc(=O)n(-c5cscn5)c4)C3)nc(C34CC(C(F)(F)F)(C3)C4)c2nc1C] |
| 112 | 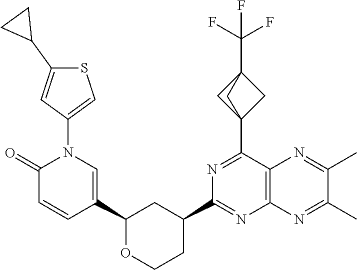 [SMILES: Cc1nc2nc([C@H]3CCO[C@@H](c4ccc(=O)n(-c5csc(C6CC6)c5)c4)C3)nc(C34CC(C(F)(F)F)(C3)C4)c2nc1C] |
| 113 | 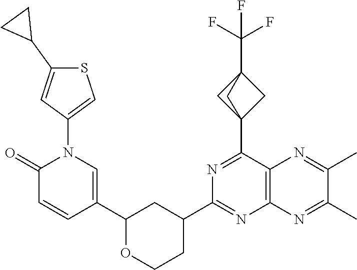 [SMILES: Cc1nc2nc(C3CCOC(c4ccc(=O)n(-c5csc(C6CC6)c5)c4)C3)nc(C34CC(C(F)(F)F)(C3)C4)c2nc1C] |
| 114 | 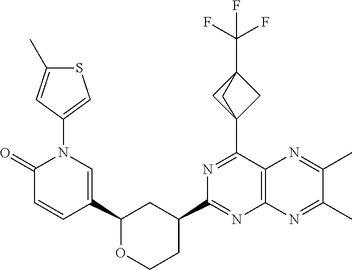 [SMILES: Cc1cc(-n2cc([C@H]3C[C@@H](c4nc(C56CC(C(F)(F)F)(C5)C6)c5nc(C)c(C)nc5n4)CCO3)ccc2=O)cs1] |
| 115 | 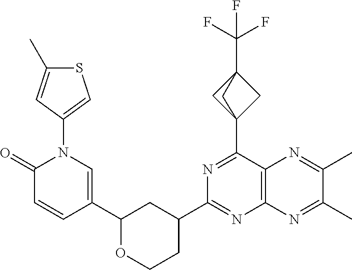 [SMILES: Cc1cc(-n2cc(C3CC(c4nc(C56CC(C(F)(F)F)(C5)C6)c5nc(C)c(C)nc5n4)CCO3)ccc2=O)cs1] |
| 116 | 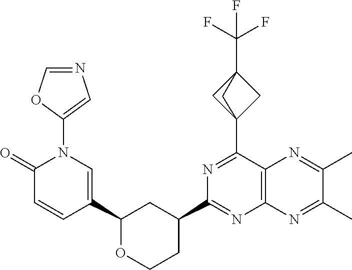 [SMILES: Cc1nc2nc([C@H]3CCO[C@@H](c4ccc(=O)n(-c5cnco5)c4)C3)nc(C34CC(C(F)(F)F)(C3)C4)c2nc1C] |
| 117 | 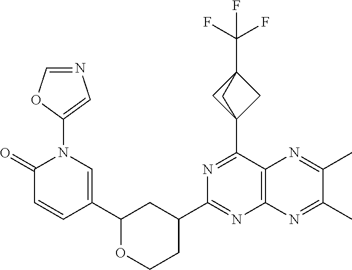 [SMILES: Cc1nc2nc(C3CCOC(c4ccc(=O)n(-c5cnco5)c4)C3)nc(C34CC(C(F)(F)F)(C3)C4)c2nc1C] |
| 118 | 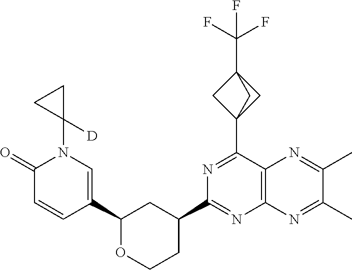 [SMILES: [2H]C1(n2cc([C@H]3C[C@@H](c4nc(C56CC(C(F)(F)F)(C5)C6)c5nc(C)c(C)nc5n4)CCO3)ccc2=O)CC1] |
| 119 | 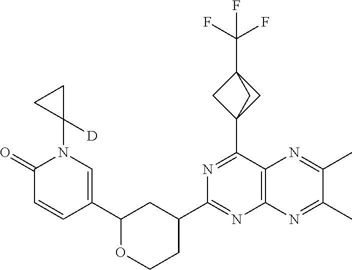 [SMILES: [2H]C1(n2cc(C3CC(c4nc(C56CC(C(F)(F)F)(C5)C6)c5nc(C)c(C)nc5n4)CCO3)ccc2=O)CC1] |
| 120 | 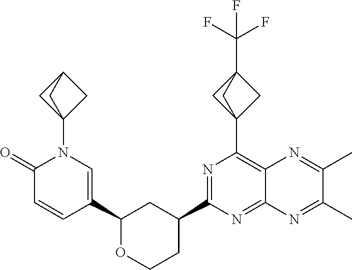 [SMILES: Cc1nc2nc([C@H]3CCO[C@@H](c4ccc(=O)n(C56CC(C5)C6)c4)C3)nc(C34CC(C(F)(F)F)(C3)C4)c2nc1C] |
| 121 | 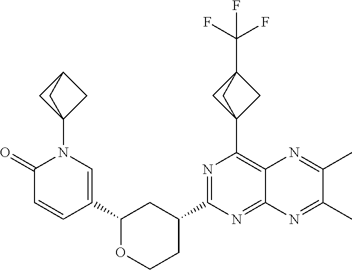 [SMILES: Cc1nc2nc([C@@H]3CCO[C@H](c4ccc(=O)n(C56CC(C5)C6)c4)C3)nc(C34CC(C(F)(F)F)(C3)C4)c2nc1C] |
| 122 | 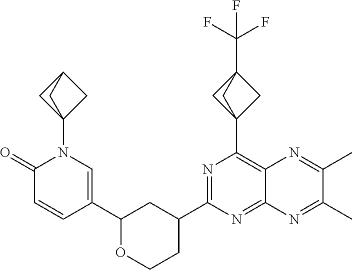 [SMILES: Cc1nc2nc(C3CCOC(c4ccc(=O)n(C56CC(C5)C6)c4)C3)nc(C34CC(C(F)(F)F)(C3)C4)c2nc1C] |
| 123 | 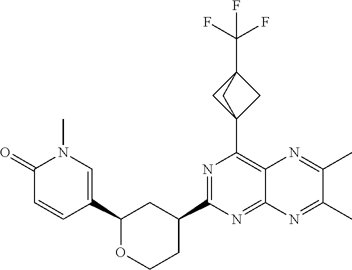 [SMILES: Cc1nc2nc([C@H]3CCO[C@@H](c4ccc(=O)n(C)c4)C3)nc(C34CC(C(F)(F)F)(C3)C4)c2nc1C] |
| 124 | 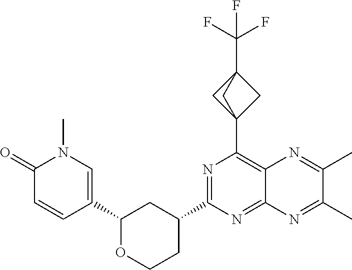 [SMILES: Cc1nc2nc([C@@H]3CCO[C@H](c4ccc(=O)n(C)c4)C3)nc(C34CC(C(F)(F)F)(C3)C4)c2nc1C] |
| 125 | 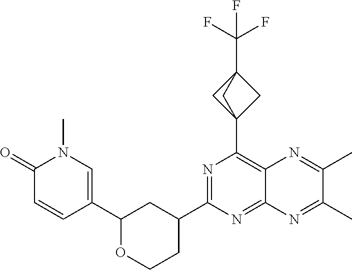 [SMILES: Cc1nc2nc(C3CCOC(c4ccc(=O)n(C)c4)C3)nc(C34CC(C(F)(F)F)(C3)C4)c2nc1C] |
| 126 | 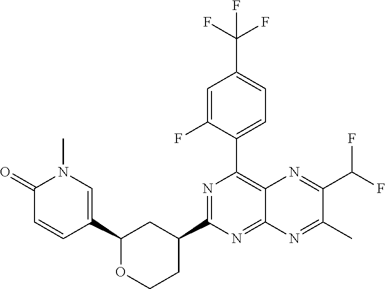 [SMILES: Cc1nc2nc([C@H]3CCO[C@@H](c4ccc(=O)n(C)c4)C3)nc(-c3ccc(C(F)(F)F)cc3F)c2nc1C(F)F] |
| 127 | 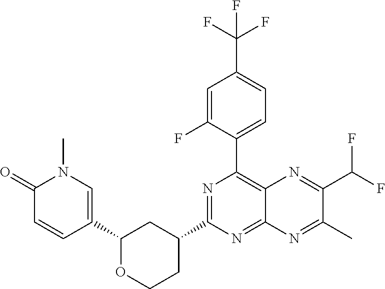 [SMILES: Cc1nc2nc([C@@H]3CCO[C@H](c4ccc(=O)n(C)c4)C3)nc(-c3ccc(C(F)(F)F)cc3F)c2nc1C(F)F] |
| 128 | 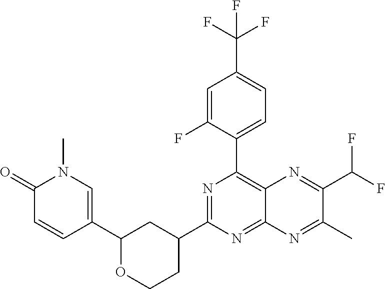 [SMILES: Cc1nc2nc(C3CCOC(c4ccc(=O)n(C)c4)C3)nc(-c3ccc(C(F)(F)F)cc3F)c2nc1C(F)F] |
| 129 | 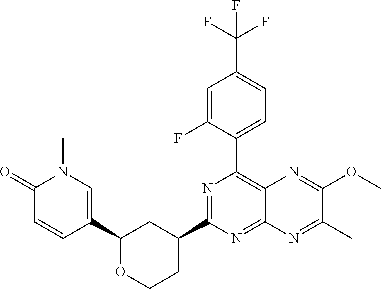 [SMILES: COc1nc2c(-c3ccc(C(F)(F)F)cc3F)nc([C@H]3CCO[C@@H](c4ccc(=O)n(C)c4)C3)nc2nc1C] |
| 130 | 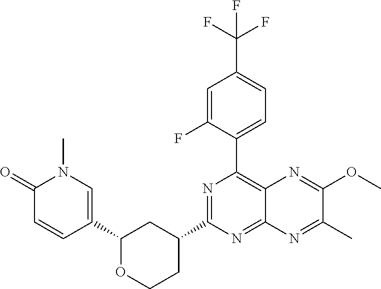 [SMILES: COc1nc2c(-c3ccc(C(F)(F)F)cc3F)nc([C@@H]3CCO[C@H](c4ccc(=O)n(C)c4)C3)nc2nc1C] |
| 131 | 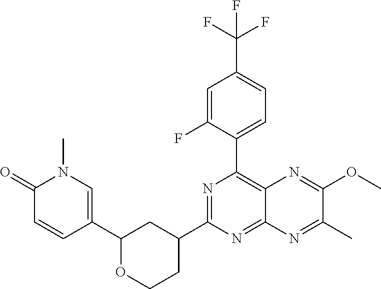 [SMILES: COc1nc2c(-c3ccc(C(F)(F)F)cc3F)nc(C3CCOC(c4ccc(=O)n(C)c4)C3)nc2nc1C] |
| 132 | 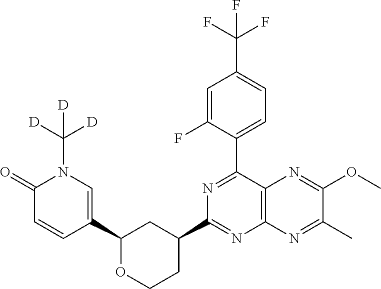 [SMILES: [2H]C([2H])([2H])n1cc([C@H]2C[C@@H](c3nc(-c4ccc(C(F)(F)F)cc4F)c4nc(OC)c(C)nc4n3)CCO2)ccc1=O] |
| 133 | 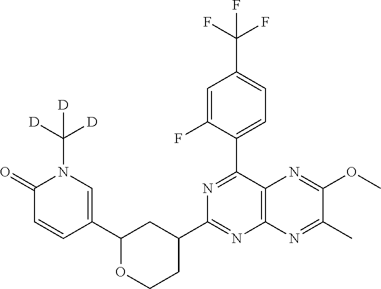 [SMILES: [2H]C([2H])([2H])n1cc(C2CC(c3nc(-c4ccc(C(F)(F)F)cc4F)c4nc(OC)c(C)nc4n3)CCO2)ccc1=O] |
| 134 | 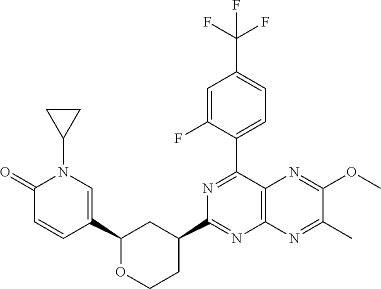 [SMILES: COc1nc2c(-c3ccc(C(F)(F)F)cc3F)nc([C@H]3CCO[C@@H](c4ccc(=O)n(C5CC5)c4)C3)nc2nc1C] |
| 135 | 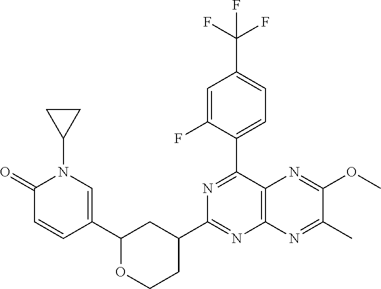 [SMILES: COc1nc2c(-c3ccc(C(F)(F)F)cc3F)nc(C3CCOC(c4ccc(=O)n(C5CC5)c4)C3)nc2nc1C] |
| 136 | 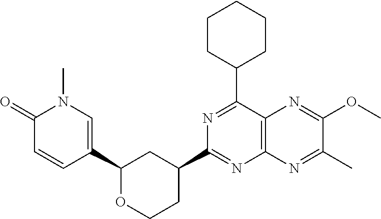 [SMILES: COc1nc2c(C3CCCCC3)nc([C@H]3CCO[C@@H](c4ccc(=O)n(C)c4)C3)nc2nc1C] |
| 137 | 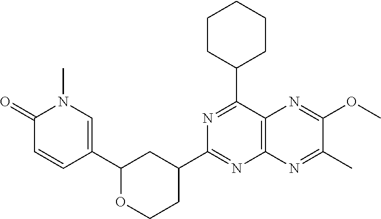 [SMILES: COc1nc2c(C3CCCCC3)nc(C3CCOC(c4ccc(=O)n(C)c4)C3)nc2nc1C] |
| 138 | 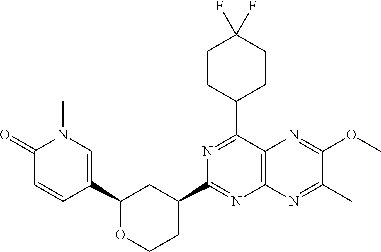 [SMILES: COc1nc2c(C3CCC(F)(F)CC3)nc([C@H]3CCO[C@@H](c4ccc(=O)n(C)c4)C3)nc2nc1C] |
| 139 | 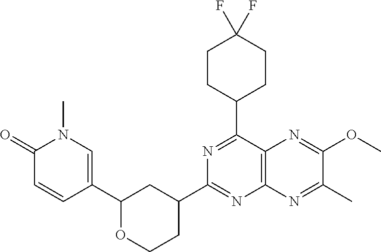 [SMILES: COc1nc2c(C3CCC(F)(F)CC3)nc(C3CCOC(c4ccc(=O)n(C)c4)C3)nc2nc1C] |
| 140 | 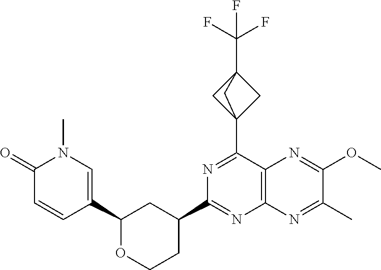 [SMILES: COc1nc2c(C34CC(C(F)(F)F)(C3)C4)nc([C@H]3CCO[C@@H](c4ccc(=O)n(C)c4)C3)nc2nc1C] |
| 141 | 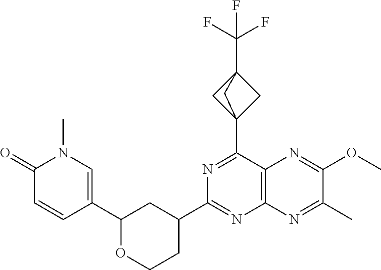 [SMILES: COc1nc2c(C34CC(C(F)(F)F)(C3)C4)nc(C3CCOC(c4ccc(=O)n(C)c4)C3)nc2nc1C] |
| 142 | 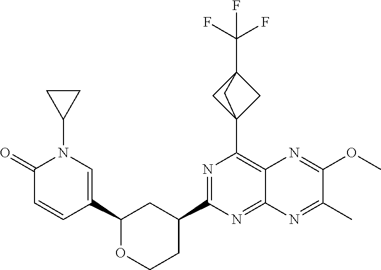 [SMILES: COc1nc2c(C34CC(C(F)(F)F)(C3)C4)nc([C@H]3CCO[C@@H](c4ccc(=O)n(C5CC5)c4)C3)nc2nc1C] |
| 143 | 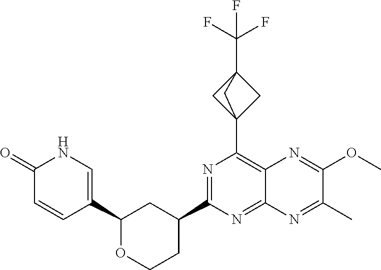 [SMILES: COc1nc2c(C34CC(C(F)(F)F)(C3)C4)nc([C@H]3CCO[C@@H](c4ccc(=O)[nH]c4)C3)nc2nc1C] |
| 144 | 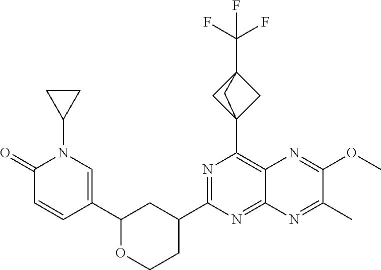 [SMILES: COc1nc2c(C34CC(C(F)(F)F)(C3)C4)nc(C3CCOC(c4ccc(=O)n(C5CC5)c4)C3)nc2nc1C] |
| 145 | 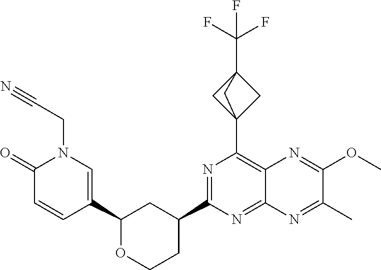 [SMILES: COc1nc2c(C34CC(C(F)(F)F)(C3)C4)nc([C@H]3CCO[C@@H](c4ccc(=O)n(CC#N)c4)C3)nc2nc1C] |
| 146 | 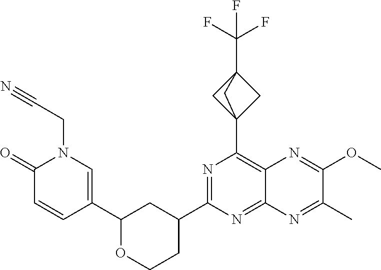 [SMILES: COc1nc2c(C34CC(C(F)(F)F)(C3)C4)nc(C3CCOC(c4ccc(=O)n(CC#N)c4)C3)nc2nc1C] |
| 147 | 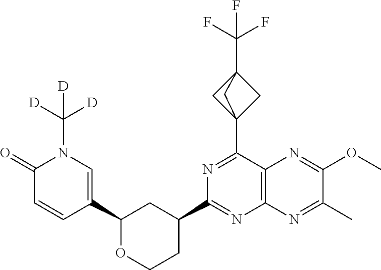 [SMILES: [2H]C([2H])([2H])n1cc([C@H]2C[C@@H](c3nc(C45CC(C(F)(F)F)(C4)C5)c4nc(OC)c(C)nc4n3)CCO2)ccc1=O] |
| 148 | 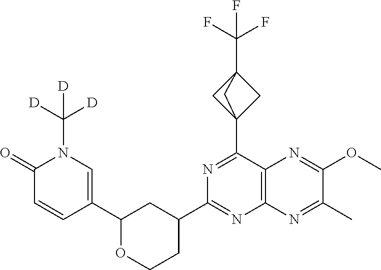 [SMILES: [2H]C([2H])([2H])n1cc(C2CC(c3nc(C45CC(C(F)(F)F)(C4)C5)c4nc(OC)c(C)nc4n3)CCO2)ccc1=O] |
| 149 | 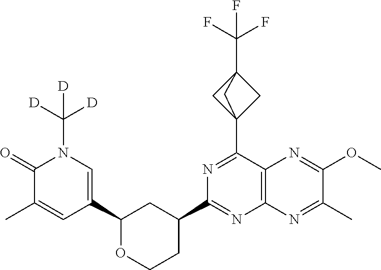 [SMILES: [2H]C([2H])([2H])n1cc([C@H]2C[C@@H](c3nc(C45CC(C(F)(F)F)(C4)C5)c4nc(OC)c(C)nc4n3)CCO2)cc(C)c1=O] |
| 150 | 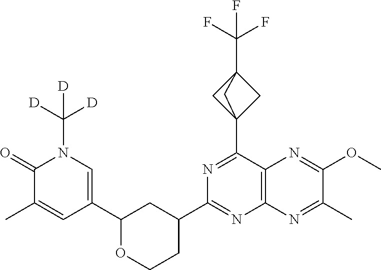 [SMILES: [2H]C([2H])([2H])n1cc(C2CC(c3nc(C45CC(C(F)(F)F)(C4)C5)c4nc(OC)c(C)nc4n3)CCO2)cc(C)c1=O] |
| 151 | 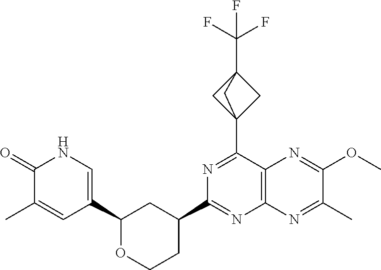 [SMILES: COc1nc2c(C34CC(C(F)(F)F)(C3)C4)nc([C@H]3CCO[C@@H](c4c[nH]c(=O)c(C)c4)C3)nc2nc1C] |
| 152 | 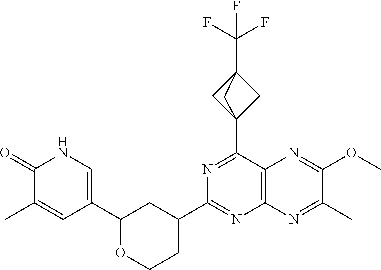 |
| 153 | 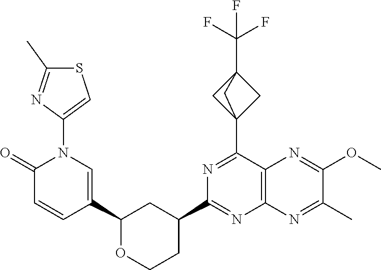 [SMILES: COc1nc2c(C34CC(C(F)(F)F)(C3)C4)nc([C@H]3CCO[C@@H](c4ccc(=O)n(-c5csc(C)n5)c4)C3)nc2nc1C] |
| 154 | 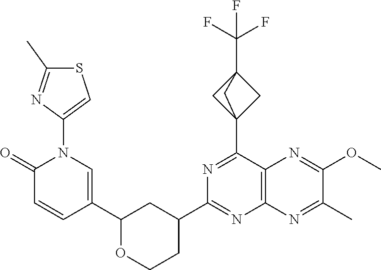 [SMILES: COc1nc2c(C34CC(C(F)(F)F)(C3)C4)nc(C3CCOC(c4ccc(=O)n(-c5csc(C)n5)c4)C3)nc2nc1C] |
| 155 | 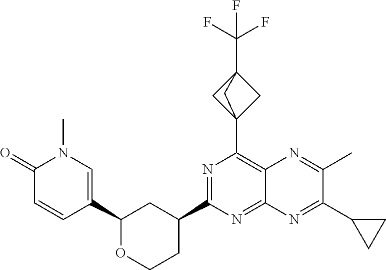 [SMILES: Cc1nc2c(C34CC(C(F)(F)F)(C3)C4)nc([C@H]3CCO[C@@H](c4ccc(=O)n(C)c4)C3)nc2nc1C1CC1] |
| 156 | 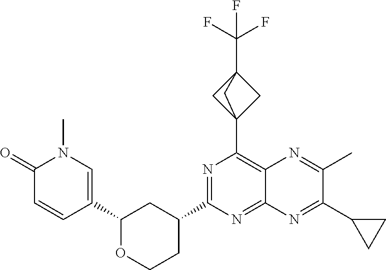 [SMILES: Cc1nc2c(C34CC(C(F)(F)F)(C3)C4)nc([C@@H]3CCO[C@H](c4ccc(=O)n(C)c4)C3)nc2nc1C1CC1] |
| 157 | 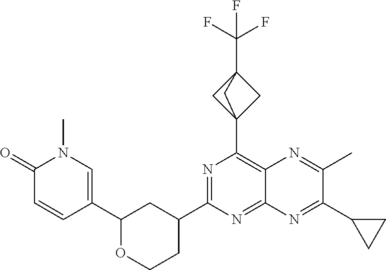 [SMILES: Cc1nc2c(C34CC(C(F)(F)F)(C3)C4)nc(C3CCOC(c4ccc(=O)n(C)c4)C3)nc2nc1C1CC1] |
| 158 | 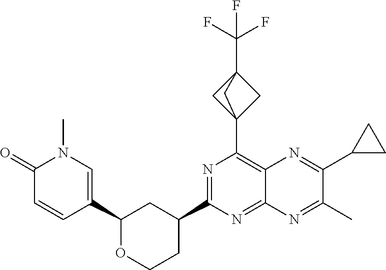 [SMILES: Cc1nc2nc([C@H]3CCO[C@@H](c4ccc(=O)n(C)c4)C3)nc(C34CC(C(F)(F)F)(C3)C4)c2nc1C1CC1] |
| 159 | 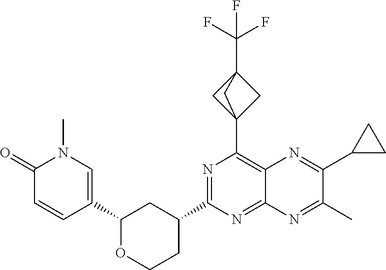 [SMILES: Cc1nc2nc([C@@H]3CCO[C@H](c4ccc(=O)n(C)c4)C3)nc(C34CC(C(F)(F)F)(C3)C4)c2nc1C1CC1] |
| 160 | 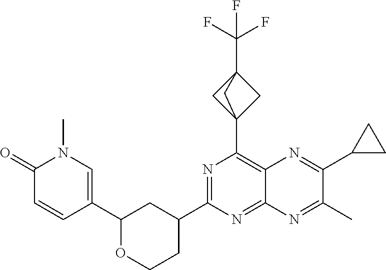 [SMILES: Cc1nc2nc(C3CCOC(c4ccc(=O)n(C)c4)C3)nc(C34CC(C(F)(F)F)(C3)C4)c2nc1C1CC1] |
| 161 | 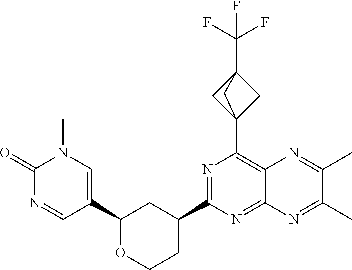 [SMILES: Cc1nc2nc([C@H]3CCO[C@@H](c4cnc(=O)n(C)c4)C3)nc(C34CC(C(F)(F)F)(C3)C4)c2nc1C] |
| 162 | 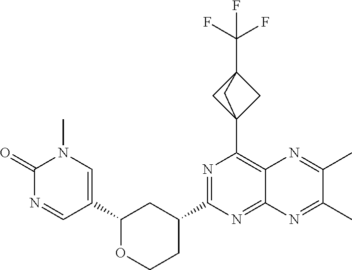 [SMILES: Cc1nc2nc([C@@H]3CCO[C@H](c4cnc(=O)n(C)c4)C3)nc(C34CC(C(F)(F)F)(C3)C4)c2nc1C] |
| 163 | 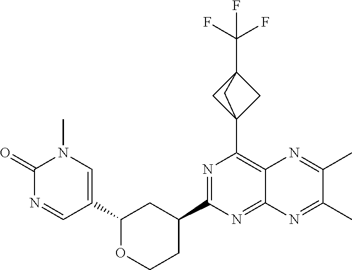 [SMILES: Cc1nc2nc([C@H]3CCO[C@H](c4cnc(=O)n(C)c4)C3)nc(C34CC(C(F)(F)F)(C3)C4)c2nc1C] |
| 164 | 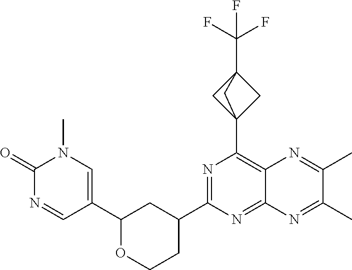 [SMILES: Cc1nc2nc(C3CCOC(c4cnc(=O)n(C)c4)C3)nc(C34CC(C(F)(F)F)(C3)C4)c2nc1C] |
| 165 | 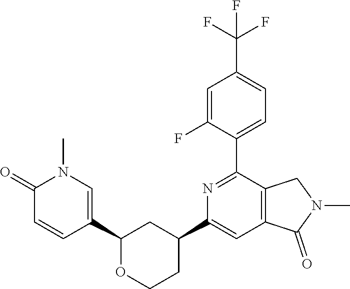 [SMILES: CN1Cc2c(cc([C@H]3CCO[C@@H](c4ccc(=O)n(C)c4)C3)nc2-c2ccc(C(F)(F)F)cc2F)C1=O] |
| 166 | 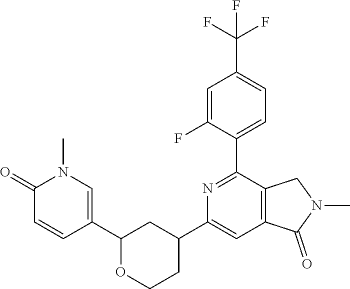 [SMILES: CN1Cc2c(cc(C3CCOC(c4ccc(=O)n(C)c4)C3)nc2-c2ccc(C(F)(F)F)cc2F)C1=O] |
| 167 | 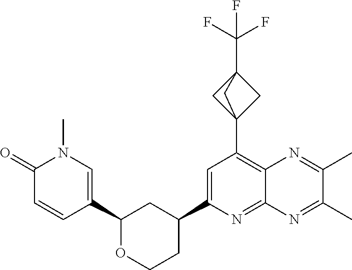 [SMILES: Cc1nc2nc([C@H]3CCO[C@@H](c4ccc(=O)n(C)c4)C3)cc(C34CC(C(F)(F)F)(C3)C4)c2nc1C] |
| 168 | 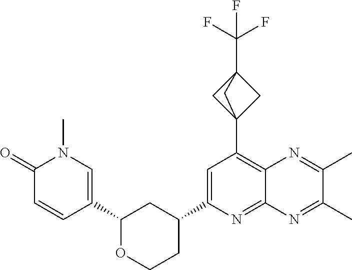 [SMILES: Cc1nc2nc([C@@H]3CCO[C@H](c4ccc(=O)n(C)c4)C3)cc(C34CC(C(F)(F)F)(C3)C4)c2nc1C] |
| 169 | 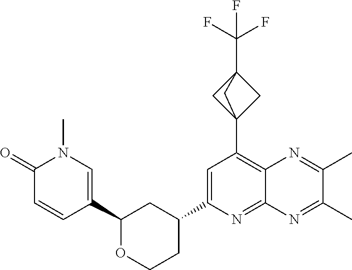 [SMILES: Cc1nc2nc([C@@H]3CCO[C@@H](c4ccc(=O)n(C)c4)C3)cc(C34CC(C(F)(F)F)(C3)C4)c2nc1C] |
| 170 | 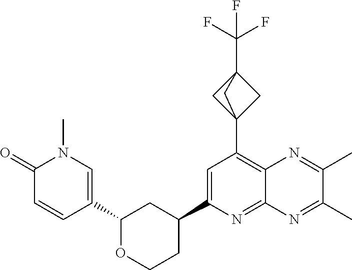 [SMILES: Cc1nc2nc([C@H]3CCO[C@H](c4ccc(=O)n(C)c4)C3)cc(C34CC(C(F)(F)F)(C3)C4)c2nc1C] |
| 171 | 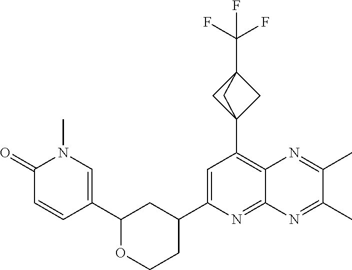 [SMILES: Cc1nc2nc(C3CCOC(c4ccc(=O)n(C)c4)C3)cc(C34CC(C(F)(F)F)(C3)C4)c2nc1C] |
| 177 | 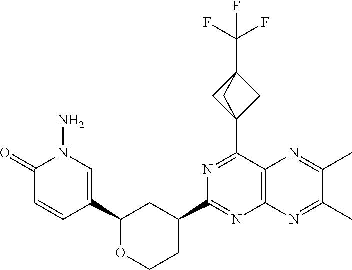 [SMILES: Cc1nc2nc([C@H]3CCO[C@@H](c4ccc(=O)n(N)c4)C3)nc(C34CC(C(F)(F)F)(C3)C4)c2nc1C] |
| 178 | 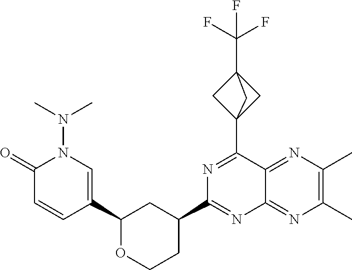 [SMILES: Cc1nc2nc([C@H]3CCO[C@@H](c4ccc(=O)n(N(C)C)c4)C3)nc(C34CC(C(F)(F)F)(C3)C4)c2nc1C] |
| 179 | 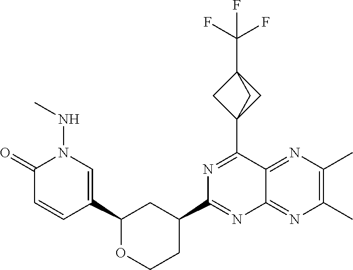 [SMILES: CNn1cc([C@H]2C[C@@H](c3nc(C45CC(C(F)(F)F)(C4)C5)c4nc(C)c(C)nc4n3)CCO2)ccc1=O] |
| 181 | 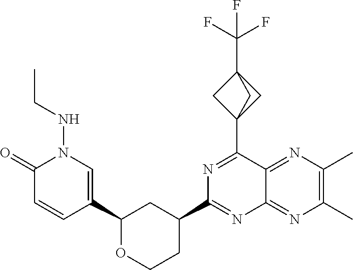 [SMILES: CCNn1cc([C@H]2C[C@@H](c3nc(C45CC(C(F)(F)F)(C4)C5)c4nc(C)c(C)nc4n3)CCO2)ccc1=O] |
| 187 | 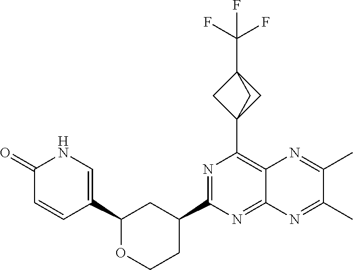 [SMILES: Cc1nc2nc([C@H]3CCO[C@@H](c4ccc(=O)[nH]c4)C3)nc(C34CC(C(F)(F)F)(C3)C4)c2nc1C] |
| 188 | 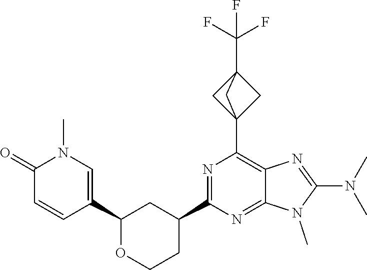 [SMILES: CN(C)c1nc2c(C34CC(C(F)(F)F)(C3)C4)nc([C@H]3CCO[C@@H](c4ccc(=O)n(C)c4)C3)nc2n1C] |
| 194 | 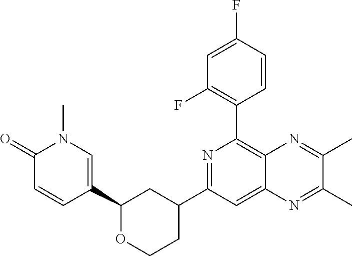 [SMILES: Cc1nc2cc(C3CCO[C@@H](c4ccc(=O)n(C)c4)C3)nc(-c3ccc(F)cc3F)c2nc1C] |
| 197 | 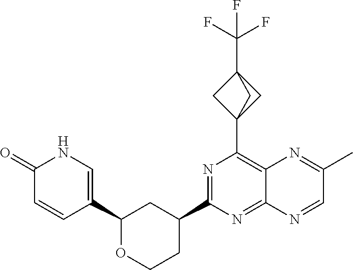 [SMILES: Cc1cnc2nc([C@H]3CCO[C@@H](c4ccc(=O)[nH]c4)C3)nc(C34CC(C(F)(F)F)(C3)C4)c2n1] |
| 198 | 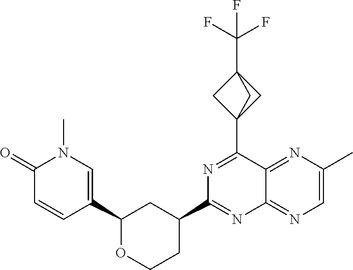 [SMILES: Cc1cnc2nc([C@H]3CCO[C@@H](c4ccc(=O)n(C)c4)C3)nc(C34CC(C(F)(F)F)(C3)C4)c2n1] |
| 201 | 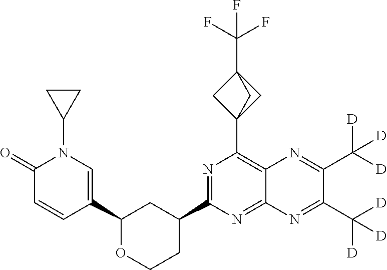 [SMILES: [2H]C([2H])([2H])c1nc2nc([C@H]3CCO[C@@H](c4ccc(=O)n(C5CC5)c4)C3)nc(C34CC(C(F)(F)F)(C3)C4)c2nc1C([2H])([2H])[2H]] |
| 202 | 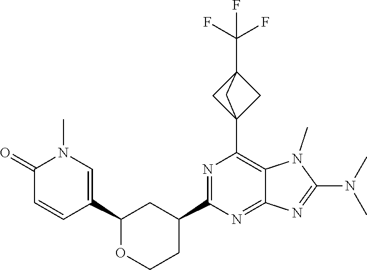 [SMILES: CN(C)c1nc2nc([C@H]3CCO[C@@H](c4ccc(=O)n(C)c4)C3)nc(C34CC(C(F)(F)F)(C3)C4)c2n1C] |
| 203 | 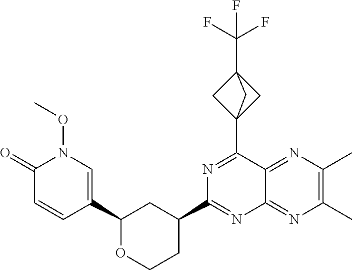 [SMILES: COn1cc([C@H]2C[C@@H](c3nc(C45CC(C(F)(F)F)(C4)C5)c4nc(C)c(C)nc4n3)CCO2)ccc1=O] |
| 206 | 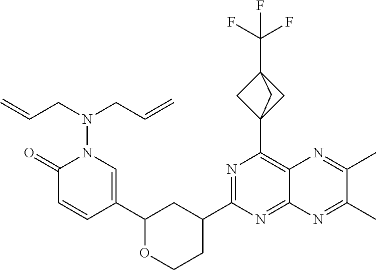 [SMILES: C=CCN(CC=C)n1cc(C2CC(c3nc(C45CC(C(F)(F)F)(C4)C5)c4nc(C)c(C)nc4n3)CCO2)ccc1=O] |
| 207 | 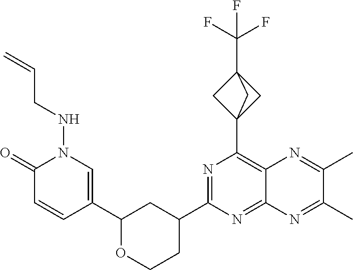 [SMILES: C=CCNn1cc(C2CC(c3nc(C45CC(C(F)(F)F)(C4)C5)c4nc(C)c(C)nc4n3)CCO2)ccc1=O] |
| 208 | 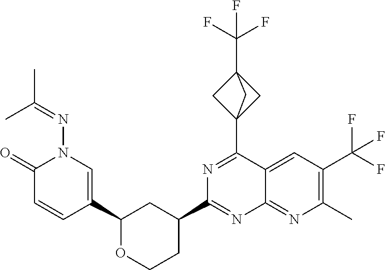 [SMILES: CC(C)=Nn1cc([C@H]2C[C@@H](c3nc(C45CC(C(F)(F)F)(C4)C5)c4cc(C(F)(F)F)c(C)nc4n3)CCO2)ccc1=O] |
| 209 | 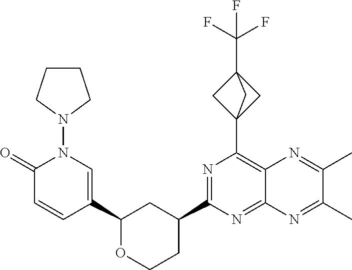 [SMILES: Cc1nc2nc([C@H]3CCO[C@@H](c4ccc(=O)n(N5CCCC5)c4)C3)nc(C34CC(C(F)(F)F)(C3)C4)c2nc1C] |
| 210 | 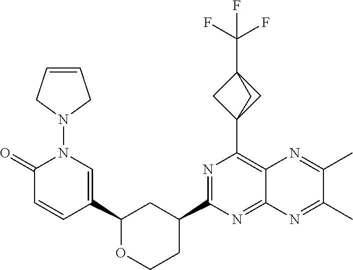 [SMILES: Cc1nc2nc([C@H]3CCO[C@@H](c4ccc(=O)n(N5CC=CC5)c4)C3)nc(C34CC(C(F)(F)F)(C3)C4)c2nc1C] |
| 211 | 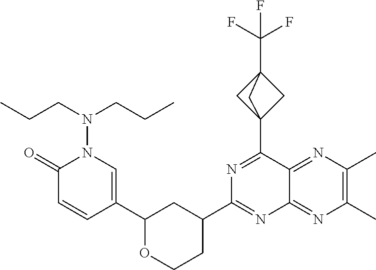 [SMILES: CCCN(CCC)n1cc(C2CC(c3nc(C45CC(C(F)(F)F)(C4)C5)c4nc(C)c(C)nc4n3)CCO2)ccc1=O] |
| 212 | 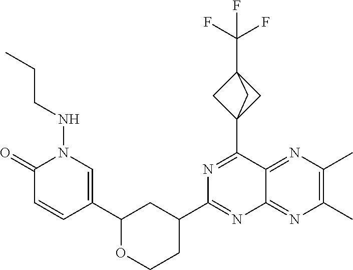 [SMILES: CCCNn1cc(C2CC(c3nc(C45CC(C(F)(F)F)(C4)C5)c4nc(C)c(C)nc4n3)CCO2)ccc1=O] |
| 213 | 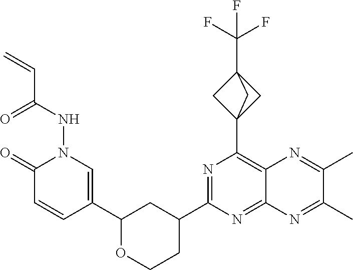 [SMILES: C=CC(=O)Nn1cc(C2CC(c3nc(C45CC(C(F)(F)F)(C4)C5)c4nc(C)c(C)nc4n3)CCO2)ccc1=O] |
| 214 | 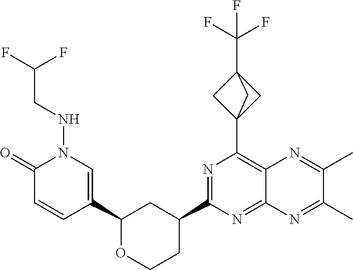 [SMILES: Cc1nc2nc([C@H]3CCO[C@@H](c4ccc(=O)n(NCC(F)F)c4)C3)nc(C34CC(C(F)(F)F)(C3)C4)c2nc1C] |
| 216 | 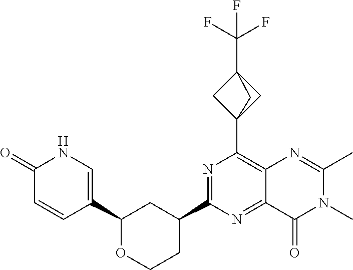 [SMILES: Cc1nc2c(C34CC(C(F)(F)F)(C3)C4)nc([C@H]3CCO[C@@H](c4ccc(=O)[nH]c4)C3)nc2c(=O)n1C] |
| 217 | 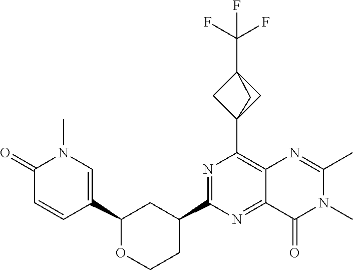 [SMILES: Cc1nc2c(C34CC(C(F)(F)F)(C3)C4)nc([C@H]3CCO[C@@H](c4ccc(=O)n(C)c4)C3)nc2c(=O)n1C] |
| 218 | 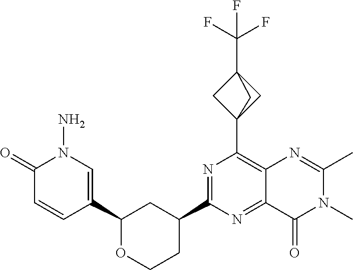 [SMILES: Cc1nc2c(C34CC(C(F)(F)F)(C3)C4)nc([C@H]3CCO[C@@H](c4ccc(=O)n(N)c4)C3)nc2c(=O)n1C] |
| 220 | 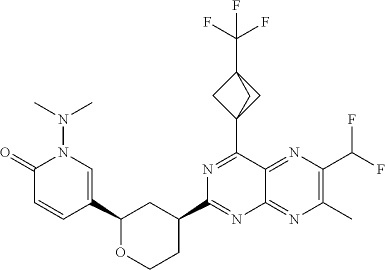 [SMILES: Cc1nc2nc([C@H]3CCO[C@@H](c4ccc(=O)n(N(C)C)c4)C3)nc(C34CC(C(F)(F)F)(C3)C4)c2nc1C(F)F] |
| 224 | 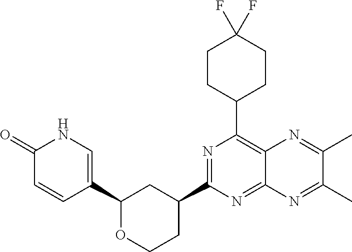 [SMILES: Cc1nc2nc([C@H]3CCO[C@@H](c4ccc(=O)[nH]c4)C3)nc(C3CCC(F)(F)CC3)c2nc1C] |
| 225 | 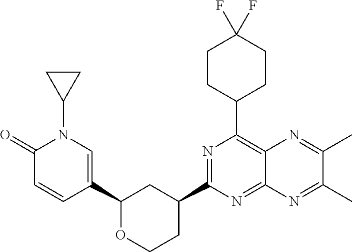 [SMILES: Cc1nc2nc([C@H]3CCO[C@@H](c4ccc(=O)n(C5CC5)c4)C3)nc(C3CCC(F)(F)CC3)c2nc1C] |
| 226 | 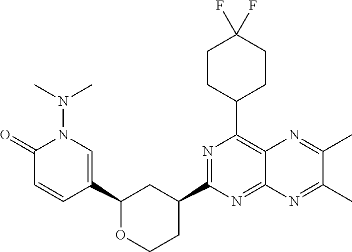 [SMILES: Cc1nc2nc([C@H]3CCO[C@@H](c4ccc(=O)n(N(C)C)c4)C3)nc(C3CCC(F)(F)CC3)c2nc1C] |
| 227 | 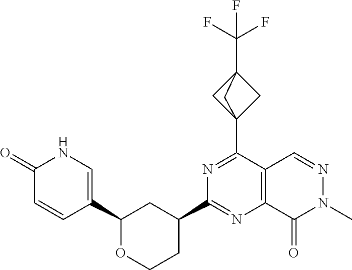 [SMILES: Cn1ncc2c(C34CC(C(F)(F)F)(C3)C4)nc([C@H]3CCO[C@@H](c4ccc(=O)[nH]c4)C3)nc2c1=O] |
| 228 | 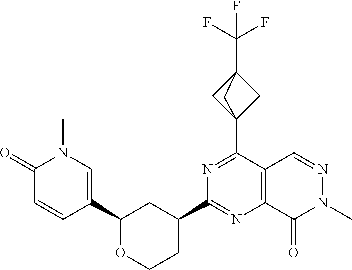 [SMILES: Cn1cc([C@H]2C[C@@H](c3nc(C45CC(C(F)(F)F)(C4)C5)c4cnn(C)c(=O)c4n3)CCO2)ccc1=O] |
| 229 | 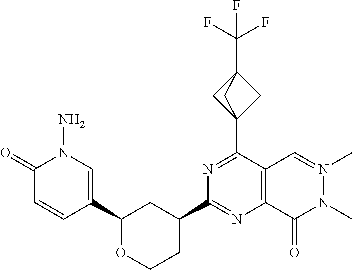 |
| 230 | 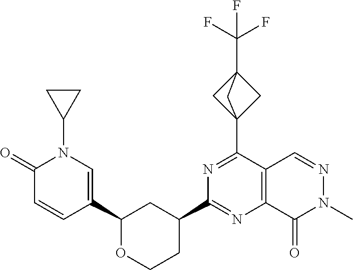 [SMILES: Cn1ncc2c(C34CC(C(F)(F)F)(C3)C4)nc([C@H]3CCO[C@@H](c4ccc(=O)n(C5CC5)c4)C3)nc2c1=O] |
| 231 | 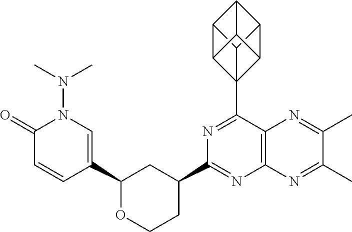 [SMILES: Cc1nc2nc([C@H]3CCO[C@@H](c4ccc(=O)n(N(C)C)c4)C3)nc(C34C5C6C7C5C3C7C64)c2nc1C] |
| 232 | 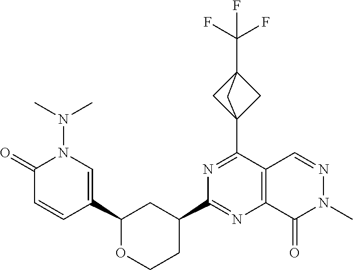 [SMILES: CN(C)n1cc([C@H]2C[C@@H](c3nc(C45CC(C(F)(F)F)(C4)C5)c4cnn(C)c(=O)c4n3)CCO2)ccc1=O] |
| 233 | 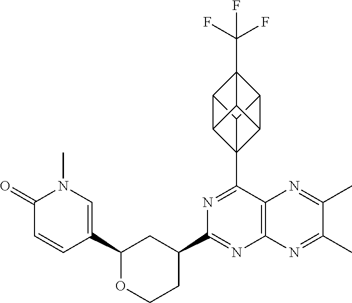 [SMILES: Cc1nc2nc([C@H]3CCO[C@@H](c4ccc(=O)n(C)c4)C3)nc(C34C5C6C3C3C4C5C63C(F)(F)F)c2nc1C] |
| 234 | 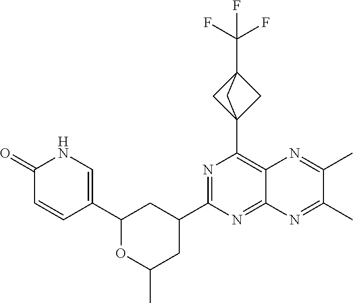 [SMILES: Cc1nc2nc(C3CC(C)OC(c4ccc(=O)[nH]c4)C3)nc(C34CC(C(F)(F)F)(C3)C4)c2nc1C] |
| 235 | 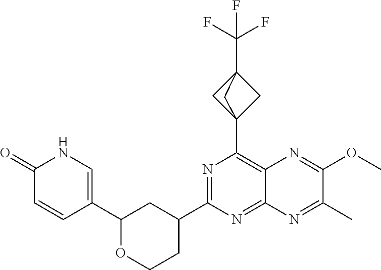 [SMILES: COc1nc2c(C34CC(C(F)(F)F)(C3)C4)nc(C3CCOC(c4ccc(=O)[nH]c4)C3)nc2nc1C] |
| 240 | 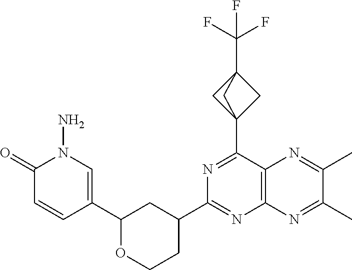 [SMILES: Cc1nc2nc(C3CCOC(c4ccc(=O)n(N)c4)C3)nc(C34CC(C(F)(F)F)(C3)C4)c2nc1C] |
| 241 | 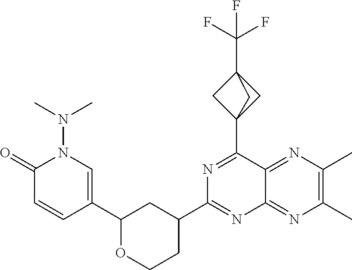 [SMILES: Cc1nc2nc(C3CCOC(c4ccc(=O)n(N(C)C)c4)C3)nc(C34CC(C(F)(F)F)(C3)C4)c2nc1C] |
| 242 | 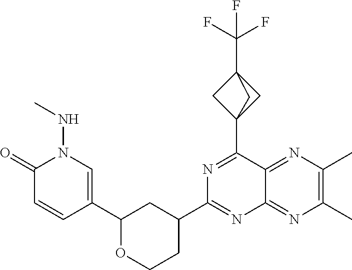 [SMILES: CNn1cc(C2CC(c3nc(C45CC(C(F)(F)F)(C4)C5)c4nc(C)c(C)nc4n3)CCO2)ccc1=O] |
| 244 | 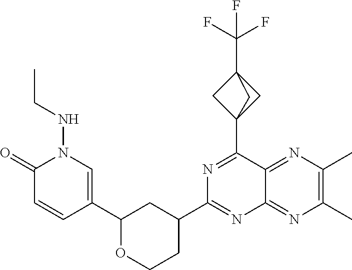 [SMILES: CCNn1cc(C2CC(c3nc(C45CC(C(F)(F)F)(C4)C5)c4nc(C)c(C)nc4n3)CCO2)ccc1=O] |
| 250 | 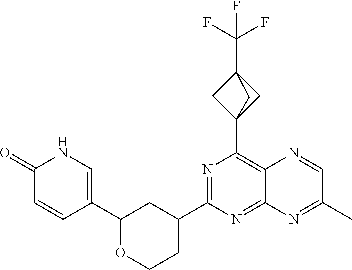 [SMILES: Cc1cnc2c(C34CC(C(F)(F)F)(C3)C4)nc(C3CCOC(c4ccc(=O)[nH]c4)C3)nc2n1] |
| 251 | 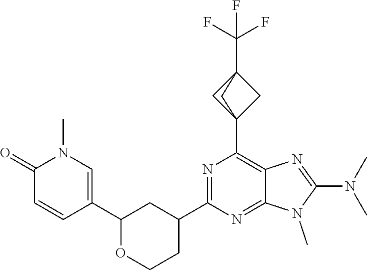 [SMILES: CN(C)c1nc2c(C34CC(C(F)(F)F)(C3)C4)nc(C3CCOC(c4ccc(=O)n(C)c4)C3)nc2n1C] |
| 257 | 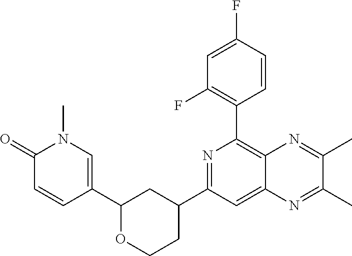 [SMILES: Cc1nc2cc(C3CCOC(c4ccc(=O)n(C)c4)C3)nc(-c3ccc(F)cc3F)c2nc1C] |
| 260 | 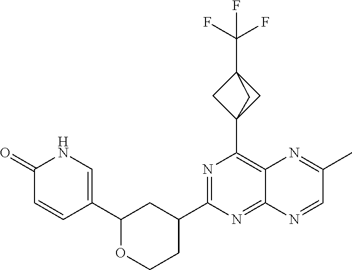 [SMILES: Cc1cnc2nc(C3CCOC(c4ccc(=O)[nH]c4)C3)nc(C34CC(C(F)(F)F)(C3)C4)c2n1] |
| 261 | 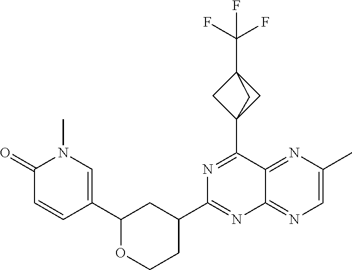 [SMILES: Cc1cnc2nc(C3CCOC(c4ccc(=O)n(C)c4)C3)nc(C34CC(C(F)(F)F)(C3)C4)c2n1] |
| 262 | 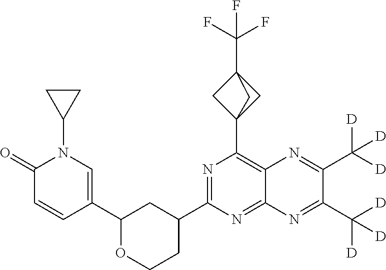 [SMILES: [2H]C([2H])([2H])c1nc2nc(C3CCOC(c4ccc(=O)n(C5CC5)c4)C3)nc(C34CC(C(F)(F)F)(C3)C4)c2nc1C([2H])([2H])[2H]] |
| 263 | 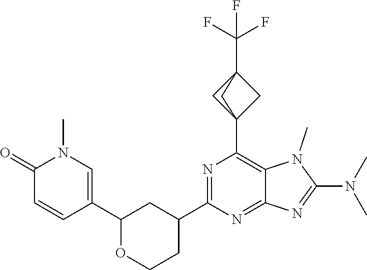 [SMILES: CN(C)c1nc2nc(C3CCOC(c4ccc(=O)n(C)c4)C3)nc(C34CC(C(F)(F)F)(C3)C4)c2n1C] |
| 264 | 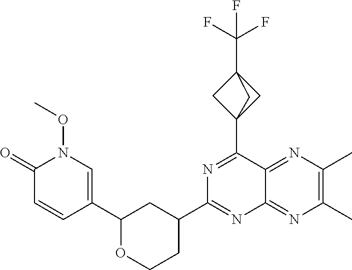 [SMILES: COn1cc(C2CC(c3nc(C45CC(C(F)(F)F)(C4)C5)c4nc(C)c(C)nc4n3)CCO2)ccc1=O] |
| 267 | 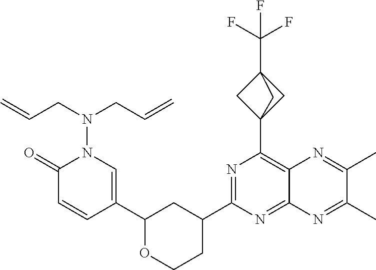 [SMILES: C=CCN(CC=C)n1cc(C2CC(c3nc(C45CC(C(F)(F)F)(C4)C5)c4nc(C)c(C)nc4n3)CCO2)ccc1=O] |
| 268 | 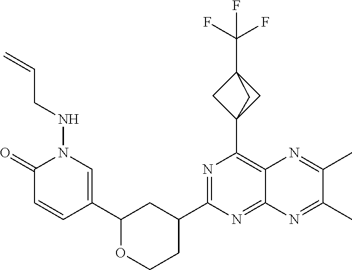 [SMILES: C=CCNn1cc(C2CC(c3nc(C45CC(C(F)(F)F)(C4)C5)c4nc(C)c(C)nc4n3)CCO2)ccc1=O] |
| 269 | 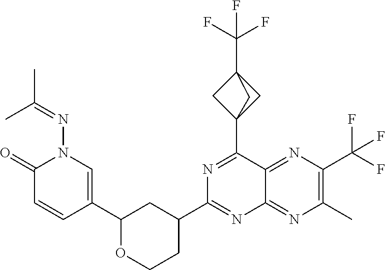 [SMILES: CC(C)=Nn1cc(C2CC(c3nc(C45CC(C(F)(F)F)(C4)C5)c4nc(C(F)(F)F)c(C)nc4n3)CCO2)ccc1=O] |
| 270 | 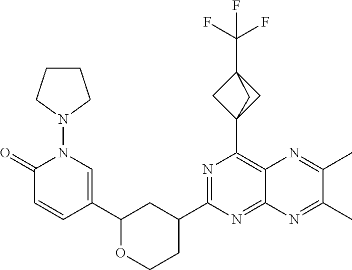 [SMILES: Cc1nc2nc(C3CCOC(c4ccc(=O)n(N5CCCC5)c4)C3)nc(C34CC(C(F)(F)F)(C3)C4)c2nc1C] |
| 271 | 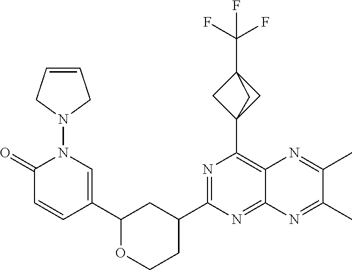 [SMILES: Cc1nc2nc(C3CCOC(c4ccc(=O)n(N5CC=CC5)c4)C3)nc(C34CC(C(F)(F)F)(C3)C4)c2nc1C] |
| 272 | 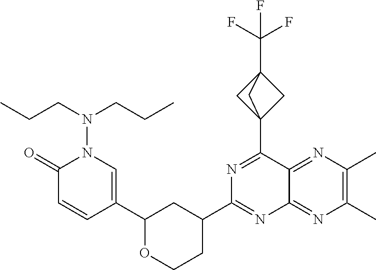 [SMILES: CCCN(CCC)n1cc(C2CC(c3nc(C45CC(C(F)(F)F)(C4)C5)c4nc(C)c(C)nc4n3)CCO2)ccc1=O] |
| 273 | 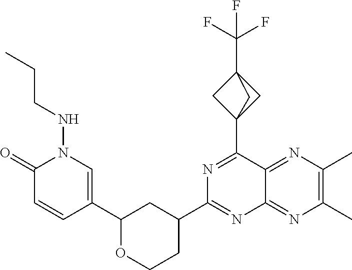 [SMILES: CCCNn1cc(C2CC(c3nc(C45CC(C(F)(F)F)(C4)C5)c4nc(C)c(C)nc4n3)CCO2)ccc1=O] |
| 274 | 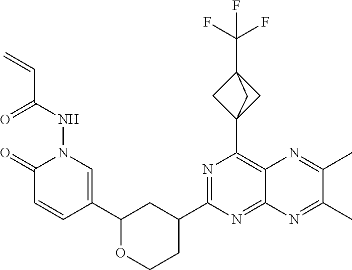 [SMILES: C=CC(=O)Nn1cc(C2CC(c3nc(C45CC(C(F)(F)F)(C4)C5)c4nc(C)c(C)nc4n3)CCO2)ccc1=O] |
| 275 | 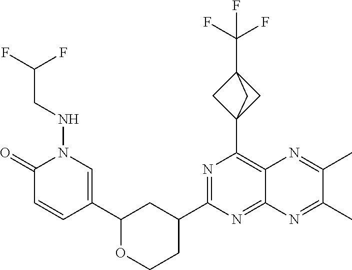 [SMILES: Cc1nc2nc(C3CCOC(c4ccc(=O)n(NCC(F)F)c4)C3)nc(C34CC(C(F)(F)F)(C3)C4)c2nc1C] |
| 276 | 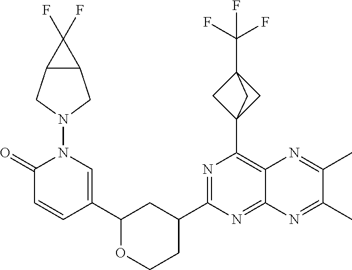 [SMILES: Cc1nc2nc(C3CCOC(c4ccc(=O)n(N5CC6C(C5)C6(F)F)c4)C3)nc(C34CC(C(F)(F)F)(C3)C4)c2nc1C] |
| 277 | 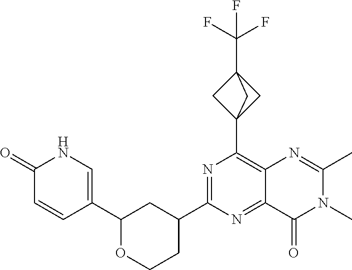 [SMILES: Cc1nc2c(C34CC(C(F)(F)F)(C3)C4)nc(C3CCOC(c4ccc(=O)[nH]c4)C3)nc2c(=O)n1C] |
| 278 | 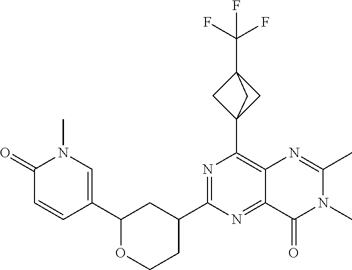 [SMILES: Cc1nc2c(C34CC(C(F)(F)F)(C3)C4)nc(C3CCOC(c4ccc(=O)n(C)c4)C3)nc2c(=O)n1C] |
| 279 | 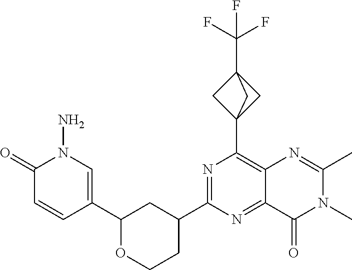 [SMILES: Cc1nc2c(C34CC(C(F)(F)F)(C3)C4)nc(C3CCOC(c4ccc(=O)n(N)c4)C3)nc2c(=O)n1C] |
| 285 | 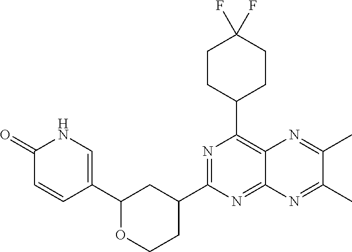 [SMILES: Cc1nc2nc(C3CCOC(c4ccc(=O)[nH]c4)C3)nc(C3CCC(F)(F)CC3)c2nc1C] |
| 286 | 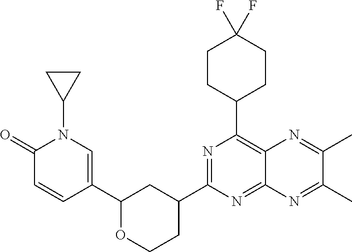 [SMILES: Cc1nc2nc(C3CCOC(c4ccc(=O)n(C5CC5)c4)C3)nc(C3CCC(F)(F)CC3)c2nc1C] |
| 287 | 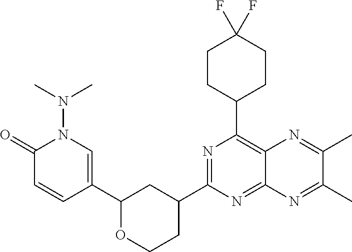 [SMILES: Cc1nc2nc(C3CCOC(c4ccc(=O)n(N(C)C)c4)C3)nc(C3CCC(F)(F)CC3)c2nc1C] |
| 288 | 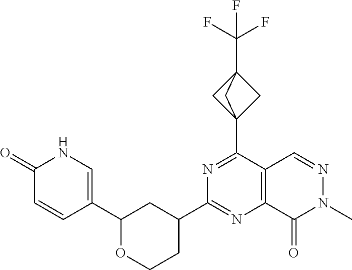 [SMILES: Cn1ncc2c(C34CC(C(F)(F)F)(C3)C4)nc(C3CCOC(c4ccc(=O)[nH]c4)C3)nc2c1=O] |
| 289 | 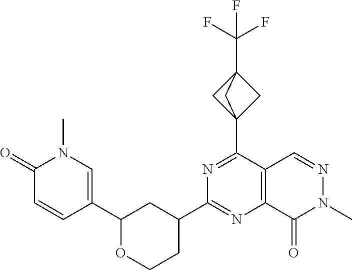 [SMILES: Cn1cc(C2CC(c3nc(C45CC(C(F)(F)F)(C4)C5)c4cnn(C)c(=O)c4n3)CCO2)ccc1=O] |
| 290 | 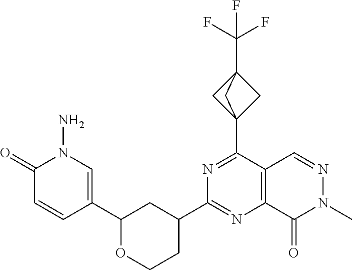 [SMILES: Cn1ncc2c(C34CC(C(F)(F)F)(C3)C4)nc(C3CCOC(c4ccc(=O)n(N)c4)C3)nc2c1=O] |
| 291 | 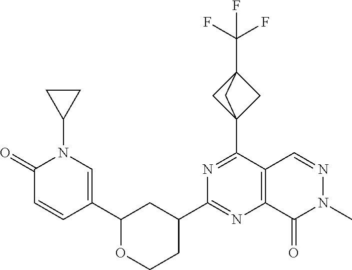 [SMILES: Cn1ncc2c(C34CC(C(F)(F)F)(C3)C4)nc(C3CCOC(c4ccc(=O)n(C5CC5)c4)C3)nc2c1=O] |
| 292 | 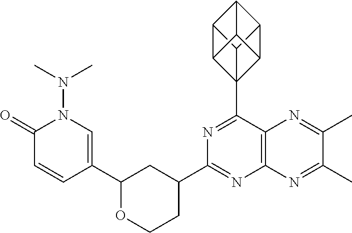 [SMILES: Cc1nc2nc(C3CCOC(c4ccc(=O)n(N(C)C)c4)C3)nc(C34C5C6C7C5C3C7C64)c2nc1C] |
| 293 | 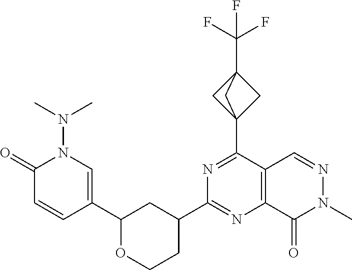 [SMILES: CN(C)n1cc(C2CC(c3nc(C45CC(C(F)(F)F)(C4)C5)c4cnn(C)c(=O)c4n3)CCO2)ccc1=O] |
| 294 | 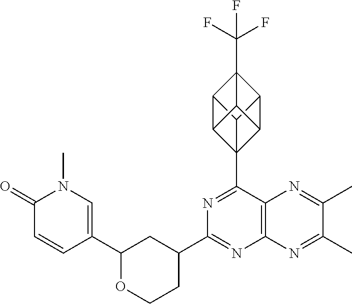 [SMILES: Cc1nc2nc(C3CCOC(c4ccc(=O)n(C)c4)C3)nc(C34C5C6C3C3C4C5C63C(F)(F)F)c2nc1C] |
| 295 | 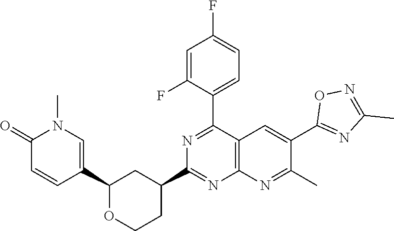 [SMILES: Cc1noc(-c2cc3c(-c4ccc(F)cc4F)nc([C@H]4CCO[C@@H](c5ccc(=O)n(C)c5)C4)nc3nc2C)n1] |
| 296 | 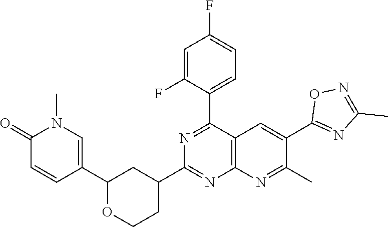 [SMILES: Cc1noc(-c2cc3c(-c4ccc(F)cc4F)nc(C4CCOC(c5ccc(=O)n(C)c5)C4)nc3nc2C)n1] |
| 297 | 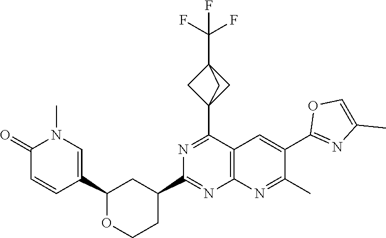 [SMILES: Cc1coc(-c2cc3c(C45CC(C(F)(F)F)(C4)C5)nc([C@H]4CCO[C@@H](c5ccc(=O)n(C)c5)C4)nc3nc2C)n1] |
| 298 | 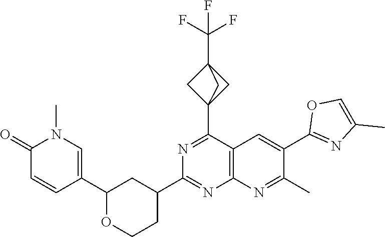 [SMILES: Cc1coc(-c2cc3c(C45CC(C(F)(F)F)(C4)C5)nc(C4CCOC(c5ccc(=O)n(C)c5)C4)nc3nc2C)n1] |
| 299 | 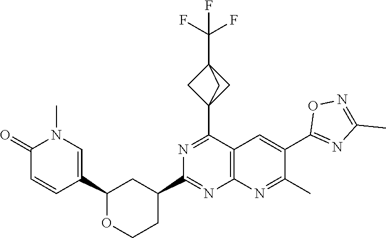 [SMILES: Cc1noc(-c2cc3c(C45CC(C(F)(F)F)(C4)C5)nc([C@H]4CCO[C@@H](c5ccc(=O)n(C)c5)C4)nc3nc2C)n1] |
| 300 | 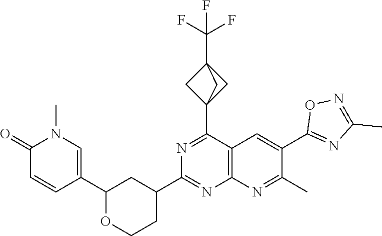 [SMILES: Cc1noc(-c2cc3c(C45CC(C(F)(F)F)(C4)C5)nc(C4CCOC(c5ccc(=O)n(C)c5)C4)nc3nc2C)n1] |
| 301 | 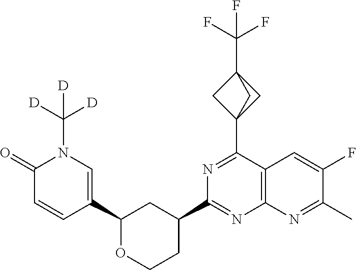 [SMILES: [2H]C([2H])([2H])n1cc([C@H]2C[C@@H](c3nc(C45CC(C(F)(F)F)(C4)C5)c4cc(F)c(C)nc4n3)CCO2)ccc1=O] |
| 302 | 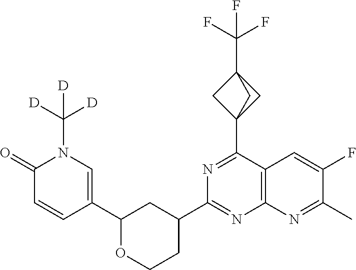 [SMILES: [2H]C([2H])([2H])n1cc(C2CC(c3nc(C45CC(C(F)(F)F)(C4)C5)c4cc(F)c(C)nc4n3)CCO2)ccc1=O] |
| 303 | 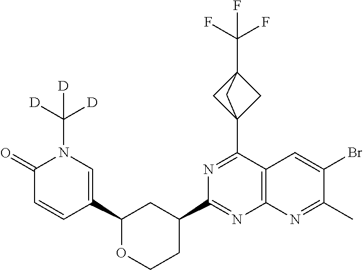 [SMILES: [2H]C([2H])([2H])n1cc([C@H]2C[C@@H](c3nc(C45CC(C(F)(F)F)(C4)C5)c4cc(Br)c(C)nc4n3)CCO2)ccc1=O] |
| 304 | 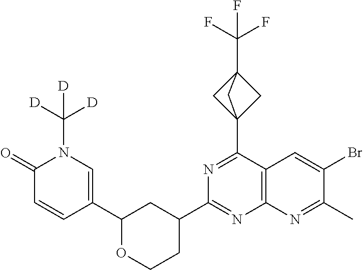 [SMILES: [2H]C([2H])([2H])n1cc(C2CC(c3nc(C45CC(C(F)(F)F)(C4)C5)c4cc(Br)c(C)nc4n3)CCO2)ccc1=O] |
| 305 | 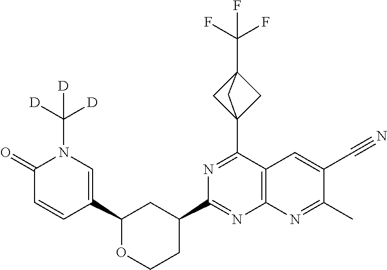 [SMILES: [2H]C([2H])([2H])n1cc([C@H]2C[C@@H](c3nc(C45CC(C(F)(F)F)(C4)C5)c4cc(C#N)c(C)nc4n3)CCO2)ccc1=O] |
| 306 | 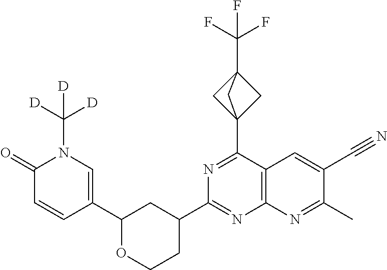 [SMILES: [2H]C([2H])([2H])n1cc(C2CC(c3nc(C45CC(C(F)(F)F)(C4)C5)c4cc(C#N)c(C)nc4n3)CCO2)ccc1=O] |
| 307 | 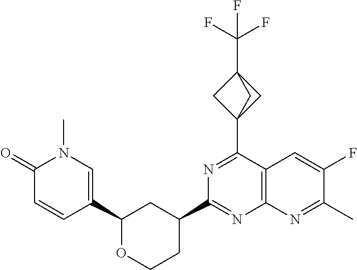 [SMILES: Cc1nc2nc([C@H]3CCO[C@@H](c4ccc(=O)n(C)c4)C3)nc(C34CC(C(F)(F)F)(C3)C4)c2cc1F] |
| 308 | 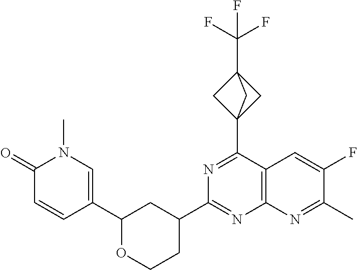 [SMILES: Cc1nc2nc(C3CCOC(c4ccc(=O)n(C)c4)C3)nc(C34CC(C(F)(F)F)(C3)C4)c2cc1F] |
| 309 | 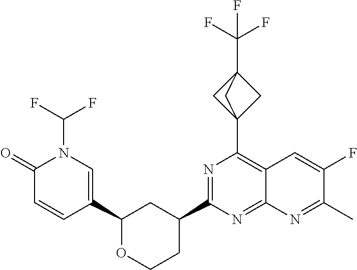 [SMILES: Cc1nc2nc([C@H]3CCO[C@@H](c4ccc(=O)n(C(F)F)c4)C3)nc(C34CC(C(F)(F)F)(C3)C4)c2cc1F] |
| 310 | 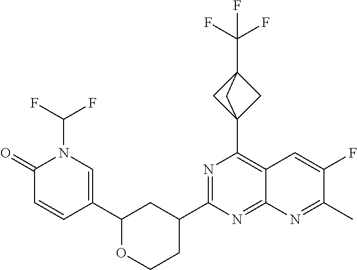 [SMILES: Cc1nc2nc(C3CCOC(c4ccc(=O)n(C(F)F)c4)C3)nc(C34CC(C(F)(F)F)(C3)C4)c2cc1F] |
| 311 | 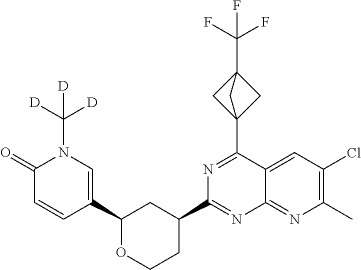 [SMILES: [2H]C([2H])([2H])n1cc([C@H]2C[C@@H](c3nc(C45CC(C(F)(F)F)(C4)C5)c4cc(Cl)c(C)nc4n3)CCO2)ccc1=O] |
| 312 | 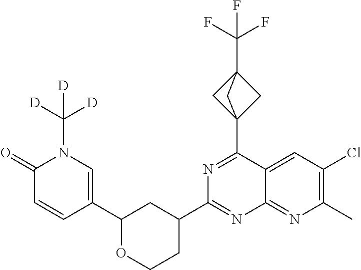 [SMILES: [2H]C([2H])([2H])n1cc(C2CC(c3nc(C45CC(C(F)(F)F)(C4)C5)c4cc(Cl)c(C)nc4n3)CCO2)ccc1=O] |
| 313 | 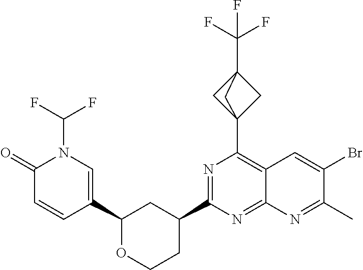 [SMILES: Cc1nc2nc([C@H]3CCO[C@@H](c4ccc(=O)n(C(F)F)c4)C3)nc(C34CC(C(F)(F)F)(C3)C4)c2cc1Br] |
| 314 | 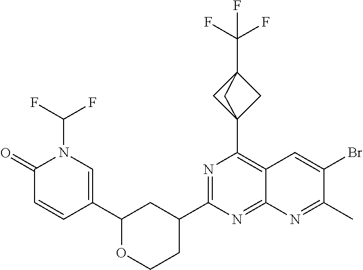 [SMILES: Cc1nc2nc(C3CCOC(c4ccc(=O)n(C(F)F)c4)C3)nc(C34CC(C(F)(F)F)(C3)C4)c2cc1Br] |
| 315 | 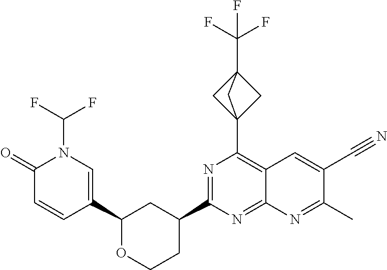 [SMILES: Cc1nc2nc([C@H]3CCO[C@@H](c4ccc(=O)n(C(F)F)c4)C3)nc(C34CC(C(F)(F)F)(C3)C4)c2cc1C#N] |
| 316 | 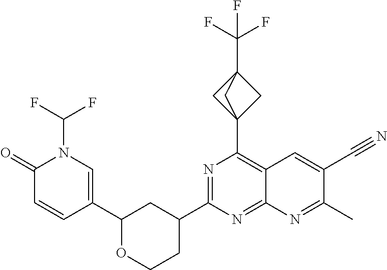 [SMILES: Cc1nc2nc(C3CCOC(c4ccc(=O)n(C(F)F)c4)C3)nc(C34CC(C(F)(F)F)(C3)C4)c2cc1C#N] |
| 317 | 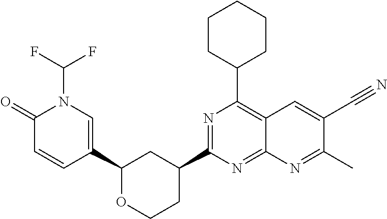 [SMILES: Cc1nc2nc([C@H]3CCO[C@@H](c4ccc(=O)n(C(F)F)c4)C3)nc(C3CCCCC3)c2cc1C#N] |
| 318 | 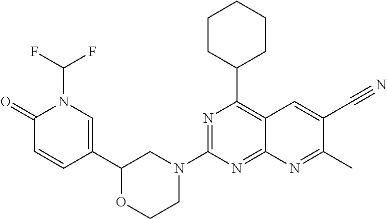 [SMILES: Cc1nc2nc(N3CCOC(c4ccc(=O)n(C(F)F)c4)C3)nc(C3CCCCC3)c2cc1C#N] |
| 319 | 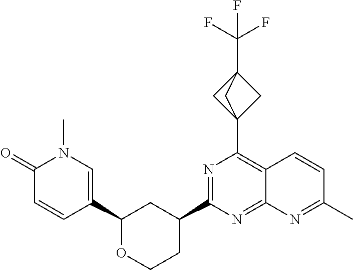 [SMILES: Cc1ccc2c(C34CC(C(F)(F)F)(C3)C4)nc([C@H]3CCO[C@@H](c4ccc(=O)n(C)c4)C3)nc2n1] |
| 320 | 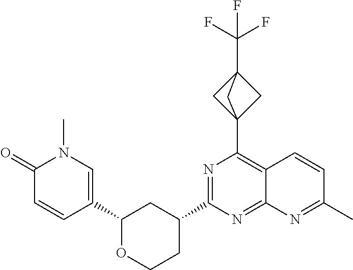 [SMILES: Cc1ccc2c(C34CC(C(F)(F)F)(C3)C4)nc([C@@H]3CCO[C@H](c4ccc(=O)n(C)c4)C3)nc2n1] |
| 321 | 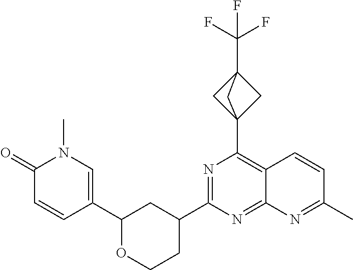 [SMILES: Cc1ccc2c(C34CC(C(F)(F)F)(C3)C4)nc(C3CCOC(c4ccc(=O)n(C)c4)C3)nc2n1] |
| 322 | 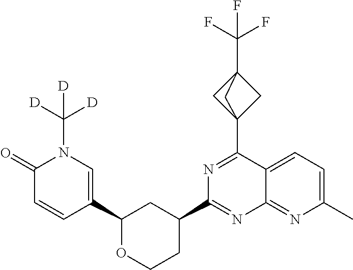 [SMILES: [2H]C([2H])([2H])n1cc([C@H]2C[C@@H](c3nc(C45CC(C(F)(F)F)(C4)C5)c4ccc(C)nc4n3)CCO2)ccc1=O] |
| 323 | 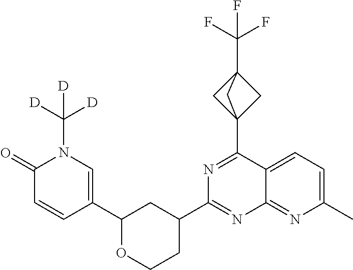 [SMILES: [2H]C([2H])([2H])n1cc(C2CC(c3nc(C45CC(C(F)(F)F)(C4)C5)c4ccc(C)nc4n3)CCO2)ccc1=O] |
| 324 | 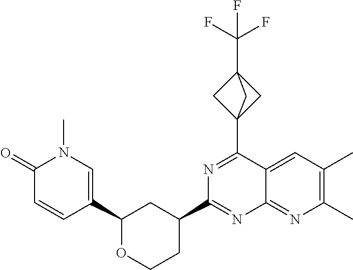 [SMILES: Cc1cc2c(C34CC(C(F)(F)F)(C3)C4)nc([C@H]3CCO[C@@H](c4ccc(=O)n(C)c4)C3)nc2nc1C] |
| 325 | 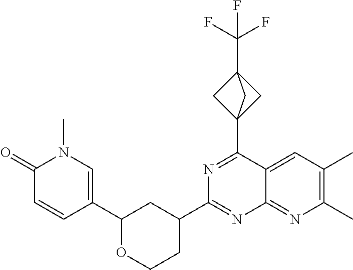 [SMILES: Cc1cc2c(C34CC(C(F)(F)F)(C3)C4)nc(C3CCOC(c4ccc(=O)n(C)c4)C3)nc2nc1C] |
| 326 | 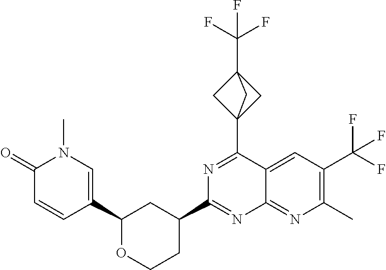 [SMILES: Cc1nc2nc([C@H]3CCO[C@@H](c4ccc(=O)n(C)c4)C3)nc(C34CC(C(F)(F)F)(C3)C4)c2cc1C(F)(F)F] |
| 327 | 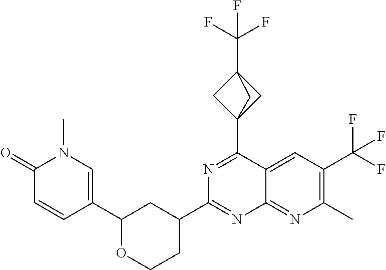 [SMILES: Cc1nc2nc(C3CCOC(c4ccc(=O)n(C)c4)C3)nc(C34CC(C(F)(F)F)(C3)C4)c2cc1C(F)(F)F] |
| 328 | 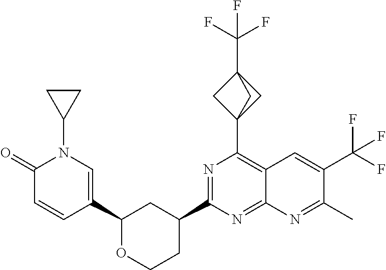 [SMILES: Cc1nc2nc([C@H]3CCO[C@@H](c4ccc(=O)n(C5CC5)c4)C3)nc(C34CC(C(F)(F)F)(C3)C4)c2cc1C(F)(F)F] |
| 329 | 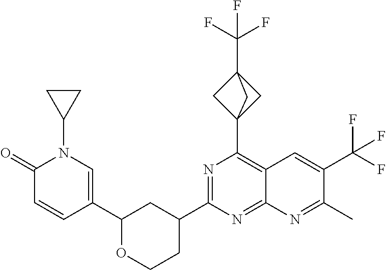 [SMILES: Cc1nc2nc(C3CCOC(c4ccc(=O)n(C5CC5)c4)C3)nc(C34CC(C(F)(F)F)(C3)C4)c2cc1C(F)(F)F] |
| 330 | 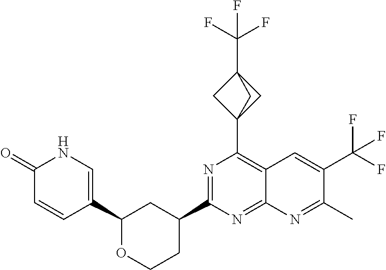 [SMILES: Cc1nc2nc([C@H]3CCO[C@@H](c4ccc(=O)[nH]c4)C3)nc(C34CC(C(F)(F)F)(C3)C4)c2cc1C(F)(F)F] |
| 331 | 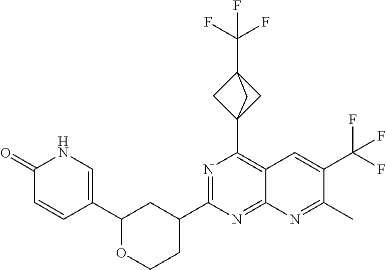 [SMILES: Cc1nc2nc(C3CCOC(c4ccc(=O)[nH]c4)C3)nc(C34CC(C(F)(F)F)(C3)C4)c2cc1C(F)(F)F] |
| 332 | 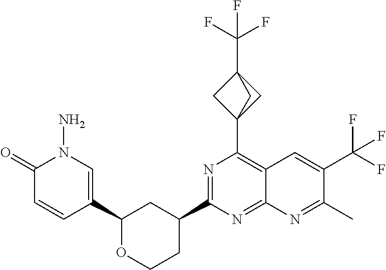 [SMILES: Cc1nc2nc([C@H]3CCO[C@@H](c4ccc(=O)n(N)c4)C3)nc(C34CC(C(F)(F)F)(C3)C4)c2cc1C(F)(F)F] |
| 333 | 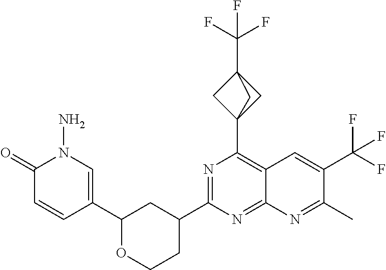 [SMILES: Cc1nc2nc(C3CCOC(c4ccc(=O)n(N)c4)C3)nc(C34CC(C(F)(F)F)(C3)C4)c2cc1C(F)(F)F] |
| 334 | 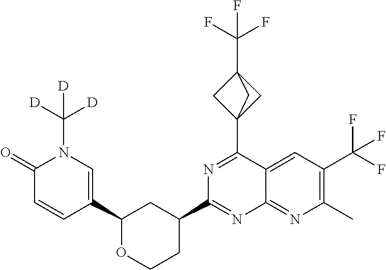 [SMILES: [2H]C([2H])([2H])n1cc([C@H]2C[C@@H](c3nc(C45CC(C(F)(F)F)(C4)C5)c4cc(C(F)(F)F)c(C)nc4n3)CCO2)ccc1=O] |
| 335 | 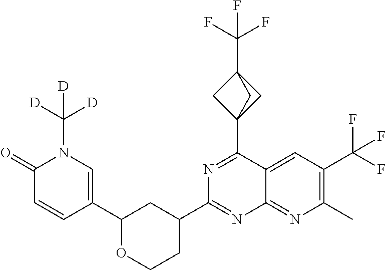 [SMILES: [2H]C([2H])([2H])n1cc(C2CC(c3nc(C45CC(C(F)(F)F)(C4)C5)c4cc(C(F)(F)F)c(C)nc4n3)CCO2)ccc1=O] |
| 336 | 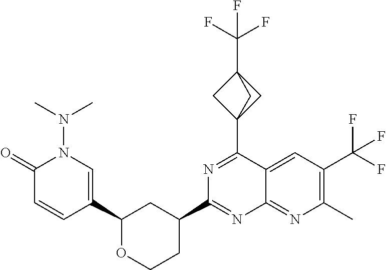 [SMILES: Cc1nc2nc([C@H]3CCO[C@@H](c4ccc(=O)n(N(C)C)c4)C3)nc(C34CC(C(F)(F)F)(C3)C4)c2cc1C(F)(F)F] |
| 337 | 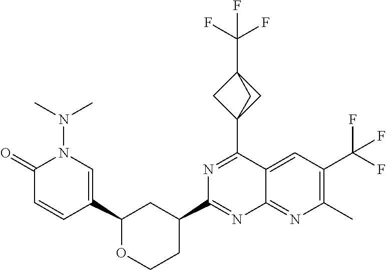 [SMILES: Cc1nc2nc([C@H]3CCO[C@@H](c4ccc(=O)n(N(C)C)c4)C3)nc(C34CC(C(F)(F)F)(C3)C4)c2cc1C(F)(F)F] |
| 338 | 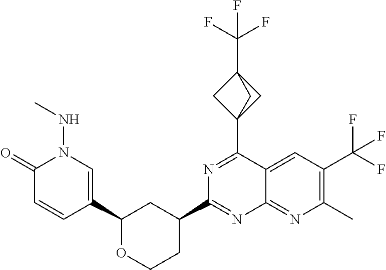 [SMILES: CNn1cc([C@H]2C[C@@H](c3nc(C45CC(C(F)(F)F)(C4)C5)c4cc(C(F)(F)F)c(C)nc4n3)CCO2)ccc1=O] |
| 339 | 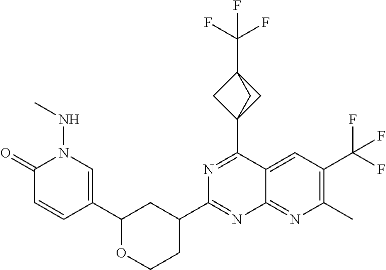 [SMILES: CNn1cc(C2CC(c3nc(C45CC(C(F)(F)F)(C4)C5)c4cc(C(F)(F)F)c(C)nc4n3)CCO2)ccc1=O] |
| 342 | 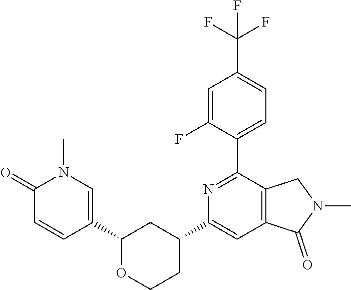 [SMILES: CN1Cc2c(cc([C@@H]3CCO[C@H](c4ccc(=O)n(C)c4)C3)nc2-c2ccc(C(F)(F)F)cc2F)C1=O] |
| 343 | 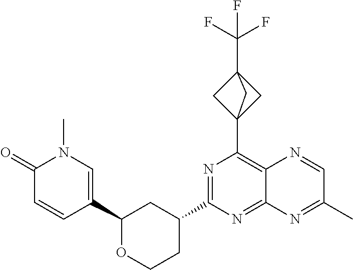 [SMILES: Cc1cnc2c(C34CC(C(F)(F)F)(C3)C4)nc([C@@H]3CCO[C@@H](c4ccc(=O)n(C)c4)C3)nc2n1] |
| 344 | 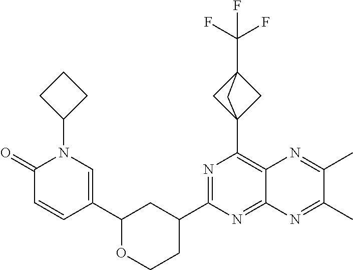 [SMILES: Cc1nc2nc(C3CCOC(c4ccc(=O)n(C5CCC5)c4)C3)nc(C34CC(C(F)(F)F)(C3)C4)c2nc1C] |
| 345 | 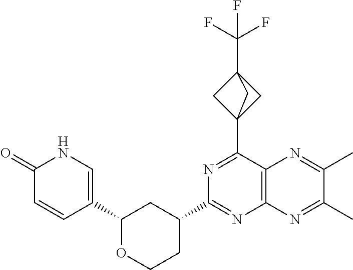 [SMILES: Cc1nc2nc([C@@H]3CCO[C@H](c4ccc(=O)[nH]c4)C3)nc(C34CC(C(F)(F)F)(C3)C4)c2nc1C] |
| 346 | 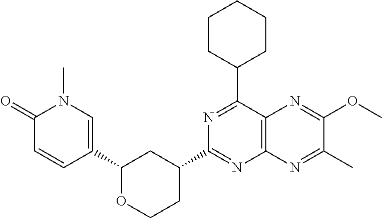 [SMILES: COc1nc2c(C3CCCCC3)nc([C@@H]3CCO[C@H](c4ccc(=O)n(C)c4)C3)nc2nc1C] |
| 347 | 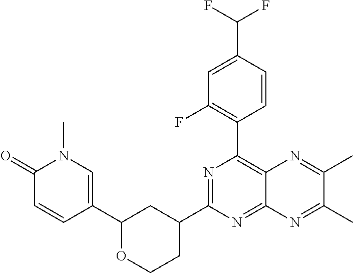 [SMILES: Cc1nc2nc(C3CCOC(c4ccc(=O)n(C)c4)C3)nc(-c3ccc(C(F)F)cc3F)c2nc1C] |
| 348 | 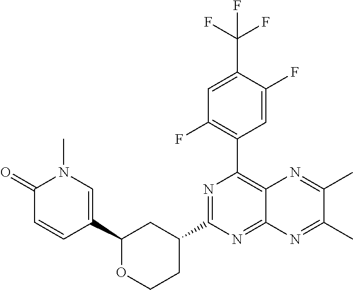 [SMILES: Cc1nc2nc([C@@H]3CCO[C@@H](c4ccc(=O)n(C)c4)C3)nc(-c3cc(F)c(C(F)(F)F)cc3F)c2nc1C] |
| 349 | 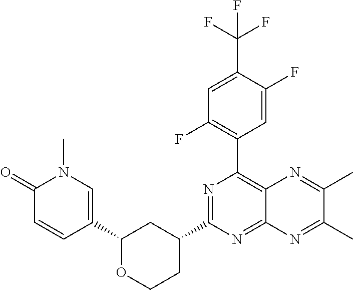 [SMILES: Cc1nc2nc([C@@H]3CCO[C@H](c4ccc(=O)n(C)c4)C3)nc(-c3cc(F)c(C(F)(F)F)cc3F)c2nc1C] |
| 350 | 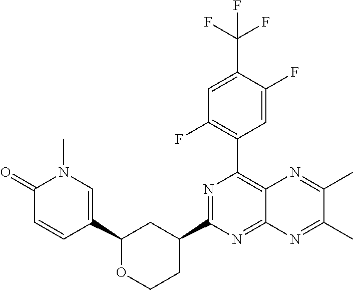 [SMILES: Cc1nc2nc([C@H]3CCO[C@@H](c4ccc(=O)n(C)c4)C3)nc(-c3cc(F)c(C(F)(F)F)cc3F)c2nc1C] |
| 351 | 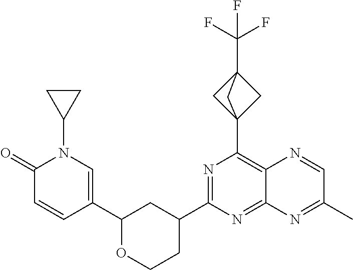 [SMILES: Cc1cnc2c(C34CC(C(F)(F)F)(C3)C4)nc(C3CCOC(c4ccc(=O)n(C5CC5)c4)C3)nc2n1] |
| 352 | 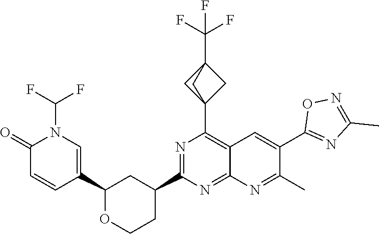 [SMILES: Cc1noc(-c2cc3c(C45CC(C(F)(F)F)(C4)C5)nc([C@H]4CCO[C@@H](c5ccc(=O)n(C(F)F)c5)C4)nc3nc2C)n1] |
| 353 | 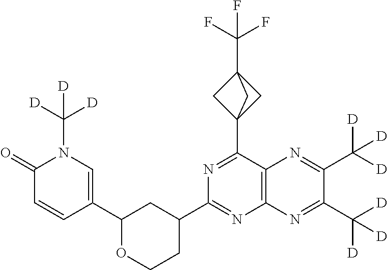 [SMILES: [2H]C([2H])([2H])c1nc2nc(C3CCOC(c4ccc(=O)n(C([2H])([2H])[2H])c4)C3)nc(C34CC(C(F)(F)F)(C3)C4)c2nc1C([2H])([2H])[2H]] |
| 355 | 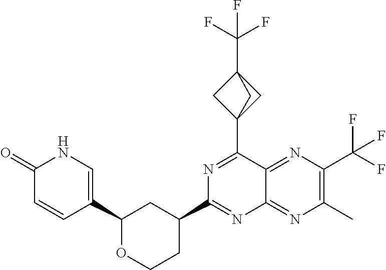 [SMILES: Cc1nc2nc([C@H]3CCO[C@@H](c4ccc(=O)[nH]c4)C3)nc(C34CC(C(F)(F)F)(C3)C4)c2nc1C(F)(F)F] |

98. A pharmaceutical composition comprising the compound of claim 2, or a pharmaceutically acceptable salt thereof, and a pharmaceutically acceptable excipient.

99. A pharmaceutical composition comprising the compound of claim 3, or a pharmaceutically acceptable salt thereof, and a pharmaceutically acceptable excipient.
